(19) United States
(12) Patent Application Publication
(10) Pub. No.: US 2023/0101362 A1
(43) Pub. Date: Mar. 30, 2023

(54) RNA STABILIZATION

(71) Applicant: Team Medical LLC, Boulder, CO (US)

(72) Inventors: Kyle P. HEIM, Boulder, CO (US); Warren P. HEIM, Boulder, CO (US)

(21) Appl. No.: 17/932,978

(22) Filed: Sep. 16, 2022

Related U.S. Application Data
(60) Provisional application No. 63/244,767, filed on Sep. 16, 2021.
(60) Provisional application No. 63/264,325, filed on Nov. 19, 2021.
(60) Provisional application No. 63/365,011, filed on May 19, 2022.

(51) Int. Cl. C12N 15/10; C12N 15/113

(52) U.S. Cl. CPC C12N 15/1024; C12N 15/113; C12P 19/34

(57) ABSTRACT

Formulations of substances comprising at least one RNA stabilizing substance and at least one substance comprising RNA or based on RNA and methods of using the formulations to improve the storage and use stability of substances comprising RNA or based on RNA.

### CROSS-REFERENCE TO RELATED APPLICATIONS

This application claims priority to the U.S. Provisional Application No. 63/244,767, filed on Sep. 16, 2021, entitled “RNA Stabilization”, U.S. Provisional Application No. 63/264,325, filed on Nov. 19, 2021, entitled “RNA STABILIZATION”, and U.S. Provisional Application No. 63/365,011, filed on May 19, 2022, entitled “RNA Stabilization”. The contents of all of the above-noted applications are incorporated herein by reference as if set forth in full and priority to these applications is claimed to the full extent allowable under U.S. law and regulations.

### FIELD OF THE INVENTION

The present invention relates to formulations and methods of using the formulations to improve the stability of various types of extracellular RNA and substances based on various types of extracellular RNA for storage and use in non-clinical applications and clinical applications including for therapeutic uses to diagnose the health or improve the health of living organisms including plants and animals including treating humans including diagnosis of diseases and treatment of diseases or other adverse health effects in animals including in humans.

### BACKGROUND OF THE INVENTION

Ribonucleic acid (RNA) is responsible for the transcription of the genetic information stored in deoxyribonucleic acid (DNA) in a form that can be used in cells to synthesize proteins. The use of therapeutic RNA to beneficially produce proteins, regulate gene expression, or induce immune responses to specific antigens and biomarkers has become an emerging part of the field of nucleic acid therapy. The potential applications and recognized advantages of RNA based nucleic acid therapies continue to increase. For example, the recent COVID-19 infections in humans have led to vaccines developed using messenger RNA (mRNA). RNA therapies, such as vaccines using mRNA have advantages compared to other therapies, such as traditional vaccines that use inactivated, attenuated, or genetically modified microorganisms, purified antigens, or viral vectors. These other therapies can lead to adverse reactions, side effects, allergic reactions, or can develop mutations, either during manufacturing or administration, that can alter the efficacy or lead to safety concerns. RNA encodes the genetic information for the target therapy to be produced endogenously within the host cell without the need to synthesize and purify individual antigens, thereby creating greater flexibility to specifically tailor different therapies for a variety of diseases and simplifying the manufacturing process by allowing the target cells to facilitate the production of the necessary proteins and reduce or eliminate the complications of traditional vaccines.

Using RNA for nucleic acid therapies has distinct advantages compared to DNA based therapies due to the relatively short half-life of RNA and the transient message encoded within the RNA that does not require entry into the nucleus for proper expression necessary to carry out the desired function. Furthermore, DNA can potentially integrate into the host cell genome and alter genomic DNA or also become inherited by progeny. However, the investigation of uses of RNA and the production of products using RNA is complicated by the limited stability of RNA. RNA is not as stable as DNA due to RNA's single stranded nature in many biological systems and the substitution of ribose within the sugar phosphate backbone (instead of deoxyribose in DNA), leading to the presence of a 2′-hydroxyl group within the structure of the RNA backbone. The single stranded nature of RNA and the presence of the 2′-hydroxyl makes RNA susceptible to hydrolysis, in which the RNA molecule is cleaved by breaking the phosphodiester bond in the sugar-phosphate backbone, leading to degradation of the RNA molecule. Storing RNA, including storing mRNA-based vaccines, requires conditions that slow or prevent RNA from degrading, as described below, and interfering with the desired effects that RNA induces in targeted living cells.

Storage at extreme low temperatures below the freezing point of water, such as at or below about −20° C. or even at or below about −80° C., is known to be useful or even required for durable storage of RNA, including, but not limited to, for example, vaccines based on mRNA.

Storing at extreme low temperatures is more complicated than storing at more easily achieved temperatures such as temperatures approximately at the freezing point of water or refrigerated temperatures or even room temperatures or other ambient temperatures. Among the complications associated with temperatures below ambient temperatures are that refrigeration means are needed. Such refrigeration means includes cooling using thermodynamic cycles such as mechanical refrigeration (including freezers and refrigerators), frozen carbon dioxide (dry ice), or frozen water (ice).

Storing at extreme low temperatures such as at or below about −70° C. or even at or below about −80° C. or at or below about −20° C. or at or below about 4° C. complicates and adds expense to storing substances containing or based on RNA, including the storage, transportation, and therapeutic access for administration of vaccines. The complexity of using RNA is reduced the closer to room temperatures or other ambient temperatures that RNA substances can be stable.

To reduce the complexity of storing RNA substances, including mRNA and substances containing or based on mRNA such as mRNA vaccines or other therapeutic products, materials and methods for improving the stability of RNA substances so that storage can be done at temperatures greater than extremely cold temperatures are needed.

Lyophilization or freeze-drying RNA substances is used to improve the storage stability of RNA and reduce the need for storing RNA at cold temperatures or even extreme cold temperatures. However, freeze-drying and lyophilization requires specialized equipment and adds significant time, expense, and complexity to the production and storage of RNA, including but not limited to mRNA and substances containing or based on mRNA such as mRNA vaccines or other therapeutic products.

Encapsulating or complexing RNA substances with nanoparticles or lipid nanoparticles is used to improve the delivery of an RNA substance to a cell or tissue. Nanoparticles may incorporate polyethylene glycol (PEG) modified lipids, PEG conjugated lipids, or similar modifications to improve nanoparticle stability. These modifications improve the stability of the nanoparticle by decreasing aggregation and agglomeration, as well as reducing protein binding and opsonization. However, improving nanoparticle stability relates to maintaining consistent nanoparticle size and distribution as well as improving circulation half-life and reducing systemic clearance of nanoparticles following administration of the encapsulated or complexed RNA and does not necessarily improve RNA stability by preventing RNA degradation during storage or shipping or reducing the need for storing or shipping RNA at cold temperatures or even extreme cold temperatures.

Aprotic substances used during nucleic acid or RNA synthesis are not necessarily used to improve RNA stability by preventing RNA degradation during storage or shipping and are not necessarily used to reduce the need for storing or shipping RNA at cold temperatures or even extreme cold temperatures following synthesis. Instead of being used to improve RNA stability during storage or shipping by reducing RNA degradation, aprotic substances, including DMSO, are often used during PCR or DNA or RNA synthesis to denature or decrease DNA or RNA duplex stability and disrupt or alter secondary structure of RNA or DNA to help facilitate synthesis of GC rich regions or GC rich templates and denature double stranded nucleic acid or reduce sequence variability associated with the efficiency of nucleic acid synthesis. These aprotic substances, such as DMSO, help reduce the melting temperature (Tm) of nucleic acid secondary structure or base paring or reduce the Tm needed to denature double stranded nucleic acid, allowing enzymes involved in transcription or nucleic acid synthesis to efficiently read the template nucleic acid strand. The use of aprotic substances in the context of reducing or altering secondary structure or duplex stability (including denaturing double stranded nucleic acid) during nucleic acid synthesis relates to improving the efficiency of synthesis by disrupting base-pairing interactions, reducing or altering DNA or RNA secondary structure or duplex stability and are not used for the purpose of preventing or reducing RNA degradation during storage or shipping. Furthermore, following the use of aprotic substances to reduce or alter the secondary structure and improve efficiency of nucleic acid synthesis, the synthesized nucleic acids are purified following the synthesis and the aprotic substances used during synthesis are removed.

### SUMMARY OF THE INVENTION

Accordingly, a primary objective of the present invention is to provide substances for storage environments for RNA substances and methods using substances in storage environments for RNA substances that reduce degradation of RNA substances so that RNA substances have improved stability when stored at temperatures above about −80° C. As used herein, the terms RNA substance or RNA substances means a substance or substances comprised of at least one of extracellular RNA or purified extracellular RNA and may include but is not limited to, mRNA and vaccines, therapeutics, or medicaments, based on mRNA. As used herein, the term storage environment means the substances in which one or more RNA substance is present other than when being synthesized or transcribed or deployed for immediate use.

Another primary objective of the present invention is to provide substances for storage environments for RNA substances and methods using substances in storage environments for RNA substances that reduces degradation of RNA substances so that RNA substances have improved stability when stored at temperatures at or above about −20° C. Another primary objective of the present invention is to provide substances for storage environments and methods using substances in storage environments for RNA substances that reduces degradation of RNA substances so that RNA substances have improved stability when stored at temperatures at or above about 4° C. Another primary objective of the present invention is to provide substances for storage environments and methods using substances in storage environments for RNA substances that reduces degradation of RNA substances so that RNA substances have improved stability when stored at temperatures at or above about 20° C.

Another primary objective of the present invention is to provide storage environments for RNA substances that reduce the needs for storing, transporting, distributing, or storing at a point of use such as a point for therapeutic administration (collectively “storage” or “storage and use” hereinafter) using thermodynamic cycle cooling such as vapor compression mechanical cooling or absorption cooling. Another primary objective of the present invention is to provide storage environments for RNA substances that reduce the needs for storage and use of RNA substances using dry ice. Another primary objective of the present invention is to provide storage environments for RNA substances that reduce the needs for storage and use of RNA substances using ice. Another primary objective of the present invention is to provide storage environments for RNA substances that reduce the needs for storage and use of RNA substances at temperatures of about −80° C. Another primary objective of the present invention is to provide storage environments for RNA substances that reduce the needs for storage and use of RNA substances at temperatures of about −20° C. Another primary objective of the present invention is to provide storage environments for RNA substances that reduce the needs for storage and use of RNA substances at temperatures of about 4° C.

The storage environment for RNA substances includes an environment that contains at least one or more RNA stabilizing substances that is combined with at least one RNA substance such as by mixing, pipetting, blending, submerging, vortexing, shaking, lyophilizing, vaporizing, or sublimating such that the RNA stabilizing substance is at least intimately associated with or partially contacting or at least partially encapsulating the RNA substance. The storage environment may include at least one RNA stabilizing substance and at least one RNA substance that are stored separately with the intention of combining or mixing the RNA substance with the RNA stabilizing substance either prior to or during use of the RNA substance. The storage environment includes the immediate environment of the RNA substance such as occurs when the RNA substance is mixed or is otherwise in close association or at least partially or substantially contacting one or more RNA stabilizing substances. Dimethyl sulfoxide (DMSO) is an example RNA stabilizing substance that may be used. As a non-limiting example, the storage environment for RNA substances may be at least some DMSO intimately contacting at least part of one or more RNA substances at the molecular level such as may result from submerging, blending, or mixing one or more RNA substances with the aprotic substance DMSO.

The inventors have discovered that aprotic substances, described in detail elsewhere herein, may be RNA stabilizing substances. In a non-limiting example, an RNA stabilizing substance may be an aprotic substance such as, DMSO.

The inventors have discovered that polar aprotic substances, described in detail elsewhere herein, may be RNA stabilizing substances. In a non-limiting example, an RNA stabilizing substance may be a polar aprotic substance such as, DMSO.

The inventors have discovered that mixtures comprising one or more RNA substance and at least one RNA stabilizing substance improves RNA stability. The inventors have also discovered that RNA stability may be improved with mixtures comprising one or more RNA substance and two or more RNA stabilizing substances. The inventors have also discovered that RNA stability may be improved with mixtures comprising one or more RNA substance and three or more RNA stabilizing substances. The inventors have also discovered that RNA stability may be improved with mixtures comprising one or more RNA substance and four or more RNA stabilizing substances. The inventors have also discovered that RNA stability may be improved with mixtures comprising one or more RNA substance and five or more RNA stabilizing substances.

As used herein, the terms stabilize RNA or stabilizing RNA means reducing degradation of RNA substances.

As used herein, an RNA stabilizing substance means a substance that stabilizes RNA of at least one or more RNA substance. RNA stabilizing substances provide a storage environment for the RNA substance that makes the RNA substance at least as stable at a higher temperature than the stability the RNA substance would have at a lower temperature.

As used herein, cells means in vivo, in vitro, in situ, or ex vivo cells, including but not limited to eukaryotic cells, prokaryotic cells, plant cells, fungal cells, insect cells, bacterial cells, mycoplasma, protozoa, plasmodium, or mammalian cells, including but not limited to the cells of vertebrate animals and the cells of humans.

As used herein, nucleic acid means DNA, RNA, polymeric, single-stranded, double-stranded, or more highly aggregated hybridization motifs, and may include chemical modifications, derivatives, or analogs thereof. Modifications may include but are not limited to modifications comprising backbone modifications, sugar modifications, or base modifications. Modifications may also include, but are not limited to, those providing chemical groups that incorporate additional charge, polarizability, hydrogen bonding, electrostatic interaction, points of attachment or functionality to the nucleic acid bases or to the nucleic acid as a whole. Such modifications may include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine or isoguanidine or the like. Nucleic acids may also include non-natural bases, such as, for example, nitroindole. Modifications may also include 3′ modifications or 5′ modifications such as capping with a fluorophore (e.g., quantum dot) or another moiety. Nucleotides, may be referred to by their commonly accepted single-letter codes.

As used herein, the term RNA means ribonucleic acid, and may include chemical modifications, derivatives, or analogs thereof, with the exception of a chemical modification rendering the RNA into DNA. RNA may include RNA analogs, including but not limited to synthetic RNA analogs or nucleotide analogs. RNA may also include non-natural synthetic ribonucleotides. The RNA may be polymeric, single-stranded, or double stranded, or a more highly aggregated form. The RNA may be provided by any means known in the art, including but not limited to, in vitro transcription, purification from an organism, chemical synthesis, or a combination thereof. The RNA may be, but is not limited to, mRNA, rRNA, tRNA, microRNA, small interfering RNA (siRNA), self-amplifying RNA, small activating RNA, tmRNA, dsRNA, shRNA, snRNA, antisense RNA (asRNA), eRNA, RNA enzymes, CRISPR RNA, or total RNA. The RNA may be purified RNA (e.g., purified mRNA, purified rRNA, purified tRNA, purified microRNA, purified siRNA, purified self-amplifying RNA, purified small activating RNA, purified tmRNA, purified dsRNA, purified shRNA, purified snRNA, purified asRNA, purified eRNA, purified RNA enzymes, purified CRISPR RNA, or purified total RNA). Furthermore, RNA modifications may include but are not limited to modifications comprising backbone modifications, sugar modifications, or base modifications. RNA modifications may also include, but are not limited to, 5′ modifications or 3′ modifications, and may also include, but are not limited to, 5′-cap, 5′-cap structures, 5′-cap modifications, or 5′-cap analogs. RNA modifications may also include, but are not limited to, lipid modifications or PEG modifications, wherein a lipid or polyethylene glycol may be attached or covalently linked to an RNA molecule.

As used herein, the terms RNA substance or RNA substances means a substance or substances comprised of at least one of extracellular RNA or purified extracellular RNA. RNA substance or RNA substances may include, but are not limited to, substances comprising one or more polymeric forms of RNA, including but not limited to single stranded or multiple stranded (including double stranded) forms that may include, but are not limited to, coding or non-coding forms of RNA. RNA substance or RNA substances may also include, but are not limited to, mRNA and vaccines, therapeutics, diagnostics, or medicaments based on RNA, mRNA, or sections of RNA or other forms of ribonucleic acid that may be used for, including but not limited to, therapeutic, diagnostic, analysis, in vitro, in vivo, ex vivo, in situ, delivery, manufacturing, storage or other purposes. RNA substance or RNA substances may include, but are not limited to, mRNA, self-amplifying RNA, small activating RNA, rRNA, tRNA, microRNA, siRNA, tmRNA, dsRNA, shRNA, snRNA, asRNA, eRNA, RNA enzymes, CRISPR RNA, or total RNA. RNA substance or RNA substances may include, but are not limited to, purified RNA, including but not limited to, purified mRNA, purified rRNA, purified tRNA, purified microRNA, purified siRNA, purified self-amplifying RNA, purified small activating RNA, purified tmRNA, purified dsRNA, purified shRNA, purified snRNA, purified asRNA, purified eRNA, purified RNA enzymes, purified CRISPR RNA, or purified total RNA.

RNA modifications of the present invention may include, but are not limited to, those described in US Patent Application Pub. No. US 2020/0383922, incorporated herein by reference, as RNA modifications, chemical modifications, backbone modifications, sugar modifications, base modifications, nucleotide analogues/modifications, modified nucleosides, nucleoside modifications, lipid modifications, or 5′-CAP structures.

RNA modifications of the present invention may include, but are not limited to, those described in U.S. Pat. No. 10,702,600, incorporated herein by reference, as chemical modifications, modifications of polynucleotides, modified RNA polynucleotides, nucleoside or nucleotide modifications, modified nucleobases, or naturally occurring or non-naturally occurring modifications.

RNA modifications of the present invention may include, but are not limited to, those described in WO Patent Application Pub. No. WO 2021/156267 A1, incorporated herein by reference, as 5′-cap structures, cap analogues, RNA modifications, modified RNA, chemical modifications, backbone modifications, sugar modifications, base modifications, nucleotide analogues/modifications, or modified nucleotides.

RNA modifications of the present invention may include, but are not limited to, those described in US Patent Application Pub. No. US 2021/0260097, incorporated herein by reference, as modified mRNAs, mmRNAs, modified nucleobases, modified nucleosides, modified nucleotides, chemically modified mRNAs, or nucleoside modifications.

RNA modifications of the present invention may include, but are not limited to, those described in WO Patent Application Pub. No. WO 2021/213945 A1, incorporated herein by reference, as modified nucleosides, further modified nucleosides, modified nucleobases, modified nucleotides, 5′-cap, 5′ cap-analog, or capping structure at the 5′-end of the RNA.

In one embodiment one or more RNA substance may comprise an open reading frame. In one embodiment one or more RNA substance may not comprise an open reading frame. In one embodiment one or more RNA substance may comprise a 5′-cap or 5′-cap structure. In one embodiment one or more RNA substance may comprise a 5′ UTR. In one embodiment one or more RNA substance may comprise a 3′ UTR. In one embodiment one or more RNA substance may comprise a poly(A)-tail.

In one embodiment one or more RNA substance may be polymeric. In one embodiment one or more RNA substance may be single stranded. In one embodiment one or more RNA substance may be double stranded. In one embodiment one or more RNA substance may have one or more complimentary strands or partially complimentary strands, wherein a complimentary or partially complementary strand may include, but is not limited to, an RNA strand, DNA strand, peptide nucleic acid strand or other type of complementary or partially complementary strand.

In one embodiment one or more RNA substance may comprise a coding RNA. As a non-limiting example, a coding RNA may include, but is not limited to, mRNA or self-amplifying RNA.

In one embodiment one or more RNA substance may comprise a non-coding RNA. As a non-limiting example, a non-coding RNA may include, but is not limited to, microRNA, siRNA, CRISPR RNA, antisense RNA, small activating RNA, or RNA enzymes.

In one embodiment an RNA substance may be comprised of at least 2 or more nucleotides. In one embodiment an RNA substance may be comprised of at least 5 or more nucleotides. In one embodiment an RNA substance may be comprised of at least 10 or more nucleotides. In one embodiment an RNA substance may be comprised of at least 20 or more nucleotides. In one embodiment an RNA substance may be comprised of at least 50 or more nucleotides. In one embodiment an RNA substance may be comprised of at least 100 or more nucleotides. In one embodiment an RNA substance may be comprised of at least 200 or more nucleotides. In one embodiment an RNA substance may be comprised of at least 300 or more nucleotides. In one embodiment an RNA substance may be comprised of at least 400 or more nucleotides. In one embodiment an RNA substance may be comprised of at least 500 or more nucleotides. In one embodiment an RNA substance may be comprised of at least 600 or more nucleotides. In one embodiment an RNA substance may be comprised of at least 700 or more nucleotides. In one embodiment an RNA substance may be comprised of at least 800 or more nucleotides. In one embodiment an RNA substance may be comprised of at least 900 or more nucleotides. In one embodiment an RNA substance may be comprised of at least 1,000 or more nucleotides.

In one embodiment an RNA substance may be comprised of 2-1,000,000 nucleotides. In one embodiment an RNA substance may be comprised of 2-500,000 nucleotides. In one embodiment an RNA substance may be comprised of 2-100,000 nucleotides. In one embodiment an RNA substance may be comprised of 2-50,000 nucleotides. In one embodiment an RNA substance may be comprised of 2-20,000 nucleotides. In one embodiment an RNA substance may be comprised of 2-10,000 nucleotides.

In one embodiment an RNA substance may be comprised of 5-1,000,000 nucleotides. In one embodiment an RNA substance may be comprised of 5-500,000 nucleotides. In one embodiment an RNA substance may be comprised of 5-100,000 nucleotides. In one embodiment an RNA substance may be comprised of 5-50,000 nucleotides. In one embodiment an RNA substance may be comprised of 5-20,000 nucleotides. In one embodiment an RNA substance may be comprised of 5-10,000 nucleotides.

In one embodiment an RNA substance may be comprised of 10-1,000,000 nucleotides. In one embodiment an RNA substance may be comprised of 10-500,000 nucleotides. In one embodiment an RNA substance may be comprised of 10-100,000 nucleotides. In one embodiment an RNA substance may be comprised of 10-50,000 nucleotides. In one embodiment an RNA substance may be comprised of 10-20,000 nucleotides. In one embodiment an RNA substance may be comprised of 10-10,000 nucleotides.

In one embodiment an RNA substance may be comprised of 20-1,000,000 nucleotides. In one embodiment an RNA substance may be comprised of 20-500,000 nucleotides. In one embodiment an RNA substance may be comprised of 20-100,000 nucleotides. In one embodiment an RNA substance may be comprised of 20-50,000 nucleotides. In one embodiment an RNA substance may be comprised of 20-20,000 nucleotides. In one embodiment an RNA substance may be comprised of 20-10,000 nucleotides.

In one embodiment an RNA substance may be comprised of 100-1,000,000 nucleotides. In one embodiment an RNA substance may be comprised of 100-500,000 nucleotides. In one embodiment an RNA substance may be comprised of 100-100,000 nucleotides. In one embodiment an RNA substance may be comprised of 100-50,000 nucleotides. In one embodiment an RNA substance may be comprised of 100-20,000 nucleotides. In one embodiment an RNA substance may be comprised of 100-10,000 nucleotides.

Descriptions of substances herein may include one or more forms of the substance. These forms may include, but are not limited to, one more counterions, ionic forms, conjugate bases, conjugate acids, protonated or deprotonated forms, isomers, structural isomers, stereo isomers, chiral forms, salts, or combinations thereof.

As used herein, organic or organic substance means a substance comprised of at least one or more carbon atom, wherein at least one or more carbon atom is covalently bonded to at least one or more hydrogen atom.

Aprotic Substance

As used herein, an aprotic substance is an organic substance that is substantially incapable of donating or accepting hydrogen ions at one or more pH in the range of about physiologic pH. Therefore, an aprotic substance generally does not contribute to hydrogen ion exchange at one or more pH in the range of about physiologic pH.

As used herein, a polar aprotic substance is an aprotic substance comprised of at least one oxygen atom or at least one nitrogen atom.

As used herein, physiologic pH means pH in the range of about 5-9.

Low Dielectric Substance

As used herein, low dielectric substances are substances with a dielectric constant (also known as relative permittivity) at 20° C. under standard atmospheric pressure and 1 kHz that is less than about 80. Furthermore, a low dielectric substance is not a lipid, cholesterol, saccharide, polysaccharide, or starch.

In one embodiment a low dielectric substance may not be a detergent. In one embodiment a low dielectric substance may not be a peptide or polypeptide. In one embodiment a low dielectric substance may not be protein. In one embodiment a low dielectric substance may not be a nucleic acid base. In one embodiment a low dielectric substance may not be a nucleic acid.

Cyclic Phosphate

As used herein, a cyclic phosphate comprises one or more phosphate group wherein at least one phosphate group is at least part of a cyclic ring structure.

As used herein, a cyclic phosphate or metaphosphate containing substance is a substance that comprises at least one or more cyclic phosphate or one or more metaphosphate. Furthermore, a cyclic phosphate or metaphosphate containing substance is not a lipid, cholesterol, saccharide, polysaccharide, or starch.

In one embodiment a cyclic phosphate or metaphosphate containing substance may not be a detergent. In one embodiment a cyclic phosphate or metaphosphate containing substance may not be a peptide or polypeptide. In one embodiment a cyclic phosphate or metaphosphate containing substance may not be protein. In one embodiment a cyclic phosphate or metaphosphate containing substance may not be a nucleic acid base. In one embodiment a cyclic phosphate or metaphosphate containing substance may not be a nucleic acid.

Non-Carbohydrate Organic Osmolyte

As used herein, a non-carbohydrate organic osmolyte means a substance comprised of at least one of the following substances: amino acid, amino acid derivative, or low molecular weight aliphatic polyamine, wherein the substance comprises at least one carbon atom. Furthermore, a non-carbohydrate organic osmolyte substance is not a lipid, cholesterol, saccharide, polysaccharide, or starch.

In one embodiment a non-carbohydrate organic osmolyte substance may not be a detergent. In one embodiment a non-carbohydrate organic osmolyte substance may not be a peptide or polypeptide. In one embodiment a non-carbohydrate organic osmolyte substance may not be protein. In one embodiment a non-carbohydrate organic osmolyte substance may not be a nucleic acid base. In one embodiment a non-carbohydrate organic osmolyte substance may not be a nucleic acid.

Tertiary Sulfonium

As used herein, a tertiary sulfonium is a cationic sulfur atom that has at least three covalent bonds and is covalently bonded to at least three carbon atoms.

As used herein a tertiary sulfonium containing substance is a substance that comprises at least one tertiary sulfonium. Furthermore, a tertiary sulfonium containing substance is not a lipid, cholesterol, saccharide, polysaccharide, or starch.

In one embodiment a tertiary sulfonium containing substance may not be a detergent. In one embodiment a tertiary sulfonium containing substance may not be a peptide or polypeptide. In one embodiment a tertiary sulfonium containing substance may not be protein. In one embodiment a tertiary sulfonium containing substance may not be a nucleic acid base. In one embodiment a tertiary sulfonium containing substance may not be a nucleic acid.

Quaternary Ammonium

As used herein, a quaternary ammonium is a cationic nitrogen atom that has at least more than three covalent bonds and is covalently bonded to at least three or more carbon atoms.

As used herein a quaternary ammonium containing substance is a substance that comprises at least one quaternary ammonium. Furthermore, a quaternary ammonium containing substance is not a lipid, cholesterol, saccharide, polysaccharide, or starch.

In one embodiment a quaternary ammonium containing substance may not be a detergent. In one embodiment a quaternary ammonium containing substance may not be a peptide or polypeptide. In one embodiment a quaternary ammonium containing substance may not be protein. In one embodiment a quaternary ammonium containing substance may not be a nucleic acid base. In one embodiment a quaternary ammonium containing substance may not be a nucleic acid.

Quaternary Phosphonium

As used herein, a quaternary phosphonium is a phosphorus atom covalently bonded to at least three or more carbon atoms.

As used herein a quaternary phosphonium containing substance is a substance that comprises at least one quaternary phosphonium. Furthermore, a quaternary phosphonium containing substance is not a lipid, cholesterol, saccharide, polysaccharide, or starch.

In one embodiment a quaternary phosphonium containing substance may not be a detergent. In one embodiment a quaternary phosphonium containing substance may not be a peptide or polypeptide. In one embodiment a quaternary phosphonium containing substance may not be protein. In one embodiment a quaternary phosphonium containing substance may not be a nucleic acid base. In one embodiment a quaternary phosphonium containing substance may not be a nucleic acid.

Hydrotrope

As used herein, a hydrotrope is a non-micelle forming substance comprised of at least one five membered or six membered cyclic or heterocyclic ring structure and at least one oxygen atom or at least one nitrogen atom, wherein the at least one oxygen atom or at least one nitrogen atom may be at least part of the ring structure or may be attached to the ring structure as at least part of one or more functional groups. Furthermore, a hydrotrope is comprised of at least 5 carbon atoms.

As used herein a hydrotrope containing substance is a substance that comprises at least one hydrotrope. Furthermore, a hydrotrope containing substance is not a lipid, cholesterol, saccharide, polysaccharide, or starch.

In one embodiment a hydrotrope containing substance may not be a detergent. In one embodiment a hydrotrope containing substance may not be a peptide or polypeptide. In one embodiment a hydrotrope containing substance may not be protein. In one embodiment a hydrotrope containing substance may not be a nucleic acid base. In one embodiment a hydrotrope containing substance may not be a nucleic acid.

Surfactant

As used herein, a surfactant is a micelle forming substance comprised of at least one hydrophobic group, wherein a hydrophobic group is comprised of at least six or more aliphatic carbon atoms, and at least one hydrophilic group, wherein a hydrophilic group is comprised of at least one oxygen atom or at least one nitrogen atom.

As used herein a surfactant containing substance is a substance that comprises at least one surfactant. Furthermore, a surfactant containing substance is not a lipid, cholesterol, saccharide, polysaccharide, or starch.

In one embodiment a surfactant containing substance may be a detergent.

In one embodiment a surfactant containing substance may not be a peptide or polypeptide. In one embodiment a surfactant containing substance may not be protein. In one embodiment a surfactant containing substance may not be a nucleic acid base. In one embodiment a surfactant containing substance may not be a nucleic acid.

Betaine

As used herein, a betaine is a substance, wherein at about physiologic pH, is comprised of both a cationic moiety and an anionic moiety wherein the cationic moiety and the anionic moiety are separated by at least one carbon atom. Furthermore, the cationic moiety may comprise a quaternary ammonium cation or tertiary sulfonium cation as non-limiting examples. Meanwhile, the anionic moiety may comprise a carboxylate group, sulfonate group, organophosphate group, or organosulfate group as non-limiting examples.

As used herein a betaine containing substance is a substance that comprises at least one betaine. Furthermore, a betaine containing substance is not a lipid, cholesterol, saccharide, polysaccharide, or starch.

In one embodiment a betaine containing substance may comprise one or more cationic moiety, wherein a cationic moiety may comprise one or more quaternary ammonium cation or one or more tertiary sulfonium cation.

In one embodiment a betaine containing substance may comprise one or more anionic moiety, wherein an anionic moiety may comprise one or more carboxylate group, or one or more sulfonate group, or one or more organophosphate group, or one or more organosulfate group.

In one embodiment a betaine containing substance may not be a detergent. In one embodiment a betaine containing substance may not be a peptide or polypeptide. In one embodiment a betaine containing substance may not be protein. In one embodiment a betaine containing substance may not be a nucleic acid base. In one embodiment a betaine containing substance may not be a nucleic acid.

As used herein, NDSB means non-detergent sulfobetaine.

As used herein, an anion is an atom or group of atoms, with a negative charge at about physiologic pH.

As used herein, a cation is an atom or group of atoms, with a positive charge at about physiologic pH.

As used herein, a zwitterion is comprised of both a positively charged cationic moiety and a negatively charged anionic moiety at about physiologic pH.

As used herein, an organosulfate is comprised of a sulfate group, wherein at least one oxygen atom is covalently bonded to at least one carbon atom.

As used herein, an organophosphate is comprised of a phosphate group, wherein at least one oxygen atom is covalently bonded to a carbon atom. Furthermore, an organophosphate may be comprised of one oxygen atom of a phosphate group covalently bonded to a carbon atom or two oxygen atoms of a phosphate group each covalently bonded to different carbon atoms.

As used herein a combination of one or more RNA substances with one or more RNA stabilizing substances comprises one or more RNA substances and one or more RNA stabilizing substances and the combination may include one or more additional other substances. As a non-limiting example, a combination of at least one aprotic substance and at least one RNA substance comprises at least one aprotic substance and at least one RNA substance and other substances may also be parts of the combination.

### BRIEF DESCRIPTION OF THE DRAWINGS

For a more complete understanding of the present invention, and further advantages thereof, reference is now made to the following detailed description, taken in conjunction with the drawings, as described below.

FIG. 1 shows purified RNA following in vitro transcription analyzed by denaturing agarose gel electrophoresis. The PCR marker used is a double stranded DNA marker. RNA runs on denaturing agarose gel electrophoresis with an apparent molecular weight between about 1,000-1,500 bases. A black and white image of purified RNA as well as a grayscale image of purified RNA are shown.

FIG. 2 shows a comparison of RNA stability when stored in DMSO or Tris Acetate EDTA (TAE) (pH 8) over the course of about 280 days at various temperatures ranging from room temperature (RT) (about 20-25° C.), about 4° C., about −20° C., and about −80° C. in accordance with the present invention. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 3 shows a comparison of RNA stability when stored in varying concentrations of DMSO and 50 mM sodium acetate (pH 5.2) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 4 shows a comparison of RNA stability when stored in varying concentrations of N-Methyl-2-pyrrolidone (NMP) and 50 mM sodium acetate (pH 5.2) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 5 shows a comparison of RNA stability when stored in varying concentrations of sodium trimetaphosphate (TMP) and 50 mM sodium acetate (pH 5.2) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 6 shows a comparison of RNA stability when stored in varying concentrations of sodium hexametaphosphate (HMP) and 50 mM sodium acetate (pH 5.2) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 7 shows a comparison of RNA stability when stored in varying concentrations of sodium hexametaphosphate (HMP) and 50 mM Tris-HCl (pH 7) for about 48 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 8 shows a comparison of RNA stability when stored in varying concentrations of hexylene glycol and 50 mM sodium acetate (pH 5.2) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 9 shows a comparison of RNA stability when stored in varying concentrations of sodium glycerol phosphate and 50 mM sodium acetate (pH 5.2) for about 48 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 10 shows a comparison of RNA stability when stored in 1-butyl-1-methylpyrrolidinium bromide, benzyltriethylammonium chloride, or N,N-dimethylphenethylamine, and 50 mM sodium acetate (pH 5.2) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

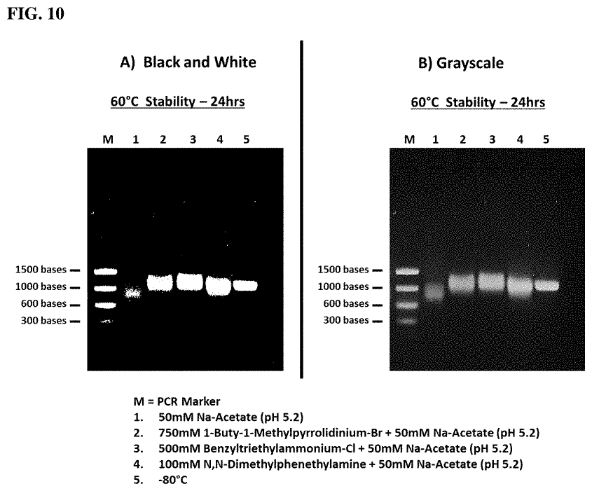

FIG. 11 shows a comparison of RNA stability when stored in varying concentrations of sodium benzoate and 50 mM sodium acetate (pH 5.2) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 12 shows a comparison of RNA stability when stored in varying concentrations of sodium benzoate with 1M trimethylglycine (TMG) and 50 mM Tris-HCl (pH 7) for about 48 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 13 shows a comparison of RNA stability when stored in varying concentrations of sodium benzoate with 50% DMSO and 50 mM Tris-HCl (pH 7) for about 48 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 14 shows a comparison of RNA stability when stored in varying concentrations of trimethyloctylammonium bromide and 50 mM sodium acetate (pH 5.2) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 15 shows a comparison of RNA stability when stored in quinolinic acid, nicotinamide N-oxide, nicotinic acid, or 1-methylnicotinamide chloride, and 50 mM sodium acetate (pH 5.2) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 16 shows a comparison of RNA stability when stored in ectoine, L-proline, glycine, or taurine, and 5 mM sodium phosphate (pH 8) and 10 mM sodium acetate (pH 7) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 17 shows a comparison of RNA stability when stored in ectoine, L-proline, glycine, or taurine, with 60% DMSO and 50 mM sodium acetate (pH 7) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 18 shows a comparison of RNA stability when stored in varying concentrations of dimethylsulfoniopropionate (DMSP) and TAE (pH 8) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 19 shows a comparison of RNA stability when stored in varying concentrations of DMSP with 50% DMSO and 50 mM sodium acetate (pH 7) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 20 shows a comparison of RNA stability when stored in varying concentrations of choline chloride and 50 mM sodium acetate (pH 5.2) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 21 shows a comparison of RNA stability when stored in varying concentrations of choline chloride and 50 mM Tris-HCl (pH 7) for about 24 hrs at 70° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 22 shows a comparison of RNA stability when stored in varying concentrations of choline chloride with 50% DMSO and 50 mM Tris-HCl (pH 7) for about 48 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 23 shows a comparison of RNA stability when stored in varying concentrations of acetylcholine chloride and 50 mM sodium acetate (pH 5.2) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 24 shows a comparison of RNA stability when stored in varying concentrations of acetylcholine chloride and 5 mM sodium phosphate (pH 8) and 10 mM sodium acetate (pH 7) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 25 shows a comparison of RNA stability when stored in varying concentrations of acetylcholine chloride with 50% DMSO and 50 mM sodium acetate (pH 7) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 26 shows a comparison of RNA stability when stored in trimethylglycine (TMG), NDSB-195, NDSB-221, or L-carnitine, and 5 mM sodium phosphate (pH 8) and 10 mM sodium acetate (pH 7) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 27 shows a comparison of RNA stability when stored in TMG, NDSB-195, NDSB-201, NDSB-221, L-carnitine, stachydrine, or L-alpha-glycerylphosphorylcholine (alpha-GPC), with 60% DMSO and 50 mM sodium acetate (pH 7) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 28 shows a comparison of RNA stability when stored in varying concentrations of ˜8.5 kDa poly(2-(trimethylamino)ethyl methacrylate) chloride (PTMAEMA) with 50 mM Tris-HCl (pH 7) over the course of about 24 hrs at 70° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 29 shows a comparison of RNA stability when stored in varying concentrations of ˜8.5 kDa poly(diallyldimethylammonium chloride) (PDADMAC) with 50 mM Tris-HCl (pH 7) over the course of about 24 hrs at 70° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 30 shows a comparison of RNA stability when stored in varying concentrations of ˜10 kDa poly(2-(diethylamino)ethyl methacrylate) (PDEAEMA) and 50 mM sodium acetate (pH 5.2) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

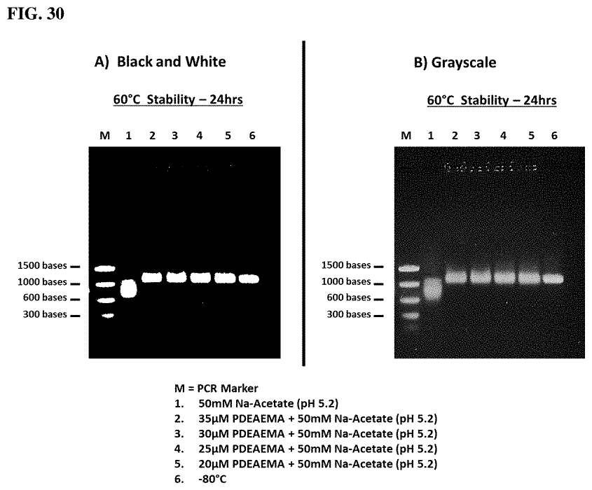

FIG. 31 shows a comparison of RNA stability when stored in varying concentrations of ˜7.5 kDa poly(2-(N-3-sulfopropyl-N,N-dimethyl ammonium)ethyl methacrylate) (PSBMA) with 100 mM sodium benzoate and 5 mM sodium phosphate (pH 8) and 10 mM sodium acetate (pH 7) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 32 shows a comparison of RNA stability when stored in varying concentrations of ˜9 kDa poly(2-methacryloyloxyethyl phosphorylcholine) (PMPC) with 100 mM sodium benzoate and 5 mM sodium phosphate (pH 8) and 10 mM sodium acetate (pH 7) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 33 shows a comparison of RNA stability when stored in varying concentrations of both ˜7.5 kDa PSBMA and ˜9 kDa PMPC with 100 mM sodium benzoate and 5 mM sodium phosphate (pH 8) and 10 mM sodium acetate (pH 7) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 34 shows a comparison of RNA stability when stored in ˜7.5 kDa PSBMA, ˜9 kDa PMPC, a combination of both ˜7.5 kDa PSBMA and ˜9 kDa PMPC, PEG-block-PSBMA block copolymer (PEG-PSBMA) (PEG Mₙ 5,000; PSBMA Mₙ 13,000), PEG-block-PMPC block copolymer (PEG-PMPC) (PEG Mₙ 5,000; PMPC Mₙ 21,000), or ˜10 kDa polyvinylpyrrolidone (PVP), with 100 mM sodium benzoate and 5 mM sodium phosphate (pH 8) and 10 mM sodium acetate (pH 7) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 35 shows a comparison of RNA stability when stored in varying concentrations of ˜8 kDa poly(acrylic acid, sodium salt) (PAA) and 50 mM sodium acetate (pH 5.2) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 36 shows a comparison of RNA stability when stored in ˜7.5 kDa PSBMA, ˜9 kDa PMPC, a combination of both ˜7.5 kDa PSBMA and ˜9 kDa PMPC, PEG-PSBMA (PEG Mₙ 5,000; PSBMA Mₙ 13,000), PEG-PMPC (PEG Mₙ 5,000; PMPC Mₙ 21,000), poly(ethylene glycol) 8,000 (PEG), poly(propylene glycol) 425 (PPG) (Mₙ ˜425), or ˜10 kDa PVP, with 50 μM ˜8.5 kDa PTMAEMA and 50 mM sodium acetate (pH 5.2) for about 48 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 37 shows a comparison of RNA stability when stored in ˜7.5 kDa PSBMA, ˜9 kDa PMPC, a combination of both ˜7.5 kDa PSBMA and ˜9 kDa PMPC, PEG-PSBMA (PEG Mₙ 5,000; PSBMA Mₙ 13,000), PEG-PMPC (PEG Mₙ 5,000; PMPC Mₙ 21,000), PEG 8,000, PPG 425, or ˜10 kDa PVP, with 100 μM ˜8.5 kDa PDADMAC and 50 mM sodium acetate (pH 5.2) for about 48 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 38 shows a comparison of RNA stability when stored in compositions comprising various combinations of more than one RNA stabilizing substance and 50 mM sodium acetate (pH 5.2) for about 24 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 39 shows a comparison of RNA stability when stored in compositions comprising various combinations of more than one RNA stabilizing substance and 50 mM sodium acetate (pH 5.2) for about 48 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 40 shows a comparison of RNA stability when stored in compositions comprising various combinations of more than one RNA stabilizing substance and 50 mM Tris-HCl (pH 7) for about 48 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

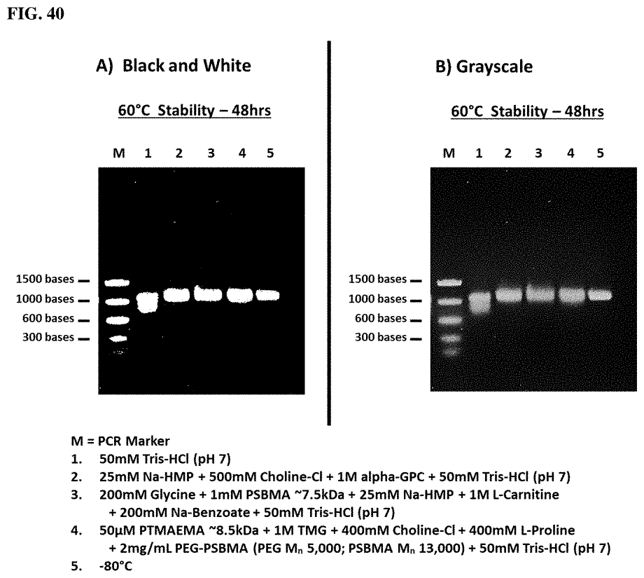

FIG. 41 shows a comparison of RNA stability when stored in compositions comprising various combinations of more than one RNA stabilizing substance and 50 mM Tris-HCl (pH 7) for about 72 hrs at 60° C. in accordance with the present invention. A sample stored at −80° C. was run for reference. RNA degradation is indicated by changes in RNA apparent molecular weight, band sharpness, and band fluorescence intensity following denaturing agarose gel electrophoresis. Full length RNA has an apparent molecular weight between about 1,000-1,500 bases. A black and white image and a grayscale image are shown.

FIG. 42 shows non-limiting examples of an NPS stabilizing moiety in accordance with the present invention.

FIG. 43 shows non-limiting examples displaying how NPS stabilizing moieties may be comprised of heterocyclic five membered ring structures in accordance with the present invention.

FIG. 44 shows non-limiting examples displaying how NPS stabilizing moieties may be comprised of heterocyclic six membered ring structures in accordance with the present invention.

FIG. 45 shows non-limiting examples of COPS stabilizing moieties comprised of a carbon atom, phosphorus atom, or sulfur atom in accordance with the present invention.

FIG. 46 shows a non-limiting example diagram of a stabilizing monomer that may comprise an NPS stabilizing moiety and a COPS stabilizing moiety connected by a bridge R_{B}, in accordance with the present invention.

FIG. 47 shows non-limiting examples of stabilizing monomers comprised of an NPS stabilizing moiety and a COPS stabilizing moiety connected by a bridge R_{B}, in accordance with the present invention.

FIG. 48 shows non-limiting examples of stabilizing monomers comprised of an NPS stabilizing moiety and a COPS stabilizing moiety connected by a bridge, represented by an R group R_{B}, in accordance with the present invention.

FIG. 49 shows non-limiting example structures of stabilizing monomers comprised of an NPS stabilizing moiety and COPS stabilizing moiety connected by a bridge represented by R_{B} in accordance with the present invention.

FIG. 50 shows non-limiting example structures of stabilizing monomers comprised of an NPS stabilizing moiety and COPS stabilizing moiety connected by a bridge represented by R_{B} in accordance with the present invention.

FIG. 51 shows non-limiting example structures of stabilizing monomers comprising a heterocyclic NPS stabilizing moiety and a COPS stabilizing moiety in accordance with the present invention.

FIG. 52 shows non-limiting examples of stabilizing monomers comprised of an NPS stabilizing moiety and a COPS stabilizing moiety in accordance with the present invention.

FIG. 53 shows non-limiting examples of backbone repeat units that may form a backbone chain of a stabilizing polymer in accordance with the present invention.

FIG. 54 shows non-limiting examples of a stabilizing polymer assembly in accordance with the present invention.

FIG. 55 shows non-limiting examples of stabilizing polymers comprised of one or more stabilizing monomer in accordance with the present invention.

FIG. 56 shows non-limiting examples of stabilizing polymers comprised of one or more stabilizing monomer in accordance with the present invention.

FIG. 57 shows a multi-chamber syringe loaded with components of an RNA composition in accordance with the present invention.

FIG. 58 shows an RNA composition kit in accordance with the present invention.

FIG. 59 shows an RNA composition kit in accordance with the present invention

FIG. 60 shows an RNA composition kit in accordance with the present invention

FIG. 61 shows an RNA storage package in accordance with the present invention

FIG. 62 is a flow chart illustrating a process for producing and using an RNA product in accordance with the present invention.

FIG. 63 is a flow chart illustrating a process for producing and using an RNA product in accordance with the present invention.

FIG. 64 shows a vial container with RNA substance and RNA stabilizing substance in accordance with the present invention.

FIG. 65 shows a single chamber syringe container with RNA substance and RNA stabilizing substance in accordance with the present invention.

FIG. 66 shows an embedded complex container with RNA substance and RNA stabilizing substance in accordance with the present invention.

### DETAILED DESCRIPTION OF THE INVENTION

Before explaining various embodiments of RNA stabilizing substances, and storage environments of RNA substances in detail, it should be noted that the illustrative embodiments and examples are not limited in application or use to the details of construction and arrangement of parts illustrated in the accompanying drawings and description. The illustrative embodiments and examples may be implemented or incorporated in other embodiments, variations and modifications, and may be practiced or carried out in various ways. Further, unless otherwise indicated, the terms and expressions employed herein have been chosen for the purpose of describing the illustrative embodiments for the convenience of the reader and are not for the purpose of limitation thereof. Also, it will be appreciated that one or more of the following-described embodiments, expressions of embodiments and/or examples, may be combined with one or more of the other following-described embodiments, expressions of embodiments and/or examples.

As used herein the term “or” means and/or unless the context states otherwise.

As used herein the term “a” means one or more unless the context states otherwise.

As used herein the term “an” means one or more unless the context states otherwise.

It is of great interest to the field of therapeutics, diagnostics, and agriculture to increase the stability of RNA substances and reduce RNA degradation of RNA substances. Described herein, are compositions (including pharmaceutical compositions and therapeutic compositions) and methods for the design, preparation, manufacture and/or formulation of storage environments to stabilize RNA substances. Also provided are systems, processes, and devices for selection, design, and/or utilization of the storage environments to stabilize RNA substances described herein.

RNA is naturally unstable, including in aqueous solutions. RNA is susceptible to degradation including, but not limited to the following types of degradation: enzymatic, autocatalytic, metal-catalyzed, autohydrolysis, hydrolysis, temperature induced, pH induced, chemically induced, oxidation induced, reduction induced, or radiation induced. Without being bound to any particular mechanism or mode of action, it is believed that the present invention provides a storage environment that increases the stability of RNA substances at warmer than extreme cold conditions by reducing the exposure of RNA substances to substances within the storage environment that may induce RNA degradation.

The present inventors have discovered that RNA stabilizing substances can surprisingly increase the stability of RNA substances at temperatures above about −80° C. The present inventors have also discovered that RNA stabilizing substances can surprisingly increase the stability of RNA substances at temperatures above about −20° C. The present inventors have also discovered that RNA stabilizing substances can surprisingly increase the stability of RNA substances at temperatures above about 0° C. The present inventors have also discovered that RNA stabilizing substances can surprisingly increase the stability of RNA substances at temperatures above about 4° C. The present inventors have also discovered that RNA stabilizing substances can surprisingly increase the stability of RNA substances at temperatures above about 10° C. The present inventors have also discovered that RNA stabilizing substances can surprisingly increase the stability of RNA substances at temperatures above about 20° C.

The present inventors have discovered that compositions comprising at least one or more RNA stabilizing substances and at least one or more RNA substance may lead to increases in RNA stability compared to compositions containing at least one RNA substance without at least one or more RNA stabilizing substances.

Embodiments of the present invention may comprise one or more RNA stabilizing substances. Embodiments of the present invention may include compositions comprising one or more RNA stabilizing substance.

Embodiments of the present invention may include compositions comprised of one or more RNA stabilizing substance and one or more nucleic acid substance.

Embodiments of the present invention may include compositions comprised of one or more RNA stabilizing substance and one or more DNA substance.

The inventors have discovered that compositions comprising at least one or more RNA substance and at least one or more RNA stabilizing substance may improve RNA stability. The inventors have discovered that RNA stability may be improved in compositions comprising at least one or more RNA substance and multiple RNA stabilizing substances in which the number of multiple RNA substances may be two or more. As a non-limiting example, RNA stability may be improved in compositions comprising at least one or more RNA substance and multiple RNA stabilizing substances where the number of multiple RNA stabilizing substances is five.

Embodiments of the present invention may include compositions comprised of one or more RNA stabilizing substance and one or more RNA substance.

An embodiment of the present invention may include a composition that may comprise at least one or more RNA substance and at least one or more RNA stabilizing substance. Another embodiment of the present invention may include a composition that may comprise at least one RNA substance and two or more RNA stabilizing substances in which the composition may comprise two or more RNA stabilizing substances. Another embodiment of the present invention may include a composition that may comprise at least one RNA substance and three or more RNA stabilizing substances in which the composition may comprise three or more RNA stabilizing substances. Another embodiment of the present invention may include a composition that may comprise at least one RNA substance and four or more RNA stabilizing substances in which the composition may comprise four or more RNA stabilizing substances. Another embodiment of the present invention may include a composition that may comprise at least one RNA substance and five or more RNA stabilizing substances in which the composition may comprise five or more RNA stabilizing substances.

An embodiment of the present invention include a composition that may comprise at least one or more RNA substance and at least one or more RNA stabilizing substance that improves RNA stability. The inventors have discovered that RNA stability may be improved with compositions comprising at least one or more RNA substance and multiple RNA stabilizing substances in which the number of multiple RNA stabilizing substances may be between two and five, or greater than five. As a non-limiting example, RNA stability may be improved with compositions comprising at least one or more RNA substance and multiple RNA stabilizing substances where the number of multiple RNA stabilizing substances may be two RNA stabilizing substances, or three RNA stabilizing substances, or five RNA stabilizing substances, or more.

The storage environment that improves the stability of RNA substances may comprise at least one or more vapor, liquid, powder, or solid RNA stabilizing substance.

As described herein, the present inventors have discovered that selected materials may be used individually as RNA stabilizing substances and that selected materials may be used in combinations as RNA stabilizing substances.

The inventors have discovered previously unrecognized substances stabilize RNA or RNA substances.

The inventors have discovered that the following materials may be RNA stabilizing substances: aprotic substances, low dielectric substances, cyclic phosphate or metaphosphate containing substances, non-carbohydrate organic osmolyte substances, tertiary sulfonium containing substances, quaternary ammonium containing substances, quaternary phosphonium containing substances, hydrotrope containing substances, surfactant containing substances, betaine containing substances, stabilizing monomers, stabilizing polymers, or supplemental RNA stabilizing substances. Embodiments of the present invention may comprise one or more of these RNA stabilizing substances. Other embodiments of the present invention may comprise one or more RNA substance and one or more substance from one or more of the above categories of RNA stabilizing substances.

As nonlimiting examples, embodiments of the present invention may include combinations comprising one or more aprotic substances, one or more low dielectric substances, one or more cyclic phosphate or metaphosphate containing substances, one or more polyhydroxy or polyol containing substances, one or more non-carbohydrate organic osmolyte substances, one or more quaternary ammonium containing substances, one or more quaternary phosphonium containing substances, one or more tertiary sulfonium containing substances, one or more hydrotrope containing substances, one or more surfactant containing substances, one or more betaine containing substances, one or more stabilizing monomers, or one or more stabilizing polymers.

RNA stabilizing substances may take on a variety of ionic forms at about physiologic pH. For conciseness, the various ionic forms that RNA stabilizing substances may have, are herein called PIFs for possible ionic forms. For example, stabilizing monomers or stabilizing polymers may comprise one or more PIF. As a non-limiting example, RNA stabilizing substances at about physiologic pH may comprise one or more cationic moiety or one or more anionic moiety. As another non-limiting example RNA stabilizing substances at about physiologic pH may not comprise a cationic moiety or an anionic moiety. As another non-limiting example an RNA stabilizing substance at about physiologic pH may comprise one or more zwitterion or may not comprise a zwitterion. As another non-limiting example an RNA stabilizing substance at about physiologic pH may have a net positive charge, a net negative charge, or a net neutral charge. As another non-limiting example an RNA stabilizing substance at about physiologic pH may comprise both an anionic moiety and a cationic moiety. As another non-limiting example an RNA stabilizing substance at about physiologic pH may be anionic, or may be cationic, or may be zwitterionic.

Embodiments of the present invention may comprise one or more RNA stabilizing substance. Other embodiments of the present invention may comprise one or more RNA substance and one or more RNA stabilizing substance.

The inventors have discovered that RNA stabilizing substances may stabilize RNA substances.

The inventors have also discovered that the stability of RNA substances may be enhanced in an RNA storage environment comprising one or more RNA stabilizing substance.

The inventors have also discovered that the stability of RNA substances may be enhanced in compositions comprising an RNA substance and one or more RNA stabilizing substance.

One embodiment of the present invention may include a composition comprised of one or more RNA substance and one or more RNA stabilizing substance.

One embodiment of the present invention may include a combination or mixture comprising one or more RNA substance and one or more RNA stabilizing substance.

Embodiments of the present invention that comprise one or more RNA substance and one or more RNA stabilizing substance may include combining, such as by mixing, one or more RNA substance with one or more substance that comprises at least one or more RNA stabilizing substance.

An embodiment of the present invention may include compositions of materials that comprise one or more RNA substance and at least one or more RNA stabilizing substance. The storage environment that improves the stability of RNA substances may comprise at least one or more vapor, liquid, powder, or solid RNA stabilizing substance.

An embodiment of the present invention may include combinations of materials that comprise one or more RNA substance and at least one or more RNA stabilizing substance. The storage environment that improves the stability of RNA substances may comprise at least one or more vapor, liquid, powder, or solid RNA stabilizing substance.

In one embodiment of the present invention a composition comprised of one or more RNA substance and one or more RNA stabilizing substance, produces a mixture with at least one or more RNA substance and at least one or more RNA stabilizing substance.

In one embodiment of the present invention a combination comprising one or more RNA substance and one or more RNA stabilizing substance, produces a mixture with at least one or more RNA substance and at least one or more RNA stabilizing substance.

In one embodiment of the present invention a composition with improved RNA stability may comprise one or more RNA substance and one or more RNA stabilizing substance.

In one embodiment of the present invention a composition with improved RNA stability may comprise a mixture of one or more RNA substance and one or more RNA stabilizing substance.

In one embodiment of the present invention, each component included in a composition comprising one or more RNA substance and one or more RNA stabilizing substance, may be stored separately, such as in a kit, or such as individual substances or as mixtures of one or more substance, and then combined later to produce a composition comprising one or more RNA substance and one or more RNA stabilizing substance.

In one embodiment of the present invention a composition comprising one or more RNA substance and one or more RNA stabilizing substance may be at least partially biocompatible.

In one embodiment of the present invention a combination comprised of one or more RNA substance and one or more RNA stabilizing substance may be at least partially biocompatible.

Embodiments of the present invention may comprise one or more RNA stabilizing substance. Other embodiments of the present invention may comprise one or more RNA substance and one or more RNA stabilizing substance.

The inventors have discovered that RNA stabilizing substances may improve the stability of RNA substances in the presence of water. Embodiments of the present invention may include compositions that may comprise at least one RNA stabilizing substance, at least one RNA substance, and water. These embodiments that may comprise water may be one or more composition as described herein that may also comprise water.

Embodiments of the present invention may comprise one or more composition described herein and may include one or more additional other substances of which water may be one of the additional other substances.

Embodiments of the present invention may comprise one or more composition described herein and one or more cellular uptake agent. Such embodiments may be used as at least part of one or more therapeutic substances, pharmaceutical compositions, medicaments, vaccines, or biostimulants, such as, as a non-limiting example, vaccines deploying mRNA to one or more living organisms (which may include humans or may include non-human animals) with at least one RNA stabilizing substance improving the stability of the therapeutic substance and at least one cellular uptake agent improving the efficacy of the therapeutic substance.

Embodiments of the present invention may comprise one or more composition described herein and may also include one or more additional other substance, such as one or more cellular uptake agent, or one more additional RNA stabilizing substance, or one or more buffering agent, or one or more inorganic salt, or one or more chelating agent, or water as non-limiting examples. Such embodiments may be used as at least part of therapeutic substances, pharmaceutical compositions, medicaments, vaccines, or biostimulants, such as, as a non-limiting example, vaccines deploying mRNA to one or more living organisms (which may include humans or may include non-human animals) with at least one RNA stabilizing substance improving the stability of the therapeutic substance and at least one cellular uptake agent improving the efficacy of the therapeutic substance.

### Mechanisms and Theory

Without being bound to any particular mechanism or mode of action, RNA hydrolysis can be initiated by a base removing a proton from the 2′-OH on the ribose sugar, leading to the subsequent nucleophilic attack of the 2′ oxygen on the adjacent phosphorus atom. Base catalyzed hydrolysis activates the 2′-OH by removing the proton and creating a negatively charged 2′ oxygen and promoting nucleophilic attack of the 2′ oxygen on the adjacent phosphorus atom of the phosphodiester backbone of RNA. Water's protic nature to both donate and accept protons allows water to act as both an acid and a base at about physiologic pH. Therefore, water is capable of acting as a proton acceptor and activating the 2′-OH to promote the nucleophilic attack of the 2′ oxygen on the adjacent phosphorus atom of the phosphodiester backbone of the RNA molecule to promote RNA hydrolysis.

Without being bound to any particular mechanism or mode of action, aprotic substances are substantially incapable of donating or accepting hydrogen ions at one or more pH in the range of about physiologic pH, and, thus, generally do not contribute to hydrogen ion exchange that may promote hydrolysis of RNA substances. Creating an environment with substantially less hydrogen ion exchange, such as in the presence of an aprotic substance, then the initiation of RNA hydrolysis and subsequent cleavage of the RNA molecule will be less favorable. Therefore, aprotic substances do not promote the nucleophilic attack of the 2′-OH on the adjacent phosphorus atom of the phosphodiester backbone, such as occurs in the presence of water. Thus, by making initiation of RNA hydrolysis less favorable, aprotic substances may be RNA stabilizing substances.

Without being bound to any particular mechanism or mode of action, one or more RNA stabilizing substance may at least reduce access to the RNA substance by materials that may promote RNA hydrolysis or degradation of the RNA substance. In a non-limiting example, one or more RNA stabilizing substance in the environment of the RNA substance may create an environment that excludes water from the RNA substance or reduces the concentration of water in the environment around the RNA substance or alters the water structure or hydrogen bonding network of water or the environment around the RNA substance. Therefore, if one or more RNA stabilizing substances substantially displace all of the water in the environment of the RNA substance then the RNA substance is substantially not exposed to water, ions, or other materials that may promote RNA hydrolysis. In another non-limiting example, one or more RNA stabilizing substance in the environment of the RNA substance may also create an environment that limits the molecular mobility of water, ions, or other materials and thereby limit and/or prevent the exposure of the RNA substance to water, ions, or other materials that may promote RNA hydrolysis.

Double stranded RNA substances are more stable than single stranded RNA substances. Without being bound to any particular mechanism or mode of action, the increased stability of double stranded RNA is at least partially a result of the decreased flexibility of the double stranded RNA substance which reduces the movement of the RNA substance creating a lower probability that a 2′-OH will be in close enough proximity to an adjacent phosphorus atom to perform a nucleophilic attack and initiate RNA hydrolysis. In a non-limiting example, one or more RNA stabilizing substance that reduces the flexibility or molecular movement of a single stranded RNA substance reduces the likelihood that a 2′-OH will be in close enough proximity to an adjacent phosphorus atom to perform a nucleophilic attack and initiate RNA hydrolysis.

Embodiments of the present invention that comprise an RNA substance and at least one or more RNA stabilizing substance may include combining, such as by mixing, at least one RNA substance with at least one substance that comprises at least one or more RNA stabilizing substance. Without being bound to any particular mechanism or mode of action, one or more RNA stabilizing substance may at least partially reduce access to the RNA substance by materials that may promote hydrolysis or degradation of the RNA substance. In a non-limiting example, one or more RNA stabilizing substance in the environment of the RNA substance may reduce the concentration of water in the environment around the RNA substance. In another non-limiting example, if one or more RNA stabilizing substances substantially displace all of the water in the environment of the RNA substance then the RNA substance is substantially not exposed to water or other materials that may promote hydrolysis.

Embodiments of the present invention comprising at least one or more RNA stabilizing substance may include one or more forms of an RNA stabilizing substance. These forms may include, but are not limited to, one more counterions, ionic forms, conjugate bases, conjugate acids, protonated or deprotonated forms, isomers, structural isomers, stereo isomers, chiral forms, salts, or combinations thereof.

### Aprotic Substances:

The inventors have discovered that aprotic substances may stabilize RNA substances.

The inventors have discovered that RNA stabilizing substances may comprise aprotic substances. The inventors have also discovered that the stability of RNA substances may be enhanced in an RNA storage environment comprising one or more aprotic substance.

The inventors have also discovered that the stability of RNA substances may be enhanced in compositions comprising an RNA substance and one or more aprotic substance.

In one embodiment of the present invention, one or more RNA stabilizing substance may comprise one or more aprotic substance.

One embodiment of the present invention may include a composition comprised of one or more RNA substance and one or more aprotic substance.

One embodiment of the present invention may include a combination or mixture comprising one or more RNA substance and one or more aprotic substance.

Embodiments of the present invention that comprise one or more RNA substance and one or more aprotic substance may include combining, such as by mixing, one or more RNA substance with one or more substance that comprises at least one or more aprotic substance.

An embodiment of the present invention may include compositions of materials that comprise one or more RNA substance and at least one or more aprotic substance. The storage environment that improves the stability of RNA substances may comprise at least one or more vapor, liquid, powder, or solid aprotic substance.

An embodiment of the present invention may include combinations of materials that comprise one or more RNA substance and at least one or more aprotic substance. The storage environment that improves the stability of RNA substances may comprise at least one or more vapor, liquid, powder, or solid aprotic substance.

In one embodiment of the present invention a composition comprised of one or more RNA substance and one or more aprotic substance, produces a mixture with at least one or more RNA substance and at least one or more aprotic substance.

In one embodiment of the present invention a combination comprising one or more RNA substance and one or more aprotic substance, produces a mixture with at least one or more RNA substance and at least one or more aprotic substance.

In one embodiment of the present invention a composition with improved RNA stability may comprise one or more RNA substance and one or more aprotic substance.

In one embodiment of the present invention a composition with improved RNA stability may comprise a mixture of one or more RNA substance and one or more aprotic substance.

In one embodiment of the present invention, each component included in a composition comprising one or more RNA substance and one or more aprotic substance, may be stored separately, such as in a kit, or such as individual substances or as mixtures of one or more substance, and then combined later to produce a composition comprising one or more RNA substance and one or more aprotic substance.

In one embodiment of the present invention a composition comprising one or more RNA substance and one or more aprotic substance may be at least partially biocompatible.

In one embodiment of the present invention a combination comprised of one or more RNA substance and one or more aprotic substance may be at least partially biocompatible.

Embodiments of the present invention comprising at least one or more aprotic substance may include one or more forms of the aprotic substance. These forms may include, but are not limited to, isomers, structural isomers, stereo isomers, chiral forms, salts, or combinations thereof.

A non-limiting example of an aprotic substance is dimethyl sulfoxide (DMSO). One embodiment of the present invention may include combinations of substances comprising DMSO and another embodiment may also comprise at least one RNA substance.

Embodiments of compositions comprising one or more RNA stabilizing substance, such as an aprotic substance of which DMSO is a non-limiting example, may have different weight percent concentrations. For conciseness, the following list of weight percent concentrations, with DMSO as a non-limiting example aprotic substance, is herein referred to as the stabilizing substance weight percent concentration list, and herein also referred to as the RNA stabilizing substance weight percent concentration list.

In one embodiment the concentration of DMSO may be at least 0.0001 percent (all composition percentages herein are weight percent, unless stated otherwise) of the substances that are not RNA substances. In one embodiment the concentration of DMSO may be at least 0.001 percent of the substances that are not RNA substances. In one embodiment the concentration of DMSO may be at least 0.01 percent of the substances that are not RNA substances. In one embodiment the concentration of DMSO may be at least 0.1 percent of the substances that are not RNA substances. In one embodiment the concentration of DMSO may be at least 1 percent of the substances that are not RNA substances. In one embodiment the concentration of DMSO may be at least 2 percent of the substances that are not RNA substances. In one embodiment the concentration of DMSO may be at least 3 percent of the substances that are not RNA substances. In one embodiment the concentration of DMSO may be at least 4 percent of the substances that are not RNA substances. In one embodiment the concentration of DMSO may be at least 5 percent of the substances that are not RNA substances. In one embodiment the concentration of DMSO may be at least 10 percent of the substances that are not RNA substances. In one embodiment the concentration of DMSO may be at least 15 percent of the substances that are not RNA substances. In one embodiment the concentration of DMSO may be at least 20 percent of the substances that are not RNA substances. In one embodiment the concentration of DMSO may be at least 25 percent of the substances that are not RNA substances. In one embodiment the concentration of DMSO may be at least 30 percent of the substances that are not RNA substances. In one embodiment the concentration of DMSO may be at least 35 percent of the substances that are not RNA substances. In one embodiment the concentration of DMSO may be at least 40 percent of the substances that are not RNA substances. In one embodiment the concentration of DMSO may be at least 45 percent of the substances that are not RNA substances. In one embodiment the concentration of DMSO may be at least 50 percent of the substances that are not RNA substances. In one embodiment the concentration of DMSO may be at least 55 percent of the substances that are not RNA substances. In one embodiment the concentration of DMSO may be at least 60 percent of the substances that are not RNA substances. In one embodiment the concentration of DMSO may be at least 65 percent of the substances that are not RNA substances. In one embodiment the concentration of DMSO may be at least 70 percent of the substances that are not RNA substances. In one embodiment the concentration of DMSO may be at least 75 percent of the substances that are not RNA substances. In one embodiment the concentration of DMSO may be at least 80 percent of the substances that are not RNA substances. In one embodiment the concentration of DMSO may be at least 85 percent of the substances that are not RNA substances. In one embodiment the concentration of DMSO may be at least 90 percent of the substances that are not RNA substances. In one embodiment the concentration of DMSO may be at least 95 percent of the substances that are not RNA substances. In one embodiment the concentration of DMSO may be at least 99 percent or even substantially all of the substances that are not RNA substances.

A non-limiting example of an aprotic substance is dimethyl sulfone (also known as DMSO₂, methylsulfonylmethane, MSM, or methyl sulfone). One embodiment of the present invention may include combinations of substances comprising dimethyl sulfone and another embodiment may also comprise at least one RNA substance.

Embodiments of compositions comprising one or more RNA stabilizing substance, such as an aprotic substance of which dimethyl sulfone is a non-limiting example, may have different molar concentrations. For conciseness, the following list of molar concentrations, with dimethyl sulfone as a non-limiting example aprotic substance, is herein referred to as the stabilizing substance molar concentration range list, and herein also referred to as the RNA stabilizing substance molar concentration range list.

In one embodiment the concentration of dimethyl sulfone may be between about 1 nM-10M. In one embodiment the concentration of dimethyl sulfone may be between about 1 nM-9.5M. In one embodiment the concentration of dimethyl sulfone may be between about 1 nM-9M. In one embodiment the concentration of dimethyl sulfone may be between about 1 nM-8.5M. In one embodiment the concentration of dimethyl sulfone may be between about 1 nM-8M. In one embodiment the concentration of dimethyl sulfone may be between about 1 nM-7.5M. In one embodiment the concentration of dimethyl sulfone may be between about 1 nM-7M. In one embodiment the concentration of dimethyl sulfone may be between about 1 nM-6.5M. In one embodiment the concentration of dimethyl sulfone may be between about 1 nM-6M. In one embodiment the concentration of dimethyl sulfone may be between about 1 nM-5.5M. In one embodiment the concentration of dimethyl sulfone may be between about 1 nM-5M. In another embodiment the concentration of dimethyl sulfone may be between about 1 nM-4.5M. In another embodiment the concentration of dimethyl sulfone may be between about 1 nM-4M. In another embodiment the concentration of dimethyl sulfone may be between about 1 nM-3.5M. In another embodiment the concentration of dimethyl sulfone may be between about 1 nM-3M. In another embodiment the concentration of dimethyl sulfone may be between about 1 nM-2.5M. In another embodiment the concentration of dimethyl sulfone may be between about 1 nM-2M. In another embodiment the concentration of dimethyl sulfone may be between about 1 nM-1.5M. In another embodiment the concentration of dimethyl sulfone may be between about 1 nM-1M. In another embodiment the concentration of dimethyl sulfone may be between about 1 nM-500 mM. In another embodiment the concentration of dimethyl sulfone may be between about 1 nM-250 mM. In another embodiment the concentration of dimethyl sulfone may be between about 1 nM-100 mM. In another embodiment the concentration of dimethyl sulfone may be between about 1 nM-50 mM. In another embodiment the concentration of dimethyl sulfone may be between about 1 nM-25 mM. In another embodiment the concentration of dimethyl sulfone may be between about 1 nM-10 mM. In another embodiment the concentration of dimethyl sulfone may be between about 1 nM-5 mM. In another embodiment the concentration of dimethyl sulfone may be between about 1 nM-1 mM. In another embodiment the concentration of dimethyl sulfone may be between about 1 nM-500 μM. In another embodiment the concentration of dimethyl sulfone may be between about 1 nM-250 μM. In another embodiment the concentration of dimethyl sulfone may be between about 1 nM-100 μM. In another embodiment the concentration of dimethyl sulfone may be between about 1 nM-50 μM. In another embodiment the concentration of dimethyl sulfone may be between about 1 nM-25 μM. In another embodiment the concentration of dimethyl sulfone may be between about 1 nM-10 μM. In another embodiment the concentration of dimethyl sulfone may be between about 1 nM-5 μM. In another embodiment the concentration of dimethyl sulfone may be between about 1 nM-1 μM.

In one embodiment the concentration of dimethyl sulfone may be between about 1 μM-10M. In one embodiment the concentration of dimethyl sulfone may be between about 1 μM-9.5M. In one embodiment the concentration of dimethyl sulfone may be between about 1 μM-9M. In one embodiment the concentration of dimethyl sulfone may be between about 1 μM-8.5M. In one embodiment the concentration of dimethyl sulfone may be between about 1 μM-8M. In one embodiment the concentration of dimethyl sulfone may be between about 1 μM-7.5M. In one embodiment the concentration of dimethyl sulfone may be between about 1 μM-7M. In one embodiment the concentration of dimethyl sulfone may be between about 1 μM-6.5M. In one embodiment the concentration of dimethyl sulfone may be between about 1 μM-6M. In one embodiment the concentration of dimethyl sulfone may be between about 1 μM-5.5M. In one embodiment the concentration of dimethyl sulfone may be between about 1 μM-5M. In another embodiment the concentration of dimethyl sulfone may be between about 1 μM-4.5M. In another embodiment the concentration of dimethyl sulfone may be between about 1 μM-4M. In another embodiment the concentration of dimethyl sulfone may be between about 1 μM-3.5M. In another embodiment the concentration of dimethyl sulfone may be between about 1 μM-3M. In another embodiment the concentration of dimethyl sulfone may be between about 1 μM-2.5M. In another embodiment the concentration of dimethyl sulfone may be between about 1 μM-2M. In another embodiment the concentration of dimethyl sulfone may be between about 1 μM-1.5M. In another embodiment the concentration of dimethyl sulfone may be between about 1 μM-1M. In another embodiment the concentration of dimethyl sulfone may be between about 1 μM-500 mM. In another embodiment the concentration of dimethyl sulfone may be between about 1 μM-250 mM. In another embodiment the concentration of dimethyl sulfone may be between about 1 μM-100 mM. In another embodiment the concentration of dimethyl sulfone may be between about 1 μM-50 mM. In another embodiment the concentration of dimethyl sulfone may be between about 1 μM-25 mM. In another embodiment the concentration of dimethyl sulfone may be between about 1 μM-10 mM. In another embodiment the concentration of dimethyl sulfone may be between about 1 μM-5 mM. In another embodiment the concentration of dimethyl sulfone may be between about 1 μM-1 mM. In another embodiment the concentration of dimethyl sulfone may be between about 1 μM-500 μM. In another embodiment the concentration of dimethyl sulfone may be between about 1 μM-250 μM. In another embodiment the concentration of dimethyl sulfone may be between about 1 μM-100 μM. In another embodiment the concentration of dimethyl sulfone may be between about 1 μM-50 μM. In another embodiment the concentration of dimethyl sulfone may be between about 1 μM-25 μM. In another embodiment the concentration of dimethyl sulfone may be between about 1 μM-10 μM. In another embodiment the concentration of dimethyl sulfone may be between about 1 μM-5 μM.

In one embodiment the concentration of dimethyl sulfone may be between about 1 mM-10M. In one embodiment the concentration of dimethyl sulfone may be between about 1 mM-9.5M. In one embodiment the concentration of dimethyl sulfone may be between about 1 mM-9M. In one embodiment the concentration of dimethyl sulfone may be between about 1 mM-8.5M. In one embodiment the concentration of dimethyl sulfone may be between about 1 mM-8M. In one embodiment the concentration of dimethyl sulfone may be between about 1 mM-7.5M. In one embodiment the concentration of dimethyl sulfone may be between about 1 mM-7M. In one embodiment the concentration of dimethyl sulfone may be between about 1 mM-6.5M. In one embodiment the concentration of dimethyl sulfone may be between about 1 mM-6M. In one embodiment the concentration of dimethyl sulfone may be between about 1 mM-5.5M. In another embodiment the concentration of dimethyl sulfone may be between about 1 mM-5M. In another embodiment the concentration of dimethyl sulfone may be between about 1 mM-4.5M. In another embodiment the concentration of dimethyl sulfone may be between about 1 mM-4M. In another embodiment the concentration of dimethyl sulfone may be between about 1 mM-3.5M. In another embodiment the concentration of dimethyl sulfone may be between about 1 mM-3M. In another embodiment the concentration of dimethyl sulfone may be between about 1 mM-2.5M. In another embodiment the concentration of dimethyl sulfone may be between about 1 mM-2M. In another embodiment the concentration of dimethyl sulfone may be between about 1 mM-1.5M. In another embodiment the concentration of dimethyl sulfone may be between about 1 mM-1M. In another embodiment the concentration of dimethyl sulfone may be between about 1 mM-500 mM. In another embodiment the concentration of dimethyl sulfone may be between about 1 mM-250 mM. In another embodiment the concentration of dimethyl sulfone may be between about 1 mM-100 mM. In another embodiment the concentration of dimethyl sulfone may be between about 1 mM-50 mM. In another embodiment the concentration of dimethyl sulfone may be between about 1 mM-25 mM. In another embodiment the concentration of dimethyl sulfone may be between about 1 mM-10 mM. In another embodiment the concentration of dimethyl sulfone may be between about 1 mM-5 mM.

In one embodiment the concentration of dimethyl sulfone may be between about 10 mM-10M. In one embodiment the concentration of dimethyl sulfone may be between about 10 mM-9.5M. In one embodiment the concentration of dimethyl sulfone may be between about 10 mM-9M. In one embodiment the concentration of dimethyl sulfone may be between about 10 mM-8.5M. In one embodiment the concentration of dimethyl sulfone may be between about 10 mM-8M. In one embodiment the concentration of dimethyl sulfone may be between about 10 mM-7.5M. In one embodiment the concentration of dimethyl sulfone may be between about 10 mM-7M. In one embodiment the concentration of dimethyl sulfone may be between about 10 mM-6.5M. In one embodiment the concentration of dimethyl sulfone may be between about 10 mM-6M. In one embodiment the concentration of dimethyl sulfone may be between about 10 mM-5.5M. In another embodiment the concentration of dimethyl sulfone may be between about 10 mM-5M. In another embodiment the concentration of dimethyl sulfone may be between about 10 mM-4.5M. In another embodiment the concentration of dimethyl sulfone may be between about 10 mM-4M. In another embodiment the concentration of dimethyl sulfone may be between about 10 mM-3.5M. In another embodiment the concentration of dimethyl sulfone may be between about 10 mM-3M. In another embodiment the concentration of dimethyl sulfone may be between about 10 mM-2.5M. In another embodiment the concentration of dimethyl sulfone may be between about 10 mM-2M. In another embodiment the concentration of dimethyl sulfone may be between about 10 mM-1.5M. In another embodiment the concentration of dimethyl sulfone may be between about 10 mM-1M. In another embodiment the concentration of dimethyl sulfone may be between about 10 mM-500 mM. In another embodiment the concentration of dimethyl sulfone may be between about 10 mM-250 mM. In another embodiment the concentration of dimethyl sulfone may be between about 10 mM-100 mM. In another embodiment the concentration of dimethyl sulfone may be between about 10 mM-50 mM. In another embodiment the concentration of dimethyl sulfone may be between about 10 mM-25 mM.

In one embodiment the concentration of dimethyl sulfone may be between about 25 mM-10M. In one embodiment the concentration of dimethyl sulfone may be between about 25 mM-9.5M. In one embodiment the concentration of dimethyl sulfone may be between about 25 mM-9M. In one embodiment the concentration of dimethyl sulfone may be between about 25 mM-8.5M. In one embodiment the concentration of dimethyl sulfone may be between about 25 mM-8M. In one embodiment the concentration of dimethyl sulfone may be between about 25 mM-7.5M. In one embodiment the concentration of dimethyl sulfone may be between about 25 mM-7M. In one embodiment the concentration of dimethyl sulfone may be between about 25 mM-6.5M. In one embodiment the concentration of dimethyl sulfone may be between about 25 mM-6M. In one embodiment the concentration of dimethyl sulfone may be between about 25 mM-5.5M. In another embodiment the concentration of dimethyl sulfone may be between about 25 mM-5M. In another embodiment the concentration of dimethyl sulfone may be between about 25 mM-4.5M. In another embodiment the concentration of dimethyl sulfone may be between about 25 mM-4M. In another embodiment the concentration of dimethyl sulfone may be between about 25 mM-3.5M. In another embodiment the concentration of dimethyl sulfone may be between about 25 mM-3M. In another embodiment the concentration of dimethyl sulfone may be between about 25 mM-2.5M. In another embodiment the concentration of dimethyl sulfone may be between about 25 mM-2M. In another embodiment the concentration of dimethyl sulfone may be between about 25 mM-1.5M. In another embodiment the concentration of dimethyl sulfone may be between about 25 mM-1M. In another embodiment the concentration of dimethyl sulfone may be between about 25 mM-500 mM. In another embodiment the concentration of dimethyl sulfone may be between about 25 mM-250 mM. In another embodiment the concentration of dimethyl sulfone may be between about 25 mM-100 mM. In another embodiment the concentration of dimethyl sulfone may be between about 25 mM-50 mM.

In one embodiment the concentration of dimethyl sulfone may be between about 50 mM-10M. In one embodiment the concentration of dimethyl sulfone may be between about 50 mM-9.5M. In one embodiment the concentration of dimethyl sulfone may be between about 50 mM-9M. In one embodiment the concentration of dimethyl sulfone may be between about 50 mM-8.5M. In one embodiment the concentration of dimethyl sulfone may be between about 50 mM-8M. In one embodiment the concentration of dimethyl sulfone may be between about 50 mM-7.5M. In one embodiment the concentration of dimethyl sulfone may be between about 50 mM-7M. In one embodiment the concentration of dimethyl sulfone may be between about 50 mM-6.5M. In one embodiment the concentration of dimethyl sulfone may be between about 50 mM-6M. In one embodiment the concentration of dimethyl sulfone may be between about 50 mM-5.5M. In another embodiment the concentration of dimethyl sulfone may be between about 50 mM-5M. In another embodiment the concentration of dimethyl sulfone may be between about 50 mM-4.5M. In another embodiment the concentration of dimethyl sulfone may be between about 50 mM-4M. In another embodiment the concentration of dimethyl sulfone may be between about 50 mM-3.5M. In another embodiment the concentration of dimethyl sulfone may be between about 50 mM-3M. In another embodiment the concentration of dimethyl sulfone may be between about 50 mM-2.5M. In another embodiment the concentration of dimethyl sulfone may be between about 50 mM-2M. In another embodiment the concentration of dimethyl sulfone may be between about 50 mM-1.5M. In another embodiment the concentration of dimethyl sulfone may be between about 50 mM-1M. In another embodiment the concentration of dimethyl sulfone may be between about 50 mM-500 mM. In another embodiment the concentration of dimethyl sulfone may be between about 50 mM-250 mM. In another embodiment the concentration of dimethyl sulfone may be between about 50 mM-100 mM.

In one embodiment the concentration of dimethyl sulfone may be between about 100 mM-10M. In one embodiment the concentration of dimethyl sulfone may be between about 100 mM-9.5M. In one embodiment the concentration of dimethyl sulfone may be between about 100 mM-9M. In one embodiment the concentration of dimethyl sulfone may be between about 100 mM-8.5M. In one embodiment the concentration of dimethyl sulfone may be between about 100 mM-8M. In one embodiment the concentration of dimethyl sulfone may be between about 100 mM-7.5M. In one embodiment the concentration of dimethyl sulfone may be between about 100 mM-7M. In one embodiment the concentration of dimethyl sulfone may be between about 100 mM-6.5M. In one embodiment the concentration of dimethyl sulfone may be between about 100 mM-6M. In one embodiment the concentration of dimethyl sulfone may be between about 100 mM-5.5M. In another embodiment the concentration of dimethyl sulfone may be between about 100 mM-5M. In another embodiment the concentration of dimethyl sulfone may be between about 100 mM-4.5M. In another embodiment the concentration of dimethyl sulfone may be between about 100 mM-4M. In another embodiment the concentration of dimethyl sulfone may be between about 100 mM-3.5M. In another embodiment the concentration of dimethyl sulfone may be between about 100 mM-3M. In another embodiment the concentration of dimethyl sulfone may be between about 100 mM-2.5M. In another embodiment the concentration of dimethyl sulfone may be between about 100 mM-2M. In another embodiment the concentration of dimethyl sulfone may be between about 100 mM-1.5M. In another embodiment the concentration of dimethyl sulfone may be between about 100 mM-1M. In another embodiment the concentration of dimethyl sulfone may be between about 100 mM-500 mM. In another embodiment the concentration of dimethyl sulfone may be between about 100 mM-250 mM.

In one embodiment the concentration of dimethyl sulfone may be between about 250 mM-10M. In one embodiment the concentration of dimethyl sulfone may be between about 250 mM-9.5M. In one embodiment the concentration of dimethyl sulfone may be between about 250 mM-9M. In one embodiment the concentration of dimethyl sulfone may be between about 250 mM-8.5M. In one embodiment the concentration of dimethyl sulfone may be between about 250 mM-8M. In one embodiment the concentration of dimethyl sulfone may be between about 250 mM-7.5M. In one embodiment the concentration of dimethyl sulfone may be between about 250 mM-7M. In one embodiment the concentration of dimethyl sulfone may be between about 250 mM-6.5M. In one embodiment the concentration of dimethyl sulfone may be between about 250 mM-6M. In one embodiment the concentration of dimethyl sulfone may be between about 250 mM-5.5M. In another embodiment the concentration of dimethyl sulfone may be between about 250 mM-5M. In another embodiment the concentration of dimethyl sulfone may be between about 250 mM-4.5M. In another embodiment the concentration of dimethyl sulfone may be between about 250 mM-4M. In another embodiment the concentration of dimethyl sulfone may be between about 250 mM-3.5M. In another embodiment the concentration of dimethyl sulfone may be between about 250 mM-3M. In another embodiment the concentration of dimethyl sulfone may be between about 250 mM-2.5M. In another embodiment the concentration of dimethyl sulfone may be between about 250 mM-2M. In another embodiment the concentration of dimethyl sulfone may be between about 250 mM-1.5M. In another embodiment the concentration of dimethyl sulfone may be between about 250 mM-1M. In another embodiment the concentration of dimethyl sulfone may be between about 250 mM-500 mM.

In one embodiment the concentration of dimethyl sulfone may be between about 500 mM-10M. In one embodiment the concentration of dimethyl sulfone may be between about 500 mM-9.5M. In one embodiment the concentration of dimethyl sulfone may be between about 500 mM-9M. In one embodiment the concentration of dimethyl sulfone may be between about 500 mM-8.5M. In one embodiment the concentration of dimethyl sulfone may be between about 500 mM-8M. In one embodiment the concentration of dimethyl sulfone may be between about 500 mM-7.5M. In one embodiment the concentration of dimethyl sulfone may be between about 500 mM-7M. In one embodiment the concentration of dimethyl sulfone may be between about 500 mM-6.5M. In one embodiment the concentration of dimethyl sulfone may be between about 500 mM-6M. In one embodiment the concentration of dimethyl sulfone may be between about 500 mM-5.5M. In another embodiment the concentration of dimethyl sulfone may be between about 500 mM-5M. In another embodiment the concentration of dimethyl sulfone may be between about 500 mM-4.5M. In another embodiment the concentration of dimethyl sulfone may be between about 500 mM-4M. In another embodiment the concentration of dimethyl sulfone may be between about 500 mM-3.5M. In another embodiment the concentration of dimethyl sulfone may be between about 500 mM-3M. In another embodiment the concentration of dimethyl sulfone may be between about 500 mM-2.5M. In another embodiment the concentration of dimethyl sulfone may be between about 500 mM-2M. In another embodiment the concentration of dimethyl sulfone may be between about 500 mM-1.5M. In another embodiment the concentration of dimethyl sulfone may be between about 500 mM-1M.

In one embodiment the concentration of dimethyl sulfone may be between about 1M-10M. In one embodiment the concentration of dimethyl sulfone may be between about 1M-9.5M. In one embodiment the concentration of dimethyl sulfone may be between about 1M-9M. In one embodiment the concentration of dimethyl sulfone may be between about 1M-8.5M. In one embodiment the concentration of dimethyl sulfone may be between about 1M-8M. In one embodiment the concentration of dimethyl sulfone may be between about 1M-7.5M. In one embodiment the concentration of dimethyl sulfone may be between about 1M-7M. In one embodiment the concentration of dimethyl sulfone may be between about 1M-6.5M. In one embodiment the concentration of dimethyl sulfone may be between about 1M-6M. In one embodiment the concentration of dimethyl sulfone may be between about 1M-5.5M. In another embodiment the concentration of dimethyl sulfone may be between about 1M-5M. In another embodiment the concentration of dimethyl sulfone may be between about 1M-4.5M. In another embodiment the concentration of dimethyl sulfone may be between about 1M-4M. In another embodiment the concentration of dimethyl sulfone may be between about 1M-3.5M. In another embodiment the concentration of dimethyl sulfone may be between about 1M-3M. In another embodiment the concentration of dimethyl sulfone may be between about 1M-2.5M. In another embodiment the concentration of dimethyl sulfone may be between about 1M-2M.

Embodiments of compositions comprising one or more RNA stabilizing substance, such as an aprotic substance of which dimethyl sulfone is a non-limiting example, may have different molar concentrations. For conciseness, the following list of molar concentrations, with dimethyl sulfone as a non-limiting example aprotic substance, is herein referred to as the stabilizing substance molar concentration list, and herein also referred to as the RNA stabilizing substance molar concentration list.

In one embodiment the concentration of dimethyl sulfone may be at least 1 nM or more. In one embodiment the concentration of dimethyl sulfone may be at least 10 nM or more. In one embodiment the concentration of dimethyl sulfone may be at least 100 nM or more. In one embodiment the concentration of dimethyl sulfone may be at least 1 μM or more. In one embodiment the concentration of dimethyl sulfone may be at least 10 μM or more. In one embodiment the concentration of dimethyl sulfone may be at least 100 μM or more. In one embodiment the concentration of dimethyl sulfone may be at least 500 μM or more. In one embodiment the concentration of dimethyl sulfone may be at least 1 mM or more. In one embodiment the concentration of dimethyl sulfone may be at least 10 mM or more. In one embodiment the concentration of dimethyl sulfone may be at least 20 mM or more. In one embodiment the concentration of dimethyl sulfone may be at least 50 mM or more. In one embodiment the concentration of dimethyl sulfone may be at least 100 mM or more. In one embodiment the concentration of dimethyl sulfone may be at least 150 mM or more. In one embodiment the concentration of dimethyl sulfone may be at least 200 mM or more. In one embodiment the concentration of dimethyl sulfone may be at least 250 mM or more. In one embodiment the concentration of dimethyl sulfone may be at least 300 mM or more. In one embodiment the concentration of dimethyl sulfone may be at least 350 mM or more. In one embodiment the concentration of dimethyl sulfone may be at least 400 mM or more. In one embodiment the concentration of dimethyl sulfone may be at least 500 mM or more. In one embodiment the concentration of dimethyl sulfone may be at least 600 mM or more. In one embodiment the concentration of dimethyl sulfone may be at least 700 mM or more. In one embodiment the concentration of dimethyl sulfone may be at least 800 mM or more. In one embodiment the concentration of dimethyl sulfone may be at least 900 mM or more. In one embodiment the concentration of dimethyl sulfone may be at least 1M or more. In one embodiment the concentration of dimethyl sulfone may be at least 1.5M or more. In one embodiment the concentration of dimethyl sulfone may be at least 2M or more. In one embodiment the concentration of dimethyl sulfone may be at least 3M or more. In one embodiment the concentration of dimethyl sulfone may be at least 4M or more. In one embodiment the concentration of dimethyl sulfone may be at least 5M or more. In one embodiment the concentration of dimethyl sulfone may be at least 6M or more.

A non-limiting example of an aprotic substance that may be used is diethyl sulfoxide (DESO), wherein diethyl sulfoxide may be substituted for or used in combination with one or more aprotic substance as described herein. One embodiment of the present invention may include combinations of one or more RNA stabilizing substances comprising diethyl sulfoxide and another embodiment may also comprise at least one RNA substance. Other embodiments may include combinations of one or more RNA stabilizing substances comprising diethyl sulfoxide wherein the concentration of diethyl sulfoxide may be one or more concentration in the stabilizing substance weight percent concentration list or in the stabilizing substance molar concentration range list or in the stabilizing substance molar concentration list.

A non-limiting example of an aprotic substance that may be used is dimethyl sulfoxide (DMSO), wherein dimethyl sulfoxide may be substituted for or used in combination with one or more aprotic substance as described herein. One embodiment of the present invention may include combinations of one or more RNA stabilizing substances comprising dimethyl sulfoxide and another embodiment may also comprise at least one RNA substance. Other embodiments may include combinations of one or more RNA stabilizing substances comprising dimethyl sulfoxide wherein the concentration of dimethyl sulfoxide may be one or more concentration in the stabilizing substance weight percent concentration list or in the stabilizing substance molar concentration range list or in the stabilizing substance molar concentration list.

A non-limiting example of an aprotic substance that may be used is dimethyl sulfone (also known as DMSO₂, methylsulfonylmethane, MSM, or methyl sulfone), wherein dimethyl sulfone may be substituted for or used in combination with one or more aprotic substance as described herein. One embodiment of the present invention may include combinations of one or more RNA stabilizing substances comprising dimethyl sulfone and another embodiment may also comprise at least one RNA substance. Other embodiments may include combinations of one or more RNA stabilizing substances comprising dimethyl sulfone wherein the concentration of dimethyl sulfone may be one or more concentration in the stabilizing substance weight percent concentration list or in the stabilizing substance molar concentration range list or in the stabilizing substance molar concentration list.

A non-limiting example of an aprotic substance that may be used is N-Methyl-2-pyrrolidone (also known as, 1-Methyl-2-pyrrolidinone, or NMP), wherein NMP may be substituted for or used in combination with one or more aprotic substance as described herein. One embodiment of the present invention may include combinations of one or more RNA stabilizing substances comprising NMP and another embodiment may also comprise at least one RNA substance. Other embodiments may include combinations of one or more RNA stabilizing substances comprising NMP wherein the concentration of NMP may be one or more concentration in the stabilizing substance weight percent concentration list or in the stabilizing substance molar concentration range list or in the stabilizing substance molar concentration list.

In one embodiment one or more aprotic substance may comprise one or more ester. In one embodiment one or more aprotic substance may comprise one or more carboxylate ester. In one embodiment one or more aprotic substance may comprise one or more acyclic carboxylate ester. In one embodiment one or more aprotic substance may comprise one or more cyclic carboxylate ester.

In one embodiment one more aprotic substance may comprise a carboxylate ester comprising the formula ZiO(C═O)Z₂, wherein Z₁ and Z₂ are independently selected Z groups comprising at least one carbon atom, and Z₁ or Z₂ may be the same or different.

In a non-limiting example of an aprotic carboxylate ester, Z₁ may be a butyl group comprising four carbon atoms and Z₂ may be a methyl group comprising one carbon, wherein an aprotic carboxylate ester may be butyl acetate.

In one embodiment one or more aprotic substance may comprise a carbonate ester. In one embodiment one or more aprotic substance may comprise one or more acyclic carbonate ester. In one embodiment one or more aprotic substance may comprise one or more cyclic carbonate ester.

In one embodiment one more aprotic substance may comprise a carbonate ester comprising the formula Z₁O(C═O)OZ₂, wherein Z₁ and Z₂ are independently selected Z groups comprising at least one carbon atom, and Z₁ or Z₂ may be the same or different.

In a non-limiting example of an aprotic carbonate ester, Z₁ may be an ethyl group comprising two carbon atoms and Z₂ may be an ethyl group comprising two carbons, wherein an aprotic carbonate ester may be diethyl carbonate.

In one embodiment a Z group may be comprised of at least one carbon atom, wherein Z may be comprised of C1-16 alkyl, alkenyl, alkynyl, aryl, or aralkyl group, each of which may contain up to 8 heteroatoms. In another embodiment a Z group may be comprised of C1-12 alkyl, alkenyl, alkynyl, aryl, or aralkyl group, each of which may contain up to 6 heteroatoms. In another embodiment a Z group may be comprised of C1-8 alkyl, alkenyl, alkynyl, aryl, or aralkyl group, each of which may contain up to 4 heteroatoms. In another embodiment a Z group may be comprised of C1-6 alkyl, alkenyl, alkynyl, aryl, or aralkyl group, each of which may contain up to 3 heteroatoms.

In another embodiment one or more Z groups may be aprotic. In another embodiment one or more Z groups may form a ring structure. In another embodiment one or more Z groups may form at least part of a ring structure. In another embodiment one or more Z groups may be at least part of a ring structure. In another embodiment one or more Z groups may comprise at least part of a ring structure.

In one embodiment a Z group may be covalently bonded to another Z group. In one embodiment one or more Z group may form a heterocyclic ring structure. In one embodiment one or more Z group may form at least part of a heterocyclic ring structure. In one embodiment one or more Z group may be at least part of a heterocyclic ring structure. In one embodiment one or more Z group may comprise at least part of a heterocyclic ring structure.

A non-limiting example of an aprotic substance comprised of a carboxylate ester that may be used is butyl acetate (also known as butyl ethanoate), wherein butyl acetate may be substituted for or used in combination with one or more aprotic substance as described herein. One embodiment of the present invention may include combinations of one or more RNA stabilizing substances comprising butyl acetate and another embodiment may also comprise at least one RNA substance. Other embodiments may include combinations of one or more RNA stabilizing substances comprising butyl acetate wherein the concentration of butyl acetate may be one or more concentration in the stabilizing substance weight percent concentration list or in the stabilizing substance molar concentration range list or in the stabilizing substance molar concentration list.

A non-limiting example of an aprotic substance comprised of a carbonate ester that may be used is diethyl carbonate (also known as DEC), wherein diethyl carbonate may be substituted for or used in combination with one or more aprotic substance as described herein. One embodiment of the present invention may include combinations of one or more RNA stabilizing substances comprising diethyl carbonate and another embodiment may also comprise at least one RNA substance. Other embodiments may include combinations of one or more RNA stabilizing substances comprising diethyl carbonate wherein the concentration of diethyl carbonate may be one or more concentration in the stabilizing substance weight percent concentration list or in the stabilizing substance molar concentration range list or in the stabilizing substance molar concentration list.

A non-limiting example of an aprotic substance comprised of a cyclic carbonate ester that may be used is propylene carbonate (also known as 4-methyl-1,3-dioxolan-2-one or 1,2-propanediol cyclic carbonate), wherein propylene carbonate may be substituted for or used in combination with one or more aprotic substance as described herein. One embodiment of the present invention may include combinations of one or more RNA stabilizing substances comprising propylene carbonate and another embodiment may also comprise at least one RNA substance. Other embodiments may include combinations of one or more RNA stabilizing substances comprising propylene carbonate wherein the concentration of propylene carbonate may be one or more concentration in the stabilizing substance weight percent concentration list or in the stabilizing substance molar concentration range list or in the stabilizing substance molar concentration list.

In one embodiment one or more aprotic substance may comprise glycerine acetate.

A non-limiting example of an aprotic substance comprising glycerin acetate that may be used is triacetin (also known as 1,2,3-triacetylglycerol or glycerin triacetate), wherein triacetin may be substituted for or used in combination with one or more aprotic substance as described herein. One embodiment of the present invention may include combinations of one or more RNA stabilizing substances comprising triacetin and another embodiment may also comprise at least one RNA substance. Other embodiments may include combinations of one or more RNA stabilizing substances comprising triacetin wherein the concentration of triacetin may be one or more concentration in the stabilizing substance weight percent concentration list or in the stabilizing substance molar concentration range list or in the stabilizing substance molar concentration list.

In one embodiment an aprotic substance may comprise one or more choline-based ester.

A non-limiting example of an aprotic substance comprised of one or more choline-based ester that may be used is acetylcholine, wherein acetylcholine may be substituted for or used in combination with one or more aprotic substance as described herein. One embodiment of the present invention may include combinations of one or more RNA stabilizing substances comprising acetylcholine and another embodiment may also comprise at least one RNA substance. Other embodiments may include combinations of one or more RNA stabilizing substances comprising acetylcholine wherein the concentration of acetylcholine may be one or more concentration in the stabilizing substance weight percent concentration list or in the stabilizing substance molar concentration range list or in the stabilizing substance molar concentration list.

A non-limiting example of an aprotic substance comprised of one or more choline-based ester that may be used is butyrylcholine, wherein butyrylcholine may be substituted for or used in combination with one or more aprotic substance as described herein. One embodiment of the present invention may include combinations of one or more RNA stabilizing substances comprising butyrylcholine and another embodiment may also comprise at least one RNA substance. Other embodiments may include combinations of one or more RNA stabilizing substances comprising butyrylcholine wherein the concentration of butyrylcholine may be one or more concentration in the stabilizing substance weight percent concentration list or in the stabilizing substance molar concentration range list or in the stabilizing substance molar concentration list.

In one embodiment an aprotic substance may comprise a diester comprising the formula ZiO(C═O)Cₙ(C═O)OZ₂, wherein Z₁ and Z₂ are independently selected Z groups comprising at least one carbon atom, and Z₁ or Z₂ may be the same or different.

In one embodiment n may be an integer between 0-10, wherein n may be 0, 1, 2, 3, 4, 5, 6, 7, 8, 9, or 10. In one embodiment n may be an integer between 0-5, wherein n may be 0, 1, 2, 3, 4, or 5.

In a non-limiting example of an aprotic diester, Z₁ may be a methyl group comprising one carbon atom, n=1, and Z₂ may be a methyl group comprising one carbon, wherein an aprotic diester may be dimethyl malonate.

In another non-limiting example of an aprotic diester, Z₁ may be a methyl group comprising one carbon atom, n=0, and Z₂ may be a methyl group comprising one carbon, wherein an aprotic diester may be dimethyl oxalate.

A non-limiting example of an aprotic substance comprising a diester that may be used is dimethyl malonate (also known as dimethyl propanedioate or malonic acid dimethyl ester), wherein dimethyl malonate may be substituted for or used in combination with one or more aprotic substance as described herein. One embodiment of the present invention may include combinations of one or more RNA stabilizing substances comprising dimethyl malonate and another embodiment may also comprise at least one RNA substance. Other embodiments may include combinations of one or more RNA stabilizing substances comprising dimethyl malonate wherein the concentration of dimethyl malonate may be one or more concentration in the stabilizing substance weight percent concentration list or in the stabilizing substance molar concentration range list or in the stabilizing substance molar concentration list.

A non-limiting example of an aprotic substance comprising a diester that may be used is diethyl malonate (also known as diethyl propanedioate or DEM), wherein diethyl malonate may be substituted for or used in combination with one or more aprotic substance as described herein. One embodiment of the present invention may include combinations of one or more RNA stabilizing substances comprising diethyl malonate and another embodiment may also comprise at least one RNA substance. Other embodiments may include combinations of one or more RNA stabilizing substances comprising diethyl malonate wherein the concentration of diethyl malonate may be one or more concentration in the stabilizing substance weight percent concentration list or in the stabilizing substance molar concentration range list or in the stabilizing substance molar concentration list.

A non-limiting example of an aprotic substance comprising a diester that may be used is dimethyl oxalate, wherein dimethyl oxalate may be substituted for or used in combination with one or more aprotic substance as described herein. One embodiment of the present invention may include combinations of one or more RNA stabilizing substances comprising dimethyl oxalate and another embodiment may also comprise at least one RNA substance. Other embodiments may include combinations of one or more RNA stabilizing substances comprising dimethyl oxalate wherein the concentration of dimethyl oxalate may be one or more concentration in the stabilizing substance weight percent concentration list or in the stabilizing substance molar concentration range list or in the stabilizing substance molar concentration list.

For conciseness, the following list of aprotic substances, is herein referred to as the aprotic substance list wherein one or more of the following substances may be substituted for or used in combination with one or more aprotic substance as described herein.

In other embodiments, other aprotic substances that may be substituted for or used in combination with one or more aprotic substance as described herein may comprise one or more of the following, including but not limited to: DMSO, diethyl sulfoxide, dimethyl sulfone, triacetin, N-methyl-2-pyrrolidone, acetylcholine, butyrylcholine, methacholine, diethyl carbonate, propylene carbonate, ethyl acetate, methyl benzoate, ethylene carbonate, dimethyl carbonate, ethylmethyl carbonate, ethyl pentanoate, butyl acetate, isobutyl acetate, isobutyl valerate, methyl butyrate, phenylethyl acetate, propyl butyrate, bornyl acetate, diethyl oxalate, dibutyl oxalate, tetraethyl pentane-1,3,3,5-tetracarboxylate, tetramethyl butane-1,1,4,4-tetracarboxylate, diethyl succinosuccinate, diethyl succinate, ethyl acetoacetate, ethyl benzoate, ethyl benzoylacetate, benzyl acetate, methyl acetate, sulfolane, dimethyl malonate, diethyl malonate, dimethyl oxalate, diethyl oxalate, diphenyl oxalate, methyl dihydrojasmonate, methyl jasmonate, or acetone, or mixtures, or combinations thereof.

Embodiments of the present invention may include combinations comprising one or more of the substances from the aprotic substance list substituted for or used in combination with one or more aprotic substance or one or more RNA stabilizing substance, such as a betaine containing substance as a non-limiting example, and other embodiments may also comprise at least one RNA substance.

Other embodiments may include combinations of substances comprising one or more aprotic substance selected from the aprotic substance list wherein the concentration of one or more substances may be one or more concentration in the stabilizing substance weight percent concentration list or in the stabilizing substance molar concentration range list or in the stabilizing substance molar concentration list.

In one embodiment, an aprotic substance may have a molecular weight between about 25-1,000,000 daltons. In one embodiment, an aprotic substance may have a molecular weight between about 25-500,000 daltons. In one embodiment, an aprotic substance may have a molecular weight between about 25-200,000 daltons. In one embodiment, an aprotic substance may have a molecular weight between about 25-100,000 daltons. In one embodiment, an aprotic substance may have a molecular weight between about 25-50,000 daltons. In one embodiment, an aprotic substance may have a molecular weight between about 25-25,000 daltons. In one embodiment, an aprotic substance may have a molecular weight between about 25-10,000 daltons. In one embodiment, an aprotic substance may have a molecular weight between about 25-5,000 daltons. In one embodiment, an aprotic substance may have a molecular weight between about 25-2,000 daltons. In one embodiment, an aprotic substance may have a molecular weight between about 25-1,000 daltons. In one embodiment, an aprotic substance may have a molecular weight between about 25-900 daltons. In one embodiment, an aprotic substance may have a molecular weight between about 25-800 daltons. In one embodiment, an aprotic substance may have a molecular weight between about 25-700 daltons. In one embodiment, an aprotic substance may have a molecular weight between about 25-600 daltons. In one embodiment, an aprotic substance may have a molecular weight between about 25-500 daltons. In one embodiment, an aprotic substance may have a molecular weight between about 25-400 daltons. In one embodiment, an aprotic substance may have a molecular weight between about 25-300 daltons. In one embodiment, an aprotic substance may have a molecular weight between about 25-250 daltons.

In one embodiment, an aprotic substance may have a molecular weight between about 50-1,000,000 daltons. In one embodiment, an aprotic substance may have a molecular weight between about 50-500,000 daltons. In one embodiment, an aprotic substance may have a molecular weight between about 50-200,000 daltons. In one embodiment, an aprotic substance may have a molecular weight between about 50-100,000 daltons. In one embodiment, an aprotic substance may have a molecular weight between about 50-50,000 daltons. In one embodiment, an aprotic substance may have a molecular weight between about 50-25,000 daltons. In one embodiment, an aprotic substance may have a molecular weight between about 50-10,000 daltons. In one embodiment, an aprotic substance may have a molecular weight between about 50-5,000 daltons. In one embodiment, an aprotic substance may have a molecular weight between about 50-2,000 daltons. In one embodiment, an aprotic substance may have a molecular weight between about 50-1,000 daltons. In one embodiment, an aprotic substance may have a molecular weight between about 50-900 daltons. In one embodiment, an aprotic substance may have a molecular weight between about 50-800 daltons. In one embodiment, an aprotic substance may have a molecular weight between about 50-700 daltons. In one embodiment, an aprotic substance may have a molecular weight between about 50-600 daltons. In one embodiment, an aprotic substance may have a molecular weight between about 50-500 daltons. In one embodiment, an aprotic substance may have a molecular weight between about 50-400 daltons. In one embodiment, an aprotic substance may have a molecular weight between about 50-300 daltons. In one embodiment, an aprotic substance may have a molecular weight between about 50-250 daltons.

One embodiment of the present invention is the method whereby one or more aprotic substance may be combined, such as by mixing, with at least one or more RNA substance to produce a mixture comprising at least one or more aprotic substance and at least one or more RNA substance. As a non-limiting example one or more RNA substance may be mixed with one or more aprotic substance.

Another embodiment of the present invention is the method whereby one or more aprotic substance may be combined, such as by mixing, with at least one or more RNA substance to produce a mixture comprising at least one or more RNA substance and at least one or more aprotic substance at one or more of the RNA stabilizing substance concentrations within the stabilizing substance molar concentration range list or in the stabilizing substance molar concentration list or the stabilizing substance weight percent concentration list as described herein. These same methods may be used to combine one or more other substances, including, but not limited to one or more cellular uptake agents or one or more additional RNA stabilizing substances, with one or more RNA substances and one or more aprotic substances to produce a mixture comprising one or more RNA substance, one or more aprotic substance, and one or more cellular uptake agent or one or more additional RNA stabilizing substance.

One embodiment of the present invention is the method whereby one or more aprotic substance may be combined, such as by mixing, with at least one or more RNA substance to produce a composition comprising at least one or more RNA substance and at least one or more aprotic substance. Another embodiment of the present invention is the method whereby one or more aprotic substance may be combined, such as by mixing, with one or more RNA substance to produce a composition comprising at least one or more RNA substance and at least one or more aprotic substance at one or more of the RNA stabilizing substance concentrations within the stabilizing substance molar concentration range list or in the stabilizing substance molar concentration list or stabilizing substance weight percent concentration list as described herein. These same methods may be used to combine one or more other substances, including, but not limited to one or more cellular uptake agents or one or more additional RNA stabilizing substance, with one or more RNA substance and one or more aprotic substance to produce a composition comprising one or more RNA substance, one or more aprotic substance, and one or more cellular uptake agent or one or more additional RNA stabilizing substance.

Embodiments of the present invention may include compositions that comprise at least one RNA stabilizing substance, at least one RNA substance, and water. These embodiments comprising water may include one or more composition as described herein that may also comprise water.

In one embodiment an RNA stabilizing substance may comprise one or more sulfur atom. In one embodiment an RNA stabilizing substance may comprise one or more nitrogen atom. In one embodiment an aprotic substance may comprise one or more sulfur atom. In one embodiment an aprotic substance may comprise one or more nitrogen atom.

In one embodiment an RNA stabilizing substance may comprise a polar aprotic substance. In one embodiment an aprotic substance may comprise a polar aprotic substance. In one embodiment a polar aprotic substance may comprise at least one oxygen atom. In one embodiment a polar aprotic substance may comprise at least one nitrogen atom. In one embodiment a polar aprotic substance may comprise at least one sulfur atom.

Embodiments of the present invention may include compositions that comprise at least one RNA stabilizing substance, at least one RNA substance, and one or more buffering agent. These embodiments comprising one or more buffering agent may include one or more composition as described herein that may also comprise one or more buffering agent. In one embodiment one or more composition comprising at least one or more RNA substance and one or more RNA stabilizing substance may comprise a buffering agent at about physiologic pH.

In one embodiment a buffering agent may be present in concentrations from about 1 mM to 5M, such as about 1 mM, 5 mM, 10 mM, 20 mM, 30 mM, 40 mM, 50 mM, 100 mM, 200 mM, 300 mM, 400 mM, 500 mM, 750 mM, 1M, 1.5M, 2M, 2.5M, 3M, 3.5M, 4M, 4.5M, or 5M. In one embodiment a buffering agent may be present in concentrations between about 1 mM-1M, such as about 1 mM, 5 mM, 10 mM, 20 mM, 30 mM, 40 mM, 50 mM, 100 mM, 200 mM, 300 mM, 400 mM, 500 mM, 750 mM, or 1M. In another embodiment a buffering agent may be present in concentrations between about 10 mM-1M, such as about 10 mM, 20 mM, 30 mM, 40 mM, 50 mM, 100 mM, 200 mM, 300 mM, 400 mM, 500 mM, 750 mM, or 1M. In another embodiment a buffering agent may be present in concentrations between about 10 mM-500 mM, such as about 10 mM, 20 mM, 30 mM, 40 mM, 50 mM, 100 mM, 200 mM, 300 mM, 400 mM, or 500 mM. In another embodiment a buffering agent may be present in concentrations between about 10 mM-200 mM, such as about 10 mM, 20 mM, 30 mM, 40 mM, 50 mM, 100 mM, or 200 mM. In another embodiment a buffering agent may be present in concentrations between about 10 mM-100 mM, such as about 10 mM, 20 mM, 30 mM, 40 mM, 50 mM, or 100 mM.

Embodiments of the present invention may include compositions that comprise at least one RNA stabilizing substance, at least one RNA substance, and one or more inorganic salt. These embodiments comprising one or more inorganic salt may include one or more composition as described herein that may also comprise one or more inorganic salt.

In one embodiment an inorganic salt may be present in concentrations between about 1 mM to 5M, such as about 1 mM, 5 mM, 10 mM, 20 mM, 30 mM, 40 mM, 50 mM, 100 mM, 200 mM, 300 mM, 400 mM, 500 mM, 750 mM, 1M, 1.5M, 2M, 2.5M, 3M, 3.5M, 4M, 4.5M, or 5M. In one embodiment an inorganic salt may be present in concentrations between about 1 mM to 2M, such as about 1 mM, 5 mM, 10 mM, 20 mM, 30 mM, 40 mM, 50 mM, 100 mM, 200 mM, 300 mM, 400 mM, 500 mM, 750 mM, 1M, 1.5M, or 2M. In another embodiment an inorganic salt may be present in concentrations between about 10 mM-1M, such as about 10 mM, 20 mM, 30 mM, 40 mM, 50 mM, 100 mM, 200 mM, 300 mM, 400 mM, 500 mM, 750 mM, or 1M. In another embodiment an inorganic salt may be present in concentrations between about 10 mM-500 mM, such as about 10 mM, 20 mM, 30 mM, 40 mM, 50 mM, 100 mM, 200 mM, 300 mM, 400 mM, or 500 mM. In another embodiment an inorganic salt may be present in concentrations between about 10 mM-200 mM, such as about 10 mM, 20 mM, 30 mM, 40 mM, 50 mM, 100 mM, or 200 mM. In another embodiment an inorganic salt may be present in concentrations between about 1 mM-100 mM, such as about 1 mM, 5 mM, 10 mM, 20 mM, 50 mM, 75 mM, or 100 mM. In another embodiment an inorganic salt may be present in concentrations between about 1 mM-50 mM, such as about 1 mM, 5 mM, 10 mM, 15 mM, 20 mM, 25 mM, 30 mM, 40 mM or 50 mM.

Embodiments of the present invention may include compositions that comprise at least one RNA stabilizing substance, at least one RNA substance, and one or more chelating agent. These embodiments comprising one or more chelating agent may include one or more composition as described herein that may also comprise one or more chelating agent.

In one embodiment a chelating agent may be present in concentrations between about 0.1 mM to 5M, such as about 0.1 mM, 0.5 mM, 1 mM, 5 mM, 10 mM, 20 mM, 30 mM, 40 mM, 50 mM, 100 mM, 200 mM, 300 mM, 400 mM, 500 mM, 750 mM, 1M, 1.5M, 2M, 2.5M, 3M, 3.5M, 4M, 4.5M, or 5M. In one embodiment a chelating agent may be present in concentrations between about 0.1 mM to 1M, such as about 0.1 mM, 0.5 mM, 1 mM, 5 mM, 10 mM, 20 mM, 30 mM, 40 mM, 50 mM, 100 mM, 200 mM, 300 mM, 400 mM, 500 mM, 750 mM, or 1M. In another embodiment a chelating agent may be present in concentrations between about 0.1 mM-500 mM, such as about 0.1 mM, 0.5 mM, 1 mM, 5 mM, 10 mM, 20 mM, 30 mM, 40 mM, 50 mM, 100 mM, 200 mM, 300 mM, 400 mM, or 500 mM. In another embodiment a chelating agent may be present between about 0.1 mM-100 mM, such as about 0.1 mM, 0.5 mM, 1 mM, 5 mM, 10 mM, 20 mM, 30 mM, 40 mM, 50 mM, or 100 mM. In another embodiment a chelating agent may be present between about 0.1 mM-10 mM, such as about 0.1 mM, 0.5 mM, 1 mM, 5 mM, or 10 mM.

In one embodiment a composition comprising at least one RNA substance and at least one RNA stabilizing substance may be a liquid. As non-limiting examples, may be a solution, fluid, syrup, emulsion, or suspension and may also include liquid or solid carriers. As non-limiting examples the liquid viscosity may be in the range between about 0.1 centipoise-100,000,000 centipoise at about 20-25° C. As non-limiting examples the liquid viscosity may be in the range between about 0.1 centipoise-1,000,000 centipoise at about 20-25° C. As non-limiting examples the liquid viscosity may be in the range between about 0.1 centipoise-100,000 centipoise at about 20-25° C. As non-limiting examples the liquid viscosity may be in the range between about 0.1 centipoise-10,000 centipoise at about 20-25° C. As non-limiting examples the liquid viscosity may be in the range between about 0.1 centipoise-1,000 centipoise at about 20-25° C.

In one embodiment a composition comprising at least one RNA substance and at least one RNA stabilizing substance may be a solid. As non-limiting examples, a composition comprising at least one RNA substance and at least one RNA stabilizing substance may be a pellet, powder, or tablet, and may also include solid carriers.

In one embodiment a composition comprising at least one RNA substance and at least one RNA stabilizing substance may be a gel. As non-limiting examples, a composition comprising at least one RNA substance and at least one RNA stabilizing substance may be a hydrated solid or porous solid filled with or retaining water or other liquid or solution, and may also include solid or liquid carriers.

In one embodiment a combination comprising at least one RNA substance and at least one RNA stabilizing substance may be a vapor or aerosol. As non-limiting examples, may be a gas, vapor, or aerosol, or suspension of particles or droplets suspended in one or more gases (such as, but not limited to, air, nitrogen, oxygen, carbon dioxide, or anesthetic gas) and may also include liquid or solid carriers.

In one embodiment, an RNA stabilizing substance may have a molecular weight between about 25-1,000,000,000 daltons. In one embodiment, an RNA stabilizing substance may have a molecular weight between about 25-100,000,000 daltons. In one embodiment, an RNA stabilizing substance may have a molecular weight between about 25-10,000,000 daltons. In one embodiment, an RNA stabilizing substance may have a molecular weight between about 25-1,000,000 daltons. In one embodiment, an RNA stabilizing substance may have a molecular weight between about 25-500,000 daltons. In one embodiment, an RNA stabilizing substance may have a molecular weight between about 25-200,000 daltons. In one embodiment, an RNA stabilizing substance may have a molecular weight between about 25-100,000 daltons. In one embodiment, an RNA stabilizing substance may have a molecular weight between about 25-50,000 daltons. In one embodiment, an RNA stabilizing substance may have a molecular weight between about 25-25,000 daltons. In one embodiment, an RNA stabilizing substance may have a molecular weight between about 25-10,000 daltons. In one embodiment, an RNA stabilizing substance may have a molecular weight between about 25-5,000 daltons. In one embodiment, an RNA stabilizing substance may have a molecular weight between about 25-2,000 daltons. In one embodiment, an RNA stabilizing substance may have a molecular weight between about 25-1,000 daltons. In one embodiment, an RNA stabilizing substance may have a molecular weight between about 25-900 daltons. In one embodiment, an RNA stabilizing substance may have a molecular weight between about 25-800 daltons. In one embodiment, an RNA stabilizing substance may have a molecular weight between about 25-700 daltons. In one embodiment, an RNA stabilizing substance may have a molecular weight between about 25-600 daltons. In one embodiment, an RNA stabilizing substance may have a molecular weight between about 25-500 daltons. In one embodiment, an RNA stabilizing substance may have a molecular weight between about 25-400 daltons. In one embodiment, an RNA stabilizing substance may have a molecular weight between about 25-300 daltons. In one embodiment, an RNA stabilizing substance may have a molecular weight between about 25-250 daltons.

In one embodiment, an RNA stabilizing substance may have a molecular weight between about 50-1,000,000,000 daltons. In one embodiment, an RNA stabilizing substance may have a molecular weight between about 50-100,000,000 daltons. In one embodiment, an RNA stabilizing substance may have a molecular weight between about 50-10,000,000 daltons. In one embodiment, an RNA stabilizing substance may have a molecular weight between about 50-1,000,000 daltons. In one embodiment, an RNA stabilizing substance may have a molecular weight between about 50-500,000 daltons. In one embodiment, an RNA stabilizing substance may have a molecular weight between about 50-200,000 daltons. In one embodiment, an RNA stabilizing substance may have a molecular weight between about 50-100,000 daltons. In one embodiment, an RNA stabilizing substance may have a molecular weight between about 50-50,000 daltons. In one embodiment, an RNA stabilizing substance may have a molecular weight between about 50-25,000 daltons. In one embodiment, an RNA stabilizing substance may have a molecular weight between about 50-10,000 daltons. In one embodiment, an RNA stabilizing substance may have a molecular weight between about 50-5,000 daltons. In one embodiment, an RNA stabilizing substance may have a molecular weight between about 50-2,000 daltons. In one embodiment, an RNA stabilizing substance may have a molecular weight between about 50-1,000 daltons. In one embodiment, an RNA stabilizing substance may have a molecular weight between about 50-900 daltons. In one embodiment, an RNA stabilizing substance may have a molecular weight between about 50-800 daltons. In one embodiment, an RNA stabilizing substance may have a molecular weight between about 50-700 daltons. In one embodiment, an RNA stabilizing substance may have a molecular weight between about 50-600 daltons. In one embodiment, an RNA stabilizing substance may have a molecular weight between about 50-500 daltons. In one embodiment, an RNA stabilizing substance may have a molecular weight between about 50-400 daltons. In one embodiment, an RNA stabilizing substance may have a molecular weight between about 50-300 daltons. In one embodiment, an RNA stabilizing substance may have a molecular weight between about 50-250 daltons.

In one embodiment, an RNA stabilizing substance may have a molecular greater than about 50 daltons. In one embodiment, an RNA stabilizing substance may have a molecular greater than about 75 daltons. In one embodiment, an RNA stabilizing substance may have a molecular greater than about 100 daltons. In one embodiment, an RNA stabilizing substance may have a molecular greater than about 125 daltons. In one embodiment, an RNA stabilizing substance may have a molecular greater than about 150 daltons. In one embodiment, an RNA stabilizing substance may have a molecular greater than about 175 daltons. In one embodiment, an RNA stabilizing substance may have a molecular greater than about 200 daltons. In one embodiment, an RNA stabilizing substance may have a molecular greater than about 300 daltons. In one embodiment, an RNA stabilizing substance may have a molecular greater than about 350 daltons. In one embodiment, an RNA stabilizing substance may have a molecular greater than about 400 daltons. In one embodiment, an RNA stabilizing substance may have a molecular greater than about 450 daltons. In one embodiment, an RNA stabilizing substance may have a molecular greater than about 500 daltons. In one embodiment, an RNA stabilizing substance may have a molecular greater than about 600 daltons. In one embodiment, an RNA stabilizing substance may have a molecular greater than about 700 daltons. In one embodiment, an RNA stabilizing substance may have a molecular greater than about 800 daltons. In one embodiment, an RNA stabilizing substance may have a molecular greater than about 900 daltons. In one embodiment, an RNA stabilizing substance may have a molecular greater than about 1,000 daltons. In one embodiment, an RNA stabilizing substance may have a molecular greater than about 2,000 daltons. In one embodiment, an RNA stabilizing substance may have a molecular greater than about 5,000 daltons. In one embodiment, an RNA stabilizing substance may have a molecular greater than about 10,000 daltons. In one embodiment, an RNA stabilizing substance may have a molecular greater than about 20,000 daltons. In one embodiment, an RNA stabilizing substance may have a molecular greater than about 50,000 daltons. In one embodiment, an RNA stabilizing substance may have a molecular greater than about 100,000 daltons.

In one embodiment one or more composition comprising one or more RNA substance and one or more aprotic substance may comprise one or more water concentrations in the water concentration list or in the water concentration range list. In one embodiment one or more composition comprising one or more RNA substance, one or more aprotic substance, and one or more additional substance, such as one or more cellular uptake agent or one or more additional RNA stabilizing substance as non-limiting examples, may comprise one or more water concentrations in the water concentration list or in the water concentration range list. In one embodiment one or more composition comprising one or more RNA substance and one or more aprotic substance may have a dielectric constant of one or more dielectric constant in the dielectric constant list or in the dielectric constant range list. In one embodiment one or more composition comprising one or more RNA substance, one or more aprotic substance, and one or more additional substance, such as one or more cellular uptake agent or one or more additional RNA stabilizing substance as non-limiting examples, may have a dielectric constant of one or more dielectric constant in the dielectric constant list or in the dielectric constant range list.

Embodiments of the present invention may include compositions comprising one or more aprotic substance used in a composition comprising one or more RNA substance, one or more aprotic substance, and one or more of the following substances: water, cellular uptake agents, low dielectric substances, cyclic phosphate or metaphosphate containing substances, non-carbohydrate organic osmolyte substances, tertiary sulfonium containing substances, quaternary ammonium containing substances, quaternary phosphonium containing substances, hydrotrope containing substances, surfactant containing substances, betaine containing substances, stabilizing monomers, stabilizing polymers, or supplemental RNA stabilizing substances. Other embodiments may include one or more composition comprising one or more aprotic substance and one or more RNA substance and may also comprise one or more cellular uptake agent or one or more additional RNA stabilizing substances. Other embodiments may include compositions comprising one or more aprotic substance, one or more RNA substance, and one or more additional substances, such as: one or more cellular uptake agent, or one or more additional RNA stabilizing substance, or one or more buffering agent, or one or more chelating agent, or one or more inorganic salt, or water, as non-limiting examples.

Embodiments of the present invention may include one or more compositions comprising one or more RNA substance and one or more aprotic substance, wherein a composition is not lyophilized.

In one embodiment one or more composition comprising one or more RNA substance and one or more aprotic substance may not be lyophilized. In one embodiment one or more composition comprising one or more RNA substance, one or more aprotic substance, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more aprotic substance, wherein a composition may be lyophilized.

In one embodiment one or more composition comprising one or more RNA substance and one or more aprotic substance may be lyophilized. In one embodiment one or more composition comprising one or more RNA substance, one or more aprotic substance, and one or more additional substance, such as one or more cellular uptake agent or one or more additional RNA stabilizing substance as non-limiting examples, may be lyophilized.

Embodiments of the present invention may include one or more compositions comprising one or more aprotic substance and one or more RNA substance described herein, wherein one or more composition described herein may have a melting of one or more melting point in the stabilizing composition melting point list.

In one embodiment one or more composition comprising one or more RNA substance and one or more aprotic substance may have a melting point of one or more melting point in the stabilizing composition melting point list. In one embodiment one or more composition comprising one or more RNA substance, one or more aprotic substance, and one or more additional substance, such as one or more cellular uptake agent or one or more additional RNA stabilizing substance as a non-limiting example, may have a melting point of one or more melting point in the stabilizing composition melting point list.

### Low Dielectric Substances:

The inventors have discovered that low dielectric substances may stabilize RNA substances.

The inventors have discovered that RNA stabilizing substances may comprise low dielectric substances. The inventors have also discovered that the stability of RNA substances may be enhanced in an RNA storage environment comprising a low dielectric substance. The inventors have also discovered that the stability of RNA substances may be enhanced in an RNA storage environment comprising a low dielectric substance and one or more additional RNA stabilizing substance, such as an aprotic substance.

The inventors have also discovered that the stability of RNA substances may be enhanced in compositions comprising an RNA substance and a low dielectric substance. The inventors have also discovered that the stability of RNA substances may be enhanced in compositions comprising an RNA substance, a low dielectric substance, and one or more additional RNA stabilizing substance, such as an aprotic substance.

In one embodiment of the present invention, one or more RNA stabilizing substance may comprise a low dielectric substance

One embodiment of the present invention may include a composition comprised of one or more RNA substance and one or more low dielectric substance. Another embodiment of the present invention may include a composition comprised of one or more RNA substance, one or more low dielectric substance, and one or more additional RNA stabilizing substance, such as an aprotic substance.

One embodiment of the present invention may include a combination or mixture comprising one or more RNA substance and one or more low dielectric substance. Another embodiment of the present invention may include a combination or mixture comprising one or more RNA substance, one or more low dielectric substance, and one or more additional RNA stabilizing substance, such as an aprotic substance.

Embodiments of the present invention that comprise one or more RNA substance and one or more low dielectric substance may include combining, such as by mixing, one or more RNA substance with one or more substance that comprises at least one or more low dielectric substance.

Embodiments of the present invention that comprise one or more RNA substance, one or more low dielectric substance, and one or more additional RNA stabilizing substance, such as an aprotic substance, may include combining, such as by mixing, one or more RNA substance with one or more substance that comprises at least one or more low dielectric substance and one or more substance that comprises at least one or more RNA stabilizing substance, such as an aprotic substance.

An embodiment of the present invention may include compositions of materials that comprise one or more RNA substance and at least one or more low dielectric substance. The storage environment that improves the stability of RNA substances may comprise at least one or more vapor, liquid, powder, or solid low dielectric substance.

An embodiment of the present invention may include compositions of materials that comprise one or more RNA substance, one or more low dielectric substance, and one or more additional RNA stabilizing substance, such as an aprotic substance. The storage environment that improves the stability of RNA substances may comprise at least one or more vapor, liquid, powder, or solid low dielectric substance and may comprise at least one or more vapor, liquid, powder, or solid RNA stabilizing substance, such as an aprotic substance.

An embodiment of the present invention may include combinations of materials that comprise one or more RNA substance and at least one or more low dielectric substance. The storage environment that improves the stability of RNA substances may comprise at least one or more vapor, liquid, powder, or solid low dielectric substance.

An embodiment of the present invention may include combinations of materials that comprise one or more RNA substance, one or more low dielectric substance, and one or more additional RNA stabilizing substance, such as an aprotic substance. The storage environment that improves the stability of RNA substances may comprise at least one or more vapor, liquid, powder, or solid low dielectric substance and may comprise at least one or more vapor, liquid, powder, or solid RNA stabilizing substance, such as an aprotic substance.

In one embodiment of the present invention a composition comprised of one or more RNA substance and one or more low dielectric substance, produces a mixture with at least one or more RNA substance and at least one or more low dielectric substance. In one embodiment of the present invention a composition comprised of one or more RNA substance, one or more low dielectric substance, and one or more additional RNA stabilizing substance, such as an aprotic substance, produces a mixture with at least one or more RNA substance, at least one or more low dielectric substance, and at least one or more additional RNA stabilizing substance, such as an aprotic substance.

In one embodiment of the present invention a combination comprising one or more RNA substance and one or more low dielectric substance, produces a mixture with at least one or more RNA substance and at least one or more low dielectric substance. In one embodiment of the present invention a combination comprising one or more RNA substance, one or more low dielectric substance, and one or more additional RNA stabilizing substance, such as an aprotic substance, produces a mixture with at least one or more RNA substance, at least one or more low dielectric substance, and at least one or more additional RNA stabilizing substance, such as an aprotic substance.

In one embodiment of the present invention a composition with improved RNA stability may comprise one or more RNA substance and one or more low dielectric substance. In one embodiment of the present invention a composition with improved RNA stability may comprise one or more RNA substance, one or more low dielectric substance, and one or more additional RNA stabilizing substance, such as an aprotic substance.

In one embodiment of the present invention a composition with improved RNA stability may comprise a mixture of one or more RNA substance and one or more low dielectric substance. In one embodiment of the present invention a composition with improved RNA stability may comprise a mixture of one or more RNA substance, one or more low dielectric substance, and one or more additional RNA stabilizing substance, such as an aprotic substance.

In one embodiment of the present invention, each component included in a composition comprising one or more RNA substance and one or more low dielectric substance, may be stored separately, such as in a kit, or such as individual substances or as mixtures of one or more substance, and then combined later to produce a composition comprising one or more RNA substance and one or more low dielectric substance.

In one embodiment of the present invention, each component included in a composition comprising one or more RNA substance, one or more low dielectric substance, and one or more additional RNA stabilizing substance, such as an aprotic substance, may be stored separately, such as in a kit, or such as individually or as mixtures of one or more substance, and then combined later to produce a composition comprising one or more RNA substance, one or more low dielectric substance, and one or more additional RNA stabilizing substance, such as an aprotic substance.

In one embodiment of the present invention a composition comprising one or more RNA substance and one or more low dielectric substance may be at least partially biocompatible.

In one embodiment of the present invention a composition comprising one or more RNA substance, one or more low dielectric substance, and one or more additional RNA stabilizing substance, such as an aprotic substance, may be at least partially biocompatible.

In one embodiment of the present invention a combination comprised of one or more RNA substance and one or more low dielectric substance may be at least partially biocompatible.

In one embodiment of the present invention a combination comprised of one or more RNA substance, one or more low dielectric substance, and one or more additional RNA stabilizing substance, such as an aprotic substance, may be at least partially biocompatible.

Embodiments of the present invention comprising at least one or more low dielectric substances may include one or more forms of the substance. These forms may include, but are not limited to, one more counterions, ionic forms, conjugate bases, conjugate acids, protonated or deprotonated forms, hydrated or dehydrated forms, isomers, structural isomers, stereo isomers, chiral forms, salts, or combinations thereof.

A non-limiting example of a low dielectric substance that may be used is DMSO, wherein DMSO may be substituted for or used in combination with one or more low dielectric substance as described herein. One embodiment of the present invention may be a combination of substances comprising DMSO and another embodiment may also comprise at least one RNA substance. Other embodiments may be combinations of substances comprising DMSO wherein the concentration of DMSO may be one or more concentration in the stabilizing substance weight percent concentration list or in the stabilizing substance molar concentration range list or in the stabilizing substance molar concentration list.

Embodiments of the present invention may comprise one or more low dielectric substance selected from the low dielectric substance list below.

For conciseness, the following list of low dielectric substances, is herein referred to as the low dielectric substance list wherein one or more of the following substances may be substituted for or used in combination with one or more low dielectric substance as described herein.

In other embodiments, other low dielectric substances that may be substituted for or used in combination with one or more low dielectric substance as described herein may comprise one or more of the following, including but not limited to: acetone, DMSO, NMP, diethyl sulfoxide, triacetin, diethyl carbonate, dimethyl oxalate, ethyl acetate, methyl benzoate, benzyl acetate, isobutyl acetate, and butyl acetate, or derivates, or combinations thereof.

Embodiments of the present invention may include combinations comprising one or more of the substances from the low dielectric substance list substituted for or used in combination with one or more low dielectric substance or one or more RNA stabilizing substance, such as an aprotic substance as a non-limiting example, and other embodiments may also comprise at least one RNA substance.

Other embodiments may include combinations of substances comprising one or more low dielectric substance selected from the low dielectric substance list wherein the concentration of one or more substances may be one or more concentration in the stabilizing substance weight percent concentration list or in the stabilizing substance molar concentration range list or in the stabilizing substance molar concentration list.

In one embodiment of the present invention a low dielectric substance may have a dielectric constant of less than about 80 at 20° C. under standard atmospheric pressure and 1 kHz. In another embodiment of the present invention a low dielectric substance may have a dielectric constant of less than about 75 at 20° C. under standard atmospheric pressure and 1 kHz. In another embodiment of the present invention a low dielectric substance may have a dielectric constant of less than about 70 at 20° C. under standard atmospheric pressure and 1 kHz. In another embodiment of the present invention a low dielectric substance may have a dielectric constant of less than about 65 at 20° C. under standard atmospheric pressure and 1 kHz. In another embodiment of the present invention a low dielectric substance may have a dielectric constant of less than about 60 at 20° C. under standard atmospheric pressure and 1 kHz. In another embodiment of the present invention a low dielectric substance may have a dielectric constant of less than about 55 at 20° C. under standard atmospheric pressure and 1 kHz. In another embodiment of the present invention a low dielectric substance may have a dielectric constant of less than about 50 at 20° C. under standard atmospheric pressure and 1 kHz. In another embodiment of the present invention a low dielectric substance may have a dielectric constant of less than about 45 at 20° C. under standard atmospheric pressure and 1 kHz. In another embodiment of the present invention a low dielectric substance may have a dielectric constant of less than about 40 at 20° C. under standard atmospheric pressure and 1 kHz. In another embodiment of the present invention a low dielectric substance may have a dielectric constant of less than about 35 at 20° C. under standard atmospheric pressure and 1 kHz. In another embodiment of the present invention a low dielectric substance may have a dielectric constant of less than about at 20° C. under standard atmospheric pressure and 1 kHz. In another embodiment of the present invention a low dielectric substance may have a dielectric constant of less than about 25 at 20° C. under standard atmospheric pressure and 1 kHz. In another embodiment of the present invention a low dielectric substance may have a dielectric constant of less than about 20 at 20° C. under standard atmospheric pressure and 1 kHz. In another embodiment of the present invention a low dielectric substance may have a dielectric constant of less than about 15 at 20° C. under standard atmospheric pressure and 1 kHz. In another embodiment of the present invention a low dielectric substance may have a dielectric constant of less than about 10 at 20° C. under standard atmospheric pressure and 1 kHz. In another embodiment of the present invention a low dielectric substance may have a dielectric constant of less than about 9 at 20° C. under standard atmospheric pressure and 1 kHz. In another embodiment of the present invention a low dielectric substance may have a dielectric constant of less than about 8 at 20° C. under standard atmospheric pressure and 1 kHz. In another embodiment of the present invention a low dielectric substance may have a dielectric constant of less than about 7 at 20° C. under standard atmospheric pressure and 1 kHz. In another embodiment of the present invention a low dielectric substance may have a dielectric constant of less than about 6 at 20° C. under standard atmospheric pressure and 1 kHz. In another embodiment of the present invention a low dielectric substance may have a dielectric constant of less than about 5 at 20° C. under standard atmospheric pressure and 1 kHz. In another embodiment of the present invention a low dielectric substance may have a dielectric constant of less than about 4 at 20° C. under standard atmospheric pressure and 1 kHz. In another embodiment of the present invention a low dielectric substance may have a dielectric constant of less than about 3 at 20° C. under standard atmospheric pressure and 1 kHz. In another embodiment of the present invention a low dielectric substance may have a dielectric constant of less than about 2 at 20° C. under standard atmospheric pressure and 1 kHz.

In another embodiment of the present invention a low dielectric substance may have a dielectric constant between about 1-70 at 20° C. under standard atmospheric pressure and 1 kHz. In another embodiment of the present invention a low dielectric substance may have a dielectric constant between about 1-60 at 20° C. under standard atmospheric pressure and 1 kHz. In another embodiment of the present invention a low dielectric substance may have a dielectric constant between about 1-50 at 20° C. under standard atmospheric pressure and 1 kHz. In another embodiment of the present invention a low dielectric substance may have a dielectric constant between about 2-50 at 20° C. under standard atmospheric pressure and 1 kHz. In another embodiment of the present invention a low dielectric substance may have a dielectric constant between about 3-50 at 20° C. under standard atmospheric pressure and 1 kHz. In another embodiment of the present invention a low dielectric substance may have a dielectric constant between about 4-50 at 20° C. under standard atmospheric pressure and 1 kHz. In another embodiment of the present invention a low dielectric substance may have a dielectric constant between about 1-45 at 20° C. under standard atmospheric pressure and 1 kHz. In another embodiment of the present invention a low dielectric substance may have a dielectric constant between about 2-45 at 20° C. under standard atmospheric pressure and 1 kHz. In another embodiment of the present invention a low dielectric substance may have a dielectric constant between about 3-45 at 20° C. under standard atmospheric pressure and 1 kHz. In another embodiment of the present invention a low dielectric substance may have a dielectric constant between about 4-45 at 20° C. under standard atmospheric pressure and 1 kHz. In another embodiment of the present invention a low dielectric substance may have a dielectric constant between about 1-40 at 20° C. under standard atmospheric pressure and 1 kHz. In another embodiment of the present invention a low dielectric substance may have a dielectric constant between about 2-40 at 20° C. under standard atmospheric pressure and 1 kHz. In another embodiment of the present invention a low dielectric substance may have a dielectric constant between about 3-40 at 20° C. under standard atmospheric pressure and 1 kHz. In another embodiment of the present invention a low dielectric substance may have a dielectric constant between about 4-40 at 20° C. under standard atmospheric pressure and 1 kHz. In another embodiment of the present invention a low dielectric substance may have a dielectric constant between about 1-35 at 20° C. under standard atmospheric pressure and 1 kHz. In another embodiment of the present invention a low dielectric substance may have a dielectric constant between about 2-35 at 20° C. under standard atmospheric pressure and 1 kHz. In another embodiment of the present invention a low dielectric substance may have a dielectric constant between about 3-35 at 20° C. under standard atmospheric pressure and 1 kHz. In another embodiment of the present invention a low dielectric substance may have a dielectric constant between about 4-35 at 20° C. under standard atmospheric pressure and 1 kHz. In another embodiment of the present invention a low dielectric substance may have a dielectric constant between about 1-30 at 20° C. under standard atmospheric pressure and 1 kHz. In another embodiment of the present invention a low dielectric substance may have a dielectric constant between about 2-30 at 20° C. under standard atmospheric pressure and 1 kHz. In another embodiment of the present invention a low dielectric substance may have a dielectric constant between about 3-30 at 20° C. under standard atmospheric pressure and 1 kHz. In another embodiment of the present invention a low dielectric substance may have a dielectric constant between about 4-30 at 20° C. under standard atmospheric pressure and 1 kHz. In another embodiment of the present invention a low dielectric substance may have a dielectric constant between about 1-25 at 20° C. under standard atmospheric pressure and 1 kHz. In another embodiment of the present invention a low dielectric substance may have a dielectric constant between about 2-25 at 20° C. under standard atmospheric pressure and 1 kHz. In another embodiment of the present invention a low dielectric substance may have a dielectric constant between about 3-25 at 20° C. under standard atmospheric pressure and 1 kHz. In another embodiment of the present invention a low dielectric substance may have a dielectric constant between about 4-25 at 20° C. under standard atmospheric pressure and 1 kHz. In another embodiment of the present invention a low dielectric substance may have a dielectric constant between about 1-20 at 20° C. under standard atmospheric pressure and 1 kHz. In another embodiment of the present invention a low dielectric substance may have a dielectric constant between about 2-20 at 20° C. under standard atmospheric pressure and 1 kHz. In another embodiment of the present invention a low dielectric substance may have a dielectric constant between about 3-20 at 20° C. under standard atmospheric pressure and 1 kHz. In another embodiment of the present invention a low dielectric substance may have a dielectric constant between about 4-20 at 20° C. under standard atmospheric pressure and 1 kHz. In another embodiment of the present invention a low dielectric substance may have a dielectric constant between about 1-15 at 20° C. under standard atmospheric pressure and 1 kHz. In another embodiment of the present invention a low dielectric substance may have a dielectric constant between about 2-15 at 20° C. under standard atmospheric pressure and 1 kHz. In another embodiment of the present invention a low dielectric substance may have a dielectric constant between about 3-15 at 20° C. under standard atmospheric pressure and 1 kHz. In another embodiment of the present invention a low dielectric substance may have a dielectric constant between about 4-15 at 20° C. under standard atmospheric pressure and 1 kHz. In another embodiment of the present invention a low dielectric substance may have a dielectric constant between about 1-10 at 20° C. under standard atmospheric pressure and 1 kHz. In another embodiment of the present invention a low dielectric substance may have a dielectric constant between about 2-10 at 20° C. under standard atmospheric pressure and 1 kHz. In another embodiment of the present invention a low dielectric substance may have a dielectric constant between about 3-10 at 20° C. under standard atmospheric pressure and 1 kHz at 20° C. under standard atmospheric pressure and 1 kHz. In another embodiment of the present invention a low dielectric substance may have a dielectric constant between about 4-10 at 20° C. under standard atmospheric pressure and 1 kHz.

In one embodiment, a low dielectric substance may have a molecular weight between about 25-5,000 daltons. In one embodiment, a low dielectric substance may have a molecular weight between about 25-2,000 daltons. In one embodiment, a low dielectric substance may have a molecular weight between about 25-1,000 daltons. In one embodiment, a low dielectric substance may have a molecular weight between about 25-900 daltons. In one embodiment, a low dielectric substance may have a molecular weight between about 25-800 daltons. In one embodiment, a low dielectric substance may have a molecular weight between about 25-700 daltons. In one embodiment, a low dielectric substance may have a molecular weight between about 25-600 daltons. In one embodiment, a low dielectric substance may have a molecular weight between about 25-500 daltons. In one embodiment, a low dielectric substance may have a molecular weight between about 25-400 daltons. In one embodiment, a low dielectric substance may have a molecular weight between about 25-300 daltons. In one embodiment, a low dielectric substance may have a molecular weight between about 25-250 daltons. In one embodiment, a low dielectric substance may have a molecular weight between about 25-200 daltons.

In one embodiment, a low dielectric substance may have a molecular weight between about 50-5,000 daltons. In one embodiment, a low dielectric substance may have a molecular weight between about 50-2,000 daltons. In one embodiment, a low dielectric substance may have a molecular weight between about 50-1,000 daltons. In one embodiment, a low dielectric substance may have a molecular weight between about 50-900 daltons. In one embodiment, a low dielectric substance may have a molecular weight between about 50-800 daltons. In one embodiment, a low dielectric substance may have a molecular weight between about 50-700 daltons. In one embodiment, a low dielectric substance may have a molecular weight between about 50-600 daltons. In one embodiment, a low dielectric substance may have a molecular weight between about 50-500 daltons. In one embodiment, a low dielectric substance may have a molecular weight between about 50-400 daltons. In one embodiment, a low dielectric substance may have a molecular weight between about 50-300 daltons. In one embodiment, a low dielectric substance may have a molecular weight between about 50-250 daltons. In one embodiment, a low dielectric substance may have a molecular weight between about 50-200 daltons.

One embodiment of the present invention is the method whereby one or more low dielectric substance may be combined, such as by mixing, with at least one or more RNA substance to produce a mixture comprising at least one or more low dielectric substance and at least one or more RNA substance. As a non-limiting example one or more RNA substance may be mixed with one or more low dielectric substance.

Another embodiment of the present invention is the method whereby one or more low dielectric substance may be combined, such as by mixing, with at least one or more RNA substance to produce a mixture comprising at least one or more RNA substance and at least one or more low dielectric substance at one or more of the RNA stabilizing substance concentrations within the stabilizing substance molar concentration range list or in the stabilizing substance molar concentration list or the stabilizing substance weight percent concentration list as described herein. These same methods may be used to combine one or more other substances, including, but not limited to one or more cellular uptake agents or one or more additional RNA stabilizing substances, with one or more RNA substances and one or more low dielectric substances to produce a mixture comprising one or more RNA substance, one or more low dielectric substance, and one or more cellular uptake agent or one or more additional RNA stabilizing substance.

One embodiment of the present invention is the method whereby one or more low dielectric substance may be combined, such as by mixing, with at least one or more RNA substance to produce a composition comprising at least one or more RNA substance and at least one or more low dielectric substance. Another embodiment of the present invention is the method whereby one or more low dielectric substance may be combined, such as by mixing, with one or more RNA substance to produce a composition comprising at least one or more RNA substance and at least one or more low dielectric substance at one or more of the RNA stabilizing substance concentrations within the stabilizing substance molar concentration range list or in the stabilizing substance molar concentration list or stabilizing substance weight percent concentration list as described herein. These same methods may be used to combine one or more other substances, including, but not limited to one or more cellular uptake agents or one or more additional RNA stabilizing substance, with one or more RNA substance and one or more low dielectric substance to produce a composition comprising one or more RNA substance, one or more low dielectric substance, and one or more cellular uptake agent or one or more additional RNA stabilizing substance.

In one embodiment a low dielectric substance may comprise a cationic moiety at about physiologic pH. In one embodiment a low dielectric substance may comprise an anionic moiety at about physiologic pH. In one embodiment a low dielectric substance may comprise a zwitterion at about physiologic pH.

In one embodiment a low dielectric substance may comprise one or more PIF as described herein.

Embodiments of compositions comprising one or more RNA stabilizing substance may comprise different weight percent concentrations of water. For conciseness, the following list of composition weight percent concentrations of water is herein referred to as the water concentration list.

Embodiments of the present invention may include one or more compositions comprising one or more RNA substance and one or more RNA stabilizing substance, such as a low dielectric substance or an aprotic substance as non-limiting examples, wherein the composition may comprise less than about 98% water (all composition percentages herein are weight percent, unless stated otherwise). In one embodiment one or more composition comprising one or more RNA substance and one or more RNA stabilizing substance may comprise less than about 98% water. In one embodiment one or more composition comprising one or more RNA substance and one or more RNA stabilizing substance may comprise less than about 96% water. In one embodiment one or more composition comprising one or more RNA substance and one or more RNA stabilizing substance may comprise less than about 94% water. In one embodiment one or more composition comprising one or more RNA substance and one or more RNA stabilizing substance may comprise less than about 92% water. In one embodiment one or more composition comprising one or more RNA substance and one or more RNA stabilizing substance may comprise less than about 90% water. In one embodiment one or more composition comprising one or more RNA substance and one or more RNA stabilizing substance may comprise less than about 85% water. In one embodiment one or more composition comprising one or more RNA substance and one or more RNA stabilizing substance may comprise less than about 80% water. In one embodiment one or more composition comprising one or more RNA substance and one or more RNA stabilizing substance may comprise less than about 75% water. In one embodiment one or more composition comprising one or more RNA substance and one or more RNA stabilizing substance may comprise less than about 70% water. In one embodiment one or more composition comprising one or more RNA substance and one or more RNA stabilizing substance may comprise less than about 65% water. In one embodiment one or more composition comprising one or more RNA substance and one or more RNA stabilizing substance may comprise less than about 60% water. In one embodiment one or more composition comprising one or more RNA substance and one or more RNA stabilizing substance may comprise less than about 55% water. In one embodiment one or more composition comprising one or more RNA substance and one or more RNA stabilizing substance may comprise less than about 50% water. In one embodiment one or more composition comprising one or more RNA substance and one or more RNA stabilizing substance may comprise less than about 45% water. In one embodiment one or more composition comprising one or more RNA substance and one or more RNA stabilizing substance may comprise less than about 40% water. In one embodiment one or more composition comprising one or more RNA substance and one or more RNA stabilizing substance may comprise less than about 35% water. In one embodiment one or more composition comprising one or more RNA substance and one or more RNA stabilizing substance may comprise less than about 30% water. In one embodiment one or more composition comprising one or more RNA substance and one or more RNA stabilizing substance may comprise less than about 25% water. In one embodiment one or more composition comprising one or more RNA substance and one or more RNA stabilizing substance may comprise less than about 20% water. In one embodiment one or more composition comprising one or more RNA substance and one or more RNA stabilizing substance may comprise less than about 15% water. In one embodiment one or more composition comprising one or more RNA substance and one or more RNA stabilizing substance may comprise less than about 10% water.

Embodiments of compositions comprising one or more RNA stabilizing substance may comprise different weight percent concentrations of water. For conciseness, the following list of composition weight percent concentration ranges of water is herein referred to as the water concentration range list.

Embodiments of the present invention may include one or more compositions comprising one or more RNA substance and one or more RNA stabilizing substance, such as a low dielectric substance or an aprotic substance as non-limiting examples, wherein the composition may comprise between about 2% water to 98% water (all composition percentages herein are weight percent, unless stated otherwise). In one embodiment one or more composition comprising one or more RNA substance and one or more RNA stabilizing substance may comprise between about 2% water to 98% water. In one embodiment one or more composition comprising one or more RNA substance and one or more RNA stabilizing substance may comprise between about 2% water to 96% water. In one embodiment one or more composition comprising one or more RNA substance and one or more RNA stabilizing substance may comprise between about 2% water to 94% water. In one embodiment one or more composition comprising one or more RNA substance and one or more RNA stabilizing substance may comprise between about 2% water to 92% water. In one embodiment one or more composition comprising one or more RNA substance and one or more RNA stabilizing substance may comprise between about 2% water to 90% water. In one embodiment one or more composition comprising one or more RNA substance and one or more RNA stabilizing substance may comprise between about 2% water to 85% water. In one embodiment one or more composition comprising one or more RNA substance and one or more RNA stabilizing substance may comprise between about 2% water to 80% water. In one embodiment one or more composition comprising one or more RNA substance and one or more RNA stabilizing substance may comprise between about 2% water to 75% water. In one embodiment one or more composition comprising one or more RNA substance and one or more RNA stabilizing substance may comprise between about 2% water to 70% water. In one embodiment one or more composition comprising one or more RNA substance and one or more RNA stabilizing substance may comprise between about 2% water to 65% water. In one embodiment one or more composition comprising one or more RNA substance and one or more RNA stabilizing substance may comprise between about 2% water to 60% water. In one embodiment one or more composition comprising one or more RNA substance and one or more RNA stabilizing substance may comprise between about 2% water to 55% water. In one embodiment one or more composition comprising one or more RNA substance and one or more RNA stabilizing substance may comprise between about 2% water to 50% water. In one embodiment one or more composition comprising one or more RNA substance and one or more RNA stabilizing substance may comprise between about 2% water to 45% water. In one embodiment one or more composition comprising one or more RNA substance and one or more RNA stabilizing substance may comprise between about 2% water to 40% water. In one embodiment one or more composition comprising one or more RNA substance and one or more RNA stabilizing substance may comprise between about 2% water to 35% water. In one embodiment one or more composition comprising one or more RNA substance and one or more RNA stabilizing substance may comprise between about 2% water to 30% water. In one embodiment one or more composition comprising one or more RNA substance and one or more RNA stabilizing substance may comprise between about 2% water to 25% water. In one embodiment one or more composition comprising one or more RNA substance and one or more RNA stabilizing substance may comprise between about 2% water to 20% water. In one embodiment one or more composition comprising one or more RNA substance and one or more RNA stabilizing substance may comprise between about 2% water to 15% water. In one embodiment one or more composition comprising one or more RNA substance and one or more RNA stabilizing substance may comprise between about 2% water to 10% water. In one embodiment one or more composition comprising one or more RNA substance and one or more RNA stabilizing substance may comprise between about 5% water to 98% water. In one embodiment one or more composition comprising one or more RNA substance and one or more RNA stabilizing substance may comprise between about 5% water to 96% water. In one embodiment one or more composition comprising one or more RNA substance and one or more RNA stabilizing substance may comprise between about 5% water to 94% water. In one embodiment one or more composition comprising one or more RNA substance and one or more RNA stabilizing substance may comprise between about 5% water to 92% water. In one embodiment one or more composition comprising one or more RNA substance and one or more RNA stabilizing substance may comprise between about 5% water to 90% water. In one embodiment one or more composition comprising one or more RNA substance and one or more RNA stabilizing substance may comprise between about 5% water to 85% water. In one embodiment one or more composition comprising one or more RNA substance and one or more RNA stabilizing substance may comprise between about 5% water to 80% water. In one embodiment one or more composition comprising one or more RNA substance and one or more RNA stabilizing substance may comprise between about 5% water to 75% water. In one embodiment one or more composition comprising one or more RNA substance and one or more RNA stabilizing substance may comprise between about 5% water to 70% water. In one embodiment one or more composition comprising one or more RNA substance and one or more RNA stabilizing substance may comprise between about 5% water to 65% water. In one embodiment one or more composition comprising one or more RNA substance and one or more RNA stabilizing substance may comprise between about 5% water to 60% water. In one embodiment one or more composition comprising one or more RNA substance and one or more RNA stabilizing substance may comprise between about 5% water to 55% water. In one embodiment one or more composition comprising one or more RNA substance and one or more RNA stabilizing substance may comprise between about 5% water to 50% water. In one embodiment one or more composition comprising one or more RNA substance and one or more RNA stabilizing substance may comprise between about 5% water to 45% water. In one embodiment one or more composition comprising one or more RNA substance and one or more RNA stabilizing substance may comprise between about 5% water to 40% water. In one embodiment one or more composition comprising one or more RNA substance and one or more RNA stabilizing substance may comprise between about 5% water to 35% water. In one embodiment one or more composition comprising one or more RNA substance and one or more RNA stabilizing substance may comprise between about 5% water to 30% water. In one embodiment one or more composition comprising one or more RNA substance and one or more RNA stabilizing substance may comprise between about 5% water to 25% water. In one embodiment one or more composition comprising one or more RNA substance and one or more RNA stabilizing substance may comprise between about 5% water to 20% water. In one embodiment one or more composition comprising one or more RNA substance and one or more RNA stabilizing substance may comprise between about 5% water to 15% water. In one embodiment one or more composition comprising one or more RNA substance and one or more RNA stabilizing substance may comprise between about 10% water to 98% water. In one embodiment one or more composition comprising one or more RNA substance and one or more RNA stabilizing substance may comprise between about 10% water to 96% water. In one embodiment one or more composition comprising one or more RNA substance and one or more RNA stabilizing substance may comprise between about 10% water to 94% water. In one embodiment one or more composition comprising one or more RNA substance and one or more RNA stabilizing substance may comprise between about 10% water to 92% water. In one embodiment one or more composition comprising one or more RNA substance and one or more RNA stabilizing substance may comprise between about 10% water to 90% water. In one embodiment one or more composition comprising one or more RNA substance and one or more RNA stabilizing substance may comprise between about 10% water to 85% water. In one embodiment one or more composition comprising one or more RNA substance and one or more RNA stabilizing substance may comprise between about 10% water to 80% water. In one embodiment one or more composition comprising one or more RNA substance and one or more RNA stabilizing substance may comprise between about 10% water to 75% water. In one embodiment one or more composition comprising one or more RNA substance and one or more RNA stabilizing substance may comprise between about 10% water to 70% water. In one embodiment one or more composition comprising one or more RNA substance and one or more RNA stabilizing substance may comprise between about 10% water to 65% water. In one embodiment one or more composition comprising one or more RNA substance and one or more RNA stabilizing substance may comprise between about 10% water to 60% water. In one embodiment one or more composition comprising one or more RNA substance and one or more RNA stabilizing substance may comprise between about 10% water to 55% water. In one embodiment one or more composition comprising one or more RNA substance and one or more RNA stabilizing substance may comprise between about 10% water to 50% water. In one embodiment one or more composition comprising one or more RNA substance and one or more RNA stabilizing substance may comprise between about 10% water to 45% water. In one embodiment one or more composition comprising one or more RNA substance and one or more RNA stabilizing substance may comprise between about 10% water to 40% water. In one embodiment one or more composition comprising one or more RNA substance and one or more RNA stabilizing substance may comprise between about 10% water to 35% water. In one embodiment one or more composition comprising one or more RNA substance and one or more RNA stabilizing substance may comprise between about 10% water to 30% water. In one embodiment one or more composition comprising one or more RNA substance and one or more RNA stabilizing substance may comprise between about 10% water to 25% water. In one embodiment one or more composition comprising one or more RNA substance and one or more RNA stabilizing substance may comprise between about 10% water to 20% water. In one embodiment one or more composition comprising one or more RNA substance and one or more RNA stabilizing substance may comprise between about 15% water to 98% water. In one embodiment one or more composition comprising one or more RNA substance and one or more RNA stabilizing substance may comprise between about 15% water to 96% water. In one embodiment one or more composition comprising one or more RNA substance and one or more RNA stabilizing substance may comprise between about 15% water to 94% water. In one embodiment one or more composition comprising one or more RNA substance and one or more RNA stabilizing substance may comprise between about 15% water to 92% water. In one embodiment one or more composition comprising one or more RNA substance and one or more RNA stabilizing substance may comprise between about 15% water to 90% water. In one embodiment one or more composition comprising one or more RNA substance and one or more RNA stabilizing substance may comprise between about 15% water to 85% water. In one embodiment one or more composition comprising one or more RNA substance and one or more RNA stabilizing substance may comprise between about 15% water to 80% water. In one embodiment one or more composition comprising one or more RNA substance and one or more RNA stabilizing substance may comprise between about 15% water to 75% water. In one embodiment one or more composition comprising one or more RNA substance and one or more RNA stabilizing substance may comprise between about 15% water to 70% water. In one embodiment one or more composition comprising one or more RNA substance and one or more RNA stabilizing substance may comprise between about 15% water to 65% water. In one embodiment one or more composition comprising one or more RNA substance and one or more RNA stabilizing substance may comprise between about 15% water to 60% water. In one embodiment one or more composition comprising one or more RNA substance and one or more RNA stabilizing substance may comprise between about 15% water to 55% water. In one embodiment one or more composition comprising one or more RNA substance and one or more RNA stabilizing substance may comprise between about 15% water to 50% water. In one embodiment one or more composition comprising one or more RNA substance and one or more RNA stabilizing substance may comprise between about 15% water to 45% water. In one embodiment one or more composition comprising one or more RNA substance and one or more RNA stabilizing substance may comprise between about 15% water to 40% water. In one embodiment one or more composition comprising one or more RNA substance and one or more RNA stabilizing substance may comprise between about 15% water to 35% water. In one embodiment one or more composition comprising one or more RNA substance and one or more RNA stabilizing substance may comprise between about 15% water to 30% water. In one embodiment one or more composition comprising one or more RNA substance and one or more RNA stabilizing substance may comprise between about 15% water to 25% water. In one embodiment one or more composition comprising one or more RNA substance and one or more RNA stabilizing substance may comprise between about 20% water to 98% water. In one embodiment one or more composition comprising one or more RNA substance and one or more RNA stabilizing substance may comprise between about 20% water to 96% water. In one embodiment one or more composition comprising one or more RNA substance and one or more RNA stabilizing substance may comprise between about 20% water to 94% water. In one embodiment one or more composition comprising one or more RNA substance and one or more RNA stabilizing substance may comprise between about 20% water to 92% water. In one embodiment one or more composition comprising one or more RNA substance and one or more RNA stabilizing substance may comprise between about 20% water to 90% water. In one embodiment one or more composition comprising one or more RNA substance and one or more RNA stabilizing substance may comprise between about 20% water to 85% water. In one embodiment one or more composition comprising one or more RNA substance and one or more RNA stabilizing substance may comprise between about 20% water to 80% water. In one embodiment one or more composition comprising one or more RNA substance and one or more RNA stabilizing substance may comprise between about 20% water to 75% water. In one embodiment one or more composition comprising one or more RNA substance and one or more RNA stabilizing substance may comprise between about 20% water to 70% water. In one embodiment one or more composition comprising one or more RNA substance and one or more RNA stabilizing substance may comprise between about 20% water to 65% water. In one embodiment one or more composition comprising one or more RNA substance and one or more RNA stabilizing substance may comprise between about 20% water to 60% water. In one embodiment one or more composition comprising one or more RNA substance and one or more RNA stabilizing substance may comprise between about 20% water to 55% water. In one embodiment one or more composition comprising one or more RNA substance and one or more RNA stabilizing substance may comprise between about 20% water to 50% water. In one embodiment one or more composition comprising one or more RNA substance and one or more RNA stabilizing substance may comprise between about 20% water to 45% water. In one embodiment one or more composition comprising one or more RNA substance and one or more RNA stabilizing substance may comprise between about 20% water to 40% water. In one embodiment one or more composition comprising one or more RNA substance and one or more RNA stabilizing substance may comprise between about 20% water to 35% water. In one embodiment one or more composition comprising one or more RNA substance and one or more RNA stabilizing substance may comprise between about 20% water to 30% water. In one embodiment one or more composition comprising one or more RNA substance and one or more RNA stabilizing substance may comprise between about 25% water to 98% water. In one embodiment one or more composition comprising one or more RNA substance and one or more RNA stabilizing substance may comprise between about 25% water to 96% water. In one embodiment one or more composition comprising one or more RNA substance and one or more RNA stabilizing substance may comprise between about 25% water to 94% water. In one embodiment one or more composition comprising one or more RNA substance and one or more RNA stabilizing substance may comprise between about 25% water to 92% water. In one embodiment one or more composition comprising one or more RNA substance and one or more RNA stabilizing substance may comprise between about 25% water to 90% water. In one embodiment one or more composition comprising one or more RNA substance and one or more RNA stabilizing substance may comprise between about 25% water to 85% water. In one embodiment one or more composition comprising one or more RNA substance and one or more RNA stabilizing substance may comprise between about 25% water to 80% water. In one embodiment one or more composition comprising one or more RNA substance and one or more RNA stabilizing substance may comprise between about 25% water to 75% water. In one embodiment one or more composition comprising one or more RNA substance and one or more RNA stabilizing substance may comprise between about 25% water to 70% water. In one embodiment one or more composition comprising one or more RNA substance and one or more RNA stabilizing substance may comprise between about 25% water to 65% water. In one embodiment one or more composition comprising one or more RNA substance and one or more RNA stabilizing substance may comprise between about 25% water to 60% water. In one embodiment one or more composition comprising one or more RNA substance and one or more RNA stabilizing substance may comprise between about 25% water to 55% water. In one embodiment one or more composition comprising one or more RNA substance and one or more RNA stabilizing substance may comprise between about 25% water to 50% water. In one embodiment one or more composition comprising one or more RNA substance and one or more RNA stabilizing substance may comprise between about 25% water to 45% water. In one embodiment one or more composition comprising one or more RNA substance and one or more RNA stabilizing substance may comprise between about 25% water to 40% water. In one embodiment one or more composition comprising one or more RNA substance and one or more RNA stabilizing substance may comprise between about 25% water to 35% water. In one embodiment one or more composition comprising one or more RNA substance and one or more RNA stabilizing substance may comprise between about 30% water to 98% water. In one embodiment one or more composition comprising one or more RNA substance and one or more RNA stabilizing substance may comprise between about 30% water to 96% water. In one embodiment one or more composition comprising one or more RNA substance and one or more RNA stabilizing substance may comprise between about 30% water to 94% water. In one embodiment one or more composition comprising one or more RNA substance and one or more RNA stabilizing substance may comprise between about 30% water to 92% water. In one embodiment one or more composition comprising one or more RNA substance and one or more RNA stabilizing substance may comprise between about 30% water to 90% water. In one embodiment one or more composition comprising one or more RNA substance and one or more RNA stabilizing substance may comprise between about 30% water to 85% water. In one embodiment one or more composition comprising one or more RNA substance and one or more RNA stabilizing substance may comprise between about 30% water to 80% water. In one embodiment one or more composition comprising one or more RNA substance and one or more RNA stabilizing substance may comprise between about 30% water to 75% water. In one embodiment one or more composition comprising one or more RNA substance and one or more RNA stabilizing substance may comprise between about 30% water to 70% water. In one embodiment one or more composition comprising one or more RNA substance and one or more RNA stabilizing substance may comprise between about 30% water to 65% water. In one embodiment one or more composition comprising one or more RNA substance and one or more RNA stabilizing substance may comprise between about 30% water to 60% water. In one embodiment one or more composition comprising one or more RNA substance and one or more RNA stabilizing substance may comprise between about 30% water to 55% water. In one embodiment one or more composition comprising one or more RNA substance and one or more RNA stabilizing substance may comprise between about 30% water to 50% water. In one embodiment one or more composition comprising one or more RNA substance and one or more RNA stabilizing substance may comprise between about 30% water to 45% water. In one embodiment one or more composition comprising one or more RNA substance and one or more RNA stabilizing substance may comprise between about 30% water to 40% water. In one embodiment one or more composition comprising one or more RNA substance and one or more RNA stabilizing substance may comprise between about 40% water to 98% water. In one embodiment one or more composition comprising one or more RNA substance and one or more RNA stabilizing substance may comprise between about 40% water to 96% water. In one embodiment one or more composition comprising one or more RNA substance and one or more RNA stabilizing substance may comprise between about 40% water to 94% water. In one embodiment one or more composition comprising one or more RNA substance and one or more RNA stabilizing substance may comprise between about 40% water to 92% water. In one embodiment one or more composition comprising one or more RNA substance and one or more RNA stabilizing substance may comprise between about 40% water to 90% water. In one embodiment one or more composition comprising one or more RNA substance and one or more RNA stabilizing substance may comprise between about 40% water to 85% water. In one embodiment one or more composition comprising one or more RNA substance and one or more RNA stabilizing substance may comprise between about 40% water to 80% water. In one embodiment one or more composition comprising one or more RNA substance and one or more RNA stabilizing substance may comprise between about 40% water to 75% water. In one embodiment one or more composition comprising one or more RNA substance and one or more RNA stabilizing substance may comprise between about 40% water to 70% water. In one embodiment one or more composition comprising one or more RNA substance and one or more RNA stabilizing substance may comprise between about 40% water to 65% water. In one embodiment one or more composition comprising one or more RNA substance and one or more RNA stabilizing substance may comprise between about 40% water to 60% water. In one embodiment one or more composition comprising one or more RNA substance and one or more RNA stabilizing substance may comprise between about 40% water to 55% water. In one embodiment one or more composition comprising one or more RNA substance and one or more RNA stabilizing substance may comprise between about 40% water to 50% water. In one embodiment one or more composition comprising one or more RNA substance and one or more RNA stabilizing substance may comprise between about 50% water to 98% water. In one embodiment one or more composition comprising one or more RNA substance and one or more RNA stabilizing substance may comprise between about 50% water to 96% water. In one embodiment one or more composition comprising one or more RNA substance and one or more RNA stabilizing substance may comprise between about 50% water to 94% water. In one embodiment one or more composition comprising one or more RNA substance and one or more RNA stabilizing substance may comprise between about 50% water to 92% water. In one embodiment one or more composition comprising one or more RNA substance and one or more RNA stabilizing substance may comprise between about 50% water to 90% water. In one embodiment one or more composition comprising one or more RNA substance and one or more RNA stabilizing substance may comprise between about 50% water to 85% water. In one embodiment one or more composition comprising one or more RNA substance and one or more RNA stabilizing substance may comprise between about 50% water to 80% water. In one embodiment one or more composition comprising one or more RNA substance and one or more RNA stabilizing substance may comprise between about 50% water to 75% water. In one embodiment one or more composition comprising one or more RNA substance and one or more RNA stabilizing substance may comprise between about 50% water to 70% water. In one embodiment one or more composition comprising one or more RNA substance and one or more RNA stabilizing substance may comprise between about 50% water to 65% water. In one embodiment one or more composition comprising one or more RNA substance and one or more RNA stabilizing substance may comprise between about 50% water to 60% water.

Embodiments of compositions comprising one or more RNA stabilizing substance may have different dielectric constants. For conciseness, the following list of composition dielectric constants is herein referred to as the composition dielectric constant list.

Embodiments of the present invention may include one or more compositions comprising one or more RNA substance and one or more RNA stabilizing substance, such as a low dielectric substance or an aprotic substance as non-limiting examples, wherein the composition may have a dielectric constant of less than about 80 at 20° C. under standard atmospheric pressure and 1 kHz. In one embodiment one or more composition comprising one or more RNA substance and one or more RNA stabilizing substance may have a dielectric constant of less than about 80 at 20° C. under standard atmospheric pressure and 1 kHz. In one embodiment one or more composition comprising one or more RNA substance and one or more RNA stabilizing substance may have a dielectric constant of less than about 75 at 20° C. under standard atmospheric pressure and 1 kHz. In one embodiment one or more composition comprising one or more RNA substance and one or more RNA stabilizing substance may have a dielectric constant of less than about 70 at 20° C. under standard atmospheric pressure and 1 kHz. In one embodiment one or more composition comprising one or more RNA substance and one or more RNA stabilizing substance may have a dielectric constant of less than about 65 at 20° C. under standard atmospheric pressure and 1 kHz. In one embodiment one or more composition comprising one or more RNA substance and one or more RNA stabilizing substance may have a dielectric constant of less than about 60 at 20° C. under standard atmospheric pressure and 1 kHz. In one embodiment one or more composition comprising one or more RNA substance and one or more RNA stabilizing substance may have a dielectric constant of less than about 55 at 20° C. under standard atmospheric pressure and 1 kHz. In one embodiment one or more composition comprising one or more RNA substance and one or more RNA stabilizing substance may have a dielectric constant of less than about 50 at 20° C. under standard atmospheric pressure and 1 kHz. In one embodiment one or more composition comprising one or more RNA substance and one or more RNA stabilizing substance may have a dielectric constant of less than about 45 at 20° C. under standard atmospheric pressure and 1 kHz. In one embodiment one or more composition comprising one or more RNA substance and one or more RNA stabilizing substance may have a dielectric constant of less than about 40 at 20° C. under standard atmospheric pressure and 1 kHz. In one embodiment one or more composition comprising one or more RNA substance and one or more RNA stabilizing substance may have a dielectric constant of less than about 35 at 20° C. under standard atmospheric pressure and 1 kHz. In one embodiment one or more composition comprising one or more RNA substance and one or more RNA stabilizing substance may have a dielectric constant of less than about 30 at 20° C. under standard atmospheric pressure and 1 kHz. In one embodiment one or more composition comprising one or more RNA substance and one or more RNA stabilizing substance may have a dielectric constant of less than about 25 at 20° C. under standard atmospheric pressure and 1 kHz. In one embodiment one or more composition comprising one or more RNA substance and one or more RNA stabilizing substance may have a dielectric constant of less than about 20 at 20° C. under standard atmospheric pressure and 1 kHz. In one embodiment one or more composition comprising one or more RNA substance and one or more RNA stabilizing substance may have a dielectric constant of less than about 15 at 20° C. under standard atmospheric pressure and 1 kHz. In one embodiment one or more composition comprising one or more RNA substance and one or more RNA stabilizing substance may have a dielectric constant of less than about 10 at 20° C. under standard atmospheric pressure and 1 kHz. In one embodiment one or more composition comprising one or more RNA substance and one or more RNA stabilizing substance may have a dielectric constant of less than about 9 at 20° C. under standard atmospheric pressure and 1 kHz. In one embodiment one or more composition comprising one or more RNA substance and one or more RNA stabilizing substance may have a dielectric constant of less than about 8 at 20° C. under standard atmospheric pressure and 1 kHz. In one embodiment one or more composition comprising one or more RNA substance and one or more RNA stabilizing substance may have a dielectric constant of less than about 7 at 20° C. under standard atmospheric pressure and 1 kHz. In one embodiment one or more composition comprising one or more RNA substance and one or more RNA stabilizing substance may have a dielectric constant of less than about 6 at 20° C. under standard atmospheric pressure and 1 kHz. In one embodiment one or more composition comprising one or more RNA substance and one or more RNA stabilizing substance may have a dielectric constant of less than about 5 at 20° C. under standard atmospheric pressure and 1 kHz. In one embodiment one or more composition comprising one or more RNA substance and one or more RNA stabilizing substance may have a dielectric constant of less than about 4 at 20° C. under standard atmospheric pressure and 1 kHz. In one embodiment one or more composition comprising one or more RNA substance and one or more RNA stabilizing substance may have a dielectric constant of less than about 3 at 20° C. under standard atmospheric pressure and 1 kHz. In one embodiment one or more composition comprising one or more RNA substance and one or more RNA stabilizing substance may have a dielectric constant of less than about 2 at 20° C. under standard atmospheric pressure and 1 kHz.

Embodiments of compositions comprising one or more RNA stabilizing substance may have different dielectric constants. For conciseness, the following list of composition dielectric constant ranges is herein referred to as the composition dielectric constant range list.

Embodiments of the present invention may include one or more compositions comprising one or more RNA substance and one or more RNA stabilizing substance, such as a low dielectric substance or an aprotic substance as non-limiting examples, wherein the composition may have a dielectric constant between about 1-80 at 20° C. under standard atmospheric pressure and 1 kHz. In one embodiment one or more composition comprising one or more RNA substance and one or more RNA stabilizing substance may have a dielectric constant between about 1-80 at 20° C. under standard atmospheric pressure and 1 kHz. In one embodiment one or more composition comprising one or more RNA substance and one or more RNA stabilizing substance may have a dielectric constant between about 2-80 at 20° C. under standard atmospheric pressure and 1 kHz. In one embodiment one or more composition comprising one or more RNA substance and one or more RNA stabilizing substance may have a dielectric constant between about 3-80 at 20° C. under standard atmospheric pressure and 1 kHz. In one embodiment one or more composition comprising one or more RNA substance and one or more RNA stabilizing substance may have a dielectric constant between about 4-80 at 20° C. under standard atmospheric pressure and 1 kHz. In one embodiment one or more composition comprising one or more RNA substance and one or more RNA stabilizing substance may have a dielectric constant between about 5-80 at 20° C. under standard atmospheric pressure and 1 kHz. In one embodiment one or more composition comprising one or more RNA substance and one or more RNA stabilizing substance may have a dielectric constant between about 10-80 at 20° C. under standard atmospheric pressure and 1 kHz. In one embodiment one or more composition comprising one or more RNA substance and one or more RNA stabilizing substance may have a dielectric constant between about 15-80 at 20° C. under standard atmospheric pressure and 1 kHz. In one embodiment one or more composition comprising one or more RNA substance and one or more RNA stabilizing substance may have a dielectric constant between about 20-80 at 20° C. under standard atmospheric pressure and 1 kHz. In one embodiment one or more composition comprising one or more RNA substance and one or more RNA stabilizing substance may have a dielectric constant between about 25-80 at 20° C. under standard atmospheric pressure and 1 kHz. In one embodiment one or more composition comprising one or more RNA substance and one or more RNA stabilizing substance may have a dielectric constant between about 30-80 at 20° C. under standard atmospheric pressure and 1 kHz. In one embodiment one or more composition comprising one or more RNA substance and one or more RNA stabilizing substance may have a dielectric constant between about 35-80 at 20° C. under standard atmospheric pressure and 1 kHz. In one embodiment one or more composition comprising one or more RNA substance and one or more RNA stabilizing substance may have a dielectric constant between about 40-80 at 20° C. under standard atmospheric pressure and 1 kHz. In one embodiment one or more composition comprising one or more RNA substance and one or more RNA stabilizing substance may have a dielectric constant between about 45-80 at 20° C. under standard atmospheric pressure and 1 kHz. In one embodiment one or more composition comprising one or more RNA substance and one or more RNA stabilizing substance may have a dielectric constant between about 50-80 at 20° C. under standard atmospheric pressure and 1 kHz. In one embodiment one or more composition comprising one or more RNA substance and one or more RNA stabilizing substance may have a dielectric constant between about 1-70 at 20° C. under standard atmospheric pressure and 1 kHz. In one embodiment one or more composition comprising one or more RNA substance and one or more RNA stabilizing substance may have a dielectric constant between about 2-70 at 20° C. under standard atmospheric pressure and 1 kHz. In one embodiment one or more composition comprising one or more RNA substance and one or more RNA stabilizing substance may have a dielectric constant between about 3-70 at 20° C. under standard atmospheric pressure and 1 kHz. In one embodiment one or more composition comprising one or more RNA substance and one or more RNA stabilizing substance may have a dielectric constant between about 4-70 at 20° C. under standard atmospheric pressure and 1 kHz. In one embodiment one or more composition comprising one or more RNA substance and one or more RNA stabilizing substance may have a dielectric constant between about 5-70 at 20° C. under standard atmospheric pressure and 1 kHz. In one embodiment one or more composition comprising one or more RNA substance and one or more RNA stabilizing substance may have a dielectric constant between about 10-70 at 20° C. under standard atmospheric pressure and 1 kHz. In one embodiment one or more composition comprising one or more RNA substance and one or more RNA stabilizing substance may have a dielectric constant between about 15-70 at 20° C. under standard atmospheric pressure and 1 kHz. In one embodiment one or more composition comprising one or more RNA substance and one or more RNA stabilizing substance may have a dielectric constant between about 20-70 at 20° C. under standard atmospheric pressure and 1 kHz. In one embodiment one or more composition comprising one or more RNA substance and one or more RNA stabilizing substance may have a dielectric constant between about 25-70 at 20° C. under standard atmospheric pressure and 1 kHz. In one embodiment one or more composition comprising one or more RNA substance and one or more RNA stabilizing substance may have a dielectric constant between about 30-70 at 20° C. under standard atmospheric pressure and 1 kHz. In one embodiment one or more composition comprising one or more RNA substance and one or more RNA stabilizing substance may have a dielectric constant between about 35-70 at 20° C. under standard atmospheric pressure and 1 kHz. In one embodiment one or more composition comprising one or more RNA substance and one or more RNA stabilizing substance may have a dielectric constant between about 40-70 at 20° C. under standard atmospheric pressure and 1 kHz. In one embodiment one or more composition comprising one or more RNA substance and one or more RNA stabilizing substance may have a dielectric constant between about 45-70 at 20° C. under standard atmospheric pressure and 1 kHz. In one embodiment one or more composition comprising one or more RNA substance and one or more RNA stabilizing substance may have a dielectric constant between about 50-70 at 20° C. under standard atmospheric pressure and 1 kHz. In one embodiment one or more composition comprising one or more RNA substance and one or more RNA stabilizing substance may have a dielectric constant between about 1-60 at 20° C. under standard atmospheric pressure and 1 kHz. In one embodiment one or more composition comprising one or more RNA substance and one or more RNA stabilizing substance may have a dielectric constant between about 2-60 at 20° C. under standard atmospheric pressure and 1 kHz. In one embodiment one or more composition comprising one or more RNA substance and one or more RNA stabilizing substance may have a dielectric constant between about 3-60 at 20° C. under standard atmospheric pressure and 1 kHz. In one embodiment one or more composition comprising one or more RNA substance and one or more RNA stabilizing substance may have a dielectric constant between about 4-60 at 20° C. under standard atmospheric pressure and 1 kHz. In one embodiment one or more composition comprising one or more RNA substance and one or more RNA stabilizing substance may have a dielectric constant between about 5-60 at 20° C. under standard atmospheric pressure and 1 kHz. In one embodiment one or more composition comprising one or more RNA substance and one or more RNA stabilizing substance may have a dielectric constant between about 10-60 at 20° C. under standard atmospheric pressure and 1 kHz. In one embodiment one or more composition comprising one or more RNA substance and one or more RNA stabilizing substance may have a dielectric constant between about 15-60 at 20° C. under standard atmospheric pressure and 1 kHz. In one embodiment one or more composition comprising one or more RNA substance and one or more RNA stabilizing substance may have a dielectric constant between about 20-60 at 20° C. under standard atmospheric pressure and 1 kHz. In one embodiment one or more composition comprising one or more RNA substance and one or more RNA stabilizing substance may have a dielectric constant between about 25-60 at 20° C. under standard atmospheric pressure and 1 kHz. In one embodiment one or more composition comprising one or more RNA substance and one or more RNA stabilizing substance may have a dielectric constant between about 30-60 at 20° C. under standard atmospheric pressure and 1 kHz. In one embodiment one or more composition comprising one or more RNA substance and one or more RNA stabilizing substance may have a dielectric constant between about 35-60 at 20° C. under standard atmospheric pressure and 1 kHz. In one embodiment one or more composition comprising one or more RNA substance and one or more RNA stabilizing substance may have a dielectric constant between about 40-60 at 20° C. under standard atmospheric pressure and 1 kHz. In one embodiment one or more composition comprising one or more RNA substance and one or more RNA stabilizing substance may have a dielectric constant between about 45-60 at 20° C. under standard atmospheric pressure and 1 kHz. In one embodiment one or more composition comprising one or more RNA substance and one or more RNA stabilizing substance may have a dielectric constant between about 50-60 at 20° C. under standard atmospheric pressure and 1 kHz. In one embodiment one or more composition comprising one or more RNA substance and one or more RNA stabilizing substance may have a dielectric constant between about 1-50 at 20° C. under standard atmospheric pressure and 1 kHz. In one embodiment one or more composition comprising one or more RNA substance and one or more RNA stabilizing substance may have a dielectric constant between about 2-50 at 20° C. under standard atmospheric pressure and 1 kHz. In one embodiment one or more composition comprising one or more RNA substance and one or more RNA stabilizing substance may have a dielectric constant between about 3-50 at 20° C. under standard atmospheric pressure and 1 kHz. In one embodiment one or more composition comprising one or more RNA substance and one or more RNA stabilizing substance may have a dielectric constant between about 4-50 at 20° C. under standard atmospheric pressure and 1 kHz. In one embodiment one or more composition comprising one or more RNA substance and one or more RNA stabilizing substance may have a dielectric constant between about 5-50 at 20° C. under standard atmospheric pressure and 1 kHz. In one embodiment one or more composition comprising one or more RNA substance and one or more RNA stabilizing substance may have a dielectric constant between about 10-50 at 20° C. under standard atmospheric pressure and 1 kHz. In one embodiment one or more composition comprising one or more RNA substance and one or more RNA stabilizing substance may have a dielectric constant between about 15-50 at 20° C. under standard atmospheric pressure and 1 kHz. In one embodiment one or more composition comprising one or more RNA substance and one or more RNA stabilizing substance may have a dielectric constant between about 20-50 at 20° C. under standard atmospheric pressure and 1 kHz. In one embodiment one or more composition comprising one or more RNA substance and one or more RNA stabilizing substance may have a dielectric constant between about 25-50 at 20° C. under standard atmospheric pressure and 1 kHz. In one embodiment one or more composition comprising one or more RNA substance and one or more RNA stabilizing substance may have a dielectric constant between about 30-50 at 20° C. under standard atmospheric pressure and 1 kHz. In one embodiment one or more composition comprising one or more RNA substance and one or more RNA stabilizing substance may have a dielectric constant between about 35-50 at 20° C. under standard atmospheric pressure and 1 kHz. In one embodiment one or more composition comprising one or more RNA substance and one or more RNA stabilizing substance may have a dielectric constant between about 40-50 at 20° C. under standard atmospheric pressure and 1 kHz. In one embodiment one or more composition comprising one or more RNA substance and one or more RNA stabilizing substance may have a dielectric constant between about 1-45 at 20° C. under standard atmospheric pressure and 1 kHz. In one embodiment one or more composition comprising one or more RNA substance and one or more RNA stabilizing substance may have a dielectric constant between about 2-45 at 20° C. under standard atmospheric pressure and 1 kHz. In one embodiment one or more composition comprising one or more RNA substance and one or more RNA stabilizing substance may have a dielectric constant between about 3-45 at 20° C. under standard atmospheric pressure and 1 kHz. In one embodiment one or more composition comprising one or more RNA substance and one or more RNA stabilizing substance may have a dielectric constant between about 4-45 at 20° C. under standard atmospheric pressure and 1 kHz. In one embodiment one or more composition comprising one or more RNA substance and one or more RNA stabilizing substance may have a dielectric constant between about 5-45 at 20° C. under standard atmospheric pressure and 1 kHz. In one embodiment one or more composition comprising one or more RNA substance and one or more RNA stabilizing substance may have a dielectric constant between about 10-45 at 20° C. under standard atmospheric pressure and 1 kHz. In one embodiment one or more composition comprising one or more RNA substance and one or more RNA stabilizing substance may have a dielectric constant between about 15-45 at 20° C. under standard atmospheric pressure and 1 kHz. In one embodiment one or more composition comprising one or more RNA substance and one or more RNA stabilizing substance may have a dielectric constant between about 20-45 at 20° C. under standard atmospheric pressure and 1 kHz. In one embodiment one or more composition comprising one or more RNA substance and one or more RNA stabilizing substance may have a dielectric constant between about 25-45 at 20° C. under standard atmospheric pressure and 1 kHz. In one embodiment one or more composition comprising one or more RNA substance and one or more RNA stabilizing substance may have a dielectric constant between about 30-45 at 20° C. under standard atmospheric pressure and 1 kHz. In one embodiment one or more composition comprising one or more RNA substance and one or more RNA stabilizing substance may have a dielectric constant between about 35-45 at 20° C. under standard atmospheric pressure and 1 kHz. In one embodiment one or more composition comprising one or more RNA substance and one or more RNA stabilizing substance may have a dielectric constant between about 1-40 at 20° C. under standard atmospheric pressure and 1 kHz. In one embodiment one or more composition comprising one or more RNA substance and one or more RNA stabilizing substance may have a dielectric constant between about 2-40 at 20° C. under standard atmospheric pressure and 1 kHz. In one embodiment one or more composition comprising one or more RNA substance and one or more RNA stabilizing substance may have a dielectric constant between about 3-40 at 20° C. under standard atmospheric pressure and 1 kHz. In one embodiment one or more composition comprising one or more RNA substance and one or more RNA stabilizing substance may have a dielectric constant between about 4-40 at 20° C. under standard atmospheric pressure and 1 kHz. In one embodiment one or more composition comprising one or more RNA substance and one or more RNA stabilizing substance may have a dielectric constant between about 5-40 at 20° C. under standard atmospheric pressure and 1 kHz. In one embodiment one or more composition comprising one or more RNA substance and one or more RNA stabilizing substance may have a dielectric constant between about 10-40 at 20° C. under standard atmospheric pressure and 1 kHz. In one embodiment one or more composition comprising one or more RNA substance and one or more RNA stabilizing substance may have a dielectric constant between about 15-40 at 20° C. under standard atmospheric pressure and 1 kHz. In one embodiment one or more composition comprising one or more RNA substance and one or more RNA stabilizing substance may have a dielectric constant between about 20-40 at 20° C. under standard atmospheric pressure and 1 kHz. In one embodiment one or more composition comprising one or more RNA substance and one or more RNA stabilizing substance may have a dielectric constant between about 25-40 at 20° C. under standard atmospheric pressure and 1 kHz. In one embodiment one or more composition comprising one or more RNA substance and one or more RNA stabilizing substance may have a dielectric constant between about 30-40 at 20° C. under standard atmospheric pressure and 1 kHz. In one embodiment one or more composition comprising one or more RNA substance and one or more RNA stabilizing substance may have a dielectric constant between about 1-35 at 20° C. under standard atmospheric pressure and 1 kHz. In one embodiment one or more composition comprising one or more RNA substance and one or more RNA stabilizing substance may have a dielectric constant between about 2-35 at 20° C. under standard atmospheric pressure and 1 kHz. In one embodiment one or more composition comprising one or more RNA substance and one or more RNA stabilizing substance may have a dielectric constant between about 3-35 at 20° C. under standard atmospheric pressure and 1 kHz. In one embodiment one or more composition comprising one or more RNA substance and one or more RNA stabilizing substance may have a dielectric constant between about 4-35 at 20° C. under standard atmospheric pressure and 1 kHz. In one embodiment one or more composition comprising one or more RNA substance and one or more RNA stabilizing substance may have a dielectric constant between about 5-35 at 20° C. under standard atmospheric pressure and 1 kHz. In one embodiment one or more composition comprising one or more RNA substance and one or more RNA stabilizing substance may have a dielectric constant between about 10-35 at 20° C. under standard atmospheric pressure and 1 kHz. In one embodiment one or more composition comprising one or more RNA substance and one or more RNA stabilizing substance may have a dielectric constant between about 15-35 at 20° C. under standard atmospheric pressure and 1 kHz. In one embodiment one or more composition comprising one or more RNA substance and one or more RNA stabilizing substance may have a dielectric constant between about 20-35 at 20° C. under standard atmospheric pressure and 1 kHz. In one embodiment one or more composition comprising one or more RNA substance and one or more RNA stabilizing substance may have a dielectric constant between about 25-35 at 20° C. under standard atmospheric pressure and 1 kHz. In one embodiment one or more composition comprising one or more RNA substance and one or more RNA stabilizing substance may have a dielectric constant between about 1-30 at 20° C. under standard atmospheric pressure and 1 kHz. In one embodiment one or more composition comprising one or more RNA substance and one or more RNA stabilizing substance may have a dielectric constant between about 2-30 at 20° C. under standard atmospheric pressure and 1 kHz. In one embodiment one or more composition comprising one or more RNA substance and one or more RNA stabilizing substance may have a dielectric constant between about 3-30 at 20° C. under standard atmospheric pressure and 1 kHz. In one embodiment one or more composition comprising one or more RNA substance and one or more RNA stabilizing substance may have a dielectric constant between about 4-30 at 20° C. under standard atmospheric pressure and 1 kHz. In one embodiment one or more composition comprising one or more RNA substance and one or more RNA stabilizing substance may have a dielectric constant between about 5-30 at 20° C. under standard atmospheric pressure and 1 kHz. In one embodiment one or more composition comprising one or more RNA substance and one or more RNA stabilizing substance may have a dielectric constant between about 10-30 at 20° C. under standard atmospheric pressure and 1 kHz. In one embodiment one or more composition comprising one or more RNA substance and one or more RNA stabilizing substance may have a dielectric constant between about 15-30 at 20° C. under standard atmospheric pressure and 1 kHz. In one embodiment one or more composition comprising one or more RNA substance and one or more RNA stabilizing substance may have a dielectric constant between about 20-30 at 20° C. under standard atmospheric pressure and 1 kHz. In one embodiment one or more composition comprising one or more RNA substance and one or more RNA stabilizing substance may have a dielectric constant between about 1-25 at 20° C. under standard atmospheric pressure and 1 kHz. In one embodiment one or more composition comprising one or more RNA substance and one or more RNA stabilizing substance may have a dielectric constant between about 2-25 at 20° C. under standard atmospheric pressure and 1 kHz. In one embodiment one or more composition comprising one or more RNA substance and one or more RNA stabilizing substance may have a dielectric constant between about 3-25 at 20° C. under standard atmospheric pressure and 1 kHz. In one embodiment one or more composition comprising one or more RNA substance and one or more RNA stabilizing substance may have a dielectric constant between about 4-25 at 20° C. under standard atmospheric pressure and 1 kHz. In one embodiment one or more composition comprising one or more RNA substance and one or more RNA stabilizing substance may have a dielectric constant between about 5-25 at 20° C. under standard atmospheric pressure and 1 kHz. In one embodiment one or more composition comprising one or more RNA substance and one or more RNA stabilizing substance may have a dielectric constant between about 10-25 at 20° C. under standard atmospheric pressure and 1 kHz. In one embodiment one or more composition comprising one or more RNA substance and one or more RNA stabilizing substance may have a dielectric constant between about 15-25 at 20° C. under standard atmospheric pressure and 1 kHz. In one embodiment one or more composition comprising one or more RNA substance and one or more RNA stabilizing substance may have a dielectric constant between about 1-20 at 20° C. under standard atmospheric pressure and 1 kHz. In one embodiment one or more composition comprising one or more RNA substance and one or more RNA stabilizing substance may have a dielectric constant between about 2-20 at 20° C. under standard atmospheric pressure and 1 kHz. In one embodiment one or more composition comprising one or more RNA substance and one or more RNA stabilizing substance may have a dielectric constant between about 3-20 at 20° C. under standard atmospheric pressure and 1 kHz. In one embodiment one or more composition comprising one or more RNA substance and one or more RNA stabilizing substance may have a dielectric constant between about 4-20 at 20° C. under standard atmospheric pressure and 1 kHz. In one embodiment one or more composition comprising one or more RNA substance and one or more RNA stabilizing substance may have a dielectric constant between about 5-20 at 20° C. under standard atmospheric pressure and 1 kHz. In one embodiment one or more composition comprising one or more RNA substance and one or more RNA stabilizing substance may have a dielectric constant between about 10-20 at 20° C. under standard atmospheric pressure and 1 kHz. In one embodiment one or more composition comprising one or more RNA substance and one or more RNA stabilizing substance may have a dielectric constant between about 1-15 at 20° C. under standard atmospheric pressure and 1 kHz. In one embodiment one or more composition comprising one or more RNA substance and one or more RNA stabilizing substance may have a dielectric constant between about 2-15 at 20° C. under standard atmospheric pressure and 1 kHz. In one embodiment one or more composition comprising one or more RNA substance and one or more RNA stabilizing substance may have a dielectric constant between about 3-15 at 20° C. under standard atmospheric pressure and 1 kHz. In one embodiment one or more composition comprising one or more RNA substance and one or more RNA stabilizing substance may have a dielectric constant between about 4-15 at 20° C. under standard atmospheric pressure and 1 kHz. In one embodiment one or more composition comprising one or more RNA substance and one or more RNA stabilizing substance may have a dielectric constant between about 5-15 at 20° C. under standard atmospheric pressure and 1 kHz. In one embodiment one or more composition comprising one or more RNA substance and one or more RNA stabilizing substance may have a dielectric constant between about 1-10 at 20° C. under standard atmospheric pressure and 1 kHz. In one embodiment one or more composition comprising one or more RNA substance and one or more RNA stabilizing substance may have a dielectric constant between about 2-10 at 20° C. under standard atmospheric pressure and 1 kHz. In one embodiment one or more composition comprising one or more RNA substance and one or more RNA stabilizing substance may have a dielectric constant between about 3-10 at 20° C. under standard atmospheric pressure and 1 kHz. In one embodiment one or more composition comprising one or more RNA substance and one or more RNA stabilizing substance may have a dielectric constant between about 4-10 at 20° C. under standard atmospheric pressure and 1 kHz. In one embodiment one or more composition comprising one or more RNA substance and one or more RNA stabilizing substance may have a dielectric constant between about 5-10 at 20° C. under standard atmospheric pressure and 1 kHz.

In one embodiment one or more composition comprising one or more RNA substance and one or more RNA stabilizing substance may comprise one or more water concentrations in the water concentration list or in the water concentration range list. In one embodiment one or more composition comprising one or more RNA substance, one or more RNA stabilizing substance, and one or more additional substance, such as one or more cellular uptake agent as a non-limiting example, may comprise one or more water concentrations in the water concentration list or in the water concentration range list. In one embodiment one or more composition comprising one or more RNA substance and one or more RNA stabilizing substance may have a dielectric constant of one or more dielectric constant in the dielectric constant list or in the dielectric constant range list. In one embodiment one or more composition comprising one or more RNA substance, one or more RNA stabilizing substance, and one or more additional substance, such as one or more cellular uptake agent as a non-limiting example, may have a dielectric constant of one or more dielectric constant in the dielectric constant list or in the dielectric constant range list.

In one embodiment one or more composition comprising one or more RNA substance and one or more low dielectric substance may comprise one or more water concentrations in the water concentration list or in the water concentration range list. In one embodiment one or more composition comprising one or more RNA substance, one or more low dielectric substance, and one or more additional substance, such as one or more cellular uptake agent or one or more additional RNA stabilizing substance as non-limiting examples, may comprise one or more water concentrations in the water concentration list or in the water concentration range list. In one embodiment one or more composition comprising one or more RNA substance and one or more low dielectric substance may have a dielectric constant of one or more dielectric constant in the dielectric constant list or in the dielectric constant range list. In one embodiment one or more composition comprising one or more RNA substance, one or more low dielectric substance, and one or more additional substance, such as one or more cellular uptake agent or one or more additional RNA stabilizing substance as non-limiting examples, may have a dielectric constant of one or more dielectric constant in the dielectric constant list or in the dielectric constant range list.

Embodiments of the present invention may include one or more compositions comprising one or more RNA substance and one or more RNA stabilizing substance, wherein a composition is not lyophilized.

In one embodiment one or more composition comprising one or more RNA substance and one or more RNA stabilizing substance may not be lyophilized. In one embodiment one or more composition comprising one or more RNA substance, one or more RNA stabilizing substance, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more RNA stabilizing substance, wherein a composition may be lyophilized.

In one embodiment one or more composition comprising one or more RNA substance and one or more RNA stabilizing substance may be lyophilized. In one embodiment one or more composition comprising one or more RNA substance, one or more RNA stabilizing substance, and one or more additional substance, such as one or more cellular uptake agent or one or more additional RNA stabilizing substance as non-limiting examples, may be lyophilized.

Embodiments of the present invention may include compositions comprising one or more low dielectric substance used in a composition comprising one or more RNA substance, one or more low dielectric substance, and one or more of the following substances: water, cellular uptake agents, aprotic substances, cyclic phosphate or metaphosphate containing substances, non-carbohydrate organic osmolyte substances, tertiary sulfonium containing substances, quaternary ammonium containing substances, quaternary phosphonium containing substances, hydrotrope containing substances, surfactant containing substances, betaine containing substances, stabilizing monomers, stabilizing polymers, or supplemental RNA stabilizing substances.

Other embodiments may include one or more composition comprising one or more low dielectric substance and one or more RNA substance and may also comprise one or more cellular uptake agent or one or more additional RNA stabilizing substances. Other embodiments may include compositions comprising one or more low dielectric substance, one or more RNA substance, and one or more additional substances, such as: one or more cellular uptake agent, or one or more additional RNA stabilizing substance, or one or more buffering agent, or one or more chelating agent, or one or more inorganic salt, or water, as non-limiting examples.

Embodiments of the present invention may include one or more compositions comprising one or more RNA substance and one or more low dielectric substance, wherein a composition is not lyophilized.

In one embodiment one or more composition comprising one or more RNA substance and one or more low dielectric substance may not be lyophilized. In one embodiment one or more composition comprising one or more RNA substance, one or more low dielectric substance, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more low dielectric substance, wherein a composition may be lyophilized.

In one embodiment one or more composition comprising one or more RNA substance and one or more low dielectric substance may be lyophilized. In one embodiment one or more composition comprising one or more RNA substance, one or more low dielectric substance, and one or more additional substance, such as one or more cellular uptake agent or one or more additional RNA stabilizing substance as non-limiting examples, may be lyophilized.

Embodiments of the present invention may include one or more compositions comprising one or more low dielectric substance and one or more RNA substance described herein, wherein one or more composition described herein may have a melting of one or more melting point in the stabilizing composition melting point list.

In one embodiment one or more composition comprising one or more RNA substance and one or more low dielectric substance may have a melting point of one or more melting point in the stabilizing composition melting point list. In one embodiment one or more composition comprising one or more RNA substance, one or more low dielectric substance, and one or more additional substance, such as one or more cellular uptake agent or one or more additional RNA stabilizing substance as a non-limiting example, may have a melting point of one or more melting point in the stabilizing composition melting point list.

### Cyclic Phosphate or Metaphosphate Containing Substances:

The inventors have discovered that cyclic phosphate or metaphosphate containing substances may stabilize RNA substances.

The inventors have discovered that RNA stabilizing substances may comprise cyclic phosphate or metaphosphate containing substances. The inventors have also discovered that the stability of RNA substances may be enhanced in an RNA storage environment comprising a cyclic phosphate or metaphosphate containing substance. The inventors have also discovered that the stability of RNA substances may be enhanced in an RNA storage environment comprising a cyclic phosphate or metaphosphate containing substance and one or more additional RNA stabilizing substance, such as an aprotic substance.

The inventors have also discovered that the stability of RNA substances may be enhanced in compositions comprising an RNA substance and a cyclic phosphate or metaphosphate containing substance. The inventors have also discovered that the stability of RNA substances may be enhanced in compositions comprising an RNA substance, a cyclic phosphate or metaphosphate containing substance, and one or more additional RNA stabilizing substance, such as an aprotic substance.

In one embodiment of the present invention, one or more RNA stabilizing substance may comprise a cyclic phosphate or metaphosphate containing substance.

One embodiment of the present invention may include a composition comprised of one or more RNA substance and one or more cyclic phosphate or metaphosphate containing substance. Another embodiment of the present invention may include a composition comprised of one or more RNA substance, one or more cyclic phosphate or metaphosphate containing substance, and one or more additional RNA stabilizing substance, such as an aprotic substance.

One embodiment of the present invention may include a combination or mixture comprising one or more RNA substance and one or more cyclic phosphate or metaphosphate containing substance. Another embodiment of the present invention may include a combination or mixture comprising one or more RNA substance, one or more cyclic phosphate or metaphosphate containing substance, and one or more additional RNA stabilizing substance, such as an aprotic substance.

Embodiments of the present invention that comprise one or more RNA substance and one or more cyclic phosphate or metaphosphate containing substance may include combining, such as by mixing, one or more RNA substance with one or more substance that comprises at least one or more cyclic phosphate or metaphosphate containing substance.

Embodiments of the present invention that comprise one or more RNA substance, one or more cyclic phosphate or metaphosphate containing substance, and one or more additional RNA stabilizing substance, such as an aprotic substance, may include combining, such as by mixing, one or more RNA substance with one or more substance that comprises at least one or more cyclic phosphate or metaphosphate containing substance and one or more substance that comprises at least one or more RNA stabilizing substance, such as an aprotic substance.

An embodiment of the present invention may include compositions of materials that comprise one or more RNA substance and at least one or more cyclic phosphate or metaphosphate containing substance. The storage environment that improves the stability of RNA substances may comprise at least one or more vapor, liquid, powder, or solid cyclic phosphate or metaphosphate containing substance.

An embodiment of the present invention may include compositions of materials that comprise one or more RNA substance, one or more cyclic phosphate or metaphosphate containing substance, and one or more additional RNA stabilizing substance, such as an aprotic substance. The storage environment that improves the stability of RNA substances may comprise at least one or more vapor, liquid, powder, or solid cyclic phosphate or metaphosphate containing substance and may comprise at least one or more vapor, liquid, powder, or solid RNA stabilizing substance, such as an aprotic substance.

An embodiment of the present invention may include combinations of materials that comprise one or more RNA substance and at least one or more cyclic phosphate or metaphosphate containing substance. The storage environment that improves the stability of RNA substances may comprise at least one or more vapor, liquid, powder, or solid cyclic phosphate or metaphosphate containing substance.

An embodiment of the present invention may include combinations of materials that comprise one or more RNA substance, one or more cyclic phosphate or metaphosphate containing substance, and one or more additional RNA stabilizing substance, such as an aprotic substance. The storage environment that improves the stability of RNA substances may comprise at least one or more vapor, liquid, powder, or solid cyclic phosphate or metaphosphate containing substance and may comprise at least one or more vapor, liquid, powder, or solid RNA stabilizing substance, such as an aprotic substance.

In one embodiment of the present invention a composition comprised of one or more RNA substance and one or more cyclic phosphate or metaphosphate containing substance, produces a mixture with at least one or more RNA substance and at least one or more cyclic phosphate or metaphosphate containing substance. In one embodiment of the present invention a composition comprised of one or more RNA substance, one or more cyclic phosphate or metaphosphate containing substance, and one or more additional RNA stabilizing substance, such as an aprotic substance, produces a mixture with at least one or more RNA substance, at least one or more cyclic phosphate or metaphosphate containing substance, and at least one or more additional RNA stabilizing substance, such as an aprotic substance.

In one embodiment of the present invention a combination comprising one or more RNA substance and one or more cyclic phosphate or metaphosphate containing substance, produces a mixture with at least one or more RNA substance and at least one or more cyclic phosphate or metaphosphate containing substance. In one embodiment of the present invention a combination comprising one or more RNA substance, one or more cyclic phosphate or metaphosphate containing substance, and one or more additional RNA stabilizing substance, such as an aprotic substance, produces a mixture with at least one or more RNA substance, at least one or more cyclic phosphate or metaphosphate containing substance, and at least one or more additional RNA stabilizing substance, such as an aprotic substance.

In one embodiment of the present invention a composition with improved RNA stability may comprise one or more RNA substance and one or more cyclic phosphate or metaphosphate containing substance. In one embodiment of the present invention a composition with improved RNA stability may comprise one or more RNA substance, one or more cyclic phosphate or metaphosphate containing substance, and one or more additional RNA stabilizing substance, such as an aprotic substance.

In one embodiment of the present invention a composition with improved RNA stability may comprise a mixture of one or more RNA substance and one or more cyclic phosphate or metaphosphate containing substance. In one embodiment of the present invention a composition with improved RNA stability may comprise a mixture of one or more RNA substance, one or more cyclic phosphate or metaphosphate containing substance, and one or more additional RNA stabilizing substance, such as an aprotic substance.

In one embodiment of the present invention, each component included in a composition comprising one or more RNA substance and one or more cyclic phosphate or metaphosphate containing substance, may be stored separately, such as in a kit, or such as individual substances or as mixtures of one or more substance, and then combined later to produce a composition comprising one or more RNA substance and one or more cyclic phosphate or metaphosphate containing substance.

In one embodiment of the present invention, each component included in a composition comprising one or more RNA substance, one or more cyclic phosphate or metaphosphate containing substance, and one or more additional RNA stabilizing substance, such as an aprotic substance, may be stored separately, such as in a kit, or such as individually or as mixtures of one or more substance, and then combined later to produce a composition comprising one or more RNA substance, one or more cyclic phosphate or metaphosphate containing substance, and one or more additional RNA stabilizing substance, such as an aprotic substance.

In one embodiment of the present invention a composition comprising one or more RNA substance and one or more cyclic phosphate or metaphosphate containing substance may be at least partially biocompatible.

In one embodiment of the present invention a composition comprising one or more RNA substance, one or more cyclic phosphate or metaphosphate containing substance, and one or more additional RNA stabilizing substance, such as an aprotic substance, may be at least partially biocompatible.

In one embodiment of the present invention a combination comprised of one or more RNA substance and one or more cyclic phosphate or metaphosphate containing substance may be at least partially biocompatible.

In one embodiment of the present invention a combination comprised of one or more RNA substance, one or more cyclic phosphate or metaphosphate containing substance, and one or more additional RNA stabilizing substance, such as an aprotic substance, may be at least partially biocompatible.

Embodiments of the present invention comprising at least one or more cyclic phosphate or metaphosphate containing substances may include one or more forms of the substance. These forms may include, but are not limited to, one more counterions, ionic forms, conjugate bases, conjugate acids, protonated or deprotonated forms, hydrated or dehydrated forms, isomers, structural isomers, stereo isomers, chiral forms, salts, or combinations thereof.

In one embodiment a cyclic phosphate or metaphosphate containing substance may comprise one or more of the following formulas: [PO₃]ₘ, [HPO₃]ₘ, [NaPO₃]ₘ, or [KPO₃]ₘ, wherein m is an integer and m may be between about 2-6, or between about 2-10, or between about 2-20, or between about 2-50, or between about 2-100, or between about 2-500, or between about 2-1,000, or between about 2-10,000, or between about 2-100,000. In one embodiment a cyclic phosphate or metaphosphate containing substance may comprise one or more of the following formulas: [PO₃], [HPO₃]ₘ, [NaPO₃]ₘ, or [KPO₃]ₘ, wherein m is an integer and m may be between about 3-6, or between about 3-10, or between about 3-20, or between about 3-50, or between about 3-100, or between about 3-500, or between about 3-1,000, or between about 3-10,000, or between about 3-100,000. In one embodiment a cyclic phosphate or metaphosphate containing substance may comprise one or more of the following formulas: [PO₃], [HPO₃]ₘ, [NaPO₃]ₘ, or [KPO₃]ₘ, wherein m is an integer and m may be between about 4-6, or between about 4-10, or between about 4-20, or between about 4-50, or between about 4-100, or between about 4-500, or between about 4-1,000, or between about 4-10,000, or between about 4-100,000. In one embodiment a cyclic phosphate or metaphosphate containing substance may comprise one or more of the following formulas: [PO₃]ₘ, [HPO₃]ₘ, [NaPO₃]ₘ, or [KPO₃]ₘ, wherein m is an integer and m may be between about 6-10, or between about 6-20, or between about 6-50, or between about 6-100, or between about 6-500, or between about 6-1,000, or between about 6-10,000, or between about 6-100,000. In one embodiment a cyclic phosphate or metaphosphate containing substance may comprise one or more of the following formulas: [PO₃]ₘ, [HPO₃]ₘ, [NaPO₃]ₘ, or [KPO₃]ₘ, wherein m is an integer and m may be 3, 4, 5, 6, 7, 8, 9, 10, 11, 12, 13, 14, 15, 16, 17, 18, 19, or 20, or greater than 20.

In one embodiment a cyclic phosphate or metaphosphate containing substance may be anionic. In one embodiment a cyclic phosphate or metaphosphate containing substance may comprise an anion. In one embodiment a cyclic phosphate or metaphosphate containing substance may be neutrally charged.

In one embodiment one or more counterion, such as H⁺, Na⁺ or K⁺, as non-limiting examples, may be associated with one or more cyclic phosphate or metaphosphate containing substance to balance one or more charges. In one embodiment one or more counterion, such as Na⁺ or K⁺, as non-limiting examples, may be substituted for one or more H⁺ ion. In one embodiment one or more counterion such as Na⁺ or K⁺, as non-limiting examples, may be substituted for one or more or even substantially all H⁺ ions. In one embodiment one or more counterions, such as H⁺, Na⁺, or K⁺, as non-limiting examples, may be substituted for one or more other counterions such as H⁺, Na⁺, or K⁺, as non-limiting examples.

In one embodiment a cyclic phosphate or metaphosphate containing substance may comprise a heterocyclic ring structure. In one embodiment a cyclic phosphate or metaphosphate containing substance may comprise at least part of a heterocyclic ring structure. In one embodiment a cyclic phosphate or metaphosphate containing substance may comprise an ultraphosphate ring structure. In one embodiment a cyclic phosphate or metaphosphate containing substance may comprise at least part of an ultraphosphate ring structure.

A non-limiting example of a cyclic phosphate or metaphosphate containing substance that may be used is hexametaphosphate (HMP), such as sodium hexametaphosphate as a non-limiting example, wherein HMP may be substituted for or used in combination with one or more cyclic phosphate or metaphosphate containing substance as described herein. One embodiment of the present invention may be a combination of substances comprising HMP and another embodiment may also comprise at least one RNA substance. Other embodiments may be combinations of substances comprising HMP wherein the concentration of HMP may be one or more concentration in the stabilizing substance weight percent concentration list or in the stabilizing substance molar concentration range list or in the stabilizing substance molar concentration list.

A non-limiting example of a cyclic phosphate or metaphosphate containing substance that may be used is trimetaphosphate (TMP), such as sodium trimetaphosphate as a non-limiting example, wherein TMP may be substituted for or used in combination with one or more cyclic phosphate or metaphosphate containing substance as described herein. One embodiment of the present invention may be a combination of substances comprising TMP and another embodiment may also comprise at least one RNA substance. Other embodiments may be combinations of substances comprising TMP wherein the concentration of TMP may be one or more concentration in the stabilizing substance weight percent concentration list or in the stabilizing substance molar concentration range list or in the stabilizing substance molar concentration list.

A non-limiting example of a cyclic phosphate or metaphosphate containing substance that may be used is tetrametaphosphate, such as sodium tetrametaphosphate as a non-limiting example, wherein tetrametaphosphate may be substituted for or used in combination with one or more cyclic phosphate or metaphosphate containing substance as described herein. One embodiment of the present invention may be a combination of substances comprising tetrametaphosphate and another embodiment may also comprise at least one RNA substance. Other embodiments may be combinations of substances comprising tetrametaphosphate wherein the concentration of tetrametaphosphate may be one or more concentration in the stabilizing substance weight percent concentration list or in the stabilizing substance molar concentration range list or in the stabilizing substance molar concentration list.

A non-limiting example of a cyclic phosphate or metaphosphate containing substance that may be used is cyclic-2,3-diphosphoglycerate, wherein cyclic-2,3-diphosphoglycerate may be substituted for or used in combination with one or more cyclic phosphate or metaphosphate containing substance as described herein. One embodiment of the present invention may be a combination of substances comprising cyclic-2,3-diphosphoglycerate and another embodiment may also comprise at least one RNA substance. Other embodiments may be combinations of substances comprising cyclic-2,3-diphosphoglycerate wherein the concentration of cyclic-2,3-diphosphoglycerate may be one or more concentration in the stabilizing substance weight percent concentration list or in the stabilizing substance molar concentration range list or in the stabilizing substance molar concentration list.

For conciseness, the following list of cyclic phosphate or metaphosphate containing substances is herein referred to as the cyclic phosphate or metaphosphate containing substance list wherein one or more of the following substances may be substituted for or used in combination with one or more cyclic phosphate or metaphosphate containing substance as described herein.

In other embodiments, other cyclic phosphate or metaphosphate containing substances that may be substituted for or used in combination with one or more cyclic phosphate or metaphosphate containing substance as described herein may comprise one or more following, including but not limited to: hexametaphosphate, sodium hexametaphosphate, potassium hexametaphosphate, tetrametaphosphate, sodium tetrametaphosphate, potassium tetrametaphosphate, trimetaphosphate, sodium trimetaphosphate, potassium trimetaphosphate, cyclic-2,3-diphosphoglycerate, 1,5-μ-oxo-tetrametaphosphate, fused-ring ultraphosphates, cyclic ultraphosphates, cyclic phosphates, metaphosphates, or derivatives or combinations thereof.

Embodiments of the present invention may include combinations comprising one or more substance from the cyclic phosphate or metaphosphate containing substance list substituted for or used in combination with one or more cyclic phosphate or metaphosphate containing substance or one or more RNA stabilizing substance, such as a betaine containing substance as a non-limiting example, and other embodiments may also comprise at least one RNA substance.

Other embodiments may include combinations of substances comprising one or more cyclic phosphate or metaphosphate containing substance selected from the cyclic phosphate or metaphosphate containing substance list wherein the concentration of one or more substances may be one or more concentration in the stabilizing substance weight percent concentration list or in the stabilizing substance molar concentration range list or in the stabilizing substance molar concentration list.

In one embodiment, a cyclic phosphate or metaphosphate containing substance may have a molecular weight between about 100-100,000 daltons. In one embodiment, a cyclic phosphate or metaphosphate containing substance may have a molecular weight between about 100-50,000 daltons. In one embodiment, a cyclic phosphate or metaphosphate containing substance may have a molecular weight between about 100-20,000 daltons. In one embodiment, a cyclic phosphate or metaphosphate containing substance may have a molecular weight between about 100-10,000 daltons. In one embodiment, a cyclic phosphate or metaphosphate containing substance may have a molecular weight between about 100-5,000 daltons. In one embodiment, a cyclic phosphate or metaphosphate containing substance may have a molecular weight between about 100-2,000 daltons. In one embodiment, a cyclic phosphate or metaphosphate containing substance may have a molecular weight between about 100-1,000 daltons. In one embodiment, a cyclic phosphate or metaphosphate containing substance may have a molecular weight between about 100-900 daltons. In one embodiment, a cyclic phosphate or metaphosphate containing substance may have a molecular weight between about 100-800 daltons. In one embodiment, a cyclic phosphate or metaphosphate containing substance may have a molecular weight between about 100-700 daltons. In one embodiment, a cyclic phosphate or metaphosphate containing substance may have a molecular weight between about 100-600 daltons. In one embodiment, a cyclic phosphate or metaphosphate containing substance may have a molecular weight between about 100-500 daltons.

In one embodiment, a cyclic phosphate or metaphosphate containing substance may have a molecular weight between about 200-100,000 daltons. In one embodiment, a cyclic phosphate or metaphosphate containing substance may have a molecular weight between about 200-50,000 daltons. In one embodiment, a cyclic phosphate or metaphosphate containing substance may have a molecular weight between about 200-20,000 daltons. In one embodiment, a cyclic phosphate or metaphosphate containing substance may have a molecular weight between about 200-10,000 daltons. In one embodiment, a cyclic phosphate or metaphosphate containing substance may have a molecular weight between about 200-5,000 daltons. In one embodiment, a cyclic phosphate or metaphosphate containing substance may have a molecular weight between about 200-2,000 daltons. In one embodiment, a cyclic phosphate or metaphosphate containing substance may have a molecular weight between about 200-1,000 daltons. In one embodiment, a cyclic phosphate or metaphosphate containing substance may have a molecular weight between about 200-900 daltons. In one embodiment, a cyclic phosphate or metaphosphate containing substance may have a molecular weight between about 200-800 daltons. In one embodiment, a cyclic phosphate or metaphosphate containing substance may have a molecular weight between about 200-700 daltons. In one embodiment, a cyclic phosphate or metaphosphate containing substance may have a molecular weight between about 200-600 daltons. In one embodiment, a cyclic phosphate or metaphosphate containing substance may have a molecular weight between about 200-500 daltons.

One embodiment of the present invention is the method whereby one or more cyclic phosphate or metaphosphate containing substance may be combined, such as by mixing, with at least one or more RNA substance to produce a mixture comprising at least one or more cyclic phosphate or metaphosphate containing substance and at least one or more RNA substance. As a non-limiting example one or more RNA substance may be mixed with one or more cyclic phosphate or metaphosphate containing substance.

Another embodiment of the present invention is the method whereby one or more cyclic phosphate or metaphosphate containing substance may be combined, such as by mixing, with at least one or more RNA substance to produce a mixture comprising at least one or more RNA substance and at least one or more cyclic phosphate or metaphosphate containing substance at one or more of the RNA stabilizing substance concentrations within the stabilizing substance molar concentration range list or in the stabilizing substance molar concentration list or stabilizing substance weight percent concentration list as described herein. These same methods may be used to combine one or more other substances, including, but not limited to one or more cellular uptake agents or one or more additional RNA stabilizing substances, with one or more RNA substances and one or more cyclic phosphate or metaphosphate containing substances to produce a mixture comprising one or more RNA substance, one or more cyclic phosphate or metaphosphate containing substance, and one or more cellular uptake agent or one or more additional RNA stabilizing substance.

One embodiment of the present invention is the method whereby one or more cyclic phosphate or metaphosphate containing substance may be combined, such as by mixing, with at least one or more RNA substance to produce a composition comprising at least one or more RNA substance and at least one or more cyclic phosphate or metaphosphate containing substance. Another embodiment of the present invention is the method whereby one or more cyclic phosphate or metaphosphate containing substance may be combined, such as by mixing, with one or more RNA substance to produce a composition comprising at least one or more RNA substance and at least one or more cyclic phosphate or metaphosphate containing substance at one or more of the RNA stabilizing substance concentrations within the stabilizing substance molar concentration range list or in the stabilizing substance molar concentration list or stabilizing substance weight percent concentration list as described herein. These same methods may be used to combine one or more other substances, including, but not limited to one or more cellular uptake agents or one or more additional RNA stabilizing substance, with one or more RNA substance and one or more cyclic phosphate or metaphosphate containing substance to produce a composition comprising one or more RNA substance, one or more cyclic phosphate or metaphosphate containing substance, and one or more cellular uptake agent or one or more additional RNA stabilizing substance.

In one embodiment a cyclic phosphate or metaphosphate containing substance may comprise one or more anionic moiety at about physiologic pH.

In one embodiment a cyclic phosphate or metaphosphate containing substance may comprise one or more PIF as described herein.

In one embodiment, one or more cyclic phosphate or metaphosphate containing substance may be at least part of a polymer comprised of at least one or more cyclic phosphate or metaphosphate containing substances.

In one embodiment one or more composition comprising one or more RNA substance and one or more cyclic phosphate or metaphosphate containing substance may comprise one or more water concentrations in the water concentration list or in the water concentration range list. In one embodiment one or more composition comprising one or more RNA substance, one or more cyclic phosphate or metaphosphate containing substance, and one or more additional substance, such as one or more cellular uptake agent or one or more additional RNA stabilizing substance as non-limiting examples, may comprise one or more water concentrations in the water concentration list or in the water concentration range list. In one embodiment one or more composition comprising one or more RNA substance and one or more cyclic phosphate or metaphosphate containing substance may have a dielectric constant of one or more dielectric constant in the dielectric constant list or in the dielectric constant range list. In one embodiment one or more composition comprising one or more RNA substance, one or more cyclic phosphate or metaphosphate containing substance, and one or more additional substance, such as one or more cellular uptake agent or one or more additional RNA stabilizing substance as non-limiting examples, may have a dielectric constant of one or more dielectric constant in the dielectric constant list or in the dielectric constant range list.

Embodiments of the present invention may include compositions comprising one or more cyclic phosphate or metaphosphate containing substance used in a composition comprising one or more RNA substance, one or more cyclic phosphate or metaphosphate containing substance, and one or more of the following substances: water, cellular uptake agents, aprotic substances, low dielectric substances, non-carbohydrate organic osmolyte substances, tertiary sulfonium containing substances, quaternary ammonium containing substances, quaternary phosphonium containing substances, hydrotrope containing substances, surfactant containing substances, betaine containing substances, stabilizing monomers, stabilizing polymers, or supplemental RNA stabilizing substances. Other embodiments may include one or more composition comprising one or more cyclic phosphate or metaphosphate containing substance and one or more RNA substance and may also comprise one or more cellular uptake agent or one or more additional RNA stabilizing substances. Other embodiments may include compositions comprising one or more cyclic phosphate or metaphosphate containing substance, one or more RNA substance, and one or more additional substances, such as: one or more cellular uptake agent, or one or more additional RNA stabilizing substance, or one or more buffering agent, or one or more chelating agent, or one or more inorganic salt, or water, as non-limiting examples.

Embodiments of the present invention may include one or more compositions comprising one or more RNA substance and one or more cyclic phosphate or metaphosphate containing substance, wherein a composition is not lyophilized.

In one embodiment one or more composition comprising one or more RNA substance and one or more cyclic phosphate or metaphosphate containing substance may not be lyophilized. In one embodiment one or more composition comprising one or more RNA substance, one or more cyclic phosphate or metaphosphate containing substance, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more cyclic phosphate or metaphosphate containing substance, wherein a composition may be lyophilized.

In one embodiment one or more composition comprising one or more RNA substance and one or more cyclic phosphate or metaphosphate containing substance may be lyophilized. In one embodiment one or more composition comprising one or more RNA substance, one or more cyclic phosphate or metaphosphate containing substance, and one or more additional substance, such as one or more cellular uptake agent or one or more additional RNA stabilizing substance as non-limiting examples, may be lyophilized.

Embodiments of the present invention may include one or more compositions comprising one or more cyclic phosphate or metaphosphate containing substance and one or more RNA substance described herein, wherein one or more composition described herein may have a melting of one or more melting point in the stabilizing composition melting point list.

In one embodiment one or more composition comprising one or more RNA substance and one or more cyclic phosphate or metaphosphate containing substance may have a melting point of one or more melting point in the stabilizing composition melting point list. In one embodiment one or more composition comprising one or more RNA substance, one or more cyclic phosphate or metaphosphate containing substance, and one or more additional substance, such as one or more cellular uptake agent or one or more additional RNA stabilizing substance as a non-limiting example, may have a melting point of one or more melting point in the stabilizing composition melting point list.

### Non-Carbohydrate Organic Osmolyte Substances:

The inventors have discovered that non-carbohydrate organic osmolyte substances may stabilize RNA substances.

The inventors have discovered that RNA stabilizing substances may comprise non-carbohydrate organic osmolyte substances. The inventors have also discovered that the stability of RNA substances may be enhanced in an RNA storage environment comprising a non-carbohydrate organic osmolyte substance. The inventors have also discovered that the stability of RNA substances may be enhanced in an RNA storage environment comprising a non-carbohydrate organic osmolyte substance and one or more additional RNA stabilizing substance, such as an aprotic substance.

The inventors have also discovered that the stability of RNA substances may be enhanced in compositions comprising an RNA substance and a non-carbohydrate organic osmolyte substance. The inventors have also discovered that the stability of RNA substances may be enhanced in compositions comprising an RNA substance, a non-carbohydrate organic osmolyte substance, and one or more additional RNA stabilizing substance, such as an aprotic substance.

In one embodiment of the present invention, one or more RNA stabilizing substance may comprise a non-carbohydrate organic osmolyte substance.

One embodiment of the present invention may include a composition comprised of one or more RNA substance and one or more non-carbohydrate organic osmolyte substance. Another embodiment of the present invention may include a composition comprised of one or more RNA substance, one or more non-carbohydrate organic osmolyte substance, and one or more additional RNA stabilizing substance, such as an aprotic substance.

One embodiment of the present invention may include a combination or mixture comprising one or more RNA substance and one or more non-carbohydrate organic osmolyte substance. Another embodiment of the present invention may include a combination or mixture comprising one or more RNA substance, one or more non-carbohydrate organic osmolyte substance, and one or more additional RNA stabilizing substance, such as an aprotic substance.

Embodiments of the present invention that comprise one or more RNA substance and one or more non-carbohydrate organic osmolyte substance may include combining, such as by mixing, one or more RNA substance with one or more substance that comprises at least one or more non-carbohydrate organic osmolyte substance.

Embodiments of the present invention that comprise one or more RNA substance, one or more non-carbohydrate organic osmolyte substance, and one or more additional RNA stabilizing substance, such as an aprotic substance, may include combining, such as by mixing, one or more RNA substance with one or more substance that comprises at least one or more non-carbohydrate organic osmolyte substance and one or more substance that comprises at least one or more RNA stabilizing substance, such as an aprotic substance.

An embodiment of the present invention may include compositions of materials that comprise one or more RNA substance and at least one or more non-carbohydrate organic osmolyte substance. The storage environment that improves the stability of RNA substances may comprise at least one or more vapor, liquid, powder, or solid non-carbohydrate organic osmolyte substance.

An embodiment of the present invention may include compositions of materials that comprise one or more RNA substance, one or more non-carbohydrate organic osmolyte substance, and one or more additional RNA stabilizing substance, such as an aprotic substance. The storage environment that improves the stability of RNA substances may comprise at least one or more vapor, liquid, powder, or solid non-carbohydrate organic osmolyte substance and may comprise at least one or more vapor, liquid, powder, or solid RNA stabilizing substance, such as an aprotic substance.

An embodiment of the present invention may include combinations of materials that comprise one or more RNA substance and at least one or more non-carbohydrate organic osmolyte substance. The storage environment that improves the stability of RNA substances may comprise at least one or more vapor, liquid, powder, or solid non-carbohydrate organic osmolyte substance.

An embodiment of the present invention may include combinations of materials that comprise one or more RNA substance, one or more non-carbohydrate organic osmolyte substance, and one or more additional RNA stabilizing substance, such as an aprotic substance. The storage environment that improves the stability of RNA substances may comprise at least one or more vapor, liquid, powder, or solid non-carbohydrate organic osmolyte substance and may comprise at least one or more vapor, liquid, powder, or solid RNA stabilizing substance, such as an aprotic substance.

In one embodiment of the present invention a composition comprised of one or more RNA substance and one or more non-carbohydrate organic osmolyte substance, produces a mixture with at least one or more RNA substance and at least one or more non-carbohydrate organic osmolyte substance. In one embodiment of the present invention a composition comprised of one or more RNA substance, one or more non-carbohydrate organic osmolyte substance, and one or more additional RNA stabilizing substance, such as an aprotic substance, produces a mixture with at least one or more RNA substance, at least one or more non-carbohydrate organic osmolyte substance, and at least one or more additional RNA stabilizing substance, such as an aprotic substance.

In one embodiment of the present invention a combination comprising one or more RNA substance and one or more non-carbohydrate organic osmolyte substance, produces a mixture with at least one or more RNA substance and at least one or more non-carbohydrate organic osmolyte substance. In one embodiment of the present invention a combination comprising one or more RNA substance, one or more non-carbohydrate organic osmolyte substance, and one or more additional RNA stabilizing substance, such as an aprotic substance, produces a mixture with at least one or more RNA substance, at least one or more non-carbohydrate organic osmolyte substance, and at least one or more additional RNA stabilizing substance, such as an aprotic substance.

In one embodiment of the present invention a composition with improved RNA stability may comprise one or more RNA substance and one or more non-carbohydrate organic osmolyte substance. In one embodiment of the present invention a composition with improved RNA stability may comprise one or more RNA substance, one or more non-carbohydrate organic osmolyte substance, and one or more additional RNA stabilizing substance, such as an aprotic substance.

In one embodiment of the present invention a composition with improved RNA stability may comprise a mixture of one or more RNA substance and one or more non-carbohydrate organic osmolyte substance. In one embodiment of the present invention a composition with improved RNA stability may comprise a mixture of one or more RNA substance, one or more non-carbohydrate organic osmolyte substance, and one or more additional RNA stabilizing substance, such as an aprotic substance.

In one embodiment of the present invention, each component included in a composition comprising one or more RNA substance and one or more non-carbohydrate organic osmolyte substance, may be stored separately, such as in a kit, or such as individual substances or as mixtures of one or more substance, and then combined later to produce a composition comprising one or more RNA substance and one or more non-carbohydrate organic osmolyte substance.

In one embodiment of the present invention, each component included in a composition comprising one or more RNA substance, one or more non-carbohydrate organic osmolyte substance, and one or more additional RNA stabilizing substance, such as an aprotic substance, may be stored separately, such as in a kit, or such as individually or as mixtures of one or more substance, and then combined later to produce a composition comprising one or more RNA substance, one or more non-carbohydrate organic osmolyte substance, and one or more additional RNA stabilizing substance, such as an aprotic substance.

In one embodiment of the present invention a composition comprising one or more RNA substance and one or more non-carbohydrate organic osmolyte substance may be at least partially biocompatible.

In one embodiment of the present invention a composition comprising one or more RNA substance, one or more non-carbohydrate organic osmolyte substance, and one or more additional RNA stabilizing substance, such as an aprotic substance, may be at least partially biocompatible.

In one embodiment of the present invention a combination comprised of one or more RNA substance and one or more non-carbohydrate organic osmolyte substance may be at least partially biocompatible.

In one embodiment of the present invention a combination comprised of one or more RNA substance, one or more non-carbohydrate organic osmolyte substance, and one or more additional RNA stabilizing substance, such as an aprotic substance, may be at least partially biocompatible.

Embodiments of the present invention comprising at least one or more non-carbohydrate organic osmolyte substances may include one or more forms of the substance. These forms may include, but are not limited to, one more counterions, ionic forms, conjugate bases, conjugate acids, protonated or deprotonated forms, hydrated or dehydrated forms, isomers, structural isomers, stereo isomers, chiral forms, salts, or combinations thereof.

In one embodiment a non-carbohydrate organic osmolyte substance may comprise one or more amino acid, such as glycine or proline, or beta-alanine as non-limiting examples. In one embodiment a non-carbohydrate organic osmolyte substance may comprise one or more amino acid derivative, such as ectoine or taurine as non-limiting examples. In one embodiment a non-carbohydrate organic osmolyte substance may comprise one or more beta-amino acid, such as beta-alanine as a non-limiting example. In one embodiment a non-carbohydrate organic osmolyte substance may comprise one or more non-proteinogenic amino acid, such as pipecolic acid or gamma-aminobutyric acid as non-limiting examples. In one embodiment a non-carbohydrate organic osmolyte substance may comprise one or more cyclic amino acid, or one or more cyclic amino acid derivative, or one or more cyclic beta amino acid, or one or more cyclic non-proteinogenic amino acid, such as proline, ectoine, or pipecolic acid as a non-limiting example.

In one embodiment, one or more amino acid may comprise one or more amino acid derivative, or one or more beta-amino acid, or one or more non-proteinogenic amino acid, or one or more cyclic amino acid, or one or more cyclic amino acid derivative, or one or more cyclic beta amino acid, or one or more cyclic non-proteinogenic amino acid.

In one embodiment a non-carbohydrate organic osmolyte substance may comprise one or more amino acid, wherein one or more amino acid, or amino acid derivative, or beta-amino acid, or non-proteinogenic amino acid may be hydroxylated. In one embodiment a non-carbohydrate organic osmolyte substance may comprise one or more amino acid, wherein one or more amino acid, or amino acid derivative, or beta-amino acid, or non-proteinogenic amino acid may comprise 1 or more hydroxy groups, 2 or more hydroxy groups, 3 or more hydroxy groups, 4 or more hydroxy groups, or 5 or more hydroxy groups. In one embodiment a non-carbohydrate organic osmolyte substance may comprise one or more amino acid, wherein one or more amino acid, or amino acid derivative, or beta-amino acid, or non-proteinogenic amino acid may comprise between about 1-10 hydroxy groups, or between about 1-5 hydroxy groups, or between about 1-3 hydroxy groups, or between about 2-10 hydroxy groups, or between about 2-5 hydroxy groups, or between about 2-3 hydroxy groups.

In one embodiment a non-carbohydrate organic osmolyte substance may comprise one or more amino acid, wherein one or more amino acid, or amino acid derivative, or beta-amino acid, or non-proteinogenic amino acid may be carboxylated. In one embodiment a non-carbohydrate organic osmolyte substance may comprise one or more amino acid, wherein one or more amino acid, or amino acid derivative, or beta-amino acid, or non-proteinogenic amino acid may comprise 1 or more carboxylate or carboxylic acid groups, 2 or more carboxylate or carboxylic acid groups, 3 or more carboxylate or carboxylic acid groups, 4 or more carboxylate or carboxylic acid groups, or 5 or more carboxylate or carboxylic acid groups. In one embodiment a non-carbohydrate organic osmolyte substance may comprise one or more amino acid, wherein one or more amino acid, or amino acid derivative, or beta-amino acid, or non-proteinogenic amino acid may comprise between about 1-10 carboxylate or carboxylic acid groups, or between about 1-5 carboxylate or carboxylic acid groups, or between about 1-3 carboxylate or carboxylic acid groups, or between about 2-10 carboxylate or carboxylic acid groups, or between about 2-5 carboxylate or carboxylic acid groups, or between about 2-3 carboxylate or carboxylic acid groups.

In one embodiment a non-carbohydrate organic osmolyte substance may not comprise an amino acid. In one embodiment a non-carbohydrate organic osmolyte substance may not comprise an amino acid derivative. In one embodiment a non-carbohydrate organic osmolyte substance may not comprise a beta-amino acid. In one embodiment a non-carbohydrate organic osmolyte substance may not comprise a non-proteinogenic amino acid. In one embodiment a non-carbohydrate organic osmolyte substance may not comprise a cyclic amino acid, or a cyclic amino acid derivative, or a cyclic beta-amino acid, or a cyclic non-proteinogenic amino acid. In one embodiment a non-carbohydrate organic osmolyte substance may not comprise a peptide. In one embodiment a non-carbohydrate organic osmolyte substance may not comprise a polypeptide.

As used herein a peptide is a chain of 4 or more consecutive amino acids linked by amide bonds.

In one embodiment a non-carbohydrate organic osmolyte substance may comprise one or more low molecular weight aliphatic polyamine, such as spermidine, spermine, or putrescine as non-limiting examples.

In one embodiment a low molecular weight aliphatic polyamine may have a molecular weight less than 1,000 daltons. In one embodiment a low molecular weight aliphatic polyamine may have a molecular weight less than 800 daltons. In one embodiment a low molecular weight aliphatic polyamine may have a molecular weight less than 600 daltons. In one embodiment a low molecular weight aliphatic polyamine may have a molecular weight less than 500 daltons. In one embodiment a low molecular weight aliphatic polyamine may have a molecular weight between about 50-1,000 daltons. In one embodiment a low molecular weight aliphatic polyamine may have a molecular weight between about 50-800 daltons. In one embodiment a low molecular weight aliphatic polyamine may have a molecular weight between about 50-600 daltons. In one embodiment a low molecular weight aliphatic polyamine may have a molecular weight between about 50-500 daltons.

In one embodiment a low molecular weight aliphatic polyamine may comprise 2 or more nitrogen atoms. In one embodiment a low molecular weight aliphatic polyamine may comprise 3 or more nitrogen atoms. In one embodiment a low molecular weight aliphatic polyamine may comprise 4 or more nitrogen atoms. In one embodiment a low molecular weight aliphatic polyamine may comprise between about 2-10 nitrogen atoms. In one embodiment a low molecular weight aliphatic polyamine may comprise between about 2-8 nitrogen atoms. In one embodiment a low molecular weight aliphatic polyamine may comprise between about 2-6 nitrogen atoms. In one embodiment a low molecular weight aliphatic polyamine may comprise between about 2-5 nitrogen atoms. In one embodiment a low molecular weight aliphatic polyamine may comprise between about 3-10 nitrogen atoms. In one embodiment a low molecular weight aliphatic polyamine may comprise between about 3-8 nitrogen atoms. In one embodiment a low molecular weight aliphatic polyamine may comprise between about 3-6 nitrogen atoms. In one embodiment a low molecular weight aliphatic polyamine may comprise between about 3-5 nitrogen atoms. In one embodiment a low molecular weight aliphatic polyamine may comprise between about 4-10 nitrogen atoms. In one embodiment a low molecular weight aliphatic polyamine may comprise between about 4-8 nitrogen atoms. In one embodiment a low molecular weight aliphatic polyamine may comprise between about 4-6 nitrogen atoms.

In one embodiment a non-carbohydrate organic osmolyte substance may comprise one or more low molecular weight aliphatic polyamine, wherein one or more low molecular weight aliphatic polyamine may be hydroxylated. In one embodiment a non-carbohydrate organic osmolyte substance may comprise one or more low molecular weight aliphatic polyamine, wherein one or more low molecular weight aliphatic polyamine may comprise 1 or more hydroxy groups, 2 or more hydroxy groups, 3 or more hydroxy groups, 4 or more hydroxy groups, or 5 or more hydroxy groups. In one embodiment a non-carbohydrate organic osmolyte substance may comprise one or more low molecular weight aliphatic polyamine, wherein one or more low molecular weight aliphatic polyamine may comprise between about 1-10 hydroxy groups, or between about 1-5 hydroxy groups, or between about 1-3 hydroxy groups, or between about 2-10 hydroxy groups, or between about 2-5 hydroxy groups, or between about 2-3 hydroxy groups.

In one embodiment a non-carbohydrate organic osmolyte substance may comprise one or more low molecular weight aliphatic polyamine, wherein one or more low molecular weight aliphatic polyamine may be carboxylated. In one embodiment a non-carbohydrate organic osmolyte substance may comprise one or more low molecular weight aliphatic polyamine, wherein one or more low molecular weight aliphatic polyamine may comprise 1 or more carboxylate or carboxylic acid groups, 2 or more carboxylate or carboxylic acid groups, 3 or more carboxylate or carboxylic acid groups, 4 or more carboxylate or carboxylic acid groups, or 5 or more carboxylate or carboxylic acid groups. In one embodiment a non-carbohydrate organic osmolyte substance may comprise one or more low molecular weight aliphatic polyamine, wherein one or more low molecular weight aliphatic polyamine may comprise between about 1-10 carboxylate or carboxylic acid groups, or between about 1-5 carboxylate or carboxylic acid groups, or between about 1-3 carboxylate or carboxylic acid groups, or between about 2-10 carboxylate or carboxylic acid groups, or between about 2-5 carboxylate or carboxylic acid groups, or between about 2-3 carboxylate or carboxylic acid groups.

A non-limiting example of a non-carbohydrate organic osmolyte substance comprised of an amino acid that may be used is glycine, wherein glycine may be substituted for or used in combination with one or more non-carbohydrate organic osmolyte substances described herein. One embodiment of the present invention may be a combination of substances comprising glycine and another embodiment may also comprise at least one RNA substance. Other embodiments may be combinations of substances comprising glycine wherein the concentration of glycine may be one or more concentration in the stabilizing substance weight percent concentration list or in the stabilizing substance molar concentration range list or in the stabilizing substance molar concentration list.

A non-limiting example of a non-carbohydrate organic osmolyte substance comprised of an amino acid that may be used is proline, wherein proline may be substituted for or used in combination with one or more non-carbohydrate organic osmolyte substances described herein. One embodiment of the present invention may be a combination of substances comprising proline and another embodiment may also comprise at least one RNA substance. Other embodiments may be combinations of substances comprising proline wherein the concentration of proline may be one or more concentration in the stabilizing substance weight percent concentration list or in the stabilizing substance molar concentration range list or in the stabilizing substance molar concentration list.

A non-limiting example of a non-carbohydrate organic osmolyte substance comprised of an amino acid derivative that may be used is ectoine, wherein ectoine may be substituted for or used in combination with one or more non-carbohydrate organic osmolyte substances described herein. One embodiment of the present invention may be a combination of substances comprising ectoine and another embodiment may also comprise at least one RNA substance. Other embodiments may be combinations of substances comprising ectoine wherein the concentration of ectoine may be one or more concentration in the stabilizing substance weight percent concentration list or in the stabilizing substance molar concentration range list or in the stabilizing substance molar concentration list.

A non-limiting example of a non-carbohydrate organic osmolyte substance comprised of an amino acid derivative that may be used is hydroxy ectoine, wherein hydroxy ectoine may be substituted for or used in combination with one or more non-carbohydrate organic osmolyte substances described herein. One embodiment of the present invention may be a combination of substances comprising hydroxy ectoine and another embodiment may also comprise at least one RNA substance. Other embodiments may be combinations of substances comprising hydroxy ectoine wherein the concentration of hydroxy ectoine may be one or more concentration in the stabilizing substance weight percent concentration list or in the stabilizing substance molar concentration range list or in the stabilizing substance molar concentration list.

A non-limiting example of a non-carbohydrate organic osmolyte substance comprised of an amino acid derivative that may be used is taurine, wherein taurine may be substituted for or used in combination with one or more non-carbohydrate organic osmolyte substances described herein. One embodiment of the present invention may be a combination of substances comprising taurine and another embodiment may also comprise at least one RNA substance. Other embodiments may be combinations of substances comprising taurine wherein the concentration of taurine may be one or more concentration in the stabilizing substance weight percent concentration list or in the stabilizing substance molar concentration range list or in the stabilizing substance molar concentration list.

A non-limiting example of a non-carbohydrate organic osmolyte substance comprised of a beta-amino acid that may be used is beta-alanine, wherein beta-alanine may be substituted for or used in combination with one or more non-carbohydrate organic osmolyte substances described herein. One embodiment of the present invention may be a combination of substances comprising beta-alanine and another embodiment may also comprise at least one RNA substance. Other embodiments may be combinations of substances comprising beta-alanine wherein the concentration of beta-alanine may be one or more concentration in the stabilizing substance weight percent concentration list or in the stabilizing substance molar concentration range list or in the stabilizing substance molar concentration list.

A non-limiting example of a non-carbohydrate organic osmolyte substance comprised of a non-proteinogenic amino acid that may be used is pipecolic acid, wherein pipecolic acid may be substituted for or used in combination with one or more non-carbohydrate organic osmolyte substances described herein. One embodiment of the present invention may be a combination of substances comprising pipecolic acid and another embodiment may also comprise at least one RNA substance. Other embodiments may be combinations of substances comprising pipecolic acid wherein the concentration of pipecolic acid may be one or more concentration in the stabilizing substance weight percent concentration list or in the stabilizing substance molar concentration range list or in the stabilizing substance molar concentration list.

A non-limiting example of a non-carbohydrate organic osmolyte substance comprised of a low molecular weight aliphatic polyamine that may be used is spermidine, wherein spermidine may be substituted for or used in combination with one or more non-carbohydrate organic osmolyte substances described herein. One embodiment of the present invention may be a combination of substances comprising spermidine and another embodiment may also comprise at least one RNA substance. Other embodiments may be combinations of substances comprising spermidine wherein the concentration of spermidine may be one or more concentration in the stabilizing substance weight percent concentration list or in the stabilizing substance molar concentration range list or in the stabilizing substance molar concentration list.

A non-limiting example of a non-carbohydrate organic osmolyte substance comprised of a low molecular weight aliphatic polyamine that may be used is spermine, wherein spermine may be substituted for or used in combination with one or more non-carbohydrate organic osmolyte substances described herein. One embodiment of the present invention may be a combination of substances comprising spermine and another embodiment may also comprise at least one RNA substance. Other embodiments may be combinations of substances comprising spermine wherein the concentration of spermine may be one or more concentration in the stabilizing substance weight percent concentration list or in the stabilizing substance molar concentration range list or in the stabilizing substance molar concentration list.

For conciseness the following list of non-limiting example non-carbohydrate organic osmolyte substances comprised of one or more amino acid is herein referred to as the osmolyte amino acid list. Other non-limiting examples of other non-carbohydrate organic osmolyte substances comprised of one or more amino acid that may be used, may include, but are not limited to, one or more of the following: alanine, asparagine, aspartate, cysteine, glutamine, glycine, isoleucine, lysine, methionine, proline, serine, threonine, or valine, or derivatives, or combinations thereof.

For conciseness the following list of non-limiting example non-carbohydrate organic osmolyte substances comprised of one or more amino acid is herein referred to as the osmolyte amino acid derivative list. Other non-limiting examples of other non-carbohydrate organic osmolyte substances comprised of one or more amino acid derivative that may be used, may include, but are not limited to, one or more of the following: ectoine, taurine, hydroxyectoine, N-acetylcysteine, N-acetylglutamate, aspartame, methionine sulfoxide, cystine, hydroxy proline, oxoproline, acetylhydroxyproline, alanyl-glutamine, glutathione, carboxyglutamic acid, methyllysine, allysine, hydroxylysine, pyroglutamic acid, cystathionine, lanthionine, djenkolic acid, diaminopimelic acid, or pantothenic acid, or derivatives, or combinations thereof.

For conciseness the following list of non-limiting example non-carbohydrate organic osmolyte substances comprised of one or more beta-amino acid is herein referred to as the osmolyte beta-amino acid list. Other non-limiting examples of other non-carbohydrate organic osmolyte substances comprised of one or more beta-amino acid that may be used, may include, but are not limited to, one or more of the following: beta-alanine, beta-alanine, beta-leucine, beta-lysine, beta-glutamate, or beta-glutamine, or derivatives, or combinations thereof.

For conciseness the following list of non-limiting example non-carbohydrate organic osmolyte substances comprised of one or more non-proteinogenic amino acid is herein referred to as the osmolyte non-proteinogenic amino acid list. Other non-limiting examples of other non-carbohydrate organic osmolyte substances comprised of one or more non-proteinogenic amino acid that may be used, may include, but are not limited to, one or more of the following: pipecolic acid, gamma-aminobutyric acid, citrulline, aminolevulinic acid, aminocyclopropane carboxylic acid, homocysteine, aminobutyric acid, canavanine, methylalanine, methylvaline, dehydroalanine, aminoisobutyric acid, norvaline, norleucine, or ornithine, or derivatives, or combinations thereof.

For conciseness the following list of non-limiting example non-carbohydrate organic osmolyte substances comprised of one or more low molecular weight aliphatic polyamine is herein referred to as the osmolyte low molecular weight aliphatic polyamine list. Other non-limiting examples of other non-carbohydrate organic osmolyte substances comprised of one or more low molecular weight aliphatic polyamine that may be used, may include, but are not limited to, one or more of the following: urea, cadaverine, putrescine, diaminopropane,1-3-diaminopropane, spermidine, spermine, thermospermine, norspermidine, norspermine, homocaldopentamine, hypusine, (aminopropyl)-cadaverine, N¹-(aminopropyl)-cadaverine, N¹,N′-bis(aminopropyl)-cadaverine, caldopentamine, caldohexamine, tris(3-aminopropyl)amine, N-acetylputrescine, N-acetylspermine, N¹-acetylspermidine, N¹-acetylspermine, dimethylaminopropylamine, or derivatives, or combinations thereof.

For conciseness, the following list of non-carbohydrate organic osmolyte substances, is herein referred to as the non-carbohydrate organic osmolyte substance list wherein one or more of the following substances may be substituted for or used in combination with one or more non-carbohydrate organic osmolyte substance as described herein.

In other embodiments, other non-carbohydrate organic osmolyte substances that may be substituted for or used in combination with one or more non-carbohydrate organic osmolyte substance as described herein may comprise one or more of the following, including but not limited to: one or more substances in the osmolyte amino acid list, one or more substances in the osmolyte amino acid derivative list, one or more substances in the osmolyte beta-amino acid list, one or more substances in the osmolyte non-proteinogenic amino acid list, one or more substances in the osmolyte low molecular weight aliphatic polyamine list, N-methylpipecolic acid, glutathione, 4-hydroxyproline, 5-hydroxylysine, homoserine, homocysteine, beta-alanine, statine, pyrrolysine, hydroxylysine, selenocysteine, gamma-carboxybutric acid, spermidic acid, aceglutamide, N-acetylaspartic acid, acetylcysteine, N-acetylcysteine, acetylleucine, sarcosine, N-methylglycine, N,N-dimethylglycine, dimethylglycine, N6-acetyl-lysine, ((R)-1-{6-[(R)-2-carboxypyrrolidin-1-yl]-6-oxohexanoyl}pyrrolidine-2-carboxylic acid) (CPHPC), dimethylalanine, formiminoglutamic acid, N-formylmethionine, glutaurine, 4-(gamma-glutamylamino)butanoic acid, beta-Hydroxy beta-methylbutyric acid, beta-hydroxy beta-methylbutyrate, 3-hydroxyasparagine, lanthionine ketimine, 3,4-dihydro-2H-1,3-thiazine-3,5-dicarboxylic acid, methionine sulfoxide, N-methyl-L-glutamic acid, S-methylcysteine, 3-methylhistidine, N-methylornithine, milacemide, opines, nopaline, octopine, or agropine, or salts, or derivatives, or combinations thereof.

Embodiments of the present invention may include combinations comprising one or more of the substances from the non-carbohydrate organic osmolyte substance list substituted for or used in combination with one or more non-carbohydrate organic osmolyte substance or one or more RNA stabilizing substance, such as an aprotic substance as a non-limiting example, and other embodiments may also comprise at least one RNA substance.

Other embodiments may include combinations of substances comprising one or more non-carbohydrate organic osmolyte substance selected from the non-carbohydrate organic osmolyte substance list wherein the concentration of one or more substances may be one or more concentration in the stabilizing substance weight percent concentration list or in the stabilizing substance molar concentration range list or in the stabilizing substance molar concentration list.

In one embodiment, a non-carbohydrate organic osmolyte substance may have a molecular weight between about 50-5,000 daltons. In one embodiment, a non-carbohydrate organic osmolyte substance may have a molecular weight between about 50-2,000 daltons. In one embodiment, a non-carbohydrate organic osmolyte substance may have a molecular weight between about 50-1,000 daltons. In one embodiment, a non-carbohydrate organic osmolyte substance may have a molecular weight between about 50-900 daltons. In one embodiment, a non-carbohydrate organic osmolyte substance may have a molecular weight between about 50-800 daltons. In one embodiment, a non-carbohydrate organic osmolyte substance may have a molecular weight between about 50-700 daltons. In one embodiment, a non-carbohydrate organic osmolyte substance may have a molecular weight between about 50-600 daltons. In one embodiment, a non-carbohydrate organic osmolyte substance may have a molecular weight between about 50-500 daltons. In one embodiment, a non-carbohydrate organic osmolyte substance may have a molecular weight between about 50-400 daltons. In one embodiment, a non-carbohydrate organic osmolyte substance may have a molecular weight between about 50-300 daltons. In one embodiment, a non-carbohydrate organic osmolyte substance may have a molecular weight between about 50-250 daltons.

In one embodiment, a non-carbohydrate organic osmolyte substance may have a molecular weight between about 100-5,000 daltons. In one embodiment, a non-carbohydrate organic osmolyte substance may have a molecular weight between about 100-2,000 daltons. In one embodiment, a non-carbohydrate organic osmolyte substance may have a molecular weight between about 100-1,000 daltons. In one embodiment, a non-carbohydrate organic osmolyte substance may have a molecular weight between about 100-900 daltons. In one embodiment, a non-carbohydrate organic osmolyte substance may have a molecular weight between about 100-800 daltons. In one embodiment, a non-carbohydrate organic osmolyte substance may have a molecular weight between about 100-700 daltons. In one embodiment, a non-carbohydrate organic osmolyte substance may have a molecular weight between about 100-600 daltons. In one embodiment, a non-carbohydrate organic osmolyte substance may have a molecular weight between about 100-500 daltons. In one embodiment, a non-carbohydrate organic osmolyte substance may have a molecular weight between about 100-400 daltons. In one embodiment, a non-carbohydrate organic osmolyte substance may have a molecular weight between about 100-300 daltons. In one embodiment, a non-carbohydrate organic osmolyte substance may have a molecular weight between about 100-250 daltons.

One embodiment of the present invention is the method whereby one or more non-carbohydrate organic osmolyte substance may be combined, such as by mixing, with at least one or more RNA substance to produce a mixture comprising at least one or more non-carbohydrate organic osmolyte substance and at least one or more RNA substance. As a non-limiting example one or more RNA substance may be mixed with one or more non-carbohydrate organic osmolyte substance.

Another embodiment of the present invention is the method whereby one or more non-carbohydrate organic osmolyte substance may be combined, such as by mixing, with at least one or more RNA substance to produce a mixture comprising at least one or more RNA substance and at least one or more non-carbohydrate organic osmolyte substance at one or more of the RNA stabilizing substance concentrations within the stabilizing substance molar concentration range list or in the stabilizing substance molar concentration list or the stabilizing substance weight percent concentration list as described herein. These same methods may be used to combine one or more other substances, including, but not limited to one or more cellular uptake agents or one or more additional RNA stabilizing substances, with one or more RNA substances and one or more non-carbohydrate organic osmolyte substances to produce a mixture comprising one or more RNA substance, one or more non-carbohydrate organic osmolyte substance, and one or more cellular uptake agent or one or more additional RNA stabilizing substance.

One embodiment of the present invention is the method whereby one or more non-carbohydrate organic osmolyte substance may be combined, such as by mixing, with at least one or more RNA substance to produce a composition comprising at least one or more RNA substance and at least one or more non-carbohydrate organic osmolyte substance. Another embodiment of the present invention is the method whereby one or more non-carbohydrate organic osmolyte substance may be combined, such as by mixing, with one or more RNA substance to produce a composition comprising at least one or more RNA substance and at least one or more non-carbohydrate organic osmolyte substance at one or more of the RNA stabilizing substance concentrations within the stabilizing substance molar concentration range list or in the stabilizing substance molar concentration list or stabilizing substance weight percent concentration list as described herein. These same methods may be used to combine one or more other substances, including, but not limited to one or more cellular uptake agents or one or more additional RNA stabilizing substance, with one or more RNA substance and one or more non-carbohydrate organic osmolyte substance to produce a composition comprising one or more RNA substance, one or more non-carbohydrate organic osmolyte substance, and one or more cellular uptake agent or one or more additional RNA stabilizing substance.

In one embodiment a non-carbohydrate organic osmolyte substance may be aliphatic.

In one embodiment a non-carbohydrate organic osmolyte substance may comprise a ring structure. In another embodiment a non-carbohydrate organic osmolyte substance may comprise a heterocyclic ring structure. In one embodiment a non-carbohydrate organic osmolyte substance may comprise an aromatic ring structure. In another embodiment a non-carbohydrate organic osmolyte substance may comprise an aromatic heterocyclic ring structure.

In one embodiment a non-carbohydrate organic osmolyte may not comprise an aromatic ring structure. In one embodiment a non-carbohydrate organic osmolyte may not comprise a heterocyclic aromatic ring structure. In one embodiment a non-carbohydrate organic osmolyte may not comprise a quaternary ammonium. In one embodiment a non-carbohydrate organic osmolyte may not comprise a tertiary sulfonium. In one embodiment a non-carbohydrate organic osmolyte may not comprise a betaine.

In one embodiment a non-carbohydrate organic osmolyte substance may comprise a cationic moiety at about physiologic pH. In one embodiment a non-carbohydrate organic osmolyte substance may comprise an anionic moiety at about physiologic pH. In one embodiment non-carbohydrate organic osmolyte substance may comprise a zwitterion at about physiologic pH.

In one embodiment a non-carbohydrate organic osmolyte substance may comprise one or more PIF as described herein.

In one embodiment, one or more non-carbohydrate organic osmolyte substance may be at least part of a polymer comprised of at least one or more non-carbohydrate organic osmolyte substances.

In one embodiment one or more composition comprising one or more RNA substance and one or more non-carbohydrate organic osmolyte substance may comprise one or more water concentrations in the water concentration list or in the water concentration range list. In one embodiment one or more composition comprising one or more RNA substance, one or more non-carbohydrate organic osmolyte substance, and one or more additional substance, such as one or more cellular uptake agent or one or more additional RNA stabilizing substance as non-limiting examples, may comprise one or more water concentrations in the water concentration list or in the water concentration range list. In one embodiment one or more composition comprising one or more RNA substance and one or more non-carbohydrate organic osmolyte substance may have a dielectric constant of one or more dielectric constant in the dielectric constant list or in the dielectric constant range list. In one embodiment one or more composition comprising one or more RNA substance, one or more non-carbohydrate organic osmolyte substance, and one or more additional substance, such as one or more cellular uptake agent or one or more additional RNA stabilizing substance as non-limiting examples, may have a dielectric constant of one or more dielectric constant in the dielectric constant list or in the dielectric constant range list.

Embodiments of the present invention may include compositions comprising one or more non-carbohydrate organic osmolyte substance used in a composition comprising one or more RNA substance, one or more non-carbohydrate organic osmolyte substance, and one or more of the following substances: water, cellular uptake agents, aprotic substances, low dielectric substances, cyclic phosphate or metaphosphate containing substances, tertiary sulfonium containing substances, quaternary ammonium containing substances, quaternary phosphonium containing substances, hydrotrope containing substances, surfactant containing substances, betaine containing substances, stabilizing monomers, stabilizing polymers, or supplemental RNA stabilizing substances. Other embodiments may include one or more composition comprising one or more non-carbohydrate organic osmolyte substance and one or more RNA substance and may also comprise one or more cellular uptake agent or one or more additional RNA stabilizing substances. Other embodiments may include compositions comprising one or more non-carbohydrate organic osmolyte substance, one or more RNA substance, and one or more additional substances, such as: one or more cellular uptake agent, or one or more additional RNA stabilizing substance, or one or more buffering agent, or one or more chelating agent, or one or more inorganic salt, or water, as non-limiting examples.

Embodiments of the present invention may include one or more compositions comprising one or more RNA substance and one or more non-carbohydrate organic osmolyte substance, wherein a composition is not lyophilized.

In one embodiment one or more composition comprising one or more RNA substance and one or more non-carbohydrate organic osmolyte substance may not be lyophilized. In one embodiment one or more composition comprising one or more RNA substance, one or more non-carbohydrate organic osmolyte substance, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more non-carbohydrate organic osmolyte substance, wherein a composition may be lyophilized.

In one embodiment one or more composition comprising one or more RNA substance and one or more non-carbohydrate organic osmolyte substance may be lyophilized. In one embodiment one or more composition comprising one or more RNA substance, one or more non-carbohydrate organic osmolyte substance, and one or more additional substance, such as one or more cellular uptake agent or one or more additional RNA stabilizing substance as non-limiting examples, may be lyophilized.

Embodiments of the present invention may include one or more compositions comprising one or more non-carbohydrate organic osmolyte substance and one or more RNA substance described herein, wherein one or more composition described herein may have a melting of one or more melting point in the stabilizing composition melting point list.

In one embodiment one or more composition comprising one or more RNA substance and one or more non-carbohydrate organic osmolyte substance may have a melting point of one or more melting point in the stabilizing composition melting point list. In one embodiment one or more composition comprising one or more RNA substance, one or more non-carbohydrate organic osmolyte substance, and one or more additional substance, such as one or more cellular uptake agent or one or more additional RNA stabilizing substance as a non-limiting example, may have a melting point of one or more melting point in the stabilizing composition melting point list.

### Tertiary Sulfonium Substances:

The inventors have discovered that tertiary sulfonium containing substances may stabilize RNA substances.

The inventors have discovered that RNA stabilizing substances may comprise tertiary sulfonium containing substances. The inventors have also discovered that the stability of RNA substances may be enhanced in an RNA storage environment comprising a tertiary sulfonium containing substance. The inventors have also discovered that the stability of RNA substances may be enhanced in an RNA storage environment comprising a tertiary sulfonium containing substance and one or more additional RNA stabilizing substance, such as an aprotic substance.

The inventors have also discovered that the stability of RNA substances may be enhanced in compositions comprising an RNA substance and a tertiary sulfonium containing substance. The inventors have also discovered that the stability of RNA substances may be enhanced in compositions comprising an RNA substance, a tertiary sulfonium containing substance, and one or more additional RNA stabilizing substance, such as an aprotic substance.

In one embodiment of the present invention, one or more RNA stabilizing substance may comprise a tertiary sulfonium containing substance.

One embodiment of the present invention may include a composition comprised of one or more RNA substance and one or more tertiary sulfonium containing substance. Another embodiment of the present invention may include a composition comprised of one or more RNA substance, one or more tertiary sulfonium containing substance, and one or more additional RNA stabilizing substance, such as an aprotic substance.

One embodiment of the present invention may include a combination or mixture comprising one or more RNA substance and one or more tertiary sulfonium containing substance. Another embodiment of the present invention may include a combination or mixture comprising one or more RNA substance, one or more tertiary sulfonium containing substance, and one or more additional RNA stabilizing substance, such as an aprotic substance.

Embodiments of the present invention that comprise one or more RNA substance and one or more tertiary sulfonium containing substance may include combining, such as by mixing, one or more RNA substance with one or more substance that comprises at least one or more tertiary sulfonium containing substance.

Embodiments of the present invention that comprise one or more RNA substance, one or more tertiary sulfonium containing substance, and one or more additional RNA stabilizing substance, such as an aprotic substance, may include combining, such as by mixing, one or more RNA substance with one or more substance that comprises at least one or more tertiary sulfonium containing substance and one or more substance that comprises at least one or more RNA stabilizing substance, such as an aprotic substance.

An embodiment of the present invention may include compositions of materials that comprise one or more RNA substance and at least one or more tertiary sulfonium containing substance. The storage environment that improves the stability of RNA substances may comprise at least one or more vapor, liquid, powder, or solid tertiary sulfonium containing substance.

An embodiment of the present invention may include compositions of materials that comprise one or more RNA substance, one or more tertiary sulfonium containing substance, and one or more additional RNA stabilizing substance, such as an aprotic substance. The storage environment that improves the stability of RNA substances may comprise at least one or more vapor, liquid, powder, or solid tertiary sulfonium containing substance and may comprise at least one or more vapor, liquid, powder, or solid RNA stabilizing substance, such as an aprotic substance.

An embodiment of the present invention may include combinations of materials that comprise one or more RNA substance and at least one or more tertiary sulfonium containing substance. The storage environment that improves the stability of RNA substances may comprise at least one or more vapor, liquid, powder, or solid tertiary sulfonium containing substance.

An embodiment of the present invention may include combinations of materials that comprise one or more RNA substance, one or more tertiary sulfonium containing substance, and one or more additional RNA stabilizing substance, such as an aprotic substance. The storage environment that improves the stability of RNA substances may comprise at least one or more vapor, liquid, powder, or solid tertiary sulfonium containing substance and may comprise at least one or more vapor, liquid, powder, or solid RNA stabilizing substance, such as an aprotic substance.

In one embodiment of the present invention a composition comprised of one or more RNA substance and one or more tertiary sulfonium containing substance, produces a mixture with at least one or more RNA substance and at least one or more tertiary sulfonium containing substance.

In one embodiment of the present invention a composition comprised of one or more RNA substance, one or more tertiary sulfonium containing substance, and one or more additional RNA stabilizing substance, such as an aprotic substance, produces a mixture with at least one or more RNA substance, at least one or more tertiary sulfonium containing substance, and at least one or more additional RNA stabilizing substance, such as an aprotic substance.

In one embodiment of the present invention a combination comprising one or more RNA substance and one or more tertiary sulfonium containing substance, produces a mixture with at least one or more RNA substance and at least one or more tertiary sulfonium containing substance. In one embodiment of the present invention a combination comprising one or more RNA substance, one or more tertiary sulfonium containing substance, and one or more additional RNA stabilizing substance, such as an aprotic substance, produces a mixture with at least one or more RNA substance, at least one or more tertiary sulfonium containing substance, and at least one or more additional RNA stabilizing substance, such as an aprotic substance.

In one embodiment of the present invention a composition with improved RNA stability may comprise one or more RNA substance and one or more tertiary sulfonium containing substance. In one embodiment of the present invention a composition with improved RNA stability may comprise one or more RNA substance, one or more tertiary sulfonium containing substance, and one or more additional RNA stabilizing substance, such as an aprotic substance.

In one embodiment of the present invention a composition with improved RNA stability may comprise a mixture of one or more RNA substance and one or more tertiary sulfonium containing substance. In one embodiment of the present invention a composition with improved RNA stability may comprise a mixture of one or more RNA substance, one or more tertiary sulfonium containing substance, and one or more additional RNA stabilizing substance, such as an aprotic substance.

In one embodiment of the present invention, each component included in a composition comprising one or more RNA substance and one or more tertiary sulfonium containing substance, may be stored separately, such as in a kit, or such as individual substances or as mixtures of one or more substance, and then combined later to produce a composition comprising one or more RNA substance and one or more tertiary sulfonium containing substance.

In one embodiment of the present invention, each component included in a composition comprising one or more RNA substance, one or more tertiary sulfonium containing substance, and one or more additional RNA stabilizing substance, such as an aprotic substance, may be stored separately, such as in a kit, or such as individually or as mixtures of one or more substance, and then combined later to produce a composition comprising one or more RNA substance, one or more tertiary sulfonium containing substance, and one or more additional RNA stabilizing substance, such as an aprotic substance.

In one embodiment of the present invention a composition comprising one or more RNA substance and one or more tertiary sulfonium containing substance may be at least partially biocompatible.

In one embodiment of the present invention a composition comprising one or more RNA substance, one or more tertiary sulfonium containing substance, and one or more additional RNA stabilizing substance, such as an aprotic substance, may be at least partially biocompatible.

In one embodiment of the present invention a combination comprised of one or more RNA substance and one or more tertiary sulfonium containing substance may be at least partially biocompatible.

In one embodiment of the present invention a combination comprised of one or more RNA substance, one or more tertiary sulfonium containing substance, and one or more additional RNA stabilizing substance, such as an aprotic substance, may be at least partially biocompatible.

Embodiments of the present invention comprising at least one or more tertiary sulfonium containing substances may include one or more forms of the substance. These forms may include, but are not limited to, one more counterions, ionic forms, conjugate bases, conjugate acids, protonated or deprotonated forms, hydrated or dehydrated forms, isomers, structural isomers, stereo isomers, chiral forms, salts, or combinations thereof.

In one embodiment a tertiary sulfonium containing substance may comprise one or more tertiary sulfonium cation. In one embodiment a tertiary sulfonium containing substance may comprise 1 or more tertiary sulfonium cations, 2 or more tertiary sulfonium cations, 3 or more tertiary sulfonium cations, 4 or more tertiary sulfonium cations, 5 or more tertiary sulfonium cations, 10 or more tertiary sulfonium cations, 50 or more tertiary sulfonium cations, or 100 or more tertiary sulfonium cations. In one embodiment a tertiary sulfonium containing substance may comprise between about 1-1,000 tertiary sulfonium cations, or between about 1-500 tertiary sulfonium cations, or between about 1-200 tertiary sulfonium cations, or between about 1-100 tertiary sulfonium cations, or between about 1-50 tertiary sulfonium cations, or between about 1-tertiary sulfonium cations, or between about 1-10 tertiary sulfonium cations, or between about 1-5 tertiary sulfonium cations, or between about 1-3 tertiary sulfonium cations, or between about 1-2 tertiary sulfonium cations, or between about 2-1,000 tertiary sulfonium cations, or between about 2-500 tertiary sulfonium cations, or between about 2-200 tertiary sulfonium cations, or between about 2-100 tertiary sulfonium cations, or between about 2-50 tertiary sulfonium cations, or between about 2-20 tertiary sulfonium cations, or between about 2-10 tertiary sulfonium cations, or between about 2-5 tertiary sulfonium cations, or between about 2-3 tertiary sulfonium cations.

In one embodiment a tertiary sulfonium containing substance may be hydroxylated. In one embodiment a tertiary sulfonium containing substance may comprise one or more hydroxy groups. In one embodiment a tertiary sulfonium containing substance may comprise 1 or more hydroxy groups, 2 or more hydroxy groups, 3 or more hydroxy groups, 4 or more hydroxy groups, 5 or more hydroxy groups, 10 or more hydroxy groups, or 50 or more hydroxy groups. In one embodiment a tertiary sulfonium containing substance may comprise between about 1-100 hydroxy groups, or between about 1-50 hydroxy groups, or between about 1-20 hydroxy groups, or between about 1-10 hydroxy groups, or between about 1-5 hydroxy groups, or between about 1-3 hydroxy groups, or between about 2-100 hydroxy groups, or between about 2-50 hydroxy groups, or between about 2-20 hydroxy groups, or between about 2-10 hydroxy groups, or between about 2-5 hydroxy groups, or between about 2-3 hydroxy groups.

In one embodiment a tertiary sulfonium containing substance may be carboxylated. In one embodiment a tertiary sulfonium containing substance may comprise one or more carboxylate or carboxylic acid groups. In one embodiment a tertiary sulfonium containing substance may comprise 1 or more carboxylate or carboxylic acid groups, 2 or more carboxylate or carboxylic acid groups, 3 or more carboxylate or carboxylic acid groups, 4 or more carboxylate or carboxylic acid groups, 5 or more carboxylate or carboxylic acid groups, 10 or more carboxylate or carboxylic acid groups, or 50 or more carboxylate or carboxylic acid groups. In one embodiment a tertiary sulfonium containing substance may comprise between about 1-100 carboxylate or carboxylic acid groups, or between about 1-50 carboxylate or carboxylic acid groups, or between about 1-20 carboxylate or carboxylic acid groups, or between about 1-10 carboxylate or carboxylic acid groups, or between about 1-5 carboxylate or carboxylic acid groups, or between about 1-3 carboxylate or carboxylic acid groups, or between about 2-100 carboxylate or carboxylic acid groups, or between about 2-50 carboxylate or carboxylic acid groups, or between about 2-20 carboxylate or carboxylic acid groups, or between about 2-10 carboxylate or carboxylic acid groups, or between about 2-5 carboxylate or carboxylic acid groups, or between about 2-3 carboxylate or carboxylic acid groups.

In one embodiment a tertiary sulfonium containing substance may comprise one or more ester, such as one or more carboxylate ester or carbonate ester as non-limiting examples. In one embodiment a tertiary sulfonium containing substance may comprise 1 or more esters, 2 or more esters, 3 or more esters, 4 or more esters, 5 or more esters, 10 or more esters, or 50 or more esters. In one embodiment a tertiary sulfonium containing substance may comprise between about 1-100 esters, or between about 1-50 esters, or between about 1-20 esters, or between about 1-10 esters, or between about 1-5 esters, or between about 1-3 esters, or between about 2-100 esters, or between about 2-50 esters, or between about 2-20 esters, or between about 2-10 esters, or between about 2-esters, or between about 2-3 esters.

In one embodiment a tertiary sulfonium containing substance may comprise one or more ester bond. In one embodiment a tertiary sulfonium containing substance may comprise 1 or more ester bonds, 2 or more ester bonds, 3 or more ester bonds, 4 or more ester bonds, or 5 or more ester bonds, 10 or more ester bonds, or 50 or more ester bonds. In one embodiment a tertiary sulfonium containing substance may comprise between about 1-100 ester bonds, or between about 1-50 ester bonds, or between about 1-20 ester bonds, or between about 1-10 ester bonds, or between about 1-ester bonds, or between about 1-3 ester bonds, or between about 2-100 ester bonds, or between about 2-50 ester bonds, or between about 2-20 ester bonds, or between about 2-10 ester bonds, or between about 2-5 ester bonds, or between about 2-3 ester bonds.

In one embodiment a tertiary sulfonium containing substance may comprise one or more ether bond. In one embodiment a tertiary sulfonium containing substance may comprise 1 or more ether bonds, 2 or more ether bonds, 3 or more ether bonds, 4 or more ether bonds, or 5 or more ether bonds, 10 or more ether bonds, or 50 or more ether bonds. In one embodiment a tertiary sulfonium containing substance may comprise between about 1-100 ether bonds, or between about 1-50 ether bonds, or between about 1-20 ether bonds, or between about 1-10 ether bonds, or between about 1-5 ether bonds, or between about 1-3 ether bonds, or between about 2-100 ether bonds, or between about 2-50 ether bonds, or between about 2-20 ether bonds, or between about 2-10 ether bonds, or between about 2-5 ether bonds, or between about 2-3 ether bonds.

In one embodiment a tertiary sulfonium containing substance may comprise one or more ether. In one embodiment a tertiary sulfonium containing substance may comprise 1 or more ethers, 2 or more ethers, 3 or more ethers, 4 or more ethers, or 5 or more ethers, 10 or more ethers, or 50 or more ethers. In one embodiment a tertiary sulfonium containing substance may comprise between about 1-100 ethers, or between about 1-50 ethers, or between about 1-20 ethers, or between about 1-10 ethers, or between about 1-5 ethers, or between about 1-3 ethers, or between about 2-100 ethers, or between about 2-50 ethers, or between about 2-20 ethers, or between about 2-10 ethers, or between about 2-5 ethers, or between about 2-3 ethers.

In one embodiment a tertiary sulfonium containing substance may comprise one or more amide bond. In one embodiment a tertiary sulfonium containing substance may comprise 1 or more amide bonds, 2 or more amide bonds, 3 or more amide bonds, 4 or more amide bonds, 5 or more amide bonds, 10 or more amide bonds, or 50 or more amide bonds. In one embodiment a tertiary sulfonium containing substance may comprise between about 1-100 amide bonds, or between about 1-50 amide bonds, or between about 1-20 amide bonds, or between about 1-10 amide bonds, or between about 1-5 amide bonds, or between about 1-3 amide bonds, or between about 2-100 amide bonds, or between about 2-50 amide bonds, or between about 2-20 amide bonds, or between about 2-10 amide bonds, or between about 2-5 amide bonds, or between about 2-3 amide bonds.

In one embodiment a tertiary sulfonium containing substance may comprise one or more amide group. In one embodiment a tertiary sulfonium containing substance may comprise 1 or more amide groups, 2 or more amide groups, 3 or more amide groups, 4 or more amide groups, 5 or more amide groups, or 10 or more amide groups, or 50 or more amide groups. In one embodiment a tertiary sulfonium containing substance may comprise between about 1-100 amide groups, or between about 1-50 amide groups, or between about 1-20 amide groups, or between about 1-10 amide groups, or between about 1-5 amide groups, or between about 1-3 amide groups, or between about 2-100 amide groups, or between about 2-50 amide groups, or between about 2-amide groups, or between about 2-10 amide groups, or between about 2-5 amide groups, or between about 2-3 amide groups.

In one embodiment a tertiary sulfonium containing substance may comprise one or more organosulfate group. In one embodiment a tertiary sulfonium containing substance may comprise 1 or more organosulfate groups, 2 or more organosulfate groups, 3 or more organosulfate groups, 4 or more organosulfate groups, 5 or more organosulfate groups, 10 or more organosulfate groups, or 50 or more organosulfate groups. In one embodiment a tertiary sulfonium containing substance may comprise between about 1-100 organosulfate groups, or between about 1-50 organosulfate groups, or between about 1-20 organosulfate groups, or between about 1-10 organosulfate groups, or between about 1-5 organosulfate groups, or between about 1-3 organosulfate groups, or between about 2-100 organosulfate groups, or between about 2-50 organosulfate groups, or between about 2-20 organosulfate groups, or between about 2-10 organosulfate groups, or between about 2-5 organosulfate groups, or between about 2-3 organosulfate groups.

In one embodiment a tertiary sulfonium containing substance may comprise one or more sulfonate group. In one embodiment a tertiary sulfonium containing substance may comprise 1 or more sulfonate groups, 2 or more sulfonate groups, 3 or more sulfonate groups, 4 or more sulfonate groups, 5 or more sulfonate groups, 10 or more sulfonate groups, or 50 or more sulfonate groups. In one embodiment a tertiary sulfonium containing substance may comprise between about 1-100 sulfonate groups, or between about 1-50 sulfonate groups, or between about 1-20 sulfonate groups, or between about 1-10 sulfonate groups, or between about 1-5 sulfonate groups, or between about 1-3 sulfonate groups, or between about 2-100 sulfonate groups, or between about 2-50 sulfonate groups, or between about 2-20 sulfonate groups, or between about 2-10 sulfonate groups, or between about 2-5 sulfonate groups, or between about 2-3 sulfonate groups.

In one embodiment a tertiary sulfonium containing substance may comprise one or more organophosphate group. In one embodiment a tertiary sulfonium containing substance may comprise 1 or more organophosphate groups, 2 or more organophosphate groups, 3 or more organophosphate groups, 4 or more organophosphate groups, 5 or more organophosphate groups, or more organophosphate groups, or 50 or more organophosphate groups. In one embodiment a tertiary sulfonium containing substance may comprise between about 1-100 organophosphate groups, or between about 1-50 organophosphate groups, or between about 1-20 organophosphate groups, or between about 1-10 organophosphate groups, or between about 1-5 organophosphate groups, or between about 1-3 organophosphate groups, or between about 2-100 organophosphate groups, or between about 2-50 organophosphate groups, or between about 2-20 organophosphate groups, or between about 2-10 organophosphate groups, or between about 2-5 organophosphate groups, or between about 2-3 organophosphate groups.

In one embodiment a tertiary sulfonium containing substance may not comprise a hydroxy group. In one embodiment a tertiary sulfonium containing substance may not comprise a carboxylate or carboxylic acid group. In one embodiment a tertiary sulfonium containing substance may not comprise an ester bond. In one embodiment a tertiary sulfonium containing substance may not comprise an ester, such as a carboxylate ester or carbonate ester as non-limiting examples. In one embodiment a tertiary sulfonium containing substance may not comprise an ether. In one embodiment a tertiary sulfonium containing substance may not comprise an ether bond. In one embodiment a tertiary sulfonium containing substance may not comprise an organosulfate group. In one embodiment a tertiary sulfonium containing substance may not comprise a sulfonate group. In one embodiment a tertiary sulfonium containing substance may not comprise an organophosphate group.

In one embodiment a tertiary sulfonium containing substance may comprise one or more polymer. In one embodiment a tertiary sulfonium containing substance may be polymeric. In one embodiment a tertiary sulfonium containing substance may comprise one or more cation. In one embodiment a tertiary sulfonium containing substance may be cationic. In one embodiment a tertiary sulfonium containing substance may be polycationic. In one embodiment a tertiary sulfonium containing substance may comprise one or more anion. In one embodiment a tertiary sulfonium containing substance may comprise one or more zwitterion. In one embodiment a tertiary sulfonium containing substance may be zwitterionic.

In one embodiment a tertiary sulfonium containing substance may comprise one or more ring structure. In one embodiment a tertiary sulfonium containing substance may comprise one or more heterocyclic ring structure. In one embodiment a tertiary sulfonium containing substance may comprise one or more five membered ring structure. In one embodiment a tertiary sulfonium containing substance may comprise one or more six membered ring structure. In one embodiment a tertiary sulfonium containing substance may comprise one or more heterocyclic six membered ring structure. In one embodiment a tertiary sulfonium containing substance may comprise one or more heterocyclic five membered ring structure.

In one embodiment a tertiary sulfonium containing substance may comprise one or more tertiary sulfonium cation that is at least part of a heterocyclic ring structure. In one embodiment a tertiary sulfonium containing substance may comprise one or more tertiary sulfonium cation that is at least part of a five membered heterocyclic ring structure. In one embodiment a tertiary sulfonium containing substance may comprise one or more tertiary sulfonium cation that is at least part of a six membered heterocyclic ring structure.

In one embodiment a tertiary sulfonium containing substance may not be polymeric. In one embodiment a tertiary sulfonium containing substance may not be polycationic. In one embodiment a tertiary sulfonium containing substance may not comprise an anion. In one embodiment a tertiary sulfonium containing substance may not comprise a zwitterion. In one embodiment a tertiary sulfonium containing substance may not be zwitterionic.

In one embodiment a tertiary sulfonium containing substance may not comprise a ring structure. In one embodiment a tertiary sulfonium containing substance may not comprise a heterocyclic ring structure. In one embodiment a tertiary sulfonium containing substance may not comprise a five membered ring structure. In one embodiment a tertiary sulfonium containing substance may not comprise a six membered ring structure. In one embodiment a tertiary sulfonium containing substance may not comprise a heterocyclic six membered ring structure. In one embodiment a tertiary sulfonium containing substance may not comprise a heterocyclic five membered ring structure. In one embodiment a tertiary sulfonium containing substance may not comprise a nitrogen heterocycle.

In one embodiment a tertiary sulfonium containing substance may not comprise a tertiary sulfonium cation that is at least part of a heterocyclic ring structure. In one embodiment a tertiary sulfonium containing substance may not comprise a tertiary sulfonium cation that is at least part of a five membered heterocyclic ring structure. In one embodiment a tertiary sulfonium containing substance may not comprise a tertiary sulfonium cation that is at least part of a six membered heterocyclic ring structure.

A non-limiting example of a tertiary sulfonium containing substance comprised of one or more tertiary sulfonium that may be used is dimethylsulfoniopropionate (DMSP), (also known as DMSP or dimethylpropiothetin), wherein DMSP may be substituted for or used in combination with one or more tertiary sulfonium containing substances described herein. One embodiment of the present invention may be a combination of substances comprising DMSP and another embodiment may also comprise at least one RNA substance. Other embodiments may be combinations of substances comprising DMSP wherein the concentration of DMSP may be one or more concentration in the stabilizing substance weight percent concentration list or in the stabilizing substance molar concentration range list or in the stabilizing substance molar concentration list.

A non-limiting example of a tertiary sulfonium containing substance comprised of one or more tertiary sulfonium that may be used is dimethylsulfonioacetate (DMSA) (also known as DMSA or (carboxymethyl)dimethylsulfonium), wherein DMSA may be substituted for or used in combination with one or more tertiary sulfonium containing substances described herein. One embodiment of the present invention may be a combination of substances comprising DMSA and another embodiment may also comprise at least one RNA substance. Other embodiments may be combinations of substances comprising DMSA wherein the concentration of DMSA may be one or more concentration in the stabilizing substance weight percent concentration list or in the stabilizing substance molar concentration range list or in the stabilizing substance molar concentration list.

A non-limiting example of a tertiary sulfonium containing substance comprised of one or more tertiary sulfonium that may be used is S-methylmethionine (SMM), wherein SMM may be substituted for or used in combination with one or more tertiary sulfonium containing substances described herein. One embodiment of the present invention may be a combination of substances comprising SMM and another embodiment may also comprise at least one RNA substance. Other embodiments may be combinations of substances comprising SMM wherein the concentration of SMM may be one or more concentration in the stabilizing substance weight percent concentration list or in the stabilizing substance molar concentration range list or in the stabilizing substance molar concentration list.

For conciseness, the following list of tertiary sulfonium containing substances, is herein referred to as the tertiary sulfonium containing substance list wherein one or more of the following substances may be substituted for or used in combination with one or more tertiary sulfonium containing substance as described herein.

In other embodiments, other tertiary sulfonium containing substances that may be substituted for or used in combination with one or more tertiary sulfonium containing substance as described herein may comprise one or more of the following, including but not limited to: DMSP, DMSA, SMM, 4-dimethylsulphonio-2-hydroxybutyrate, 4-dimethylsulfonio-2-hydroxybutyrate, ethylmethylsulfoniopropionate, diethylsulfoniopropionate, isopropylmethylsulfoniopropionate, tetramethylenesulfoniopropionate, methylpropylsulfoniopropionate, dimethylsulfonioacetate, 2-dimethylsulfonioethanol, dimethylthioethanol, dimethylsulfoniopropionate-amine, dimethylsulfoniopropionate-aldehyde, methyl-dimethylsulfoniopropionate, 4-hydroxyphenyldimethylsulfonium, (2-carboxyethyl)(dimethyl)sulfonium, (ethoxycarbonylmethyl)dimethylsulfonium, dibutylsulfoniopropionate, butylethylsulfoniopropionate, butylmethylsulfoniopropionate, butylpropylsulfoniopropionate, diproprylsulfoniopropionate, diisopropylsulfoniopropionate, isopropylproprylsulfoniopropionate, isopropylethylsulfoniopropionate, isopropylbutylsulfoniopropionate, diethylsulfonioacetate, dibutylsulfonioacetate, ethylmethylsulfonioacetate, butylmethylsulfonioacetate, butylethylsulfonioacetate, dipropylsulfonioacetate, propylethylsulfonioacetate, porpylmethylsulfonioacetate, propylbutylsulfonioacetate, ispropylmethylsulfonioacetate, isopropylethylsulfonioacetate, isopropylbutylsulfonioacetate, isopropylpropylsulfonioacetate, diisopropylsulfonioacetate, sulfocholines, carboxythetins, sulfothetins, and sulfonium sulfonates, or salts, or derivatives, or combinations thereof.

Embodiments of the present invention may include combinations comprising one or more of the substances from the tertiary sulfonium containing substance list substituted for or used in combination with one or more tertiary sulfonium containing substance or one or more RNA stabilizing substance, such as an aprotic substance as a non-limiting example, and other embodiments may also comprise at least one RNA substance.

Other embodiments may include combinations of substances comprising one or more tertiary sulfonium containing substance selected from the tertiary sulfonium containing substance list wherein the concentration of one or more substances may be one or more concentration in the stabilizing substance weight percent concentration list or in the stabilizing substance molar concentration range list or in the stabilizing substance molar concentration list.

In one embodiment, a tertiary sulfonium containing substance may have a molecular weight between about 50-100,000 daltons. In one embodiment, a tertiary sulfonium containing substance may have a molecular weight between about 50-50,000 daltons. In one embodiment, a tertiary sulfonium containing substance may have a molecular weight between about 50-20,000 daltons. In one embodiment, a tertiary sulfonium containing substance may have a molecular weight between about 50-10,000 daltons. In one embodiment, a tertiary sulfonium containing substance may have a molecular weight between about 50-5,000 daltons. In one embodiment, a tertiary sulfonium containing substance may have a molecular weight between about 50-2,000 daltons. In one embodiment, a tertiary sulfonium containing substance may have a molecular weight between about 50-1,000 daltons. In one embodiment, a tertiary sulfonium containing substance may have a molecular weight between about 50-900 daltons. In one embodiment, a tertiary sulfonium containing substance may have a molecular weight between about 50-800 daltons. In one embodiment, a tertiary sulfonium containing substance may have a molecular weight between about 50-700 daltons. In one embodiment, a tertiary sulfonium containing substance may have a molecular weight between about 50-600 daltons. In one embodiment, a tertiary sulfonium containing substance may have a molecular weight between about 50-500 daltons. In one embodiment, a tertiary sulfonium containing substance may have a molecular weight between about 50-400 daltons. In one embodiment, a tertiary sulfonium containing substance may have a molecular weight between about 50-300 daltons. In one embodiment, a tertiary sulfonium containing substance may have a molecular weight between about 50-250 daltons.

In one embodiment, a tertiary sulfonium containing substance may have a molecular weight between about 100-100,000 daltons. In one embodiment, a tertiary sulfonium containing substance may have a molecular weight between about 100-50,000 daltons. In one embodiment, a tertiary sulfonium containing substance may have a molecular weight between about 100-20,000 daltons. In one embodiment, a tertiary sulfonium containing substance may have a molecular weight between about 100-10,000 daltons In one embodiment, a tertiary sulfonium containing substance may have a molecular weight between about 100-5,000 daltons. In one embodiment, a tertiary sulfonium containing substance may have a molecular weight between about 100-2,000 daltons. In one embodiment, a tertiary sulfonium containing substance may have a molecular weight between about 100-1,000 daltons. In one embodiment, a tertiary sulfonium containing substance may have a molecular weight between about 100-900 daltons. In one embodiment, a tertiary sulfonium containing substance may have a molecular weight between about 100-800 daltons. In one embodiment, a tertiary sulfonium containing substance may have a molecular weight between about 100-700 daltons. In one embodiment, a tertiary sulfonium containing substance may have a molecular weight between about 100-600 daltons. In one embodiment, a tertiary sulfonium containing substance may have a molecular weight between about 100-500 daltons. In one embodiment, a tertiary sulfonium containing substance may have a molecular weight between about 100-400 daltons. In one embodiment, a tertiary sulfonium containing substance may have a molecular weight between about 100-300 daltons. In one embodiment, a tertiary sulfonium containing substance may have a molecular weight between about 100-250 daltons.

One embodiment of the present invention is the method whereby one or more tertiary sulfonium containing substance may be combined, such as by mixing, with at least one or more RNA substance to produce a mixture comprising at least one or more tertiary sulfonium containing substance and at least one or more RNA substance. As a non-limiting example one or more RNA substance may be mixed with one or more tertiary sulfonium containing substance.

Another embodiment of the present invention is the method whereby one or more tertiary sulfonium containing substance may be combined, such as by mixing, with at least one or more RNA substance to produce a mixture comprising at least one or more RNA substance and at least one or more tertiary sulfonium containing substance at one or more of the RNA stabilizing substance concentrations within the stabilizing substance molar concentration range list or in the stabilizing substance molar concentration list or the stabilizing substance weight percent concentration list as described herein. These same methods may be used to combine one or more other substances, including, but not limited to one or more cellular uptake agents or one or more additional RNA stabilizing substances, with one or more RNA substances and one or more tertiary sulfonium containing substances to produce a mixture comprising one or more RNA substance, one or more tertiary sulfonium containing substance, and one or more cellular uptake agent or one or more additional RNA stabilizing substance.

One embodiment of the present invention is the method whereby one or more tertiary sulfonium containing substance may be combined, such as by mixing, with at least one or more RNA substance to produce a composition comprising at least one or more RNA substance and at least one or more tertiary sulfonium containing substance. Another embodiment of the present invention is the method whereby one or more tertiary sulfonium containing substance may be combined, such as by mixing, with one or more RNA substance to produce a composition comprising at least one or more RNA substance and at least one or more tertiary sulfonium containing substance at one or more of the RNA stabilizing substance concentrations within the stabilizing substance molar concentration range list or in the stabilizing substance molar concentration list or stabilizing substance weight percent concentration list as described herein. These same methods may be used to combine one or more other substances, including, but not limited to one or more cellular uptake agents or one or more additional RNA stabilizing substance, with one or more RNA substance and one or more tertiary sulfonium containing substance to produce a composition comprising one or more RNA substance, one or more tertiary sulfonium containing substance, and one or more cellular uptake agent or one or more additional RNA stabilizing substance.

In one embodiment a tertiary sulfonium containing substance may be monomeric. In one embodiment a tertiary sulfonium containing substance may be aliphatic. In one embodiment a tertiary sulfonium containing substance may comprise a ring structure. In another embodiment a tertiary sulfonium containing substance may comprise a heterocyclic ring structure. In one embodiment a tertiary sulfonium containing substance may comprise an aromatic ring structure. In another embodiment a tertiary sulfonium containing substance may comprise an aromatic heterocyclic ring structure.

In one embodiment a tertiary sulfonium containing substance may not comprise a ring structure. In one embodiment a tertiary sulfonium containing substance may not comprise a heterocyclic ring structure. In one embodiment a tertiary sulfonium containing substance may not comprise an aromatic ring structure. In one embodiment a tertiary sulfonium containing substance may not comprise an aromatic heterocyclic ring structure.

In one embodiment a tertiary sulfonium containing substance may comprise a cationic moiety at about physiologic pH. In one embodiment a tertiary sulfonium containing substance may comprise an anionic moiety at about physiologic pH. In one embodiment a tertiary sulfonium containing substance may comprise a zwitterion at about physiologic pH.

In one embodiment a tertiary sulfonium containing substance may comprise one or more PIF as described herein.

In one embodiment a tertiary sulfonium containing substance may be monomeric. In one embodiment a tertiary sulfonium containing substance may be polymeric. In embodiment a tertiary sulfonium containing substance may comprise a polymer.

In one embodiment, one or more tertiary sulfonium containing substance may be at least part of a polymer comprised of at least one or more tertiary sulfonium containing substances.

In one embodiment one or more composition comprising one or more RNA substance and one or more tertiary sulfonium containing substance may comprise one or more water concentrations in the water concentration list or in the water concentration range list. In one embodiment one or more composition comprising one or more RNA substance, one or more tertiary sulfonium containing substance, and one or more additional substance, such as one or more cellular uptake agent or one or more additional RNA stabilizing substance as non-limiting examples, may comprise one or more water concentrations in the water concentration list or in the water concentration range list. In one embodiment one or more composition comprising one or more RNA substance and one or more tertiary sulfonium containing substance may have a dielectric constant of one or more dielectric constant in the dielectric constant list or in the dielectric constant range list. In one embodiment one or more composition comprising one or more RNA substance, one or more tertiary sulfonium containing substance, and one or more additional substance, such as one or more cellular uptake agent or one or more additional RNA stabilizing substance as non-limiting examples, may have a dielectric constant of one or more dielectric constant in the dielectric constant list or in the dielectric constant range list.

Embodiments of the present invention may include compositions comprising one or more tertiary sulfonium containing substance used in a composition comprising one or more RNA substance, one or more tertiary sulfonium containing substance, and one or more of the following substances: water, cellular uptake agents, aprotic substances, low dielectric substances, cyclic phosphate or metaphosphate containing substances, non-carbohydrate organic osmolyte substances, quaternary ammonium containing substances, quaternary phosphonium containing substances, hydrotrope containing substances, surfactant containing substances, betaine containing substances, stabilizing monomers, stabilizing polymers, or supplemental RNA stabilizing substances. Other embodiments may include one or more composition comprising one or more tertiary sulfonium containing substance and one or more RNA substance and may also comprise one or more cellular uptake agent or one or more additional RNA stabilizing substances. Other embodiments may include compositions comprising one or more tertiary sulfonium containing substance, one or more RNA substance, and one or more additional substances, such as: one or more cellular uptake agent, or one or more additional RNA stabilizing substance, or one or more buffering agent, or one or more chelating agent, or one or more inorganic salt, or water, as non-limiting examples.

Embodiments of the present invention may include one or more compositions comprising one or more RNA substance and one or more tertiary sulfonium containing substance, wherein a composition is not lyophilized.

In one embodiment one or more composition comprising one or more RNA substance and one or more tertiary sulfonium containing substance may not be lyophilized. In one embodiment one or more composition comprising one or more RNA substance, one or more tertiary sulfonium containing substance, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more tertiary sulfonium containing substance, wherein a composition may be lyophilized.

In one embodiment one or more composition comprising one or more RNA substance and one or more tertiary sulfonium containing substance may be lyophilized. In one embodiment one or more composition comprising one or more RNA substance, one or more tertiary sulfonium containing substance, and one or more additional substance, such as one or more cellular uptake agent or one or more additional RNA stabilizing substance as non-limiting examples, may be lyophilized.

Embodiments of the present invention may include one or more compositions comprising one or more tertiary sulfonium containing substance and one or more RNA substance described herein, wherein one or more composition described herein may have a melting of one or more melting point in the stabilizing composition melting point list.

In one embodiment one or more composition comprising one or more RNA substance and one or more tertiary sulfonium containing substance may have a melting point of one or more melting point in the stabilizing composition melting point list. In one embodiment one or more composition comprising one or more RNA substance, one or more tertiary sulfonium containing substance, and one or more additional substance, such as one or more cellular uptake agent or one or more additional RNA stabilizing substance as a non-limiting example, may have a melting point of one or more melting point in the stabilizing composition melting point list.

### Quaternary Ammonium Substances:

The inventors have discovered that quaternary ammonium containing substances may stabilize RNA substances.

The inventors have discovered that RNA stabilizing substances may comprise quaternary ammonium containing substances. The inventors have also discovered that the stability of RNA substances may be enhanced in an RNA storage environment comprising a quaternary ammonium containing substance. The inventors have also discovered that the stability of RNA substances may be enhanced in an RNA storage environment comprising a quaternary ammonium containing substance and one or more additional RNA stabilizing substance, such as an aprotic substance.

The inventors have also discovered that the stability of RNA substances may be enhanced in compositions comprising an RNA substance and a quaternary ammonium containing substance. The inventors have also discovered that the stability of RNA substances may be enhanced in compositions comprising an RNA substance, a quaternary ammonium containing substance, and one or more additional RNA stabilizing substance, such as an aprotic substance.

In one embodiment of the present invention, one or more RNA stabilizing substance may comprise a quaternary ammonium containing substance.

One embodiment of the present invention may include a composition comprised of one or more RNA substance and one or more quaternary ammonium containing substance. Another embodiment of the present invention may include a composition comprised of one or more RNA substance, one or more quaternary ammonium containing substance, and one or more additional RNA stabilizing substance, such as an aprotic substance.

One embodiment of the present invention may include a combination or mixture comprising one or more RNA substance and one or more quaternary ammonium containing substance. Another embodiment of the present invention may include a combination or mixture comprising one or more RNA substance, one or more quaternary ammonium containing substance, and one or more additional RNA stabilizing substance, such as an aprotic substance.

Embodiments of the present invention that comprise one or more RNA substance and one or more quaternary ammonium containing substance may include combining, such as by mixing, one or more RNA substance with one or more substance that comprises at least one or more quaternary ammonium containing substance.

Embodiments of the present invention that comprise one or more RNA substance, one or more quaternary ammonium containing substance, and one or more additional RNA stabilizing substance, such as an aprotic substance, may include combining, such as by mixing, one or more RNA substance with one or more substance that comprises at least one or more quaternary ammonium containing substance and one or more substance that comprises at least one or more RNA stabilizing substance, such as an aprotic substance.

An embodiment of the present invention may include compositions of materials that comprise one or more RNA substance and at least one or more quaternary ammonium containing substance. The storage environment that improves the stability of RNA substances may comprise at least one or more vapor, liquid, powder, or solid quaternary ammonium containing substance.

An embodiment of the present invention may include compositions of materials that comprise one or more RNA substance, one or more quaternary ammonium containing substance, and one or more additional RNA stabilizing substance, such as an aprotic substance. The storage environment that improves the stability of RNA substances may comprise at least one or more vapor, liquid, powder, or solid quaternary ammonium containing substance and may comprise at least one or more vapor, liquid, powder, or solid RNA stabilizing substance, such as an aprotic substance.

An embodiment of the present invention may include combinations of materials that comprise one or more RNA substance and at least one or more quaternary ammonium containing substance. The storage environment that improves the stability of RNA substances may comprise at least one or more vapor, liquid, powder, or solid quaternary ammonium containing substance.

An embodiment of the present invention may include combinations of materials that comprise one or more RNA substance, one or more quaternary ammonium containing substance, and one or more additional RNA stabilizing substance, such as an aprotic substance. The storage environment that improves the stability of RNA substances may comprise at least one or more vapor, liquid, powder, or solid quaternary ammonium containing substance and may comprise at least one or more vapor, liquid, powder, or solid RNA stabilizing substance, such as an aprotic substance.

In one embodiment of the present invention a composition comprised of one or more RNA substance and one or more quaternary ammonium containing substance, produces a mixture with at least one or more RNA substance and at least one or more quaternary ammonium containing substance. In one embodiment of the present invention a composition comprised of one or more RNA substance, one or more quaternary ammonium containing substance, and one or more additional RNA stabilizing substance, such as an aprotic substance, produces a mixture with at least one or more RNA substance, at least one or more quaternary ammonium containing substance, and at least one or more additional RNA stabilizing substance, such as an aprotic substance.

In one embodiment of the present invention a combination comprising one or more RNA substance and one or more quaternary ammonium containing substance, produces a mixture with at least one or more RNA substance and at least one or more quaternary ammonium containing substance. In one embodiment of the present invention a combination comprising one or more RNA substance, one or more quaternary ammonium containing substance, and one or more additional RNA stabilizing substance, such as an aprotic substance, produces a mixture with at least one or more RNA substance, at least one or more quaternary ammonium containing substance, and at least one or more additional RNA stabilizing substance, such as an aprotic substance.

In one embodiment of the present invention a composition with improved RNA stability may comprise one or more RNA substance and one or more quaternary ammonium containing substance. In one embodiment of the present invention a composition with improved RNA stability may comprise one or more RNA substance, one or more quaternary ammonium containing substance, and one or more additional RNA stabilizing substance, such as an aprotic substance.

In one embodiment of the present invention a composition with improved RNA stability may comprise a mixture of one or more RNA substance and one or more quaternary ammonium containing substance. In one embodiment of the present invention a composition with improved RNA stability may comprise a mixture of one or more RNA substance, one or more quaternary ammonium containing substance, and one or more additional RNA stabilizing substance, such as an aprotic substance.

In one embodiment of the present invention, each component included in a composition comprising one or more RNA substance and one or more quaternary ammonium containing substance, may be stored separately, such as in a kit, or such as individual substances or as mixtures of one or more substance, and then combined later to produce a composition comprising one or more RNA substance and one or more quaternary ammonium containing substance.

In one embodiment of the present invention, each component included in a composition comprising one or more RNA substance, one or more quaternary ammonium containing substance, and one or more additional RNA stabilizing substance, such as an aprotic substance, may be stored separately, such as in a kit, or such as individually or as mixtures of one or more substance, and then combined later to produce a composition comprising one or more RNA substance, one or more quaternary ammonium containing substance, and one or more additional RNA stabilizing substance, such as an aprotic substance.

In one embodiment of the present invention a composition comprising one or more RNA substance and one or more quaternary ammonium containing substance may be at least partially biocompatible.

In one embodiment of the present invention a composition comprising one or more RNA substance, one or more quaternary ammonium containing substance, and one or more additional RNA stabilizing substance, such as an aprotic substance, may be at least partially biocompatible.

In one embodiment of the present invention a combination comprised of one or more RNA substance and one or more quaternary ammonium containing substance may be at least partially biocompatible.

In one embodiment of the present invention a combination comprised of one or more RNA substance, one or more quaternary ammonium containing substance, and one or more additional RNA stabilizing substance, such as an aprotic substance, may be at least partially biocompatible.

Embodiments of the present invention comprising at least one or more quaternary ammonium containing substances may include one or more forms of the substance. These forms may include, but are not limited to, one more counterions, ionic forms, conjugate bases, conjugate acids, protonated or deprotonated forms, hydrated or dehydrated forms, isomers, structural isomers, stereo isomers, chiral forms, salts, or combinations thereof.

In one embodiment a quaternary ammonium containing substance may comprise one or more quaternary ammonium cation. In one embodiment a quaternary ammonium containing substance may comprise 1 or more quaternary ammonium cations, 2 or more quaternary ammonium cations, 3 or more quaternary ammonium cations, 4 or more quaternary ammonium cations, 5 or more quaternary ammonium cations, 10 or more quaternary ammonium cations, 50 or more quaternary ammonium cations, or 100 or more quaternary ammonium cations. In one embodiment a quaternary ammonium containing substance may comprise between about 1-1,000 quaternary ammonium cations, or between about 1-500 quaternary ammonium cations, or between about 1-200 quaternary ammonium cations, or between about 1-100 quaternary ammonium cations, or between about 1-50 quaternary ammonium cations, or between about 1-20 quaternary ammonium cations, or between about 1-10 quaternary ammonium cations, or between about 1-5 quaternary ammonium cations, or between about 1-3 quaternary ammonium cations, or between about 1-2 quaternary ammonium cations, or between about 2-1,000 quaternary ammonium cations, or between about 2-500 quaternary ammonium cations, or between about 2-200 quaternary ammonium cations, or between about 2-100 quaternary ammonium cations, or between about 2-50 quaternary ammonium cations, or between about 2-20 quaternary ammonium cations, or between about 2-10 quaternary ammonium cations, or between about 2-5 quaternary ammonium cations, or between about 2-3 quaternary ammonium cations.

In one embodiment a quaternary ammonium containing substance may comprise one or more aliphatic quaternary ammonium. In one embodiment a quaternary ammonium containing substance may comprise one or more quaternary ammonium nitrogen heterocycle. In one embodiment a quaternary ammonium containing substance may comprise one or more quaternary ammonium aromatic nitrogen heterocycle. In one embodiment a quaternary ammonium containing substance may comprise one or more five membered quaternary ammonium nitrogen heterocycle. In one embodiment a quaternary ammonium containing substance may comprise one or more six membered quaternary ammonium nitrogen heterocycle. In one embodiment a quaternary ammonium containing substance may comprise one or more five membered aromatic quaternary ammonium nitrogen heterocycle. In one embodiment a quaternary ammonium containing substance may comprise one or more six membered aromatic quaternary ammonium nitrogen heterocycle.

In one embodiment a quaternary ammonium containing substance may comprise one or more cation or cationic moiety. In one embodiment a quaternary ammonium containing substance may comprise one or more anion or anionic moiety. In one embodiment a quaternary ammonium containing substance may comprise one or more zwitterion or zwitterionic moiety. In one embodiment a quaternary ammonium containing substance may be zwitterionic.

In one embodiment a quaternary ammonium containing substance may comprise one or more betaine, such as a carboxybetaine as a non-limiting example. In one embodiment a quaternary ammonium containing substance may comprise one or more sulfobetaine, such as a non-detergent sulfobetaine as a non-limiting example.

In one embodiment a quaternary ammonium containing substance may comprise one or more quaternary ammonium cation that may be at least part of one or more nitrogen heterocycle or aromatic nitrogen heterocycle, such as a pyridinium, pyrrolidinium, imidazolium, piperidinium, indolium, pyrimidinium, or purinium group as non-limiting examples.

In one embodiment a quaternary ammonium containing substance may comprise one or more carboxylate group. In one embodiment a quaternary ammonium containing substance may comprise one or more carboxylate ester. In one embodiment a quaternary ammonium containing substance may comprise one or more sulfonate group. In one embodiment a quaternary ammonium containing substance may comprise one or more organophosphate group. In one embodiment a quaternary ammonium containing substance may comprise one or more organosulfate group.

In one embodiment a quaternary ammonium containing substance may comprise one or more carboxylate anion or anionic moiety. In one embodiment a quaternary ammonium containing substance may comprise one or more sulfonate anion or anionic moiety. In one embodiment a quaternary ammonium containing substance may comprise one or more organophosphate anion or anionic moiety. In one embodiment a quaternary ammonium containing substance may comprise one or more organosulfate anion or anionic moiety.

In one embodiment a quaternary ammonium containing substance may be hydroxylated. In one embodiment a quaternary ammonium containing substance may comprise one or more hydroxy groups. In one embodiment a quaternary ammonium containing substance may comprise 1 or more hydroxy groups, 2 or more hydroxy groups, 3 or more hydroxy groups, 4 or more hydroxy groups, 5 or more hydroxy groups, 10 or more hydroxy groups, or 50 or more hydroxy groups. In one embodiment a quaternary ammonium containing substance may comprise between about 1-100 hydroxy groups, or between about 1-50 hydroxy groups, or between about 1-20 hydroxy groups, or between about 1-10 hydroxy groups, or between about 1-5 hydroxy groups, or between about 1-3 hydroxy groups, or between about 2-100 hydroxy groups, or between about 2-50 hydroxy groups, or between about 2-20 hydroxy groups, or between about 2-10 hydroxy groups, or between about 2-5 hydroxy groups, or between about 2-3 hydroxy groups.

In one embodiment a quaternary ammonium containing substance may be carboxylated. In one embodiment a quaternary ammonium containing substance may comprise one or more carboxylate or carboxylic acid groups. In one embodiment a quaternary ammonium containing substance may comprise 1 or more carboxylate or carboxylic acid groups, 2 or more carboxylate or carboxylic acid groups, 3 or more carboxylate or carboxylic acid groups, 4 or more carboxylate or carboxylic acid groups, 5 or more carboxylate or carboxylic acid groups, 10 or more carboxylate or carboxylic acid groups, or 50 or more carboxylate or carboxylic acid groups. In one embodiment a quaternary ammonium containing substance may comprise between about 1-100 carboxylate or carboxylic acid groups, or between about 1-50 carboxylate or carboxylic acid groups, or between about 1-20 carboxylate or carboxylic acid groups, or between about 1-10 carboxylate or carboxylic acid groups, or between about 1-5 carboxylate or carboxylic acid groups, or between about 1-3 carboxylate or carboxylic acid groups, or between about 2-100 carboxylate or carboxylic acid groups, or between about 2-50 carboxylate or carboxylic acid groups, or between about 2-20 carboxylate or carboxylic acid groups, or between about 2-10 carboxylate or carboxylic acid groups, or between about 2-5 carboxylate or carboxylic acid groups, or between about 2-3 carboxylate or carboxylic acid groups.

In one embodiment a quaternary ammonium containing substance may comprise one or more ester, such as one or more carboxylate ester or carbonate ester as non-limiting examples. In one embodiment a quaternary ammonium containing substance may comprise one or more choline-based ester. In one embodiment a quaternary ammonium containing substance may comprise 1 or more esters, 2 or more esters, 3 or more esters, 4 or more esters, 5 or more esters, or more esters, or 50 or more esters. In one embodiment a quaternary ammonium containing substance may comprise between about 1-100 esters, or between about 1-50 esters, or between about 1-20 esters, or between about 1-10 esters, or between about 1-5 esters, or between about 1-3 esters, or between about 2-100 esters, or between about 2-50 esters, or between about 2-20 esters, or between about 2-10 esters, or between about 2-5 esters, or between about 2-3 esters

In one embodiment a quaternary ammonium containing substance may comprise one or more ester bond. In one embodiment a quaternary ammonium containing substance may comprise one or more choline-based ester. In one embodiment a quaternary ammonium containing substance may comprise 1 or more ester bonds, 2 or more ester bonds, 3 or more ester bonds, 4 or more ester bonds, 5 or more ester bonds, 10 or more ester bonds, or 50 or more ester bonds. In one embodiment a quaternary ammonium containing substance may comprise between about 1-100 ester bonds, or between about 1-50 ester bonds, or between about 1-20 ester bonds, or between about 1-10 ester bonds, or between about 1-5 ester bonds, or between about 1-3 ester bonds, or between about 2-100 ester bonds, or between about 2-50 ester bonds, or between about 2-20 ester bonds, or between about 2-10 ester bonds, or between about 2-5 ester bonds, between about 2-3 ester bonds.

In one embodiment a quaternary ammonium containing substance may comprise one or more ether bond. In one embodiment a quaternary ammonium containing substance may comprise 1 or more ether bonds, 2 or more ether bonds, 3 or more ether bonds, 4 or more ether bonds, 5 or more ether bonds, 10 or more ether bonds, or 50 or more ether bonds. In one embodiment a quaternary ammonium containing substance may comprise between about 1-100 ether bonds, or between about 1-50 ether bonds, or between about 1-20 ether bonds, or between about 1-10 ether bonds, or between about 1-5 ether bonds, or between about 1-3 ether bonds, or between about 2-100 ether bonds, or between about 2-50 ether bonds, or between about 2-20 ether bonds, or between about 2-10 ether bonds, or between about 2-5 ether bonds, or between about 2-3 ether bonds.

In one embodiment a quaternary ammonium containing substance may comprise one or more ether. In one embodiment a quaternary ammonium containing substance may comprise 1 or more ethers, 2 or more ethers, 3 or more ethers, 4 or more ethers, 5 or more ethers, 10 or more ethers, or 50 or more ethers. In one embodiment a quaternary ammonium containing substance may comprise between about 1-100 ethers, or between about 1-50 ethers, or between about 1-20 ethers, or between about 1-10 ethers, or between about 1-5 ethers, or between about 1-3 ethers, or between about 2-100 ethers, or between about 2-50 ethers, or between about 2-20 ethers, or between about 2-10 ethers, or between about 2-5 ethers, or between about 2-3 ethers.

In one embodiment a quaternary ammonium containing substance may comprise one or more amide bond. In one embodiment a quaternary ammonium containing substance may comprise 1 or more amide bonds, 2 or more amide bonds, 3 or more amide bonds, 4 or more amide bonds, 5 or more amide bonds, 10 or more amide bonds, or 50 or more amide bonds. In one embodiment a quaternary ammonium containing substance may comprise between about 1-100 amide bonds, or between about 1-50 amide bonds, or between about 1-20 amide bonds, or between about 1-10 amide bonds, or between about 1-5 amide bonds, or between about 1-3 amide bonds, or between about 2-100 amide bonds, or between about 2-50 amide bonds, or between about 2-20 amide bonds, or between about 2-10 amide bonds, or between about 2-5 amide bonds, or between about 2-3 amide bonds.

In one embodiment a quaternary ammonium containing substance may comprise one or more amide group. In one embodiment a quaternary ammonium containing substance may comprise 1 or more amide groups, 2 or more amide groups, 3 or more amide groups, 4 or more amide groups, 5 or more amide groups, 10 or more amide groups, or 50 or more amide groups. In one embodiment a quaternary ammonium containing substance may comprise between about 1-100 amide groups, or between about 1-50 amide groups, or between about 1-20 amide groups, or between about 1-10 amide groups, or between about 1-5 amide groups, or between about 1-3 amide groups, or between about 2-100 amide groups, or between about 2-50 amide groups, or between about 2-20 amide groups, or between about 2-10 amide groups, or between about 2-5 amide groups, or between about 2-3 amide groups.

In one embodiment a quaternary ammonium containing substance may comprise one or more organosulfate group. In one embodiment a quaternary ammonium containing substance may comprise 1 or more organosulfate groups, 2 or more organosulfate groups, 3 or more organosulfate groups, 4 or more organo sulfate groups, 5 or more organosulfate groups, 10 or more organosulfate groups, or 50 or more organosulfate groups. In one embodiment a quaternary ammonium containing substance may comprise between about 1-100 organosulfate groups, or between about 1-50 organosulfate groups, or between about 1-20 organosulfate groups, or between about 1-10 organosulfate groups, or between about 1-5 organosulfate groups, or between about 1-3 organosulfate groups, or between about 2-100 organosulfate groups, or between about 2-50 organosulfate groups, or between about 2-20 organosulfate groups, or between about 2-10 organosulfate groups, or between about 2-5 organosulfate groups, or between about 2-3 organosulfate groups.

In one embodiment a quaternary ammonium containing substance may comprise one or more sulfonate group. In one embodiment a quaternary ammonium containing substance may comprise 1 or more sulfonate groups, 2 or more sulfonate groups, 3 or more sulfonate groups, 4 or more sulfonate groups, 5 or more sulfonate groups, 10 or more sulfonate groups, or 50 or more sulfonate groups. In one embodiment a quaternary ammonium containing substance may comprise between about 1-100 sulfonate groups, or between about 1-50 sulfonate groups, or between about 1-20 sulfonate groups, or between about 1-10 sulfonate groups, or between about 1-5 sulfonate groups, or between about 1-3 sulfonate groups, or between about 2-100 sulfonate groups, or between about 2-50 sulfonate groups, or between about 2-20 sulfonate groups, or between about 2-10 sulfonate groups, or between about 2-5 sulfonate groups, or between about 2-3 sulfonate groups.

In one embodiment a quaternary ammonium containing substance may comprise one or more organophosphate group. In one embodiment a quaternary ammonium containing substance may comprise 1 or more organophosphate groups, 2 or more organophosphate groups, 3 or more organophosphate groups, 4 or more organophosphate groups, 5 or more organophosphate groups, or more organophosphate groups, or 50 or more organophosphate groups. In one embodiment a quaternary ammonium containing substance may comprise between about 1-100 organophosphate groups, or between about 1-50 organophosphate groups, or between about 1-20 organophosphate groups, or between about 1-10 organophosphate groups, or between about 1-5 organophosphate groups, or between about 1-3 organophosphate groups, or between about 2-100 organophosphate groups, or between about 2-50 organophosphate groups, or between about 2-20 organophosphate groups, or between about 2-10 organophosphate groups, or between about 2-5 organophosphate groups, or between about 2-3 organophosphate groups.

In one embodiment a quaternary ammonium containing substance may not comprise a hydroxy group. In one embodiment a quaternary ammonium containing substance may not comprise a carboxylate or carboxylic acid group. In one embodiment a quaternary ammonium containing substance may not comprise an ester bond. In one embodiment a quaternary ammonium containing substance may not comprise an ester, such as a carboxylate ester or carbonate ester as non-limiting examples. In one embodiment a quaternary ammonium containing substance may not comprise an ether. In one embodiment a quaternary ammonium containing substance may not comprise an ether bond. In one embodiment a quaternary ammonium containing substance may not comprise an organosulfate group. In one embodiment a quaternary ammonium containing substance may not comprise a sulfonate group. In one embodiment a quaternary ammonium containing substance may not comprise an organophosphate group.

In one embodiment a quaternary ammonium containing substance may comprise one or more polymer. In one embodiment a quaternary ammonium containing substance may be polymeric. In one embodiment a quaternary ammonium containing substance may comprise one or more cation. In one embodiment a quaternary ammonium containing substance may be cationic. In one embodiment a quaternary ammonium containing substance may be polycationic. In one embodiment a quaternary ammonium containing substance may comprise one or more anion. In one embodiment a quaternary ammonium containing substance may comprise one or more zwitterion. In one embodiment a quaternary ammonium containing substance may be zwitterionic.

In one embodiment a quaternary ammonium containing substance may comprise one or more ring structure. In one embodiment a quaternary ammonium containing substance may comprise one or more heterocyclic ring structure. In one embodiment a quaternary ammonium containing substance may comprise one or more five membered ring structure. In one embodiment a quaternary ammonium containing substance may comprise one or more six membered ring structure. In one embodiment a quaternary ammonium containing substance may comprise one or more heterocyclic six membered ring structure. In one embodiment a quaternary ammonium containing substance may comprise one or more heterocyclic five membered ring structure. In one embodiment a quaternary ammonium containing substance may comprise one or more nitrogen heterocycles.

In one embodiment a quaternary ammonium containing substance may comprise one or more aromatic ring structure. In one embodiment a quaternary ammonium containing substance may comprise one or more aromatic heterocyclic ring structure. In one embodiment a quaternary ammonium containing substance may comprise one or more aromatic five membered ring structure. In one embodiment a quaternary ammonium containing substance may comprise one or more aromatic six membered ring structure. In one embodiment a quaternary ammonium containing substance may comprise one or more aromatic heterocyclic six membered ring structure. In one embodiment a quaternary ammonium containing substance may comprise one or more aromatic heterocyclic five membered ring structure. In one embodiment a quaternary ammonium containing substance may comprise one or more aromatic nitrogen heterocycles.

In one embodiment a quaternary ammonium containing substance may comprise one or more quaternary ammonium cation that may be at least part of one or more nitrogen heterocycle, such as a five membered or six membered nitrogen heterocycle as non-limiting examples. In one embodiment a quaternary ammonium containing substance may comprise 1 or more quaternary ammonium cations that may be at least part of a five membered or six membered nitrogen heterocycle, 2 or more quaternary ammonium cations that may be at least part of a five membered or six membered nitrogen heterocycle, 3 or more quaternary ammonium cations that may be at least part of a five membered or six membered nitrogen heterocycle, 4 or more quaternary ammonium cations that may be at least part of a five membered or six membered nitrogen heterocycle, 5 or more quaternary ammonium cations that may be at least part of a five membered or six membered nitrogen heterocycle, 10 or more quaternary ammonium cations that may be at least part of a five membered or six membered nitrogen heterocycle, or 50 or more quaternary ammonium cations that may be at least part of a five membered or six membered nitrogen heterocycle. In one embodiment a quaternary ammonium containing substance may comprise between about 1-100 quaternary ammonium cations that may be at least part of a five membered or six membered nitrogen heterocycle, or between about 1-50 quaternary ammonium cations that may be at least part of a five membered or six membered nitrogen heterocycle, or between about 1-20 quaternary ammonium cations that may be at least part of a five membered or six membered nitrogen heterocycle, or between about 1-10 quaternary ammonium cations that may be at least part of a five membered or six membered nitrogen heterocycle, or between about 1-5 quaternary ammonium cations that may be at least part of a five membered or six membered nitrogen heterocycle, or between about 1-3 quaternary ammonium cations that may be at least part of a five membered or six membered nitrogen heterocycle, or between about 1-2 quaternary ammonium cations that may be at least part of a five membered or six membered nitrogen heterocycle, or between about 2-100 quaternary ammonium cations that may be at least part of a five membered or six membered nitrogen heterocycle, or between about 2-50 quaternary ammonium cations that may be at least part of a five membered or six membered nitrogen heterocycle, or between about 2-20 quaternary ammonium cations that may be at least part of a five membered or six membered nitrogen heterocycle, or between about 2-10 quaternary ammonium cations that may be at least part of a five membered or six membered nitrogen heterocycle, or between about 2-5 quaternary ammonium cations that may be at least part of a five membered or six membered nitrogen heterocycle, or between about 2-3 quaternary ammonium cations that may be at least part of a five membered or six membered nitrogen heterocycle.

In one embodiment a quaternary ammonium containing substance may comprise one or more pyridinium group. In one embodiment a quaternary ammonium containing substance may comprise one or more pyrrolidinium group. In one embodiment a quaternary ammonium containing substance may comprise one or more imidazolium group. In one embodiment a quaternary ammonium containing substance may comprise one or more piperidinium group. In one embodiment a quaternary ammonium containing substance may comprise one or more indolium group. In one embodiment a quaternary ammonium containing substance may comprise one or more pyrimidinium group. In one embodiment a quaternary ammonium containing substance may comprise one or more purinium group.

In one embodiment a quaternary ammonium containing substance may comprise one or more quaternary ammonium cation that may be at least part of one or more nitrogen heterocycle, such as a pyridinium, pyrrolidinium, imidazolium, piperidinium, indolium, pyrimidinium, or purinium group as non-limiting examples.

In one embodiment a quaternary ammonium containing substance may comprise 1 or more quaternary ammonium cations that may be at least part of a pyridinium, pyrrolidinium, imidazolium, piperidinium, indolium, pyrimidinium, or purinium group, 2 or more quaternary ammonium cations that may be at least part of a pyridinium, pyrrolidinium, imidazolium, piperidinium, indolium, pyrimidinium, or purinium group, 3 or more quaternary ammonium cations that may be at least part of a pyridinium, pyrrolidinium, imidazolium, piperidinium, indolium, pyrimidinium, or purinium group, 4 or more quaternary ammonium cations that may be at least part of a pyridinium, pyrrolidinium, imidazolium, piperidinium, indolium, pyrimidinium, or purinium group, 5 or more quaternary ammonium cations that may be at least part of a pyridinium, pyrrolidinium, imidazolium, piperidinium, indolium, pyrimidinium, or purinium group, 10 or more quaternary ammonium cations that may be at least part of a pyridinium, pyrrolidinium, imidazolium, piperidinium, indolium, pyrimidinium, or purinium group, or 50 or more quaternary ammonium cations that may be at least part of a pyridinium, pyrrolidinium, imidazolium, piperidinium, indolium, pyrimidinium, or purinium group. In one embodiment a quaternary ammonium containing substance may comprise between about 1-100 quaternary ammonium cations that may be at least part of a pyridinium, pyrrolidinium, imidazolium, piperidinium, indolium, pyrimidinium, or purinium group, or between about 1-50 quaternary ammonium cations that may be at least part of a pyridinium, pyrrolidinium, imidazolium, piperidinium, indolium, pyrimidinium, or purinium group, or between about 1-20 quaternary ammonium cations that may be at least part of a pyridinium, pyrrolidinium, imidazolium, piperidinium, indolium, pyrimidinium, or purinium group, or between about 1-10 quaternary ammonium cations that may be at least part of a pyridinium, pyrrolidinium, imidazolium, piperidinium, indolium, pyrimidinium, or purinium group, or between about 1-5 quaternary ammonium cations that may be at least part of a pyridinium, pyrrolidinium, imidazolium, piperidinium, indolium, pyrimidinium, or purinium group, or between about 1-3 quaternary ammonium cations that may be at least part of a pyridinium, pyrrolidinium, imidazolium, piperidinium, indolium, pyrimidinium, or purinium group, or between about 1-2 quaternary ammonium cations that may be at least part of a pyridinium, pyrrolidinium, imidazolium, piperidinium, indolium, pyrimidinium, or purinium group, or between about 2-100 quaternary ammonium cations that may be at least part of a pyridinium, pyrrolidinium, imidazolium, piperidinium, indolium, pyrimidinium, or purinium group, or between about 2-50 quaternary ammonium cations that may be at least part of a pyridinium, pyrrolidinium, imidazolium, piperidinium, indolium, pyrimidinium, or purinium group, or between about 2-20 quaternary ammonium cations that may be at least part of a pyridinium, pyrrolidinium, imidazolium, piperidinium, indolium, pyrimidinium, or purinium group, or between about 2-10 quaternary ammonium cations that may be at least part of a pyridinium, pyrrolidinium, imidazolium, piperidinium, indolium, pyrimidinium, or purinium group, or between about 2-5 quaternary ammonium cations that may be at least part of a pyridinium, pyrrolidinium, imidazolium, piperidinium, indolium, pyrimidinium, or purinium group, or between about 2-3 quaternary ammonium cations that may be at least part of a pyridinium, pyrrolidinium, imidazolium, piperidinium, indolium, pyrimidinium, or purinium group.

In one embodiment a quaternary ammonium containing substance may comprise one or more quaternary ammonium cation that may be at least part of a heterocyclic ring structure. In one embodiment a quaternary ammonium containing substance may comprise one or more quaternary ammonium cation that may be at least part of a five membered heterocyclic ring structure. In one embodiment a quaternary ammonium containing substance may comprise one or more quaternary ammonium cation that may be at least part of a six membered heterocyclic ring structure.

In one embodiment a quaternary ammonium containing substance may not be polymeric. In one embodiment a quaternary ammonium containing substance may not be polycationic. In one embodiment a quaternary ammonium containing substance may not comprise an anion. In one embodiment a quaternary ammonium containing substance may not comprise a zwitterion. In one embodiment a quaternary ammonium containing substance may not be zwitterionic.

In one embodiment a quaternary ammonium containing substance may not comprise a ring structure. In one embodiment a quaternary ammonium containing substance may not comprise a heterocyclic ring structure. In one embodiment a quaternary ammonium containing substance may not comprise a five membered ring structure. In one embodiment a quaternary ammonium containing substance may not comprise a six membered ring structure. In one embodiment a quaternary ammonium containing substance may not comprise a heterocyclic six membered ring structure. In one embodiment a quaternary ammonium containing substance may not comprise a heterocyclic five membered ring structure. In one embodiment a quaternary ammonium containing substance may not comprise a nitrogen heterocycle.

In one embodiment a quaternary ammonium containing substance may not comprise a quaternary ammonium cation that may be at least part of a heterocyclic ring structure. In one embodiment a quaternary ammonium containing substance may not comprise a quaternary ammonium cation that may be at least part of a five membered heterocyclic ring structure. In one embodiment a quaternary ammonium containing substance may not comprise a quaternary ammonium cation that may be at least part of a six membered heterocyclic ring structure. In one embodiment a quaternary ammonium containing substance may not comprise a quaternary ammonium cation that may be at least part of a nitrogen heterocycle.

In one embodiment a quaternary ammonium containing substance may not comprise an aromatic ring structure. In one embodiment a quaternary ammonium containing substance may not comprise an aromatic heterocyclic ring structure. In one embodiment a quaternary ammonium containing substance may not comprise an aromatic five membered ring structure. In one embodiment a quaternary ammonium containing substance may not comprise an aromatic six membered ring structure. In one embodiment a quaternary ammonium containing substance may not comprise an aromatic heterocyclic six membered ring structure. In one embodiment a quaternary ammonium containing substance may not comprise an aromatic heterocyclic five membered ring structure. In one embodiment a quaternary ammonium containing substance may not comprise an aromatic nitrogen heterocycle.

A non-limiting example of a quaternary ammonium containing substance comprised of one or more quaternary ammonium and one or more hydroxy group that may be used is choline, such as choline chloride as a non-limiting example, wherein choline may be substituted for or used in combination with one or more quaternary ammonium containing substances described herein. One embodiment of the present invention may be a combination of substances comprising choline and another embodiment may also comprise at least one RNA substance. Other embodiments may be combinations of substances comprising choline wherein the concentration of choline may be one or more concentration in the stabilizing substance weight percent concentration list or in the stabilizing substance molar concentration range list or in the stabilizing substance molar concentration list.

A non-limiting example of a quaternary ammonium containing substance comprised of one or more quaternary ammonium and one or more ester that may be used is acetylcholine, such as acetylcholine chloride as a non-limiting example, wherein acetylcholine may be substituted for or used in combination with one or more quaternary ammonium containing substances described herein. One embodiment of the present invention may be a combination of substances comprising acetylcholine and another embodiment may also comprise at least one RNA substance. Other embodiments may be combinations of substances comprising acetylcholine wherein the concentration of acetylcholine may be one or more concentration in the stabilizing substance weight percent concentration list or in the stabilizing substance molar concentration range list or in the stabilizing substance molar concentration list.

A non-limiting example of a quaternary ammonium containing substance comprised of one or more quaternary ammonium and one or more hydroxy group and one or more carboxylate or carboxylic acid group that may be used is carnitine, wherein carnitine may be substituted for or used in combination with one or more quaternary ammonium containing substances described herein. One embodiment of the present invention may be a combination of substances comprising carnitine and another embodiment may also comprise at least one RNA substance. Other embodiments may be combinations of substances comprising carnitine wherein the concentration of carnitine may be one or more concentration in the stabilizing substance weight percent concentration list or in the stabilizing substance molar concentration range list or in the stabilizing substance molar concentration list.

A non-limiting example of a quaternary ammonium containing substance comprised of one or more quaternary ammonium and one or more organophosphate group that may be used is phosphocholine, wherein phosphocholine may be substituted for or used in combination with one or more quaternary ammonium containing substances described herein. One embodiment of the present invention may be a combination of substances comprising phosphocholine and another embodiment may also comprise at least one RNA substance. Other embodiments may be combinations of substances comprising phosphocholine wherein the concentration of phosphocholine may be one or more concentration in the stabilizing substance weight percent concentration list or in the stabilizing substance molar concentration range list or in the stabilizing substance molar concentration list.

A non-limiting example of a quaternary ammonium containing substance comprised of one or more quaternary ammonium that may be used is tetraethylammonium, wherein tetraethylammonium may be substituted for or used in combination with one or more quaternary ammonium containing substances described herein. One embodiment of the present invention may be a combination of substances comprising tetraethylammonium and another embodiment may also comprise at least one RNA substance. Other embodiments may be combinations of substances comprising tetraethylammonium wherein the concentration of tetraethylammonium may be one or more concentration in the stabilizing substance weight percent concentration list or in the stabilizing substance molar concentration range list or in the stabilizing substance molar concentration list.

A non-limiting example of a quaternary ammonium containing substance comprised of one or more quaternary ammonium that may be used is decamethonium (also known as Syncurine), wherein decamethonium may be substituted for or used in combination with one or more quaternary ammonium containing substances described herein. One embodiment of the present invention may be a combination of substances comprising decamethonium and another embodiment may also comprise at least one RNA substance. Other embodiments may be combinations of substances comprising decamethonium wherein the concentration of decamethonium may be one or more concentration in the stabilizing substance weight percent concentration list or in the stabilizing substance molar concentration range list or in the stabilizing substance molar concentration list.

A non-limiting example of a quaternary ammonium containing substance comprised of one or more quaternary ammonium and one or more ester that may be used is poly(2-(trimethylamino)ethyl methacrylate) (PTMAEMA) (also known as, PTMAEMA, Poly(2-dimethylamino)ethyl methacrylate) methyl chloride quaternary salt, Poly(2-methacryloxyethyltrimethylammonium chloride), or MADQUAT), such as poly(2-dimethylamino)ethyl methacrylate) methyl chloride quaternary salt (˜8.5 kDa) as a non-limiting example, wherein PTMAEMA may be substituted for or used in combination with one or more quaternary ammonium containing substances described herein. One embodiment of the present invention may be a combination of substances comprising PTMAEMA and another embodiment may also comprise at least one RNA substance. Other embodiments may be combinations of substances comprising PTMAEMA wherein the concentration of PTMAEMA may be one or more concentration in the stabilizing substance weight percent concentration list or in the stabilizing substance molar concentration range list or in the stabilizing substance molar concentration list.

A non-limiting example of a quaternary ammonium containing substance comprised of one or more quaternary ammonium that may be used is hexadimethrine (also known as Polybrene), such as hexadimethrine bromide (˜5 kDa) as a non-limiting example, wherein hexadimethrine may be substituted for or used in combination with one or more quaternary ammonium containing substances described herein. One embodiment of the present invention may be a combination of substances comprising hexadimethrine and another embodiment may also comprise at least one RNA substance. Other embodiments may be combinations of substances comprising hexadimethrine wherein the concentration of hexadimethrine may be one or more concentration in the stabilizing substance weight percent concentration list or in the stabilizing substance molar concentration range list or in the stabilizing substance molar concentration list.

A non-limiting example of a quaternary ammonium containing substance comprised of one or more quaternary ammonium that may be used is poly(diallyldimethylammonium) (PDADMAC) (also known as, PDADMAC or poly(diallyldimethylammonium chloride)), such as poly(diallyldimethylammonium chloride) (˜8.5 kDa) as a non-limiting example, wherein PDADMAC may be substituted for or used in combination with one or more quaternary ammonium containing substances described herein. One embodiment of the present invention may be a combination of substances comprising PDADMAC and another embodiment may also comprise at least one RNA substance. Other embodiments may be combinations of substances comprising PDADMAC wherein the concentration of PDADMAC may be one or more concentration in the stabilizing substance weight percent concentration list or in the stabilizing substance molar concentration range list or in the stabilizing substance molar concentration list.

For conciseness, the following list of quaternary ammonium containing substances, is herein referred to as the quaternary ammonium containing substance list wherein one or more of the following substances may be substituted for or used in combination with one or more quaternary ammonium containing substance as described herein.

In other embodiments, other quaternary ammonium containing substances that may be substituted for or used in combination with one or more quaternary ammonium containing substance as described herein may comprise one or more of the following, including but not limited to: choline, phosphocholine, phosphorylcholine, acetylcholine, methacholine, butyrylcholine, ethylcholine, diethylcholine, succinylmonocholine, bethanechol, carbachol, carnitine, crotonobetaine, butyrobetaine, gamma-butyrobetaine, trimethylglycine, betaine, glycine betaine, alanine betaine, beta-alanine betaine, phosphobetaine, phosphorylbetaine, betaine phosphate, glycerophosphorylcholine, glycerophosphocholine, choline-O-sulfate, choline sulfate, hydroxyproline betaine, proline betaine, dimethyl proline, stachydrine, valine betaine, glutamic acid betaine, trigonelline, glutamine betaine, trimethyllysine, L-alpha glycerylphosphorylcholine (alpha-GPC), 4-trimethyl-ammoniobutanoate, trimethyl-ammoniobutanoate, pipecolic acid betaine, phosphobetaine, sulfobetaines, non-detergent sulfobetaines, dimethylethylammoniumpropane sulfonate, dimethylethylammonium-1-propane sulfonate, NDSB-195, 3-(1-pyridinio)-1-propane sulfonate, NDSB-201, NDSB-209, dimethyl-2-hydroxyethylammoniumpropane sulfonate, (2-hydroxyethyl)dimethyl(3-sulfopropyl)ammonium, 3-[dimethyl-(2-hydroxyethyl)ammonio]-1-propane sulfonate, NDSB-211, 3-(1-methylpiperidinium)-1-propane sulfonate, NDSB-221, NDSB-223, NDSB-225, NDSB-249, dimethylbenzylammonium propane sulfonate, 3-(benzyldimethylammonio)propanesulfonate, NDSB-256, 3-(4-tert-butyl-1-pyridinio)-1-propanesulfonate, NDSB-256-4T, 4-(3-butyl-1-imidazolio)-1-butanesulfonate, betaine aldehyde, imidazolium, piperidinium, pyridinium, pyrrolidinium, morpholinium, cholamine, tetramethonium, pentamethonium, tetraethylammonium, tetrapropylammonium, tetrabutylammonium, decamethonium, hexamethonium, hexadimethrine, polyquaterniums (including polyquaterniums 1-3, or polyquaterniums 5-9, or polyquaterniums 11-23, or polyquaterniums 25-28, or polyquaterniums 30-47, as non-limiting examples), poly(diallyldimethylammonium), poly(carboxybetaine methacrylate) ester, poly(carboxybetaine methacrylate), or poly(2-(trimethylamino)ethyl methacrylate), or salts, or derivatives, or combinations thereof.

Embodiments of the present invention may include combinations comprising one or more of the substances from the quaternary ammonium containing substance list substituted for or used in combination with one or more quaternary ammonium containing substance or one or more RNA stabilizing substance, such as an aprotic substance as a non-limiting example, and other embodiments may also comprise at least one RNA substance.

Other embodiments may include combinations of substances comprising one or more quaternary ammonium containing substance selected from the quaternary ammonium containing substance list wherein the concentration of one or more substances may be one or more concentration in the stabilizing substance weight percent concentration list or in the stabilizing substance molar concentration range list or in the stabilizing substance molar concentration list.

In one embodiment, a quaternary ammonium containing substance may have a molecular weight between about 50-1,000,000,000 daltons. In one embodiment, a quaternary ammonium containing substance may have a molecular weight between about 50-100,000,000 daltons. In one embodiment, a quaternary ammonium containing substance may have a molecular weight between about 50-10,000,000 daltons. In one embodiment, a quaternary ammonium containing substance may have a molecular weight between about 50-1,000,000 daltons. In one embodiment, a quaternary ammonium containing substance may have a molecular weight between about 50-500,000 daltons. In one embodiment, a quaternary ammonium containing substance may have a molecular weight between about 50-200,000 daltons. In one embodiment, a quaternary ammonium containing substance may have a molecular weight between about 50-100,000 daltons. In one embodiment, a quaternary ammonium containing substance may have a molecular weight between about 50-50,000 daltons. In one embodiment, a quaternary ammonium containing substance may have a molecular weight between about 50-20,000 daltons. In one embodiment, a quaternary ammonium containing substance may have a molecular weight between about 50-10,000 daltons. In one embodiment, a quaternary ammonium containing substance may have a molecular weight between about 50-5,000 daltons. In one embodiment, a quaternary ammonium containing substance may have a molecular weight between about 50-2,000 daltons. In one embodiment, a quaternary ammonium containing substance may have a molecular weight between about 50-1,000 daltons. In one embodiment, a quaternary ammonium containing substance may have a molecular weight between about 50-900 daltons. In one embodiment, a quaternary ammonium containing substance may have a molecular weight between about 50-800 daltons. In one embodiment, a quaternary ammonium containing substance may have a molecular weight between about 50-700 daltons. In one embodiment, a quaternary ammonium containing substance may have a molecular weight between about 50-600 daltons. In one embodiment, a quaternary ammonium containing substance may have a molecular weight between about 50-500 daltons. In one embodiment, a quaternary ammonium containing substance may have a molecular weight between about 50-400 daltons. In one embodiment, a quaternary ammonium containing substance may have a molecular weight between about 50-300 daltons. In one embodiment, a quaternary ammonium containing substance may have a molecular weight between about 50-250 daltons.

In one embodiment, a quaternary ammonium containing substance may have a molecular weight between about 100-1,000,000,000 daltons. In one embodiment, a quaternary ammonium containing substance may have a molecular weight between about 100-100,000,000 daltons. In one embodiment, a quaternary ammonium containing substance may have a molecular weight between about 100-10,000,000 daltons. In one embodiment, a quaternary ammonium containing substance may have a molecular weight between about 100-1,000,000 daltons. In one embodiment, a quaternary ammonium containing substance may have a molecular weight between about 100-500,000 daltons. In one embodiment, a quaternary ammonium containing substance may have a molecular weight between about 100-200,000 daltons. In one embodiment, a quaternary ammonium containing substance may have a molecular weight between about 100-100,000 daltons. In one embodiment, a quaternary ammonium containing substance may have a molecular weight between about 100-50,000 daltons. In one embodiment, a quaternary ammonium containing substance may have a molecular weight between about 100-20,000 daltons. In one embodiment, a quaternary ammonium containing substance may have a molecular weight between about 100-10,000 daltons. In one embodiment, a quaternary ammonium containing substance may have a molecular weight between about 100-5,000 daltons. In one embodiment, a quaternary ammonium containing substance may have a molecular weight between about 100-2,000 daltons. In one embodiment, a quaternary ammonium containing substance may have a molecular weight between about 100-1,000 daltons. In one embodiment, a quaternary ammonium containing substance may have a molecular weight between about 100-900 daltons. In one embodiment, a quaternary ammonium containing substance may have a molecular weight between about 100-800 daltons. In one embodiment, a quaternary ammonium containing substance may have a molecular weight between about 100-700 daltons. In one embodiment, a quaternary ammonium containing substance may have a molecular weight between about 100-600 daltons. In one embodiment, a quaternary ammonium containing substance may have a molecular weight between about 100-500 daltons. In one embodiment, a quaternary ammonium containing substance may have a molecular weight between about 100-400 daltons. In one embodiment, a quaternary ammonium containing substance may have a molecular weight between about 100-300 daltons. In one embodiment, a quaternary ammonium containing substance may have a molecular weight between about 100-250 daltons.

One embodiment of the present invention is the method whereby one or more quaternary ammonium containing substance may be combined, such as by mixing, with at least one or more RNA substance to produce a mixture comprising at least one or more quaternary ammonium containing substance and at least one or more RNA substance. As a non-limiting example one or more RNA substance may be mixed with one or more quaternary ammonium containing substance.

Another embodiment of the present invention is the method whereby one or more quaternary ammonium containing substance may be combined, such as by mixing, with at least one or more RNA substance to produce a mixture comprising at least one or more RNA substance and at least one or more quaternary ammonium containing substance at one or more of the RNA stabilizing substance concentrations within the stabilizing substance molar concentration range list or in the stabilizing substance molar concentration list or the stabilizing substance weight percent concentration list as described herein. These same methods may be used to combine one or more other substances, including, but not limited to one or more cellular uptake agents or one or more additional RNA stabilizing substances, with one or more RNA substances and one or more quaternary ammonium containing substances to produce a mixture comprising one or more RNA substance, one or more quaternary ammonium containing substance, and one or more cellular uptake agent or one or more additional RNA stabilizing substance.

One embodiment of the present invention is the method whereby one or more quaternary ammonium containing substance may be combined, such as by mixing, with at least one or more RNA substance to produce a composition comprising at least one or more RNA substance and at least one or more quaternary ammonium containing substance. Another embodiment of the present invention is the method whereby one or more quaternary ammonium containing substance may be combined, such as by mixing, with one or more RNA substance to produce a composition comprising at least one or more RNA substance and at least one or more quaternary ammonium containing substance at one or more of the RNA stabilizing substance concentrations within the stabilizing substance molar concentration range list or in the stabilizing substance molar concentration list or stabilizing substance weight percent concentration list as described herein. These same methods may be used to combine one or more other substances, including, but not limited to one or more cellular uptake agents or one or more additional RNA stabilizing substance, with one or more RNA substance and one or more quaternary ammonium containing substance to produce a composition comprising one or more RNA substance, one or more quaternary ammonium containing substance, and one or more cellular uptake agent or one or more additional RNA stabilizing substance.

In one embodiment a quaternary ammonium containing substance may be monomeric. In one embodiment a quaternary ammonium containing substance may be aliphatic. In one embodiment a quaternary ammonium containing substance may comprise a ring structure. In another embodiment a quaternary ammonium containing substance may comprise a heterocyclic ring structure. In one embodiment a quaternary ammonium containing substance may comprise an aromatic ring structure. In another embodiment a quaternary ammonium containing substance may comprise an aromatic heterocyclic ring structure.

In one embodiment a quaternary ammonium containing substance may comprise a cationic moiety at about physiologic pH. In one embodiment a quaternary ammonium containing substance may comprise an anionic moiety at about physiologic pH. In one embodiment a quaternary ammonium containing substance may comprise a zwitterion at about physiologic pH.

In one embodiment a quaternary ammonium containing substance may comprise one or more PIF as described herein.

In one embodiment a quaternary ammonium containing substance may be monomeric. In one embodiment a quaternary ammonium containing substance may be polymeric. In one embodiment a quaternary ammonium containing substance may comprise a polymer.

In one embodiment, one or more quaternary ammonium containing substance may be at least part of a polymer comprised of at least one or more quaternary ammonium containing substances.

In one embodiment one or more composition comprising one or more RNA substance and one or more quaternary ammonium containing substance may comprise one or more water concentrations in the water concentration list or in the water concentration range list. In one embodiment one or more composition comprising one or more RNA substance, one or more quaternary ammonium containing substance, and one or more additional substance, such as one or more cellular uptake agent or one or more additional RNA stabilizing substance as non-limiting examples, may comprise one or more water concentrations in the water concentration list or in the water concentration range list. In one embodiment one or more composition comprising one or more RNA substance and one or more quaternary ammonium containing substance may have a dielectric constant of one or more dielectric constant in the dielectric constant list or in the dielectric constant range list. In one embodiment one or more composition comprising one or more RNA substance, one or more quaternary ammonium containing substance, and one or more additional substance, such as one or more cellular uptake agent or one or more additional RNA stabilizing substance as non-limiting examples, may have a dielectric constant of one or more dielectric constant in the dielectric constant list or in the dielectric constant range list.

Embodiments of the present invention may include compositions comprising one or more quaternary ammonium containing substance used in a composition comprising one or more RNA substance, one or more quaternary ammonium containing substance, and one or more of the following substances: water, cellular uptake agents, aprotic substances, low dielectric substances, cyclic phosphate or metaphosphate containing substances, non-carbohydrate organic osmolyte substances, tertiary sulfonium containing substances, quaternary phosphonium containing substances, hydrotrope containing substances, surfactant containing substances, betaine containing substances, stabilizing monomers, stabilizing polymers, or supplemental RNA stabilizing substances. Other embodiments may include one or more composition comprising one or more quaternary ammonium containing substance and one or more RNA substance and may also comprise one or more cellular uptake agent or one or more additional RNA stabilizing substances. Other embodiments may include compositions comprising one or more quaternary ammonium containing substance, one or more RNA substance, and one or more additional substances, such as: one or more cellular uptake agent, or one or more additional RNA stabilizing substance, or one or more buffering agent, or one or more chelating agent, or one or more inorganic salt, or water, as non-limiting examples.

Embodiments of the present invention may include one or more compositions comprising one or more RNA substance and one or more quaternary ammonium containing substance, wherein a composition is not lyophilized.

In one embodiment one or more composition comprising one or more RNA substance and one or more quaternary ammonium containing substance may not be lyophilized. In one embodiment one or more composition comprising one or more RNA substance, one or more quaternary ammonium containing substance, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more quaternary ammonium containing substance, wherein a composition may be lyophilized.

In one embodiment one or more composition comprising one or more RNA substance and one or more quaternary ammonium containing substance may be lyophilized. In one embodiment one or more composition comprising one or more RNA substance, one or more quaternary ammonium containing substance, and one or more additional substance, such as one or more cellular uptake agent or one or more additional RNA stabilizing substance as non-limiting examples, may be lyophilized.

Embodiments of the present invention may include one or more compositions comprising one or more quaternary ammonium containing substance and one or more RNA substance described herein, wherein one or more composition described herein may have a melting of one or more melting point in the stabilizing composition melting point list.

In one embodiment one or more composition comprising one or more RNA substance and one or more quaternary ammonium containing substance may have a melting point of one or more melting point in the stabilizing composition melting point list. In one embodiment one or more composition comprising one or more RNA substance, one or more quaternary ammonium containing substance, and one or more additional substance, such as one or more cellular uptake agent or one or more additional RNA stabilizing substance as a non-limiting example, may have a melting point of one or more melting point in the stabilizing composition melting point list.

### Quaternary Phosphonium Substances:

The inventors have discovered that quaternary phosphonium containing substances may stabilize RNA substances.

The inventors have discovered that RNA stabilizing substances may comprise quaternary phosphonium containing substances. The inventors have also discovered that the stability of RNA substances may be enhanced in an RNA storage environment comprising a quaternary phosphonium containing substance. The inventors have also discovered that the stability of RNA substances may be enhanced in an RNA storage environment comprising a quaternary phosphonium containing substance and one or more additional RNA stabilizing substance, such as an aprotic substance.

The inventors have also discovered that the stability of RNA substances may be enhanced in compositions comprising an RNA substance and a quaternary phosphonium containing substance. The inventors have also discovered that the stability of RNA substances may be enhanced in compositions comprising an RNA substance, a quaternary phosphonium containing substance, and one or more additional RNA stabilizing substance, such as an aprotic substance.

In one embodiment of the present invention, one or more RNA stabilizing substance may comprise a quaternary phosphonium containing substance.

One embodiment of the present invention may include a composition comprised of one or more RNA substance and one or more quaternary phosphonium containing substance. Another embodiment of the present invention may include a composition comprised of one or more RNA substance, one or more quaternary phosphonium containing substance, and one or more additional RNA stabilizing substance, such as an aprotic substance.

One embodiment of the present invention may include a combination or mixture comprising one or more RNA substance and one or more quaternary phosphonium containing substance. Another embodiment of the present invention may include a combination or mixture comprising one or more RNA substance, one or more quaternary phosphonium containing substance, and one or more additional RNA stabilizing substance, such as an aprotic substance.

Embodiments of the present invention that comprise one or more RNA substance and one or more quaternary phosphonium containing substance may include combining, such as by mixing, one or more RNA substance with one or more substance that comprises at least one or more quaternary phosphonium containing substance.

Embodiments of the present invention that comprise one or more RNA substance, one or more quaternary phosphonium containing substance, and one or more additional RNA stabilizing substance, such as an aprotic substance, may include combining, such as by mixing, one or more RNA substance with one or more substance that comprises at least one or more quaternary phosphonium containing substance and one or more substance that comprises at least one or more RNA stabilizing substance, such as an aprotic substance.

An embodiment of the present invention may include compositions of materials that comprise one or more RNA substance and at least one or more quaternary phosphonium containing substance. The storage environment that improves the stability of RNA substances may comprise at least one or more vapor, liquid, powder, or solid quaternary phosphonium containing substance.

An embodiment of the present invention may include compositions of materials that comprise one or more RNA substance, one or more quaternary phosphonium containing substance, and one or more additional RNA stabilizing substance, such as an aprotic substance. The storage environment that improves the stability of RNA substances may comprise at least one or more vapor, liquid, powder, or solid quaternary phosphonium containing substance and may comprise at least one or more vapor, liquid, powder, or solid RNA stabilizing substance, such as an aprotic substance.

An embodiment of the present invention may include combinations of materials that comprise one or more RNA substance and at least one or more quaternary phosphonium containing substance. The storage environment that improves the stability of RNA substances may comprise at least one or more vapor, liquid, powder, or solid quaternary phosphonium containing substance.

An embodiment of the present invention may include combinations of materials that comprise one or more RNA substance, one or more quaternary phosphonium containing substance, and one or more additional RNA stabilizing substance, such as an aprotic substance. The storage environment that improves the stability of RNA substances may comprise at least one or more vapor, liquid, powder, or solid quaternary phosphonium containing substance and may comprise at least one or more vapor, liquid, powder, or solid RNA stabilizing substance, such as an aprotic substance.

In one embodiment of the present invention a composition comprised of one or more RNA substance and one or more quaternary phosphonium containing substance, produces a mixture with at least one or more RNA substance and at least one or more quaternary phosphonium containing substance. In one embodiment of the present invention a composition comprised of one or more RNA substance, one or more quaternary phosphonium containing substance, and one or more additional RNA stabilizing substance, such as an aprotic substance, produces a mixture with at least one or more RNA substance, at least one or more quaternary phosphonium containing substance, and at least one or more additional RNA stabilizing substance, such as an aprotic substance.

In one embodiment of the present invention a combination comprising one or more RNA substance and one or more quaternary phosphonium containing substance, produces a mixture with at least one or more RNA substance and at least one or more quaternary phosphonium containing substance. In one embodiment of the present invention a combination comprising one or more RNA substance, one or more quaternary phosphonium containing substance, and one or more additional RNA stabilizing substance, such as an aprotic substance, produces a mixture with at least one or more RNA substance, at least one or more quaternary phosphonium containing substance, and at least one or more additional RNA stabilizing substance, such as an aprotic substance.

In one embodiment of the present invention a composition with improved RNA stability may comprise one or more RNA substance and one or more quaternary phosphonium containing substance. In one embodiment of the present invention a composition with improved RNA stability may comprise one or more RNA substance, one or more quaternary phosphonium containing substance, and one or more additional RNA stabilizing substance, such as an aprotic substance.

In one embodiment of the present invention a composition with improved RNA stability may comprise a mixture of one or more RNA substance and one or more quaternary phosphonium containing substance. In one embodiment of the present invention a composition with improved RNA stability may comprise a mixture of one or more RNA substance, one or more quaternary phosphonium containing substance, and one or more additional RNA stabilizing substance, such as an aprotic substance.

In one embodiment of the present invention, each component included in a composition comprising one or more RNA substance and one or more quaternary phosphonium containing substance, may be stored separately, such as in a kit, or such as individual substances or as mixtures of one or more substance, and then combined later to produce a composition comprising one or more RNA substance and one or more quaternary phosphonium containing substance.

In one embodiment of the present invention, each component included in a composition comprising one or more RNA substance, one or more quaternary phosphonium containing substance, and one or more additional RNA stabilizing substance, such as an aprotic substance, may be stored separately, such as in a kit, or such as individually or as mixtures of one or more substance, and then combined later to produce a composition comprising one or more RNA substance, one or more quaternary phosphonium containing substance, and one or more additional RNA stabilizing substance, such as an aprotic substance.

In one embodiment of the present invention a composition comprising one or more RNA substance and one or more quaternary phosphonium containing substance may be at least partially biocompatible.

In one embodiment of the present invention a composition comprising one or more RNA substance, one or more quaternary phosphonium containing substance, and one or more additional RNA stabilizing substance, such as an aprotic substance, may be at least partially biocompatible.

In one embodiment of the present invention a combination comprised of one or more RNA substance and one or more quaternary phosphonium containing substance may be at least partially biocompatible.

In one embodiment of the present invention a combination comprised of one or more RNA substance, one or more quaternary phosphonium containing substance, and one or more additional RNA stabilizing substance, such as an aprotic substance, may be at least partially biocompatible.

Embodiments of the present invention comprising at least one or more quaternary phosphonium containing substances may include one or more forms of the substance. These forms may include, but are not limited to, one more counterions, ionic forms, conjugate bases, conjugate acids, protonated or deprotonated forms, hydrated or dehydrated forms, isomers, structural isomers, stereo isomers, chiral forms, salts, or combinations thereof.

In one embodiment a quaternary phosphonium containing substance may comprise one or more quaternary phosphonium cation. In one embodiment a quaternary phosphonium containing substance may comprise 1 or more quaternary phosphonium cations, 2 or more quaternary phosphonium cations, 3 or more quaternary phosphonium cations, 4 or more quaternary phosphonium cations, 5 or more quaternary phosphonium cations, 10 or more quaternary phosphonium cations, 50 or more quaternary phosphonium cations, or 100 or more quaternary phosphonium cations. In one embodiment a quaternary phosphonium containing substance may comprise between about 1-1,000 quaternary phosphonium cations, or between about 1-500 quaternary phosphonium cations, or between about 1-200 quaternary phosphonium cations, or between about 1-100 quaternary phosphonium cations, or between about 1-50 quaternary phosphonium cations, or between about 1-20 quaternary phosphonium cations, or between about 1-10 quaternary phosphonium cations, or between about 1-5 quaternary phosphonium cations, or between about 1-3 quaternary phosphonium cations, or between about 1-2 quaternary phosphonium cations, or between about 2-1,000 quaternary phosphonium cations, or between about 2-500 quaternary phosphonium cations, or between about 2-200 quaternary phosphonium cations, or between about 2-100 quaternary phosphonium cations, or between about 2-50 quaternary phosphonium cations, or between about 2-20 quaternary phosphonium cations, or between about 2-10 quaternary phosphonium cations, or between about 2-5 quaternary phosphonium cations, or between about 2-3 quaternary phosphonium cations.

In one embodiment a quaternary phosphonium containing substance may be hydroxylated. In one embodiment a quaternary phosphonium containing substance may comprise one or more hydroxy groups. In one embodiment a quaternary phosphonium containing substance may comprise 1 or more hydroxy groups, 2 or more hydroxy groups, 3 or more hydroxy groups, 4 or more hydroxy groups, 5 or more hydroxy groups, 10 or more hydroxy groups, or 50 or more hydroxy groups. In one embodiment a quaternary phosphonium containing substance may comprise between about 1-100 hydroxy groups, or between about 1-50 hydroxy groups, or between about 1-20 hydroxy groups, or between about 1-10 hydroxy groups, or between about 1-5 hydroxy groups, or between about 1-3 hydroxy groups, or between about 2-100 hydroxy groups, or between about 2-50 hydroxy groups, or between about 2-20 hydroxy groups, or between about 2-10 hydroxy groups, or between about 2-5 hydroxy groups, or between about 2-3 hydroxy groups.

In one embodiment a quaternary phosphonium containing substance may be carboxylated. In one embodiment a quaternary phosphonium containing substance may comprise one or more carboxylate or carboxylic acid groups. In one embodiment a quaternary phosphonium containing substance may comprise 1 or more carboxylate or carboxylic acid groups, 2 or more carboxylate or carboxylic acid groups, 3 or more carboxylate or carboxylic acid groups, 4 or more carboxylate or carboxylic acid groups, 5 or more carboxylate or carboxylic acid groups, 10 or more carboxylate or carboxylic acid groups, or 50 or more carboxylate or carboxylic acid groups. In one embodiment a quaternary phosphonium containing substance may comprise between about 1-100 carboxylate or carboxylic acid groups, or between about 1-50 carboxylate or carboxylic acid groups, or between about 1-20 carboxylate or carboxylic acid groups, or between about 1-10 carboxylate or carboxylic acid groups, or between about 1-5 carboxylate or carboxylic acid groups, or between about 1-3 carboxylate or carboxylic acid groups, or between about 2-100 carboxylate or carboxylic acid groups, or between about 2-50 carboxylate or carboxylic acid groups, or between about 2-20 carboxylate or carboxylic acid groups, or between about 2-10 carboxylate or carboxylic acid groups, or between about 2-5 carboxylate or carboxylic acid groups, or between about 2-3 carboxylate or carboxylic acid groups.

In one embodiment a quaternary phosphonium containing substance may comprise one or more ester bond. In one embodiment a quaternary phosphonium containing substance may comprise 1 or more ester bonds, 2 or more ester bonds, 3 or more ester bonds, 4 or more ester bonds, 5 or more ester bonds, 10 or more ester bonds, or 50 or more ester bonds. In one embodiment a quaternary phosphonium containing substance may comprise between about 1-100 ester bonds, or between about 1-50 ester bonds, or between about 1-20 ester bonds, or between about 1-10 ester bonds, or between about 1-5 ester bonds, or between about 1-3 ester bonds, or between about 2-100 ester bonds, or between about 2-50 ester bonds, or between about 2-20 ester bonds, or between about 2-10 ester bonds, or between about 2-5 ester bonds, or between about 2-3 ester bonds.

In one embodiment a quaternary phosphonium containing substance may comprise one or more ester, such as a carboxylate ester or carbonate ester as non-limiting examples. In one embodiment a quaternary phosphonium containing substance may comprise 1 or more esters, 2 or more esters, 3 or more esters, 4 or more esters, 5 or more esters, 10 or more esters, or 50 or more esters. In one embodiment a quaternary phosphonium containing substance may comprise between about 1-100 esters, or between about 1-50 esters, or between about 1-20 esters, or between about 1-10 esters, or between about 1-5 esters, or between about 1-3 esters, or between about 2-100 esters, or between about 2-50 esters, or between about 2-20 esters, or between about 2-10 esters, or between about 2-5 esters, or between about 2-3 esters.

In one embodiment a quaternary phosphonium containing substance may comprise one or more ether bond. In one embodiment a quaternary phosphonium containing substance may comprise 1 or more ether bonds, 2 or more ether bonds, 3 or more ether bonds, 4 or more ether bonds, 5 or more ether bonds, 10 or more ether bonds, or 50 or more ether bonds. In one embodiment a quaternary phosphonium containing substance may comprise between about 1-100 ether bonds, or between about 1-50 ether bonds, or between about 1-20 ether bonds, or between about 1-10 ether bonds, or between about 1-5 ether bonds, or between about 1-3 ether bonds, or between about 2-100 ether bonds, or between about 2-50 ether bonds, or between about 2-20 ether bonds, or between about 2-10 ether bonds, or between about 2-5 ether bonds, or between about 2-3 ether bonds.

In one embodiment a quaternary phosphonium containing substance may comprise one or more ether. In one embodiment a quaternary phosphonium containing substance may comprise 1 or more ethers, 2 or more ethers, 3 or more ethers, 4 or more ethers, 5 or more ethers, 10 or more ethers, or 50 or more ethers. In one embodiment a quaternary phosphonium containing substance may comprise between about 1-100 ethers, or between about 1-50 ethers, or between about 1-20 ethers, or between about 1-10 ethers, or between about 1-5 ethers, or between about 1-3 ethers, or between about 2-100 ethers, or between about 2-50 ethers, or between about 2-20 ethers, or between about 2-10 ethers, or between about 2-5 ethers, or between about 2-3 ethers.

In one embodiment a quaternary phosphonium containing substance may comprise one or more amide bond. In one embodiment a quaternary phosphonium containing substance may comprise 1 or more amide bonds, 2 or more amide bonds, 3 or more amide bonds, 4 or more amide bonds, 5 or more amide bonds, 10 or more amide bonds, or 50 or more amide bonds. In one embodiment a quaternary phosphonium containing substance may comprise between about 1-100 amide bonds, or between about 1-50 amide bonds, or between about 1-20 amide bonds, or between about 1-10 amide bonds, or between about 1-5 amide bonds, or between about 1-3 amide bonds, or between about 2-100 amide bonds, or between about 2-50 amide bonds, or between about 2-20 amide bonds, or between about 2-10 amide bonds, or between about 2-5 amide bonds, or between about 2-3 amide bonds.

In one embodiment a quaternary phosphonium containing substance may comprise one or more amide group. In one embodiment a quaternary phosphonium containing substance may comprise 1 or more amide groups, 2 or more amide groups, 3 or more amide groups, 4 or more amide groups, 5 or more amide groups, 10 or more amide groups, or 50 or more amide groups. In one embodiment a quaternary phosphonium containing substance may comprise between about 1-100 amide groups, or between about 1-50 amide groups, or between about 1-20 amide groups, or between about 1-10 amide groups, or between about 1-5 amide groups, or between about 1-3 amide groups, or between about 2-100 amide groups, or between about 2-50 amide groups, or between about 2-20 amide groups, or between about 2-10 amide groups, or between about 2-5 amide groups, or between about 2-3 amide groups.

In one embodiment a quaternary phosphonium containing substance may comprise one or more organosulfate group. In one embodiment a quaternary phosphonium containing substance may comprise 1 or more organosulfate groups, 2 or more organosulfate groups, 3 or more organosulfate groups, 4 or more organosulfate groups, 5 or more organosulfate groups, 10 or more organosulfate groups, or 50 or more organosulfate groups. In one embodiment a quaternary phosphonium containing substance may comprise between about 1-100 organosulfate groups, or between about 1-50 organosulfate groups, or between about 1-20 organosulfate groups, or between about 1-10 organosulfate groups, or between about 1-5 organosulfate groups, or between about 1-3 organosulfate groups, or between about 2-100 organosulfate groups, or between about 2-50 organosulfate groups, or between about 2-20 organosulfate groups, or between about 2-10 organosulfate groups, or between about 2-5 organosulfate groups, or between about 2-3 organosulfate groups.

In one embodiment a quaternary phosphonium containing substance may comprise one or more sulfonate group. In one embodiment a quaternary phosphonium containing substance may comprise 1 or more sulfonate groups, 2 or more sulfonate groups, 3 or more sulfonate groups, 4 or more sulfonate groups, 5 or more sulfonate groups, 10 or more sulfonate groups, 50 or more sulfonate groups. In one embodiment a quaternary phosphonium containing substance may comprise between about 1-100 sulfonate groups, or between about 1-50 sulfonate groups, or between about 1-20 sulfonate groups, or between about 1-10 sulfonate groups, or between about 1-5 sulfonate groups, or between about 1-3 sulfonate groups, or between about 2-100 sulfonate groups, or between about 2-50 sulfonate groups, or between about 2-20 sulfonate groups, or between about 2-10 sulfonate groups, or between about 2-5 sulfonate groups, or between about 2-3 sulfonate groups.

In one embodiment a quaternary phosphonium containing substance may comprise one or more organophosphate group. In one embodiment a quaternary phosphonium containing substance may comprise 1 or more organophosphate groups, 2 or more organophosphate groups, 3 or more organophosphate groups, 4 or more organophosphate groups, 5 or more organophosphate groups, 10 or more organophosphate groups, or 50 or more organophosphate groups. In one embodiment a quaternary phosphonium containing substance may comprise between about 1-100 organophosphate groups, or between about 1-50 organophosphate groups, or between about 1-20 organophosphate groups, or between about 1-10 organophosphate groups, or between about 1-5 organophosphate groups, or between about 1-3 organophosphate groups, or between about 2-100 organophosphate groups, or between about 2-50 organophosphate groups, or between about 2-20 organophosphate groups, or between about 2-10 organophosphate groups, or between about 2-5 organophosphate groups, or between about 2-5 organophosphate groups, or between about 2-3 organophosphate groups.

In one embodiment a quaternary phosphonium containing substance may comprise one or more aryl group. In one embodiment a quaternary phosphonium containing substance may comprise 1 or more aryl groups, 2 or more aryl groups, 3 or more aryl groups, 4 or more aryl groups, 5 or more aryl groups, 10 or more aryl groups, or 50 or more aryl groups. In one embodiment a quaternary phosphonium containing substance may comprise between about 1-100 aryl groups, or between about 1-50 aryl groups, or between about 1-20 aryl groups, or between about 1-10 aryl groups, or between about 1-5 aryl groups, or between about 1-3 aryl groups, or between about 2-100 aryl groups, or between about 2-50 aryl groups, or between about 2-20 aryl groups, or between about 2-10 aryl groups, or between about 2-5 aryl groups, or between about 2-3 aryl groups.

In one embodiment a quaternary phosphonium containing substance may not comprise a hydroxy group. In one embodiment a quaternary phosphonium containing substance may not comprise a carboxylate or carboxylic acid group. In one embodiment a quaternary phosphonium containing substance may not comprise an ester bond. In one embodiment a quaternary phosphonium containing substance may not comprise an ester. In one embodiment a quaternary phosphonium containing substance may not comprise an ether. In one embodiment a quaternary phosphonium containing substance may not comprise an ether bond. In one embodiment a quaternary phosphonium containing substance may not comprise an organosulfate group. In one embodiment a quaternary phosphonium containing substance may not comprise a sulfonate group. In one embodiment a quaternary phosphonium containing substance may not comprise an organophosphate group.

In one embodiment a quaternary phosphonium containing substance may comprise one or more polymer. In one embodiment a quaternary phosphonium containing substance may be polymeric. In one embodiment a quaternary phosphonium containing substance may comprise one or more cation. In one embodiment a quaternary phosphonium containing substance may be cationic. In one embodiment a quaternary phosphonium containing substance may be polycationic. In one embodiment a quaternary phosphonium containing substance may comprise one or more anion. In one embodiment a quaternary phosphonium containing substance may comprise one or more zwitterion. In one embodiment a quaternary phosphonium containing substance may be zwitterionic.

In one embodiment a quaternary phosphonium containing substance may be aliphatic.

In one embodiment a quaternary phosphonium containing substance may comprise one or more ring structure. In one embodiment a quaternary phosphonium containing substance may comprise one or more aromatic ring structure. In one embodiment a quaternary phosphonium containing substance may comprise one or more six membered ring structure. In one embodiment a quaternary phosphonium containing substance may comprise one or more aromatic six membered ring structure.

In one embodiment a quaternary phosphonium containing substance may not be polymeric. In one embodiment a quaternary phosphonium containing substance may not be polycationic. In one embodiment a quaternary phosphonium containing substance may not comprise an anion. In one embodiment a quaternary phosphonium containing substance may not comprise a zwitterion. In one embodiment a quaternary phosphonium containing substance may not be zwitterionic.

In one embodiment a quaternary phosphonium containing substance may not comprise an aromatic ring structure. In one embodiment a quaternary phosphonium containing substance may not comprise a six membered ring structure.

A non-limiting example of a quaternary phosphonium containing substance comprised of one or more quaternary phosphonium that may be used is tributylhexadecylphosphonium, wherein tributylhexadecylphosphonium may be substituted for or used in combination with one or more quaternary phosphonium containing substances described herein. One embodiment of the present invention may be a combination of substances comprising tributylhexadecylphosphonium and another embodiment may also comprise at least one RNA substance. Other embodiments may be combinations of substances comprising tributylhexadecylphosphonium wherein the concentration of tributylhexadecylphosphonium may be one or more concentration in the stabilizing substance weight percent concentration list or in the stabilizing substance molar concentration range list or in the stabilizing substance molar concentration list.

A non-limiting example of a quaternary phosphonium containing substance comprised of one or more quaternary phosphonium that may be used is 3-(triphenylphosphonio)propane-1-sulfonate (TPPS), wherein TPPS may be substituted for or used in combination with one or more quaternary phosphonium containing substances described herein. One embodiment of the present invention may be a combination of substances comprising TPPS and another embodiment may also comprise at least one RNA substance. Other embodiments may be combinations of substances comprising TPPS wherein the concentration of TPPS may be one or more concentration in the stabilizing substance weight percent concentration list or in the stabilizing substance molar concentration range list or in the stabilizing substance molar concentration list.

A non-limiting example of a quaternary phosphonium containing substance comprised of one or more quaternary phosphonium that may be used is 3-(di-tert-butylphosphonium)propane-1-sulfonate, wherein 3-(di-tert-butylphosphonium)propane-1-sulfonate may be substituted for or used in combination with one or more quaternary phosphonium containing substances described herein. One embodiment of the present invention may be a combination of substances comprising 3-(di-tert-butylphosphonium)propane-1-sulfonate and another embodiment may also comprise at least one RNA substance. Other embodiments may be combinations of substances comprising 3-(di-tert-butylphosphonium)propane-1-sulfonate wherein the concentration of 3-(di-tert-butylphosphonium)propane-1-sulfonate may be one or more concentration in the stabilizing substance weight percent concentration list or in the stabilizing substance molar concentration range list or in the stabilizing substance molar concentration list.

A non-limiting example of a quaternary phosphonium containing substance comprised of one or more quaternary phosphonium that may be used is 4-(triphenylphosphonio)butane-1-sulfonate (TPBS), wherein TPBS may be substituted for or used in combination with one or more quaternary phosphonium containing substances described herein. One embodiment of the present invention may be a combination of substances comprising TPBS and another embodiment may also comprise at least one RNA substance. Other embodiments may be combinations of substances comprising TPBS wherein the concentration of TPBS may be one or more concentration in the stabilizing substance weight percent concentration list or in the stabilizing substance molar concentration range list or in the stabilizing substance molar concentration list.

For conciseness, the following list of quaternary phosphonium containing substances, is herein referred to as the quaternary phosphonium containing substance list wherein one or more of the following substances may be substituted for or used in combination with one or more quaternary phosphonium containing substance as described herein.

In other embodiments, other quaternary phosphonium containing substances that may be substituted for or used in combination with one or more quaternary phosphonium containing substance as described herein may comprise one or more of the following, including but not limited to: tributylhexadecylphosphonium, TPPS, TPBS, 3-(di-tert-butylphosphonium)propane-1-sulfonate, (methoxycarbonylmethyl)triphenylphosphonium, acetonyltriphenylphosphonium, 4-(triphenylphosphonio)butane-1-sulfonate, 5-(carboxypentyl)(triphenyl)phosphonium, 3-(triethylphosphonio)propane-1-sulfonate, 3-(tributylphosphonio)propane-1-sulfonate, 3-(tri-tert-butylphosphonio)propane-1-sulfonate, 3-(trimethylphosphonio)propane-1-sulfonate, 3-(tripropylphosphonio)propane-1-sulfonate, phosphonium sulfonates, phosphonium sulfates, phosphonium carboxylates, phosphonium phosphates, and phosphonium phosphonates, or derivatives, or combinations thereof.

Embodiments of the present invention may include combinations comprising one or more of the substances from the quaternary phosphonium containing substance list substituted for or used in combination with one or more quaternary phosphonium containing substance or one or more RNA stabilizing substance, such as an aprotic substance as a non-limiting example, and other embodiments may also comprise at least one RNA substance.

Other embodiments may include combinations of substances comprising one or more quaternary phosphonium containing substance selected from the quaternary phosphonium containing substance list wherein the concentration of one or more substances may be one or more concentration in the stabilizing substance weight percent concentration list or in the stabilizing substance molar concentration range list or in the stabilizing substance molar concentration list.

In one embodiment, a quaternary phosphonium containing substance may have a molecular weight between about 100-1,000,000,000 daltons. In one embodiment, a quaternary phosphonium containing substance may have a molecular weight between about 100-100,000,000 daltons. In one embodiment, a quaternary phosphonium containing substance may have a molecular weight between about 100-10,000,000 daltons. In one embodiment, a quaternary phosphonium containing substance may have a molecular weight between about 100-1,000,000 daltons. In one embodiment, a quaternary phosphonium containing substance may have a molecular weight between about 100-500,000 daltons. In one embodiment, a quaternary phosphonium containing substance may have a molecular weight between about 100-200,000 daltons. In one embodiment, a quaternary phosphonium containing substance may have a molecular weight between about 100-100,000 daltons. In one embodiment, a quaternary phosphonium containing substance may have a molecular weight between about 100-50,000 daltons. In one embodiment, a quaternary phosphonium containing substance may have a molecular weight between about 100-20,000 daltons. In one embodiment, a quaternary phosphonium containing substance may have a molecular weight between about 100-10,000 daltons. In one embodiment, a quaternary phosphonium containing substance may have a molecular weight between about 100-5,000 daltons. In one embodiment, a quaternary phosphonium containing substance may have a molecular weight between about 100-2,000 daltons. In one embodiment, a quaternary phosphonium containing substance may have a molecular weight between about 100-1,000 daltons. In one embodiment, a quaternary phosphonium containing substance may have a molecular weight between about 100-900 daltons. In one embodiment, a quaternary phosphonium containing substance may have a molecular weight between about 100-800 daltons. In one embodiment, a quaternary phosphonium containing substance may have a molecular weight between about 100-700 daltons. In one embodiment, a quaternary phosphonium containing substance may have a molecular weight between about 100-600 daltons. In one embodiment, a quaternary phosphonium containing substance may have a molecular weight between about 100-500 daltons. In one embodiment, a quaternary phosphonium containing substance may have a molecular weight between about 100-400 daltons. In one embodiment, a quaternary phosphonium containing substance may have a molecular weight between about 100-300 daltons.

In one embodiment, a quaternary phosphonium containing substance may have a molecular weight between about 150-1,000,000,000 daltons. In one embodiment, a quaternary phosphonium containing substance may have a molecular weight between about 150-100,000,000 daltons. In one embodiment, a quaternary phosphonium containing substance may have a molecular weight between about 150-10,000,000 daltons. In one embodiment, a quaternary phosphonium containing substance may have a molecular weight between about 150-1,000,000 daltons. In one embodiment, a quaternary phosphonium containing substance may have a molecular weight between about 150-500,000 daltons. In one embodiment, a quaternary phosphonium containing substance may have a molecular weight between about 150-200,000 daltons. In one embodiment, a quaternary phosphonium containing substance may have a molecular weight between about 150-100,000 daltons. In one embodiment, a quaternary phosphonium containing substance may have a molecular weight between about 150-50,000 daltons. In one embodiment, a quaternary phosphonium containing substance may have a molecular weight between about 150-20,000 daltons. In one embodiment, a quaternary phosphonium containing substance may have a molecular weight between about 150-10,000 daltons. In one embodiment, a quaternary phosphonium containing substance may have a molecular weight between about 150-5,000 daltons. In one embodiment, a quaternary phosphonium containing substance may have a molecular weight between about 150-2,000 daltons. In one embodiment, a quaternary phosphonium containing substance may have a molecular weight between about 150-1,000 daltons. In one embodiment, a quaternary phosphonium containing substance may have a molecular weight between about 150-900 daltons. In one embodiment, a quaternary phosphonium containing substance may have a molecular weight between about 150-800 daltons. In one embodiment, a quaternary phosphonium containing substance may have a molecular weight between about 150-700 daltons. In one embodiment, a quaternary phosphonium containing substance may have a molecular weight between about 150-600 daltons. In one embodiment, a quaternary phosphonium containing substance may have a molecular weight between about 150-500 daltons. In one embodiment, a quaternary phosphonium containing substance may have a molecular weight between about 150-400 daltons. In one embodiment, a quaternary phosphonium containing substance may have a molecular weight between about 150-300 daltons.

One embodiment of the present invention is the method whereby one or more quaternary phosphonium containing substance may be combined, such as by mixing, with at least one or more RNA substance to produce a mixture comprising at least one or more quaternary phosphonium containing substance and at least one or more RNA substance. As a non-limiting example one or more RNA substance may be mixed with one or more quaternary phosphonium containing substance.

Another embodiment of the present invention is the method whereby one or more quaternary phosphonium containing substance may be combined, such as by mixing, with at least one or more RNA substance to produce a mixture comprising at least one or more RNA substance and at least one or more quaternary phosphonium containing substance at one or more of the RNA stabilizing substance concentrations within the stabilizing substance molar concentration range list or in the stabilizing substance molar concentration list or the stabilizing substance weight percent concentration list as described herein. These same methods may be used to combine one or more other substances, including, but not limited to one or more cellular uptake agents or one or more additional RNA stabilizing substances, with one or more RNA substances and one or more quaternary phosphonium containing substances to produce a mixture comprising one or more RNA substance, one or more quaternary phosphonium containing substance, and one or more cellular uptake agent or one or more additional RNA stabilizing substance.

One embodiment of the present invention is the method whereby one or more quaternary phosphonium containing substance may be combined, such as by mixing, with at least one or more RNA substance to produce a composition comprising at least one or more RNA substance and at least one or more quaternary phosphonium containing substance. Another embodiment of the present invention is the method whereby one or more quaternary phosphonium containing substance may be combined, such as by mixing, with one or more RNA substance to produce a composition comprising at least one or more RNA substance and at least one or more quaternary phosphonium containing substance at one or more of the RNA stabilizing substance concentrations within the stabilizing substance molar concentration range list or in the stabilizing substance molar concentration list or stabilizing substance weight percent concentration list as described herein. These same methods may be used to combine one or more other substances, including, but not limited to one or more cellular uptake agents or one or more additional RNA stabilizing substance, with one or more RNA substance and one or more quaternary phosphonium containing substance to produce a composition comprising one or more RNA substance, one or more quaternary phosphonium containing substance, and one or more cellular uptake agent or one or more additional RNA stabilizing substance.

In one embodiment a quaternary phosphonium containing substance may be monomeric. In one embodiment a quaternary phosphonium containing substance may be aliphatic. In one embodiment a quaternary phosphonium containing substance may comprise a ring structure. In another embodiment a quaternary phosphonium containing substance may comprise a heterocyclic ring structure. In one embodiment a quaternary phosphonium containing substance may comprise an aromatic ring structure. In another embodiment a quaternary phosphonium containing substance may comprise an aromatic heterocyclic ring structure.

In one embodiment a quaternary phosphonium containing substance may comprise a cationic moiety at about physiologic pH. In one embodiment a quaternary phosphonium containing substance may comprise an anionic moiety at about physiologic pH. In one embodiment a quaternary phosphonium containing substance may comprise a zwitterion at about physiologic pH. In one embodiment a quaternary phosphonium containing substance may be zwitterionic at about physiologic pH.

In one embodiment a quaternary phosphonium containing substance may comprise one or more PIF as described herein.

In one embodiment a quaternary phosphonium containing substance may be monomeric. In one embodiment a quaternary phosphonium containing substance may be polymeric. In one embodiment a quaternary phosphonium containing substance may comprise a polymer.

In one embodiment, one or more quaternary phosphonium containing substance may be at least part of a polymer comprised of at least one or more quaternary phosphonium containing substances.

In one embodiment one or more composition comprising one or more RNA substance and one or more quaternary phosphonium containing substance may comprise one or more water concentrations in the water concentration list or in the water concentration range list. In one embodiment one or more composition comprising one or more RNA substance, one or more quaternary phosphonium containing substance, and one or more additional substance, such as one or more cellular uptake agent or one or more additional RNA stabilizing substance as non-limiting examples, may comprise one or more water concentrations in the water concentration list or in the water concentration range list. In one embodiment one or more composition comprising one or more RNA substance and one or more quaternary phosphonium containing substance may have a dielectric constant of one or more dielectric constant in the dielectric constant list or in the dielectric constant range list. In one embodiment one or more composition comprising one or more RNA substance, one or more quaternary phosphonium containing substance, and one or more additional substance, such as one or more cellular uptake agent or one or more additional RNA stabilizing substance as non-limiting examples, may have a dielectric constant of one or more dielectric constant in the dielectric constant list or in the dielectric constant range list.

Embodiments of the present invention may include compositions comprising one or more quaternary phosphonium containing substance used in a composition comprising one or more RNA substance, one or more quaternary phosphonium containing substance, and one or more of the following substances: water, cellular uptake agents, aprotic substances, low dielectric substances, cyclic phosphate or metaphosphate containing substances, non-carbohydrate organic osmolyte substances, tertiary sulfonium containing substances, quaternary ammonium containing substances, hydrotrope containing substances, surfactant containing substances, betaine containing substances, stabilizing monomers, stabilizing polymers, or supplemental RNA stabilizing substances. Other embodiments may include one or more composition comprising one or more quaternary phosphonium containing substance and one or more RNA substance and may also comprise one or more cellular uptake agent or one or more additional RNA stabilizing substances. Other embodiments may include compositions comprising one or more quaternary phosphonium containing substance, one or more RNA substance, and one or more additional substances, such as: one or more cellular uptake agent, or one or more additional RNA stabilizing substance, or one or more buffering agent, or one or more chelating agent, or one or more inorganic salt, or water, as non-limiting examples.

Embodiments of the present invention may include one or more compositions comprising one or more RNA substance and one or more quaternary phosphonium containing substance, wherein a composition is not lyophilized.

In one embodiment one or more composition comprising one or more RNA substance and one or more quaternary phosphonium containing substance may not be lyophilized. In one embodiment one or more composition comprising one or more RNA substance, one or more quaternary phosphonium containing substance, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more quaternary phosphonium containing substance, wherein a composition may be lyophilized.

In one embodiment one or more composition comprising one or more RNA substance and one or more quaternary phosphonium containing substance may be lyophilized. In one embodiment one or more composition comprising one or more RNA substance, one or more quaternary phosphonium containing substance, and one or more additional substance, such as one or more cellular uptake agent or one or more additional RNA stabilizing substance as non-limiting examples, may be lyophilized.

Embodiments of the present invention may include one or more compositions comprising one or more quaternary phosphonium containing substance and one or more RNA substance described herein, wherein one or more composition described herein may have a melting of one or more melting point in the stabilizing composition melting point list.

In one embodiment one or more composition comprising one or more RNA substance and one or more quaternary phosphonium containing substance may have a melting point of one or more melting point in the stabilizing composition melting point list. In one embodiment one or more composition comprising one or more RNA substance, one or more quaternary phosphonium containing substance, and one or more additional substance, such as one or more cellular uptake agent or one or more additional RNA stabilizing substance as a non-limiting example, may have a melting point of one or more melting point in the stabilizing composition melting point list.

### Hydrotrope Containing Substances:

The inventors have discovered that hydrotrope containing substances may stabilize RNA substances.

The inventors have discovered that RNA stabilizing substances may comprise hydrotrope containing substances. The inventors have also discovered that the stability of RNA substances may be enhanced in an RNA storage environment comprising a hydrotrope containing substance. The inventors have also discovered that the stability of RNA substances may be enhanced in an RNA storage environment comprising a hydrotrope containing substance and one or more additional RNA stabilizing substance, such as an aprotic substance.

The inventors have also discovered that the stability of RNA substances may be enhanced in compositions comprising an RNA substance and a hydrotrope containing substance. The inventors have also discovered that the stability of RNA substances may be enhanced in compositions comprising an RNA substance, a hydrotrope containing substance, and one or more additional RNA stabilizing substance, such as an aprotic substance.

In one embodiment of the present invention, one or more RNA stabilizing substance may comprise a hydrotrope containing substance.

One embodiment of the present invention may include a composition comprised of one or more RNA substance and one or more hydrotrope containing substance. Another embodiment of the present invention may include a composition comprised of one or more RNA substance, one or more hydrotrope containing substance, and one or more additional RNA stabilizing substance, such as an aprotic substance.

One embodiment of the present invention may include a combination or mixture comprising one or more RNA substance and one or more hydrotrope containing substance. Another embodiment of the present invention may include a combination or mixture comprising one or more RNA substance, one or more hydrotrope containing substance, and one or more additional RNA stabilizing substance, such as an aprotic substance.

Embodiments of the present invention that comprise one or more RNA substance and one or more hydrotrope containing substance may include combining, such as by mixing, one or more RNA substance with one or more substance that comprises at least one or more hydrotrope containing substance.

Embodiments of the present invention that comprise one or more RNA substance, one or more hydrotrope containing substance, and one or more additional RNA stabilizing substance, such as an aprotic substance, may include combining, such as by mixing, one or more RNA substance with one or more substance that comprises at least one or more hydrotrope containing substance and one or more substance that comprises at least one or more RNA stabilizing substance, such as an aprotic substance.

An embodiment of the present invention may include compositions of materials that comprise one or more RNA substance and at least one or more hydrotrope containing substance. The storage environment that improves the stability of RNA substances may comprise at least one or more vapor, liquid, powder, or solid hydrotrope containing substance.

An embodiment of the present invention may include compositions of materials that comprise one or more RNA substance, one or more hydrotrope containing substance, and one or more additional RNA stabilizing substance, such as an aprotic substance. The storage environment that improves the stability of RNA substances may comprise at least one or more vapor, liquid, powder, or solid hydrotrope containing substance and may comprise at least one or more vapor, liquid, powder, or solid RNA stabilizing substance, such as an aprotic substance.

An embodiment of the present invention may include combinations of materials that comprise one or more RNA substance and at least one or more hydrotrope containing substance. The storage environment that improves the stability of RNA substances may comprise at least one or more vapor, liquid, powder, or solid hydrotrope containing substance.

An embodiment of the present invention may include combinations of materials that comprise one or more RNA substance, one or more hydrotrope containing substance, and one or more additional RNA stabilizing substance, such as an aprotic substance. The storage environment that improves the stability of RNA substances may comprise at least one or more vapor, liquid, powder, or solid hydrotrope containing substance and may comprise at least one or more vapor, liquid, powder, or solid RNA stabilizing substance, such as an aprotic substance.

In one embodiment of the present invention a composition comprised of one or more RNA substance and one or more hydrotrope containing substance, produces a mixture with at least one or more RNA substance and at least one or more hydrotrope containing substance. In one embodiment of the present invention a composition comprised of one or more RNA substance, one or more hydrotrope containing substance, and one or more additional RNA stabilizing substance, such as an aprotic substance, produces a mixture with at least one or more RNA substance, at least one or more hydrotrope containing substance, and at least one or more additional RNA stabilizing substance, such as an aprotic substance.

In one embodiment of the present invention a combination comprising one or more RNA substance and one or more hydrotrope containing substance, produces a mixture with at least one or more RNA substance and at least one or more hydrotrope containing substance. In one embodiment of the present invention a combination comprising one or more RNA substance, one or more hydrotrope containing substance, and one or more additional RNA stabilizing substance, such as an aprotic substance, produces a mixture with at least one or more RNA substance, at least one or more hydrotrope containing substance, and at least one or more additional RNA stabilizing substance, such as an aprotic substance.

In one embodiment of the present invention a composition with improved RNA stability may comprise one or more RNA substance and one or more hydrotrope containing substance. In one embodiment of the present invention a composition with improved RNA stability may comprise one or more RNA substance, one or more hydrotrope containing substance, and one or more additional RNA stabilizing substance, such as an aprotic substance.

In one embodiment of the present invention a composition with improved RNA stability may comprise a mixture of one or more RNA substance and one or more hydrotrope containing substance. In one embodiment of the present invention a composition with improved RNA stability may comprise a mixture of one or more RNA substance, one or more hydrotrope containing substance, and one or more additional RNA stabilizing substance, such as an aprotic substance.

In one embodiment of the present invention, each component included in a composition comprising one or more RNA substance and one or more hydrotrope containing substance, may be stored separately, such as in a kit, or such as individual substances or as mixtures of one or more substance, and then combined later to produce a composition comprising one or more RNA substance and one or more hydrotrope containing substance.

In one embodiment of the present invention, each component included in a composition comprising one or more RNA substance, one or more hydrotrope containing substance, and one or more additional RNA stabilizing substance, such as an aprotic substance, may be stored separately, such as in a kit, or such as individually or as mixtures of one or more substance, and then combined later to produce a composition comprising one or more RNA substance, one or more hydrotrope containing substance, and one or more additional RNA stabilizing substance, such as an aprotic substance.

In one embodiment of the present invention a composition comprising one or more RNA substance and one or more hydrotrope containing substance may be at least partially biocompatible.

In one embodiment of the present invention a composition comprising one or more RNA substance, one or more hydrotrope containing substance, and one or more additional RNA stabilizing substance, such as an aprotic substance, may be at least partially biocompatible.

In one embodiment of the present invention a combination comprised of one or more RNA substance and one or more hydrotrope containing substance may be at least partially biocompatible.

In one embodiment of the present invention a combination comprised of one or more RNA substance, one or more hydrotrope containing substance, and one or more additional RNA stabilizing substance, such as an aprotic substance, may be at least partially biocompatible.

Embodiments of the present invention comprising at least one or more hydrotrope containing substances may include one or more forms of the substance. These forms may include, but are not limited to, one more counterions, ionic forms, conjugate bases, conjugate acids, protonated or deprotonated forms, hydrated or dehydrated forms, isomers, structural isomers, stereo isomers, chiral forms, salts, or combinations thereof.

In one embodiment a hydrotrope containing substance may comprise an anionic moiety. In one embodiment a hydrotrope containing substance may comprise a cationic moiety. In one embodiment a hydrotrope containing substance may comprise one or more anionic moiety and one or more cationic moiety. In one embodiment a hydrotrope containing substance may be zwitterionic.

In one embodiment a hydrotrope containing substance may comprise one or more amide group. In one embodiment a hydrotrope containing substance may be comprised of one or more carboxylic acid or carboxylate group, or one or more organophosphate group, or one or more organosulfate group, or one or more sulfonic acid or sulfonate group, or one or more phosphonic acid or phosphonate group.

In one embodiment a hydrotrope containing substance may comprise a cyclic ring structure. In one embodiment a hydrotrope containing substance may comprise an aromatic cyclic ring structure. In one embodiment a hydrotrope containing substance may comprise a heterocyclic ring structure. In one embodiment a hydrotrope containing substance may comprise an aromatic heterocyclic ring structure. In one embodiment a hydrotrope containing substance may comprise a nitrogen heterocycle. In one embodiment a hydrotrope containing substance may comprise an aromatic nitrogen heterocycle.

In one embodiment a hydrotrope containing substance may comprise a five membered ring structure. In one embodiment a hydrotrope containing substance may comprise an aromatic five membered ring structure. In one embodiment a hydrotrope containing substance may comprise a heterocyclic five membered ring structure. In one embodiment a hydrotrope containing substance may comprise an aromatic heterocyclic five membered ring structure. In one embodiment a hydrotrope containing substance may comprise a five membered nitrogen heterocycle. In one embodiment a hydrotrope containing substance may comprise an aromatic five membered nitrogen heterocycle.

In one embodiment a hydrotrope containing substance may comprise a six membered ring structure. In one embodiment a hydrotrope containing substance may comprise an aromatic six membered ring structure. In one embodiment a hydrotrope containing substance may comprise a heterocyclic six membered ring structure. In one embodiment a hydrotrope containing substance may comprise an aromatic heterocyclic six membered ring structure. In one embodiment a hydrotrope containing substance may comprise a six membered nitrogen heterocycle. In one embodiment a hydrotrope containing substance may comprise an aromatic six membered nitrogen heterocycle.

In one embodiment a hydrotrope containing substance may comprise an aryl group. In one embodiment a hydrotrope containing substance may comprise a phenyl group. In one embodiment a hydrotrope containing substance may comprise a benzene ring.

In one embodiment a hydrotrope containing substance may comprise a phenol, wherein a phenol is a phenyl group that may be bonded to one or more hydroxy groups, two more hydroxy groups, three or more hydroxy groups, four or more hydroxy groups or five or more hydroxy groups, or between about 1-6 hydroxy groups, or between about 1-5 hydroxy groups, or between about 1-4 hydroxy groups, or between about 1-3 hydroxy groups, or between about 1-2 hydroxy groups. In one embodiment a hydrotrope containing substance may comprise a polyphenol, wherein a polyphenol may comprise two or more phenol groups, three or more phenol groups, four or more phenol groups, five or more phenol groups, six or more phenol groups, seven or more phenol groups, eight or more phenol groups, nine or more phenol groups, or ten or more phenol groups. In one embodiment a hydrotrope containing substance may comprise an aryloxy group. In one embodiment a hydrotrope containing substance may comprise an alkoxy group.

In one embodiment a hydrotrope containing substance may comprise a pyrrole group. In one embodiment a hydrotrope containing substance may comprise a pyrroline group. In one embodiment a hydrotrope containing substance may comprise pyrrolidine group. In one embodiment a hydrotrope containing substance may comprise pyrrolidinium group. In one embodiment a hydrotrope containing substance may comprise an indole or indolium group. In one embodiment a hydrotrope containing substance may comprise a pyrrolizidine group. In one embodiment a hydrotrope containing substance may comprise a piperidine or piperidinium group. In one embodiment a hydrotrope containing substance may comprise a quinoline group. In one embodiment a hydrotrope containing substance may comprise an isoquinoline group. In one embodiment a hydrotrope containing substance may comprise a quinolizidine group. In one embodiment a hydrotrope containing substance may comprise an indolizidine group. In one embodiment a hydrotrope containing substance may comprise an oxazole group. In one embodiment a hydrotrope containing substance may comprise an isoxazole group. In one embodiment a hydrotrope containing substance may comprise a thiazole group. In one embodiment a hydrotrope containing substance may comprise an acridine group. In one embodiment a hydrotrope containing substance may comprise an imidazole or imidazolium group. In one embodiment a hydrotrope containing substance may comprise a tropane group.

In one embodiment a hydrotrope containing substance may comprise one or more pyridine or pyridinium group. In one embodiment a hydrotrope containing substance may comprise a pyridine alkaloid. In one embodiment a hydrotrope containing substance may comprise two or more pyridine or pyridinium groups, such as a bipyridine, as a non-limiting example. In one embodiment a hydrotrope containing substance may comprise three or more pyridine or pyridinium groups, four or more pyridine or pyridinium groups, five or more pyridine or pyridinium groups, or six or more pyridine or pyridinium groups.

In one embodiment a hydrotrope containing substance may comprise a xanthine or methylxanthine. In one embodiment a hydrotrope containing substance may comprise a purine or purinium, or a pyrimidine or pyrimidinium, wherein the purine or purinium, or the pyrimidine or pyrimidinium are not a nucleic acid base. In one embodiment a hydrotrope containing substance may comprise a purine alkaloid.

In one embodiment a hydrotrope containing substance may comprise one or more toluenesulfonyl group. In one embodiment a hydrotrope containing substance may comprise one or more toluenesulfonate group.

In one embodiment a hydrotrope containing substance may not comprise an aryl group. In one embodiment a hydrotrope containing substance may not comprise a phenyl group. In one embodiment a hydrotrope containing substance may not comprise a benzene ring. In one embodiment a hydrotrope containing substance may not comprise a phenol. In one embodiment a hydrotrope containing substance may not comprise a polyphenol. In one embodiment a hydrotrope containing substance may not comprise an aryloxy group. In one embodiment a hydrotrope containing substance may not comprise an alkoxy group.

In one embodiment a hydrotrope containing substance may not comprise a pyrrole group. In one embodiment a hydrotrope containing substance may not comprise a pyrroline group. In one embodiment a hydrotrope containing substance may not comprise pyrrolidine group. In one embodiment a hydrotrope containing substance may not comprise pyrrolidinium group. In one embodiment a hydrotrope containing substance may not comprise an indole group. In one embodiment a hydrotrope containing substance may not comprise a pyrrolizidine group. In one embodiment a hydrotrope containing substance may not comprise a piperidine group. In one embodiment a hydrotrope containing substance may not comprise a quinoline group. In one embodiment a hydrotrope containing substance may not comprise an isoquinoline group. In one embodiment a hydrotrope containing substance may not comprise a quinolizidine group. In one embodiment a hydrotrope containing substance may not comprise an indolizidine group. In one embodiment a hydrotrope containing substance may not comprise an oxazole group. In one embodiment a hydrotrope containing substance may not comprise an isoxazole group. In one embodiment a hydrotrope containing substance may not comprise a thiazole group. In one embodiment a hydrotrope containing substance may not comprise an acridine group. In one embodiment a hydrotrope containing substance may not comprise an imidazole or imidazolium group. In one embodiment a hydrotrope containing substance may not comprise a tropane group.

In one embodiment a hydrotrope containing substance may not comprise one or more pyridine or pyridinium group. In one embodiment a hydrotrope containing substance may not comprise a pyridine alkaloid. In one embodiment a hydrotrope containing substance may not comprise two or more pyridine or pyridinium groups, such as a bipyridine, as a non-limiting example. In one embodiment a hydrotrope containing substance may not comprise a xanthine or methylxanthine. In one embodiment a hydrotrope containing substance may not comprise a purine or pyrimidine. In one embodiment a hydrotrope containing substance may not comprise a purine alkaloid.

In one embodiment a hydrotrope containing substance may not comprise a toluenesulfonyl group. In one embodiment a hydrotrope containing substance may not comprise toluenesulfonate group.

In one embodiment a hydrotrope containing substance may be comprised of one or more gallate or gallic acid or one or more gallate or gallic acid derivative, such as ethyl gallate or epigallocatechin gallate as non-limiting examples. In one embodiment a hydrotrope containing substance may be comprised of one or more shikimate or shikimic acid or one or more shikimate or shikimic acid derivative.

In one embodiment a hydrotrope containing substance may be comprised of phenylalanine or tyrosine. In one embodiment a hydrotrope containing substance may be comprised of tryptophan. In one embodiment a hydrotrope containing substance may be comprised of one or more phenylalanine derivative or one or more tyrosine derivative, such as a phenylpropanoid, as a non-limiting example. In one embodiment a hydrotrope containing substance may be comprised of one or more tryptophan derivative, such as serotonin, melatonin, quinolinic acid, or nicotinic acid as non-limiting examples.

In one embodiment a hydrotrope containing substance may comprise one or more coumaric acid or coumaric acid derivative.

In one embodiment a hydrotrope containing substance may be comprised of one or more phenylpropanoid or one or more phenylpropanoid derivative, such as one or more cinnamic acids, hydroxycinnamic acids, cinnamic aldehydes, monolignols, coumarins, flavonoids, or stilbenoids, as non-limiting examples.

In one embodiment a hydrotrope containing substance may not comprise gallate or gallic acid or gallate or gallic acid derivative. In one embodiment a hydrotrope containing substance may not comprise shikimate or shikimic acid or a shikimate or shikimic acid derivative.

In one embodiment a hydrotrope containing substance may not comprise phenylalanine or tyrosine. In one embodiment a hydrotrope containing substance may not comprise tryptophan. In one embodiment a hydrotrope containing substance may not comprise a phenylalanine derivative or tyrosine derivative. In one embodiment a hydrotrope containing substance may not comprise a tryptophan derivative. In one embodiment a hydrotrope containing substance may not comprise a peptide or polypeptide.

In one embodiment a hydrotrope containing substance may not comprise a coumaric acid or a coumaric acid derivative.

In one embodiment a hydrotrope containing substance may not comprise a phenylpropanoid or phenylpropanoid derivative.

In one embodiment a hydrotrope containing substance may comprise one or more of the following formulas:

G-(J)ₚ or G-(W)ₜ or (J)ₚ-G-(W)ₜ or G-(JW)ᵢ or (J)ₚ-G-(JW)ᵢ or (W)ₜ-G-(JW)ᵢ

such as the following non-limiting examples:

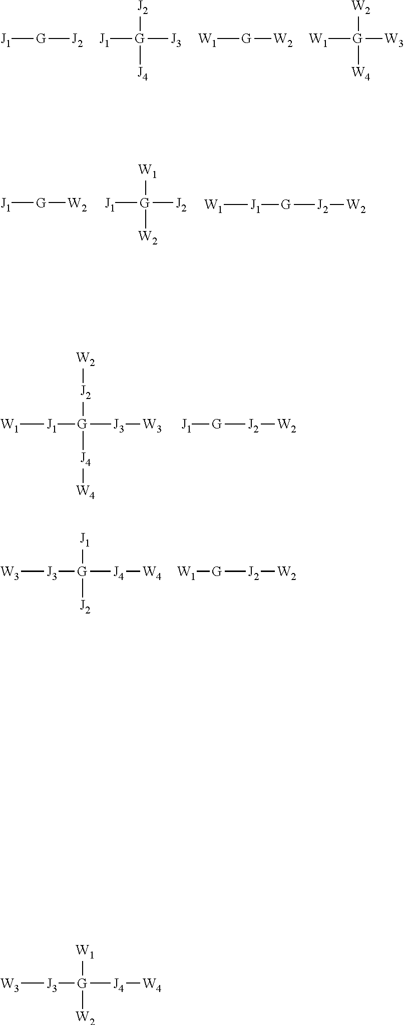

Wherein, t, p, and i may be independently selected integers between about 1-20, or between about 1-10, or between about 1-6, or between about 1-5 or between about 1-4, or between about 1-3, or between about 1-2, such that t, p, or i may be equal to 1, 2, 3, 4, 5, 6, 7, 8, 9, 10, 11, 12, 13, 14, 15, 16, 17, 18, 19, or 20, as non-limiting examples. G is a G group comprised of at least one or more cyclic ring structure, such as a five membered ring structure or a six membered ring structure as non-limiting examples, and a G group may be covalently bonded to one or more J group, or one or more W group, or one or more JW group. J is a J group comprised of at least one carbon atom. W is a W group comprised of at least one oxygen atom or at least one nitrogen atom. Furthermore, a J group may be covalently bonded to a W group, wherein a JW group comprises a J group covalently bonded to a W group.

Furthermore, a hydrotrope containing substance may comprise one or more of the following formulas: (J)ₚ/(W)ₜ-G-(JW)ᵢ, such that a G group may be covalently bonded to one or more JW group, and one or more J group, and one or more W group, wherein (J)/(W) may be one or more J group or one or more W group bonded to a G group, such as the following non-limiting examples:

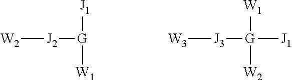

In one embodiment a G group may be covalently bonded to one or more J groups, or one or more W groups, or one or more JW groups, wherein the number of J groups, W groups, or JW groups may be the same or different.

In one embodiment t or p may be equal to i. In embodiment t or p may not be equal to i. In one embodiment t or p may be equal to each other. In one embodiment t or p may not be equal to each other.

In one embodiment a G group may be comprised of one or more of the following: phenyl, phenol, aryl, pyridine, pyridinium, pyrrolidine, pyrrolidinium, pyrrole, imidazole, imidazolium, pyrazole, purine, purinium, pyrimidine, pyrimidinium, xanthine, piperidine, piperidinium, indole, indolium, methylxanthine, polyphenol, phenylalanine derivate, tyrosine derivative, tryptophan derivative, phenylpropanoid or phenylpropanoid derivative.

In one embodiment one or more W groups (such as W₁, W₂, W₃, or W₄, as non-limiting examples) may be independently selected W groups, wherein one or more W groups (such as W₁, W₂, W₃, or W₄, as non-limiting examples) may be the same or different.

In one embodiment, one or more W groups (such as W₁, W₂, W₃, or W₄, as non-limiting examples) may be interchangeable with one or more other W groups. As a non-limiting example, W₁ may be interchangeable with one or more other W group such as W₂, W₃, or W₄, wherein W₁ may be replaced or exchange positions with one or more other W group such as W₂, W₃, or W₄.

In one embodiment one or more W group may be comprised of one or more of the following: a carboxylate or carboxylic acid group, or an amide group, or a hydroxy group, or an ammonium group, or a quaternary ammonium group, or a tertiary amine group, or a secondary amine group, or primary amine group, or an organosulfate group, or a sulfonate group, or a phosphonate group, or an organophosphate group

In one embodiment a hydrotrope containing substance may not comprise one or more J group. In one embodiment a hydrotrope containing substance may not comprise one or more W group. In one embodiment a hydrotrope containing substance may comprise one or more J group without a W group. In one embodiment a hydrotrope containing substance may comprise one or more W group without a J group. In one embodiment a hydrotrope containing substance may comprise an unequal number of W groups or J groups. In one embodiment a hydrotrope containing substance may comprise one or more J groups, wherein the J groups may be the same or different. In one embodiment a hydrotrope containing substance may comprise one or more W groups, wherein the W groups may be the same or different.

In one embodiment one or more J groups may be covalently bonded to one or more G group. In one embodiment one or more W groups may be covalently bonded to one or more G group. In one embodiment one or more W groups may be covalently bonded to one or more J groups.

In one embodiment one or more J group may not be covalently bonded to one or more G group. In one embodiment one or more W group may not be covalently bonded to one or more G group. In one embodiment one or more W group may not be covalently bonded to one or more J group.

In one embodiment one or more J groups (such as J₁, J₂, J₃, or J₄, as non-limiting examples) may be independently selected J groups, wherein one or more J groups (such as J₁, J₂, J₃, or J₄, as non-limiting examples) may be the same or different.

In one embodiment, one or more J groups (such as J₁, J₂, J₃, or J₄, as non-limiting examples) may be interchangeable with one or more other J groups. As a non-limiting example, J₁ may be interchangeable with one or more other J group such as J₂, J₃, or J₄, wherein J₁ may be replaced or exchange positions with one or more other J group such as J₂, J₃, or J₄.

In one embodiment a J group may be comprised of at least one carbon atom. Wherein, J may be comprised of a C1-35 alkyl, alkenyl, alkynyl, aryl, or aralkyl group, each of which may contain up to 20 heteroatoms. In another embodiment a J group may be comprised of C1-30 alkyl, alkenyl, alkynyl, aryl, or aralkyl group, each of which may contain up to 16 heteroatoms. In another embodiment a J group may be comprised of C1-24 alkyl, alkenyl, alkynyl, aryl, or aralkyl group, each of which may contain up to 12 heteroatoms. In another embodiment a J group may be comprised of C1-20 alkyl, alkenyl, alkynyl, aryl, or aralkyl group, each of which may contain up to 10 heteroatoms. In another embodiment a J group may be comprised of C1-16 alkyl, alkenyl, alkynyl, aryl, or aralkyl group, each of which may contain up to 8 heteroatoms. In another embodiment a J group may be comprised of C1-12 alkyl, alkenyl, alkynyl, aryl, or aralkyl group, each of which may contain up to 6 heteroatoms. In another embodiment a J group may be comprised of C1-12 alkyl, alkenyl, alkynyl, aryl, or aralkyl group, each of which may contain up to 4 heteroatoms. In another embodiment a J group may be comprised of C1-8 alkyl, alkenyl, alkynyl, aryl, or aralkyl group, each of which may contain up to 4 heteroatoms. In another embodiment a J group may be comprised of C1-8 alkyl, alkenyl, alkynyl, aryl, or aralkyl group, each of which may contain up to 3 heteroatoms. In another embodiment a J group may be comprised of C1-6 alkyl, alkenyl, alkynyl, aryl, or aralkyl group, each of which may contain up to 3 heteroatoms. In another embodiment a J group may be comprised of C1-6 alkyl, alkenyl, alkynyl, aryl, or aralkyl group, each of which may contain up to 2 heteroatoms. In another embodiment a J group may be comprised of C1-4 alkyl, alkenyl, or alkynyl group, each of which may contain up to 2 heteroatoms.

In one embodiment one or more J groups may be aliphatic.

Embodiments of the present invention comprising a hydrotrope containing substance may comprise one or more J groups wherein a J group may comprise one or more nitrogen atom or one or more oxygen atom or one or more sulfur atom or one or more phosphorus atom. Embodiments of the present invention comprising a hydrotrope containing substance may comprise one or more J groups wherein a J group may comprise, one or more of the following groups, including but not limited to: hydroxy groups, alcohols, alkyl groups, alkenyl groups, alkynyl groups, aryl groups, aralkyl groups, ether groups, ethers, carboxamide groups, amides, carboalkoxy groups, esters, carbonate esters, or carboxylate esters.

In one embodiment a J group may not comprise a nitrogen atom. In one embodiment a J group may not comprise an oxygen atom. In one embodiment a J group may not be comprise a sulfur atom. In one embodiment a J group may not be comprise a phosphorus atom. In one embodiment a J group may not comprise one or more of the following groups: hydroxy groups, alcohols, alkyl groups, alkenyl groups, alkynyl groups, aryl groups, aralkyl groups, ether groups, ethers, carboxamide groups, amides, carboalkoxy groups, esters, carbonate esters, or carboxylate esters.

In one embodiment a J group may be covalently bonded to another J group. In one embodiment two or more J groups may be covalently bonded to one or more J groups.

In one embodiment one or more J group may be covalently bonded to one or more adjacent J group, such as in a cyclic or heterocyclic ring structure, as a non-limiting example. In one embodiment two or more J groups may be covalently bonded together, such as in a cyclic or heterocyclic ring structure, as a non-limiting example. In one embodiment one or more J groups may form a heterocyclic ring structure with one or more covalently bonded nitrogen atom, or one or more covalently bonded sulfur atom, or one or more covalently bonded phosphorus atom.

In one embodiment one or more J groups may form a ring structure. In another embodiment one or more J groups may form at least part of a ring structure. In another embodiment one or more J groups may be at least part of a ring structure. In another embodiment one or more J groups may comprise at least part of a ring structure.

In another embodiment one or more J groups may form a heterocyclic ring structure. In one embodiment one or more J groups may form at least part of a heterocyclic ring structure. In one embodiment one or more J groups may be at least part of a heterocyclic ring structure. In another embodiment one or more J groups may comprise at least part of a heterocyclic ring structure.

In one embodiment one or more J groups may not form a ring structure. In another embodiment one or more J groups may not be covalently bonded to another J group. In another embodiment one or more J groups may not be part of a ring structure. In another embodiment one or more J groups may not comprise a ring structure.

A non-limiting example of a hydrotrope containing substance comprised of the formula G-(J)ₚ is 1-butyl-1-methylpyrrolidinium, wherein G is a five membered ring structure comprised of a pyrrolidinium and p is equal to 2 where J₁ is a methyl group, and J₂ is a butyl group.

A non-limiting example of a hydrotrope containing substance comprised of the formula G-(W)ₜ is benzoate, wherein G is six membered ring structure comprised of a phenyl group and t is equal to 1 where W₁ is a carboxylate group.

A non-limiting example of a hydrotrope containing substance comprised of the formula G-(JW)ᵢ is N,N-dimethylphenethylamine wherein G is a six membered ring structure comprised of a phenyl group and i is equal to 1 and JW is comprised of a J group bonded to a W group where J is an ethyl group and W is a tertiary amine.

A non-limiting example of a hydrotrope containing substance comprised of the formula (J)ₚ-G-(W)ₜ is 1-methylnicotinamide wherein G is a six membered ring structure comprised of a pyridinium group and p is equal to 1 and J₁ is a methyl group and t is equal to 1 and Wᵢ is an amide group.

A non-limiting example of a hydrotrope containing substance comprised of the formula (W)ₜ-G-(JW)ᵢ is caffeic acid wherein G is an aryl group and t is equal to 2 and W₁ and W₂ are hydroxy groups and JW is comprised of a J group bonded to a W group where J is an ethylene group and W is a carboxylic acid group.

A non-limiting example of an anionic hydrotrope containing substance, comprised of one or more phenyl group and one or more carboxylate group, that may be used is benzoate (also known as benzoic acid), such as sodium benzoate as a non-limiting example. One embodiment of the present invention may be a combination of substances comprising benzoate and another embodiment may also comprise at least one RNA substance. Other embodiments may be combinations of substances comprising benzoate wherein the concentration of benzoate may be one or more concentration in the stabilizing substance weight percent concentration list or in the stabilizing substance molar concentration range list or in the stabilizing substance molar concentration list.

A non-limiting example of a cationic hydrotrope containing substance, comprised of one or more heterocyclic five membered ring or one or more pyrrolidinium group, that may be used is 1-butyl-1-methylpyrrolidinium, such as 1-butyl-1-methylpyrrolidinium bromide as a non-limiting example, wherein 1-butyl-1-methylpyrrolidinium may be substituted for or used in combination with one or more hydrotrope containing substance as described herein. One embodiment of the present invention may include combinations of substances comprising 1-butyl-1-methylpyrrolidinium and another embodiment may also comprise at least one RNA substance. Other embodiments may include combinations of substances comprising 1-butyl-1-methylpyrrolidinium wherein the concentration of 1-butyl-1-methylpyrrolidinium may be one or more concentration in the stabilizing substance weight percent concentration list or in the stabilizing substance molar concentration range list or in the stabilizing substance molar concentration list.

A non-limiting example of a cationic hydrotrope containing substance, comprised of one or more phenyl group, that may be used is benzyltriethylammonium, such as benzyltriethylammonium chloride as a non-limiting example, wherein benzyltriethylammonium may be substituted for or used in combination with one or more hydrotrope containing substance as described herein. One embodiment of the present invention may include combinations of substances comprising benzyltriethylammonium and another embodiment may also comprise at least one RNA substance. Other embodiments may include combinations of substances comprising benzyltriethylammonium wherein the concentration of benzyltriethylammonium may be one or more concentration in the stabilizing substance weight percent concentration list or in the stabilizing substance molar concentration range list or in the stabilizing substance molar concentration list.

A non-limiting example of a hydrotrope containing substance, comprised of one or more phenyl group, that may be used is N,N-dimethylphenethylamine, wherein N,N-dimethylphenethylamine may be substituted for or used in combination with one or more hydrotrope containing substance as described herein. One embodiment of the present invention may include combinations of substances comprising N,N-dimethylphenethylamine and another embodiment may also comprise at least one RNA substance. Other embodiments may include combinations of substances comprising N,N-dimethylphenethylamine wherein the concentration of N,N-dimethylphenethylamine may be one or more concentration in the stabilizing substance weight percent concentration list or in the stabilizing substance molar concentration range list or in the stabilizing substance molar concentration list.

A non-limiting example of a cationic hydrotrope containing substance, comprised of one or more phenyl group and one or more imidazolium group, that may be used is 1-benzyl-3-methylimidazolium, such as 1-benzyl-3-methylimidazolium chloride as a non-limiting example, wherein 1-benzyl-3-methylimidazolium may be substituted for or used in combination with one or more hydrotrope containing substance as described herein. One embodiment of the present invention may include combinations of substances comprising 1-benzyl-3-methylimidazolium and another embodiment may also comprise at least one RNA substance. Other embodiments may include combinations of substances comprising 1-benzyl-3-methylimidazolium wherein the concentration of 1-benzyl-3-methylimidazolium may be one or more concentration in the stabilizing substance weight percent concentration list or in the stabilizing substance molar concentration range list or in the stabilizing substance molar concentration list.

A non-limiting example of a cationic hydrotrope containing substance, comprised of one or more pyridinium group, that may be used is 1-butylpyridinium, such as 1-butylpyridinium bromide as a non-limiting example, wherein 1-butylpyridinium may be substituted for or used in combination with one or more hydrotrope containing substance as described herein. One embodiment of the present invention may include combinations of substances comprising 1-butylpyridinium and another embodiment may also comprise at least one RNA substance. Other embodiments may include combinations of substances comprising 1-butylpyridinium wherein the concentration of 1-butylpyridinium may be one or more concentration in the stabilizing substance weight percent concentration list or in the stabilizing substance molar concentration range list or in the stabilizing substance molar concentration list.

A non-limiting example of a hydrotrope containing substance, comprised of one or more pyridine group and one or more carboxylate group, that may be used is nicotinic acid (also known as niacin), wherein nicotinic acid may be substituted for or used in combination with one or more hydrotrope containing substance as described herein. One embodiment of the present invention may include combinations of substances comprising nicotinic acid and another embodiment may also comprise at least one RNA substance. Other embodiments may include combinations of substances comprising nicotinic acid wherein the concentration of nicotinic acid may be one or more concentration in the stabilizing substance weight percent concentration list or in the stabilizing substance molar concentration range list or in the stabilizing substance molar concentration list.

A non-limiting example of a hydrotrope containing substance, comprised of one or more pyridine group and one or more carboxylate group, that may be used is quinolinic acid (also known as pyridine-2,3-dicarboxylic acid), wherein quinolinic acid may be substituted for or used in combination with one or more hydrotrope containing substance as described herein. One embodiment of the present invention may include combinations of substances comprising quinolinic acid and another embodiment may also comprise at least one RNA substance. Other embodiments may include combinations of substances comprising quinolinic acid wherein the concentration of quinolinic acid may be one or more concentration in the stabilizing substance weight percent concentration list or in the stabilizing substance molar concentration range list or in the stabilizing substance molar concentration list.

A non-limiting example of a cationic hydrotrope containing substance, comprised of one or more pyridinium group and one or more amide group, that may be used is 1-methylnicotinamide (also known as 3-carbamyl-1-methylpyridinium or trigonellamide), such as 1-methylnicotinamide chloride as a non-limiting example, wherein 1-methylnicotinamide may be substituted for or used in combination with one or more hydrotrope containing substance as described herein. One embodiment of the present invention may include combinations of substances comprising 1-methylnicotinamide and another embodiment may also comprise at least one RNA substance. Other embodiments may include combinations of substances comprising 1-methylnicotinamide wherein the concentration of 1-methylnicotinamide may be one or more concentration in the stabilizing substance weight percent concentration list or in the stabilizing substance molar concentration range list or in the stabilizing substance molar concentration list.

A non-limiting example of a hydrotrope containing substance, comprised of one or more pyridine or pyridinium group and one or more amide group, that may be used is nicotinamide N-oxide, wherein nicotinamide N-oxide may be substituted for or used in combination with one or more hydrotrope containing substance as described herein. One embodiment of the present invention may include combinations of substances comprising nicotinamide N-oxide and another embodiment may also comprise at least one RNA substance. Other embodiments may include combinations of substances comprising nicotinamide N-oxide wherein the concentration of nicotinamide N-oxide may be one or more concentration in the stabilizing substance weight percent concentration list or in the stabilizing substance molar concentration range list or in the stabilizing substance molar concentration list.

For conciseness, the following list of hydrotrope containing substances is herein referred to as the hydrotrope containing substance list wherein one or more of the following substances may be substituted for or used in combination with one or more hydrotrope containing substance as described herein.

In other embodiments, other hydrotrope containing substances that may be substituted for or used in combination with one or more hydrotrope containing substance as described herein may comprise one or more of the following, including but not limited to: benzoate, 1-butyl-1-methylpyrrolidinium, benzyltriethylammonium, N,N-dimethylphenethylamine, 1-benzyl-3-methylimidazolium, 1-butylpyridinium, nicotinic acid, quinolinic acid, 1-methylnicotinamide, nicotinamide N-oxide, nicotinic acid N-oxide, dipicolinic acid, isonicotinamide, kairine, berberonic acid, caffeine, cafaminol, picolinic acid, theacrine, thiamine, theobromine, methylliberine, gallic acid, hexahydroxydiphenic acid, ellagic acid, urolithins, such as urolithin A as a non-limiting example, ellagitannins, such as casuarictin as a non-limiting example, luteic acid, depsides, such as gyrophoric acid, as a non-limiting example, p-toluenesulfonic acid, nicotine, doxylamine, N,N-diethylnicotinamide, N,N-dimethylnicotinamide, 2-quinolone, 2-quinoxalinol, trimesic acid, phenylpropanoids, phenylalanine, tyrosine, gallate, gallic acids, shikimate, shikimic acids, coumaric acids, cinnamic acids, hydroxycinnamic acids, cinnamic aldehydes, monolignols, coumarins, flavonoids, stilbenoids, or cotinine, or salts, or derivatives, or combinations thereof.

Embodiments of the present invention may include combinations comprising one or more of the substances from the hydrotrope containing substance list substituted for or used in combination with one or more hydrotrope containing substance as described herein and other embodiments may also comprise at least one RNA substance.

Other embodiments may include combinations of substances comprising one or more hydrotrope containing substance selected from the hydrotrope containing substance list wherein the concentration of one or more substances may be one or more concentration in the stabilizing substance weight percent concentration list or in the stabilizing substance molar concentration range list or in the stabilizing substance molar concentration list.

In one embodiment, a hydrotrope containing substance may have a molecular weight between about 50-5,000 daltons. In one embodiment, a hydrotrope containing substance may have a molecular weight between about 50-2,000 daltons. In one embodiment, a hydrotrope containing substance may have a molecular weight between about 50-1,000 daltons. In one embodiment, a hydrotrope containing substance may have a molecular weight between about 50-900 daltons. In one embodiment, a hydrotrope containing substance may have a molecular weight between about 50-800 daltons. In one embodiment, a hydrotrope containing substance may have a molecular weight between about 50-700 daltons. In one embodiment, a hydrotrope containing substance may have a molecular weight between about 50-600 daltons. In one embodiment, a hydrotrope containing substance may have a molecular weight between about 50-500 daltons. In one embodiment, a hydrotrope containing substance may have a molecular weight between about 50-400 daltons. In one embodiment, a hydrotrope containing substance may have a molecular weight between about 50-300 daltons. In one embodiment, a hydrotrope containing substance may have a molecular weight between about 50-250 daltons.

In one embodiment, a hydrotrope containing substance may have a molecular weight between about 100-5,000 daltons. In one embodiment, a hydrotrope containing substance may have a molecular weight between about 100-2,000 daltons. In one embodiment, a hydrotrope containing substance may have a molecular weight between about 100-1,000 daltons. In one embodiment, a hydrotrope containing substance may have a molecular weight between about 100-900 daltons. In one embodiment, a hydrotrope containing substance may have a molecular weight between about 100-800 daltons. In one embodiment, a hydrotrope containing substance may have a molecular weight between about 100-700 daltons. In one embodiment, a hydrotrope containing substance may have a molecular weight between about 100-600 daltons. In one embodiment, a hydrotrope containing substance may have a molecular weight between about 100-500 daltons. In one embodiment, a hydrotrope containing substance may have a molecular weight between about 100-400 daltons. In one embodiment, a hydrotrope containing substance may have a molecular weight between about 100-300 daltons. In one embodiment, a hydrotrope containing substance may have a molecular weight between about 100-250 daltons.

One embodiment of the present invention is the method whereby one or more hydrotrope containing substance may be combined, such as by mixing, with at least one or more RNA substance to produce a mixture comprising at least one or more hydrotrope containing substance and at least one or more RNA substance. As a non-limiting example one or more RNA substance may be mixed with one or more hydrotrope containing substance.

Another embodiment of the present invention is the method whereby one or more hydrotrope containing substance may be combined, such as by mixing, with at least one or more RNA substance to produce a mixture comprising at least one or more RNA substance and at least one or more hydrotrope containing substance at one or more of the RNA stabilizing substance concentrations within the stabilizing substance molar concentration range list or in the stabilizing substance molar concentration list or the stabilizing substance weight percent concentration list as described herein. These same methods may be used to combine one or more other substances, including, but not limited to one or more cellular uptake agents or one or more additional RNA stabilizing substances, with one or more RNA substances and one or more hydrotrope containing substances to produce a mixture comprising one or more RNA substance, one or more hydrotrope containing substance, and one or more cellular uptake agent or one or more additional RNA stabilizing substance.

One embodiment of the present invention is the method whereby one or more hydrotrope containing substance may be combined, such as by mixing, with at least one or more RNA substance to produce a composition comprising at least one or more RNA substance and at least one or more hydrotrope containing substance. Another embodiment of the present invention is the method whereby one or more hydrotrope containing substance may be combined, such as by mixing, with one or more RNA substance to produce a composition comprising at least one or more RNA substance and at least one or more hydrotrope containing substance at one or more of the RNA stabilizing substance concentrations within the stabilizing substance molar concentration range list or in the stabilizing substance molar concentration list or stabilizing substance weight percent concentration list as described herein. These same methods may be used to combine one or more other substances, including, but not limited to one or more cellular uptake agents or one or more additional RNA stabilizing substance, with one or more RNA substance and one or more hydrotrope containing substance to produce a composition comprising one or more RNA substance, one or more hydrotrope containing substance, and one or more cellular uptake agent or one or more additional RNA stabilizing substance.

In one embodiment a hydrotrope containing substance may not be aliphatic. In one embodiment a hydrotrope containing substance may comprise a ring structure. In another embodiment a hydrotrope containing substance may comprise a heterocyclic ring structure. In one embodiment a hydrotrope containing substance may comprise an aromatic ring structure. In another embodiment a hydrotrope containing substance may comprise an aromatic heterocyclic ring structure.

In one embodiment a hydrotrope containing substance may comprise a cationic moiety at about physiologic pH. In one embodiment a hydrotrope containing substance may comprise an anionic moiety at about physiologic pH. In one embodiment a hydrotrope containing substance may comprise a zwitterion at about physiologic pH.

In one embodiment a hydrotrope containing substance may comprise one or more PIF as described herein.

In one embodiment, one or more hydrotrope containing substance may be at least part of a polymer comprised of at least one or more hydrotrope containing substances.

In one embodiment one or more composition comprising one or more RNA substance and one or more hydrotrope containing substance may comprise one or more water concentrations in the water concentration list or in the water concentration range list. In one embodiment one or more composition comprising one or more RNA substance, one or more hydrotrope containing substance, and one or more additional substance, such as one or more cellular uptake agent or one or more additional RNA stabilizing substance as non-limiting examples, may comprise one or more water concentrations in the water concentration list or in the water concentration range list. In one embodiment one or more composition comprising one or more RNA substance and one or more hydrotrope containing substance may have a dielectric constant of one or more dielectric constant in the dielectric constant list or in the dielectric constant range list. In one embodiment one or more composition comprising one or more RNA substance, one or more hydrotrope containing substance, and one or more additional substance, such as one or more cellular uptake agent or one or more additional RNA stabilizing substance as non-limiting examples, may have a dielectric constant of one or more dielectric constant in the dielectric constant list or in the dielectric constant range list.

Embodiments of the present invention may include compositions comprising one or more hydrotrope containing substance used in a composition comprising one or more RNA substance, one or more hydrotrope containing substance, and one or more of the following substances: water, cellular uptake agents, aprotic substances, low dielectric substances, cyclic phosphate or metaphosphate containing substances, non-carbohydrate organic osmolyte substances, tertiary sulfonium containing substances, quaternary ammonium containing substances, quaternary phosphonium containing substances, surfactant containing substances, betaine containing substances, stabilizing monomers, stabilizing polymers, or supplemental RNA stabilizing substances. Other embodiments may include one or more composition comprising one or more hydrotrope containing substance and one or more RNA substance and may also comprise one or more cellular uptake agent or one or more additional RNA stabilizing substances. Other embodiments may include compositions comprising one or more hydrotrope containing substance, one or more RNA substance, and one or more additional substances, such as: one or more cellular uptake agent, or one or more additional RNA stabilizing substance, or one or more buffering agent, or one or more chelating agent, or one or more inorganic salt, or water, as non-limiting examples.

Embodiments of the present invention may include one or more compositions comprising one or more RNA substance and one or more hydrotrope containing substance, wherein a composition is not lyophilized.

In one embodiment one or more composition comprising one or more RNA substance and one or more hydrotrope containing substance may not be lyophilized. In one embodiment one or more composition comprising one or more RNA substance, one or more hydrotrope containing substance, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more hydrotrope containing substance, wherein a composition may be lyophilized.

In one embodiment one or more composition comprising one or more RNA substance and one or more hydrotrope containing substance may be lyophilized. In one embodiment one or more composition comprising one or more RNA substance, one or more hydrotrope containing substance, and one or more additional substance, such as one or more cellular uptake agent or one or more additional RNA stabilizing substance as non-limiting examples, may be lyophilized.

Embodiments of the present invention may include one or more compositions comprising one or more hydrotrope containing substance and one or more RNA substance described herein, wherein one or more composition described herein may have a melting of one or more melting point in the stabilizing composition melting point list.

In one embodiment one or more composition comprising one or more RNA substance and one or more hydrotrope containing substance may have a melting point of one or more melting point in the stabilizing composition melting point list. In one embodiment one or more composition comprising one or more RNA substance, one or more hydrotrope containing substance, and one or more additional substance, such as one or more cellular uptake agent or one or more additional RNA stabilizing substance as a non-limiting example, may have a melting point of one or more melting point in the stabilizing composition melting point list.

### Surfactant Containing Substances:

The inventors have discovered that surfactant containing substances may stabilize RNA substances.

The inventors have discovered that RNA stabilizing substances may comprise surfactant containing substances. The inventors have also discovered that the stability of RNA substances may be enhanced in an RNA storage environment comprising a surfactant containing substance. The inventors have also discovered that the stability of RNA substances may be enhanced in an RNA storage environment comprising a surfactant containing substance and one or more additional RNA stabilizing substance, such as an aprotic substance.

The inventors have also discovered that the stability of RNA substances may be enhanced in compositions comprising an RNA substance and a surfactant containing substance. The inventors have also discovered that the stability of RNA substances may be enhanced in compositions comprising an RNA substance, a surfactant containing substance, and one or more additional RNA stabilizing substance, such as an aprotic substance.

In one embodiment of the present invention, one or more RNA stabilizing substance may comprise a surfactant containing substance.

One embodiment of the present invention may include a composition comprised of one or more RNA substance and one or more surfactant containing substance. Another embodiment of the present invention may include a composition comprised of one or more RNA substance, one or more surfactant containing substance, and one or more additional RNA stabilizing substance, such as an aprotic substance.

One embodiment of the present invention may include a combination or mixture comprising one or more RNA substance and one or more surfactant containing substance. Another embodiment of the present invention may include a combination or mixture comprising one or more RNA substance, one or more surfactant containing substance, and one or more additional RNA stabilizing substance, such as an aprotic substance.

Embodiments of the present invention that comprise one or more RNA substance and one or more surfactant containing substance may include combining, such as by mixing, one or more RNA substance with one or more substance that comprises at least one or more surfactant containing substance.

Embodiments of the present invention that comprise one or more RNA substance, one or more surfactant containing substance, and one or more additional RNA stabilizing substance, such as an aprotic substance, may include combining, such as by mixing, one or more RNA substance with one or more substance that comprises at least one or more surfactant containing substance and one or more substance that comprises at least one or more RNA stabilizing substance, such as an aprotic substance.

An embodiment of the present invention may include compositions of materials that comprise one or more RNA substance and at least one or more surfactant containing substance. The storage environment that improves the stability of RNA substances may comprise at least one or more vapor, liquid, powder, or solid surfactant containing substance.

An embodiment of the present invention may include compositions of materials that comprise one or more RNA substance, one or more surfactant containing substance, and one or more additional RNA stabilizing substance, such as an aprotic substance. The storage environment that improves the stability of RNA substances may comprise at least one or more vapor, liquid, powder, or solid surfactant containing substance and may comprise at least one or more vapor, liquid, powder, or solid RNA stabilizing substance, such as an aprotic substance.

An embodiment of the present invention may include combinations of materials that comprise one or more RNA substance and at least one or more surfactant containing substance. The storage environment that improves the stability of RNA substances may comprise at least one or more vapor, liquid, powder, or solid surfactant containing substance.

An embodiment of the present invention may include combinations of materials that comprise one or more RNA substance, one or more surfactant containing substance, and one or more additional RNA stabilizing substance, such as an aprotic substance. The storage environment that improves the stability of RNA substances may comprise at least one or more vapor, liquid, powder, or solid surfactant containing substance and may comprise at least one or more vapor, liquid, powder, or solid RNA stabilizing substance, such as an aprotic substance.

In one embodiment of the present invention a composition comprised of one or more RNA substance and one or more surfactant containing substance, produces a mixture with at least one or more RNA substance and at least one or more surfactant containing substance. In one embodiment of the present invention a composition comprised of one or more RNA substance, one or more surfactant containing substance, and one or more additional RNA stabilizing substance, such as an aprotic substance, produces a mixture with at least one or more RNA substance, at least one or more surfactant containing substance, and at least one or more additional RNA stabilizing substance, such as an aprotic substance.

In one embodiment of the present invention a combination comprising one or more RNA substance and one or more surfactant containing substance, produces a mixture with at least one or more RNA substance and at least one or more surfactant containing substance. In one embodiment of the present invention a combination comprising one or more RNA substance, one or more surfactant containing substance, and one or more additional RNA stabilizing substance, such as an aprotic substance, produces a mixture with at least one or more RNA substance, at least one or more surfactant containing substance, and at least one or more additional RNA stabilizing substance, such as an aprotic substance.

In one embodiment of the present invention a composition with improved RNA stability may comprise one or more RNA substance and one or more surfactant containing substance. In one embodiment of the present invention a composition with improved RNA stability may comprise one or more RNA substance, one or more surfactant containing substance, and one or more additional RNA stabilizing substance, such as an aprotic substance.

In one embodiment of the present invention a composition with improved RNA stability may comprise a mixture of one or more RNA substance and one or more surfactant containing substance. In one embodiment of the present invention a composition with improved RNA stability may comprise a mixture of one or more RNA substance, one or more surfactant containing substance, and one or more additional RNA stabilizing substance, such as an aprotic substance.

In one embodiment of the present invention, each component included in a composition comprising one or more RNA substance and one or more surfactant containing substance, may be stored separately, such as in a kit, or such as individual substances or as mixtures of one or more substance, and then combined later to produce a composition comprising one or more RNA substance and one or more surfactant containing substance.

In one embodiment of the present invention, each component included in a composition comprising one or more RNA substance, one or more surfactant containing substance, and one or more additional RNA stabilizing substance, such as an aprotic substance, may be stored separately, such as in a kit, or such as individually or as mixtures of one or more substance, and then combined later to produce a composition comprising one or more RNA substance, one or more surfactant containing substance, and one or more additional RNA stabilizing substance, such as an aprotic substance.

In one embodiment of the present invention a composition comprising one or more RNA substance and one or more surfactant containing substance may be at least partially biocompatible.

In one embodiment of the present invention a composition comprising one or more RNA substance, one or more surfactant containing substance, and one or more additional RNA stabilizing substance, such as an aprotic substance, may be at least partially biocompatible. In one embodiment of the present invention a combination comprised of one or more RNA substance and one or more surfactant containing substance may be at least partially biocompatible.

In one embodiment of the present invention a combination comprised of one or more RNA substance, one or more surfactant containing substance, and one or more additional RNA stabilizing substance, such as an aprotic substance, may be at least partially biocompatible.

Embodiments of the present invention comprising at least one or more surfactant containing substances may include one or more forms of the substance. These forms may include, but are not limited to, one more counterions, ionic forms, conjugate bases, conjugate acids, protonated or deprotonated forms, hydrated or dehydrated forms, isomers, structural isomers, stereo isomers, chiral forms, salts, or combinations thereof.

In one embodiment one or more surfactant containing substance may comprise one or more anionic moiety. In one embodiment one or more surfactant containing substance may comprise one or more cationic moiety. In one embodiment one or more surfactant containing substance may comprise one or more anionic moiety and one or more cationic moiety. In one embodiment one or more surfactant containing substance may comprise one or more zwitterionic moiety. In one embodiment a surfactant containing substance may be zwitterionic.

In one embodiment a surfactant containing substance may be comprised of one or more carboxylic acid or carboxylate group, or one or more organophosphate group, or one or more organosulfate group, or one or more sulfonic acid or sulfonate group, or one or more phosphonic acid or phosphonate group.

In one embodiment a surfactant containing substance may comprise a cyclic ring structure. In one embodiment a surfactant containing substance may comprise a heterocyclic ring structure. In one embodiment a surfactant containing substance may comprise an aromatic cyclic ring structure. In one embodiment a surfactant containing substance may comprise an aromatic heterocyclic ring structure. In one embodiment a surfactant containing substance may comprise a nitrogen heterocycle. In one embodiment a surfactant containing substance may comprise an aromatic nitrogen heterocycle.

In one embodiment a surfactant containing substance may comprise a five membered ring structure. In one embodiment a surfactant containing substance may comprise an aromatic five membered ring structure. In one embodiment a surfactant containing substance may comprise a heterocyclic five membered ring structure. In one embodiment a surfactant containing substance may comprise an aromatic heterocyclic five membered ring structure. In one embodiment a surfactant containing substance may comprise a five membered nitrogen heterocycle. In one embodiment a surfactant containing substance may comprise an aromatic five membered nitrogen heterocycle.

In one embodiment a surfactant containing substance may comprise a six membered ring structure. In one embodiment a surfactant containing substance may comprise an aromatic six membered ring structure. In one embodiment a surfactant containing substance may comprise a heterocyclic six membered ring structure. In one embodiment a surfactant containing substance may comprise an aromatic heterocyclic six membered ring structure. In one embodiment a surfactant containing substance may comprise a six membered nitrogen heterocycle. In one embodiment a surfactant containing substance may comprise an aromatic six membered nitrogen heterocycle.

In one embodiment a surfactant containing substance may comprise an aryl group. In one embodiment a surfactant containing substance may comprise a phenyl group. In one embodiment a surfactant containing substance may comprise a benzene ring. In one embodiment a surfactant containing substance may comprise a phenol, wherein a phenyl group may be bonded to one or more hydroxyl groups, two more hydroxyl groups, three or more hydroxyl groups, four or more hydroxyl groups or five or more hydroxyl groups. In one embodiment a surfactant containing substance may comprise an aryloxy group. In one embodiment a surfactant containing substance may comprise an alkoxy group.

In one embodiment a surfactant containing substance may comprise a pyrrole group. In one embodiment a surfactant containing substance may comprise a pyrroline group. In one embodiment a surfactant containing substance may comprise a pyrrolidine group. In one embodiment a surfactant containing substance may comprise a pyrrolidinium group. In one embodiment a surfactant containing substance may comprise an indole group. In one embodiment a surfactant containing substance may comprise a pyrrolizidine group. In one embodiment a surfactant containing substance may comprise a piperidine group. In one embodiment a surfactant containing substance may comprise an oxazole group. In one embodiment a surfactant containing substance may comprise an isoxazole group. In one embodiment a surfactant containing substance may comprise a thiazole group. In one embodiment a surfactant containing substance may comprise an imidazole or imidazolium group. In one embodiment a surfactant containing substance may comprise a tropane group. In one embodiment a surfactant containing substance may comprise one or more pyridine or pyridinium group.

In one embodiment a surfactant containing substance may not comprise an aryl group. In one embodiment a surfactant containing substance may not comprise a phenyl group. In one embodiment a surfactant containing substance may not comprise a benzene ring. In one embodiment a surfactant containing substance may not comprise a phenol. In one embodiment a surfactant containing substance may not comprise an aryloxy group. In one embodiment a surfactant containing substance may not comprise an alkoxy group.

In one embodiment a surfactant containing substance may comprise one or more quaternary ammonium. In one embodiment a surfactant containing substance may comprise one or more tertiary amine. In one embodiment a surfactant containing substance may comprise one or more tertiary sulfonium. In one embodiment a surfactant containing substance may comprise one or more quaternary phosphonium.

In one embodiment a surfactant containing substance may comprise one or more quaternary ammonium cation or cationic moiety. In one embodiment a surfactant containing substance may comprise one or more tertiary sulfonium cation or cationic moiety. In one embodiment a surfactant containing substance may comprise one or more quaternary phosphonium cation or cationic moiety.

In one embodiment a surfactant containing substance may comprise one or more aliphatic quaternary ammonium. In one embodiment a surfactant containing substance may comprise one or more aliphatic tertiary amine. In one embodiment a surfactant containing substance may comprise one or more quaternary ammonium nitrogen heterocycle. In one embodiment a surfactant containing substance may comprise one or more quaternary ammonium aromatic nitrogen heterocycle. In one embodiment a surfactant containing substance may comprise one or more five membered quaternary ammonium nitrogen heterocycle. In one embodiment a surfactant containing substance may comprise one or more six membered quaternary ammonium nitrogen heterocycle. In one embodiment a surfactant containing substance may comprise one or more five membered aromatic quaternary ammonium nitrogen heterocycle. In one embodiment a surfactant containing substance may comprise one or more six membered aromatic quaternary ammonium nitrogen heterocycle.

In one embodiment a surfactant containing substance may comprise one or more cation or cationic moiety. In one embodiment a surfactant containing substance may comprise one or more anion or anionic moiety. In one embodiment a surfactant containing substance may comprise one or more zwitterion or zwitterionic moiety. In one embodiment a surfactant containing substance may be zwitterionic.

In one embodiment a surfactant containing substance may comprise one or more betaine. In one embodiment a surfactant containing substance may comprise one or more carboxybetaine. In one embodiment a surfactant containing substance may comprise one or more sulfobetaine.

In one embodiment a surfactant containing substance may comprise one or more quaternary ammonium cation that may be at least part of one or more nitrogen heterocycle or aromatic nitrogen heterocycle, such as a pyridinium, pyrrolidinium, imidazolium, piperidinium, indolium, pyrimidinium, or purinium group as non-limiting examples.

In one embodiment a surfactant containing substance may comprise one or more nitrogen heterocycle or aromatic nitrogen heterocycle, such as a pyridinium, pyrrolidinium, imidazolium, piperidinium, indolium, pyrimidinium, or purinium group as non-limiting examples.

In one embodiment a surfactant containing substance may comprise one or more carboxylate group. In one embodiment a surfactant containing substance may comprise one or more carboxylate ester. In one embodiment a surfactant containing substance may comprise one or more sulfonate group. In one embodiment a surfactant containing substance may comprise one or more organosulfate group.

In one embodiment a surfactant containing substance may comprise one or more carboxylate anion or anionic moiety. In one embodiment a surfactant containing substance may comprise one or more sulfonate anion or anionic moiety. In one embodiment a surfactant containing substance may comprise one or more organosulfate anion or anionic moiety.

In one embodiment a surfactant containing substance may not comprise a pyrrole group. In one embodiment a surfactant containing substance may not comprise a pyrroline group. In one embodiment a surfactant containing substance may not comprise a pyrrolidine group. In one embodiment a surfactant containing substance may not comprise a pyrrolidinium group. In one embodiment a surfactant containing substance may not comprise an indole group. In one embodiment a surfactant containing substance may not comprise a pyrrolizidine group. In one embodiment a surfactant containing substance may not comprise a piperidine group. In one embodiment a surfactant containing substance may not comprise an oxazole group. In one embodiment a surfactant containing substance may not comprise an isoxazole group. In one embodiment a surfactant containing substance may not comprise a thiazole group. In one embodiment a surfactant containing substance may not comprise an imidazole or imidazolium group. In one embodiment a surfactant containing substance may not comprise a tropane group. In one embodiment a surfactant containing substance may not comprise one or more pyridine or pyridinium group.

In one embodiment a surfactant containing substance may comprise a tail group (represented by a TG group). In one embodiment a surfactant containing substance may comprise a head group (represented by an HG group). In one embodiment a head group may be comprised of one or more anionic group (represented by an AG group) or one or more cationic group (represented by a CG group). In one embodiment a head group may be comprised of both an anionic group and a cationic group. In one embodiment a head group may be zwitterionic. In one embodiment a head group may be cationic. In one embodiment a head group may be anionic.

In one embodiment a surfactant containing substance may comprise the following formula: (TG)-(HG)

Wherein, TG is a tail group comprised of at least 6 carbons atoms, covalently bonded to a head group (HG).

Furthermore, HG may be comprised of one or more of the following formulas: (CG), or (AG), or (CG-AG), or (AG-CG), or (CG-V-AG), or (AG-V-CG), or (CG-V), or (AG-V)

Wherein a head group may be comprised of either an anionic group (represented by an AG group) or a cationic group (represented by a CG group), or both a cationic group and an anionic group, wherein a CG group and an AG group may be covalently bonded directly, such as AG-CG or CG-AG as non-limiting examples, or a CG group and an AG group may be connected by a V group, such as CG-V-AG or AG-V-CG as non-limiting examples. Furthermore, V is a V group, wherein a V group is comprised of at least one carbon atom.

In one embodiment AG may comprise one or more of the following: a carboxylate group, or a sulfonate group, or a organosulfate group, or a phosphonate group, or a phosphate ester, or an organophosphate group.

In one embodiment a CG group may comprise a nitrogen heterocycle. In one embodiment a CG group may comprise a six membered nitrogen heterocycle. In one embodiment a CG group may comprise a five membered nitrogen heterocycle. In one embodiment a CG group may comprise an aromatic nitrogen heterocycle. In one embodiment a CG group may comprise an aromatic six membered nitrogen heterocycle. In one embodiment a CG group may comprise an aromatic five membered nitrogen heterocycle.

In one embodiment CG may comprise one or more of the following: a quaternary ammonium, or a tertiary amine, or a tertiary sulfonium, or a pyridinium, or an imidazole, or an imidazolium, or a pyrrole, or a pyridine, or a pyridinium, or a pyrrolidine, or a pyrrolidinium, or a pyrrole, or a pyrazole, or a piperidine, or a piperidinium, or cationic ammonium moiety, or cationic sulfonium moiety.

In one embodiment a V group may be absent. In one embodiment a surfactant containing substance may comprise one or more V group wherein one or more AG or one or more CG may be absent, such as in the formula TG-CG-V as a non-limiting example. In one embodiment a surfactant containing substance may comprise one or more AG or one or more CG wherein a V group may be absent, such as in the formula TG-CG-AG as a non-limiting example.

In one embodiment a surfactant containing substance may not comprise a V group. In one embodiment a surfactant containing substance may not comprise AG or CG. In one embodiment a surfactant containing substance may comprise a V group without AG or without CG. In one embodiment a surfactant containing substance may comprise a V group with AG. In one embodiment a surfactant containing substance may comprise a V group with CG. In one embodiment AG may be covalently bonded to CG with a V group. In one embodiment a CG group may be covalently bonded to an AG group without a V group. In one embodiment a CG group may be covalently bonded to a V group. In one embodiment an AG group may be covalently bonded to a V group.

In one embodiment a CG group may not be covalently bonded to a V group. In one embodiment an AG group may not be covalently bonded to a V group.

In one embodiment a V group may be comprised of at least one or more carbon atoms. Wherein, V may be comprised of a C1-35 alkyl, alkenyl, alkynyl, aryl, or aralkyl group, each of which may contain up to 20 heteroatoms. In another embodiment a V group may be comprised of C1-30 alkyl, alkenyl, alkynyl, aryl, or aralkyl group, each of which may contain up to 16 heteroatoms. In another embodiment a V group may be comprised of C1-24 alkyl, alkenyl, alkynyl, aryl, or aralkyl group, each of which may contain up to 12 heteroatoms. In another embodiment a V group may be comprised of C1-20 alkyl, alkenyl, alkynyl, aryl, or aralkyl group, each of which may contain up to 10 heteroatoms. In another embodiment a V group may be comprised of C1-16 alkyl, alkenyl, alkynyl, aryl, or aralkyl group, each of which may contain up to 8 heteroatoms. In another embodiment a V group may be comprised of C1-12 alkyl, alkenyl, alkynyl, aryl, or aralkyl group, each of which may contain up to 6 heteroatoms. In another embodiment a V group may be comprised of C1-12 alkyl, alkenyl, alkynyl, aryl, or aralkyl group, each of which may contain up to 4 heteroatoms. In another embodiment a V group may be comprised of C1-8 alkyl, alkenyl, alkynyl, aryl, or aralkyl group, each of which may contain up to 4 heteroatoms. In another embodiment a V group may be comprised of C1-8 alkyl, alkenyl, alkynyl, aryl, or aralkyl group, each of which may contain up to 3 heteroatoms. In another embodiment a V group may be comprised of C1-6 alkyl, alkenyl, alkynyl, aryl, or aralkyl group, each of which may contain up to 3 heteroatoms. In another embodiment a V group may be comprised of C1-6 alkyl, alkenyl, alkynyl, aryl, or aralkyl group, each of which may contain up to 2 heteroatoms. In another embodiment a V group may be comprised of C1-4 alkyl, alkenyl, or alkynyl group, each of which may contain up to 2 heteroatoms.

In one embodiment a TG group may be comprised of at least one or more six carbon atoms. Wherein, TG may be comprised of a C6-35 alkyl, alkenyl, alkynyl, aryl, or aralkyl group, each of which may contain up to 20 heteroatoms. In another embodiment a TG group may be comprised of C6-30 alkyl, alkenyl, alkynyl, aryl, or aralkyl group, each of which may contain up to 16 heteroatoms. In another embodiment a TG group may be comprised of C6-24 alkyl, alkenyl, alkynyl, aryl, or aralkyl group, each of which may contain up to 12 heteroatoms. In another embodiment a TG group may be comprised of C6-20 alkyl, alkenyl, alkynyl, aryl, or aralkyl group, each of which may contain up to 10 heteroatoms. In another embodiment a TG group may be comprised of C6-20 alkyl, alkenyl, alkynyl, aryl, or aralkyl group, each of which may contain up to 6 heteroatoms. In another embodiment a TG group may be comprised of C6-20 alkyl, alkenyl, alkynyl, aryl, or aralkyl group, each of which may contain up to 4 heteroatoms. In another embodiment a TG group may be comprised of C6-16 alkyl, alkenyl, alkynyl, aryl, or aralkyl group, each of which may contain up to 8 heteroatoms. In another embodiment a TG group may be comprised of C6-16 alkyl, alkenyl, alkynyl, aryl, or aralkyl group, each of which may contain up to 6 heteroatoms. In another embodiment a TG group may be comprised of C6-16 alkyl, alkenyl, alkynyl, aryl, or aralkyl group, each of which may contain up to 4 heteroatoms. In another embodiment a TG group may be comprised of C6-12 alkyl, alkenyl, alkynyl, aryl, or aralkyl group, each of which may contain up to 6 heteroatoms. In another embodiment a TG group may be comprised of C6-12 alkyl, alkenyl, alkynyl, aryl, or aralkyl group, each of which may contain up to 4 heteroatoms. In another embodiment a TG group may be comprised of C6-12 alkyl, alkenyl, alkynyl, aryl, or aralkyl group, each of which may contain up to 2 heteroatoms. In another embodiment a TG group may be comprised of C6-8 alkyl, alkenyl, alkynyl, aryl, or aralkyl group, each of which may contain up to 4 heteroatoms. In another embodiment a TG group may be comprised of C6-8 alkyl, alkenyl, alkynyl, aryl, or aralkyl group, each of which may contain up to 2 heteroatoms.

In one embodiment a TG group may be aliphatic. In one embodiment a TG group may not comprise an aryl group or an aralkyl group.

In one embodiment a TG group may be comprised of at least one or more six carbon atoms. Wherein, TG may be comprised of a C6-35 alkyl, alkenyl, or alkynyl group, each of which may contain up to 20 heteroatoms. In another embodiment a TG group may be comprised of C6-30 alkyl, alkenyl, or alkynyl group, each of which may contain up to 16 heteroatoms. In another embodiment a TG group may be comprised of C6-24 alkyl, alkenyl, or alkynyl group, each of which may contain up to 12 heteroatoms. In another embodiment a TG group may be comprised of C6-20 alkyl, alkenyl, or alkynyl group, each of which may contain up to 10 heteroatoms. In another embodiment a TG group may be comprised of C6-20 alkyl, alkenyl, or alkynyl group, each of which may contain up to 6 heteroatoms. In another embodiment a TG group may be comprised of C6-20 alkyl, alkenyl, or alkynyl group, each of which may contain up to 4 heteroatoms. In another embodiment a TG group may be comprised of C6-16 alkyl, alkenyl, or alkynyl group, each of which may contain up to 8 heteroatoms. In another embodiment a TG group may be comprised of C6-16 alkyl, alkenyl, or alkynyl group, each of which may contain up to 6 heteroatoms. In another embodiment a TG group may be comprised of C6-16 alkyl, alkenyl, or alkynyl group, each of which may contain up to 4 heteroatoms. In another embodiment a TG group may be comprised of C6-12 alkyl, alkenyl, or alkynyl group, each of which may contain up to 6 heteroatoms. In another embodiment a TG group may be comprised of C6-12 alkyl, alkenyl, or alkynyl group, each of which may contain up to 4 heteroatoms. In another embodiment a TG group may be comprised of C6-12 alkyl, alkenyl, or alkynyl group, each of which may contain up to 2 heteroatoms. In another embodiment a TG group may be comprised of C6-8 alkyl, alkenyl, or alkynyl group, each of which may contain up to 4 heteroatoms. In another embodiment a TG group may be comprised of C6-8 alkyl, alkenyl, or alkynyl group, each of which may contain up to 2 heteroatoms.

In one embodiment a TG group may be comprised of one or more cholic acid or cholate derivative. In one embodiment a TG group may be comprised of one or more cholate ester derivative.

In one embodiment a surfactant containing substance may be hydroxylated. In one embodiment a surfactant containing substance may comprise one or more hydroxy groups. In one embodiment a surfactant containing substance may comprise 1 or more hydroxy groups, 2 or more hydroxy groups, 3 or more hydroxy groups, 4 or more hydroxy groups, 5 or more hydroxy groups, or more hydroxy groups, or 50 or more hydroxy groups. In one embodiment a surfactant containing substance may comprise between about 1-100 hydroxy groups, or between about 1-50 hydroxy groups, or between about 1-20 hydroxy groups, or between about 1-10 hydroxy groups, or between about 1-5 hydroxy groups, or between about 1-3 hydroxy groups, or between about 2-100 hydroxy groups, or between about 2-50 hydroxy groups, or between about 2-20 hydroxy groups, or between about 2-10 hydroxy groups, or between about 2-5 hydroxy groups, or between about 2-3 hydroxy groups.

In one embodiment an HG group may be hydroxylated. In one embodiment an HG group may comprise one or more hydroxy groups. In one embodiment an HG group may comprise 1 or more hydroxy groups, 2 or more hydroxy groups, 3 or more hydroxy groups, 4 or more hydroxy groups, 5 or more hydroxy groups, 10 or more hydroxy groups, or 50 or more hydroxy groups. In one embodiment an HG group may comprise between about 1-100 hydroxy groups, or between about 1-50 hydroxy groups, or between about 1-20 hydroxy groups, or between about 1-10 hydroxy groups, or between about 1-5 hydroxy groups, or between about 1-3 hydroxy groups, or between about 2-100 hydroxy groups, or between about 2-50 hydroxy groups, or between about 2-20 hydroxy groups, or between about 2-10 hydroxy groups, or between about 2-5 hydroxy groups, or between about 2-3 hydroxy groups.

In one embodiment an AG group or CG group may be hydroxylated. In one embodiment an AG group or CG group may comprise one or more hydroxy groups. In one embodiment an AG group or CG group may comprise 1 or more hydroxy groups, 2 or more hydroxy groups, 3 or more hydroxy groups, 4 or more hydroxy groups, 5 or more hydroxy groups, 10 or more hydroxy groups, or 50 or more hydroxy groups. In one embodiment an AG group or CG group may comprise between about 1-100 hydroxy groups, or between about 1-50 hydroxy groups, or between about 1-20 hydroxy groups, or between about 1-10 hydroxy groups, or between about 1-5 hydroxy groups, or between about 1-3 hydroxy groups, or between about 2-100 hydroxy groups, or between about 2-50 hydroxy groups, or between about 2-20 hydroxy groups, or between about 2-10 hydroxy groups, or between about 2-5 hydroxy groups, or between about 2-3 hydroxy groups.

In one embodiment an V group may be hydroxylated. In one embodiment an V group may comprise one or more hydroxy groups. In one embodiment an V group may comprise 1 or more hydroxy groups, 2 or more hydroxy groups, 3 or more hydroxy groups, 4 or more hydroxy groups, or more hydroxy groups, 10 or more hydroxy groups, or 50 or more hydroxy groups. In one embodiment an V group may comprise between about 1-100 hydroxy groups, or between about 1-50 hydroxy groups, or between about 1-20 hydroxy groups, or between about 1-10 hydroxy groups, or between about 1-5 hydroxy groups, or between about 1-3 hydroxy groups, or between about 2-100 hydroxy groups, or between about 2-50 hydroxy groups, or between about 2-20 hydroxy groups, or between about 2-10 hydroxy groups, or between about 2-5 hydroxy groups, or between about 2-3 hydroxy groups.

In one embodiment a TG group may be hydroxylated. In one embodiment a TG group may comprise one or more hydroxy groups. In one embodiment a TG group may comprise 1 or more hydroxy groups, 2 or more hydroxy groups, 3 or more hydroxy groups, 4 or more hydroxy groups, or more hydroxy groups, 10 or more hydroxy groups, or 50 or more hydroxy groups. In one embodiment a TG group may comprise between about 1-100 hydroxy groups, or between about 1-50 hydroxy groups, or between about 1-20 hydroxy groups, or between about 1-10 hydroxy groups, or between about 1-5 hydroxy groups, or between about 1-3 hydroxy groups, or between about 2-100 hydroxy groups, or between about 2-50 hydroxy groups, or between about 2-20 hydroxy groups, or between about 2-10 hydroxy groups, or between about 2-5 hydroxy groups, or between about 2-3 hydroxy groups.

In one embodiment a surfactant containing substance may comprise one or more amide groups. In one embodiment a surfactant containing substance may comprise 1 or more amide groups, 2 or more amide groups, 3 or more amide groups, 4 or more amide groups, 5 or more amide groups, 10 or more amide groups, or 50 or more amide groups. In one embodiment a surfactant containing substance may comprise between about 1-100 amide groups, or between about 1-50 amide groups, or between about 1-20 amide groups, or between about 1-10 amide groups, or between about 1-5 amide groups, or between about 1-3 amide groups, or between about 2-100 amide groups, or between about 2-50 amide groups, or between about 2-20 amide groups, or between about 2-10 amide groups, or between about 2-5 amide groups, or between about 2-3 amide groups.

In one embodiment an HG group may comprise one or more amide groups. In one embodiment an HG group may comprise 1 or more amide groups, 2 or more amide groups, 3 or more amide groups, 4 or more amide groups, 5 or more amide groups, 10 or more amide groups, or 50 or more amide groups. In one embodiment an HG group may comprise between about 1-100 amide groups, or between about 1-50 amide groups, or between about 1-20 amide groups, or between about 1-10 amide groups, or between about 1-5 amide groups, or between about 1-3 amide groups, or between about 2-100 amide groups, or between about 2-50 amide groups, or between about 2-20 amide groups, or between about 2-10 amide groups, or between about 2-5 amide groups, or between about 2-3 amide groups.

In one embodiment an AG group or CG group may comprise one or more amide groups. In one embodiment an AG group or CG group may comprise 1 or more amide groups, 2 or more amide groups, 3 or more amide groups, 4 or more amide groups, 5 or more amide groups, 10 or more amide groups, or 50 or more amide groups. In one embodiment an AG group or CG group may comprise between about 1-100 amide groups, or between about 1-50 amide groups, or between about 1-20 amide groups, or between about 1-10 amide groups, or between about 1-5 amide groups, or between about 1-3 amide groups, or between about 2-100 amide groups, or between about 2-50 amide groups, or between about 2-20 amide groups, or between about 2-10 amide groups, or between about 2-5 amide groups, or between about 2-3 amide groups.

In one embodiment a V group may comprise one or more amide groups. In one embodiment a V group may comprise 1 or more amide groups, 2 or more amide groups, 3 or more amide groups, 4 or more amide groups, 5 or more amide groups, 10 or more amide groups, or 50 or more amide groups. In one embodiment a V group may comprise between about 1-100 amide groups, or between about 1-50 amide groups, or between about 1-20 amide groups, or between about 1-10 amide groups, or between about 1-5 amide groups, or between about 1-3 amide groups, or between about 2-100 amide groups, or between about 2-50 amide groups, or between about 2-amide groups, or between about 2-10 amide groups, or between about 2-5 amide groups, or between about 2-3 amide groups.

In one embodiment a TG group may comprise one or more amide groups. In one embodiment a TG group may comprise 1 or more amide groups, 2 or more amide groups, 3 or more amide groups, 4 or more amide groups, 5 or more amide groups, 10 or more amide groups, or 50 or more amide groups. In one embodiment a TG group may comprise between about 1-100 amide groups, or between about 1-50 amide groups, or between about 1-20 amide groups, or between about 1-10 amide groups, or between about 1-5 amide groups, or between about 1-3 amide groups, or between about 2-100 amide groups, or between about 2-50 amide groups, or between about 2-20 amide groups, or between about 2-10 amide groups, or between about 2-5 amide groups, or between about 2-3 amide groups.

Embodiments of the present invention comprising a surfactant containing substance may comprise a V group wherein a V group may comprise one or more nitrogen atom or one or more oxygen atom or one or more sulfur atom or one or more phosphorus atom. Embodiments of the present invention comprising a surfactant containing substance may comprise a V group wherein a V group may comprise, but is not limited to, one or more of the following groups: hydroxy groups, alcohols, alkyl groups, alkenyl groups, alkynyl groups, aryl groups, aralkyl groups, ether groups, ethers, carboxamide groups, amides, carboalkoxy groups, esters, carbonate esters, or carboxylate esters.

Embodiments of the present invention comprising a surfactant containing substance may comprise a TG group wherein a TG group may comprise one or more nitrogen atom or one or more oxygen atom or one or more sulfur atom or one or more phosphorus atom. Embodiments of the present invention comprising a surfactant containing substance may comprise a TG group wherein a TG group may comprise, but is not limited to, one or more of the following groups: hydroxy groups, alcohols, alkyl groups, alkenyl groups, alkynyl groups, aryl groups, aralkyl groups, ether groups, ethers, carboxamide groups, amides, carboalkoxy groups, esters, carbonate esters, or carboxylate esters.

A non-limiting example of a surfactant containing substance comprising the formula (TG)-(HG) is trimethyloctylammonium, wherein HG is equal to CG, wherein CG is comprised of a quaternary ammonium bonded to four methyl groups and is covalently bonded to TG wherein TG is comprised of an aliphatic 7 carbon alkane.

A non-limiting example of a surfactant containing substance comprising the formula (TG)-(HG) is stearate, wherein HG is equal to AG, wherein AG is comprised of a carboxylate group covalently bonded to TG wherein TG is comprised of an aliphatic 17 carbon alkane.

A non-limiting example of a surfactant containing substance comprising the formula (TG)-(HG) is lauroamphoacetate, wherein HG is equal to (CG-V-AG), wherein AG is comprised of a carboxylate group, CG is comprised of a imidazolium group and V is comprised of at least one carbon atom covalently bonding AG to CG. Finally, TG is comprised of an aliphatic 11 carbon alkane covalently bonded to the CG of HG.

A non-limiting example of a surfactant containing substance comprising the formula (TG)-(HG) is cetylpyridinium, wherein HG is equal to CG, wherein CG is comprised of a pyridinium group covalently bonded to TG wherein TG is comprised of an aliphatic 16 carbon alkane.

In one embodiment a V group may comprise a heterocyclic ring structure with a covalently bonded nitrogen atom, phosphorus atom, or sulfur atom.

In one embodiment a V group may comprise a ring structure, such as a five membered or six membered ring structure as non-limiting examples. In another embodiment a V group may comprise at least part of a ring structure, such as a five membered or six membered ring structure as non-limiting examples.

In one embodiment a V group may comprise an aromatic ring structure, such as an aromatic five membered or six membered ring structure as non-limiting examples. In another embodiment a V group may comprise at least part of an aromatic ring structure, such as an aromatic five membered or six membered ring structure as non-limiting examples.

In one embodiment a V group may not comprise a ring structure. In another embodiment a V group may not comprise at least part of a ring structure. In one embodiment a V group may not comprise an aromatic ring structure. In another embodiment a V group may not comprise at least part of an aromatic ring structure.

A non-limiting example of a cationic surfactant containing substance that may be used is trimethyloctylammonium (also known as octyltrimethylammonium or n-octyltrimethylammonium), such as trimethyloctylammonium bromide as a non-limiting example, wherein trimethyloctylammonium may be substituted for or used in combination with one or more surfactant containing substance as described herein. One embodiment of the present invention may include combinations of substances comprising trimethyloctylammonium and another embodiment may also comprise at least one RNA substance. Other embodiments may include combinations of substances comprising trimethyloctylammonium wherein the concentration of trimethyloctylammonium may be one or more concentration in the stabilizing substance weight percent concentration list or in the stabilizing substance molar concentration range list or in the stabilizing substance molar concentration list.

A non-limiting example of a cationic surfactant containing substance, comprising an imidazolium group, that may be used is 1-decyl-3-methylimidazolium, such as 1-decyl-3-methylimidazolium chloride as a non-limiting example, wherein 1-decyl-3-methylimidazolium may be substituted for or used in combination with one or more surfactant containing substance as described herein. One embodiment of the present invention may include combinations of substances comprising 1-decyl-3-methylimidazolium and another embodiment may also comprise at least one RNA substance. Other embodiments may include combinations of substances comprising 1-decyl-3-methylimidazolium wherein the concentration of 1-decyl-3-methylimidazolium may be one or more concentration in the stabilizing substance weight percent concentration list or in the stabilizing substance molar concentration range list or in the stabilizing substance molar concentration list.

A non-limiting example of a cationic surfactant containing substance that may be used is hexadecyltrimethylammonium, such as hexadecyltrimethylammonium chloride as a non-limiting example, wherein hexadecyltrimethylammonium may be substituted for or used in combination with one or more surfactant containing substance as described herein. One embodiment of the present invention may include combinations of substances comprising hexadecyltrimethylammonium and another embodiment may also comprise at least one RNA substance. Other embodiments may include combinations of substances comprising hexadecyltrimethylammonium wherein the concentration of hexadecyltrimethylammonium may be one or more concentration in the stabilizing substance weight percent concentration list or in the stabilizing substance molar concentration range list or in the stabilizing substance molar concentration list.

A non-limiting example of a cationic surfactant containing substance, comprised of a pyridinium group, that may be used is cetylpyridinium, such as cetylpyridinium chloride as a non-limiting example, wherein cetylpyridinium may be substituted for or used in combination with one or more surfactant containing substance as described herein. One embodiment of the present invention may include combinations of substances comprising cetylpyridinium and another embodiment may also comprise at least one RNA substance. Other embodiments may include combinations of substances comprising cetylpyridinium wherein the concentration of cetylpyridinium may be one or more concentration in the stabilizing substance weight percent concentration list or in the stabilizing substance molar concentration range list or in the stabilizing substance molar concentration list.

A non-limiting example of a cationic surfactant containing substance, comprised of an aryl group, that may be used is stearalkonium, such as stearalkonium chloride as a non-limiting example, wherein stearalkonium may be substituted for or used in combination with one or more surfactant containing substance as described herein. One embodiment of the present invention may include combinations of substances comprising stearalkonium and another embodiment may also comprise at least one RNA substance. Other embodiments may include combinations of substances comprising stearalkonium wherein the concentration of stearalkonium may be one or more concentration in the stabilizing substance weight percent concentration list or in the stabilizing substance molar concentration range list or in the stabilizing substance molar concentration list.

A non-limiting example of a cationic surfactant containing substance that may be used is dimethyldioctadecylammonium (also known as distearyldimethylammonium), such as dimethyldioctadecylammonium chloride as a non-limiting example, wherein dimethyldioctadecylammonium may be substituted for or used in combination with one or more surfactant containing substance as described herein. One embodiment of the present invention may include combinations of substances comprising dimethyldioctadecylammonium and another embodiment may also comprise at least one RNA substance. Other embodiments may include combinations of substances comprising dimethyldioctadecylammonium wherein the concentration of dimethyldioctadecylammonium may be one or more concentration in the stabilizing substance weight percent concentration list or in the stabilizing substance molar concentration range list or in the stabilizing substance molar concentration list.

A non-limiting example of a cationic surfactant containing substance that may be used is didecyldimethylammonium, such as didecyldimethylammonium chloride as a non-limiting example, wherein didecyldimethylammonium may be substituted for or used in combination with one or more surfactant containing substance as described herein. One embodiment of the present invention may include combinations of substances comprising didecyldimethylammonium and another embodiment may also comprise at least one RNA substance. Other embodiments may include combinations of substances comprising didecyldimethylammonium wherein the concentration of didecyldimethylammonium may be one or more concentration in the stabilizing substance weight percent concentration list or in the stabilizing substance molar concentration range list or in the stabilizing substance molar concentration list.

A non-limiting example of an anionic surfactant containing substance that may be used is dodecyl sulfate, such as sodium dodecyl sulfate (SDS) as a non-limiting example, wherein dodecyl sulfate may be substituted for or used in combination with one or more surfactant containing substance as described herein. One embodiment of the present invention may include combinations of substances comprising dodecyl sulfate and another embodiment may also comprise at least one RNA substance. Other embodiments may include combinations of substances comprising dodecyl sulfate wherein the concentration of dodecyl sulfate may be one or more concentration in the stabilizing substance weight percent concentration list or in the stabilizing substance molar concentration range list or in the stabilizing substance molar concentration list.

A non-limiting example of an anionic surfactant containing substance that may be used is stearate, such as sodium stearate as a non-limiting example, wherein stearate may be substituted for or used in combination with one or more surfactant containing substance as described herein. One embodiment of the present invention may include combinations of substances comprising stearate and another embodiment may also comprise at least one RNA substance. Other embodiments may include combinations of substances comprising stearate wherein the concentration of stearate may be one or more concentration in the stabilizing substance weight percent concentration list or in the stabilizing substance molar concentration range list or in the stabilizing substance molar concentration list.

A non-limiting example of a zwitterionic surfactant containing substance that may be used is lauroamphoacetate, such as sodium lauroamphoacetate, as a non-limiting example, wherein lauroamphoacetate may be substituted for or used in combination with one or more surfactant containing substance as described herein. One embodiment of the present invention may include combinations of substances comprising lauroamphoacetate and another embodiment may also comprise at least one RNA substance. Other embodiments may include combinations of substances comprising lauroamphoacetate wherein the concentration of lauroamphoacetate may be one or more concentration in the stabilizing substance weight percent concentration list or in the stabilizing substance molar concentration range list or in the stabilizing substance molar concentration list.

A non-limiting example of a zwitterionic surfactant containing substance that may be used is lauramidopropyl betaine, such as lauramidopropyl betaine as a non-limiting example, wherein lauramidopropyl betaine may be substituted for or used in combination with one or more surfactant containing substance as described herein. One embodiment of the present invention may include combinations of substances comprising lauramidopropyl betaine and another embodiment may also comprise at least one RNA substance. Other embodiments may include combinations of substances comprising lauramidopropyl betaine wherein the concentration of lauramidopropyl betaine may be one or more concentration in the stabilizing substance weight percent concentration list or in the stabilizing substance molar concentration range list or in the stabilizing substance molar concentration list.

A non-limiting example of a zwitterionic surfactant containing substance that may be used is hydroxysultaine, such as cocamidopropyl hydroxysultaine as a non-limiting example, wherein hydroxysultaine may be substituted for or used in combination with one or more surfactant containing substance as described herein. One embodiment of the present invention may include combinations of substances comprising hydroxysultaine and another embodiment may also comprise at least one RNA substance. Other embodiments may include combinations of substances comprising hydroxysultaine wherein the concentration of hydroxysultaine may be one or more concentration in the stabilizing substance weight percent concentration list or in the stabilizing substance molar concentration range list or in the stabilizing substance molar concentration list.

For conciseness, the following list of surfactant containing substances is herein referred to as the surfactant containing substance list wherein one or more of the following substances may be substituted for or used in combination with one or more surfactant containing substance as described herein.

In other embodiments, other surfactant containing substances that may be substituted for or used in combination with one or more surfactant containing substance as described herein may comprise one or more of the following, including but not limited to: trimethyloctylammonium, 1-decyl-3-methylimidazolium, hexadecyltrimethylammonium, cetylpyridinium, stearalkonium, dimethyldioctadecylammonium, didecyldimethylammonium, dodecyl sulfate, stearate, lauroamphoacetate, lauramidopropyl betaine, hydroxysultaines, cocamidopropyl hydroxysultaine, cocamidopropyl betaine, lauramidopropyl betaine, CHAPS detergent, or salts, or derivatives, or combinations thereof.

Embodiments of the present invention may include combinations comprising one or more of the substances from the surfactant containing substance list substituted for or used in combination with one or more surfactant containing substance or one or more RNA stabilizing substance, such as an aprotic substance as a non-limiting example, and other embodiments may also comprise at least one RNA substance.

Other embodiments may include combinations of substances comprising one or more surfactant containing substance selected from the surfactant containing substance list wherein the concentration of one or more substances may be one or more concentration in the stabilizing substance weight percent concentration list or in the stabilizing substance molar concentration range list or in the stabilizing substance molar concentration list.

In one embodiment, a surfactant containing substance may have a molecular weight between about 50-5,000 daltons. In one embodiment, a surfactant containing substance may have a molecular weight between about 50-2,000 daltons. In one embodiment, a surfactant containing substance may have a molecular weight between about 50-1,000 daltons. In one embodiment, a surfactant containing substance may have a molecular weight between about 50-900 daltons. In one embodiment, a surfactant containing substance may have a molecular weight between about 50-800 daltons. In one embodiment, a surfactant containing substance may have a molecular weight between about 50-700 daltons. In one embodiment, a surfactant containing substance may have a molecular weight between about 50-600 daltons. In one embodiment, a surfactant containing substance may have a molecular weight between about 50-500 daltons. In one embodiment, a surfactant containing substance may have a molecular weight between about 50-400 daltons. In one embodiment, a surfactant containing substance may have a molecular weight between about 50-300 daltons.

In one embodiment, a surfactant containing substance may have a molecular weight between about 100-5,000 daltons. In one embodiment, a surfactant containing substance may have a molecular weight between about 100-2,000 daltons. In one embodiment, a surfactant containing substance may have a molecular weight between about 100-1,000 daltons. In one embodiment, a surfactant containing substance may have a molecular weight between about 100-900 daltons. In one embodiment, a surfactant containing substance may have a molecular weight between about 100-800 daltons. In one embodiment, a surfactant containing substance may have a molecular weight between about 100-700 daltons. In one embodiment, a surfactant containing substance may have a molecular weight between about 100-600 daltons. In one embodiment, a surfactant containing substance may have a molecular weight between about 100-500 daltons. In one embodiment, a surfactant containing substance may have a molecular weight between about 100-400 daltons. In one embodiment, a surfactant containing substance may have a molecular weight between about 100-300 daltons.

One embodiment of the present invention is the method whereby one or more surfactant containing substance may be combined, such as by mixing, with at least one or more RNA substance to produce a mixture comprising at least one or more surfactant containing substance and at least one or more RNA substance. As a non-limiting example one or more RNA substance may be mixed with one or more surfactant containing substance.

Another embodiment of the present invention is the method whereby one or more surfactant containing substance may be combined, such as by mixing, with at least one or more RNA substance to produce a mixture comprising at least one or more RNA substance and at least one or more surfactant containing substance at one or more of the RNA stabilizing substance concentrations within the stabilizing substance molar concentration range list or in the stabilizing substance molar concentration list or the stabilizing substance weight percent concentration list as described herein. These same methods may be used to combine one or more other substances, including, but not limited to one or more cellular uptake agents or one or more additional RNA stabilizing substances, with one or more RNA substances and one or more surfactant containing substances to produce a mixture comprising one or more RNA substance, one or more surfactant containing substance, and one or more cellular uptake agent or one or more additional RNA stabilizing substance.

One embodiment of the present invention is the method whereby one or more surfactant containing substance may be combined, such as by mixing, with at least one or more RNA substance to produce a composition comprising at least one or more RNA substance and at least one or more surfactant containing substance. Another embodiment of the present invention is the method whereby one or more surfactant containing substance may be combined, such as by mixing, with one or more RNA substance to produce a composition comprising at least one or more RNA substance and at least one or more surfactant containing substance at one or more of the RNA stabilizing substance concentrations within the stabilizing substance molar concentration range list or in the stabilizing substance molar concentration list or stabilizing substance weight percent concentration list as described herein. These same methods may be used to combine one or more other substances, including, but not limited to one or more cellular uptake agents or one or more additional RNA stabilizing substance, with one or more RNA substance and one or more surfactant containing substance to produce a composition comprising one or more RNA substance, one or more surfactant containing substance, and one or more cellular uptake agent or one or more additional RNA stabilizing substance.

In one embodiment a surfactant containing substance may comprise a ring structure. In another embodiment a surfactant containing substance may comprise a heterocyclic ring structure. In one embodiment a surfactant containing substance may comprise an aromatic ring structure. In another embodiment a surfactant containing substance may comprise an aromatic heterocyclic ring structure.

In one embodiment a surfactant containing substance may comprise a cationic moiety at about physiologic pH. In one embodiment a surfactant containing substance may comprise an anionic moiety at about physiologic pH. In one embodiment a surfactant containing substance may comprise a zwitterion at about physiologic pH.

In one embodiment a surfactant containing substance may comprise a detergent. In one embodiment a surfactant containing substance may comprise an anionic detergent. In one embodiment a surfactant containing substance may comprise a cationic detergent. In one embodiment a surfactant containing substance may comprise a zwitterionic detergent.

In one embodiment a surfactant containing substance may comprise one or more PIF as described herein.

In one embodiment, one or more surfactant containing substance may be at least part of a polymer comprised of at least one or more surfactant containing substances.

In one embodiment one or more composition comprising one or more RNA substance and one or more surfactant containing substance may comprise one or more water concentrations in the water concentration list or in the water concentration range list. In one embodiment one or more composition comprising one or more RNA substance, one or more surfactant containing substance, and one or more additional substance, such as one or more cellular uptake agent or one or more additional RNA stabilizing substance as non-limiting examples, may comprise one or more water concentrations in the water concentration list or in the water concentration range list. In one embodiment one or more composition comprising one or more RNA substance and one or more surfactant containing substance may have a dielectric constant of one or more dielectric constant in the dielectric constant list or in the dielectric constant range list. In one embodiment one or more composition comprising one or more RNA substance, one or more surfactant containing substance, and one or more additional substance, such as one or more cellular uptake agent or one or more additional RNA stabilizing substance as non-limiting examples, may have a dielectric constant of one or more dielectric constant in the dielectric constant list or in the dielectric constant range list.

Embodiments of the present invention may include compositions comprising one or more surfactant containing substance used in a composition comprising one or more RNA substance, one or more surfactant containing substance, and one or more of the following substances: water, cellular uptake agents, aprotic substances, low dielectric substances, cyclic phosphate or metaphosphate containing substances, non-carbohydrate organic osmolyte substances, tertiary sulfonium containing substances, quaternary ammonium containing substances, quaternary phosphonium containing substances, hydrotrope containing substances, betaine containing substances, stabilizing monomers, stabilizing polymers, or supplemental RNA stabilizing substances. Other embodiments may include one or more composition comprising one or more surfactant containing substance and one or more RNA substance and may also comprise one or more cellular uptake agent or one or more additional RNA stabilizing substances. Other embodiments may include compositions comprising one or more surfactant containing substance, one or more RNA substance, and one or more additional substances, such as: one or more cellular uptake agent, or one or more additional RNA stabilizing substance, or one or more buffering agent, or one or more chelating agent, or one or more inorganic salt, or water, as non-limiting examples.

Embodiments of the present invention may include one or more compositions comprising one or more RNA substance and one or more surfactant containing substance, wherein a composition is not lyophilized.

In one embodiment one or more composition comprising one or more RNA substance and one or more surfactant containing substance may not be lyophilized. In one embodiment one or more composition comprising one or more RNA substance, one or more surfactant containing substance, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more surfactant containing substance, wherein a composition may be lyophilized.

In one embodiment one or more composition comprising one or more RNA substance and one or more surfactant containing substance may be lyophilized. In one embodiment one or more composition comprising one or more RNA substance, one or more surfactant containing substance, and one or more additional substance, such as one or more cellular uptake agent or one or more additional RNA stabilizing substance as non-limiting examples, may be lyophilized.

Embodiments of the present invention may include one or more compositions comprising one or more surfactant containing substance and one or more RNA substance described herein, wherein one or more composition described herein may have a melting of one or more melting point in the stabilizing composition melting point list.

In one embodiment one or more composition comprising one or more RNA substance and one or more surfactant containing substance may have a melting point of one or more melting point in the stabilizing composition melting point list. In one embodiment one or more composition comprising one or more RNA substance, one or more surfactant containing substance, and one or more additional substance, such as one or more cellular uptake agent or one or more additional RNA stabilizing substance as a non-limiting example, may have a melting point of one or more melting point in the stabilizing composition melting point list.

### Betaine Containing Substances:

The inventors have discovered that betaine containing substances may stabilize RNA substances.

The inventors have discovered that RNA stabilizing substances may comprise betaine containing substances. The inventors have also discovered that the stability of RNA substances may be enhanced in an RNA storage environment comprising a betaine containing substance. The inventors have also discovered that the stability of RNA substances may be enhanced in an RNA storage environment comprising a betaine containing substance and one or more additional RNA stabilizing substance, such as an aprotic substance.

The inventors have also discovered that the stability of RNA substances may be enhanced in compositions comprising an RNA substance and a betaine containing substance. The inventors have also discovered that the stability of RNA substances may be enhanced in compositions comprising an RNA substance, a betaine containing substance, and one or more additional RNA stabilizing substance, such as an aprotic substance.

In one embodiment of the present invention, one or more RNA stabilizing substance may comprise a betaine containing substance.

One embodiment of the present invention may include a composition comprised of one or more RNA substance and one or more betaine containing substance. Another embodiment of the present invention may include a composition comprised of one or more RNA substance, one or more betaine containing substance, and one or more additional RNA stabilizing substance, such as an aprotic substance.

One embodiment of the present invention may include a combination or mixture comprising one or more RNA substance and one or more betaine containing substance. Another embodiment of the present invention may include a combination or mixture comprising one or more RNA substance, one or more betaine containing substance, and one or more additional RNA stabilizing substance, such as an aprotic substance.

Embodiments of the present invention that comprise one or more RNA substance and one or more betaine containing substance may include combining, such as by mixing, one or more RNA substance with one or more substance that comprises at least one or more betaine containing substance.

Embodiments of the present invention that comprise one or more RNA substance, one or more betaine containing substance, and one or more additional RNA stabilizing substance, such as an aprotic substance, may include combining, such as by mixing, one or more RNA substance with one or more substance that comprises at least one or more betaine containing substance and one or more substance that comprises at least one or more RNA stabilizing substance, such as an aprotic substance.

An embodiment of the present invention may include compositions of materials that comprise one or more RNA substance and at least one or more betaine containing substance. The storage environment that improves the stability of RNA substances may comprise at least one or more vapor, liquid, powder, or solid betaine containing substance.

An embodiment of the present invention may include compositions of materials that comprise one or more RNA substance, one or more betaine containing substance, and one or more additional RNA stabilizing substance, such as an aprotic substance. The storage environment that improves the stability of RNA substances may comprise at least one or more vapor, liquid, powder, or solid betaine containing substance and may comprise at least one or more vapor, liquid, powder, or solid RNA stabilizing substance, such as an aprotic substance.

An embodiment of the present invention may include combinations of materials that comprise one or more RNA substance and at least one or more betaine containing substance. The storage environment that improves the stability of RNA substances may comprise at least one or more vapor, liquid, powder, or solid betaine containing substance.

An embodiment of the present invention may include combinations of materials that comprise one or more RNA substance, one or more betaine containing substance, and one or more additional RNA stabilizing substance, such as an aprotic substance. The storage environment that improves the stability of RNA substances may comprise at least one or more vapor, liquid, powder, or solid betaine containing substance and may comprise at least one or more vapor, liquid, powder, or solid RNA stabilizing substance, such as an aprotic substance.

In one embodiment of the present invention a composition comprised of one or more RNA substance and one or more betaine containing substance, produces a mixture with at least one or more RNA substance and at least one or more betaine containing substance. In one embodiment of the present invention a composition comprised of one or more RNA substance, one or more betaine containing substance, and one or more additional RNA stabilizing substance, such as an aprotic substance, produces a mixture with at least one or more RNA substance, at least one or more betaine containing substance, and at least one or more additional RNA stabilizing substance, such as an aprotic substance.

In one embodiment of the present invention a combination comprising one or more RNA substance and one or more betaine containing substance, produces a mixture with at least one or more RNA substance and at least one or more betaine containing substance. In one embodiment of the present invention a combination comprising one or more RNA substance, one or more betaine containing substance, and one or more additional RNA stabilizing substance, such as an aprotic substance, produces a mixture with at least one or more RNA substance, at least one or more betaine containing substance, and at least one or more additional RNA stabilizing substance, such as an aprotic substance.

In one embodiment of the present invention a composition with improved RNA stability may comprise one or more RNA substance and one or more betaine containing substance. In one embodiment of the present invention a composition with improved RNA stability may comprise one or more RNA substance, one or more betaine containing substance, and one or more additional RNA stabilizing substance, such as an aprotic substance.

In one embodiment of the present invention a composition with improved RNA stability may comprise a mixture of one or more RNA substance and one or more betaine containing substance. In one embodiment of the present invention a composition with improved RNA stability may comprise a mixture of one or more RNA substance, one or more betaine containing substance, and one or more additional RNA stabilizing substance, such as an aprotic substance.

In one embodiment of the present invention, each component included in a composition comprising one or more RNA substance and one or more betaine containing substance, may be stored separately, such as in a kit, or such as individual substances or as mixtures of one or more substance, and then combined later to produce a composition comprising one or more RNA substance and one or more betaine containing substance.

In one embodiment of the present invention, each component included in a composition comprising one or more RNA substance, one or more betaine containing substance, and one or more additional RNA stabilizing substance, such as an aprotic substance, may be stored separately, such as in a kit, or such as individually or as mixtures of one or more substance, and then combined later to produce a composition comprising one or more RNA substance, one or more betaine containing substance, and one or more additional RNA stabilizing substance, such as an aprotic substance.

In one embodiment of the present invention a composition comprising one or more RNA substance and one or more betaine containing substance may be at least partially biocompatible.

In one embodiment of the present invention a composition comprising one or more RNA substance, one or more betaine containing substance, and one or more additional RNA stabilizing substance, such as an aprotic substance, may be at least partially biocompatible.

In one embodiment of the present invention a combination comprised of one or more RNA substance and one or more betaine containing substance may be at least partially biocompatible.

In one embodiment of the present invention a combination comprised of one or more RNA substance, one or more betaine containing substance, and one or more additional RNA stabilizing substance, such as an aprotic substance, may be at least partially biocompatible.

Embodiments of the present invention comprising at least one or more betaine containing substances may include one or more forms of the substance. These forms may include, but are not limited to, one more counterions, ionic forms, conjugate bases, conjugate acids, protonated or deprotonated forms, hydrated or dehydrated forms, isomers, structural isomers, stereo isomers, chiral forms, salts, or combinations thereof.

In one embodiment a betaine containing substance may comprise one or more cation or cationic moiety. In one embodiment a betaine containing substance may comprise 1 or more cations or cationic moieties, 2 or more cations or cationic moieties, 3 or more cations or cationic moieties, 4 or more cations or cationic moieties, 5 or more cations or cationic moieties, 10 or more cations or cationic moieties, or 50 or more cations or cationic moieties. In one embodiment a betaine containing substance may comprise between about 1-100 cations or cationic moieties, or between about 1-50 cations or cationic moieties, or between about 1-20 cations or cationic moieties, or between about 1-10 cations or cationic moieties, or between about 1-5 cations or cationic moieties, or between about 1-3 cations or cationic moieties, or between about 1-2 cations or cationic moieties, or between about 2-100 cations or cationic moieties, or between about 2-50 cations or cationic moieties, or between about 2-20 cations or cationic moieties, or between about 2-10 cations or cationic moieties, or between about 2-5 cations or cationic moieties, or between about 2-3 cations or cationic moieties.

In one embodiment one or more cationic moiety of a betaine containing substance may comprise a nitrogen atom. In one embodiment one or more cationic moiety of a betaine containing substance may comprise a sulfur atom.

In one embodiment a betaine containing substance may comprise one or more quaternary ammonium. In one embodiment a betaine containing substance may comprise one or more tertiary sulfonium.

In one embodiment one or more cation or cationic moiety of a betaine containing substance may comprise a nitrogen atom. In one embodiment one or more cation or cationic moiety of a betaine containing substance may comprise a sulfur atom.

In one embodiment one or more cation or cationic moiety of a betaine containing substance may comprise a quaternary ammonium. In one embodiment one or more cation or cationic moiety of a betaine containing substance may comprise a tertiary sulfonium.

In one embodiment a betaine containing substance may comprise one or more quaternary ammonium cation or cationic moiety. In one embodiment a betaine containing substance may comprise one or more tertiary sulfonium cation or cationic moiety.

In one embodiment a betaine containing substance may comprise one or more anion or anionic moiety. In one embodiment a betaine containing substance may comprise 1 or more anions or anionic moieties, 2 or more anions or anionic moieties, 3 or more anions or anionic moieties, 4 or more anions or anionic moieties, 5 or more anions or anionic moieties, 10 or more anions or anionic moieties, or 50 or more anions or anionic moieties. In one embodiment a betaine containing substance may comprise between about 1-100 anions or anionic moieties, or between about 1-50 anions or anionic moieties, or between about 1-20 anions or anionic moieties, or between about 1-10 anions or anionic moieties, or between about 1-5 anions or anionic moieties, or between about 1-3 anions or anionic moieties, or between about 1-2 anions or anionic moieties, or between about 2-100 anions or anionic moieties, or between about 2-50 anions or anionic moieties, or between about 2-20 anions or anionic moieties, or between about 2-10 anions or anionic moieties, or between about 2-5 anions or anionic moieties, or between about 2-3 anions or anionic moieties.

In one embodiment a betaine containing substance may comprise one or more carboxylate group. In one embodiment a betaine containing substance may comprise one or more sulfonate group. In one embodiment a betaine containing substance may comprise one or more organophosphate group. In one embodiment a betaine containing substance may comprise one or more organosulfate group.

In one embodiment one or more anion or anionic moiety of a betaine containing substance may comprise a carboxylate group. In one embodiment one or more anion or anionic moiety of a betaine containing substance may comprise a sulfonate group. In one embodiment one or more anion or anionic moiety of a betaine containing substance may comprise an organophosphate group. In one embodiment one or more anion or anionic moiety of a betaine containing substance may comprise an organo sulfate group.

In one embodiment a betaine containing substance may comprise one or more carboxylate anion or anionic moiety. In one embodiment a betaine containing substance may comprise one or more sulfonate anion or anionic moiety. In one embodiment a betaine containing substance may comprise one or more organophosphate anion or anionic moiety. In one embodiment a betaine containing substance may comprise one or more organosulfate anion or anionic moiety.

In one embodiment a betaine containing substance may be hydroxylated. In one embodiment a betaine containing substance may comprise one or more hydroxy groups. In one embodiment a betaine containing substance may comprise 1 or more hydroxy groups, 2 or more hydroxy groups, 3 or more hydroxy groups, 4 or more hydroxy groups, 5 or more hydroxy groups, or more hydroxy groups, or 50 or more hydroxy groups. In one embodiment a betaine containing substance may comprise between about 1-100 hydroxy groups, or between about 1-50 hydroxy groups, or between about 1-20 hydroxy groups, or between about 1-10 hydroxy groups, or between about 1-5 hydroxy groups, or between about 1-3 hydroxy groups, or between about 2-100 hydroxy groups, or between about 2-50 hydroxy groups, or between about 2-20 hydroxy groups, or between about 2-10 hydroxy groups, or between about 2-5 hydroxy groups, or between about 2-3 hydroxy groups.

In one embodiment a betaine containing substance may be carboxylated. In one embodiment a betaine containing substance may comprise one or more carboxylate or carboxylic acid groups. In one embodiment a betaine containing substance may comprise 1 or more carboxylate or carboxylic acid groups, 2 or more carboxylate or carboxylic acid groups, 3 or more carboxylate or carboxylic acid groups, 4 or more carboxylate or carboxylic acid groups, 5 or more carboxylate or carboxylic acid groups, 10 or more carboxylate or carboxylic acid groups, or 50 or more carboxylate or carboxylic acid groups. In one embodiment a betaine containing substance may comprise between about 1-100 carboxylate or carboxylic acid groups, or between about 1-50 carboxylate or carboxylic acid groups, or between about 1-20 carboxylate or carboxylic acid groups, or between about 1-10 carboxylate or carboxylic acid groups, or between about 1-5 carboxylate or carboxylic acid groups, or between about 1-3 carboxylate or carboxylic acid groups, or between about 2-100 carboxylate or carboxylic acid groups, or between about 2-50 carboxylate or carboxylic acid groups, or between about 2-20 carboxylate or carboxylic acid groups, or between about 2-10 carboxylate or carboxylic acid groups, or between about 2-5 carboxylate or carboxylic acid groups, or between about 2-3 carboxylate or carboxylic acid groups. In one embodiment a betaine containing substance may comprise one or more ester bond.

In one embodiment a betaine containing substance may comprise 1 or more ester bonds, 2 or more ester bonds, 3 or more ester bonds, 4 or more ester bonds, 5 or more ester bonds, 10 or more ester bonds, or 50 or more ester bonds. In one embodiment a betaine containing substance may comprise between about 1-100 ester bonds, or between about 1-50 ester bonds, or between about 1-20 ester bonds, or between about 1-10 ester bonds, or between about 1-5 ester bonds, or between about 1-3 ester bonds, or between about 2-100 ester bonds, or between about 2-50 ester bonds, or between about 2-20 ester bonds, or between about 2-10 ester bonds, or between about 2-5 ester bonds, or between about 2-3 ester bonds.

In one embodiment a betaine containing substance may comprise one or more ester, such as a carboxylate ester or carbonate ester as non-limiting examples. In one embodiment a betaine containing substance may comprise 1 or more esters, 2 or more esters, 3 or more esters, 4 or more esters, 5 or more esters, 10 or more esters, or 50 or more esters. In one embodiment a betaine containing substance may comprise between about 1-100 esters, or between about 1-50 esters, or between about 1-20 esters, or between about 1-10 esters, or between about 1-5 esters, or between about 1-3 esters, or between about 2-100 esters, or between about 2-50 esters, or between about 2-esters, or between about 2-10 esters, or between about 2-5 esters, or between about 2-3 esters.

In one embodiment a betaine containing substance may comprise one or more ether bond. In one embodiment a betaine containing substance may comprise 1 or more ether bonds, 2 or more ether bonds, 3 or more ether bonds, 4 or more ether bonds, 5 or more ether bonds, 10 or more ether bonds, or 50 or more ether bonds. In one embodiment a betaine containing substance may comprise between about 1-100 ether bonds, or between about 1-50 ether bonds, or between about 1-20 ether bonds, or between about 1-10 ether bonds, or between about 1-5 ether bonds, or between about 1-3 ether bonds, or between about 2-100 ether bonds, or between about 2-50 ether bonds, or between about 2-20 ether bonds, or between about 2-10 ether bonds, or between about 2-5 ether bonds, or between about 2-3 ether bonds.

In one embodiment a betaine containing substance may comprise one or more ether. In one embodiment a betaine containing substance may comprise 1 or more ethers, 2 or more ethers, 3 or more ethers, 4 or more ethers, 5 or more ethers, 10 or more ethers, or 50 or more ethers. In one embodiment a betaine containing substance may comprise between about 1-100 ethers, or between about 1-50 ethers, or between about 1-20 ethers, or between about 1-10 ethers, or between about 1-5 ethers, or between about 1-3 ethers, or between about 2-100 ethers, or between about 2-50 ethers, or between about 2-20 ethers, or between about 2-10 ethers, or between about 2-5 ethers, or between about 2-3 ethers.

In one embodiment a betaine containing substance may comprise one or more amide bond. In one embodiment a betaine containing substance may comprise 1 or more amide bonds, 2 or more amide bonds, 3 or more amide bonds, 4 or more amide bonds, 5 or more amide bonds, 10 or more amide bonds, or 50 or more amide bonds. In one embodiment a betaine containing substance may comprise between about 1-100 amide bonds, or between about 1-50 amide bonds, or between about 1-20 amide bonds, or between about 1-10 amide bonds, or between about 1-5 amide bonds, or between about 1-3 amide bonds, or between about 2-100 amide bonds, or between about 2-50 amide bonds, or between about 2-20 amide bonds, or between about 2-10 amide bonds, or between about 2-5 amide bonds, or between about 2-3 amide bonds.

In one embodiment a betaine containing substance may comprise one or more amide group. In one embodiment a betaine containing substance may comprise 1 or more amide groups, 2 or more amide groups, 3 or more amide groups, 4 or more amide groups, 5 or more amide groups, 10 or more amide groups, or 50 or more amide groups. In one embodiment a betaine containing substance may comprise between about 1-100 amide groups, or between about 1-50 amide groups, or between about 1-20 amide groups, or between about 1-10 amide groups, or between about 1-5 amide groups, or between about 1-3 amide groups, or between about 2-100 amide groups, or between about 2-50 amide groups, or between about 2-20 amide groups, or between about 2-10 amide groups, or between about 2-5 amide groups, or between about 2-3 amide groups.

In one embodiment a betaine containing substance may comprise one or more organosulfate group. In one embodiment a betaine containing substance may comprise 1 or more organosulfate groups, 2 or more organosulfate groups, 3 or more organosulfate groups, 4 or more organosulfate groups, 5 or more organosulfate groups, 10 or more organosulfate groups, or 50 or more organosulfate groups. In one embodiment a betaine containing substance may comprise between about 1-100 organosulfate groups, or between about 1-50 organosulfate groups, or between about 1-20 organosulfate groups, or between about 1-10 organosulfate groups, or between about 1-5 organosulfate groups, or between about 1-3 organosulfate groups, or between about 2-100 organosulfate groups, or between about 2-50 organosulfate groups, or between about 2-20 organosulfate groups, or between about 2-10 organosulfate groups, or between about 2-5 organosulfate groups, or between about 2-3 organosulfate groups.

In one embodiment a betaine containing substance may comprise one or more sulfonate group. In one embodiment a betaine containing substance may comprise 1 or more sulfonate groups, 2 or more sulfonate groups, 3 or more sulfonate groups, 4 or more sulfonate groups, 5 or more sulfonate groups, 10 or more sulfonate groups, or 50 or more sulfonate groups. In one embodiment a betaine containing substance may comprise between about 1-100 sulfonate groups, or between about 1-50 sulfonate groups, or between about 1-20 sulfonate groups, or between about 1-10 sulfonate groups, or between about 1-5 sulfonate groups, or between about 1-3 sulfonate groups, or between about 2-100 sulfonate groups, or between about 2-50 sulfonate groups, or between about 2-20 sulfonate groups, or between about 2-10 sulfonate groups, or between about 2-5 sulfonate groups, or between about 2-3 sulfonate groups.

In one embodiment a betaine containing substance may comprise one or more organophosphate group. In one embodiment a betaine containing substance may comprise 1 or more organophosphate groups, 2 or more organophosphate groups, 3 or more organophosphate groups, 4 or more organophosphate groups, 5 or more organophosphate groups, 10 or more organophosphate groups, or 50 or more organophosphate groups. In one embodiment a betaine containing substance may comprise between about 1-100 organophosphate groups, or between about 1-50 organophosphate groups, or between about 1-20 organophosphate groups, or between about 1-10 organophosphate groups, or between about 1-5 organophosphate groups, or between about 1-3 organophosphate groups, or between about 2-100 organophosphate groups, or between about 2-50 organophosphate groups, or between about 2-20 organophosphate groups, or between about 2-10 organophosphate groups, or between about 2-5 organophosphate groups, or between about 2-3 organophosphate groups.

In one embodiment a betaine containing substance may not comprise a hydroxy group. In one embodiment a betaine containing substance may not comprise a carboxylate or carboxylic acid group. In one embodiment a betaine containing substance may not comprise an ester bond. In one embodiment a betaine containing substance may not comprise an ester. In one embodiment a betaine containing substance may not comprise an ether. In one embodiment a betaine containing substance may not comprise an ether bond. In one embodiment a betaine containing substance may not comprise an organosulfate group. In one embodiment a betaine containing substance may not comprise a sulfonate group. In one embodiment a betaine containing substance may not comprise an organophosphate group.

In one embodiment a betaine containing substance may comprise one or more polymer. In one embodiment a betaine containing substance may be polymeric. In one embodiment a betaine containing substance may comprise one or more cation. In one embodiment a betaine containing substance may comprise one or more anion. In one embodiment a betaine containing substance may comprise one or more zwitterion. In one embodiment a betaine containing substance may be zwitterionic.

In one embodiment a betaine containing substance may comprise one or more ring structure. In one embodiment a betaine containing substance may comprise one or more heterocyclic ring structure. In one embodiment a betaine containing substance may comprise one or more five membered ring structure. In one embodiment a betaine containing substance may comprise one or more six membered ring structure. In one embodiment a betaine containing substance may comprise one or more heterocyclic six membered ring structure. In one embodiment a betaine containing substance may comprise one or more heterocyclic five membered ring structure. In one embodiment a betaine containing substance may comprise one or more nitrogen heterocycles.

In one embodiment a betaine containing substance may comprise one or more cation that may be at least part of one or more nitrogen heterocycle, such as a five membered or six membered nitrogen heterocycle as non-limiting examples. In one embodiment a betaine containing substance may comprise 1 or more cations that may be at least part of a five membered or six membered nitrogen heterocycle, 2 or more cations that may be at least part of a five membered or six membered nitrogen heterocycle, 3 or more cations that may be at least part of a five membered or six membered nitrogen heterocycle, 4 or more cations that may be at least part of a five membered or six membered nitrogen heterocycle, 5 or more cations that may be at least part of a five membered or six membered nitrogen heterocycle, 10 or more cations that may be at least part of a five membered or six membered nitrogen heterocycle, or 50 or more cations that may be at least part of a five membered or six membered nitrogen heterocycle. In one embodiment a betaine containing substance may comprise between about 1-100 cations that may be at least part of a five membered or six membered nitrogen heterocycle, or between about 1-50 cations that may be at least part of a five membered or six membered nitrogen heterocycle, or between about 1-20 cations that may be at least part of a five membered or six membered nitrogen heterocycle, or between about 1-10 cations that may be at least part of a five membered or six membered nitrogen heterocycle, or between about 1-5 cations that may be at least part of a five membered or six membered nitrogen heterocycle, or between about 1-3 cations that may be at least part of a five membered or six membered nitrogen heterocycle, or between about 1-2 cations that may be at least part of a five membered or six membered nitrogen heterocycle, or between about 2-100 cations that may be at least part of a five membered or six membered nitrogen heterocycle, or between about 2-50 cations that may be at least part of a five membered or six membered nitrogen heterocycle, or between about 2-20 cations that may be at least part of a five membered or six membered nitrogen heterocycle, or between about 2-10 cations that may be at least part of a five membered or six membered nitrogen heterocycle, or between about 2-5 cations that may be at least part of a five membered or six membered nitrogen heterocycle, or between about 2-3 cations that may be at least part of a five membered or six membered nitrogen heterocycle.

In one embodiment a betaine containing substance may comprise one or more pyridinium group. In one embodiment a betaine containing substance may comprise one or more pyrrolidinium group. In one embodiment a betaine containing substance may comprise one or more imidazolium group. In one embodiment a betaine containing substance may comprise one or more piperidinium group. In one embodiment a betaine containing substance may comprise one or more indolium group. In one embodiment a betaine containing substance may comprise one or more pyrimidinium group. In one embodiment a betaine containing substance may comprise one or more purinium group.

In one embodiment a betaine containing substance may comprise one or more cation that may be at least part of one or more nitrogen heterocycle or aromatic nitrogen heterocycle, such as a pyridinium, pyrrolidinium, imidazolium, piperidinium, indolium, pyrimidinium, or purinium group as non-limiting examples.

In one embodiment a betaine containing substance may comprise 1 or more pyridinium, pyrrolidinium, imidazolium, piperidinium, indolium, pyrimidinium, or purinium groups, 2 or more pyridinium, pyrrolidinium, imidazolium, piperidinium, indolium, pyrimidinium, or purinium groups, 3 or more pyridinium, pyrrolidinium, imidazolium, piperidinium, indolium, pyrimidinium, or purinium groups, 4 or more pyridinium, pyrrolidinium, imidazolium, piperidinium, indolium, pyrimidinium, or purinium groups, 5 or more pyridinium, pyrrolidinium, imidazolium, piperidinium, indolium, pyrimidinium, or purinium groups, 10 or more pyridinium, pyrrolidinium, imidazolium, piperidinium, indolium, pyrimidinium, or purinium groups, or 50 or more pyridinium, pyrrolidinium, imidazolium, piperidinium, indolium, pyrimidinium, or purinium groups. In one embodiment a betaine containing substance may comprise between about 1-100 pyridinium, pyrrolidinium, imidazolium, piperidinium, indolium, pyrimidinium, or purinium groups, or between about 1-50 pyridinium, pyrrolidinium, imidazolium, piperidinium, indolium, pyrimidinium, or purinium groups, or between about 1-20 pyridinium, pyrrolidinium, imidazolium, piperidinium, indolium, pyrimidinium, or purinium groups, or between about 1-10 pyridinium, pyrrolidinium, imidazolium, piperidinium, indolium, pyrimidinium, or purinium groups, or between about 1-5 pyridinium, pyrrolidinium, imidazolium, piperidinium, indolium, pyrimidinium, or purinium groups, or between about 1-3 pyridinium, pyrrolidinium, imidazolium, piperidinium, indolium, pyrimidinium, or purinium groups, or between about 1-2 pyridinium, pyrrolidinium, imidazolium, piperidinium, indolium, pyrimidinium, or purinium groups, or between about 2-100 pyridinium, pyrrolidinium, imidazolium, piperidinium, indolium, pyrimidinium, or purinium groups, or between about 2-50 pyridinium, pyrrolidinium, imidazolium, piperidinium, indolium, pyrimidinium, or purinium groups, or between about 2-20 pyridinium, pyrrolidinium, imidazolium, piperidinium, indolium, pyrimidinium, or purinium groups, or between about 2-10 pyridinium, pyrrolidinium, imidazolium, piperidinium, indolium, pyrimidinium, or purinium groups, or between about 2-5 pyridinium, pyrrolidinium, imidazolium, piperidinium, indolium, pyrimidinium, or purinium groups, or between about 2-3 pyridinium, pyrrolidinium, imidazolium, piperidinium, indolium, pyrimidinium, or purinium groups.

In one embodiment a betaine containing substance may comprise one or more aliphatic quaternary ammonium. In one embodiment a betaine containing substance may comprise one or more quaternary ammonium nitrogen heterocycle. In one embodiment a betaine containing substance may comprise one or more quaternary ammonium aromatic nitrogen heterocycle. In one embodiment a betaine containing substance may comprise one or more five membered quaternary ammonium nitrogen heterocycle. In one embodiment a betaine containing substance may comprise one or more six membered quaternary ammonium nitrogen heterocycle. In one embodiment a betaine containing substance may comprise one or more five membered aromatic quaternary ammonium nitrogen heterocycle. In one embodiment a betaine containing substance may comprise one or more six membered aromatic quaternary ammonium nitrogen heterocycle.

In one embodiment a betaine containing substance may comprise one or more cation or cationic moiety. In one embodiment a betaine containing substance may comprise one or more anion or anionic moiety. In one embodiment a betaine containing substance may comprise one or more zwitterion or zwitterionic moiety. In one embodiment a betaine containing substance may be zwitterionic.

In one embodiment a betaine containing substance may comprise one or more carboxybetaine. In one embodiment a betaine containing substance may comprise one or more sulfobetaine, such as a non-detergent sulfobetaine as a non-limiting example.

In one embodiment a betaine containing substance may comprise one or more quaternary ammonium cation that may be at least part of one or more nitrogen heterocycle or aromatic nitrogen heterocycle, such as a pyridinium, pyrrolidinium, imidazolium, piperidinium, indolium, pyrimidinium, or purinium group as non-limiting examples.

In one embodiment a betaine containing substance may comprise one or more betaines. In one embodiment a betaine containing substance may comprise 1 or more betaines, 2 or more betaines, 3 or more betaines, 4 or more betaines, 5 or more betaines, 10 or more betaines, 50 or more betaines, or 100 or more betaines. In one embodiment a betaine containing substance may comprise between about 1-1,000 betaines, or between about 1-500 betaines, or between about 1-200 betaines, or between about 1-100 betaines, or between about 1-50 betaines, or between about 1-20 betaines, or between about 1-10 betaines, or between about 1-5 betaines, or between about 1-3 betaines, or between about 2-1,000 betaines, or between about 2-500 betaines, or between about 2-200 betaines, or between about 2-100 betaines, or between about 2-50 betaines, or between about 2-20 betaines, or between about 2-10 betaines, or between about 2-5 betaines, or between about 2-3 betaines.

In one embodiment a betaine containing substance may comprise one or more cationic moiety that is at least part of a heterocyclic ring structure. In one embodiment a betaine containing substance may comprise one or more cationic moiety that is at least part of a five membered heterocyclic ring structure. In one embodiment a betaine containing substance may comprise one or more cationic moiety that is at least part of a six membered heterocyclic ring structure. In one embodiment a betaine containing substance may comprise one or more cationic moiety that is at least part of an aromatic six membered heterocyclic ring structure.

In one embodiment a betaine containing substance may be monomeric. In one embodiment a betaine containing substance may not be polymeric.

In one embodiment a betaine containing substance may comprise a ring structure. In one embodiment a betaine containing substance may comprise a heterocyclic ring structure. In one embodiment a betaine containing substance may comprise a five membered ring structure. In one embodiment a betaine containing substance may comprise a six membered ring structure. In one embodiment a betaine containing substance may comprise a heterocyclic six membered ring structure. In one embodiment a betaine containing substance may comprise a heterocyclic five membered ring structure. In one embodiment a betaine containing substance may comprise a nitrogen heterocycle. In one embodiment a betaine containing substance may comprise an aromatic nitrogen heterocycle.

In one embodiment a betaine containing substance may comprise a cationic moiety that is at least part of a heterocyclic ring structure. In one embodiment a betaine containing substance may comprise a cationic moiety that is at least part of a five membered heterocyclic ring structure. In one embodiment a betaine containing substance may comprise a cationic moiety that is at least part of a six membered heterocyclic ring structure.

A non-limiting example of a betaine containing substance comprised of one or more betaine that may be used is trimethylglycine (TMG) (also known as TMG, glycine betaine, or N,N,N-trimethylglycine), wherein TMG may be substituted for or used in combination with one or more betaine containing substances described herein. One embodiment of the present invention may be a combination of substances comprising TMG and another embodiment may also comprise at least one RNA substance. Other embodiments may be combinations of substances comprising TMG wherein the concentration of TMG may be one or more concentration in the stabilizing substance weight percent concentration list or in the stabilizing substance molar concentration range list or in the stabilizing substance molar concentration list.

A non-limiting example of a betaine containing substance comprised of one or more betaine that may be used is NDSB-195 (also known as dimethylethylammoniumpropane sulfonate or ethyl dimethyl ammonio propane sulfonate), wherein NDSB-195 may be substituted for or used in combination with one or more betaine containing substances described herein. One embodiment of the present invention may be a combination of substances comprising NDSB-195 and another embodiment may also comprise at least one RNA substance. Other embodiments may be combinations of substances comprising NDSB-195 wherein the concentration of NDSB-195 may be one or more concentration in the stabilizing substance weight percent concentration list or in the stabilizing substance molar concentration range list or in the stabilizing substance molar concentration list.

A non-limiting example of a betaine containing substance comprised of one or more betaine that may be used is NDSB-201 (also known as 3-(1-pyridinio)-1-propane sulfonate), wherein NDSB-201 may be substituted for or used in combination with one or more betaine containing substances described herein. One embodiment of the present invention may be a combination of substances comprising NDSB-201 and another embodiment may also comprise at least one RNA substance. Other embodiments may be combinations of substances comprising NDSB-201 wherein the concentration of NDSB-201 may be one or more concentration in the stabilizing substance weight percent concentration list or in the stabilizing substance molar concentration range list or in the stabilizing substance molar concentration list.

A non-limiting example of a betaine containing substance comprised of one or more betaine that may be used is NDSB-221 (also known as 3-(1-methylpiperidinium)-1-propane sulfonate), wherein NDSB-221 may be substituted for or used in combination with one or more betaine containing substances described herein. One embodiment of the present invention may be a combination of substances comprising NDSB-221 and another embodiment may also comprise at least one RNA substance. Other embodiments may be combinations of substances comprising NDSB-221 wherein the concentration of NDSB-221 may be one or more concentration in the stabilizing substance weight percent concentration list or in the stabilizing substance molar concentration range list or in the stabilizing substance molar concentration list.

A non-limiting example of a betaine containing substance comprised of one or more betaine that may be used is L-alpha glycerylphosphorylcholine (alpha-GPC) (also known as alpha-GPC, glycerophosphocholine, or choline alfoscerate), wherein alpha-GPC may be substituted for or used in combination with one or more betaine containing substances described herein. One embodiment of the present invention may be a combination of substances comprising alpha-GPC and another embodiment may also comprise at least one RNA substance. Other embodiments may be combinations of substances comprising alpha-GPC wherein the concentration of alpha-GPC may be one or more concentration in the stabilizing substance weight percent concentration list or in the stabilizing substance molar concentration range list or in the stabilizing substance molar concentration list.

A non-limiting example of a betaine containing substance comprised of one or more betaine that may be used is carnitine, wherein carnitine may be substituted for or used in combination with one or more betaine containing substances described herein. One embodiment of the present invention may be a combination of substances comprising carnitine and another embodiment may also comprise at least one RNA substance. Other embodiments may be combinations of substances comprising carnitine wherein the concentration of carnitine may be one or more concentration in the stabilizing substance weight percent concentration list or in the stabilizing substance molar concentration range list or in the stabilizing substance molar concentration list.

A non-limiting example of a betaine containing substance comprised of one or more betaine that may be used is stachydrine (also known as proline betaine), wherein stachydrine may be substituted for or used in combination with one or more betaine containing substances described herein. One embodiment of the present invention may be a combination of substances comprising stachydrine and another embodiment may also comprise at least one RNA substance. Other embodiments may be combinations of substances comprising stachydrine wherein the concentration of stachydrine may be one or more concentration in the stabilizing substance weight percent concentration list or in the stabilizing substance molar concentration range list or in the stabilizing substance molar concentration list.

A non-limiting example of a betaine containing substance comprised of one or more betaine that may be used is valine betaine, wherein valine betaine may be substituted for or used in combination with one or more betaine containing substances described herein. One embodiment of the present invention may be a combination of substances comprising valine betaine and another embodiment may also comprise at least one RNA substance. Other embodiments may be combinations of substances comprising valine betaine wherein the concentration of valine betaine may be one or more concentration in the stabilizing substance weight percent concentration list or in the stabilizing substance molar concentration range list or in the stabilizing substance molar concentration list.

A non-limiting example of a betaine containing substance comprised of one or more betaine that may be used is choline-O-sulfate (COS) (also known as choline sulfate), wherein COS may be substituted for or used in combination with one or more betaine containing substances described herein. One embodiment of the present invention may be a combination of substances comprising COS and another embodiment may also comprise at least one RNA substance. Other embodiments may be combinations of substances comprising COS wherein the concentration of COS may be one or more concentration in the stabilizing substance weight percent concentration list or in the stabilizing substance molar concentration range list or in the stabilizing substance molar concentration list.

For conciseness, the following list of betaine containing substances, is herein referred to as the betaine containing substance list wherein one or more of the following substances may be substituted for or used in combination with one or more betaine containing substance as described herein.

In other embodiments, other betaine containing substances that may be substituted for or used in combination with one or more betaine containing substance as described herein may comprise one or more of the following, including but not limited to: phosphocholine, phosphorylcholine, acetylcholine, carnitine, crotonobetaine, butyrobetaine, gamma-butyrobetaine, trimethylglycine, TMG, betaine, glycine betaine, alanine betaine, beta-alanine betaine, phosphobetaine, phosphorylbetaine, betaine phosphate, glycerophosphorylcholine, glycerophosphocholine, choline-O-sulfate, choline sulfate, beta-dimethylsulfonopropionate, dimethylsulfoniopropionate (DMSP), hydroxyproline betaine, proline betaine, dimethyl proline, stachydrine, valine betaine, glutamic acid betaine, trigonelline, glutamine betaine, trimethyllysine, L-alpha glycerylphosphorylcholine (alpha-GPC), 4-trimethyl-ammoniobutanoate, trimethyl-ammoniobutanoate, pipecolic acid betaine, phosphobetaine, sulfobetaines, non-detergent sulfobetaines, dimethylethylammoniumpropane sulfonate, dimethylethylammonium-1-propanesulfonate, NDSB-195, 3-(1-pyridinio)-1-propane sulfonate, NDSB-201, NDSB-209, dimethyl-2-hydroxyethylammoniumpropane sulfonate, (2-hydroxyethyl)dimethyl(3-sulfopropyl)ammonium, 3-[dimethyl-(2-hydroxyethyl)ammonio]-1-propanesulfonate, NDSB-211, 3-(1-methylpiperidinium)-1-propane sulfonate, NDSB-221, NDSB-223, NDSB-225, NDSB-249, dimethylbenzylammonium propane sulfonate, 3-(benzyldimethylammonio)propanesulfonate, NDSB-256, 3-(4-tert-butyl-1-pyridinio)-1-propanesulfonate, NDSB-256-4T, betaine aldehyde, imidazolium betaine, piperidinium betaine, pyridinium betaine, pyrrolidinium betaine, morpholinium betaine, 4-(trimethylammonio)butanoate, actinine, deoxycarnitine, 4-dimethylsulphonio-2-hydroxybutyrate, 4-dimethylsulfonio-2-hydroxybutyrate, ethylmethylsulfoniopropionate diethylsulfoniopropionate, isopropylmethylsulfoniopropionate, tetramethylenesulfoniopropionate, methylpropylsulfoniopropionate 3-dimethylsulfoniopropionate, 2-dimethylsulfonioacetate, 2-dimethylsulfonioethanol (dimethylthioethanol), dimethylsulfoniopropionate-amine, dimethylsulfoniopropionate-aldehyde, methyl-dimethylsulfoniopropionate, S-Adenosyl methionine, S-Methylmethionine, (2-carboxyethyl)(dimethyl)sulfonium, dimethyl(2-hydroxy-5-nitrobenzyl)sulfonium, dimethyl(phenacyl)sulfonium, (ethoxycarbonylmethyl)dimethylsulfonium, dibutylsulfoniopropionate, butylethylsulfoniopropionate, butylmethylsulfoniopropionate, butylpropylsulfoniopropionate, diproprylsulfoniopropionate, diisopropylsulfoniopropionate, isopropylproprylsulfoniopropionate, isopropylethylsulfoniopropionate, isopropylbutylsulfoniopropionate, diethylsulfonioacetate, dibutylsulfonioacetate, ethylmethylsulfonioacetate, butylmethylsulfonioacetate, butylethylsulfonioacetate, dipropylsulfonioacetate, propylethylsulfonioacetate, porpylmethylsulfonioacetate, propylbutylsulfonioacetate, ispropylmethylsulfonioacetate, isopropylethylsulfonioacetate, isopropylbutylsulfonioacetate, isopropylpropylsulfonioacetate, diisopropylsulfonioacetate, sulfocholines, phosphatidylsulfocholines, carboxythetins, sulfothetins, sulfonium sulfonates, meldonium, arsenobetaine, betonicine, gamma-butyrobetaine, ergothioneine, propionobetaine, homostachydrine, phenylalanine betaine, homarine, isotrigonelline, taurine, dimethylthetin, hercynine, trigonelline methylester, trigonelline glucosylester, cocamidopropyl hydroxysultaine, cocamidopropyl betaine, trimethyl lysine, myristyl betaine, lauryl betaine, mesoionic compounds, mesomeric betaines, heterocyclic mesomeric betaines, acyclic mesomeric betaines, dimethylseleniopropionate, dimethyltelluriopropionate, and 4-(3-butyl-1-imidazolio)-1-butanesulfonate, or derivatives, or combinations thereof.

Embodiments of the present invention may include combinations comprising one or more of the substances from the betaine containing substance list substituted for or used in combination with one or more betaine containing substance or one or more RNA stabilizing substance, such as an aprotic substance as a non-limiting example, and other embodiments may also comprise at least one RNA substance.

Other embodiments may include combinations of substances comprising one or more betaine containing substance selected from the betaine containing substance list wherein the concentration of one or more substances may be one or more concentration in the stabilizing substance weight percent concentration list or in the stabilizing substance molar concentration range list or in the stabilizing substance molar concentration list.

In one embodiment, a betaine containing substance may have a molecular weight between about 50-1,000,000 daltons. In one embodiment, a betaine containing substance may have a molecular weight between about 50-500,000 daltons. In one embodiment, a betaine containing substance may have a molecular weight between about 50-100,000 daltons. In one embodiment, a betaine containing substance may have a molecular weight between about 50-50,000 daltons. In one embodiment, a betaine containing substance may have a molecular weight between about 50-20,000 daltons. In one embodiment, a betaine containing substance may have a molecular weight between about 50-10,000 daltons. In one embodiment, a betaine containing substance may have a molecular weight between about 50-5,000 daltons. In one embodiment, a betaine containing substance may have a molecular weight between about 50-2,000 daltons. In one embodiment, a betaine containing substance may have a molecular weight between about 50-1,000 daltons. In one embodiment, a betaine containing substance may have a molecular weight between about 50-900 daltons. In one embodiment, a betaine containing substance may have a molecular weight between about 50-800 daltons. In one embodiment, a betaine containing substance may have a molecular weight between about 50-700 daltons. In one embodiment, a betaine containing substance may have a molecular weight between about 50-600 daltons. In one embodiment, a betaine containing substance may have a molecular weight between about 50-500 daltons. In one embodiment, a betaine containing substance may have a molecular weight between about 50-400 daltons. In one embodiment, a betaine containing substance may have a molecular weight between about 50-300 daltons. In one embodiment, a betaine containing substance may have a molecular weight between about 50-250 daltons.

In one embodiment, a betaine containing substance may have a molecular weight between about 100-1,000,000 daltons. In one embodiment, a betaine containing substance may have a molecular weight between about 100-500,000 daltons. In one embodiment, a betaine containing substance may have a molecular weight between about 100-100,000 daltons. In one embodiment, a betaine containing substance may have a molecular weight between about 100-50,000 daltons. In one embodiment, a betaine containing substance may have a molecular weight between about 100-20,000 daltons. In one embodiment, a betaine containing substance may have a molecular weight between about 100-10,000 daltons. In one embodiment, a betaine containing substance may have a molecular weight between about 100-5,000 daltons. In one embodiment, a betaine containing substance may have a molecular weight between about 100-2,000 daltons. In one embodiment, a betaine containing substance may have a molecular weight between about 100-1,000 daltons. In one embodiment, a betaine containing substance may have a molecular weight between about 100-900 daltons. In one embodiment, a betaine containing substance may have a molecular weight between about 100-800 daltons. In one embodiment, a betaine containing substance may have a molecular weight between about 100-700 daltons. In one embodiment, a betaine containing substance may have a molecular weight between about 100-600 daltons. In one embodiment, a betaine containing substance may have a molecular weight between about 100-500 daltons. In one embodiment, a betaine containing substance may have a molecular weight between about 100-400 daltons. In one embodiment, a betaine containing substance may have a molecular weight between about 100-300 daltons. In one embodiment, a betaine containing substance may have a molecular weight between about 100-250 daltons.

One embodiment of the present invention is the method whereby one or more betaine containing substance may be combined, such as by mixing, with at least one or more RNA substance to produce a mixture comprising at least one or more betaine containing substance and at least one or more RNA substance. As a non-limiting example one or more RNA substance may be mixed with one or more betaine containing substance.

Another embodiment of the present invention is the method whereby one or more betaine containing substance may be combined, such as by mixing, with at least one or more RNA substance to produce a mixture comprising at least one or more RNA substance and at least one or more betaine containing substance at one or more of the RNA stabilizing substance concentrations within the stabilizing substance molar concentration range list or in the stabilizing substance molar concentration list or the stabilizing substance weight percent concentration list as described herein. These same methods may be used to combine one or more other substances, including, but not limited to one or more cellular uptake agents or one or more additional RNA stabilizing substances, with one or more RNA substances and one or more betaine containing substances to produce a mixture comprising one or more RNA substance, one or more betaine containing substance, and one or more cellular uptake agent or one or more additional RNA stabilizing substance.

One embodiment of the present invention is the method whereby one or more betaine containing substance may be combined, such as by mixing, with at least one or more RNA substance to produce a composition comprising at least one or more RNA substance and at least one or more betaine containing substance. Another embodiment of the present invention is the method whereby one or more betaine containing substance may be combined, such as by mixing, with one or more RNA substance to produce a composition comprising at least one or more RNA substance and at least one or more betaine containing substance at one or more of the RNA stabilizing substance concentrations within the stabilizing substance molar concentration range list or in the stabilizing substance molar concentration list or stabilizing substance weight percent concentration list as described herein. These same methods may be used to combine one or more other substances, including, but not limited to one or more cellular uptake agents or one or more additional RNA stabilizing substance, with one or more RNA substance and one or more betaine containing substance to produce a composition comprising one or more RNA substance, one or more betaine containing substance, and one or more cellular uptake agent or one or more additional RNA stabilizing substance.

In one embodiment a betaine containing substance may be aliphatic. In one embodiment a betaine containing substance may monomeric. In one embodiment a betaine containing substance may comprise a ring structure. In another embodiment a betaine containing substance may comprise a heterocyclic ring structure. In one embodiment a betaine containing substance may comprise an aromatic ring structure. In another embodiment a betaine containing substance may comprise an aromatic heterocyclic ring structure.

In one embodiment a betaine containing substance may comprise a cationic moiety at about physiologic pH. In one embodiment a betaine containing substance may comprise an anionic moiety at about physiologic pH. In one embodiment a betaine containing substance may comprise a zwitterion at about physiologic pH.

In one embodiment a betaine containing substance may comprise one or more PIF as described herein.

In one embodiment a betaine containing substance may monomeric. In one embodiment a betaine containing substance may be polymeric. In one embodiment a betaine containing substance may comprise a polymer.

In one embodiment, one or more betaine containing substance may be at least part of a polymer comprised of at least one or more betaine containing substances.

In one embodiment one or more composition comprising one or more RNA substance and one or more betaine containing substance may comprise one or more water concentrations in the water concentration list or in the water concentration range list. In one embodiment one or more composition comprising one or more RNA substance, one or more betaine containing substance, and one or more additional substance, such as one or more cellular uptake agent or one or more additional RNA stabilizing substance as non-limiting examples, may comprise one or more water concentrations in the water concentration list or in the water concentration range list. In one embodiment one or more composition comprising one or more RNA substance and one or more betaine containing substance may have a dielectric constant of one or more dielectric constant in the dielectric constant list or in the dielectric constant range list. In one embodiment one or more composition comprising one or more RNA substance, one or more betaine containing substance, and one or more additional substance, such as one or more cellular uptake agent or one or more additional RNA stabilizing substance as non-limiting examples, may have a dielectric constant of one or more dielectric constant in the dielectric constant list or in the dielectric constant range list.

Embodiments of the present invention may include compositions comprising one or more betaine containing substance used in a composition comprising one or more RNA substance, one or more betaine containing substance, and one or more of the following substances: water, cellular uptake agents, aprotic substances, low dielectric substances, cyclic phosphate or metaphosphate containing substances, non-carbohydrate organic osmolyte substances, tertiary sulfonium containing substances, quaternary ammonium containing substances, quaternary phosphonium containing substances, hydrotrope containing substances, surfactant containing substances, stabilizing monomers, stabilizing polymers, or supplemental RNA stabilizing substances. Other embodiments may include one or more composition comprising one or more betaine containing substance and one or more RNA substance and may also comprise one or more cellular uptake agent or one or more additional RNA stabilizing substances. Other embodiments may include compositions comprising one or more betaine containing substance, one or more RNA substance, and one or more additional substances, such as: one or more cellular uptake agent, or one or more additional RNA stabilizing substance, or one or more buffering agent, or one or more chelating agent, or one or more inorganic salt, or water, as non-limiting examples.

Embodiments of the present invention may include one or more compositions comprising one or more RNA substance and one or more betaine containing substance, wherein a composition is not lyophilized.

In one embodiment one or more composition comprising one or more RNA substance and one or more betaine containing substance may not be lyophilized. In one embodiment one or more composition comprising one or more RNA substance, one or more betaine containing substance, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more betaine containing substance, wherein a composition may be lyophilized.

In one embodiment one or more composition comprising one or more RNA substance and one or more betaine containing substance may be lyophilized. In one embodiment one or more composition comprising one or more RNA substance, one or more betaine containing substance, and one or more additional substance, such as one or more cellular uptake agent or one or more additional RNA stabilizing substance as non-limiting examples, may be lyophilized.

Embodiments of the present invention may include one or more compositions comprising one or more betaine containing substance and one or more RNA substance described herein, wherein one or more composition described herein may have a melting of one or more melting point in the stabilizing composition melting point list.

In one embodiment one or more composition comprising one or more RNA substance and one or more betaine containing substance may have a melting point of one or more melting point in the stabilizing composition melting point list. In one embodiment one or more composition comprising one or more RNA substance, one or more betaine containing substance, and one or more additional substance, such as one or more cellular uptake agent or one or more additional RNA stabilizing substance as a non-limiting example, may have a melting point of one or more melting point in the stabilizing composition melting point list.

### Stabilizing Monomers and Polymers:

The inventors have discovered that RNA stabilizing substances may comprise stabilizing polymers comprised of one or more stabilizing monomers.

The inventors have also discovered that stabilized RNA compositions may be comprised of one or more stabilizing monomers. Stabilizing monomers may be comprised of one or more NPS stabilizing moieties or one or more COPS stabilizing moieties. Stabilizing polymers, stabilizing monomers, NPS stabilizing moieties, and COPS stabilizing moieties are described herein.

### NPS Stabilizing Moiety:

The inventors have discovered that RNA stabilizing substances may comprise one or more quaternary ammonium, tertiary amine, quaternary phosphonium, or tertiary sulfonium, herein referred to as an NPS stabilizing moiety.

The inventors have also discovered that the stability of RNA substances may be enhanced in an RNA storage environment comprising an NPS stabilizing moiety, such as quaternary ammonium, tertiary amine, quaternary phosphonium, or tertiary sulfonium.

In one embodiment of the present invention an NPS stabilizing moiety may be comprised of one or more nitrogen atom, phosphorus atom, or sulfur atom. In another embodiment an NPS stabilizing moiety may be comprised of a nitrogen atom or a sulfur atom covalently bonded to at least three carbon atoms. In another embodiment an NPS stabilizing moiety may be comprised of a nitrogen atom or a phosphorus atom covalently bonded to at least four carbon atoms.

In a non-limiting example, an NPS stabilizing moiety may comprise a quaternary ammonium, a tertiary amine, a quaternary phosphonium, or a tertiary sulfonium, wherein the quaternary ammonium is comprised of a nitrogen atom covalently bonded at least 3 or more carbon atoms, the tertiary amine is comprised of a nitrogen atom covalently bonded to 3 carbon atoms, the quaternary phosphonium is comprised of a phosphorus atom covalently bonded to 4 carbon atoms, or the tertiary sulfonium is comprised of sulfur atom covalently bonded to 3 carbon atoms.

In one embodiment an NPS stabilizing moiety may be comprised of a nitrogen atom, a phosphorus atom, or a sulfur atom with at least three covalent bonds. In one embodiment an NPS stabilizing moiety may be comprised of a nitrogen atom or a phosphorus atom with more than three covalent bonds. In one embodiment an NPS stabilizing moiety may be comprised of a nitrogen atom or a phosphorus atom with at least four covalent bonds.

In one embodiment an NPS stabilizing moiety may be aprotic at about physiologic pH. In another embodiment an NPS stabilizing moiety may comprise an aprotic group at about physiologic pH.

In one embodiment an NPS stabilizing moiety may be cationic at about physiologic pH. In another embodiment an NPS stabilizing moiety may comprise one or more cationic group at about physiologic pH.

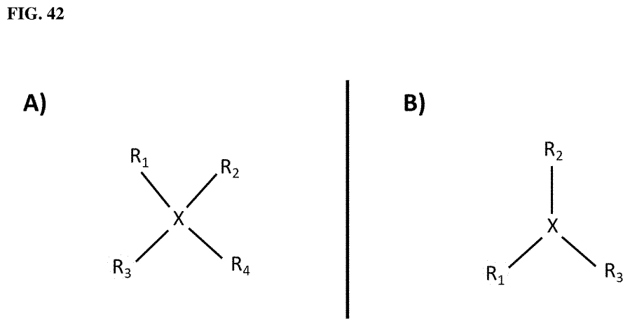

In one embodiment an NPS stabilizing moiety may be comprised of a substance according to FIG. 42A. Wherein X may be a nitrogen atom or phosphorus atom covalently bonded to 4 carbon atoms represented by R₁, R₂, R₃, and R₄. R₁, R₂, R₃, and R₄ may be independently selected R groups, as described herein, and R₁, R₂, R₃, and R₄ may be the same or different. In one embodiment, one or more of R₁, R₂, R₃, or R₄ may be interchangeable with one or more of the other R groups as described herein. As a non-limiting example, R₄ may be interchangeable with one or more other R group such as R₁, R₂, or R₃ as described herein, wherein R₄ may be replaced or exchange positions with one or more other R group such as R₁, R₂, or R₃.

In one embodiment an NPS stabilizing moiety may be comprised of a substance according to FIG. 42B. Wherein X may be a nitrogen atom or sulfur atom covalently bonded to 3 carbon atoms represented by R₁, R₂, and R₃. R₁, R₂, and R₃ may be independently selected R groups, as described herein, and R₁, R₂, and R₃ may be the same or different. In one embodiment, one or more of R₁, R₂, or R₃ may be interchangeable with one or more of the other R groups as described herein. As a non-limiting example, R₃ may be interchangeable with one or more other R group such as R₁ or R₂, as described herein, wherein R₃ may be replaced or exchange positions with one or more other R group such as R₁ or R₂.

In one embodiment an R group may be comprised of at least one carbon atom. Wherein, R may be comprised of a C1-35 alkyl, alkenyl, alkynyl, aryl, or aralkyl group, each of which may contain up to 20 heteroatoms. In another embodiment an R group may be comprised of C1-30 alkyl, alkenyl, alkynyl, aryl, or aralkyl group, each of which may contain up to 16 heteroatoms. In another embodiment an R group may be comprised of C1-24 alkyl, alkenyl, alkynyl, aryl, or aralkyl group, each of which may contain up to 12 heteroatoms. In another embodiment an R group may be comprised of C1-20 alkyl, alkenyl, alkynyl, aryl, or aralkyl group, each of which may contain up to 10 heteroatoms. In another embodiment an R group may be comprised of C1-16 alkyl, alkenyl, alkynyl, aryl, or aralkyl group, each of which may contain up to 8 heteroatoms. In another embodiment an R group may be comprised of C1-16 alkyl, alkenyl, alkynyl, aryl, or aralkyl group, each of which may contain up to 6 heteroatoms. In another embodiment an R group may be comprised of C1-16 alkyl, alkenyl, alkynyl, aryl, or aralkyl group, each of which may contain up to 4 heteroatoms. In another embodiment an R group may be comprised of C1-12 alkyl, alkenyl, alkynyl, aryl, or aralkyl group, each of which may contain up to 6 heteroatoms. In another embodiment an R group may be comprised of C1-12 alkyl, alkenyl, alkynyl, aryl, or aralkyl group, each of which may contain up to 4 heteroatoms. In another embodiment an R group may be comprised of C1-12 alkyl, alkenyl, alkynyl, aryl, or aralkyl group, each of which may contain up to 3 heteroatoms. In another embodiment an R group may be comprised of C1-8 alkyl, alkenyl, alkynyl, aryl, or aralkyl group, each of which may contain up to 4 heteroatoms. In another embodiment an R group may be comprised of C1-8 alkyl, alkenyl, alkynyl, aryl, or aralkyl group, each of which may contain up to 3 heteroatoms. In another embodiment an R group may be comprised of C1-8 alkyl, alkenyl, alkynyl, aryl, or aralkyl group, each of which may contain up to 2 heteroatoms. In another embodiment an R group may be comprised of C1-6 alkyl, alkenyl, alkynyl, aryl, or aralkyl group, each of which may contain up to 3 heteroatoms. In another embodiment an R group may be comprised of C1-6 alkyl, alkenyl, alkynyl, aryl, or aralkyl group, each of which may contain up to 2 heteroatoms.

In one embodiment an R group may be covalently bonded to another R group. In one embodiment two or more R groups may be covalently bonded to one or more R groups.

In one embodiment one or more R group may be covalently bonded to one or more adjacent R group, such as in a heterocyclic ring structure, as a non-limiting example. In one embodiment two or more R groups may be covalently bonded together, such as in a heterocyclic ring structure, as a non-limiting example. In one embodiment one or more R groups may form a heterocyclic ring structure with a covalently bonded nitrogen atom, phosphorus atom, or sulfur atom.

In one embodiment one or more R groups may form a ring structure. In another embodiment one or more R groups may form at least part of a ring structure. In another embodiment one or more R groups may be at least part of a ring structure. In another embodiment one or more R groups may comprise at least part of a ring structure.

In another embodiment one or more R groups may form a heterocyclic ring structure. In one embodiment one or more R groups may form at least part of a heterocyclic ring structure. In one embodiment one or more R groups may be at least part of a heterocyclic ring structure. In another embodiment one or more R groups may comprise at least part of a heterocyclic ring structure.

In one embodiment an NPS stabilizing moiety may comprise one or more ring structure. In one embodiment an NPS stabilizing moiety may comprise one or more heterocyclic ring structure. In one embodiment an NPS stabilizing moiety may comprise one or more nitrogen heterocycle.

In one embodiment an NPS stabilizing moiety may comprise one or more five membered ring structure. In one embodiment an NPS stabilizing moiety may comprise one or more heterocyclic five membered ring structure. As a non-limiting example an NPS stabilizing moiety may comprise one or more of the following ring structures or ring structure derivatives, including but not limited to: pyrrolidine, pyrrolidinium, pyrrole, imidazole, imidazolium, 3-pyrroline, 2-pyrroline, 2H-pyrrole, 1H-pyrrole, pyrazolidine, imidazolidine, 2-pyrazoline, 2-imidiazoline, pyrazole, pyrazoline, imidazoline, 1,2,4-triazole, 1,2,3-triazole, tetrazole, tetrahydrothiophene, thiophene, oxazole, isoxazole, isothiazole, thiazole, thiazoline, thiazolidine, isothiazolidine, 1,2-oxathiolane, 1,3-oxathiolane, 1,2,5-oxadiazole, 1,2,3-oxadiazole, 1,3,4-thiadiazole, 1,2,5-thiadiazole, 1,2,4-thiadiazole, 1,2,3-thiadiazole, sulfolane, sulfolene, phospholane, phosphole, 2,4-thiazolidinedione, succinimide, 2-oxazolidone, or hydantoin, or derivatives or combinations thereof. As a further non-limiting example an NPS stabilizing moiety may comprise one or more of the following ring structures or ring structure derivatives, including but not limited to: pyrrolidine, pyrrolidinium, pyrrole, pyrazolidine, imidazole, imidazolium, imidazoline, pyrazole, pyrazoline, thiolane, or thiophene, or derivatives or combinations thereof.

In one embodiment an NPS stabilizing moiety may comprise one or more six membered ring structure. In one embodiment an NPS stabilizing moiety may comprise one or more heterocyclic six membered ring structure. As a non-limiting example an NPS stabilizing moiety may comprise one or more of the following ring structures or ring structure derivatives, including but not limited to: piperidine, piperidinium, pyridine, pyridinium, piperazine, pyridazine, pyrimidine, pyrazine, diazinane, diazine, 1,2,4-triazine, 1,3,5-triazine, thiane, thiopyran, thiopyrylium, 2H-thiopyran, 4H-thiopyran, 1,3-dithiane, 1,4-dithiane, 1,3,5-trithiane, morpholine, morpholinium, 4H-1,2-oxazine, 2H-1,2-oxazine, 6H-1,2-oxazine, 4H-1,3-oxazine, 2H-1,3-oxazine, 6H-1,3-oxazine, 4H-1,4-oxazine, 2H-1,4-oxazine, thiomorpholine, thiazine, 4H-1,4-thiazine, 2H-1,2-thiazine, 6H-1,2-thiazine, 2H-1,4-thiazine, thiomorpholine dioxide, or derivatives or combinations thereof. As a further non-limiting example an NPS stabilizing moiety may comprise one or more of the following ring structures or ring structure derivatives, including but not limited to: piperidine, piperidinium, pyridine, pyridinium, piperazine, pyridazine, pyrimidine, pyrazine, diazinane, diazine, thiomorpholine, thiazine, morpholine, or morpholinium, derivatives or combinations thereof.

In one embodiment an NPS stabilizing moiety may comprise one or more seven membered ring structure. In one embodiment an NPS stabilizing moiety may comprise one or more heterocyclic seven membered ring structure. As a non-limiting example an NPS stabilizing moiety may comprise one or more of the following ring structures or ring structure derivatives, including but not limited to: diazepane, azepane, 2,3-dihydroazepine, 2,5-dihydroazepine, 4,5-dihydroazepine, azepine, 2H-azepine, 3H-azepine, 4H-azepine, 1,2-diazepine, 1,3-diazepine, 1,4-diazepine, thiepine, thiepane, thiazepine, 1,3-thiazepine, 1,4-thiazepine, benzothiazepine, or dibenzothiazepine, or derivatives or combinations thereof.

In one embodiment an NPS stabilizing moiety may comprise one or more eight membered ring structure. In one embodiment an NPS stabilizing moiety may comprise one or more heterocyclic eight membered ring structure. As a non-limiting example an NPS stabilizing moiety may comprise one or more of the following ring structures or ring structure derivatives, including but not limited to: azocane, azocine, or thiocane, or derivatives or combinations thereof.

In one embodiment an NPS stabilizing moiety may comprise one or more double bond. In on embodiment an NPS stabilizing moiety may comprise a resonance structure. In one embodiment an NPS stabilizing moiety may comprise one or more aromatic ring. As a non-limiting example an NPS stabilizing moiety may comprise one or more of the following ring structures or ring structure derivatives, including but not limited to: a pyrrole, indole, isoindole, thiophene, benzothiophene, benzo[c]thiophene, imidazole, benzimidazole, purine, pyrazole, indazole, oxazole, benzoxazole, isoxazole, benzisoxazole, thiazole, benzothiazole, pyridine, quinoline, isoquinoline, pyrazine, quinoxaline, acridine, pyrimidine, quinazoline, pyridazine, cinnoline, phthalazine, 1,2,3-triazine, 1,2,4-triazine, 1,3,5-triazine, or derivatives or combinations thereof. As a further non-limiting example an NPS stabilizing moiety may comprise one or more of the following ring structures or ring structure derivatives, including but not limited to: pyridine, pyridinium, pyrrole, pyrrolidinium, imidazole, imidazolium, thiazole, or thiophene, or derivatives or combinations thereof.

In one embodiment an NPS stabilizing moiety may comprise one or more of the following ring structures or ring structure derivatives: pyrrole, pyrrolidine, pyrrolidinium, imidazole, imidazolium, pyrazole, pyrazolium, piperidine, piperidinium, pyridine, pyridinium, diazinane, piperazine, diazine, pyrimidine, pyridazine, pyrazine, thiophene, thiazole, or tetrahydrothiophene, or derivatives or combinations thereof.

In one embodiment an NPS stabilizing moiety may not comprise a nucleic acid or nucleic acid base.

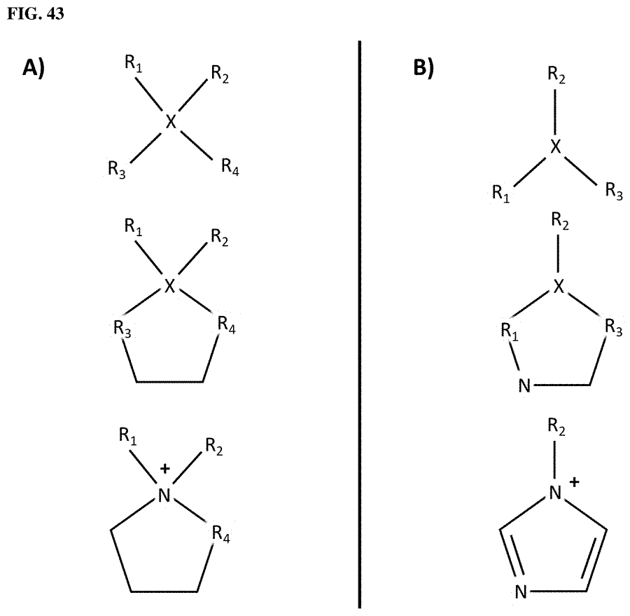

A non-limiting example of an NPS stabilizing moiety is shown in FIG. 43A. Wherein, X may be a nitrogen atom within a heterocyclic 5 membered ring covalently bonded to 4 carbon atoms represented by R₁, R₂, R₃, and R₄. In which R₁, R₂, R₃, and R₄ are independently selected R groups, as described herein, and R₁, R₂, R₃, and R₄ may be the same or different. Two of the nitrogen's carbon bonds are within the 5 membered ring, wherein R₃ may be covalently bonded to R₄, and R₃ and R₄ may form at least part of a heterocyclic ring structure. The nitrogen's other two carbon bonds are represented by R₁ and R₂, in which R₁ and R₂ are not covalently bonded to each other.

A non-limiting example of an NPS stabilizing moiety is shown in FIG. 43B. Wherein, X may be a nitrogen atom within an aromatic heterocyclic 5 membered ring covalently bonded to at least 3 carbon atoms represented by R₁, R₂, and R₃. In which R₁, R₂, and R₃ are independently selected R groups, as described herein, and R₁, R₂, and R₃ may be the same or different. Two of the nitrogen's carbon bonds are within the aromatic 5 membered ring, wherein R₁ may be covalently bonded to R₃, and R₁ and R₃ may form at least part of an aromatic heterocyclic ring structure. The nitrogen's other carbon bond is represented by R₂.

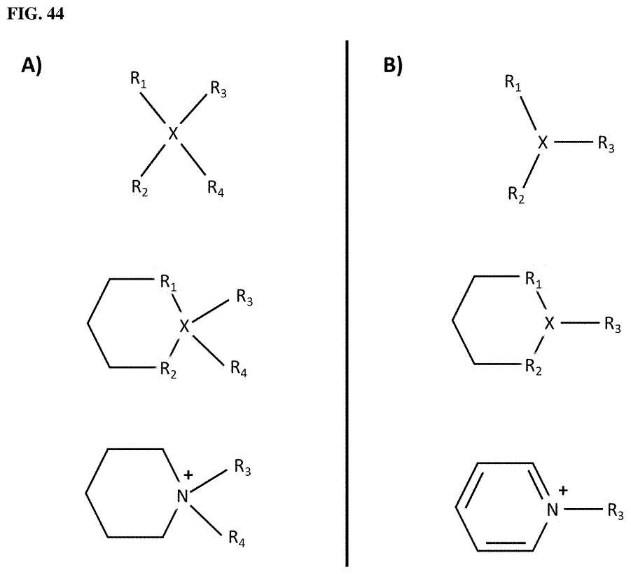

A non-limiting example of an NPS stabilizing moiety is shown in FIG. 44A. Wherein, X may be a nitrogen atom within a heterocyclic 6 membered ring covalently bonded to 4 carbon atoms represented by R₁, R₂, R₃, and R₄. In which R₁, R₂, R₃, and R₄ are independently selected R groups, as described herein, and R₁, R₂, R₃, and R₄ may be the same or different. Two of the nitrogen's carbon bonds are within the 6 membered ring, wherein R₁ may be covalently bonded to R₂, and R₁ and R₂ may form at least part of a heterocyclic ring structure. The nitrogen's other two carbon bonds are represented by R₃ and R₄, in which R₃ and R₄ are not covalently bonded to each other.

A non-limiting example of an NPS stabilizing moiety is shown in FIG. 44B. Wherein, X may be a nitrogen atom within an aromatic heterocyclic 6 membered ring covalently bonded to at least 3 carbon atoms represented by R₁, R₂, and R₃. In which R₁, R₂, and R₃ are independently selected R groups, as described herein, and R₁, R₂, and R₃ may be the same or different. Two of the nitrogen's carbon bonds are within the 6 membered ring, wherein R₁ may be covalently bonded to R₂, and R₁ and R₂ may form at least part of an aromatic heterocyclic ring structure.

Described herein is the COPS stabilizing moiety. In one embodiment of the present invention R_{B} may comprise a bridge of a stabilizing monomer connecting one or more NPS stabilizing moiety to one or more COPS stabilizing moiety, wherein R_{B} may be comprised of at least one carbon atom shared between an R group of an NPS stabilizing moiety and an R group of a COPS stabilizing moiety. Furthermore, R_{B} may be comprised of two R groups, one NPS R group and one COPS R group, covalently bonded together or shared between an NPS stabilizing moiety and a COPS stabilizing moiety. As a non-limiting example, a bridge may comprise a single carbon atom connecting an NPS stabilizing moiety to a COPS stabilizing moiety, wherein the NPS moiety and COPS moiety are covalently bonded and each share a single carbon atom.

In one embodiment one or more R group may comprise a linker R_{L}, wherein R_{L} may comprise at least one carbon atom connecting one or more NPS stabilizing moiety or one or more COPS stabilizing moiety to a stabilizing polymer. In one embodiment R_{L} may comprise a linker of a stabilizing monomer connecting one or more NPS stabilizing moiety or one or more COPS stabilizing moiety to a stabilizing polymer backbone.

In one embodiment an NPS stabilizing moiety may comprise one or more nitrogen atom with a positive charge at about physiologic pH. In one embodiment an NPS stabilizing moiety may comprise one or more a phosphorus atom with a positive charge at about physiologic pH. In one embodiment an NPS stabilizing moiety may comprise one or more sulfur atom with a positive charge at about physiologic pH. In one embodiment an NPS stabilizing moiety may comprise one or more aprotic moiety. In one embodiment an NPS stabilizing moiety may comprise one or more aprotic nitrogen moiety at about physiologic pH. In one embodiment an NPS stabilizing moiety may comprise one or more aprotic phosphorus moiety at about physiologic pH. In one embodiment an NPS stabilizing moiety may comprise one or more aprotic sulfur moiety at about physiologic pH.

In one embodiment an NPS stabilizing moiety may comprise one or more positive charge at about physiologic pH. In one embodiment an NPS stabilizing moiety may comprise one or more cation at about physiologic pH.

In one embodiment an NPS stabilizing moiety may comprise one or more PIF as described herein.

### COPS Stabilizing Moiety:

The inventors have discovered that RNA stabilizing substances may comprise carboxylic acid, carboxylate, carboxylate ester, organophosphate, phosphonic acid, phosphonate, sulfonic acid, sulfonate, or organosulfate, herein referred to as a COPS stabilizing moiety.

The inventors have also discovered that the stability of RNA substances may be enhanced in an RNA storage environment comprising a COPS stabilizing moiety, such as carboxylic acid, carboxylate, carboxylate ester, organophosphate, phosphonic acid, phosphonate, sulfonic acid, sulfonate, or organosulfate.

In one embodiment of the present invention a COPS stabilizing moiety may be comprised of a carbon atom, phosphorus atom, or sulfur atom. In another embodiment a COPS stabilizing moiety may be comprised of a carbon atom covalently bonded to at least two oxygen atoms. In another embodiment a COPS stabilizing moiety may be comprised of a phosphorus atom or sulfur atom covalently bonded to at least three oxygen atoms. In another embodiment a COPS stabilizing moiety may be comprised of a phosphorus atom or sulfur atom covalently bonded to at least four oxygen atoms.

In one embodiment a COPS stabilizing moiety may comprise carboxylic acid, carboxylate, carboxylate ester, organophosphate, sulfonic acid, or sulfonate, wherein the carboxylic acid, carboxylate, or carboxylate ester may comprise a carbon atom covalently bonded to 2 oxygen atoms, the organophosphate may comprise a phosphorus atom covalently bonded to 4 oxygen atoms, and the sulfonic acid or sulfonate may comprise a sulfur atom covalently bonded to 3 oxygen atoms.

In another embodiment a COPS stabilizing moiety may be comprised of phosphonic acid, phosphonate, or organosulfate, wherein the phosphonic acid or phosphonate may comprise a phosphorus atom covalently bonded to 3 oxygen atoms and the organosulfate may comprise a sulfur atom covalently bonded to 4 oxygen atoms.

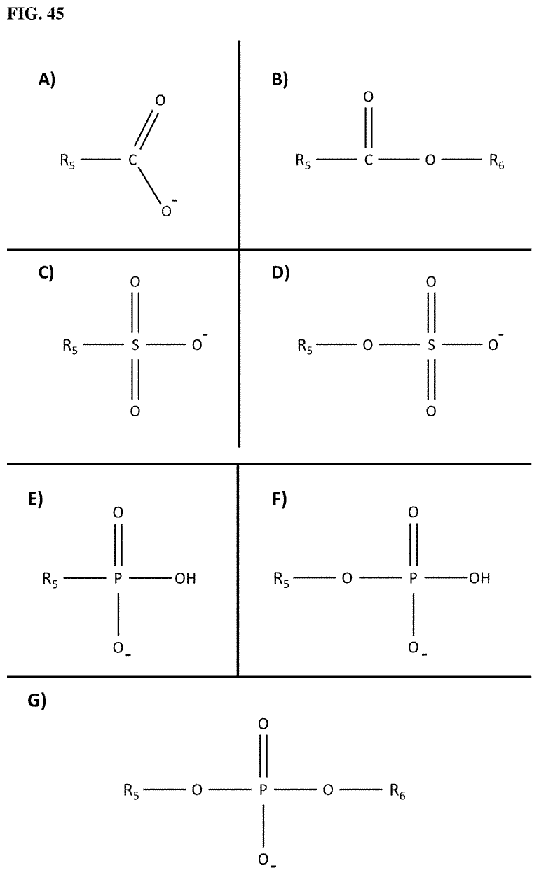

FIG. 45 shows non-limiting examples of COPS stabilizing moieties comprised of a carbon atom, phosphorus atom, or sulfur atom, wherein a COPS stabilizing moiety may be comprised of A) a carboxylate group B) a carboxylate ester C) a sulfonate group D) an organosulfate group E) a phosphonate group F) an organophosphate group bonded to one R group or G) an organophosphate group bonded to two R groups.

In one embodiment a COPS stabilizing moiety may be comprised of a substance according to FIG. 45A. Wherein C is a carbon atom covalently bonded to 2 oxygen atoms and an R group, represented by R₅, wherein R₅ is an independently selected R group as described herein. In another embodiment, R₅ may also be at least part of a bridge connecting one or more COPS stabilizing moiety to one or more NPS stabilizing moiety as described herein.

In one embodiment a COPS stabilizing moiety may be comprised of a substance according to FIG. 45B. Wherein C is a carbon atom covalently bonded to 2 oxygen atoms and one of the oxygen atoms is also covalently bonded to an R group, represented by R₅ and R₆. R₅ and R₆ may be independently selected R groups, as described herein, and R₅ and R₆ may be the same or different. In one embodiment, one or more of R₅ or R₆ may be interchangeable with one or more of the other R groups as described herein. As a non-limiting example, R₅ may be interchangeable with one or more other R group such as R₁, R₂, R₃, R₄, or R₆, as described herein, wherein R₅ may be replaced or exchange positions with one or more other R group such as R₁, R₂, R₃, R₄, or R₆. In another embodiment, R₅ may also be at least part of a bridge connecting one or more COPS stabilizing moiety to one or more NPS stabilizing moiety as described herein.

In one embodiment a COPS stabilizing moiety may be comprised of a substance according to FIG. 45C. Wherein S is a sulfur atom covalently bonded to 3 oxygen atoms and an R group represented by R₅, wherein R₅ is an independently selected R group as described herein. In another embodiment, R₅ may also be at least part of a bridge connecting one or more COPS stabilizing moiety to one or more NPS stabilizing moiety as described herein.

In one embodiment a COPS stabilizing moiety may be comprised of a substance according to FIG. 45D. Wherein S is a sulfur atom covalently bonded to 4 oxygen atoms and one or more of the oxygen atoms is also covalently bonded to an R group, represented by R₅, wherein R₅ is an independently selected R group as described herein. In another embodiment, R₅ may also be at least part of a bridge connecting one or more COPS stabilizing moiety to one or more NPS stabilizing moiety as described herein.

In one embodiment a COPS stabilizing moiety may be comprised of a substance according to FIG. 45E. Wherein P is a phosphorus atom covalently bonded to 3 oxygen atoms and an R group represented by R₅, wherein R₅ is an independently selected R group as described herein. In another embodiment, R₅ may also be at least part of a bridge connecting one or more COPS stabilizing moiety to one or more NPS stabilizing moiety as described herein.

In one embodiment a COPS stabilizing moiety may be comprised of a substance according to FIG. 45F. Wherein P is a phosphorus atom covalently bonded to 4 oxygen atoms and one or more of the oxygen atoms is also covalently bonded to an R group, represented by R₅, wherein R₅ is an independently selected R group as described herein. In another embodiment, R₅ may also be at least part of a bridge connecting one or more COPS stabilizing moiety to one or more NPS stabilizing moiety as described herein.

In one embodiment a COPS stabilizing moiety may be comprised of a substance according to FIG. 45G. Wherein P is a phosphorus atom covalently bonded to 4 oxygen atoms and one or more of the oxygen atoms is also covalently bonded to an R group, represented by R₅ and R₆. R₅ and R₆ may be independently selected R groups, as described herein, and R₅ and R₆ may be the same or different. In one embodiment, one or more of R₅ or R₆ may be interchangeable with one or more of the other R groups as described herein As a non-limiting example, R₅ may be interchangeable with one or more other R group such as R₁, R₂, R₃, R₄, or R₆, as described herein, wherein R₅ may be replaced or exchange positions with one or more other R group such as R₁, R₂, R₃, R₄, or R₆. In another embodiment, R₅ may also be at least part of a bridge connecting one or more COPS stabilizing moiety to one or more NPS stabilizing moiety as described herein.

In one embodiment a COPS stabilizing moiety may comprise one or more negative charge at about physiologic pH.

In one embodiment a COPS stabilizing moiety may comprise one or more PIF at about physiologic pH as described herein.

In one embodiment one or more R group may be covalently bonded to one or more other R group. In one embodiment two or more R groups may be covalently bonded to one or more other R groups.

### Stabilizing Monomer:

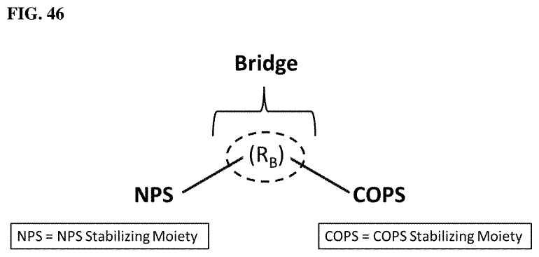

The inventors have discovered that stabilized RNA compositions or stabilized RNA mixtures may comprise stabilizing monomers comprising one or more NPS stabilizing moiety or one or more COPS stabilizing moiety. A non-limiting example diagram of a stabilizing monomer is shown in FIG. 46. Wherein, NPS is an NPS stabilizing moiety, COPS is a COPS stabilizing moiety, and the NPS stabilizing moiety and the COPS stabilizing moiety may be connected by a bridge, represented by R_{B}. The terms NPS stabilizing moiety and COPS stabilizing moiety are used herein to distinguish two moieties that may be connected, either directly, or by a bridge comprised of at least one carbon atom to characterize a class of monomers that may be used either as monomers or as components of polymers to stabilize RNA compositions or RNA mixtures comprising at least one or more RNA substance.

The term bridge, represented by R_{B}, refers to at least one carbon atom that may be shared between an R group of an NPS stabilizing moiety and an R group of a COPS stabilizing moiety. Furthermore, a bridge (R_{B}) may be comprised of two R groups, one NPS R group and one COPS R group, covalently bonded together or shared between an NPS stabilizing moiety and a COPS stabilizing moiety. As a non-limiting example, a bridge may comprise a single carbon atom connecting an NPS stabilizing moiety to a COPS stabilizing moiety, wherein the NPS moiety and COPS moiety are covalently bonded and each share a single carbon atom.

In one embodiment of the present invention R_{B} may comprise a bridge of a stabilizing monomer connecting one or more NPS stabilizing moiety to one or more COPS stabilizing moiety, wherein R_{B} may be comprised of at least one carbon atom shared between an R group of an NPS stabilizing moiety and an R group of a COPS stabilizing moiety.

In one embodiment one or more R group may comprise a linker R_{L}, wherein R_{L} may comprise at least one carbon atom connecting one or more NPS stabilizing moiety or one or more COPS stabilizing moiety to a stabilizing polymer. In one embodiment R_{L} may comprise a linker of a stabilizing monomer connecting one or more NPS stabilizing moiety or one or more COPS stabilizing moiety to at least part of a stabilizing polymer.

In one embodiment R_{L} may comprise a linker of a stabilizing monomer connecting one or more NPS stabilizing moiety or one or more COPS stabilizing moiety to at least part of a backbone chain of stabilizing polymer. In one embodiment R_{L} may comprise a linker of a stabilizing monomer connecting one or more NPS stabilizing moiety or one or more COPS stabilizing moiety to at least part of a backbone repeat unit of a stabilizing polymer.

In one embodiment R_{L} may comprise a linker of a stabilizing monomer covalently bonding one or more NPS stabilizing moiety or one or more COPS stabilizing moiety to at least part of a stabilizing polymer. In one embodiment R_{L} may comprise a linker of a stabilizing monomer covalently bonding one or more NPS stabilizing moiety or one or more COPS stabilizing moiety to at least part of a backbone chain of stabilizing polymer. In one embodiment R_{L} may comprise a linker of a stabilizing monomer covalently bonding one or more NPS stabilizing moiety or one or more COPS stabilizing moiety to at least part of a backbone repeat unit of a stabilizing polymer.

In one embodiment a stabilizing monomer may comprise one or more NPS stabilizing moiety and one or more COPS stabilizing moiety. In one embodiment a stabilizing monomer may comprise one or more NPS stabilizing moiety and one or more COPS stabilizing moiety connected by a bridge.

In one embodiment a stabilizing monomer may comprise a bridge.

In one embodiment of a stabilizing monomer a COPS stabilizing moiety may be absent. In one embodiment of a stabilizing monomer an NPS stabilizing moiety may be absent. In one embodiment of a stabilizing monomer a bridge may be absent. In one embodiment a stabilizing monomer may comprise one or more NPS stabilizing moiety without a COPS stabilizing moiety. In one embodiment a stabilizing monomer may comprise one or more COPS stabilizing moiety without an NPS stabilizing moiety.

FIG. 46 shows a non-limiting example diagram of a stabilizing monomer, wherein an NPS stabilizing moiety is connected to a COPS stabilizing moiety by a bridge comprised of at least one or more carbon atom, represented by R_{B}. Wherein, R_{B} is comprised of at least one or more R group as described herein.

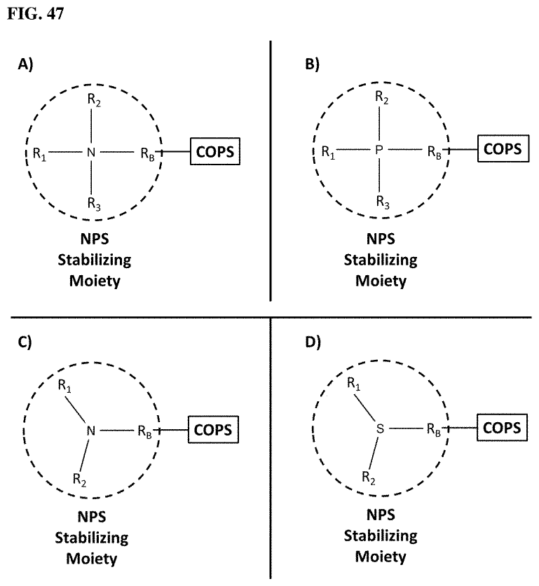

FIG. 47 illustrates non-limiting examples of stabilizing monomers. FIG. 47 shows non-limiting examples of an NPS stabilizing moiety comprised of a nitrogen atom, phosphorus atom, or sulfur atom connected to a COPS stabilizing moiety by a bridge, represented by R_{B}, wherein R_{B} is comprised of at least one or more R group as described herein. As a non-limiting example an NPS stabilizing moiety of a stabilizing monomer may be comprised of a A) nitrogen atom covalently bonded to four R groups, B) phosphorus atom covalently bonded to four R groups, C) nitrogen atom covalently bonded to three R groups, or D) sulfur atom covalently bonded to three R groups.

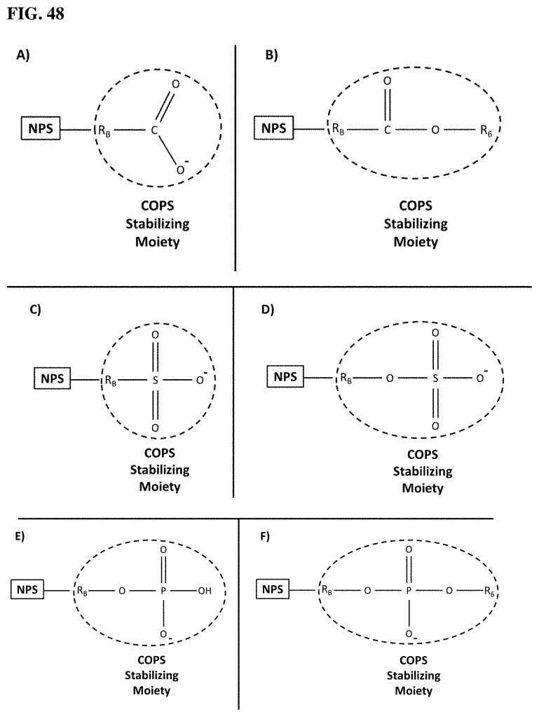

FIG. 48 illustrates non-limiting examples of stabilizing monomers. FIG. 48 shows non-limiting examples of a COPS stabilizing moiety comprised of a carbon atom, phosphorus atom, or sulfur atom connected to an NPS stabilizing moiety by a bridge, represented by R_{B}, wherein R_{B} is comprised of at least one or more R group as described herein. As a non-limiting example, a COPS stabilizing moiety of a stabilizing monomer may be comprised of A) a carboxylate group B) a carboxylate ester C) a sulfonate group D) an organosulfate group E) an organophosphate group bonded to one R group or F) an organophosphate group bonded to two R groups.

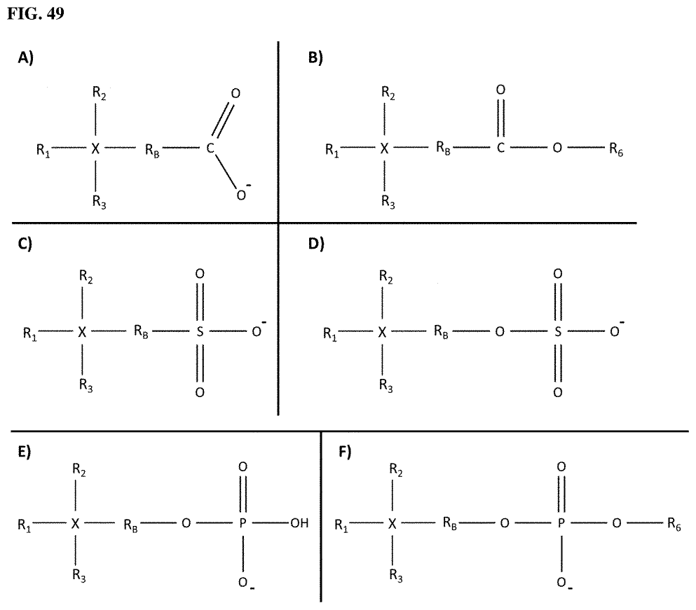

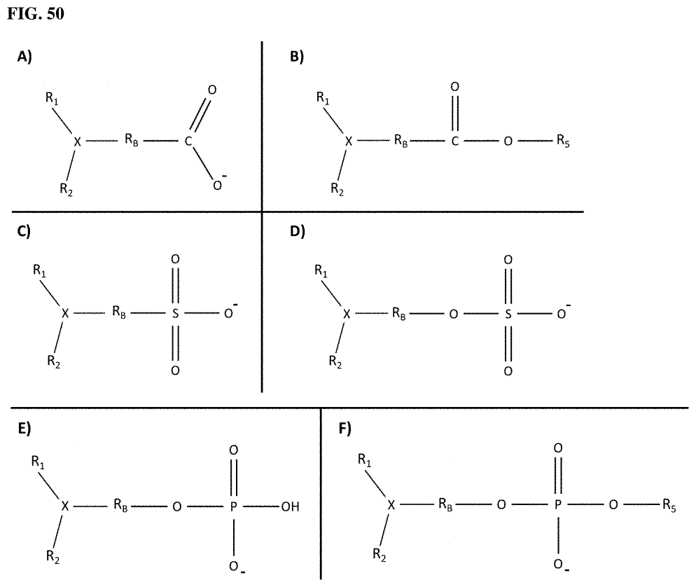

FIG. 49 and FIG. 50 illustrate non-limiting example structures of stabilizing monomers comprised of an NPS stabilizing moiety and a COPS stabilizing moiety connected by a bridge comprising at least one carbon atom, represented by R_{B}, wherein R_{B} is comprised of at least one or more R group as described herein.

As a non-limiting example FIG. 49 shows an NPS stabilizing moiety wherein X may be a nitrogen atom or phosphorus atom covalently bonded to four R groups and the NPS stabilizing moiety is connected by a bridge to a COPS stabilizing moiety comprised of A) a carboxylate group B) a carboxylate ester C) a sulfonate group D) an organosulfate group E) an organophosphate group bonded to one R group or F) an organophosphate group bonded to two R groups.

As a non-limiting example FIG. 50 shows an NPS stabilizing moiety wherein X may be a nitrogen atom or sulfur atom covalently bonded to three R groups and the NPS stabilizing moiety is connected by a bridge to a COPS stabilizing moiety comprised of A) a carboxylate group B) a carboxylate ester C) a sulfonate group D) an organosulfate group E) an organophosphate group bonded to one R group or F) an organophosphate group bonded to two R groups.

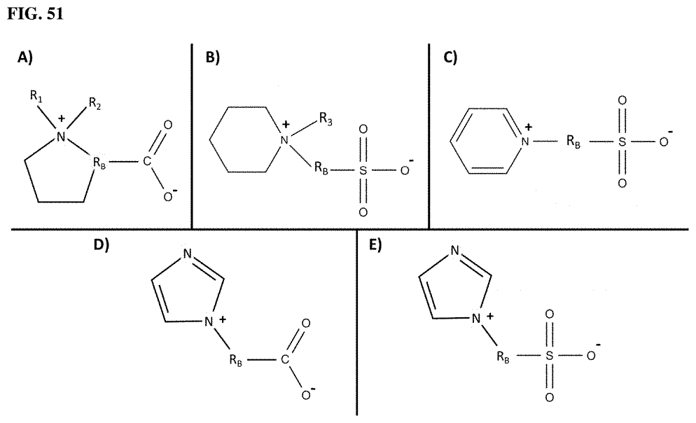

FIG. 51 illustrates non-limiting example structures of stabilizing monomers comprised of an NPS stabilizing moiety and a COPS stabilizing moiety, wherein an NPS stabilizing moiety may be comprised of a heterocyclic five membered ring or a heterocyclic six membered ring. The NPS and COPS stabilizing moiety are connected by a bridge comprising at least one carbon atom, represented by R_{B}, wherein R_{B} is comprised of at least one or more R group as described herein. As a non-limiting example an NPS stabilizing moiety may be comprised of nitrogen atom covalently bonded to either four R groups or covalently bonded to three R groups, such as the following non-limiting examples in FIG. 51 showing A) A stabilizing monomer comprising an NPS stabilizing moiety comprised of a heterocyclic five membered ring structure and a COPS stabilizing moiety comprised of a carboxylate group. B) A stabilizing monomer comprising an NPS stabilizing moiety comprised of a heterocyclic six membered ring structure and a COPS stabilizing moiety comprised of a sulfonate group. C) A stabilizing monomer comprising an NPS stabilizing moiety comprised of a heterocyclic aromatic six membered ring structure and a COPS stabilizing moiety comprised of a sulfonate group. D) A stabilizing monomer comprising an NPS stabilizing moiety comprised of a heterocyclic aromatic five membered ring structure and a COPS stabilizing moiety comprised of a carboxylate group. E) A stabilizing monomer comprising an NPS stabilizing moiety comprised of a heterocyclic aromatic five membered ring structure and a COPS stabilizing moiety comprised of a sulfonate group.

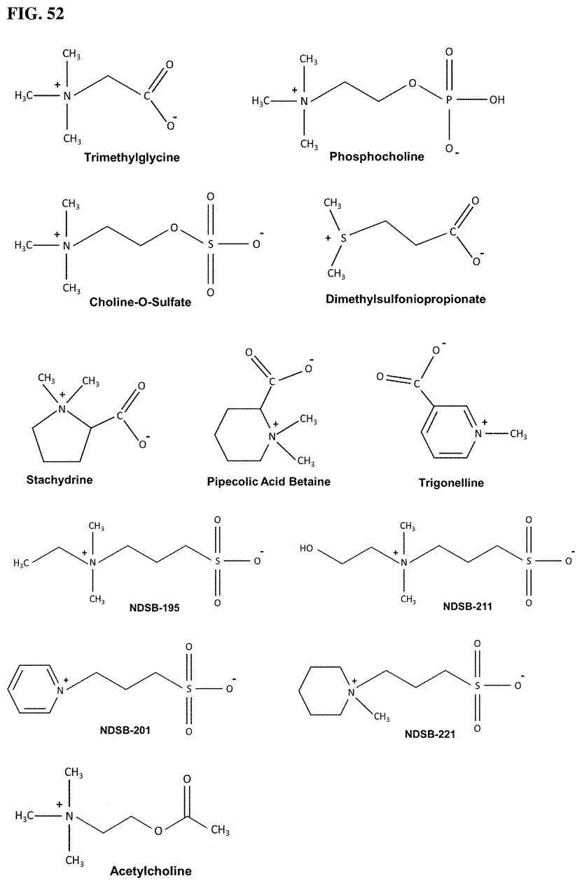

FIG. 52 shows non-limiting examples of stabilizing monomers comprised of an NPS stabilizing moiety and a COPS stabilizing moiety connected by a bridge comprising at least one carbon atom: TMG, phosphocholine, choline-O-sulfate, DMSP, stachydrine, pipecolic acid betaine, trigonelline, NDSB-195, NDSB-211, NDSB-201, NDSB-221, and acetylcholine.

In one embodiment one or more R group may be covalently bonded to one or more other R group. In one embodiment two or more R groups may be covalently bonded to one or more other R groups.

In one embodiment one or more R group may not be covalently bonded to one or more other R group. In one embodiment two or more R groups may not be covalently bonded to one or more other R groups.

In one embodiment one or more R group may be covalently bonded to one or more NPS stabilizing moiety or one or more COPS stabilizing moiety. In one embodiment one or more R group may covalently bond one or more NPS stabilizing moiety to one or more COPS stabilizing moiety.

In one embodiment one or more R group may be at least part of a stabilizing polymer. In one embodiment one or more R group may be at least part of one or more backbone chain of a stabilizing polymer. In one embodiment one or more R group may be at least part of one or more backbone repeat unit of a stabilizing polymer. In one embodiment one or more R group may be at least part of a sidechain, pendant group, or side group covalently bonded to at least part of one or more backbone chain of a stabilizing polymer. In one embodiment one or more R group may be at least part of a sidechain, pendant group, or side group covalently bonded to at least part of one or more backbone repeat unit of a stabilizing polymer.

In one embodiment one or more R group may be covalently bonded to at least part of a stabilizing polymer. In one embodiment one or more R group may be covalently bonded to at least part of one or more backbone chain of a stabilizing polymer. In one embodiment one or more R group may be covalently bonded to at least part of one or more backbone repeat unit of a stabilizing polymer. In one embodiment one or more R group may be covalently bonded to at least part of a sidechain, pendant group, or side group covalently bonded to at least part of one or more backbone chain of a stabilizing polymer. In one embodiment one or more R group may be covalently bonded to at least part of a sidechain, pendant group, or side group covalently bonded to at least part of one or more backbone repeat unit of a stabilizing polymer

In one embodiment one or more R group may covalently link one or more NPS stabilizing moiety or one or more COPS stabilizing moiety to at least part of a stabilizing polymer. In one embodiment one or more R group may covalently link one or more NPS stabilizing moiety or one or more COPS stabilizing moiety to at least part of one or more backbone chain of a stabilizing polymer. In one embodiment one or more R group may covalently link one or more NPS stabilizing moiety or one or more COPS stabilizing moiety to at least part of one or more one or more backbone repeat unit of a stabilizing polymer. In one embodiment one or more R group may covalently link one or more NPS stabilizing moiety or one or more COPS stabilizing moiety to at least part of one or more stabilizing polymer sidechain, pendant group, or side group covalently bonded to at least part of one or more backbone chain of a stabilizing polymer. In one embodiment one or more R group may covalently link one or more NPS stabilizing moiety or one or more COPS stabilizing moiety to at least part of one or more stabilizing polymer sidechain, pendant group, or side group covalently bonded to at least part of one or more backbone repeat unit of a stabilizing polymer.

In one embodiment one or more R group may covalently link one or more stabilizing monomer to at least part of a stabilizing polymer. In one embodiment one or more R group may covalently link one or more stabilizing monomer to at least part of one or more backbone chain of a stabilizing polymer. In one embodiment one or more R group may covalently link one or more stabilizing monomer to at least part of one or more one or more backbone repeat unit of a stabilizing polymer. In one embodiment one or more R group may covalently link one or more stabilizing monomer to at least part of one or more stabilizing polymer sidechain, pendant group, or side group covalently bonded to at least part of one or more backbone chain of a stabilizing polymer. In one embodiment one or more R group may covalently link one or more stabilizing monomer to at least part of one or more stabilizing polymer sidechain, pendant group, or side group covalently bonded to at least part of one or more backbone repeat unit of a stabilizing polymer.

In embodiment a stabilizing monomer may comprise one or more hydrolyzable bonds.

### Stabilizing Monomer Compositions:

The inventors have discovered that stabilizing monomers may stabilize RNA substances.

The inventors have discovered that RNA stabilizing substances may comprise stabilizing monomers. The inventors have also discovered that the stability of RNA substances may be enhanced in an RNA storage environment comprising one or more stabilizing monomer. The inventors have also discovered that the stability of RNA substances may be enhanced in an RNA storage environment comprising one or more stabilizing monomer and one or more additional RNA stabilizing substance, such as an aprotic substance.

The inventors have also discovered that the stability of RNA substances may be enhanced in compositions comprising one or more RNA substance and one or more stabilizing monomer. The inventors have also discovered that the stability of RNA substances may be enhanced in compositions comprising one or more RNA substance, one or more stabilizing monomer, and one or more additional RNA stabilizing substance, such as an aprotic substance.

In one embodiment of the present invention, one or more RNA stabilizing substance may comprise one or more stabilizing monomer.

One embodiment of the present invention may include a composition comprised of one or more RNA substance and one or more stabilizing monomer. Another embodiment of the present invention may include a composition comprised of one or more RNA substance, one or more stabilizing monomer, and one or more additional RNA stabilizing substance, such as an aprotic substance.

One embodiment of the present invention may include a combination or mixture comprising one or more RNA substance and one or more stabilizing monomer. Another embodiment of the present invention may include a combination or mixture comprising one or more RNA substance, one or more stabilizing monomer, and one or more additional RNA stabilizing substance, such as an aprotic substance.

Embodiments of the present invention that comprise one or more RNA substance and one or more stabilizing monomer may include combining, such as by mixing, one or more RNA substance with one or more substance that comprises at least one or more stabilizing monomer.

Embodiments of the present invention that comprise one or more RNA substance, one or more stabilizing monomer, and one or more additional RNA stabilizing substance, such as an aprotic substance, may include combining, such as by mixing, one or more RNA substance with one or more substance that comprises one or more stabilizing monomer and one or more substance that comprises one or more additional RNA stabilizing substance, such as an aprotic substance.

An embodiment of the present invention may include compositions of materials that comprise one or more RNA substance and one or more stabilizing monomer. The storage environment that improves the stability of RNA substances may comprise one or more vapor, liquid, powder, or solid stabilizing monomer.

An embodiment of the present invention may include compositions of materials that comprise one or more RNA substance, one or more stabilizing monomer, and one or more additional RNA stabilizing substance, such as an aprotic substance. The storage environment that improves the stability of RNA substances may comprise one or more vapor, liquid, powder, or solid stabilizing monomer and may comprise one or more vapor, liquid, powder, or solid additional RNA stabilizing substance, such as an aprotic substance.

An embodiment of the present invention may include combinations of materials that comprise one or more RNA substance and one or more stabilizing monomer. The storage environment that improves the stability of RNA substances may comprise one or more vapor, liquid, powder, or solid stabilizing monomer.

An embodiment of the present invention may include combinations of materials that comprise one or more RNA substance, one or more stabilizing monomer, and one or more additional RNA stabilizing substance, such as an aprotic substance. The storage environment that improves the stability of RNA substances may comprise one or more vapor, liquid, powder, or solid stabilizing monomer and may comprise one or more vapor, liquid, powder, or solid additional RNA stabilizing substance, such as an aprotic substance.

In one embodiment of the present invention a composition comprised of one or more RNA substance and one or more stabilizing monomer, produces a mixture with one or more RNA substance and one or more stabilizing monomer. In one embodiment of the present invention a composition comprised of one or more RNA substance, one or more stabilizing monomer, and one or more additional RNA stabilizing substance, such as an aprotic substance, produces a mixture with one or more RNA substance, one or more stabilizing monomer, and one or more additional RNA stabilizing substance, such as an aprotic substance.

In one embodiment of the present invention a combination comprising one or more RNA substance and one or more stabilizing monomer, produces a mixture with one or more RNA substance and one or more stabilizing monomer. In one embodiment of the present invention a combination comprising one or more RNA substance, one or more stabilizing monomer, and one or more additional RNA stabilizing substance, such as an aprotic substance, produces a mixture with one or more RNA substance, one or more stabilizing monomer, and one or more additional RNA stabilizing substance, such as an aprotic substance.

In one embodiment of the present invention a composition with improved RNA stability may comprise one or more RNA substance and one or more stabilizing monomer. In one embodiment of the present invention a composition with improved RNA stability may comprise one or more RNA substance, one or more stabilizing monomer, and one or more additional RNA stabilizing substance, such as an aprotic substance.

In one embodiment of the present invention a composition with improved RNA stability may comprise a mixture of one or more RNA substance and one or more stabilizing monomer. In one embodiment of the present invention a composition with improved RNA stability may comprise a mixture of one or more RNA substance, one or more stabilizing monomer, and one or more additional RNA stabilizing substance, such as an aprotic substance.

In one embodiment of the present invention, each component included in a composition comprising one or more RNA substance and one or more stabilizing monomer, may be stored separately, such as in a kit, or such as individual substances or as mixtures of one or more substance, and then combined later to produce a composition comprising one or more RNA substance and one or more stabilizing monomer.

In one embodiment of the present invention, each component included in a composition comprising one or more RNA substance, one or more stabilizing monomer, and one or more additional RNA stabilizing substance, such as an aprotic substance, may be stored separately, such as in a kit, or such as individually or as mixtures of one or more substance, and then combined later to produce a composition comprising one or more RNA substance, one or more stabilizing monomer, and one or more additional RNA stabilizing substance, such as an aprotic substance.

In one embodiment of the present invention a composition comprising one or more RNA substance and one or more stabilizing monomer may be at least partially biocompatible.

In one embodiment of the present invention a composition comprising one or more RNA substance, one or more stabilizing monomer, and one or more additional RNA stabilizing substance, such as an aprotic substance, may be at least partially biocompatible.

In one embodiment of the present invention a combination comprised of one or more RNA substance and one or more stabilizing monomer may be at least partially biocompatible.

In one embodiment of the present invention a combination comprised of one or more RNA substance, one or more stabilizing monomer, and one or more additional RNA stabilizing substance, such as an aprotic substance, may be at least partially biocompatible.

Embodiments of the present invention comprising one or more stabilizing monomers may include one or more forms of the stabilizing monomer. These forms may include, but are not limited to, one more counterions, ionic forms, conjugate bases, conjugate acids, protonated or deprotonated forms, hydrated or dehydrated forms, isomers, structural isomers, stereo isomers, chiral forms, salts, or combinations thereof.

In one embodiment a stabilizing monomer may comprise one or more of the following formulas:

(NPS)-R_{B}-(COPS), or (NPS), or (COPS)

Wherein NPS is an NPS stabilizing moiety as described herein, COPS is a COPS stabilizing moiety as described herein, and a bridge comprised of at least one carbon atom is represented by the R group R_{B}, as described herein.

Furthermore, NPS may comprise one or more of the following formulas:

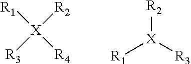

Wherein, X may be a nitrogen atom or phosphorus atom covalently bonded to 4 carbon atoms represented by R₁, R₂, R₃, and R₄ or X may be a nitrogen atom or sulfur atom covalently bonded to at least 3 carbon atoms represented by R₁, R₂, and R₃. Furthermore, each R group may be independently selected, as described herein, and may be the same or different.

Furthermore, COPS may comprise one or more of the following formulas:

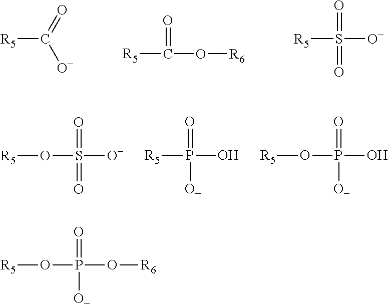

Wherein, C is a carbon atom, S is a sulfur atom, P is a phosphorus atom, and O is an oxygen atom and one or more oxygen atoms may be covalently bonded to one or more R group (represented by R₅ and R₆), as described herein. Furthermore, each R group may be independently selected, as described herein, and may be the same or different.

In one embodiment, one or more of R₁, R₂, R₃, R₄, R₅, or R₆ may be interchangeable with one or more of the other R groups as described herein. As a non-limiting example, R₆ may be interchangeable with one or more other R group such as R₁, R₂, R₃, R₄, or R₅, as described herein, wherein R₆ may be replaced or exchange positions with one or more other R group such as R₁, R₂, R₃, R₄, or R₅.

In one embodiment an R group may be comprised of at least one carbon atom. Wherein, R may be comprised of a C1-35 alkyl, alkenyl, alkynyl, aryl, or aralkyl group, each of which may contain up to 20 heteroatoms. In another embodiment an R group may be comprised of C1-30 alkyl, alkenyl, alkynyl, aryl, or aralkyl group, each of which may contain up to 16 heteroatoms. In another embodiment an R group may be comprised of C1-24 alkyl, alkenyl, alkynyl, aryl, or aralkyl group, each of which may contain up to 12 heteroatoms. In another embodiment an R group may be comprised of C1-20 alkyl, alkenyl, alkynyl, aryl, or aralkyl group, each of which may contain up to 10 heteroatoms. In another embodiment an R group may be comprised of C1-16 alkyl, alkenyl, alkynyl, aryl, or aralkyl group, each of which may contain up to 8 heteroatoms. In another embodiment an R group may be comprised of C1-16 alkyl, alkenyl, alkynyl, aryl, or aralkyl group, each of which may contain up to 6 heteroatoms. In another embodiment an R group may be comprised of C1-16 alkyl, alkenyl, alkynyl, aryl, or aralkyl group, each of which may contain up to 4 heteroatoms. In another embodiment an R group may be comprised of C1-12 alkyl, alkenyl, alkynyl, aryl, or aralkyl group, each of which may contain up to 6 heteroatoms. In another embodiment an R group may be comprised of C1-12 alkyl, alkenyl, alkynyl, aryl, or aralkyl group, each of which may contain up to 4 heteroatoms. In another embodiment an R group may be comprised of C1-12 alkyl, alkenyl, alkynyl, aryl, or aralkyl group, each of which may contain up to 3 heteroatoms. In another embodiment an R group may be comprised of C1-8 alkyl, alkenyl, alkynyl, aryl, or aralkyl group, each of which may contain up to 4 heteroatoms. In another embodiment an R group may be comprised of C1-8 alkyl, alkenyl, alkynyl, aryl, or aralkyl group, each of which may contain up to 3 heteroatoms. In another embodiment an R group may be comprised of C1-8 alkyl, alkenyl, alkynyl, aryl, or aralkyl group, each of which may contain up to 2 heteroatoms. In another embodiment an R group may be comprised of C1-6 alkyl, alkenyl, alkynyl, aryl, or aralkyl group, each of which may contain up to 3 heteroatoms. In another embodiment an R group may be comprised of C1-6 alkyl, alkenyl, alkynyl, aryl, or aralkyl group, each of which may contain up to 2 heteroatoms.

In one embodiment one or more R group may be covalently bonded to one or more other R group. In one embodiment two or more R groups may be covalently bonded to one or more other R groups.

In one embodiment one or more R group may be covalently bonded to one or more adjacent R group, such as in a heterocyclic ring structure as a non-limiting example. In one embodiment two or more R groups may be covalently bonded together, such as in a heterocyclic ring structure, as a non-limiting example. In one embodiment one or more R groups may form a heterocyclic ring structure with a covalently bonded nitrogen atom, phosphorus atom, or sulfur atom, such as a heterocyclic six membered or heterocyclic five membered ring structure as non-limiting examples.

In one embodiment one or more R groups may form a ring structure, such as a six membered or five membered ring structure as a non-limiting example. In another embodiment one or more R groups may form at least part of a ring structure, such as a six membered or five membered ring structure as non-limiting examples. In another embodiment one or more R groups may be at least part of a ring structure, such as a six membered or five membered ring structure as non-limiting examples. In another embodiment one or more R groups may comprise at least part of a ring structure, such as a six membered or five membered ring structure as non-limiting examples.

In another embodiment one or more R groups may form a heterocyclic ring structure, such as a heterocyclic six membered or heterocyclic five membered ring structure as non-limiting examples. In one embodiment one or more R groups may form at least part of a heterocyclic ring structure, such as a heterocyclic six membered or heterocyclic five membered ring structure as non-limiting examples. In one embodiment one or more R groups may be at least part of a heterocyclic ring structure, such as a heterocyclic six membered or heterocyclic five membered ring structure as non-limiting examples. In another embodiment one or more R groups may comprise at least part of a heterocyclic ring structure, such as a heterocyclic six membered or heterocyclic five membered ring structure as non-limiting examples.

In one embodiment an NPS stabilizing moiety may comprise one or more ring structure, such as a six membered or five membered ring structure as non-limiting examples. In one embodiment an NPS stabilizing moiety may comprise one or more heterocyclic ring structure, such as a heterocyclic six membered or heterocyclic five membered ring structure as non-limiting examples. In one embodiment an NPS stabilizing moiety may comprise one or more nitrogen heterocycle, such a six membered nitrogen heterocycle or five membered nitrogen heterocycle as non-limiting examples.

In one embodiment a stabilizing monomer may comprise one or more ring structure. In one embodiment a stabilizing monomer may comprise one or more heterocyclic ring structure. In one embodiment a stabilizing monomer may comprise one or more five membered ring structure. In one embodiment a stabilizing monomer may comprise one or more six membered ring structure. In one embodiment a stabilizing monomer may comprise one or more heterocyclic six membered ring structure. In one embodiment a stabilizing monomer may comprise one or more heterocyclic five membered ring structure. In one embodiment a stabilizing monomer may comprise one or more nitrogen heterocycles.

In one embodiment a stabilizing monomer may comprise one or more aromatic ring structure. In one embodiment a stabilizing monomer may comprise one or more aromatic heterocyclic ring structure. In one embodiment a stabilizing monomer may comprise one or more aromatic five membered ring structure. In one embodiment a stabilizing monomer may comprise one or more aromatic six membered ring structure. In one embodiment a stabilizing monomer may comprise one or more aromatic heterocyclic six membered ring structure. In one embodiment a stabilizing monomer may comprise one or more aromatic heterocyclic five membered ring structure. In one embodiment a stabilizing monomer may comprise one or more aromatic nitrogen heterocycles.

In one embodiment a stabilizing monomer may comprise one or more quaternary ammonium. In one embodiment a stabilizing monomer may comprise one or more tertiary amine. In one embodiment a stabilizing monomer may comprise one or more tertiary sulfonium. In one embodiment a stabilizing monomer may comprise one or more quaternary phosphonium.

In one embodiment a stabilizing monomer may comprise one or more quaternary ammonium cation or cationic moiety. In one embodiment a stabilizing monomer may comprise one or more tertiary sulfonium cation or cationic moiety. In one embodiment a stabilizing monomer may comprise one or more quaternary phosphonium cation or cationic moiety.

In one embodiment a stabilizing monomer may comprise one or more aliphatic quaternary ammonium. In one embodiment a stabilizing monomer may comprise one or more aliphatic tertiary amine. In one embodiment a stabilizing monomer may comprise one or more quaternary ammonium nitrogen heterocycle. In one embodiment a stabilizing monomer may comprise one or more quaternary ammonium aromatic nitrogen heterocycle. In one embodiment a stabilizing monomer may comprise one or more five membered quaternary ammonium nitrogen heterocycle. In one embodiment a stabilizing monomer may comprise one or more six membered quaternary ammonium nitrogen heterocycle. In one embodiment a stabilizing monomer may comprise one or more five membered aromatic quaternary ammonium nitrogen heterocycle. In one embodiment a stabilizing monomer may comprise one or more six membered aromatic quaternary ammonium nitrogen heterocycle.

In one embodiment a stabilizing monomer may comprise one or more cation or cationic moiety. In one embodiment a stabilizing monomer may comprise one or more anion or anionic moiety. In one embodiment a stabilizing monomer may comprise one or more zwitterion or zwitterionic moiety. In one embodiment a stabilizing monomer may be zwitterionic.

In one embodiment a stabilizing monomer may comprise one or more betaine. In one embodiment a stabilizing monomer may comprise one or more carboxybetaine. In one embodiment a stabilizing monomer may comprise one or more sulfobetaine, such as a non-detergent sulfobetaine as a non-limiting example.

In one embodiment a stabilizing monomer may comprise one or more quaternary ammonium cation that may be at least part of one or more nitrogen heterocycle or aromatic nitrogen heterocycle, such as a pyridinium, pyrrolidinium, imidazolium, piperidinium, indolium, pyrimidinium, or purinium group as non-limiting examples.

In one embodiment a stabilizing monomer may comprise one or more nitrogen heterocycle or aromatic nitrogen heterocycle, such as a pyridinium, pyrrolidinium, imidazolium, piperidinium, indolium, pyrimidinium, or purinium group as non-limiting examples.

In one embodiment a stabilizing monomer may comprise one or more carboxylate group. In one embodiment a stabilizing monomer may comprise one or more carboxylate ester. In one embodiment a stabilizing monomer may comprise one or more sulfonate group. In one embodiment a stabilizing monomer may comprise one or more organophosphate group. In one embodiment a stabilizing monomer may comprise one or more organosulfate group.

In one embodiment a stabilizing monomer may comprise one or more carboxylate anion or anionic moiety. In one embodiment a stabilizing monomer may comprise one or more sulfonate anion or anionic moiety. In one embodiment a stabilizing monomer may comprise one or more organophosphate anion or anionic moiety. In one embodiment a stabilizing monomer may comprise one or more organosulfate anion or anionic moiety.

In one embodiment a stabilizing monomer may comprise one or more pyridine or pyridinium group. In one embodiment a stabilizing monomer may comprise one or more pyrrolidine or pyrrolidinium group. In one embodiment a stabilizing monomer may comprise one or more imidazole or imidazolium group. In one embodiment a stabilizing monomer may comprise one or more piperidine or piperidinium group. In one embodiment a stabilizing monomer may comprise one or more pyrimidine or pyrimidinium group.

In one embodiment a stabilizing monomer may comprise one or more quaternary ammonium cation or tertiary amine that may be at least part of one or more nitrogen heterocycle, such as a pyridine, pyridinium, pyrrolidine, pyrrolidinium, pyrrole, imidazole, imidazolium, pyrazole, pyrimidine, pyrimidinium, piperidine, piperidinium as non-limiting examples.

In one embodiment a stabilizing monomer may not comprise a nucleic acid or nucleic base.

A non-limiting example of a stabilizing monomer, comprising an NPS stabilizing moiety comprised of a quaternary ammonium and a COPS stabilizing moiety comprised of a sulfonate group, that may be used is NDSB-195 (also known as dimethylethylammoniumpropane sulfonate or ethyl dimethyl ammonio propane sulfonate), wherein NDSB-195 may be substituted for or used in combination with one or more stabilizing monomer as described herein. One embodiment of the present invention may include combinations of substances comprising NDSB-195 and another embodiment may also comprise at least one RNA substance. Other embodiments may include combinations of substances comprising NDSB-195 wherein the concentration of NDSB-195 may be one or more concentration in the stabilizing substance weight percent concentration list or in the stabilizing substance molar concentration range list or in the stabilizing substance molar concentration list.

A non-limiting example of a stabilizing monomer, comprising an NPS stabilizing moiety comprised of a quaternary ammonium and a COPS stabilizing moiety comprised of an organophosphate group, that may be used is phosphocholine (also known as choline phosphate), wherein phosphocholine may be substituted for or used in combination with one or more stabilizing monomer as described herein. One embodiment of the present invention may include combinations of substances comprising phosphocholine and another embodiment may also comprise at least one RNA substance. Other embodiments may include combinations of substances comprising phosphocholine wherein the concentration of phosphocholine may be one or more concentration in the stabilizing substance weight percent concentration list or in the stabilizing substance molar concentration range list or in the stabilizing substance molar concentration list.

A non-limiting example of a stabilizing monomer, comprising an NPS stabilizing moiety comprised of a five membered ring structure and a quaternary ammonium and a COPS stabilizing moiety comprised of a carboxylate group, that may be used is stachydrine (also known as proline betaine), wherein stachydrine may be substituted for or used in combination with one or more stabilizing monomer as described herein. One embodiment of the present invention may include combinations of substances comprising stachydrine and another embodiment may also comprise at least one RNA substance. Other embodiments may include combinations of substances comprising stachydrine wherein the concentration of stachydrine may be one or more concentration in the stabilizing substance weight percent concentration list or in the stabilizing substance molar concentration range list or in the stabilizing substance molar concentration list.

A non-limiting example of a stabilizing monomer, comprising an NPS stabilizing moiety comprised of a quaternary ammonium and a COPS stabilizing moiety comprised of a carboxylate ester group, that may be used is acetylcholine wherein acetylcholine may be substituted for or used in combination with one or more stabilizing monomer as described herein. One embodiment of the present invention may include combinations of substances comprising acetylcholine and another embodiment may also comprise at least one RNA substance. Other embodiments may include combinations of substances comprising acetylcholine wherein the concentration of acetylcholine may be one or more concentration in the stabilizing substance weight percent concentration list or in the stabilizing substance molar concentration range list or in the stabilizing substance molar concentration list.

A non-limiting example of a stabilizing monomer, comprising an NPS stabilizing moiety comprised of an aromatic six membered ring structure and a COPS stabilizing moiety comprised of a sulfonate group, that may be used is 3-(1-pyridinio)-1-propane sulfonate (NDSB-201) (also known as NDSB-201), wherein NDSB-201 may be substituted for or used in combination with one or more stabilizing monomer as described herein. One embodiment of the present invention may include combinations of substances comprising NDSB-201 and another embodiment may also comprise at least one RNA substance. Other embodiments may include combinations of substances comprising NDSB-201 wherein the concentration of NDSB-201 may be one or more concentration in the stabilizing substance weight percent concentration list or in the stabilizing substance molar concentration range list or in the stabilizing substance molar concentration list.

A non-limiting example of a stabilizing monomer, comprising an NPS stabilizing moiety comprised of a quaternary ammonium and a COPS stabilizing moiety comprised of a carboxylate group, that may be used is trimethylglycine (TMG) (also known as glycine betaine or N,N,N-trimethylglycine), wherein TMG may be substituted for or used in combination with one or more stabilizing monomer as described herein. One embodiment of the present invention may include combinations of substances comprising TMG and another embodiment may also comprise at least one RNA substance. Other embodiments may include combinations of substances comprising TMG wherein the concentration of TMG may be one or more concentration in the stabilizing substance weight percent concentration list or in the stabilizing substance molar concentration range list or in the stabilizing substance molar concentration list.

A non-limiting example of a stabilizing monomer, comprising an NPS stabilizing moiety comprised of a six membered ring structure and a quaternary ammonium and a COPS stabilizing moiety comprised of a carboxylate group, that may be used is pipecolic acid betaine (also known as homostachydrine), wherein pipecolic acid betaine may be substituted for or used in combination with one or more stabilizing monomer as described herein. One embodiment of the present invention may include combinations of substances comprising pipecolic acid betaine and another embodiment may also comprise at least one RNA substance. Other embodiments may include combinations of substances comprising pipecolic acid betaine wherein the concentration of pipecolic acid betaine may be one or more concentration in the stabilizing substance weight percent concentration list or in the stabilizing substance molar concentration range list or in the stabilizing substance molar concentration list.

A non-limiting example of a stabilizing monomer, comprising an NPS stabilizing moiety comprised of a tertiary sulfonium and a COPS stabilizing moiety comprised of a carboxylate group, that may be used is dimethylsulfoniopropionate (DMSP), wherein DMSP may be substituted for or used in combination with one or more stabilizing monomer as described herein. One embodiment of the present invention may include combinations of substances comprising DMSP and another embodiment may also comprise at least one RNA substance. Other embodiments may include combinations of substances comprising DMSP wherein the concentration of DMSP may be one or more concentration in the stabilizing substance weight percent concentration list or in the stabilizing substance molar concentration range list or in the stabilizing substance molar concentration list.

A non-limiting example of a stabilizing monomer, comprising an NPS stabilizing moiety comprised of a quaternary ammonium and a COPS stabilizing moiety comprised of a organosulfate group, that may be used is choline-O-sulfate (COS) (also known as choline sulfate), wherein COS may be substituted for or used in combination with one or more stabilizing monomer as described herein. One embodiment of the present invention may include combinations of substances comprising COS and another embodiment may also comprise at least one RNA substance. Other embodiments may include combinations of substances comprising COS wherein the concentration of COS may be one or more concentration in the stabilizing substance weight percent concentration list or in the stabilizing substance molar concentration range list or in the stabilizing substance molar concentration list.

For conciseness, the following list of stabilizing monomers, is herein referred to as the stabilizing monomer list wherein one or more of the following substances may be substituted for or used in combination with one or more stabilizing monomer as described herein.

In other embodiments, other stabilizing monomers that may be substituted for or used in combination with one or more stabilizing monomer as described herein may comprise one or more of the following, including but not limited to: TMG, choline, phosphocholine, acetylcholine, carnitine, crotonobetaine, butyrobetaine, γ-butyrobetaine, t alanine betaine, β-alanine betaine, phosphobetaine, L-alpha-glycerylphosphorylcholine (alpha-GPC) glycerophosphorylcholine, glycerophosphocholine, COS, beta-dimethylsulfonopropionate, DMSP, hydroxyproline betaine, stachydrine, valine betaine, glutamic acid betaine, trigonelline, glutamine betaine, trimethyllysine, betaine esters, carboxybetaine esters 4-trimethyl-ammoniobutanoate, trimethyl-ammoniobutanoate, pipecolic acid betaine, sulfobetaines, non-detergent sulfobetaines, NDSB-195, NDSB-201, NDSB-209, dimethyl-2-hydroxyethylammoniumpropane sulfonate, (2-hydroxyethyl)dimethyl(3-sulfopropyl)ammonium, 3-[dimethyl-(2-hydroxyethyl)ammonio]-1-propanesulfonate, NDSB-211, 3-(1-methylpiperidinium)-1-propane sulfonate, NDSB-221, NDSB-223, NDSB-225, NDSB-249, dimethylbenzylammonium propane sulfonate, 3-(benzyldimethylammonio)propanesulfonate, NDSB-256, 3-(4-tert-butyl-1-pyridinio)-1-propanesulfonate, NDSB-256-4T, betaine aldehyde, imidazolium, piperidinium, pyridinium, pyrrolidinium, morpholinium, imidazolium betaine, piperidinium betaine, pyridinium betaine, pyrrolidinium betaine, morpholinium betaine, 4-dimethylsulphonio-2-hydroxybutyrate, 4-dimethylsulfonio-2-hydroxybutyrate, ethylmethylsulfoniopropionate, diethylsulfoniopropionate, isopropylmethylsulfoniopropionate, tetramethylenesulfoniopropionate, methylpropylsulfoniopropionate, 2-dimethylsulfonioacetate, 2-dimethylsulfonioethanol (dimethylthioethanol), dimethylsulfoniopropionate-aldehyde, methyl-dimethylsulfoniopropionate, S-Adenosyl methionine, S-Methylmethionine, (2-carboxy ethyl)(dimethyl)sulfonium, dimethyl(2-hydroxy-5-nitrobenzyl)sulfonium, dimethyl(phenacyl)sulfonium, (ethoxycarbonylmethyl)dimethylsulfonium, dibutylsulfoniopropionate butylethylsulfoniopropionate, butylmethylsulfoniopropionate, butylpropylsulfoniopropionate, diproprylsulfoniopropionate, diisopropylsulfoniopropionate, isopropylproprylsulfoniopropionate, isopropylethylsulfoniopropionate, isopropylbutylsulfoniopropionate, diethylsulfonio acetate, dibutylsulfonioacetate, ethylmethylsulfonio acetate, butylmethylsulfonioacetate, butylethylsulfonioacetate, dipropylsulfonioacetate, propylethylsulfonioacetate, porpylmethylsulfonioacetate, propylbutylsulfonioacetate, ispropylmethylsulfonioacetate, isopropylethylsulfonioacetate, isopropylbutylsulfonioacetate, isopropylpropylsulfonioacetate, diisopropylsulfonioacetate, sulfocholines, phosphatidylsulfocholines, carboxythetins, sulfothetins, sulfonium sulfonates, meldonium, arsenobetaine, betonicine, gamma-butyrobetaine, ergothioneine, propionobetaine, homostachydrine, phenylalanine betaine, homarine, isotrigonelline, taurine, dimethylthetin, hercynine, trigonelline methylester, trigonelline glucosylester, cocamidopropyl hydroxysultaine, cocamidopropyl betaine, trimethyl lysine, myristyl betaine, lauryl betaine, mesoionic compounds, mesomeric betaines, heterocyclic mesomeric betaines, acyclic mesomeric betaines, dimethylseleniopropionate, dimethyltelluriopropionate, pipecolic acid, pipecolate, 1,2-N-methylpipecolic acid, and 4-(3-butyl-1-imidazolio)-1-butanesulfonate, or derivatives or combinations thereof.

Embodiments of the present invention may include combinations comprising one or more of the substances from the stabilizing monomer list substituted for or used in combination with one or more stabilizing monomer or one or more RNA stabilizing substance, such as an aprotic substance as a non-limiting example, and other embodiments may also comprise at least one RNA substance.

Other embodiments may include combinations of substances comprising one or more stabilizing monomer selected from the stabilizing monomer list wherein the concentration of one or more substances may be one or more concentration in the stabilizing substance weight percent concentration list or in the stabilizing substance molar concentration range list or in the stabilizing substance molar concentration list.

In one embodiment a stabilizing monomer may comprise a carboxylic acid, carboxylate, carboxylate ester, organophosphate, phosphonic acid, phosphonate, sulfonic acid, sulfonate, or organosulfate.

In one embodiment a stabilizing monomer may comprise a carboxylate, carboxylate ester, organophosphate, or sulfonate.

In one embodiment a stabilizing monomer may comprise a quaternary ammonium, tertiary amine, quaternary phosphonium, or tertiary sulfonium.

In one embodiment a stabilizing monomer may comprise a carboxybetaine, carboxybetaine ester, betaine ester, sulfobetaine, or phosphobetaine.

In one embodiment, a stabilizing monomer may have a molecular weight between about 50-5,000 daltons. In one embodiment, a stabilizing monomer may have a molecular weight between about 50-2,000 daltons. In one embodiment, a stabilizing monomer may have a molecular weight between about 50-1,000 daltons. In one embodiment, a stabilizing monomer may have a molecular weight between about 50-900 daltons. In one embodiment, a stabilizing monomer may have a molecular weight between about 50-800 daltons. In one embodiment, a stabilizing monomer may have a molecular weight between about 50-700 daltons. In one embodiment, a stabilizing monomer may have a molecular weight between about 50-600 daltons. In one embodiment, a stabilizing monomer may have a molecular weight between about 50-500 daltons. In one embodiment, a stabilizing monomer may have a molecular weight between about 50-400 daltons. In one embodiment, a stabilizing monomer may have a molecular weight between about 50-300 daltons. In one embodiment, a stabilizing monomer may have a molecular weight between about 50-250 daltons.

In one embodiment, a stabilizing monomer may have a molecular weight between about 100-5,000 daltons. In one embodiment, a stabilizing monomer may have a molecular weight between about 100-2,000 daltons. In one embodiment, a stabilizing monomer may have a molecular weight between about 100-1,000 daltons. In one embodiment, a stabilizing monomer may have a molecular weight between about 100-900 daltons. In one embodiment, a stabilizing monomer may have a molecular weight between about 100-800 daltons. In one embodiment, a stabilizing monomer may have a molecular weight between about 100-700 daltons. In one embodiment, a stabilizing monomer may have a molecular weight between about 100-600 daltons. In one embodiment, a stabilizing monomer may have a molecular weight between about 100-500 daltons. In one embodiment, a stabilizing monomer may have a molecular weight between about 100-400 daltons. In one embodiment, a stabilizing monomer may have a molecular weight between about 100-300 daltons. In one embodiment, a stabilizing monomer may have a molecular weight between about 100-250 daltons.

One embodiment of the present invention is the method whereby one or more stabilizing monomer may be combined, such as by mixing, with at least one or more RNA substance to produce a mixture comprising at least one or more stabilizing monomer and at least one or more RNA substance. As a non-limiting example one or more RNA substance may be mixed with one or more stabilizing monomer.

Another embodiment of the present invention is the method whereby one or more stabilizing monomer may be combined, such as by mixing, with at least one or more RNA substance to produce a mixture comprising at least one or more RNA substance and at least one or more stabilizing monomer at one or more of the RNA stabilizing substance concentrations within the stabilizing substance molar concentration range list or in the stabilizing substance molar concentration list or the stabilizing substance weight percent concentration list as described herein. These same methods may be used to combine one or more other substances, including, but not limited to one or more cellular uptake agents or one or more additional RNA stabilizing substances, with one or more RNA substances and one or more stabilizing monomers to produce a mixture comprising one or more RNA substance, one or more stabilizing monomer, and one or more cellular uptake agent or one or more additional RNA stabilizing substance.

One embodiment of the present invention is the method whereby one or more stabilizing monomer may be combined, such as by mixing, with at least one or more RNA substance to produce a composition comprising at least one or more RNA substance and at least one or more stabilizing monomer. Another embodiment of the present invention is the method whereby one or more stabilizing monomer may be combined, such as by mixing, with one or more RNA substance to produce a composition comprising at least one or more RNA substance and at least one or more stabilizing monomer at one or more of the RNA stabilizing substance concentrations within the stabilizing substance molar concentration range list or in the stabilizing substance molar concentration list or stabilizing substance weight percent concentration list as described herein. These same methods may be used to combine one or more other substances, including, but not limited to one or more cellular uptake agents or one or more additional RNA stabilizing substance, with one or more RNA substance and one or more stabilizing monomer to produce a composition comprising one or more RNA substance, one or more stabilizing monomer, and one or more cellular uptake agent or one or more additional RNA stabilizing substance.

In embodiment a stabilizing monomer may comprise one or more hydrolyzable bonds.

In one embodiment a stabilizing monomer may comprise a cationic moiety at about physiologic pH. In one embodiment a stabilizing monomer may comprise an anionic moiety at about physiologic pH. In one embodiment a stabilizing monomer may comprise a zwitterion at about physiologic pH.

In one embodiment a stabilizing monomer may be cationic at about physiologic pH. In one embodiment a stabilizing monomer may be anionic at about physiologic pH. In one embodiment a stabilizing monomer may be zwitterionic at about physiologic pH.

In one embodiment a stabilizing monomer may comprise one or more PIF as described herein.

In one embodiment a polymer may be comprised of at least one or more stabilizing monomer.

In one embodiment, one or more stabilizing monomer may be at least part of a polymer comprised of at least one or more stabilizing monomer.

In one embodiment one or more composition comprising one or more RNA substance and one or more stabilizing monomer may comprise one or more water concentrations in the water concentration list or in the water concentration range list. In one embodiment one or more composition comprising one or more RNA substance, one or more stabilizing monomer, and one or more additional substance, such as one or more cellular uptake agent or one or more additional RNA stabilizing substance as non-limiting examples, may comprise one or more water concentrations in the water concentration list or in the water concentration range list. In one embodiment one or more composition comprising one or more RNA substance and one or more stabilizing monomer may have a dielectric constant of one or more dielectric constant in the dielectric constant list or in the dielectric constant range list. In one embodiment one or more composition comprising one or more RNA substance, one or more stabilizing monomer, and one or more additional substance, such as one or more cellular uptake agent or one or more additional RNA stabilizing substance as non-limiting examples, may have a dielectric constant of one or more dielectric constant in the dielectric constant list or in the dielectric constant range list.

Embodiments of the present invention may include compositions comprising one or more stabilizing monomer used in a composition comprising one or more RNA substance, one or more stabilizing monomer, and one or more of the following substances: water, cellular uptake agents, aprotic substances, low dielectric substances, cyclic phosphate or metaphosphate containing substances, non-carbohydrate organic osmolyte substances, tertiary sulfonium containing substances, quaternary ammonium containing substances, quaternary phosphonium containing substances, hydrotrope containing substances, surfactant containing substances, betaine containing substances, stabilizing polymers, or supplemental RNA stabilizing substances. Other embodiments may include one or more composition comprising one or more stabilizing monomer and one or more RNA substance and may also comprise one or more cellular uptake agent or one or more additional RNA stabilizing substances. Other embodiments may include compositions comprising one or more stabilizing monomer, one or more RNA substance, and one or more additional substances, such as: one or more cellular uptake agent, or one or more additional RNA stabilizing substance, or one or more buffering agent, or one or more chelating agent, or one or more inorganic salt, or water, as non-limiting examples.

Embodiments of the present invention may include one or more compositions comprising one or more RNA substance and one or more stabilizing monomer, wherein a composition is not lyophilized.

In one embodiment one or more composition comprising one or more RNA substance and one or more stabilizing monomer may not be lyophilized. In one embodiment one or more composition comprising one or more RNA substance, one or more stabilizing monomer, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more stabilizing monomer, wherein a composition may be lyophilized.

In one embodiment one or more composition comprising one or more RNA substance and one or more stabilizing monomer may be lyophilized. In one embodiment one or more composition comprising one or more RNA substance, one or more stabilizing monomer, and one or more additional substance, such as one or more cellular uptake agent or one or more additional RNA stabilizing substance as non-limiting examples, may be lyophilized.

In one embodiment a stabilizing monomer is not a lipid. In one embodiment a stabilizing monomer is not a cholesterol. In one embodiment a stabilizing monomer is not a saccharide.

In one embodiment a stabilizing monomer may not be a detergent. In one embodiment a stabilizing monomer may not be a peptide. In one embodiment a stabilizing monomer may not be a nucleic acid base. In one embodiment a stabilizing monomer may not be a nucleic acid.

Embodiments of the present invention may include one or more compositions comprising one or more stabilizing monomer and one or more RNA substance described herein, wherein one or more composition described herein may have a melting of one or more melting point in the stabilizing composition melting point list.

In one embodiment one or more composition comprising one or more RNA substance and one or more stabilizing monomer may have a melting point of one or more melting point in the stabilizing composition melting point list. In one embodiment one or more composition comprising one or more RNA substance, one or more stabilizing monomer, and one or more additional substance, such as one or more cellular uptake agent or one or more additional RNA stabilizing substance as a non-limiting example, may have a melting point of one or more melting point in the stabilizing composition melting point list.

### Stabilizing Polymer:

The inventors have discovered that RNA stabilizing substances may comprise stabilizing polymers, wherein a stabilizing polymer may be comprised of one or more stabilizing monomers.

As used herein a backbone chain is a series of covalently bonded atoms in a polymer that together create one or more continuous chains of a polymer.

As used herein a backbone repeat unit is the part of a polymer whose repetition would produce one or more backbone chains of a polymer, not including end groups, by covalently linking each repeat unit together successively along a backbone chain of a polymer.

In one embodiment a stabilizing polymer may be comprised of one or more backbone repeat unit, wherein the backbone repeat units may be the same or different. In other embodiments a stabilizing polymer may be comprised of two or more backbone repeat units, wherein the backbone repeat units may be the same or different.

In one embodiment a stabilizing polymer may be comprised of one or more type of backbone repeat unit. In one embodiment a stabilizing polymer may be comprised of two or more types of backbone repeat units, such as in a copolymer as a non-limiting example. In other embodiments a stabilizing polymer may comprise three or more types of backbone repeat units, four or more types of backbone repeat units, five or more types of backbone repeat units, ten or more types of backbone repeat units, twenty or more types of backbone repeat units, fifty or more types of backbone repeat units, or one hundred or more types of backbone repeat units.

In one embodiment a backbone chain of a stabilizing polymer may be comprised of one or more backbone repeat unit, wherein the backbone repeat units may be the same or different. In other embodiments a backbone chain of a stabilizing polymer may be comprised of two or more backbone repeat units, wherein the backbone repeat units may be the same or different.

In one embodiment a backbone chain of a stabilizing polymer may be comprised of one or more type of backbone repeat unit. In one embodiment a backbone chain of a stabilizing polymer may be comprised of two or more types of backbone repeat units. In other embodiments a backbone chain of a stabilizing polymer may comprise three or more types of backbone repeat units, four or more types of backbone repeat units, five or more types of backbone repeat units, ten or more types of backbone repeat units, twenty or more types of backbone repeat units, fifty or more types of backbone repeat units, or one hundred or more types of backbone repeat units.

In one embodiment a stabilizing polymer may be comprised of a copolymer, such as a bipolymer, terpolymer, or quaterpolymer, or higher order copolymer. In one embodiment a stabilizing polymer may be comprised of one or more the following types of copolymers, including, but not limited to: a linear copolymer, a random copolymer, an alternating copolymer, a statistical copolymer, a gradient copolymer, a periodic copolymer, a sequential copolymer, a block copolymer, a graft copolymer, a crosslinked copolymer, or a star copolymer, or combinations thereof.

In one embodiment a stabilizing polymer may be comprised of one or more backbone chain, wherein the backbone chains may be the same or different. In other embodiments a stabilizing polymer may be comprised of two or more backbone chains, wherein the backbone chains may be the same or different.

In one embodiment a stabilizing polymer may be comprised of one or more type of backbone chain. In one embodiment a stabilizing polymer may be comprised of two or more types of backbone chains, such as in a block copolymer as a non-limiting example. In other embodiments a stabilizing polymer may comprise three or more types of backbone chains, four or more types of backbone chains, five or more types of backbone chains, ten or more types of backbone chains, twenty or more types of backbone chains, fifty or more types of backbone chains, or one hundred or more types of backbone chains.

In one embodiment a stabilizing polymer may be comprised of a block copolymer, wherein a stabilizing polymer may be comprised of a diblock polymer, triblock polymer, quaterblock polymer, or higher order multiblock polymer. In one embodiment a stabilizing polymer may be comprised of a block copolymer, wherein a block copolymer may be comprised of one or more blocks. In one embodiment a stabilizing polymer may be comprised of a block copolymer, wherein a block copolymer may be comprised of two or more types of blocks. In other embodiments a stabilizing polymer may be comprised of three or more types of blocks, four or more types of blocks, five or more types of blocks, ten or more types of blocks, twenty or more types of blocks, fifty or more types of blocks, or one hundred or more types of blocks.

In one embodiment a stabilizing polymer may be comprised of a linear block copolymer. In one embodiment a stabilizing polymer may be comprised of a random block copolymer. In one embodiment a stabilizing polymer may be comprised of an alternating block copolymer.

In one embodiment a stabilizing polymer may be a homopolymer. In one embodiment a stabilizing polymer may comprise a homopolymer. In one embodiment a stabilizing polymer may be linear. In one embodiment a stabilizing polymer may be branched. In one embodiment a stabilizing polymer may comprise one or more branches. In one embodiment a stabilizing polymer may comprise one or more grafts.

In one embodiment a stabilizing polymer may comprise a copolymer. In one embodiment a stabilizing polymer may comprise a linear copolymer. In one embodiment a stabilizing polymer may be comprised of a linear copolymer. In one embodiment a stabilizing polymer may be comprised of a branched copolymer. In one embodiment a stabilizing polymer may be comprised of a graft copolymer. In one embodiment a stabilizing polymer may be comprised of a star copolymer.

In one embodiment a stabilizing polymer may be comprised of one or more sidechains, pendant groups, or side groups, wherein the sidechains, pendant groups, or side groups may be the same or different. In one embodiment a stabilizing polymer may be comprised of two or more sidechains, pendant groups, or side groups, wherein the sidechains, pendant groups, or side groups may be the same or different.

In one embodiment a stabilizing polymer may be comprised of one or more type of sidechain, pendant group, or side group. In one embodiment a stabilizing polymer may be comprised of two or more types of sidechains, pendant groups, or side groups. In other embodiments a stabilizing polymer may be comprised three or more types of sidechains, pendant groups, or side groups, four or more types of sidechains, pendant groups, or side groups, five or more types of sidechains, pendant groups, or side groups, ten or more types of sidechains, pendant groups, or side groups, twenty or more types of sidechains, pendant groups, or side groups, fifty or more types of sidechains, pendant groups, or side groups, or one hundred or more types of sidechains, pendant groups, or side groups.

In one embodiment a stabilizing polymer may be comprised of one or more stabilizing monomers, wherein the stabilizing monomers may be the same or different. In one embodiment a stabilizing polymer may be comprised of two or more stabilizing monomers, wherein the stabilizing monomers may be the same or different.

In one embodiment a stabilizing polymer may be comprised of one or more type of stabilizing monomer. In one embodiment a stabilizing polymer may be comprised of two or more types of stabilizing monomers. In other embodiments a stabilizing polymer may be comprised of three or more types of stabilizing monomers, four or more types of stabilizing monomers, five or more types of stabilizing monomers, ten or more types of stabilizing monomers, twenty or more types of stabilizing monomers, fifty or more types of stabilizing monomers, or one hundred or more types of stabilizing monomers.

In one embodiment a backbone chain of a stabilizing polymer may be comprised of one or more sidechains, pendant groups, or side groups, wherein the sidechains, pendant groups, or side groups may be the same or different. In one embodiment a backbone chain of a stabilizing polymer may be comprised of two or more sidechains, pendant groups, or side groups, wherein the sidechains, pendant groups, or side groups may be the same or different.

In one embodiment a backbone chain of a stabilizing polymer may be comprised of one or more type of sidechain, pendant group, or side group. In one embodiment a backbone chain of a stabilizing polymer may be comprised of two or more types of sidechains, pendant groups, or side groups. In other embodiments a backbone chain of a stabilizing polymer may be comprised three or more types of sidechains, pendant groups, or side groups, four or more types of sidechains, pendant groups, or side groups, five or more types of sidechains, pendant groups, or side groups, ten or more types of sidechains, pendant groups, or side groups, twenty or more types of sidechains, pendant groups, or side groups, fifty or more types of sidechains, pendant groups, or side groups, or one hundred or more types of sidechains, pendant groups, or side groups.

In one embodiment a backbone chain of a stabilizing polymer may be comprised of one or more stabilizing monomers, wherein the stabilizing monomers may be the same or different. In one embodiment a backbone chain of a stabilizing polymer may be comprised of two or more stabilizing monomers, wherein the stabilizing monomers may be the same or different.

In one embodiment a backbone chain of a stabilizing polymer may be comprised of one or more type of stabilizing monomer. In one embodiment a backbone chain of a stabilizing polymer may be comprised of two or more types of stabilizing monomers. In other embodiments a backbone chain of a stabilizing polymer may be comprised of three or more types of stabilizing monomers, four or more types of stabilizing monomers, five or more types of stabilizing monomers, ten or more types of stabilizing monomers, twenty or more types of stabilizing monomers, fifty or more types of stabilizing monomers, or one hundred or more types of stabilizing monomers.

In one embodiment a stabilizing polymer may be comprised of a copolymer or block copolymer, wherein the copolymer or block copolymer may be comprised of one or more type of backbone repeat unit. In one embodiment a stabilizing polymer may be comprised of a copolymer or block copolymer, wherein the copolymer or block copolymer may be comprised of two or more types of backbone repeat units. In other embodiments a stabilizing polymer may be comprised of a copolymer or block copolymer, wherein the copolymer or block copolymer may be comprised of three or more types of backbone repeat units, four or more types of backbone repeat units, five or more types of backbone repeat units, ten or more types of backbone repeat units, twenty or more types of backbone repeat units, fifty or more types of backbone repeat units, or one hundred or more types of backbone repeat units.

In one embodiment a stabilizing polymer may be comprised of a copolymer or block copolymer, wherein the copolymer or block copolymer may be comprised of one or more type of backbone chain. In one embodiment a stabilizing polymer may be comprised of a copolymer or block copolymer, wherein the copolymer or block copolymer may be comprised of two or more types of backbone chains. In other embodiments a stabilizing polymer may be comprised of a copolymer or block copolymer, wherein the copolymer or block copolymer may be comprised of three or more types of backbone chains, four or more types of backbone chains, five or more types of backbone chains, ten or more types of backbone chains, twenty or more types of backbone chains, fifty or more types of backbone chains, or one hundred or more types of backbone chains.

In one embodiment a stabilizing polymer may be comprised of a copolymer or block copolymer, wherein the copolymer or block copolymer may be comprised of one or more type of monomer. In one embodiment a stabilizing polymer may be comprised of a copolymer or block copolymer, wherein the copolymer or block copolymer may be comprised of two or more types of monomers. In other embodiments a stabilizing polymer may be comprised of a copolymer or block copolymer, wherein the copolymer or block copolymer may be comprised of three or more types of monomers, four or more types of monomers, five or more types of monomers, ten or more types of monomers, twenty or more types of monomers, fifty or more types of monomers, or one hundred or more types of monomers.

In one embodiment a stabilizing polymer may be comprised of a copolymer or block copolymer, wherein the copolymer or block copolymer may be comprised of one or more type of stabilizing monomer. In one embodiment a stabilizing polymer may be comprised of a copolymer or block copolymer, wherein the copolymer or block copolymer may be comprised of two or more types of stabilizing monomers. In other embodiments a stabilizing polymer may be comprised of a copolymer or block copolymer, wherein the copolymer or block copolymer may be comprised of three or more types of stabilizing monomers, four or more types of stabilizing monomers, five or more types of stabilizing monomers, ten or more types of stabilizing monomers, twenty or more types of stabilizing monomers, fifty or more types of stabilizing monomers, or one hundred or more types of stabilizing monomers.

In one embodiment a stabilizing polymer may be comprised of a copolymer or block copolymer, wherein the copolymer or block copolymer may be comprised of one or more type of sidechains, pendant groups, or side groups. In one embodiment a stabilizing polymer may be comprised of a copolymer or block copolymer, wherein the copolymer or block copolymer may be comprised of two or more types of sidechains, pendant groups, or side groups. In other embodiments a stabilizing polymer may be comprised of a copolymer or block copolymer, wherein the copolymer or block copolymer may be comprised of three or more types of sidechains, pendant groups, or side groups, four or more types of sidechains, pendant groups, or side groups, five or more types of sidechains, pendant groups, or side groups, ten or more types of sidechains, pendant groups, or side groups, twenty or more types of sidechains, pendant groups, or side groups, fifty or more types of sidechains, pendant groups, or side groups, or one hundred or more types of sidechains, pendant groups, or side groups.

In one embodiment one or more stabilizing polymer may comprise one or more sidechains, pendant groups, or side groups, wherein the sidechains, pendant groups, or side groups may be the same or different. In one embodiment one or more sidechains, pendant groups, or side groups may be covalently bonded to one or more stabilizing polymer, wherein the sidechains, pendant groups, or side groups may be the same or different.

In one embodiment one or more backbone chains of a stabilizing polymer may comprise one or more sidechains, pendant groups, or side groups, wherein the sidechains, pendant groups, or side groups may be the same or different. In one embodiment one or more sidechains, pendant groups, or side groups may be covalently bonded to one or more backbone chain of a stabilizing polymer, wherein the sidechains, pendant groups, or side groups may be the same or different.

In one embodiment one or more backbone repeat units of a stabilizing polymer may comprise one or more sidechains, pendant groups, or side groups, wherein the sidechains, pendant groups, or side groups may be the same or different. In one embodiment one or more backbone repeat units of a stabilizing polymer may be covalently bonded to one or more sidechains, pendant groups, or side groups, wherein the sidechains, pendant groups, or side groups may be the same or different.

In one embodiment a stabilizing polymer may be comprised of one or more stabilizing monomer, as described herein. In one embodiment one or more sidechains, pendant groups, or side groups of a stabilizing polymer may be comprised of one or more stabilizing monomer, as described herein. In one embodiment one or more backbone chain of a stabilizing polymer may be comprised of one or more stabilizing monomer, as described herein. In one embodiment one or more backbone repeat units of a stabilizing polymer may be comprised of one or more stabilizing monomers, as described herein. In one embodiment one or more stabilizing monomers may be covalently bonded to one or more backbone repeat units of a stabilizing polymer. In one embodiment one or more stabilizing monomer may be covalently bonded to one or more backbone chain of a stabilizing polymer. In one embodiment one or more stabilizing monomer may be covalently bonded to one or more sidechains, pendant groups, or side groups of a stabilizing polymer. In one embodiment one or more stabilizing monomer may be covalently bonded to one or more stabilizing polymer.

In one embodiment one or more stabilizing monomer may be at least part of one or more sidechains, pendant groups, or side groups of a stabilizing polymer. In one embodiment one or more stabilizing monomer may be at least part of one or more backbone repeat unit of a stabilizing polymer. In one embodiment one or more stabilizing monomer may be at least part of one or more backbone chain of a stabilizing polymer. In one embodiment one or more stabilizing monomer may be at least part of one or more stabilizing polymer.

In one embodiment a stabilizing polymer may comprise at least part of one or more stabilizing monomer. In one embodiment one or more sidechains, pendant groups, or side groups of a stabilizing polymer may comprise at least part of one or more stabilizing monomer. In one embodiment one or more backbone chain of a stabilizing polymer may comprise at least part of one or more stabilizing monomer. In one embodiment one or more backbone repeat units of a stabilizing polymer may comprise at least part of one or more stabilizing monomer.

In one embodiment an RNA stabilizing substance may comprise a polymer. In one embodiment an RNA stabilizing substance may comprise a stabilizing polymer. In one embodiment an RNA stabilizing substance may comprise an aprotic stabilizing polymer. In one embodiment an RNA stabilizing substance may comprise a polar stabilizing polymer. In one embodiment an RNA stabilizing substance may comprise a polar aprotic stabilizing polymer. In one embodiment an RNA stabilizing substance may comprise an anionic or polyanionic stabilizing polymer. In one embodiment an RNA stabilizing substance may comprise a cationic or polycationic stabilizing polymer. In one embodiment an RNA stabilizing substance may comprise a zwitterionic stabilizing polymer.

In one embodiment an RNA stabilizing polymer may comprise one or more aprotic substance. In one embodiment an RNA stabilizing polymer may comprise one or more polar substance. In one embodiment an RNA stabilizing polymer may comprise one or more polar aprotic substance. In one embodiment an RNA stabilizing polymer may comprise one or more anionic or polyanionic substance. In one embodiment an RNA stabilizing polymer may comprise one or more cationic or polycationic substance. In one embodiment an RNA stabilizing polymer may comprise one or more quaternary ammonium substance. In one embodiment an RNA stabilizing polymer may comprise one or more tertiary sulfonium substance. In one embodiment an RNA stabilizing polymer may comprise one or more quaternary phosphonium substance. In one embodiment an RNA stabilizing polymer may comprise one or more zwitterionic substance. In one embodiment an RNA stabilizing polymer may comprise one or more betaine substance.

In one embodiment a stabilizing polymer may be at least partially biodegradable. In one embodiment a stabilizing polymer may be comprised of an at least partially biodegradable backbone. In one embodiment a stabilizing polymer may be comprised of an at least partially biodegradable substance. In one embodiment a stabilizing polymer may be at least partially hydrolyzable. In one embodiment a stabilizing polymer may be comprised of an at least partially hydrolyzable backbone. In one embodiment a stabilizing polymer may be comprised of an at least partially hydrolyzable substance. In one embodiment a stabilizing polymer may comprise one or more hydrolyzable bonds. In embodiment a stabilizing monomer may comprise one or more hydrolyzable bonds.

### Polymer Backbone, and Monomers, Sidechains, Pendant Groups, or Side Groups

In one embodiment one or more backbone chain of a stabilizing polymer may be comprised of one or more at least partially biodegradable substance. In one embodiment one or more backbone chain of a stabilizing polymer may be at least partially biodegradable. In one embodiment one or more backbone chain of a stabilizing polymer may be comprised of one or more at least partially hydrolyzable substance. In one embodiment one or more backbone chain of a stabilizing polymer may be at least partially hydrolyzable. In one embodiment one or more backbone chain of a stabilizing polymer may comprise one or more hydrolyzable bonds.

In one embodiment a stabilizing polymer may comprise one or more hydrolyzable bonds. In one embodiment a stabilizing polymer may comprise two or more hydrolyzable bonds. In one embodiment a stabilizing polymer may comprise five or more hydrolyzable bonds. In one embodiment a stabilizing polymer may comprise 10 or more hydrolyzable bonds. In one embodiment a stabilizing polymer may comprise 20 or more hydrolyzable bonds. In one embodiment a stabilizing polymer may comprise 50 or more hydrolyzable bonds. In one embodiment a stabilizing polymer may comprise 100 or more hydrolyzable bonds.

In one embodiment a stabilizing polymer may comprise between about 1-1,000 hydrolyzable bonds, or between about 1-500 hydrolyzable bonds, or between about 1-100 hydrolyzable bonds, or between about 1-50 hydrolyzable bonds, or between about 1-20 hydrolyzable bonds, or between about 5-1,000 hydrolyzable bonds, or between about 5-500 hydrolyzable bonds, or between about 5-100 hydrolyzable bonds, or between about 5-50 hydrolyzable bonds, or between about 5-20 hydrolyzable bonds, or between about 10-1,000 hydrolyzable bonds, or between about 10-500 hydrolyzable bonds, or between about 10-100 hydrolyzable bonds, or between about 10-50 hydrolyzable bonds, or between about 10-20 hydrolyzable bonds, or between about 20-1,000 hydrolyzable bonds, or between about 20-500 hydrolyzable bonds, or between about 20-100 hydrolyzable bonds, or between about 20-50 hydrolyzable bonds.

In one embodiment a backbone chain of a stabilizing polymer may comprise one or more hydrolyzable bonds. In one embodiment a backbone chain of a stabilizing polymer may comprise two or more hydrolyzable bonds. In one embodiment a backbone chain of a stabilizing polymer may comprise five or more hydrolyzable bonds. In one embodiment a backbone chain of a stabilizing polymer may comprise 10 or more hydrolyzable bonds. In one embodiment a backbone chain of a stabilizing polymer may comprise 20 or more hydrolyzable bonds. In one embodiment a backbone chain of a stabilizing polymer may comprise 50 or more hydrolyzable bonds. In one embodiment a backbone chain of a stabilizing polymer may comprise 100 or more hydrolyzable bonds.

In one embodiment a backbone chain of a stabilizing polymer may comprise between about 1-1,000 hydrolyzable bonds, or between about 1-500 hydrolyzable bonds, or between about 1-100 hydrolyzable bonds, or between about 1-50 hydrolyzable bonds, or between about 1-20 hydrolyzable bonds, or between about 5-1,000 hydrolyzable bonds, or between about 5-500 hydrolyzable bonds, or between about 5-100 hydrolyzable bonds, or between about 5-50 hydrolyzable bonds, or between about 5-20 hydrolyzable bonds, or between about 10-1,000 hydrolyzable bonds, or between about 10-500 hydrolyzable bonds, or between about 10-100 hydrolyzable bonds, or between about 10-50 hydrolyzable bonds, or between about 10-20 hydrolyzable bonds, or between about 20-1,000 hydrolyzable bonds, or between about 20-500 hydrolyzable bonds, or between about 20-100 hydrolyzable bonds, or between about 20-50 hydrolyzable bonds.

In one embodiment of the present invention a stabilizing polymer may be comprised of one or more stabilizing monomer as described herein. In another embodiment a stabilizing polymer may be comprised of one or more stabilizing monomers, wherein the stabilizing monomers may be the same or different.

In one embodiment a stabilizing polymer may be comprised of one or more stabilizing monomers, wherein the stabilizing monomers may be the same or different. In one embodiment a stabilizing polymer may be comprised of two or more stabilizing monomers, wherein the stabilizing monomers may be the same or different. In one embodiment a stabilizing polymer may be comprised of five or more stabilizing monomers, wherein the stabilizing monomers may be the same or different. In one embodiment a stabilizing polymer may be comprised of 10 or more stabilizing monomers, wherein the stabilizing monomers may be the same or different. In one embodiment a stabilizing polymer may be comprised of 20 or more stabilizing monomers, wherein the stabilizing monomers may be the same or different. In one embodiment a stabilizing polymer may be comprised of 50 or more stabilizing monomers, wherein the stabilizing monomers may be the same or different. In one embodiment a stabilizing polymer may be comprised of 100 or more stabilizing monomers, wherein the stabilizing monomers may be the same or different. In one embodiment a stabilizing polymer may be comprised of 1,000 or more stabilizing monomers, wherein the stabilizing monomers may be the same or different.

In another embodiment a stabilizing polymer may be comprised of between about 2-100,000 stabilizing monomers, wherein the stabilizing monomers may be the same or different. In another embodiment a stabilizing polymer may be comprised of between about 2-10,000 stabilizing monomers, wherein the stabilizing monomers may be the same or different. In another embodiment a stabilizing polymer may be comprised of between about 2-1,000 stabilizing monomers, wherein the stabilizing monomers may be the same or different. In another embodiment a stabilizing polymer may be comprised of between about 2-500 stabilizing monomers, wherein the stabilizing monomers may be the same or different. In another embodiment a stabilizing polymer may be comprised of between about 2-250 stabilizing monomers, wherein the stabilizing monomers may be the same or different. In another embodiment a stabilizing polymer may be comprised of between about 2-100 stabilizing monomers, wherein the stabilizing monomers may be the same or different. In another embodiment a stabilizing polymer may be comprised of between about 2-50 stabilizing monomers, wherein the stabilizing monomers may be the same or different. In another embodiment a stabilizing polymer may be comprised of between about 2-20 stabilizing monomers, wherein the stabilizing monomers may be the same or different. In another embodiment a stabilizing polymer may be comprised of between about 2-10 stabilizing monomers, wherein the stabilizing monomers may be the same or different.

In another embodiment a stabilizing polymer may be comprised of between about 5-100,000 stabilizing monomers, wherein the stabilizing monomers may be the same or different. In another embodiment a stabilizing polymer may be comprised of between about 5-10,000 stabilizing monomers, wherein the stabilizing monomers may be the same or different. In another embodiment a stabilizing polymer may be comprised of between about 5-1,000 stabilizing monomers, wherein the stabilizing monomers may be the same or different. In another embodiment a stabilizing polymer may be comprised of between about 5-500 stabilizing monomers, wherein the stabilizing monomers may be the same or different. In another embodiment a stabilizing polymer may be comprised of between about 5-250 stabilizing monomers, wherein the stabilizing monomers may be the same or different. In another embodiment a stabilizing polymer may be comprised of between about 5-100 stabilizing monomers, wherein the stabilizing monomers may be the same or different. In another embodiment a stabilizing polymer may be comprised of between about 5-50 stabilizing monomers, wherein the stabilizing monomers may be the same or different. In another embodiment a stabilizing polymer may be comprised of between about 5-20 stabilizing monomers, wherein the stabilizing monomers may be the same or different. In another embodiment a stabilizing polymer may be comprised of between about 5-10 stabilizing monomers, wherein the stabilizing monomers may be the same or different.

In another embodiment a stabilizing polymer may be comprised of between about 10-100,000 stabilizing monomers, wherein the stabilizing monomers may be the same or different. In another embodiment a stabilizing polymer may be comprised of between about 10-10,000 stabilizing monomers, wherein the stabilizing monomers may be the same or different. In another embodiment a stabilizing polymer may be comprised of between about 10-1,000 stabilizing monomers, wherein the stabilizing monomers may be the same or different. In another embodiment a stabilizing polymer may be comprised of between about 10-500 stabilizing monomers, wherein the stabilizing monomers may be the same or different. In another embodiment a stabilizing polymer may be comprised of between about 10-250 stabilizing monomers, wherein the stabilizing monomers may be the same or different. In another embodiment a stabilizing polymer may be comprised of between about 10-100 stabilizing monomers, wherein the stabilizing monomers may be the same or different. In another embodiment a stabilizing polymer may be comprised of between about 10-50 stabilizing monomers, wherein the stabilizing monomers may be the same or different. In another embodiment a stabilizing polymer may be comprised of between about 10-20 stabilizing monomers, wherein the stabilizing monomers may be the same or different.

In another embodiment a stabilizing polymer may be comprised of between about 20-100,000 stabilizing monomers, wherein the stabilizing monomers may be the same or different. In another embodiment a stabilizing polymer may be comprised of between about 20-10,000 stabilizing monomers, wherein the stabilizing monomers may be the same or different. In another embodiment a stabilizing polymer may be comprised of between about 20-1,000 stabilizing monomers, wherein the stabilizing monomers may be the same or different. In another embodiment a stabilizing polymer may be comprised of between about 20-500 stabilizing monomers, wherein the stabilizing monomers may be the same or different. In another embodiment a stabilizing polymer may be comprised of between about 20-250 stabilizing monomers, wherein the stabilizing monomers may be the same or different. In another embodiment a stabilizing polymer may be comprised of between about 20-100 stabilizing monomers, wherein the stabilizing monomers may be the same or different. In another embodiment a stabilizing polymer may be comprised of between about 20-50 stabilizing monomers, wherein the stabilizing monomers may be the same or different.

In another embodiment a stabilizing polymer may be comprised of one or more sidechains, pendant groups, or side groups, wherein the sidechains, pendant groups, or side groups may be the same or different.

In one embodiment a stabilizing polymer may be comprised of one or more sidechains, pendant groups, or side groups, wherein the sidechains, pendant groups, or side groups may be the same or different. In one embodiment a stabilizing polymer may be comprised of two or more sidechains, pendant groups, or side groups, wherein the sidechains, pendant groups, or side groups may be the same or different. In one embodiment a stabilizing polymer may be comprised of five or more sidechains, pendant groups, or side groups, wherein the sidechains, pendant groups, or side groups may be the same or different. In one embodiment a stabilizing polymer may be comprised of 10 or more sidechains, pendant groups, or side groups, wherein the sidechains, pendant groups, or side groups may be the same or different. In one embodiment a stabilizing polymer may be comprised of 20 or more sidechains, pendant groups, or side groups, wherein the sidechains, pendant groups, or side groups may be the same or different. In one embodiment a stabilizing polymer may be comprised of 50 or more sidechains, pendant groups, or side groups, wherein the sidechains, pendant groups, or side groups may be the same or different. In one embodiment a stabilizing polymer may be comprised of 100 or more sidechains, pendant groups, or side groups, wherein the sidechains, pendant groups, or side groups may be the same or different. In one embodiment a stabilizing polymer may be comprised of 1,000 or more sidechains, pendant groups, or side groups, wherein the sidechains, pendant groups, or side groups may be the same or different.

In another embodiment a stabilizing polymer may be comprised of between about 2-100,000 sidechains, pendant groups, or side groups, wherein the sidechains, pendant groups, or side groups may be the same or different. In another embodiment a stabilizing polymer may be comprised of between about 2-10,000 sidechains, pendant groups, or side groups, wherein the sidechains, pendant groups, or side groups may be the same or different. In another embodiment a stabilizing polymer may be comprised of between about 2-1,000 sidechains, pendant groups, or side groups, wherein the sidechains, pendant groups, or side groups may be the same or different. In another embodiment a stabilizing polymer may be comprised of between about 2-500 sidechains, pendant groups, or side groups, wherein the sidechains, pendant groups, or side groups may be the same or different. In another embodiment a stabilizing polymer may be comprised of between about 2-250 sidechains, pendant groups, or side groups, wherein the sidechains, pendant groups, or side groups may be the same or different. In another embodiment a stabilizing polymer may be comprised of between about 2-100 sidechains, pendant groups, or side groups, wherein the sidechains, pendant groups, or side groups may be the same or different. In another embodiment a stabilizing polymer may be comprised of between about 2-50 sidechains, pendant groups, or side groups, wherein the sidechains, pendant groups, or side groups may be the same or different. In another embodiment a stabilizing polymer may be comprised of between about 2-20 sidechains, pendant groups, or side groups, wherein the sidechains, pendant groups, or side groups may be the same or different. In another embodiment a stabilizing polymer may be comprised of between about 2-10 sidechains, pendant groups, or side groups, wherein the sidechains, pendant groups, or side groups may be the same or different.

In another embodiment a stabilizing polymer may be comprised of between about 5-100,000 sidechains, pendant groups, or side groups, wherein the sidechains, pendant groups, or side groups may be the same or different. In another embodiment a stabilizing polymer may be comprised of between about 5-10,000 sidechains, pendant groups, or side groups, wherein the sidechains, pendant groups, or side groups may be the same or different. In another embodiment a stabilizing polymer may be comprised of between about 5-1,000 sidechains, pendant groups, or side groups, wherein the sidechains, pendant groups, or side groups may be the same or different. In another embodiment a stabilizing polymer may be comprised of between about 5-500 sidechains, pendant groups, or side groups, wherein the sidechains, pendant groups, or side groups may be the same or different. In another embodiment a stabilizing polymer may be comprised of between about 5-250 sidechains, pendant groups, or side groups, wherein the sidechains, pendant groups, or side groups may be the same or different. In another embodiment a stabilizing polymer may be comprised of between about 5-100 sidechains, pendant groups, or side groups, wherein the sidechains, pendant groups, or side groups may be the same or different. In another embodiment a stabilizing polymer may be comprised of between about 5-50 sidechains, pendant groups, or side groups, wherein the sidechains, pendant groups, or side groups may be the same or different. In another embodiment a stabilizing polymer may be comprised of between about 5-20 sidechains, pendant groups, or side groups, wherein the sidechains, pendant groups, or side groups may be the same or different. In another embodiment a stabilizing polymer may be comprised of between about 5-10 sidechains, pendant groups, or side groups, wherein the sidechains, pendant groups, or side groups may be the same or different.

In another embodiment a stabilizing polymer may be comprised of between about 10-100,000 sidechains, pendant groups, or side groups, wherein the sidechains, pendant groups, or side groups may be the same or different. In another embodiment a stabilizing polymer may be comprised of between about 10-10,000 sidechains, pendant groups, or side groups, wherein the sidechains, pendant groups, or side groups may be the same or different. In another embodiment a stabilizing polymer may be comprised of between about 10-1,000 sidechains, pendant groups, or side groups, wherein the sidechains, pendant groups, or side groups may be the same or different. In another embodiment a stabilizing polymer may be comprised of between about 10-500 sidechains, pendant groups, or side groups, wherein the sidechains, pendant groups, or side groups may be the same or different. In another embodiment a stabilizing polymer may be comprised of between about 10-250 sidechains, pendant groups, or side groups, wherein the sidechains, pendant groups, or side groups may be the same or different. In another embodiment a stabilizing polymer may be comprised of between about 10-100 sidechains, pendant groups, or side groups, wherein the sidechains, pendant groups, or side groups may be the same or different. In another embodiment a stabilizing polymer may be comprised of between about 10-50 sidechains, pendant groups, or side groups, wherein the sidechains, pendant groups, or side groups may be the same or different. In another embodiment a stabilizing polymer may be comprised of between about 10-sidechains, pendant groups, or side groups, wherein the sidechains, pendant groups, or side groups may be the same or different.

In another embodiment a stabilizing polymer may be comprised of between about 20-100,000 sidechains, pendant groups, or side groups, wherein the sidechains, pendant groups, or side groups may be the same or different. In another embodiment a stabilizing polymer may be comprised of between about 20-10,000 sidechains, pendant groups, or side groups, wherein the sidechains, pendant groups, or side groups may be the same or different. In another embodiment a stabilizing polymer may be comprised of between about 20-1,000 sidechains, pendant groups, or side groups, wherein the sidechains, pendant groups, or side groups may be the same or different. In another embodiment a stabilizing polymer may be comprised of between about 20-500 sidechains, pendant groups, or side groups, wherein the sidechains, pendant groups, or side groups may be the same or different. In another embodiment a stabilizing polymer may be comprised of between about 20-250 sidechains, pendant groups, or side groups, wherein the sidechains, pendant groups, or side groups may be the same or different. In another embodiment a stabilizing polymer may be comprised of between about 20-100 sidechains, pendant groups, or side groups, wherein the sidechains, pendant groups, or side groups may be the same or different. In another embodiment a stabilizing polymer may be comprised of between about 20-50 sidechains, pendant groups, or side groups, wherein the sidechains, pendant groups, or side groups may be the same or different.

In one embodiment a backbone chain of a stabilizing polymer may be comprised of one or more stabilizing monomers, wherein the stabilizing monomers may be the same or different. In one embodiment a backbone chain of a stabilizing polymer may be comprised of two or more stabilizing monomers, wherein the stabilizing monomers may be the same or different. In one embodiment a backbone chain of a stabilizing polymer may be comprised of five or more stabilizing monomers, wherein the stabilizing monomers may be the same or different. In one embodiment a backbone chain of a stabilizing polymer may be comprised of 10 or more stabilizing monomers, wherein the stabilizing monomers may be the same or different. In one embodiment a backbone chain of a stabilizing polymer may be comprised of 20 or more stabilizing monomers, wherein the stabilizing monomers may be the same or different. In one embodiment a backbone chain of a stabilizing polymer may be comprised of 50 or more stabilizing monomers, wherein the stabilizing monomers may be the same or different. In one embodiment a backbone chain of a stabilizing polymer may be comprised of 100 or more stabilizing monomers, wherein the stabilizing monomers may be the same or different. In one embodiment a backbone chain of a stabilizing polymer may be comprised of 1,000 or more stabilizing monomers, wherein the stabilizing monomers may be the same or different.

In another embodiment a backbone chain of a stabilizing polymer may be comprised of between about 2-100,000 stabilizing monomers, wherein the stabilizing monomers may be the same or different. In another embodiment a backbone chain of a stabilizing polymer may be comprised of between about 2-10,000 stabilizing monomers, wherein the stabilizing monomers may be the same or different. In another embodiment a backbone chain of a stabilizing polymer may be comprised of between about 2-1,000 stabilizing monomers, wherein the stabilizing monomers may be the same or different. In another embodiment a backbone chain of a stabilizing polymer may be comprised of between about 2-500 stabilizing monomers, wherein the stabilizing monomers may be the same or different. In another embodiment a backbone chain of a stabilizing polymer may be comprised of between about 2-250 stabilizing monomers, wherein the stabilizing monomers may be the same or different. In another embodiment a backbone chain of a stabilizing polymer may be comprised of between about 2-100 stabilizing monomers, wherein the stabilizing monomers may be the same or different. In another embodiment a backbone chain of a stabilizing polymer may be comprised of between about 2-50 stabilizing monomers, wherein the stabilizing monomers may be the same or different. In another embodiment a backbone chain of a stabilizing polymer may be comprised of between about 2-20 stabilizing monomers, wherein the stabilizing monomers may be the same or different. In another embodiment a backbone chain of a stabilizing polymer may be comprised of between about 2-10 stabilizing monomers, wherein the stabilizing monomers may be the same or different.

In another embodiment a backbone chain of a stabilizing polymer may be comprised of between about 5-100,000 stabilizing monomers, wherein the stabilizing monomers may be the same or different. In another embodiment a backbone chain of a stabilizing polymer may be comprised of between about 5-10,000 stabilizing monomers, wherein the stabilizing monomers may be the same or different. In another embodiment a backbone chain of a stabilizing polymer may be comprised of between about 5-1,000 stabilizing monomers, wherein the stabilizing monomers may be the same or different. In another embodiment a backbone chain of a stabilizing polymer may be comprised of between about 5-500 stabilizing monomers, wherein the stabilizing monomers may be the same or different. In another embodiment a backbone chain of a stabilizing polymer may be comprised of between about 5-250 stabilizing monomers, wherein the stabilizing monomers may be the same or different. In another embodiment a backbone chain of a stabilizing polymer may be comprised of between about 5-100 stabilizing monomers, wherein the stabilizing monomers may be the same or different. In another embodiment a backbone chain of a stabilizing polymer may be comprised of between about 5-50 stabilizing monomers, wherein the stabilizing monomers may be the same or different. In another embodiment a backbone chain of a stabilizing polymer may be comprised of between about 5-20 stabilizing monomers, wherein the stabilizing monomers may be the same or different. In another embodiment a backbone chain of a stabilizing polymer may be comprised of between about 5-10 stabilizing monomers, wherein the stabilizing monomers may be the same or different.

In another embodiment a backbone chain of a stabilizing polymer may be comprised of between about 10-100,000 stabilizing monomers, wherein the stabilizing monomers may be the same or different. In another embodiment a backbone chain of a stabilizing polymer may be comprised of between about 10-10,000 stabilizing monomers, wherein the stabilizing monomers may be the same or different. In another embodiment a backbone chain of a stabilizing polymer may be comprised of between about 10-1,000 stabilizing monomers, wherein the stabilizing monomers may be the same or different. In another embodiment a backbone chain of a stabilizing polymer may be comprised of between about 10-500 stabilizing monomers, wherein the stabilizing monomers may be the same or different. In another embodiment a backbone chain of a stabilizing polymer may be comprised of between about 10-250 stabilizing monomers, wherein the stabilizing monomers may be the same or different. In another embodiment a backbone chain of a stabilizing polymer may be comprised of between about 10-100 stabilizing monomers, wherein the stabilizing monomers may be the same or different. In another embodiment a backbone chain of a stabilizing polymer may be comprised of between about 10-50 stabilizing monomers, wherein the stabilizing monomers may be the same or different. In another embodiment a backbone chain of a stabilizing polymer may be comprised of between about 10-20 stabilizing monomers, wherein the stabilizing monomers may be the same or different.

In another embodiment a backbone chain of a stabilizing polymer may be comprised of between about 20-100,000 stabilizing monomers, wherein the stabilizing monomers may be the same or different. In another embodiment a backbone chain of a stabilizing polymer may be comprised of between about 20-10,000 stabilizing monomers, wherein the stabilizing monomers may be the same or different. In another embodiment a backbone chain of a stabilizing polymer may be comprised of between about 20-1,000 stabilizing monomers, wherein the stabilizing monomers may be the same or different. In another embodiment a backbone chain of a stabilizing polymer may be comprised of between about 20-500 stabilizing monomers, wherein the stabilizing monomers may be the same or different. In another embodiment a backbone chain of a stabilizing polymer may be comprised of between about 20-250 stabilizing monomers, wherein the stabilizing monomers may be the same or different. In another embodiment a backbone chain of a stabilizing polymer may be comprised of between about 20-100 stabilizing monomers, wherein the stabilizing monomers may be the same or different. In another embodiment a backbone chain of a stabilizing polymer may be comprised of between about 20-50 stabilizing monomers, wherein the stabilizing monomers may be the same or different.

In another embodiment a backbone chain of a stabilizing polymer may be comprised of one or more sidechains, pendant groups, or side groups, wherein the sidechains, pendant groups, or side groups may be the same or different.

In one embodiment a backbone chain of a stabilizing polymer may be comprised of one or more sidechains, pendant groups, or side groups, wherein the sidechains, pendant groups, or side groups may be the same or different. In one embodiment a backbone chain of a stabilizing polymer may be comprised of two or more sidechains, pendant groups, or side groups, wherein the sidechains, pendant groups, or side groups may be the same or different. In one embodiment a backbone chain of a stabilizing polymer may be comprised of five or more sidechains, pendant groups, or side groups, wherein the sidechains, pendant groups, or side groups may be the same or different. In one embodiment a backbone chain of a stabilizing polymer may be comprised of 10 or more sidechains, pendant groups, or side groups, wherein the sidechains, pendant groups, or side groups may be the same or different. In one embodiment a backbone chain of a stabilizing polymer may be comprised of 20 or more sidechains, pendant groups, or side groups, wherein the sidechains, pendant groups, or side groups may be the same or different. In one embodiment a backbone chain of a stabilizing polymer may be comprised of 50 or more sidechains, pendant groups, or side groups, wherein the sidechains, pendant groups, or side groups may be the same or different. In one embodiment a backbone chain of a stabilizing polymer may be comprised of 100 or more sidechains, pendant groups, or side groups, wherein the sidechains, pendant groups, or side groups may be the same or different. In one embodiment a backbone chain of a stabilizing polymer may be comprised of 1,000 or more sidechains, pendant groups, or side groups, wherein the sidechains, pendant groups, or side groups may be the same or different.

In another embodiment a backbone chain of a stabilizing polymer may be comprised of between about 2-100,000 sidechains, pendant groups, or side groups, wherein the sidechains, pendant groups, or side groups may be the same or different. In another embodiment a backbone chain of a stabilizing polymer may be comprised of between about 2-10,000 sidechains, pendant groups, or side groups, wherein the sidechains, pendant groups, or side groups may be the same or different. In another embodiment a backbone chain of a stabilizing polymer may be comprised of between about 2-1,000 sidechains, pendant groups, or side groups, wherein the sidechains, pendant groups, or side groups may be the same or different. In another embodiment a backbone chain of a stabilizing polymer may be comprised of between about 2-500 sidechains, pendant groups, or side groups, wherein the sidechains, pendant groups, or side groups may be the same or different. In another embodiment a backbone chain of a stabilizing polymer may be comprised of between about 2-250 sidechains, pendant groups, or side groups, wherein the sidechains, pendant groups, or side groups may be the same or different. In another embodiment a backbone chain of a stabilizing polymer may be comprised of between about 2-100 sidechains, pendant groups, or side groups, wherein the sidechains, pendant groups, or side groups may be the same or different. In another embodiment a backbone chain of a stabilizing polymer may be comprised of between about 2-50 sidechains, pendant groups, or side groups, wherein the sidechains, pendant groups, or side groups may be the same or different. In another embodiment a backbone chain of a stabilizing polymer may be comprised of between about 2-20 sidechains, pendant groups, or side groups, wherein the sidechains, pendant groups, or side groups may be the same or different. In another embodiment a backbone chain of a stabilizing polymer may be comprised of between about 2-10 sidechains, pendant groups, or side groups, wherein the sidechains, pendant groups, or side groups may be the same or different.

In another embodiment a backbone chain of a stabilizing polymer may be comprised of between about 5-100,000 sidechains, pendant groups, or side groups, wherein the sidechains, pendant groups, or side groups may be the same or different. In another embodiment a backbone chain of a stabilizing polymer may be comprised of between about 5-10,000 sidechains, pendant groups, or side groups, wherein the sidechains, pendant groups, or side groups may be the same or different. In another embodiment a backbone chain of a stabilizing polymer may be comprised of between about 5-1,000 sidechains, pendant groups, or side groups, wherein the sidechains, pendant groups, or side groups may be the same or different. In another embodiment a backbone chain of a stabilizing polymer may be comprised of between about 5-500 sidechains, pendant groups, or side groups, wherein the sidechains, pendant groups, or side groups may be the same or different. In another embodiment a backbone chain of a stabilizing polymer may be comprised of between about 5-250 sidechains, pendant groups, or side groups, wherein the sidechains, pendant groups, or side groups may be the same or different. In another embodiment a backbone chain of a stabilizing polymer may be comprised of between about 5-100 sidechains, pendant groups, or side groups, wherein the sidechains, pendant groups, or side groups may be the same or different. In another embodiment a backbone chain of a stabilizing polymer may be comprised of between about 5-50 sidechains, pendant groups, or side groups, wherein the sidechains, pendant groups, or side groups may be the same or different. In another embodiment a backbone chain of a stabilizing polymer may be comprised of between about 5-20 sidechains, pendant groups, or side groups, wherein the sidechains, pendant groups, or side groups may be the same or different. In another embodiment a backbone chain of a stabilizing polymer may be comprised of between about 5-10 sidechains, pendant groups, or side groups, wherein the sidechains, pendant groups, or side groups may be the same or different.

In another embodiment a backbone chain of a stabilizing polymer may be comprised of between about 10-100,000 sidechains, pendant groups, or side groups, wherein the sidechains, pendant groups, or side groups may be the same or different. In another embodiment a backbone chain of a stabilizing polymer may be comprised of between about 10-10,000 sidechains, pendant groups, or side groups, wherein the sidechains, pendant groups, or side groups may be the same or different. In another embodiment a backbone chain of a stabilizing polymer may be comprised of between about 10-1,000 sidechains, pendant groups, or side groups, wherein the sidechains, pendant groups, or side groups may be the same or different. In another embodiment a backbone chain of a stabilizing polymer may be comprised of between about 10-500 sidechains, pendant groups, or side groups, wherein the sidechains, pendant groups, or side groups may be the same or different. In another embodiment a backbone chain of a stabilizing polymer may be comprised of between about 10-250 sidechains, pendant groups, or side groups, wherein the sidechains, pendant groups, or side groups may be the same or different. In another embodiment a backbone chain of a stabilizing polymer may be comprised of between about 10-100 sidechains, pendant groups, or side groups, wherein the sidechains, pendant groups, or side groups may be the same or different. In another embodiment a backbone chain of a stabilizing polymer may be comprised of between about 10-50 sidechains, pendant groups, or side groups, wherein the sidechains, pendant groups, or side groups may be the same or different. In another embodiment a backbone chain of a stabilizing polymer may be comprised of between about 10-20 sidechains, pendant groups, or side groups, wherein the sidechains, pendant groups, or side groups may be the same or different.

In another embodiment a backbone chain of a stabilizing polymer may be comprised of between about 20-100,000 sidechains, pendant groups, or side groups, wherein the sidechains, pendant groups, or side groups may be the same or different. In another embodiment a backbone chain of a stabilizing polymer may be comprised of between about 20-10,000 sidechains, pendant groups, or side groups, wherein the sidechains, pendant groups, or side groups may be the same or different. In another embodiment a backbone chain of a stabilizing polymer may be comprised of between about 20-1,000 sidechains, pendant groups, or side groups, wherein the sidechains, pendant groups, or side groups may be the same or different. In another embodiment a backbone chain of a stabilizing polymer may be comprised of between about 20-500 sidechains, pendant groups, or side groups, wherein the sidechains, pendant groups, or side groups may be the same or different. In another embodiment a backbone chain of a stabilizing polymer may be comprised of between about 20-250 sidechains, pendant groups, or side groups, wherein the sidechains, pendant groups, or side groups may be the same or different. In another embodiment a backbone chain of a stabilizing polymer may be comprised of between about 20-100 sidechains, pendant groups, or side groups, wherein the sidechains, pendant groups, or side groups may be the same or different. In another embodiment a backbone chain of a stabilizing polymer may be comprised of between about 20-50 sidechains, pendant groups, or side groups, wherein the sidechains, pendant groups, or side groups may be the same or different.

In one embodiment a stabilizing polymer may be comprised of one or more backbone repeat units, wherein the backbone repeat units may be the same or different. In one embodiment a stabilizing polymer may be comprised of two or more backbone repeat units, wherein the backbone repeat units may be the same or different. In one embodiment a stabilizing polymer may be comprised of five or more backbone repeat units, wherein the backbone repeat units may be the same or different. In one embodiment a stabilizing polymer may be comprised of 10 or more backbone repeat units, wherein the backbone repeat units may be the same or different. In one embodiment a stabilizing polymer may be comprised of 20 or more backbone repeat units, wherein the backbone repeat units may be the same or different. In one embodiment a stabilizing polymer may be comprised of 50 or more backbone repeat units, wherein the backbone repeat units may be the same or different. In one embodiment a stabilizing polymer may be comprised of 100 or more backbone repeat units, wherein the backbone repeat units may be the same or different. In one embodiment a stabilizing polymer may be comprised of 1,000 or more backbone repeat units, wherein the backbone repeat units may be the same or different.

In another embodiment a stabilizing polymer may be comprised of between about 2-100,000 backbone repeat units, wherein the backbone repeat units may be the same or different. In another embodiment a stabilizing polymer may be comprised of between about 2-10,000 backbone repeat units, wherein the backbone repeat units may be the same or different. In another embodiment a stabilizing polymer may be comprised of between about 2-1,000 backbone repeat units, wherein the backbone repeat units may be the same or different. In another embodiment a stabilizing polymer may be comprised of between about 2-500 backbone repeat units, wherein the backbone repeat units may be the same or different. In another embodiment a stabilizing polymer may be comprised of between about 2-400 backbone repeat units, wherein the backbone repeat units may be the same or different. In another embodiment a stabilizing polymer may be comprised of between about 2-300 backbone repeat units, wherein the backbone repeat units may be the same or different. In another embodiment a stabilizing polymer may be comprised of between about 2-250 backbone repeat units, wherein the backbone repeat units may be the same or different. In another embodiment a stabilizing polymer may be comprised of between about 2-200 backbone repeat units, wherein the backbone repeat units may be the same or different. In another embodiment a stabilizing polymer may be comprised of between about 2-150 backbone repeat units, wherein the backbone repeat units may be the same or different. In another embodiment a stabilizing polymer may be comprised of between about 2-100 backbone repeat units, wherein the backbone repeat units may be the same or different. In another embodiment a stabilizing polymer may be comprised of between about 2-50 backbone repeat units, wherein the backbone repeat units may be the same or different. In another embodiment a stabilizing polymer may be comprised of between about 2-20 backbone repeat units, wherein the backbone repeat units may be the same or different.

In another embodiment a stabilizing polymer may be comprised of between about 5-100,000 backbone repeat units, wherein the backbone repeat units may be the same or different. In another embodiment a stabilizing polymer may be comprised of between about 5-10,000 backbone repeat units, wherein the backbone repeat units may be the same or different. In another embodiment a stabilizing polymer may be comprised of between about 5-1,000 backbone repeat units, wherein the backbone repeat units may be the same or different. In another embodiment a stabilizing polymer may be comprised of between about 5-500 backbone repeat units, wherein the backbone repeat units may be the same or different. In another embodiment a stabilizing polymer may be comprised of between about 5-400 backbone repeat units, wherein the backbone repeat units may be the same or different. In another embodiment a stabilizing polymer may be comprised of between about 5-300 backbone repeat units, wherein the backbone repeat units may be the same or different. In another embodiment a stabilizing polymer may be comprised of between about 5-250 backbone repeat units, wherein the backbone repeat units may be the same or different. In another embodiment a stabilizing polymer may be comprised of between about 5-200 backbone repeat units, wherein the backbone repeat units may be the same or different. In another embodiment a stabilizing polymer may be comprised of between about 5-150 backbone repeat units, wherein the backbone repeat units may be the same or different. In another embodiment a stabilizing polymer may be comprised of between about 5-100 backbone repeat units, wherein the backbone repeat units may be the same or different. In another embodiment a stabilizing polymer may be comprised of between about 5-50 backbone repeat units, wherein the backbone repeat units may be the same or different. In another embodiment a stabilizing polymer may be comprised of between about 5-20 backbone repeat units, wherein the backbone repeat units may be the same or different.

In another embodiment a stabilizing polymer may be comprised of between about 10-100,000 backbone repeat units, wherein the backbone repeat units may be the same or different. In another embodiment a stabilizing polymer may be comprised of between about 10-10,000 backbone repeat units, wherein the backbone repeat units may be the same or different. In another embodiment a stabilizing polymer may be comprised of between about 10-1,000 backbone repeat units, wherein the backbone repeat units may be the same or different. In another embodiment a stabilizing polymer may be comprised of between about 10-500 backbone repeat units, wherein the backbone repeat units may be the same or different. In another embodiment a stabilizing polymer may be comprised of between about 10-400 backbone repeat units, wherein the backbone repeat units may be the same or different. In another embodiment a stabilizing polymer may be comprised of between about 10-300 backbone repeat units, wherein the backbone repeat units may be the same or different. In another embodiment a stabilizing polymer may be comprised of between about 10-250 backbone repeat units, wherein the backbone repeat units may be the same or different. In another embodiment a stabilizing polymer may be comprised of between about 10-200 backbone repeat units, wherein the backbone repeat units may be the same or different. In another embodiment a stabilizing polymer may be comprised of between about 10-150 backbone repeat units, wherein the backbone repeat units may be the same or different. In another embodiment a stabilizing polymer may be comprised of between about 10-100 backbone repeat units, wherein the backbone repeat units may be the same or different. In another embodiment a stabilizing polymer may be comprised of between about 10-50 backbone repeat units, wherein the backbone repeat units may be the same or different. In another embodiment a stabilizing polymer may be comprised of between about 10-20 backbone repeat units, wherein the backbone repeat units may be the same or different.

In one embodiment a backbone chain of a stabilizing polymer may be comprised of one or more backbone repeat units, wherein the backbone repeat units may be the same or different. In one embodiment a backbone chain of a stabilizing polymer may be comprised of two or more backbone repeat units, wherein the backbone repeat units may be the same or different. In one embodiment a backbone chain of a stabilizing polymer may be comprised of five or more backbone repeat units, wherein the backbone repeat units may be the same or different. In one embodiment a backbone chain of a stabilizing polymer may be comprised of 10 or more backbone repeat units, wherein the backbone repeat units may be the same or different. In one embodiment a backbone chain of a stabilizing polymer may be comprised of 20 or more backbone repeat units, wherein the backbone repeat units may be the same or different. In one embodiment a backbone chain of a stabilizing polymer may be comprised of 50 or more backbone repeat units, wherein the backbone repeat units may be the same or different. In one embodiment a backbone chain of a stabilizing polymer may be comprised of 100 or more backbone repeat units, wherein the backbone repeat units may be the same or different. In one embodiment a backbone chain of a stabilizing polymer may be comprised of 1,000 or more backbone repeat units, wherein the backbone repeat units may be the same or different.

In another embodiment a backbone chain of a stabilizing polymer may be comprised of between about 2-100,000 backbone repeat units, wherein the backbone repeat units may be the same or different. In another embodiment a backbone chain of a stabilizing polymer may be comprised of between about 2-10,000 backbone repeat units, wherein the backbone repeat units may be the same or different. In another embodiment a backbone chain of a stabilizing polymer may be comprised of between about 2-1,000 backbone repeat units, wherein the backbone repeat units may be the same or different. In another embodiment a backbone chain of a stabilizing polymer may be comprised of between about 2-500 backbone repeat units, wherein the backbone repeat units may be the same or different. In another embodiment a backbone chain of a stabilizing polymer may be comprised of between about 2-400 backbone repeat units, wherein the backbone repeat units may be the same or different. In another embodiment a backbone chain of a stabilizing polymer may be comprised of between about 2-300 backbone repeat units, wherein the backbone repeat units may be the same or different. In another embodiment a backbone chain of a stabilizing polymer may be comprised of between about 2-250 backbone repeat units, wherein the backbone repeat units may be the same or different. In another embodiment a backbone chain of a stabilizing polymer may be comprised of between about 2-200 backbone repeat units, wherein the backbone repeat units may be the same or different. In another embodiment a backbone chain of a stabilizing polymer may be comprised of between about 2-150 backbone repeat units, wherein the backbone repeat units may be the same or different. In another embodiment a backbone chain of a stabilizing polymer may be comprised of between about 2-100 backbone repeat units, wherein the backbone repeat units may be the same or different. In another embodiment a backbone chain of a stabilizing polymer may be comprised of between about 2-50 backbone repeat units, wherein the backbone repeat units may be the same or different. In another embodiment a backbone chain of a stabilizing polymer may be comprised of between about 2-20 backbone repeat units, wherein the backbone repeat units may be the same or different. In another embodiment a backbone chain of a stabilizing polymer may be comprised of between about 2-10 backbone repeat units, wherein the backbone repeat units may be the same or different. In another embodiment a backbone chain of a stabilizing polymer may be comprised of between about 2-5 backbone repeat units, wherein the backbone repeat units may be the same or different.

In another embodiment a backbone chain of a stabilizing polymer may be comprised of between about 5-100,000 backbone repeat units, wherein the backbone repeat units may be the same or different. In another embodiment a backbone chain of a stabilizing polymer may be comprised of between about 5-10,000 backbone repeat units, wherein the backbone repeat units may be the same or different. In another embodiment a backbone chain of a stabilizing polymer may be comprised of between about 5-1,000 backbone repeat units, wherein the backbone repeat units may be the same or different. In another embodiment a backbone chain of a stabilizing polymer may be comprised of between about 5-500 backbone repeat units, wherein the backbone repeat units may be the same or different. In another embodiment a backbone chain of a stabilizing polymer may be comprised of between about 5-400 backbone repeat units, wherein the backbone repeat units may be the same or different. In another embodiment a backbone chain of a stabilizing polymer may be comprised of between about 5-300 backbone repeat units, wherein the backbone repeat units may be the same or different. In another embodiment a backbone chain of a stabilizing polymer may be comprised of between about 5-250 backbone repeat units, wherein the backbone repeat units may be the same or different. In another embodiment a backbone chain of a stabilizing polymer may be comprised of between about 5-200 backbone repeat units, wherein the backbone repeat units may be the same or different. In another embodiment a backbone chain of a stabilizing polymer may be comprised of between about 5-150 backbone repeat units, wherein the backbone repeat units may be the same or different. In another embodiment a backbone chain of a stabilizing polymer may be comprised of between about 5-100 backbone repeat units, wherein the backbone repeat units may be the same or different. In another embodiment a backbone chain of a stabilizing polymer may be comprised of between about 5-50 backbone repeat units, wherein the backbone repeat units may be the same or different. In another embodiment a backbone chain of a stabilizing polymer may be comprised of between about 5-20 backbone repeat units, wherein the backbone repeat units may be the same or different. In another embodiment a backbone chain of a stabilizing polymer may be comprised of between about 5-10 backbone repeat units, wherein the backbone repeat units may be the same or different.

In another embodiment a backbone chain of a stabilizing polymer may be comprised of between about 10-100,000 backbone repeat units, wherein the backbone repeat units may be the same or different. In another embodiment a backbone chain of a stabilizing polymer may be comprised of between about 10-10,000 backbone repeat units, wherein the backbone repeat units may be the same or different. In another embodiment a backbone chain of a stabilizing polymer may be comprised of between about 10-1,000 backbone repeat units, wherein the backbone repeat units may be the same or different. In another embodiment a backbone chain of a stabilizing polymer may be comprised of between about 10-500 backbone repeat units, wherein the backbone repeat units may be the same or different. In another embodiment a backbone chain of a stabilizing polymer may be comprised of between about 10-400 backbone repeat units, wherein the backbone repeat units may be the same or different. In another embodiment a backbone chain of a stabilizing polymer may be comprised of between about 10-300 backbone repeat units, wherein the backbone repeat units may be the same or different. In another embodiment a backbone chain of a stabilizing polymer may be comprised of between about 10-250 backbone repeat units, wherein the backbone repeat units may be the same or different. In another embodiment a backbone chain of a stabilizing polymer may be comprised of between about 10-200 backbone repeat units, wherein the backbone repeat units may be the same or different. In another embodiment a backbone chain of a stabilizing polymer may be comprised of between about 10-150 backbone repeat units, wherein the backbone repeat units may be the same or different. In another embodiment a backbone chain of a stabilizing polymer may be comprised of between about 10-100 backbone repeat units, wherein the backbone repeat units may be the same or different. In another embodiment a backbone chain of a stabilizing polymer may be comprised of between about 10-50 backbone repeat units, wherein the backbone repeat units may be the same or different. In another embodiment a backbone chain of a stabilizing polymer may be comprised of between about 10-20 backbone repeat units, wherein the backbone repeat units may be the same or different.

In one embodiment a stabilizing polymer may be comprised of one or more backbone chains, wherein the backbone chains may be the same or different. In one embodiment a stabilizing polymer may be comprised of two or more backbone chains, wherein the backbone chains may be the same or different. In one embodiment a stabilizing polymer may be comprised of three or more backbone chains, wherein the backbone chains may be the same or different. In one embodiment a stabilizing polymer may be comprised of four or more backbone chains, wherein the backbone chains may be the same or different. In one embodiment a stabilizing polymer may be comprised of five or more backbone chains, wherein the backbone chains may be the same or different. In one embodiment a stabilizing polymer may be comprised of 10 or more backbone chains, wherein the backbone chains may be the same or different. In one embodiment a stabilizing polymer may be comprised of 20 or more backbone chains, wherein the backbone chains may be the same or different. In one embodiment a stabilizing polymer may be comprised of 50 or more backbone chains, wherein the backbone chains may be the same or different. In one embodiment a stabilizing polymer may be comprised of 100 or more backbone chains, wherein the backbone chains may be the same or different.

In another embodiment a stabilizing polymer may be comprised of between about 2-10,000 backbone chains, wherein the backbone chains may be the same or different. In another embodiment a stabilizing polymer may be comprised of between about 2-1,000 backbone chains, wherein the backbone chains may be the same or different. In another embodiment a stabilizing polymer may be comprised of between about 2-500 backbone chains, wherein the backbone chains may be the same or different. In another embodiment a stabilizing polymer may be comprised of between about 2-250 backbone chains, wherein the backbone chains may be the same or different. In another embodiment a stabilizing polymer may be comprised of between about 2-100 backbone chains, wherein the backbone chains may be the same or different. In another embodiment a stabilizing polymer may be comprised of between about 2-50 backbone chains, wherein the backbone chains may be the same or different. In another embodiment a stabilizing polymer may be comprised of between about 2-20 backbone chains, wherein the backbone chains may be the same or different. In another embodiment a stabilizing polymer may be comprised of between about 2-10 backbone chains, wherein the backbone chains may be the same or different. In another embodiment a stabilizing polymer may be comprised of between about 2-5 backbone chains, wherein the backbone chains may be the same or different.

In another embodiment a stabilizing polymer may be comprised of between about 5-10,000 backbone chains, wherein the backbone chains may be the same or different. In another embodiment a stabilizing polymer may be comprised of between about 5-1,000 backbone chains, wherein the backbone chains may be the same or different. In another embodiment a stabilizing polymer may be comprised of between about 5-500 backbone chains, wherein the backbone chains may be the same or different. In another embodiment a stabilizing polymer may be comprised of between about 5-250 backbone chains, wherein the backbone chains may be the same or different. In another embodiment a stabilizing polymer may be comprised of between about 5-100 backbone chains, wherein the backbone chains may be the same or different. In another embodiment a stabilizing polymer may be comprised of between about 5-50 backbone chains, wherein the backbone chains may be the same or different. In another embodiment a stabilizing polymer may be comprised of between about 5-20 backbone chains, wherein the backbone chains may be the same or different. In another embodiment a stabilizing polymer may be comprised of between about 5-10 backbone chains, wherein the backbone chains may be the same or different.

In another embodiment a stabilizing polymer may be comprised of between about 10-10,000 backbone chains, wherein the backbone chains may be the same or different. In another embodiment a stabilizing polymer may be comprised of between about 10-1,000 backbone chains, wherein the backbone chains may be the same or different. In another embodiment a stabilizing polymer may be comprised of between about 10-500 backbone chains, wherein the backbone chains may be the same or different. In another embodiment a stabilizing polymer may be comprised of between about 10-250 backbone chains, wherein the backbone chains may be the same or different. In another embodiment a stabilizing polymer may be comprised of between about 10-100 backbone chains, wherein the backbone chains may be the same or different. In another embodiment a stabilizing polymer may be comprised of between about 10-50 backbone chains, wherein the backbone chains may be the same or different. In another embodiment a stabilizing polymer may be comprised of between about 10-20 backbone chains, wherein the backbone chains may be the same or different.

In one embodiment a stabilizing polymer comprised of a block copolymer may be comprised of one or more blocks, wherein the blocks may be the same or different. In one embodiment a stabilizing polymer comprised of a block copolymer may be comprised of two or more blocks, wherein the blocks may be the same or different. In one embodiment a stabilizing polymer comprised of a block copolymer may be comprised of three or more blocks, wherein the blocks may be the same or different. In one embodiment a stabilizing polymer comprised of a block copolymer may be comprised of four or more blocks, wherein the blocks may be the same or different. In one embodiment a stabilizing polymer comprised of a block copolymer may be comprised of five or more blocks, wherein the blocks may be the same or different. In one embodiment a stabilizing polymer comprised of a block copolymer may be comprised of 10 or more blocks, wherein the blocks may be the same or different. In one embodiment a stabilizing polymer comprised of a block copolymer may be comprised of 20 or more blocks, wherein the blocks may be the same or different. In one embodiment a stabilizing polymer comprised of a block copolymer may be comprised of 50 or more blocks, wherein the blocks may be the same or different. In one embodiment a stabilizing polymer comprised of a block copolymer may be comprised of 100 or more blocks, wherein the blocks may be the same or different.

In another embodiment a stabilizing polymer comprised of a block copolymer may be comprised of between about 2-10,000 blocks, wherein the blocks may be the same or different. In another embodiment a stabilizing polymer comprised of a block copolymer may be comprised of between about 2-1,000 blocks, wherein the blocks may be the same or different. In another embodiment a stabilizing polymer comprised of a block copolymer may be comprised of between about 2-500 blocks, wherein the blocks may be the same or different. In another embodiment a stabilizing polymer comprised of a block copolymer may be comprised of between about 2-250 blocks, wherein the blocks may be the same or different. In another embodiment a stabilizing polymer comprised of a block copolymer may be comprised of between about 2-100 blocks, wherein the blocks may be the same or different. In another embodiment a stabilizing polymer comprised of a block copolymer may be comprised of between about 2-50 blocks, wherein the blocks may be the same or different. In another embodiment a stabilizing polymer comprised of a block copolymer may be comprised of between about 2-20 blocks, wherein the blocks may be the same or different. In another embodiment a stabilizing polymer comprised of a block copolymer may be comprised of between about 2-10 blocks, wherein the blocks may be the same or different. In another embodiment a stabilizing polymer comprised of a block copolymer may be comprised of between about 2-5 blocks, wherein the blocks may be the same or different.

In another embodiment a stabilizing polymer comprised of a block copolymer may be comprised of between about 5-10,000 blocks, wherein the blocks may be the same or different. In another embodiment a stabilizing polymer comprised of a block copolymer may be comprised of between about 5-1,000 blocks, wherein the blocks may be the same or different. In another embodiment a stabilizing polymer comprised of a block copolymer may be comprised of between about 5-500 blocks, wherein the blocks may be the same or different. In another embodiment a stabilizing polymer comprised of a block copolymer may be comprised of between about 5-250 blocks, wherein the blocks may be the same or different. In another embodiment a stabilizing polymer comprised of a block copolymer may be comprised of between about 5-100 blocks, wherein the blocks may be the same or different. In another embodiment a stabilizing polymer comprised of a block copolymer may be comprised of between about 5-50 blocks, wherein the blocks may be the same or different. In another embodiment a stabilizing polymer comprised of a block copolymer may be comprised of between about 5-20 blocks, wherein the blocks may be the same or different. In another embodiment a stabilizing polymer comprised of a block copolymer may be comprised of between about 5-10 blocks, wherein the blocks may be the same or different.

In another embodiment a stabilizing polymer comprised of a block copolymer may be comprised of between about 10-10,000 blocks, wherein the blocks may be the same or different. In another embodiment a stabilizing polymer comprised of a block copolymer may be comprised of between about 10-1,000 blocks, wherein the blocks may be the same or different. In another embodiment a stabilizing polymer comprised of a block copolymer may be comprised of between about 10-500 blocks, wherein the blocks may be the same or different. In another embodiment a stabilizing polymer comprised of a block copolymer may be comprised of between about 10-250 blocks, wherein the blocks may be the same or different. In another embodiment a stabilizing polymer comprised of a block copolymer may be comprised of between about 10-100 blocks, wherein the blocks may be the same or different. In another embodiment a stabilizing polymer comprised of a block copolymer may be comprised of between about 10-50 blocks, wherein the blocks may be the same or different. In another embodiment a stabilizing polymer comprised of a block copolymer may be comprised of between about 10-20 blocks, wherein the blocks may be the same or different.

In one embodiment a stabilizing polymer may be branched. In one embodiment a stabilizing polymer may comprise one or more branches. In one embodiment one or more backbone chain of a stabilizing polymer may comprise one or more branches. In one embodiment one or more backbone repeat units of a stabilizing polymer may comprise one or more branches.

In one embodiment a stabilizing polymer may comprise one or more sidechains, pendant groups, or side groups

In one embodiment a backbone chain of a stabilizing polymer may comprise one or more sidechains, pendant groups, or side groups.

In one embodiment one or more backbone repeat units of a stabilizing polymer may comprise one or more sidechains, pendant groups, or side groups.

In one embodiment a stabilizing polymer may be comprised of one or more carbon atoms. In another embodiment a stabilizing polymer may be comprised of one or more nitrogen atoms. In another embodiment a stabilizing polymer may be comprised of one or more oxygen atoms. In another embodiment a stabilizing polymer may be comprised of one or more sulfur atoms. In another embodiment a stabilizing polymer may be comprised of one or more phosphorus atoms.

In one embodiment a backbone chain of a stabilizing polymer may be comprised of one or more carbon atoms. In another embodiment a backbone chain of a stabilizing polymer may be comprised of one or more nitrogen atoms. In another embodiment a backbone chain of a stabilizing polymer may be comprised of one or more oxygen atoms. In another embodiment a backbone chain of a stabilizing polymer may be comprised of one or more sulfur atoms. In another embodiment a backbone chain of a stabilizing polymer may be comprised of one or more phosphorus atoms.

In one embodiment one or more backbone repeat unit of a stabilizing polymer may be comprised of one or more carbon atoms. In another embodiment one or more backbone repeat unit of a stabilizing polymer may be comprised of one or more nitrogen atoms. In another embodiment one or more backbone repeat unit of a stabilizing polymer may be comprised of one or more oxygen atoms. In another embodiment one or more backbone repeat unit of a stabilizing polymer may be comprised of one or more sulfur atoms. In another embodiment one or more backbone repeat unit of a stabilizing polymer may be comprised of one or more phosphorus atoms.

### Repeat Unit Composition

In another embodiment a stabilizing polymer may be comprised of one or more backbone chain wherein one or more backbone repeat unit may be comprised of at least one carbon atom. In one embodiment a backbone repeat unit may comprise C1-100 alkyl, alkenyl, alkynyl, aryl, or aralkyl group, each of which may contain up to 90 heteroatoms. In another embodiment a backbone repeat unit may comprise C1-80 alkyl, alkenyl, alkynyl, aryl, or aralkyl group, each of which may contain up to 75 heteroatoms. In another embodiment a backbone repeat unit may comprise C1-60 alkyl, alkenyl, alkynyl, aryl, or aralkyl group, each of which may contain up to 55 heteroatoms. In another embodiment a backbone repeat unit may comprise C1-50 alkyl, alkenyl, alkynyl, aryl, or aralkyl group, each of which may contain up to 45 heteroatoms. In another embodiment a backbone repeat unit may comprise C1-40 alkyl, alkenyl, alkynyl, aryl, or aralkyl group, each of which may contain up to 36 heteroatoms. In another embodiment a backbone repeat unit may comprise C1-30 alkyl, alkenyl, alkynyl, aryl, or aralkyl group, each of which may contain up to 27 heteroatoms. In another embodiment a backbone repeat unit may comprise C1-20 alkyl, alkenyl, alkynyl, aryl, or aralkyl group, each of which may contain up to 18 heteroatoms. In another embodiment a backbone repeat unit may comprise C1-20 alkyl, alkenyl, alkynyl, aryl, or aralkyl group, each of which may contain up to 10 heteroatoms. In another embodiment a backbone repeat unit may comprise C1-18 alkyl, alkenyl, alkynyl, aryl, or aralkyl group, each of which may contain up to 16 heteroatoms. In another embodiment a backbone repeat unit may comprise C1-18 alkyl, alkenyl, alkynyl, aryl, or aralkyl group, each of which may contain up to 9 heteroatoms. In another embodiment a backbone repeat unit may comprise C1-16 alkyl, alkenyl, alkynyl, aryl, or aralkyl group, each of which may contain up to 14 heteroatoms. In another embodiment a backbone repeat unit may comprise C1-18 alkyl, alkenyl, alkynyl, aryl, or aralkyl group, each of which may contain up to 16 heteroatoms. In another embodiment a backbone repeat unit may comprise C1-16 alkyl, alkenyl, alkynyl, aryl, or aralkyl group, each of which may contain up to 8 heteroatoms. In another embodiment a backbone repeat unit may comprise C1-14 alkyl, alkenyl, alkynyl, aryl, or aralkyl group, each of which may contain up to 12 heteroatoms. In another embodiment a backbone repeat unit may comprise C1-14 alkyl, alkenyl, alkynyl, aryl, or aralkyl group, each of which may contain up to 7 heteroatoms. In another embodiment a backbone repeat unit may comprise C1-12 alkyl, alkenyl, alkynyl, aryl, or aralkyl group, each of which may contain up to 10 heteroatoms. In another embodiment a backbone repeat unit may comprise C1-12 alkyl, alkenyl, alkynyl, aryl, or aralkyl group, each of which may contain up to 6 heteroatoms. In another embodiment a backbone repeat unit may comprise C1-10 alkyl, alkenyl, alkynyl, aryl, or aralkyl group, each of which may contain up to 9 heteroatoms. In another embodiment a backbone repeat unit may comprise C1-10 alkyl, alkenyl, alkynyl, aryl, or aralkyl group, each of which may contain up to 5 heteroatoms. In another embodiment a backbone repeat unit may comprise C1-8 alkyl, alkenyl, alkynyl, aryl, or aralkyl group, each of which may contain up to 7 heteroatoms. In another embodiment a backbone repeat unit may comprise C1-8 alkyl, alkenyl, alkynyl, aryl, or aralkyl group, each of which may contain up to 4 heteroatoms. In another embodiment a backbone repeat unit may comprise C1-6 alkyl, alkenyl, alkynyl, aryl, or aralkyl group, each of which may contain up to 5 heteroatoms. In another embodiment a backbone repeat unit may comprise C1-6 alkyl, alkenyl, alkynyl, aryl, or aralkyl group, each of which may contain up to 3 heteroatoms.

In another embodiment, a backbone repeat unit may comprise C1-100 with up to 90 heteroatoms. In another embodiment a backbone repeat unit may comprise C1-90 with up to 85 heteroatoms. In another embodiment a backbone repeat unit may comprise C1-80 with up to 75 heteroatoms. In another embodiment a backbone repeat unit may comprise C1-70 with up to 65 heteroatoms. In another embodiment a backbone repeat unit may comprise C1-60 with up to 55 heteroatoms. In another embodiment a backbone repeat unit may comprise C1-50 with up to 45 heteroatoms. In another embodiment a backbone repeat unit may comprise C1-40 with up to 36 heteroatoms. In another embodiment a backbone repeat unit may comprise C1-30 with up to 27 heteroatoms. In another embodiment a backbone repeat unit may comprise C1-20 with up to 18 heteroatoms. In another embodiment a backbone repeat unit may comprise C1-20 with up to 10 heteroatoms. In another embodiment a backbone repeat unit may comprise C1-18 with up to 16 heteroatoms. In another embodiment a backbone repeat unit may comprise C1-18 with up to 9 heteroatoms. In another embodiment a backbone repeat unit may comprise C1-16 with up to 14 heteroatoms. In another embodiment a backbone repeat unit may comprise C1-16 with up to 8 heteroatoms. In another embodiment a backbone repeat unit may comprise C1-14 with up to 12 heteroatoms. In another embodiment a backbone repeat unit may comprise C1-14 with up to 7 heteroatoms. In another embodiment a backbone repeat unit may comprise C1-12 with up to 10 heteroatoms. In another embodiment a backbone repeat unit may comprise C1-12 with up to 6 heteroatoms. In another embodiment a backbone repeat unit may comprise C1-10 with up to 9 heteroatoms. In another embodiment a backbone repeat unit may comprise C1-10 with up to 5 heteroatoms. In another embodiment a backbone repeat unit may comprise C1-8 with up to 7 heteroatoms. In another embodiment a backbone repeat unit may comprise C1-8 with up to 4 heteroatoms. In another embodiment a backbone repeat unit may comprise C1-6 with up to 5 heteroatoms. In another embodiment a backbone repeat unit may comprise C1-6 with up to 3 heteroatoms.

In other embodiments one or more backbone repeat unit of a stabilizing polymer may comprise one or more groups comprising at least one carbon, hydrogen, nitrogen, oxygen, sulfur, or phosphorus including, but not limited to, as non-limiting examples: hydroxy groups, alcohols, alkyl groups, alkenyl groups, alkynyl groups, aryl groups, aralkyl groups, ether groups, ethers, carboxamide groups, amides, amines, imines, disulfide groups, sulfonyl groups, sulfones, carboalkoxy groups, esters, carbonate esters, carboxylate esters, carboxylic acid groups, carboxylate groups, organophosphate groups, phosphate ester groups, phosphodiester groups, phosphonic acid groups, phosphonate groups, sulfonic acid groups, sulfonate groups, or organosulfate groups.

### Branches

In one embodiment a stabilizing polymer may be linear. In one embodiment a stabilizing polymer may not be branched. In one embodiment a backbone chain of a stabilizing polymer may be linear. In one embodiment a backbone chain of a stabilizing polymer may not be branched.

In one embodiment a stabilizing polymer may comprise one or more branches. In one embodiment a stabilizing polymer may comprise one or more dendrimer.

In another embodiment, a stabilizing polymer may comprise up to about 1,000 branches. In another embodiment, a stabilizing polymer may comprise up to about 500 branches. In another embodiment, a stabilizing polymer may comprise up to about 250 branches. In another embodiment, a stabilizing polymer may comprise up to about 100 branches. In another embodiment, a stabilizing polymer may comprise up to about 50 branches. In another embodiment, a stabilizing polymer may comprise up to about 45 branches. In another embodiment, a stabilizing polymer may comprise up to about 40 branches. In another embodiment, a stabilizing polymer may comprise up to about 35 branches. In another embodiment, a stabilizing polymer may comprise up to about 30 branches. In another embodiment, a stabilizing polymer may comprise up to about 25 branches. In another embodiment, a stabilizing polymer may comprise up to about 20 branches. In another embodiment, a stabilizing polymer may comprise up to about 15 branches. In another embodiment, a stabilizing polymer may comprise up to about 10 branches. In another embodiment, a stabilizing polymer may comprise up to about 5 branches.

In one embodiment one or more backbone chain of a stabilizing polymer may comprise one or more branches.

In another embodiment, a backbone chain of a stabilizing polymer may comprise up to about 1,000 branches. In another embodiment, a backbone chain of a stabilizing polymer may comprise up to about 500 branches. In another embodiment, a backbone chain of a stabilizing polymer may comprise up to about 250 branches. In another embodiment, a backbone chain of a stabilizing polymer may comprise up to about 100 branches. In another embodiment, a backbone chain of a stabilizing polymer may comprise up to about 50 branches. In another embodiment, a backbone chain of a stabilizing polymer may comprise up to about 45 branches. In another embodiment, a backbone chain of a stabilizing polymer may comprise up to about 40 branches. In another embodiment, a backbone chain of a stabilizing polymer may comprise up to about 35 branches. In another embodiment, a backbone chain of a stabilizing polymer may comprise up to about 30 branches. In another embodiment, a backbone chain of a stabilizing polymer may comprise up to about 25 branches. In another embodiment, a backbone chain of a stabilizing polymer may comprise up to about 20 branches. In another embodiment, a backbone chain of a stabilizing polymer may comprise up to about 15 branches. In another embodiment, a backbone chain of a stabilizing polymer may comprise up to about 10 branches. In another embodiment, a backbone chain of a stabilizing polymer may comprise up to about 5 branches.

In one embodiment, a branch may comprise at least one carbon atom, wherein a branch may comprise C1-5000 with up to 4500 heteroatoms. In another embodiment a branch may comprise C1-2500 with up to 2300 heteroatoms. In another embodiment a branch may comprise C1-1000 with up to 900 heteroatoms. In another embodiment a branch may comprise C1-1000 with up to 500 heteroatoms. In another embodiment a branch may comprise C1-900 with up to 850 heteroatoms. In another embodiment a branch may comprise C1-900 with up to 450 heteroatoms. In another embodiment a branch may comprise C1-800 with up to 750 heteroatoms. In another embodiment a branch may comprise C1-800 with up to 400 heteroatoms. In another embodiment a branch may comprise C1-700 with up to 650 heteroatoms. In another embodiment a branch may comprise C1-700 with up to 350 heteroatoms. In another embodiment a branch may comprise C1-600 with up to 550 heteroatoms. In another embodiment a branch may comprise C1-600 with up to 300 heteroatoms. In another embodiment a branch may comprise C1-500 with up to 450 heteroatoms. In another embodiment a branch may comprise C1-500 with up to 250 heteroatoms. In another embodiment a branch may comprise C1-400 with up to 360 heteroatoms. In another embodiment a branch may comprise C1-400 with up to 200 heteroatoms. In another embodiment a branch may comprise C1-300 with up to 270 heteroatoms. In another embodiment a branch may comprise C1-300 with up to 150 heteroatoms. In another embodiment a branch may comprise C1-200 with up to 180 heteroatoms. In another embodiment a branch may comprise C1-200 with up to 100 heteroatoms. In another embodiment a branch may comprise C1-150 with up to 140 heteroatoms. In another embodiment a branch may comprise C1-150 with up to 75 heteroatoms. In another embodiment a branch may comprise C1-100 with up to 90 heteroatoms. In another embodiment a branch may comprise C1-100 with up to 50 heteroatoms. In another embodiment a branch may comprise C1-50 with up to 45 heteroatoms. In another embodiment a branch may comprise C1-50 with up to 25 heteroatoms. In another embodiment a branch may comprise C1-30 with up to 27 heteroatoms. In another embodiment a branch may comprise C1-30 with up to 15 heteroatoms. In another embodiment a branch may comprise C1-20 with up to 18 heteroatoms. In another embodiment a branch may comprise C1-20 with up to 10 heteroatoms. In another embodiment a branch may comprise C1-10 with up to 9 heteroatoms. In another embodiment a branch may comprise C1-10 with up to 5 heteroatoms

In other embodiments one or more branch of a stabilizing polymer may comprise one or more groups comprising at least one carbon, hydrogen, nitrogen, oxygen, sulfur, or phosphorus including, but not limited to, as non-limiting examples: hydroxy groups, alcohols, alkyl groups, alkenyl groups, alkynyl groups, aryl groups, aralkyl groups, ether groups, ethers, carboxamide groups, amides, amines, imines, disulfide groups, sulfonyl groups, sulfones, carboalkoxy groups, esters, carbonate esters, carboxylate esters, carboxylic acid groups, carboxylate groups, organophosphate groups, phosphate ester groups, phosphodiester groups, phosphonic acid groups, phosphonate groups, sulfonic acid groups, sulfonate groups, or organosulfate groups.

In another embodiment, a stabilizing polymer may have a molecular weight between about 100-100,000,000 daltons. In another embodiment, a stabilizing polymer may have a molecular weight between about 100-10,000,000 daltons. In another embodiment, a stabilizing polymer may have a molecular weight between about 100-1,000,000 daltons. In another embodiment, a stabilizing polymer may have a molecular weight between about 100-800,000 daltons. In another embodiment, a stabilizing polymer may have a molecular weight between about 100-600,000 daltons. In another embodiment, a stabilizing polymer may have a molecular weight between about 100-400,000 daltons. In another embodiment, a stabilizing polymer may have a molecular weight between about 100-200,000 daltons. In another embodiment, a stabilizing polymer may have a molecular weight between about 100-100,000 daltons. In another embodiment, a stabilizing polymer may have a molecular weight between about 100-80,000 daltons. In another embodiment, a stabilizing polymer may have a molecular weight between about 100-60,000 daltons. In another embodiment, a stabilizing polymer may have a molecular weight between about 100-50,000 daltons. In another embodiment, a stabilizing polymer may have a molecular weight between about 100-45,000 daltons. In another embodiment, a stabilizing polymer may have a molecular weight between about 100-40,000 daltons. In another embodiment, a stabilizing polymer may have a molecular weight between about 100-35,000 daltons. In another embodiment, a stabilizing polymer may have a molecular weight between about 100-30,000 daltons. In another embodiment, a stabilizing polymer may have a molecular weight between about 100-25,000 daltons. In another embodiment, a stabilizing polymer may have a molecular weight between about 100-20,000 daltons. In another embodiment, a stabilizing polymer may have a molecular weight between about 100-15,000 daltons. In another embodiment, a stabilizing polymer may have a molecular weight between about 100-10,000 daltons. In another embodiment, a stabilizing polymer may have a molecular weight between about 100-5,000 daltons. In another embodiment, a stabilizing polymer may have a molecular weight between about 100-2,000 daltons. In another embodiment, a stabilizing polymer may have a molecular weight between about 100-1,000 daltons.

In another embodiment, a stabilizing polymer may have a molecular weight between about 1,000-100,000,000 daltons. In another embodiment, a stabilizing polymer may have a molecular weight between about 1,000-10,000,000 daltons. In another embodiment, a stabilizing polymer may have a molecular weight between about 1,000-1,000,000 daltons. In another embodiment, a stabilizing polymer may have a molecular weight between about 1,000-800,000 daltons. In another embodiment, a stabilizing polymer may have a molecular weight between about 1,000-600,000 daltons. In another embodiment, a stabilizing polymer may have a molecular weight between about 1,000-400,000 daltons. In another embodiment, a stabilizing polymer may have a molecular weight between about 1,000-200,000 daltons. In another embodiment, a stabilizing polymer may have a molecular weight between about 1,000-100,000 daltons. In another embodiment, a stabilizing polymer may have a molecular weight between about 1,000-80,000 daltons. In another embodiment, a stabilizing polymer may have a molecular weight between about 1,000-60,000 daltons. In another embodiment, a stabilizing polymer may have a molecular weight between about 1,000-50,000 daltons. In another embodiment, a stabilizing polymer may have a molecular weight between about 1,000-45,000 daltons. In another embodiment, a stabilizing polymer may have a molecular weight between about 1,000-40,000 daltons. In another embodiment, a stabilizing polymer may have a molecular weight between about 1,000-35,000 daltons. In another embodiment, a stabilizing polymer may have a molecular weight between about 1,000-30,000 daltons. In another embodiment, a stabilizing polymer may have a molecular weight between about 1,000-25,000 daltons. In another embodiment, a stabilizing polymer may have a molecular weight between about 1,000-20,000 daltons. In another embodiment, a stabilizing polymer may have a molecular weight between about 1,000-15,000 daltons. In another embodiment, a stabilizing polymer may have a molecular weight between about 1,000-10,000 daltons. In another embodiment, a stabilizing polymer may have a molecular weight between about 1,000-5,000 daltons. In another embodiment, a stabilizing polymer may have a molecular weight between about 1,000-2,000 daltons.

In another embodiment, a stabilizing polymer may have a molecular weight between about 5,000-100,000,000 daltons. In another embodiment, a stabilizing polymer may have a molecular weight between about 5,000-10,000,000 daltons. In another embodiment, a stabilizing polymer may have a molecular weight between about 5,000-1,000,000 daltons. In another embodiment, a stabilizing polymer may have a molecular weight between about 5,000-800,000 daltons. In another embodiment, a stabilizing polymer may have a molecular weight between about 5,000-600,000 daltons. In another embodiment, a stabilizing polymer may have a molecular weight between about 5,000-400,000 daltons. In another embodiment, a stabilizing polymer may have a molecular weight between about 5,000-200,000 daltons. In another embodiment, a stabilizing polymer may have a molecular weight between about 5,000-100,000 daltons. In another embodiment, a stabilizing polymer may have a molecular weight between about 5,000-80,000 daltons. In another embodiment, a stabilizing polymer may have a molecular weight between about 5,000-60,000 daltons. In another embodiment, a stabilizing polymer may have a molecular weight between about 5,000-50,000 daltons. In another embodiment, a stabilizing polymer may have a molecular weight between about 5,000-45,000 daltons. In another embodiment, a stabilizing polymer may have a molecular weight between about 5,000-40,000 daltons. In another embodiment, a stabilizing polymer may have a molecular weight between about 5,000-35,000 daltons. In another embodiment, a stabilizing polymer may have a molecular weight between about 5,000-30,000 daltons. In another embodiment, a stabilizing polymer may have a molecular weight between about 5,000-25,000 daltons. In another embodiment, a stabilizing polymer may have a molecular weight between about 5,000-25,000 daltons. In another embodiment, a stabilizing polymer may have a molecular weight between about 5,000-20,000 daltons. In another embodiment, a stabilizing polymer may have a molecular weight between about 5,000-15,000 daltons. In another embodiment, a stabilizing polymer may have a molecular weight between about 5,000-10,000 daltons.

In another embodiment, a stabilizing polymer may have a molecular weight between about 10,000-100,000,000 daltons. In another embodiment, a stabilizing polymer may have a molecular weight between about 10,000-10,000,000 daltons. In another embodiment, a stabilizing polymer may have a molecular weight between about 10,000-1,000,000 daltons. In another embodiment, a stabilizing polymer may have a molecular weight between about 10,000-800,000 daltons. In another embodiment, a stabilizing polymer may have a molecular weight between about 10,000-600,000 daltons. In another embodiment, a stabilizing polymer may have a molecular weight between about 10,000-400,000 daltons. In another embodiment, a stabilizing polymer may have a molecular weight between about 10,000-200,000 daltons. In another embodiment, a stabilizing polymer may have a molecular weight between about 10,000-100,000 daltons. In another embodiment, a stabilizing polymer may have a molecular weight between about 10,000-80,000 daltons. In another embodiment, a stabilizing polymer may have a molecular weight between about 10,000-60,000 daltons. In another embodiment, a stabilizing polymer may have a molecular weight between about 10,000-50,000 daltons. In another embodiment, a stabilizing polymer may have a molecular weight between about 10,000-45,000 daltons. In another embodiment, a stabilizing polymer may have a molecular weight between about 10,000-40,000 daltons. In another embodiment, a stabilizing polymer may have a molecular weight between about 10,000-35,000 daltons. In another embodiment, a stabilizing polymer may have a molecular weight between about 10,000-30,000 daltons. In another embodiment, a stabilizing polymer may have a molecular weight between about 10,000-25,000 daltons.

In another embodiment, a stabilizing polymer may have a molecular weight between about 20,000-100,000,000 daltons. In another embodiment, a stabilizing polymer may have a molecular weight between about 20,000-10,000,000 daltons. In another embodiment, a stabilizing polymer may have a molecular weight between about 20,000-1,000,000 daltons. In another embodiment, a stabilizing polymer may have a molecular weight between about 20,000-800,000 daltons. In another embodiment, a stabilizing polymer may have a molecular weight between about 20,000-600,000 daltons. In another embodiment, a stabilizing polymer may have a molecular weight between about 20,000-400,000 daltons. In another embodiment, a stabilizing polymer may have a molecular weight between about 20,000-200,000 daltons. In another embodiment, a stabilizing polymer may have a molecular weight between about 20,000-100,000 daltons. In another embodiment, a stabilizing polymer may have a molecular weight between about 20,000-80,000 daltons. In another embodiment, a stabilizing polymer may have a molecular weight between about 20,000-60,000 daltons. In another embodiment, a stabilizing polymer may have a molecular weight between about 20,000-50,000 daltons. In another embodiment, a stabilizing polymer may have a molecular weight between about 20,000-45,000 daltons. In another embodiment, a stabilizing polymer may have a molecular weight between about 20,000-40,000 daltons.

In another embodiment, a stabilizing polymer may have a molecular weight between about 50,000-100,000,000 daltons. In another embodiment, a stabilizing polymer may have a molecular weight between about 50,000-10,000,000 daltons. In another embodiment, a stabilizing polymer may have a molecular weight between about 50,000-1,000,000 daltons. In another embodiment, a stabilizing polymer may have a molecular weight between about 50,000-800,000 daltons. In another embodiment, a stabilizing polymer may have a molecular weight between about 50,000-600,000 daltons. In another embodiment, a stabilizing polymer may have a molecular weight between about 50,000-400,000 daltons. In another embodiment, a stabilizing polymer may have a molecular weight between about 50,000-200,000 daltons. In another embodiment, a stabilizing polymer may have a molecular weight between about 50,000-100,000 daltons. In another embodiment, a stabilizing polymer may have a molecular weight between about 50,000-80,000 daltons.

In another embodiment, a stabilizing polymer may have a molecular weight between about 100,000-100,000,000 daltons. In another embodiment, a stabilizing polymer may have a molecular weight between about 100,000-10,000,000 daltons. In another embodiment, a stabilizing polymer may have a molecular weight between about 100,000-1,000,000 daltons. In another embodiment, a stabilizing polymer may have a molecular weight between about 100,000-800,000 daltons. In another embodiment, a stabilizing polymer may have a molecular weight between about 100,000-600,000 daltons. In another embodiment, a stabilizing polymer may have a molecular weight between about 100,000-400,000 daltons. In another embodiment, a stabilizing polymer may have a molecular weight between about 100,000-200,000 daltons.

In another embodiment, a stabilizing polymer may have a molecular weight greater than about 100 daltons. In another embodiment, a stabilizing polymer may have a molecular weight greater than about 500 daltons. In another embodiment, a stabilizing polymer may have a molecular weight greater than about 1,000 daltons. In another embodiment, a stabilizing polymer may have a molecular weight greater than about 2,000 daltons. In another embodiment, a stabilizing polymer may have a molecular weight greater than about 5,000 daltons. In another embodiment, a stabilizing polymer may have a molecular weight greater than about 10,000 daltons. In another embodiment, a stabilizing polymer may have a molecular weight greater than about 20,000 daltons. In another embodiment, a stabilizing polymer may have a molecular weight greater than about 30,000 daltons. In another embodiment, a stabilizing polymer may have a molecular weight greater than about 40,000 daltons. In another embodiment, a stabilizing polymer may have a molecular weight greater than about 50,000 daltons. In another embodiment, a stabilizing polymer may have a molecular weight greater than about 100,000 daltons. In another embodiment, a stabilizing polymer may have a molecular weight greater than about 200,000 daltons. In another embodiment, a stabilizing polymer may have a molecular weight greater than about 500,000 daltons.

In one embodiment a stabilizing polymer may be comprised of one or more backbone repeat units.

In one embodiment one or more backbone repeat units of a stabilizing polymer may be comprised of one or more acrylate or polyacrylate, including, but not limited to, one or more of the following non-limiting examples: acrylate, polyacrylate, poly(methyl acrylate), poly(methacrylate), poly(methyl methacrylate), poly(acrylic acid), poly(methacrylic acid), poly(hydroxyethyl methacrylate), poly(ethyl methacrylate), poly(butyl methacrylate), poly(methoxy ethylacrylate), methyl acrylate, methacrylate, methyl methacrylate, acrylic acid, methacrylic acid, hydroxyethyl methacrylate, ethyl methacrylate, butyl methacrylate, or methoxy ethylacrylate, or combinations thereof.

In one embodiment one or more backbone repeat units of a stabilizing polymer may be comprised of one or more ester or polyester (such as a carboxylate ester or carbonate ester as non-limiting examples) including, but not limited to, one or more of the following non-limiting examples: carboxylate ester, carbonate ester, polyester, polycarbonate, poly(lactic acid), poly(lactide), poly(glycolide), poly(glycolic acid), poly(lactide-co-glycolide), poly(lactic-co-glycolic acid) polycaprolactone, poly-ε-caprolactone, poly(ethylene glycol)-block-poly(caprolactone), polycaprolactone diol, poly(ethylene glycol)-block-poly(lactic acid), poly(hydroxyalkanoate), poly(hydroxybutyrate), poly[(R)-3-hydroxybutyric acid), poly(3-hydroxybutyrate), poly(3-hydroxybutyrate-co-3-hydroxyvalerate), poly(hydroxy valerate), poly(trimethylene carbonate), poly(ethylene carbonate), poly(propylene carbonate), poly(dioxanone), poly(p-dioxanone), poly(adipic acid), poly(adipate), poly(succinic acid), poly(succinate), poly(fumarate), poly(caprolactone-block-fumarate), polycaprolactone fumarate, poly(ethylene adipate), poly(butylene succinate), poly(ethylene glutarate), poly(glutarate), poly(ethylene glycol diacetate), poly(ethylene azelate), poly(trimethylene glutarate), poly(dioxanone), lactic acid, lactide, glycolide, glycolic acid, caprolactone, ε-caprolactone, hydroxyalkanoate, hydroxybutyrate, 3-hydroxybutyric acid, butyrate, trimethylene carbonate, ethylene carbonate, propylene carbonate, dioxanone, p-dioxanone, adipic acid, adipate, succinic acid, succinate, fumarate, ethylene adipate, butylene succinate, ethylene glutarate, glutarate, ethylene glycol diacetate, ethylene azelate, trimethylene glutarate, or dioxanone, or combinations thereof. As a non-limiting example an ester may be formed by linking one or more backbone repeat unit to an adjacent backbone repeat unit.

In one embodiment one or more backbone repeat units of a stabilizing polymer may be comprised of one or more ether or polyether, including, but not limited to, one or more of the following non-limiting examples: polyether, poly(ethylene oxide-block-propylene oxide), poly(ether ether ketone), PEEK, poly(ethylene glycol), poly(propylene glycol), poly(propylene oxide glycol), poly(ethylene oxide), poly(ethylene oxide glycol), poly(propylene oxide), poly(tetramethylene glycol), poly(butylene oxide glycol), poly(glycerol), oxetan, poly(tetrahydrofuran), poly(dioxane), poly(dioxanone), ethylene glycol, propylene glycol, propylene oxide glycol, ethylene oxide, ethylene oxide glycol, propylene oxide, tetramethylene glycol, butylene oxide glycol, glycerol, oxetan, tetrahydrofuran, dioxane, or dioxanone, or combinations thereof. As a non-limiting example, an ether may be formed by linking one or more backbone repeat unit to an adjacent backbone repeat unit.

In one embodiment one or more backbone repeat units of a stabilizing polymer may be comprised of one or more amide or polyamide, including, but not limited to, one or more of the following non-limiting examples: amide, polyamide, nylon, poly(hexamethylene adipamide), nylon 6/6, nylon 6/10, nylon 10/10, nylon 10/12, nylon 11, nylon 12, acrylamide, poly(amino acid), peptide, poly(peptide), poly(aspartic acid), poly(glutamic acid), poly(lysine), poly(histidine), poly(tyrosine), poly(methionine), poly(glycine), poly(proline), poly(cysteine), amino acid, peptide, aspartic acid, glutamic acid, lysine, histidine, tyrosine, methionine, glycine, proline, or cysteine, or combinations thereof.

In one embodiment one or more backbone repeat units of a stabilizing polymer may be comprised of one or more vinyl or polyvinyl, including but not limited to, one or more of the following non-limiting examples: vinyl, polyvinyl, poly(2-vinylpyridine N-oxide), poly(2-vinyl-1-methylpyridinium), poly(1-methyl-4-vinylpyridinium), poly(4-vinyl-1-methylpyridinium), poly(4-vinyl N-methyl pyridinium), poly(vinylsulfonic acid), poly(vinylsulfonate), poly(N-vinylpyrrolidone-block-vinyl acetate), poly(vinylphosphonic acid), poly(vinylphosphonate), poly(4-vinylphenol), poly(2-vinylpyridine), poly(4-vinylpyridine), polyethylene, polypropylene, polystyrene, poly(styrene sulfonate), poly(vinyl chloride), poly(vinyl acetate), polyacrylonitrile, poly(N-vinylpyrrolidone), poly(vinylpyrrolidone), poly(vinyl sulfate), poly(vinyl sulfonate), poly(vinyl alcohol), poly(diallyldimethylammonium), diallyldimethylammonium, ethylene, propylene, styrene, styrene sulfonate, vinyl chloride, vinyl acetate, acrylonitrile, N-vinylpyrrolidone, vinyl sulfate, vinyl sulfonate, 2-vinylpyridine N-oxide, 2-vinyl-1-methylpyridinium, 1-methyl-4-vinylpyridinium, 4-vinyl-1-methylpyridinium, 4-vinyl N-methyl pyridinium, vinylsulfonic acid, vinylsulfonate, vinyl acetate, vinylphosphonic acid, vinylphosphonate, 4-vinylphenol, 2-vinylpyridine, 4-vinylpyridine, or vinyl alcohol, or combinations thereof.

In one embodiment one or more backbone repeat units of a stabilizing polymer may be comprised of one or more oxazoline or poly(oxazoline), such as 2-ethyl-2-oxazoline or poly(2-ethyl-2-oxazoline) as a non-limiting example.

In one embodiment one or more backbone repeat units of a stabilizing polymer may be comprised of one or more acrylamide or polyacrylamide.

In one embodiment one or more backbone repeat units of a stabilizing polymer may be comprised of one or more diallyldimethylammonium or polydiallyldimethylammonium.

In one embodiment one or more backbone repeat units of a stabilizing polymer may be comprised of one or more cyclic amide, such as N-vinylpyrrolidone or poly(vinylpyrrolidone).

In one embodiment one or more backbone repeat units of a stabilizing polymer may be comprised of one or more phosphoester or polyphosphoester, such as an organophosphate or phosphodiester as non-limiting examples.

In one embodiment one or more backbone repeat units of a stabilizing polymer may be comprised of one or more phosphazene or poly(phosphazene), such as an organophosphazene or poly(organophosphazene).

In one embodiment one or more backbone repeat units of a stabilizing polymer may be comprised of one or more siloxane or polysiloxane, such as dimethyl siloxane or polydimethylsiloxane as non-limiting examples.

In one embodiment one or more backbone repeat units of a stabilizing polymer may be comprised of one or more imide or polyimide.

In one embodiment one or more backbone repeat units of a stabilizing polymer may be comprised of one or more urea or polyurea.

In one embodiment one or more backbone repeat units of a stabilizing polymer may be comprised of one or more amine or polyamine.

In one embodiment one or more backbone repeat units of a stabilizing polymer may be comprised of one or more amine or polyamine, wherein an amine or polyamine may comprise a quaternary ammonium cation, such as hexadimethrine as a non-limiting example.

In one embodiment one or more backbone repeat units of a stabilizing polymer may be comprised of one or more amine or polyamine, wherein an amine or polyamine may comprise a tertiary amine.

In one embodiment one or more backbone repeat units of a stabilizing polymer may be comprised of one or more isocyanate, such as polyurethane as a non-limiting example.

In one embodiment one or more backbone repeat units of a stabilizing polymer may comprise one or more combinations of one or more of the following: acrylate, polyacrylate, ester, polyester, ether, polyether, amide, polyamide, vinyl, polyvinyl, oxazoline, poly(oxazoline) acrylamide, polyacrylamide, diallyldimethylammonium, polydiallyldimethylammonium, cyclic amide, poly(vinylpyrrolidone), phosphoester, polyphosphoester, phosphazene, poly(phosphazene), organophosphazene, poly(organophosphazene), siloxane, polysiloxane, imide, polyimide, urea, polyurea, amine, polyamine, quaternary ammonium amine, quaternary ammonium polyamine, tertiary amine, tertiary amine polyamine, isocyanate, or polyurethane or combinations thereof.

In one embodiment one or more blocks of a stabilizing polymer comprised of a block copolymer may be comprised of one or more backbone repeat unit, wherein a backbone repeat unit may be at least part of one or more block of a block copolymer or multiblock polymer. In one embodiment one or more backbone repeat unit of a stabilizing polymer comprised of one or more copolymer (such as a random copolymer or alternating copolymer as non-limiting examples) may be comprised of one or more backbone repeat unit, wherein a backbone repeat unit may be at least part of one or more copolymer.

In one embodiment a stabilizing polymer may be comprised of one or more type of backbone repeat unit.

In one embodiment a stabilizing polymer may be comprised of two or more types of backbone repeat units, wherein the stabilizing polymer may be comprised of a copolymer such as a bipolymer, terpolymer, or quaterpolymer, or higher order copolymer, such as a linear copolymer, a random copolymer, an alternating copolymer, a block copolymer, a statistical copolymer, a gradient copolymer, a periodic copolymer, or a sequential copolymer, as non-limiting examples.

In one embodiment a stabilizing polymer may be comprised of two or more types of backbone repeat units, wherein the stabilizing polymer may be comprised of a block copolymer, such as a diblock polymer, triblock polymer, quaterblock polymer, or higher order multiblock polymer, such as a linear block copolymer, a random block copolymer, or an alternating block copolymer, as non-limiting examples.

In one embodiment a backbone repeat unit of a stabilizing polymer may not be comprised of an amino acid. In one embodiment a backbone repeat unit of a stabilizing polymer may not be comprised of a peptide or polypeptide. In one embodiment a backbone repeat unit of a stabilizing polymer may not be comprised of an amide or polyamide. In one embodiment a backbone repeat unit of a stabilizing polymer may not be comprised of a sugar or carbohydrate. In one embodiment a backbone repeat unit of a stabilizing polymer may not be comprised of a phosphodiester. In on embodiment a backbone repeat unit of a stabilizing polymer may not be comprised of a nucleic acid. In one embodiment a backbone repeat unit of a stabilizing polymer may not be comprised of acrylamide or polyacrylamide.

In one embodiment a stabilizing polymer may be comprised of one or more backbone chains, wherein a backbone chain may be comprised of one or more backbone repeat units.

In one embodiment one or more backbone chains of a stabilizing polymer may be comprised of one or more acrylate or polyacrylate, including, but not limited to, one or more of the following non-limiting examples: acrylate, polyacrylate, poly(methyl acrylate), poly(methacrylate), poly(methyl methacrylate), poly(acrylic acid), poly(methacrylic acid), poly(hydroxyethyl methacrylate), poly(ethyl methacrylate), poly(butyl methacrylate), poly(methoxy ethylacrylate), methyl acrylate, methacrylate, methyl methacrylate, acrylic acid, methacrylic acid, hydroxyethyl methacrylate, ethyl methacrylate, butyl methacrylate, or methoxy ethylacrylate, or combinations thereof.

In one embodiment one or more backbone chains of a stabilizing polymer may be comprised of one or more ester or polyester (such as a carboxylate ester or carbonate ester as non-limiting examples) including, but not limited to, one or more of the following non-limiting examples: carboxylate ester, carbonate ester, polyester, polycarbonate, poly(lactic acid), poly(lactide), poly(glycolide), poly(glycolic acid), poly(lactide-co-glycolide), poly(lactic-co-glycolic acid) polycaprolactone, poly-ε-caprolactone, poly(ethylene glycol)-block-poly(caprolactone), polycaprolactone diol, poly(ethylene glycol)-block-poly(lactic acid), poly(hydroxyalkanoate), poly(hydroxybutyrate), poly[(R)-3-hydroxybutyric acid), poly(3-hydroxybutyrate), poly(3-hydroxybutyrate-co-3-hydroxyvalerate), poly(hydroxy valerate), poly(trimethylene carbonate), poly(ethylene carbonate), poly(propylene carbonate), poly(dioxanone), poly(p-dioxanone), poly(adipic acid), poly(adipate), poly(succinic acid), poly(succinate), poly(fumarate), poly(caprolactone-block-fumarate), polycaprolactone fumarate, poly(ethylene adipate), poly(butylene succinate), poly(ethylene glutarate), poly(glutarate), poly(ethylene glycol diacetate), poly(ethylene azelate), poly(trimethylene glutarate), poly(dioxanone), lactic acid, lactide, glycolide, glycolic acid, caprolactone, ε-caprolactone, hydroxyalkanoate, hydroxybutyrate, 3-hydroxybutyric acid, butyrate, trimethylene carbonate, ethylene carbonate, propylene carbonate, dioxanone, p-dioxanone, adipic acid, adipate, succinic acid, succinate, fumarate, ethylene adipate, butylene succinate, ethylene glutarate, glutarate, ethylene glycol diacetate, ethylene azelate, trimethylene glutarate, or dioxanone, or combinations thereof. As a non-limiting example an ester may be formed by linking one or more backbone repeat unit to an adjacent backbone repeat unit.

In one embodiment one or more backbone chains of a stabilizing polymer may be comprised of one or more ether or polyether, including, but not limited to, one or more of the following non-limiting examples: polyether, poly(ethylene oxide-block-propylene oxide), poly(ether ether ketone), PEEK, poly(ethylene glycol), poly(propylene glycol), poly(propylene oxide glycol), poly(ethylene oxide), poly(ethylene oxide glycol), poly(propylene oxide), poly(tetramethylene glycol), poly(butylene oxide glycol), poly(glycerol), oxetan, poly(tetrahydrofuran), poly(dioxane), poly(dioxanone), ethylene glycol, propylene glycol, propylene oxide glycol, ethylene oxide, ethylene oxide glycol, propylene oxide, tetramethylene glycol, butylene oxide glycol, glycerol, oxetan, tetrahydrofuran, dioxane, or dioxanone, or combinations thereof. As a non-limiting example, an ether may be formed by linking one or more backbone repeat unit to an adjacent backbone repeat unit.

In one embodiment one or more backbone chains of a stabilizing polymer may be comprised of one or more amide or polyamide, including, but not limited to, one or more of the following non-limiting examples: amide, polyamide, nylon, poly(hexamethylene adipamide), nylon 6/6, nylon 6/10, nylon 10/10, nylon 10/12, nylon 11, nylon 12, acrylamide, poly(amino acid), peptide, poly(peptide), poly(aspartic acid), poly(glutamic acid), poly(lysine), poly(histidine), poly(tyrosine), poly(methionine), poly(glycine), poly(proline), poly(cysteine), amino acid, peptide, aspartic acid, glutamic acid, lysine, histidine, tyrosine, methionine, glycine, proline, or cysteine, or combinations thereof.

In one embodiment one or more backbone chains of a stabilizing polymer may be comprised of one or more vinyl or polyvinyl, including but not limited to, one or more of the following non-limiting examples: vinyl, polyvinyl, poly(2-vinylpyridine N-oxide), poly(2-vinyl-1-methylpyridinium), poly(1-methyl-4-vinylpyridinium), poly(4-vinyl-1-methylpyridinium), poly(4-vinyl N-methyl pyridinium), poly(vinylsulfonic acid), poly(vinylsulfonate), poly(N-vinylpyrrolidone-block-vinyl acetate), poly(vinylphosphonic acid), poly(vinylphosphonate), poly(4-vinylphenol), poly(2-vinylpyridine), poly(4-vinylpyridine), polyethylene, polypropylene, polystyrene, poly(styrene sulfonate), poly(vinyl chloride), poly(vinyl acetate), polyacrylonitrile, poly(N-vinylpyrrolidone), poly(vinylpyrrolidone), poly(vinyl sulfate), poly(vinyl sulfonate), poly(vinyl alcohol), poly(diallyldimethylammonium), diallyldimethylammonium, ethylene, propylene, styrene, styrene sulfonate, vinyl chloride, vinyl acetate, acrylonitrile, N-vinylpyrrolidone, vinyl sulfate, vinyl sulfonate, 2-vinylpyridine N-oxide, 2-vinyl-1-methylpyridinium, 1-methyl-4-vinylpyridinium, 4-vinyl-1-methylpyridinium, 4-vinyl N-methyl pyridinium, vinylsulfonic acid, vinylsulfonate, vinyl acetate, vinylphosphonic acid, vinylphosphonate, 4-vinylphenol, 2-vinylpyridine, 4-vinylpyridine, or vinyl alcohol, or combinations thereof.

In one embodiment one or more backbone chains of a stabilizing polymer may be comprised of one or more oxazoline or poly(oxazoline), such as 2-ethyl-2-oxazoline or poly(2-ethyl-2-oxazoline) as a non-limiting example.

In one embodiment one or more backbone chains of a stabilizing polymer may be comprised of one or more acrylamide or polyacrylamide.

In one embodiment one or more backbone chains of a stabilizing polymer may be comprised of one or more diallyldimethylammonium or polydiallyldimethylammonium.

In one embodiment one or more backbone chains of a stabilizing polymer may be comprised of one or more cyclic amide, such as N-vinylpyrrolidone or poly(vinylpyrrolidone).

In one embodiment one or more backbone chains of a stabilizing polymer may be comprised of one or more phosphoester or polyphosphoester, such as an organophosphate or phosphodiester as non-limiting examples.

In one embodiment one or more backbone chains of a stabilizing polymer may be comprised of one or more phosphazene or poly(phosphazene), such as an organophosphazene or poly(organophosphazene).

In one embodiment one or more backbone chains of a stabilizing polymer may be comprised of one or more siloxane or polysiloxane, such as dimethyl siloxane or polydimethylsiloxane as non-limiting examples.

In one embodiment one or more backbone chains of a stabilizing polymer may be comprised of one or more imide or polyimide.

In one embodiment one or more backbone chains of a stabilizing polymer may be comprised of one or more urea or polyurea.

In one embodiment one or more backbone chains of a stabilizing polymer may be comprised of one or more amine or polyamine.

In one embodiment one or more backbone chains of a stabilizing polymer may be comprised of one or more amine or polyamine, wherein an amine or polyamine may comprise a quaternary ammonium cation, such as hexadimethrine as a non-limiting example.

In one embodiment one or more backbone chains of a stabilizing polymer may be comprised of one or more amine or polyamine, wherein an amine or polyamine may comprise a tertiary amine.

In one embodiment one or more backbone chains of a stabilizing polymer may be comprised of one or more isocyanate, such as polyurethane as a non-limiting example.

In one embodiment one or more backbone chains of a stabilizing polymer may comprise one or more combinations of one or more of the following: acrylate, polyacrylate, ester, polyester, ether, polyether, amide, polyamide, vinyl, polyvinyl, oxazoline, poly(oxazoline) acrylamide, polyacrylamide, diallyldimethylammonium, polydiallyldimethylammonium, cyclic amide, poly(vinylpyrrolidone), phosphoester, polyphosphoester, phosphazene, poly(phosphazene), organophosphazene, poly(organophosphazene), siloxane, polysiloxane, imide, polyimide, urea, polyurea, amine, polyamine, quaternary ammonium amine, quaternary ammonium polyamine, tertiary amine, tertiary amine polyamine, isocyanate, or polyurethane or combinations thereof.

In one embodiment one or more blocks of a stabilizing polymer comprised of a block copolymer may be comprised of one or more backbone chain, wherein a backbone chain may be at least part of one or more block of a block copolymer or multiblock polymer. In one embodiment one or more backbone chain of a stabilizing polymer comprised of one or more copolymer (such as a graft copolymer or block copolymer as non-limiting examples) may be comprised of one or more backbone chain, wherein a backbone chain may be at least part of one or more copolymer.

In one embodiment a stabilizing polymer may be comprised of one or more type of backbone chain.

In one embodiment a stabilizing polymer may be comprised of two or more types of backbone chains, wherein the stabilizing polymer may be comprised of a copolymer such as a bipolymer, terpolymer, or quaterpolymer, or higher order copolymer, such as a graft copolymer, a star copolymer, crosslinked copolymer, or a block copolymer, as non-limiting examples.

In one embodiment a stabilizing polymer may be comprised of two or more types of backbone chains, wherein the stabilizing polymer may be comprised of a block copolymer, such as a diblock polymer, triblock polymer, quaterblock polymer, or higher order multiblock polymer, such as a linear block copolymer, a random block copolymer, or an alternating block copolymer, as non-limiting examples.

In one embodiment a stabilizing polymer may comprise one or more combinations of one or more backbone repeat units or one or more backbone chains such as the following non-limiting examples: polyethylene glycol monomethacrylate, poly(N-vinylpyrrolidone-block-vinyl acetate), poly(ethylene oxide-block-propylene oxide), poly(dimethylsiloxane-block-ethylene oxide), poly(ethylene glycol) dimethacrylate, poly(dimethylsiloxane-block-ethylene oxide), poly(ethylene glycol-block-methacrylate), poly(ethylene glycol-block-methyl methacrylate), poly(lactide-co-glycolide), poly(methyl methacrylate-co-vinylpyrrolidone), poly(1-vinylpyrrolidone-co-styrene), poly(2-vinylpyridine-co-styrene), poly(4-vinylpyridine-co-styrene), poly(acrylamide-co-diallyldimethylammonium), or poly(l-vinylpyrrolidone-co-vinyl acetate), as non-limiting examples.

In one embodiment a backbone chain of a stabilizing polymer may not be comprised of an amino acid. In one embodiment a backbone chain of a stabilizing polymer may not be comprised of a peptide or polypeptide. In one embodiment a backbone chain of a stabilizing polymer may not be comprised of an amide or polyamide. In one embodiment a backbone chain of a stabilizing polymer may not be comprised of a sugar, saccharide, starch, or carbohydrate. In one embodiment a backbone chain of a stabilizing polymer may not be comprised of a phosphodiester. In on embodiment a backbone chain of a stabilizing polymer may not be comprised of a nucleic acid. In one embodiment a backbone chain of a stabilizing polymer may not be comprised of acrylamide or polyacrylamide.

In one embodiment a stabilizing polymer may be comprised of single backbone chain. In one embodiment a stabilizing polymer may be comprised of one or more backbone chains, wherein the backbone chains may be the same or different. In other embodiments a stabilizing polymer may be comprised of two or more backbone chains, wherein the backbone chains may be the same or different. In one embodiment a stabilizing polymer may be comprised of one or more type of backbone repeat unit. In other embodiments a stabilizing polymer may be comprised of two or more types backbone repeat units, such as in a copolymer or block copolymer as non-limiting examples.

In one embodiment a stabilizing polymer may be comprised of a homopolymer. In one embodiment a stabilizing polymer may be comprised of copolymer, such as an alternating copolymer, block copolymer, or random copolymer, as non-limiting examples.

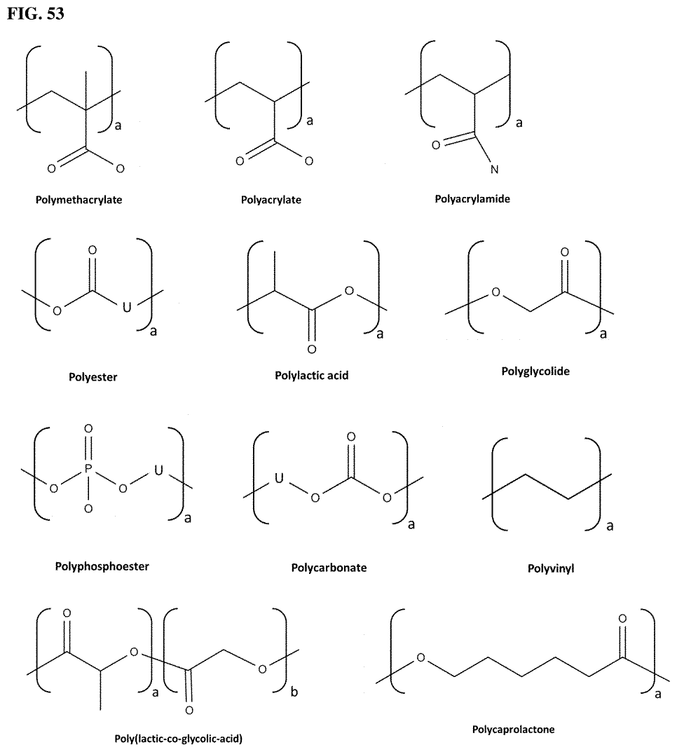

As a non-limiting example, a stabilizing polymer may comprise a backbone chain comprised of one or more backbone repeat units shown in FIG. 53, wherein one or more backbone chain of a stabilizing polymer or one or more backbone repeat unit of a stabilizing polymer may comprise one or more example substances, including but not limited to: polymethacrylate, polyacrylate, polyacrylamide, polyester, polylactic acid, polyglycolide, polyphosphoester, polycarbonate, polyvinyl, poly(lactic-co-glycolic-acid), or polycaprolactone. In the non-limiting example shown in FIG. 53, a and b are integers that may be the same or different and determine the number of backbone repeat units within a backbone chain of a stabilizing polymer, as a non-limiting example a or b may be an integer between about 2-10,000. Furthermore, U is a U group as described herein and may be comprised at least one carbon atom.

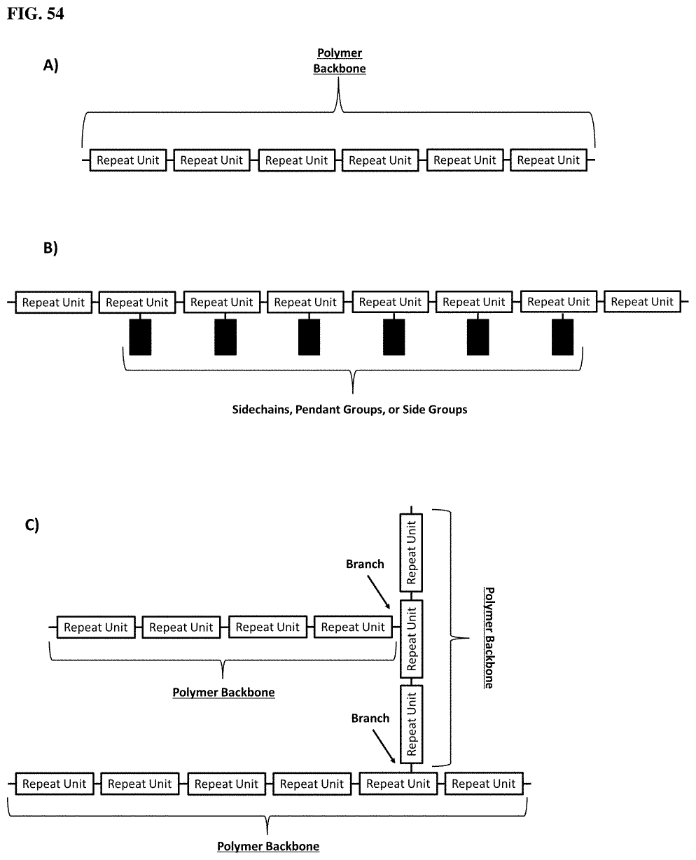

FIG. 54 shows a non-limiting example of a stabilizing polymer. A stabilizing polymer may be comprised of one or more backbone chains comprised of one or more backbone repeat units shown in FIG. 54A. Furthermore, a stabilizing polymer may be comprised of one or more sidechains, pendant groups, or side groups, wherein a backbone chain of a stabilizing polymer or backbone repeat unit of a stabilizing polymer may comprise one or more sidechains, pendant groups, or side groups shown in FIG. 54B. A sidechain, pendant group, or side group may be comprised of one or more stabilizing monomer as described herein. Finally, a stabilizing polymer may also be comprised of one or more branches as shown in FIG. 54C as described herein.

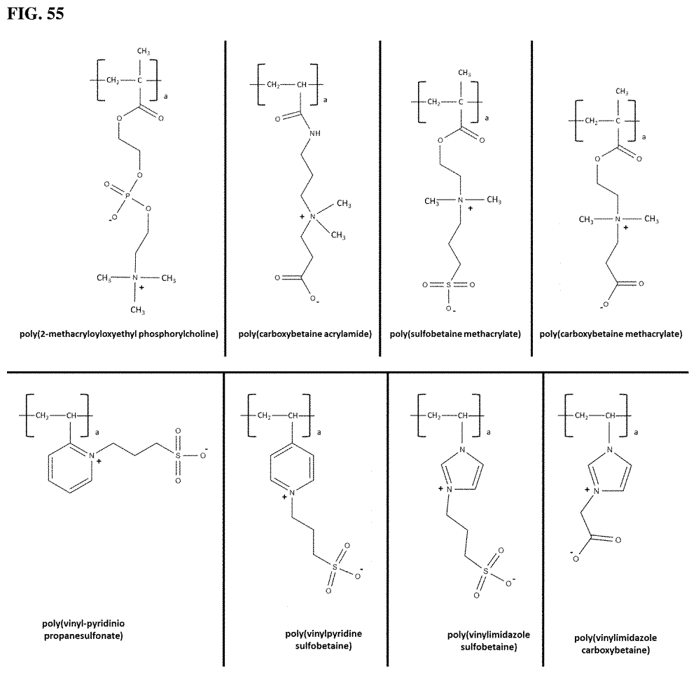

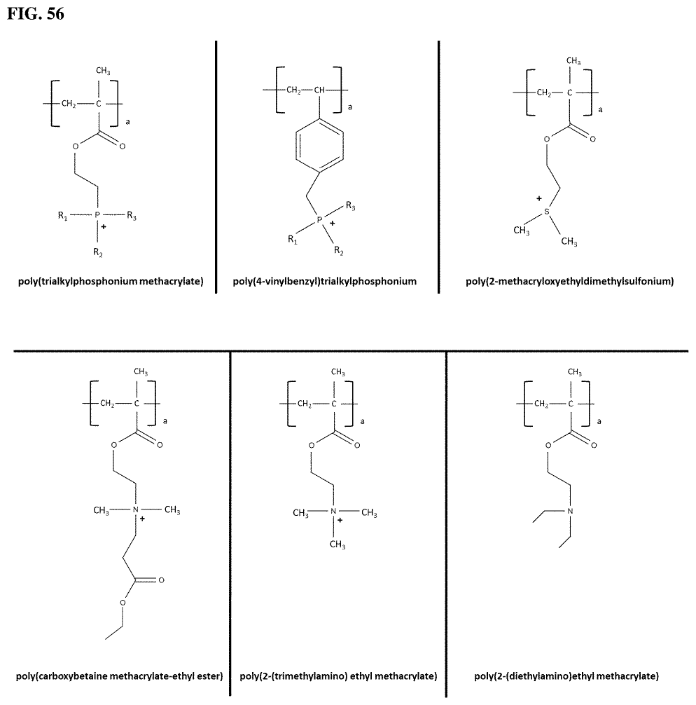

FIG. 55 and FIG. 56 shows non-limiting examples of example stabilizing polymers comprised of one or more stabilizing monomers as described herein, including but not limited to: poly(2-methacryloyloxyethyl phosphorylcholine), poly(carboxybetaine acrylamide), poly(sulfobetaine methacrylate), poly(carboxybetaine methacrylate), poly(vinyl-pyridinio propanesulfonate), poly(vinylpyridine sulfobetaine), poly(vinylimidazole sulfobetaine), poly(vinylimidazole carboxybetaine), poly(trialkylphosphonium methacrylate), poly(4-vinylbenzyl)trialkylphosphonium, poly(2-methacryloxyethyldimethylsulfonium), poly(carboxybetaine methacrylate-ethyl ester), poly(2-(trimethylamino) ethyl methacrylate), poly(2-(diethylamino)ethyl methacrylate).

### Stabilizing Polymer Compositions:

The inventors have discovered that RNA stabilizing substances may comprise stabilizing polymers. The inventors have also discovered that the stability of RNA substances may be enhanced in an RNA storage environment comprising one or more stabilizing polymer. The inventors have also discovered that the stability of RNA substances may be enhanced in an RNA storage environment comprising one or more stabilizing polymer and one or more additional RNA stabilizing substance, such as a betaine containing substance.

The inventors have also discovered that the stability of RNA substances may be enhanced in compositions comprising an RNA substance and a stabilizing polymer. The inventors have also discovered that the stability of RNA substances may be enhanced in compositions comprising an RNA substance, a stabilizing polymer, and one or more additional RNA stabilizing substance, such as a betaine containing substance.

In one embodiment of the present invention, one or more RNA stabilizing substance may comprise a stabilizing polymer.

One embodiment of the present invention may include a composition comprised of one or more RNA substance and one or more stabilizing polymer. Another embodiment of the present invention may include a composition comprised of one or more RNA substance, one or more stabilizing polymer, and one or more additional RNA stabilizing substance, such as a betaine containing substance.

One embodiment of the present invention may include a combination or mixture comprising one or more RNA substance and one or more stabilizing polymer. Another embodiment of the present invention may include a combination or mixture comprising one or more RNA substance, one or more stabilizing polymer, and one or more additional RNA stabilizing substance, such as a betaine containing substance.

Embodiments of the present invention that comprise one or more RNA substance and one or more stabilizing polymer may include combining, such as by mixing, one or more RNA substance with one or more substance that comprises at least one or more stabilizing polymer.

Embodiments of the present invention that comprise one or more RNA substance, one or more stabilizing polymer, and one or more additional RNA stabilizing substance, such as a betaine containing substance, may include combining, such as by mixing, one or more RNA substance with one or more substance that comprises at least one or more stabilizing polymer and one or more substance that comprises at least one or more RNA stabilizing substance, such as a betaine containing substance.

An embodiment of the present invention may include compositions of materials that comprise one or more RNA substance and at least one or more stabilizing polymer. The storage environment that improves the stability of RNA substances may comprise at least one or more vapor, liquid, powder, or solid stabilizing polymer.

An embodiment of the present invention may include compositions of materials that comprise one or more RNA substance, one or more stabilizing polymer, and one or more additional RNA stabilizing substance, such as a betaine containing substance. The storage environment that improves the stability of RNA substances may comprise at least one or more vapor, liquid, powder, or solid stabilizing polymer and may comprise at least one or more vapor, liquid, powder, or solid RNA stabilizing substance, such as a betaine containing substance.

An embodiment of the present invention may include combinations of materials that comprise one or more RNA substance and at least one or more stabilizing polymer. The storage environment that improves the stability of RNA substances may comprise at least one or more vapor, liquid, powder, or solid stabilizing polymer.

An embodiment of the present invention may include combinations of materials that comprise one or more RNA substance, one or more stabilizing polymer, and one or more additional RNA stabilizing substance, such as a betaine containing substance. The storage environment that improves the stability of RNA substances may comprise at least one or more vapor, liquid, powder, or solid stabilizing polymer and may comprise at least one or more vapor, liquid, powder, or solid RNA stabilizing substance, such as a betaine containing substance.

In one embodiment of the present invention a composition comprised of one or more RNA substance and one or more stabilizing polymer, produces a mixture with at least one or more RNA substance and at least one or more stabilizing polymer. In one embodiment of the present invention a composition comprised of one or more RNA substance, one or more stabilizing polymer, and one or more additional RNA stabilizing substance, such as a betaine containing substance, produces a mixture with at least one or more RNA substance, at least one or more stabilizing polymer, and at least one or more additional RNA stabilizing substance, such as a betaine containing substance.

In one embodiment of the present invention a combination comprising one or more RNA substance and one or more stabilizing polymer, produces a mixture with at least one or more RNA substance and at least one or more stabilizing polymer. In one embodiment of the present invention a combination comprising one or more RNA substance, one or more stabilizing polymer, and one or more additional RNA stabilizing substance, such as a betaine containing substance, produces a mixture with at least one or more RNA substance, at least one or more stabilizing polymer, and at least one or more additional RNA stabilizing substance, such as a betaine containing substance.

In one embodiment of the present invention a composition with improved RNA stability may comprise one or more RNA substance and one or more stabilizing polymer. In one embodiment of the present invention a composition with improved RNA stability may comprise one or more RNA substance, one or more stabilizing polymer, and one or more additional RNA stabilizing substance, such as a betaine containing substance.

In one embodiment of the present invention a composition with improved RNA stability may comprise a mixture of one or more RNA substance and one or more stabilizing polymer. In one embodiment of the present invention a composition with improved RNA stability may comprise a mixture of one or more RNA substance, one or more stabilizing polymer, and one or more additional RNA stabilizing substance, such as a betaine containing substance.

In one embodiment of the present invention, each component included in a composition comprising one or more RNA substance and one or more stabilizing polymer, may be stored separately, such as in a kit, or such as individual substances or as mixtures of one or more substance, and then combined later to produce a composition comprising one or more RNA substance and one or more stabilizing polymer.

In one embodiment of the present invention, each component included in a composition comprising one or more RNA substance, one or more stabilizing polymer, and one or more additional RNA stabilizing substance, such as a betaine containing substance, may be stored separately, such as in a kit, or such as individually or as mixtures of one or more substance, and then combined later to produce a composition comprising one or more RNA substance, one or more stabilizing polymer, and one or more additional RNA stabilizing substance, such as a betaine containing substance.

In one embodiment of the present invention a composition comprising one or more RNA substance and one or more stabilizing polymer may be at least partially biocompatible.

In one embodiment of the present invention a composition comprising one or more RNA substance, one or more stabilizing polymer, and one or more additional RNA stabilizing substance, such as a betaine containing substance, may be at least partially biocompatible.

In one embodiment of the present invention a combination comprised of one or more RNA substance and one or more stabilizing polymer may be at least partially biocompatible.

In one embodiment of the present invention a combination comprised of one or more RNA substance, one or more stabilizing polymer, and one or more additional RNA stabilizing substance, such as a betaine containing substance, may be at least partially biocompatible.

Embodiments of the present invention comprising at least one or more stabilizing polymer may include one or more forms of the substance. These forms may include, but are not limited to, one more counterions, ionic forms, conjugate bases, conjugate acids, protonated or deprotonated forms, hydrated or dehydrated forms, isomers, structural isomers, stereo isomers, chiral forms, salts, or combinations thereof.

In one embodiment a stabilizing polymer may comprise one or more of the following formulas:

-[(A-R_{LA}-M_{A})ₐ]-, or -[(A)ₐ]-, or

-{[(A-R_{LA}-M_{A})-(B—R_{LB}-M_{B})]_{c}}-, or -[(A-R_{LA}-M_{A})ₐ]-[(B—R_{LB}-M_{B})_{b}]-, or

-{[(A)-(B)]_{c}}—, or -[(A)ₐ]-[(B)_{b}]—, or

-{[(A-R_{LA}-MA)-(B)]_{c}}—, or -[(A-R_{LA}-M_{A})ₐ]-[(B)_{b}]—

Such as the following:

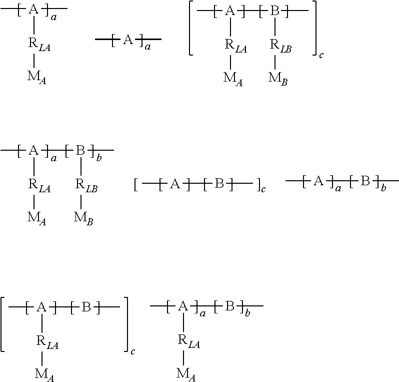

Wherein A and B may be a backbone repeat unit or backbone chain comprised of one or more of the following:

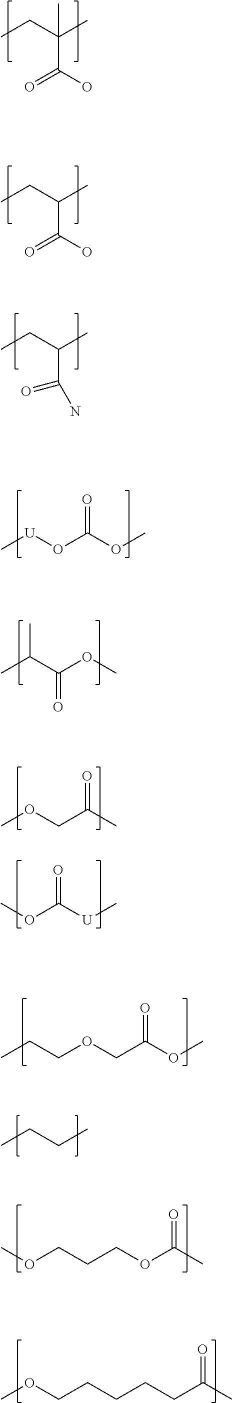

Where A and B may also comprise polymethacrylate, polyacrylate, polyacrylamide, polycarbonate, polylactic acid, polyglycolide, polyester, polydioxanone, polyvinyl, poly(trimethylene carbonate, or polycaprolactone. Furthermore, U is a U group that may be comprised of at least one carbon, as described herein. Meanwhile, a, b, and c may be an integer determining the number of repeated units, such as the number of backbone repeat units within a backbone chain of a stabilizing polymer as a non-limiting. Furthermore as a non-limiting example a, b, or c may be an integer between about 2-10,000.

Furthermore, A and B may be a backbone repeat unit or backbone chain comprised of one or more of the following: acrylate, polyacrylate, ester, polyester, ether, polyether, amide, polyamide, vinyl, polyvinyl, oxazoline, poly(oxazoline) acrylamide, polyacrylamide, diallyldimethylammonium, polydiallyldimethylammonium, cyclic amide, poly(vinylpyrrolidone), phosphoester, polyphosphoester, phosphazene, poly(phosphazene), organophosphazene, poly(organophosphazene), siloxane, polysiloxane, imide, polyimide, urea, polyurea, amine, polyamine, quaternary ammonium amine, quaternary ammonium polyamine, tertiary amine, tertiary amine polyamine, isocyanate, or polyurethane or combinations thereof.

Meanwhile, M is a stabilizing monomer and may be covalently bonded to a stabilizing polymer by a linker represented by R_{L}, wherein R_{L} is an R group of at least one stabilizing monomer covalently bonded to at least part of a stabilizing polymer.

In one embodiment a U group may be comprised of at least one carbon atom. Wherein, U may be comprised of a C1-35 alkyl, alkenyl, alkynyl, aryl, or aralkyl group, each of which may contain up to 20 heteroatoms. In another embodiment a U group may be comprised of C1-30 alkyl, alkenyl, alkynyl, aryl, or aralkyl group, each of which may contain up to 16 heteroatoms. In another embodiment a U group may be comprised of C1-24 alkyl, alkenyl, alkynyl, aryl, or aralkyl group, each of which may contain up to 12 heteroatoms. In another embodiment a U group may be comprised of C1-20 alkyl, alkenyl, alkynyl, aryl, or aralkyl group, each of which may contain up to 10 heteroatoms. In another embodiment a U group may be comprised of C1-16 alkyl, alkenyl, alkynyl, aryl, or aralkyl group, each of which may contain up to 8 heteroatoms. In another embodiment a U group may be comprised of C1-12 alkyl, alkenyl, alkynyl, aryl, or aralkyl group, each of which may contain up to 6 heteroatoms. In another embodiment a U group may be comprised of C1-12 alkyl, alkenyl, alkynyl, aryl, or aralkyl group, each of which may contain up to 4 heteroatoms. In another embodiment a U group may be comprised of C1-8 alkyl, alkenyl, alkynyl, aryl, or aralkyl group, each of which may contain up to 4 heteroatoms. In another embodiment a U group may be comprised of C1-8 alkyl, alkenyl, alkynyl, aryl, or aralkyl group, each of which may contain up to 3 heteroatoms. In another embodiment a U group may be comprised of C1-6 alkyl, alkenyl, alkynyl, aryl, or aralkyl group, each of which may contain up to 3 heteroatoms. In another embodiment a U group may be comprised of C1-6 alkyl, alkenyl, alkynyl, aryl, or aralkyl group, each of which may contain up to 2 heteroatoms. In another embodiment a U group may be comprised of C1-4 alkyl, alkenyl, alkynyl, aryl, or aralkyl group, each of which may contain up to 2 heteroatoms. In another embodiment a U group may be comprised of C1-4 alkyl, alkenyl, alkynyl, aryl, or aralkyl group, each of which may contain up to 1 heteroatom.

In one embodiment a U group may be aliphatic.

In one embodiment a, b, or c may be an integer between about 2-10,000, or between about 2-5,000, or between about 2-1,000, or between about 2-800, or between about 2-600, or between about 2-400, or between about 2-200, or between about 2-100, or between about 2-50, or between about 2-40, or between about 2-30, or between about 2-20, or between about 2-10. In one embodiment a, b, or c may be an integer between about 5-10,000, or between about 5-5,000, or between about 5-1,000, or between about 5-800, or between about 5-600, or between about 5-400, or between about 5-200, or between about 5-100, or between about 5-50, or between about 5-40, or between about 5-30, or between about 5-20, or between about 5-10. In one embodiment a, b, or c may be an integer between about 10-10,000, or between about 10-5,000, or between about 10-1,000, or between about 10-800, or between about 10-600, or between about 10-400, or between about 10-200, or between about 10-100, or between about 10-50, or between about 10-40, or between about 10-30, or between about 10-20. In one embodiment a, b, or c may be an integer greater than about 2, or greater than about 5, or greater than about 10, or greater the 20, or greater than about 30, or greater than about 40, or greater than about 50, or greater than about 100, or greater than about 500, or greater than about 1,000. In one embodiment a, b, or c may be an integer up to about 20, or up to about 50, or up to about 100, or up to about 500, or up to about 1,000, or up to about 2,000, or up to about 5,000, or up to about 10,000, or up to about 50,000, or up to about 100,000.

In one embodiment R_{L} may comprise one or more R group of one or more stabilizing monomer covalently bonded to at least part of a stabilizing polymer. In one embodiment R_{L} may comprise one or more R group of one or more stabilizing monomer covalently bonded to at least part of one or more backbone chain of a stabilizing polymer. In one embodiment R_{L} may comprise one or more R group of one or more stabilizing monomer covalently bonded to at least part of one or more backbone repeat unit of a stabilizing polymer. In one embodiment R_{L} may comprise one or more R group of one or more stabilizing monomer covalently bonded to at least part of one or more stabilizing polymer sidechain, pendant group, or side group covalently bonded to at least part of one or more backbone chain or one or more backbone repeat unit of a stabilizing polymer.

In one embodiment a stabilizing polymer may be comprised of a copolymer, such as a bipolymer, terpolymer, or quaterpolymer, or higher order copolymer. In one embodiment a stabilizing polymer may be comprised of one or more the following types of copolymers, including, but not limited to: a linear copolymer, a random copolymer, an alternating copolymer, a statistical copolymer, a gradient copolymer, a periodic copolymer, a sequential copolymer, a block copolymer, a graft copolymer, a crosslinked copolymer, or a star copolymer, or combinations thereof.

In one embodiment a stabilizing polymer may be comprised of a block copolymer, wherein a stabilizing polymer may be comprised of a diblock polymer, triblock polymer, quaterblock polymer, or higher order multiblock polymer.

In one embodiment a stabilizing polymer may be comprised of a linear block copolymer. In one embodiment a stabilizing polymer may be comprised of a random block copolymer. In one embodiment a stabilizing polymer may be comprised of an alternating block copolymer.

In one embodiment A and B may be interchangeable or alternating blocks or interchangeable or alternating units of a copolymer or a block copolymer wherein A and B may comprise the same or different monomers, or backbone repeat units, or backbone chains. In one embodiment A and B may have the same number of repeated units or a different number of repeated units, such that a or b may be the same or different integers.

Furthermore, the above formulas may comprise one or more additional blocks or units such as a C unit, D unit, or E unit as nonlimiting examples, such that in addition to A and B, a stabilizing polymer may also comprise one or more additional blocks or units of a copolymer or coblock polymer, such as a C unit, or D unit, or E unit as non-limiting examples. Wherein one or more additional units or blocks may be substituted for or added to one or more of the above formulas comprising an A unit and a B unit, such that a stabilizing polymer may also comprise one or more C unit or D unit or E unit as non-limiting examples. In one embodiment an A unit or a B unit may be combined or interchangeable with one or more other unit, such as a C unit, or D unit, or E unit as non-limiting examples.

In a non-limiting example, a stabilizing polymer may comprise the formula -[(A-R_{LA}-M_{A})ₐ]-[(B—R_{LB}-M_{B})_{b}]-[(D-R_{LD}-M_{D})_{d}]- or the formula -[(A-R_{LA}-M_{A})ₐ]-[(B—R_{LB}-M_{B})_{b}]-[(D-R_{LD}-M_{D})_{d}]-[(E-R_{LE}-M_{E})ₑ]- wherein D or E may be one or more additional unit or block of a stabilizing polymer, wherein a stabilizing polymer may comprise a terpolymer, or quaterpolymer, or higher order copolymer, or a triblock polymer, quaterblock polymer, or higher order multiblock polymer. Furthermore, a, b, d, or e may be independently selected integers determining the number of repeated units and may be the same or different.

In embodiment one or more backbone repeat unit of a stabilizing polymer may not comprise one or more stabilizing monomers. In one embodiment one or more backbone repeat unit of a stabilizing polymer may not comprise one or more sidechain, pendant group, or side group.

In embodiment one or more stabilizing monomers may be absent from one or more backbone repeat unit of a stabilizing polymer. In one embodiment one or more sidechain, pendant group, or side group may be absent from one or more backbone repeat unit of a stabilizing polymer.

In embodiment one or more backbone chain of a stabilizing polymer may not comprise one or more stabilizing monomers. In one embodiment one or more backbone chain of a stabilizing polymer may not comprise one or more sidechain, pendant group, or side group.

In embodiment one or more stabilizing monomers may be absent from one or more backbone chain of a stabilizing polymer. In one embodiment one or more sidechain, pendant group, or side group may be absent from one or more backbone chain of a stabilizing polymer.

In a non-limiting example, a stabilizing polymer may comprise the formula -[(A-R_{LA}-M_{A})ₐ]-[(B)_{b}]-[(D-R_{LD}-M_{D})_{d}]- or the formula -[(A-R_{LA}-M_{A})ₐ]-[(B)_{b}]-[(D-R_{LD}-M_{D})_{d}]-[(E)ₑ]- wherein a B unit or E unit does not comprise a stabilizing monomer and D or E may be one or more additional unit or block of a stabilizing polymer, wherein a stabilizing polymer may comprise a terpolymer, or quaterpolymer, or higher order copolymer, or a triblock polymer, quaterblock polymer, or higher order multiblock polymer. Furthermore, a, b, d, or e may be independently selected integers determining the number of repeated units and may be the same or different.

Non-limiting examples of stabilizing polymers of the present invention that may be suitable for use, may include, but are not limited to, those described as ammonium and/or phosphonium-containing polymers and/or block copolymers, ammonium- and phosphonium containing styrenic homopolymers, phosphonium-comprising polymers, phosphonium containing co-polymers, block co-polymers, stabilization blocks, complexation blocks, or endosomolytic blocks in WO Patent Application Pub. No. WO 2012/174543 A2, incorporated herein by reference. The above reference also provides synthesis details for synthesizing one or more non-limiting examples of one or more stabilizing polymer as described herein.

Non-limiting examples of stabilizing polymers of the present invention that may be suitable for use, may include, but are not limited to, those described as sulfonium homopolymers, sulfonium diblock copolymers, or sulfonium-containing diblock copolymers in “S. T. Hemp, M. H. Allen, A. E. Smith, T. E. Long, Synthesis and Properties of Sulfonium Polyelectrolytes for Biological Applications, ACS Macro Lett. 2 (2013) 731-735. https://doi.org/10.1021/mz4002172.” incorporated herein by reference. The above reference also provides synthesis details for synthesizing non-limiting examples of one or more stabilizing polymer as described herein.

Non-limiting examples of stabilizing polymers of the present invention that may be suitable for use, may include, but are not limited to, those described as poly(carboxybetaine)s, cationic polymers, or cationic polyelectrolytes in “D.-J. Liaw, C.-C. Huang, W.-F. Lee, J. Borbely, E.-T. Kang, Synthesis and characteristics of the poly(carboxybetaine)s and the corresponding cationic polymers, J. Polym. Sci. A Polym. Chem. 35 (1997) 3527-3536. https://doi.org/10.1002/(SICI)1099-0518(19971130)35:16<3527::AID-POLA119>3.0.CO:2-H.”, incorporated herein by reference. The above reference also provides synthesis details for synthesizing non-limiting examples of one or more stabilizing polymer as described herein.

Non-limiting examples of stabilizing polymers of the present invention that may be suitable for use, may include, but are not limited to, those described as poly(sulfobetaine)s or cationic polymers in “W.-F. Lee, C.-C. Tsai, Synthesis and solubility of the poly(sulfobetaine)s and the corresponding cationic polymers: 1. Synthesis and characterization of sulfobetaines and the corresponding cationic monomers by nuclear magnetic resonance spectra, Polymer. 35 (1994) 2210-2217. https://doi.org/10.1016/0032-3861(94)90253-4.”, incorporated herein by reference. The above reference also provides synthesis details for synthesizing non-limiting examples of one or more stabilizing polymer as described herein.

Non-limiting examples of stabilizing polymers of the present invention that may be suitable for use, may include, but are not limited to, those described as poly(sulfobetaine methacrylate) (PSBMA) in “R. Lalani, L. Liu, Synthesis, characterization, and electrospinning of zwitterionic poly(sulfobetaine methacrylate), Polymer. 52 (2011) 5344-5354. https://doi.org/10.1016/j.polymer.2011.09.015.”, incorporated herein by reference. The above reference also provides synthesis details for synthesizing non-limiting examples of one or more stabilizing polymer as described herein.

Non-limiting examples of stabilizing polymers of the present invention that may be suitable for use, may include, but are not limited to, those described as zwitterionic polymers, polyzwitterions, polybetaines, polymeric zwitterions, polycarboxybetaines, polysulfobetaines, polymeric phosphobetaines, or poly(phosphobetaine)s in “A. Laschewsky, Structures and Synthesis of Zwitterionic Polymers, Polymers. 6 (2014) 1544-1601. https://doi.org/10/3390/polym6051544.”, incorporated herein by reference. The above reference also provides synthesis details for synthesizing non-limiting examples of one or more stabilizing polymer as described herein.

Non-limiting examples of stabilizing polymers of the present invention that may be suitable for use, may include, but are not limited to, those described as carboxybetaine functionalized polysiloxanes, zwitterionic polymers, carboxybetaine functionalized polymer, or PDMS-g-CB in “L. Cheng, Q. Liu, Y. Lei, Y. Lin, A. Zhang, The synthesis and characterization of carboxybetaine functionalized polysiloxanes for the preparation of anti-fouling surfaces, RSC Adv. 4 (2014) 54372-54381. https://doi.org/10.1039/C4RA09171J.”, incorporated herein by reference. The above reference also provides synthesis details for synthesizing non-limiting examples of one or more stabilizing polymer as described herein.

Non-limiting examples of stabilizing polymers of the present invention that may be suitable for use, may include, but are not limited to, those described as polycarboxybetaine esters, cationic polycarboxybetaine esters, carboxybetaine ester polymers, polycarboxybetaines, or zwitterionic polycarboxybetaines in “Z. Zhang, G. Cheng, L. R. Carr, H. Vaisocherová, S. Chen, S. Jiang, The hydrolysis of cationic polycarboxybetaine esters to zwitterionic polycarboxybetaines with controlled properties, Biomaterials. 29 (2008) 4719-4725. https://doi.org/10.1016/j.biomaterials.2008.08.030.”, incorporated herein by reference. The above reference also provides synthesis details for synthesizing non-limiting examples of one or more stabilizing polymer as described herein.

Non-limiting examples of stabilizing polymers of the present invention that may be suitable for use, may include, but are not limited to, those described as CBMA-ethyl ester polymers, pCBMA-ethyl ester, pCBMA-EE, or cationic CBMA ester polymer in “L. R. Carr, S. Jiang, Mediating high levels of gene transfer without cytotoxicity via hydrolytic cationic ester polymers, Biomaterials. 31 (2010) 4186-4193. https://doi.org/10.1016/j.biomaterials.2010.01.110.”, incorporated herein by reference. The above reference also provides synthesis details for synthesizing non-limiting examples of one or more stabilizing polymer as described herein.

A non-limiting example of a stabilizing polymer comprised of the formula -[(A-R_{LA}-M_{A})ₐ]- is poly(2-methacryloyloxyethyl phosphorylcholine) (PMPC), wherein A is a polymethacrylate backbone repeat unit, M_{A} is a stabilizing monomer comprised of phosphorylcholine, and R_{LA} is an R group of a stabilizing monomer comprised of an ethyl group covalently linking a phosphorylcholine monomer to a polymethacrylate backbone.

A non-limiting example of a stabilizing polymer comprised of the formula -[(A)ₐ]- is polydiallyldimethylammonium chloride (PDADMAC), wherein A is a polydiallyldimethylammonium backbone repeat unit.

A non-limiting example of a stabilizing polymer comprised of the formula -[(A-R_{LA}-M_{A})ₐ]-[(B)_{b}]- is poly(sulfobetaine methacrylate)-block-poly(ethylene glycol) (PSBMA-PEG), wherein A is a polymethacrylate backbone repeat unit, MA is a stabilizing monomer comprised of NDSB-195, and R_{LA} is an R group of the stabilizing monomer comprised of an ethyl group covalently linking an NDSB-195 monomer to a polymethacrylate backbone. Furthermore, B is a polyethylene glycol backbone repeat unit, wherein A and B are connected to form a block copolymer, such that block A is comprised of poly(sulfobetaine methacrylate) and block B is comprised of poly(ethylene glycol).

A non-limiting example of a stabilizing polymer comprised of the formula -[(A-R_{LA}-M_{A})ₐ]-[(B)_{b}]- is poly[(2-ethyldimethylammonioethyl methacrylate)-co-(1-vinylpyrrolidone)] (Polyquat 11) wherein A is a polymethacrylate backbone repeat unit, M_{A} is a stabilizing monomer comprised of a quaternary ammonium covalently bonded to two methyl groups and two ethyl groups, and R_{LA} is an R group of the stabilizing monomer comprised of an ethyl group covalently linking a quaternary ammonium monomer to a polymethacrylate backbone. Furthermore, B is a poly(vinylpyrrolidone) backbone repeat unit, wherein A and B are connected to form a block copolymer, such that block A is comprised of poly(ethyldimethylammonioethyl methacrylate) and block B is comprised of poly(vinylpyrrolidone).

A non-limiting example of a stabilizing polymer comprised of the formula -{[(A-R_{LA}-M_{A})-(B—R_{LB}-M_{B})]_{c}}- is poly[(carboxybetaine methacrylate-ethyl ester)-(2-methacryloyloxyethyl phosphorylcholine)], wherein A is a polymethacrylate backbone repeat unit, M_{A} is a stabilizing monomer comprised of quaternary ammonium covalently with two methyl groups and two ethyl groups where one ethyl group is bonded to a carboxylate ester wherein the carboxylate ester shares one ethyl group with the quaternary ammonium and is also bonded to an additional ethyl group, while R_{LA} is an R group of the stabilizing monomer comprised of an ethyl group covalently linking the quaternary ammonium ester to the polymethacrylate backbone. Furthermore, B is a polymethacrylate backbone repeat unit, M_{B} is a stabilizing monomer comprised of phosphorylcholine, and R_{LB} is an R group of a stabilizing monomer comprised of an ethyl group covalently linking a phosphorylcholine monomer to a polymethacrylate backbone. Finally, the A unit and the B unit may be part of a larger repeating unit, wherein c determines the number of repeated units such that the stabilizing polymer is comprised of an alternating copolymer, with repeating carboxybetaine ethyl ester/2-methacryloyloxyethyl phosphorylcholine units.

A non-limiting example of a stabilizing polymer, comprised of one or more stabilizing monomer, that may be used is poly(2-methacryloyloxyethyl phosphorylcholine) (also known as PMPC), such as PMPC (˜9 kDa) as a non-limiting example, wherein PMPC may be substituted for or used in combination with one or more stabilizing polymer as described herein. One embodiment of the present invention may include combinations of substances comprising PMPC and another embodiment may also comprise at least one RNA substance. Other embodiments may include combinations of substances comprising PMPC wherein the concentration of PMPC may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising PMPC wherein the ratio of PMPC to one or more RNA substance may include one or more ratio in the stabilizing polymer N/P ratio list, or in the stabilizing polymer N/P ratio range list, or in the stabilizing polymer C/P ratio list, or in the stabilizing polymer C/P ratio range list. Other embodiments may include combinations of substances comprising PMPC wherein the concentration of PMPC may be one or more concentration in the stabilizing substance weight percent concentration list or in the stabilizing substance molar concentration range list or in the stabilizing substance molar concentration list.

Embodiments of compositions comprising one or more RNA stabilizing substance, such as a stabilizing polymer of which PMPC is a non-limiting example, may have different molar concentrations. For conciseness, the following list of molar concentrations with PMPC as a non-limiting example stabilizing polymer, is herein referred to as the stabilizing polymer molar concentration range list.

In one embodiment the concentration of PMPC may be between about 1 nM-1M. In one embodiment the concentration of PMPC may be between about 1 nM-500 mM. In one embodiment the concentration of PMPC may be between about 1 nM-250 mM. In one embodiment the concentration of PMPC may be between about 1 nM-100 mM. In one embodiment the concentration of PMPC may be between about 1 nM-50 mM. In one embodiment the concentration of PMPC may be between about 1 nM-25 mM. In one embodiment the concentration of PMPC may be between about 1 nM-10 mM. In one embodiment the concentration of PMPC may be between about 1 nM-5 mM. In one embodiment the concentration of PMPC may be between about 1 nM-2.5 mM. In one embodiment the concentration of PMPC may be between about 1 nM-1 mM. In one embodiment the concentration of PMPC may be between about 1 nM-500 μM. In one embodiment the concentration of PMPC may be between about 1 nM-250 μM. In one embodiment the concentration of PMPC may be between about 1 nM-100 μM. In one embodiment the concentration of PMPC may be between about 1 nM-50 μM. In one embodiment the concentration of PMPC may be between about 1 nM-25 μM. In one embodiment the concentration of PMPC may be between about 1 nM-10 μM. In one embodiment the concentration of PMPC may be between about 1 μM-1M. In one embodiment the concentration of PMPC may be between about 1 μM-500 mM. In one embodiment the concentration of PMPC may be between about 1 μM-250 mM. In one embodiment the concentration of PMPC may be between about 1 μM-100 mM. In one embodiment the concentration of PMPC may be between about 1 μM-50 mM. In one embodiment the concentration of PMPC may be between about 1 μM-25 mM. In one embodiment the concentration of PMPC may be between about 1 μM-10 mM. In one embodiment the concentration of PMPC may be between about 1 μM-5 mM. In one embodiment the concentration of PMPC may be between about 1 μM-2.5 mM. In one embodiment the concentration of PMPC may be between about 1 μM-1 mM. In one embodiment the concentration of PMPC may be between about 1 μM-500 μM. In one embodiment the concentration of PMPC may be between about 1 μM-250 μM. In one embodiment the concentration of PMPC may be between about 1 μM-100 μM. In one embodiment the concentration of PMPC may be between about 1 μM-50 μM. In one embodiment the concentration of PMPC may be between about 1 μM-25 μM. In one embodiment the concentration of PMPC may be between about 1 μM-10 μM. In one embodiment the concentration of PMPC may be between about 10 μM-1M. In one embodiment the concentration of PMPC may be between about 10 μM-500 mM. In one embodiment the concentration of PMPC may be between about 10 μM-250 mM. In one embodiment the concentration of PMPC may be between about 10 μM-100 mM. In one embodiment the concentration of PMPC may be between about 10 μM-50 mM. In one embodiment the concentration of PMPC may be between about 10 μM-25 mM. In one embodiment the concentration of PMPC may be between about 10 μM-10 mM. In one embodiment the concentration of PMPC may be between about 10 μM-5 mM. In one embodiment the concentration of PMPC may be between about 10 μM-2.5 mM. In one embodiment the concentration of PMPC may be between about 10 μM-1 mM. In one embodiment the concentration of PMPC may be between about 10 μM-500 μM. In one embodiment the concentration of PMPC may be between about 10 μM-250 μM. In one embodiment the concentration of PMPC may be between about 10 μM-100 μM. In one embodiment the concentration of PMPC may be between about 10 μM-50 μM. In one embodiment the concentration of PMPC may be between about 10 μM-25 μM.

Embodiments of compositions comprising one or more RNA stabilizing substance, such as a stabilizing polymer of which PMPC is a non-limiting example, may have different molar concentrations. For conciseness, the following list of molar concentrations with PMPC as a non-limiting example stabilizing polymer, is herein referred to as the stabilizing polymer molar concentration list.

In one embodiment the concentration of PMPC may be at least 1 nM or more. In one embodiment the concentration of PMPC may be at least 10 nM or more. In one embodiment the concentration of PMPC may be at least 100 nM or more. In one embodiment the concentration of PMPC may be at least 1 μM or more. In one embodiment the concentration of PMPC may be at least 10 μM or more. In one embodiment the concentration of PMPC may be at least 100 μM or more. In one embodiment the concentration of PMPC may be at least 1 mM or more. In one embodiment the concentration of PMPC may be at least 10 mM or more. In one embodiment the concentration of PMPC may be at least 50 mM or more. In one embodiment the concentration of PMPC may be at least 100 mM or more. In one embodiment the concentration of PMPC may be at least 150 mM or more. In one embodiment the concentration of PMPC may be at least 200 mM or more. In one embodiment the concentration of PMPC may be at least 250 mM or more. In one embodiment the concentration of PMPC may be at least 300 mM or more. In one embodiment the concentration of PMPC may be at least 350 mM or more. In one embodiment the concentration of PMPC may be at least 400 mM or more. In one embodiment the concentration of PMPC may be at least 500 mM or more. In one embodiment the concentration of PMPC may be at least 600 mM or more. In one embodiment the concentration of PMPC may be at least 700 mM or more. In one embodiment the concentration of PMPC may be at least 800 mM or more. In one embodiment the concentration of PMPC may be at least 1M or more. In one embodiment the concentration of PMPC may be at least 1.5M or more. In one embodiment the concentration of PMPC may be at least 2M or more. In one embodiment the concentration of PMPC may be at least 3M or more.

Embodiments of compositions comprising one or more RNA stabilizing substance, such as a stabilizing polymer of which PMPC is a non-limiting example, may have different milligram concentrations. For conciseness, the following list of milligram concentrations with PMPC as a non-limiting example stabilizing polymer, is herein referred to as the stabilizing polymer milligram concentration range list.

In one embodiment the concentration of PMPC may be between about 0.1 ng-1 g per mL. In one embodiment the concentration of PMPC may be between about 0.1 ng-500 mg per mL. In one embodiment the concentration of PMPC may be between about 0.1 ng-250 mg per mL. In one embodiment the concentration of PMPC may be between about 0.1 ng-100 mg per mL. In one embodiment the concentration of PMPC may be between about 0.1 ng-50 mg per mL. In one embodiment the concentration of PMPC may be between about 0.1 ng-25 mg per mL. In one embodiment the concentration of PMPC may be between about 0.1 ng-10 mg per mL. In one embodiment the concentration of PMPC may be between about 0.1 ng-5 mg per mL. In one embodiment the concentration of PMPC may be between about 0.1 ng-2.5 mg per mL. In one embodiment the concentration of PMPC may be between about 0.1 ng-1 mg per mL. In one embodiment the concentration of PMPC may be between about 0.1 ng-500 μg per mL. In one embodiment the concentration of PMPC may be between about 0.1 ng-250 μg per mL. In one embodiment the concentration of PMPC may be between about 0.1 ng-100 μg per mL. In one embodiment the concentration of PMPC may be between about 1 ng-1 g per mL. In one embodiment the concentration of PMPC may be between about 1 ng-500 mg per mL. In one embodiment the concentration of PMPC may be between about 1 ng-250 mg per mL. In one embodiment the concentration of PMPC may be between about 1 ng-100 mg per mL. In one embodiment the concentration of PMPC may be between about 1 ng-50 mg per mL. In one embodiment the concentration of PMPC may be between about 1 ng-25 mg per mL. In one embodiment the concentration of PMPC may be between about 1 ng-10 mg per mL. In one embodiment the concentration of PMPC may be between about 1 ng-5 mg per mL. In one embodiment the concentration of PMPC may be between about 1 ng-2.5 mg per mL. In one embodiment the concentration of PMPC may be between about 1 ng-1 mg per mL. In one embodiment the concentration of PMPC may be between about 1 ng-500 μg per mL. In one embodiment the concentration of PMPC may be between about 1 ng-250 μg per mL. In one embodiment the concentration of PMPC may be between about 1 ng-100 μg per mL. In one embodiment the concentration of PMPC may be between about 10 ng-1 g per mL. In one embodiment the concentration of PMPC may be between about 10 ng-500 mg per mL. In one embodiment the concentration of PMPC may be between about 10 ng-250 mg per mL. In one embodiment the concentration of PMPC may be between about 10 ng-100 mg per mL. In one embodiment the concentration of PMPC may be between about 10 ng-50 mg per mL. In one embodiment the concentration of PMPC may be between about 10 ng-25 mg per mL. In one embodiment the concentration of PMPC may be between about 10 ng-10 mg per mL. In one embodiment the concentration of PMPC may be between about 10 ng-5 mg per mL. In one embodiment the concentration of PMPC may be between about 10 ng-2.5 mg per mL. In one embodiment the concentration of PMPC may be between about 10 ng-1 mg per mL. In one embodiment the concentration of PMPC may be between about 10 ng-500 μg per mL. In one embodiment the concentration of PMPC may be between about 10 ng-250 μg per mL. In one embodiment the concentration of PMPC may be between about 10 ng-100 μg per mL. In one embodiment the concentration of PMPC may be between about 100 ng-1 g per mL. In one embodiment the concentration of PMPC may be between about 100 ng-500 mg per mL. In one embodiment the concentration of PMPC may be between about 100 ng-250 mg per mL. In one embodiment the concentration of PMPC may be between about 100 ng-100 mg per mL. In one embodiment the concentration of PMPC may be between about 100 ng-50 mg per mL. In one embodiment the concentration of PMPC may be between about 100 ng-25 mg per mL. In one embodiment the concentration of PMPC may be between about 100 ng-10 mg per mL. In one embodiment the concentration of PMPC may be between about 100 ng-5 mg per mL. In one embodiment the concentration of PMPC may be between about 100 ng-2.5 mg per mL. In one embodiment the concentration of PMPC may be between about 100 ng-1 mg per mL. In one embodiment the concentration of PMPC may be between about 100 ng-500 μg per mL. In one embodiment the concentration of PMPC may be between about 100 ng-250 μg per mL. In one embodiment the concentration of PMPC may be between about 100 ng-100 μg per mL. In one embodiment the concentration of PMPC may be between about 1 μg-1 g per mL. In one embodiment the concentration of PMPC may be between about 1 μg-500 mg per mL. In one embodiment the concentration of PMPC may be between about 1 μg-250 mg per mL. In one embodiment the concentration of PMPC may be between about 1 μg-100 mg per mL. In one embodiment the concentration of PMPC may be between about 1 μg-50 mg per mL. In one embodiment the concentration of PMPC may be between about 1 μg-25 mg per mL. In one embodiment the concentration of PMPC may be between about 1 μg-10 mg per mL. In one embodiment the concentration of PMPC may be between about 1 μg-5 mg per mL. In one embodiment the concentration of PMPC may be between about 1 μg-2.5 mg per mL. In one embodiment the concentration of PMPC may be between about 1 μg-1 mg per mL. In one embodiment the concentration of PMPC may be between about 1 μg-500 μg per mL. In one embodiment the concentration of PMPC may be between about 1 μg-250 μg per mL. In one embodiment the concentration of PMPC may be between about 1 μg-100 μg per mL. In one embodiment the concentration of PMPC may be between about 10 μg-1 g per mL. In one embodiment the concentration of PMPC may be between about 10 μg-500 mg per mL. In one embodiment the concentration of PMPC may be between about 10 μg-250 mg per mL. In one embodiment the concentration of PMPC may be between about 10 μg-100 mg per mL. In one embodiment the concentration of PMPC may be between about 10 μg-50 mg per mL. In one embodiment the concentration of PMPC may be between about 10 μg-25 mg per mL. In one embodiment the concentration of PMPC may be between about 10 μg-10 mg per mL. In one embodiment the concentration of PMPC may be between about 10 μg-5 mg per mL. In one embodiment the concentration of PMPC may be between about 10 μg-2.5 mg per mL. In one embodiment the concentration of PMPC may be between about 10 μg-1 mg per mL. In one embodiment the concentration of PMPC may be between about 10 μg-500 μg per mL. In one embodiment the concentration of PMPC may be between about 10 μg-250 μg per mL. In one embodiment the concentration of PMPC may be between about 10 μg-100 μg per mL. In one embodiment the concentration of PMPC may be between about 100 μg-1 g per mL. In one embodiment the concentration of PMPC may be between about 100 μg-500 mg per mL. In one embodiment the concentration of PMPC may be between about 100 μg-250 mg per mL. In one embodiment the concentration of PMPC may be between about 100 μg-100 mg per mL. In one embodiment the concentration of PMPC may be between about 100 μg-50 mg per mL. In one embodiment the concentration of PMPC may be between about 100 μg-25 mg per mL. In one embodiment the concentration of PMPC may be between about 100 μg-10 mg per mL. In one embodiment the concentration of PMPC may be between about 100 μg-5 mg per mL. In one embodiment the concentration of PMPC may be between about 100 μg-2.5 mg per mL. In one embodiment the concentration of PMPC may be between about 100 μg-1 mg per mL. In one embodiment the concentration of PMPC may be between about 100 μg-500 μg per mL. In one embodiment the concentration of PMPC may be between about 100 μg-250 μg per mL.

Embodiments of compositions comprising one or more RNA stabilizing substance, such as a stabilizing polymer of which PMPC is a non-limiting example, may have different milligram concentrations. For conciseness, the following list of milligram concentrations with PMPC as a non-limiting example stabilizing polymer, is herein referred to as the stabilizing polymer milligram concentration list.

In one embodiment the concentration of PMPC may be at least 1 ng/mL or more. In one embodiment the concentration of PMPC may be at least 10 ng/mL or more. In one embodiment the concentration of PMPC may be at least 100 ng/mL or more. In one embodiment the concentration of PMPC may be at least 1 μg/mL or more. In one embodiment the concentration of PMPC may be at least 10 μg/mL or more. In one embodiment the concentration of PMPC may be at least 50 μg/mL or more. In one embodiment the concentration of PMPC may be at least 100 μg/mL or more. In one embodiment the concentration of PMPC may be at least 200 μg/mL or more. In one embodiment the concentration of PMPC may be at least 300 μg/mL or more. In one embodiment the concentration of PMPC may be at least 400 μg/mL or more. In one embodiment the concentration of PMPC may be at least 500 μg/mL or more. In one embodiment the concentration of PMPC may be at least 600 μg/mL or more. In one embodiment the concentration of PMPC may be at least 700 μg/mL or more. In one embodiment the concentration of PMPC may be at least 800 μg/mL or more. In one embodiment the concentration of PMPC may be at least 900 μg/mL or more. In one embodiment the concentration of PMPC may be at least 1 mg/mL or more. In one embodiment the concentration of PMPC may be at least 2 mg/mL or more. In one embodiment the concentration of PMPC may be at least 4 mg/mL or more. In one embodiment the concentration of PMPC may be at least 6 mg/mL or more. In one embodiment the concentration of PMPC may be at least 8 mg/mL or more. In one embodiment the concentration of PMPC may be at least 10 mg/mL or more. In one embodiment the concentration of PMPC may be at least 15 mg/mL or more. In one embodiment the concentration of PMPC may be at least 20 mg/mL or more. In one embodiment the concentration of PMPC may be at least 30 mg/mL or more. In one embodiment the concentration of PMPC may be at least 40 mg/mL or more. In one embodiment the concentration of PMPC may be at least 50 mg/mL or more. In one embodiment the concentration of PMPC may be at least 60 mg/mL or more. In one embodiment the concentration of PMPC may be at least 80 mg/mL or more. In one embodiment the concentration of PMPC may be at least 100 mg/mL or more. In one embodiment the concentration of PMPC may be at least 150 mg/mL or more. In one embodiment the concentration of PMPC may be at least 200 mg/mL or more. In one embodiment the concentration of PMPC may be at least 250 mg/mL or more. In one embodiment the concentration of PMPC may be at least 300 mg/mL or more. In one embodiment the concentration of PMPC may be at least 350 mg/mL or more. In one embodiment the concentration of PMPC may be at least 400 mg/mL or more. In one embodiment the concentration of PMPC may be at least 500 mg/mL or more. In one embodiment the concentration of PMPC may be at least 600 mg/mL or more. In one embodiment the concentration of PMPC may be at least 800 mg/mL or more.

Embodiments of compositions comprising one or more RNA stabilizing substance, such as a stabilizing polymer of which PMPC is a non-limiting example, may have different weight percent concentrations. For conciseness, the following list of weight percent concentrations, with PMPC as a non-limiting example stabilizing polymer, is herein referred to as the stabilizing polymer weight percent concentration range list.

In one embodiment the concentration of PMPC may be between about 0.0001%-99.9% of the substances that are not RNA substances (all composition percentages herein are weight percent, unless stated otherwise). In one embodiment the concentration of PMPC may be between about 0.0001%-95% of the substances that are not RNA substances. In one embodiment the concentration of PMPC may be between about 0.0001%-90% of the substances that are not RNA substances. In one embodiment the concentration of PMPC may be between about 0.0001%-80% of the substances that are not RNA substances. In one embodiment the concentration of PMPC may be between about 0.0001%-70% of the substances that are not RNA substances. In one embodiment the concentration of PMPC may be between about 0.0001%-60% of the substances that are not RNA substances. In one embodiment the concentration of PMPC may be between about 0.0001%-50% of the substances that are not RNA substances. In one embodiment the concentration of PMPC may be between about 0.0001%-40% of the substances that are not RNA substances. In one embodiment the concentration of PMPC may be between about 0.0001%-30% of the substances that are not RNA substances. In one embodiment the concentration of PMPC may be between about 0.0001%-25% of the substances that are not RNA substances. In one embodiment the concentration of PMPC may be between about 0.0001%-20% of the substances that are not RNA substances. In one embodiment the concentration of PMPC may be between about 0.0001%-15% of the substances that are not RNA substances. In one embodiment the concentration of PMPC may be between about 0.0001%-10% of the substances that are not RNA substances. In one embodiment the concentration of PMPC may be between about 0.0001%-5% of the substances that are not RNA substances. In one embodiment the concentration of PMPC may be between about 0.0001%-2.5% of the substances that are not RNA substances. In one embodiment the concentration of PMPC may be between about 0.0001%-1% of the substances that are not RNA substances. In one embodiment the concentration of PMPC may be between about 0.0001%-0.5% of the substances that are not RNA substances. In one embodiment the concentration of PMPC may be between about 0.0001%-0.25% of the substances that are not RNA substances. In one embodiment the concentration of PMPC may be between about 0.0001%-0.1% of the substances that are not RNA substances. In one embodiment the concentration of PMPC may be between about 0.0001%-0.05% of the substances that are not RNA substances. In one embodiment the concentration of PMPC may be between about 0.0001%-0.025% of the substances that are not RNA substances. In one embodiment the concentration of PMPC may be between about 0.001%-99.9% (all composition percentages herein are weight percent, unless stated otherwise). In one embodiment the concentration of PMPC may be between about 0.001%-95% of the substances that are not RNA substances. In one embodiment the concentration of PMPC may be between about 0.001%-90% of the substances that are not RNA substances. In one embodiment the concentration of PMPC may be between about 0.001%-80% of the substances that are not RNA substances. In one embodiment the concentration of PMPC may be between about 0.001%-70% of the substances that are not RNA substances. In one embodiment the concentration of PMPC may be between about 0.001%-60% of the substances that are not RNA substances. In one embodiment the concentration of PMPC may be between about 0.001%-50% of the substances that are not RNA substances. In one embodiment the concentration of PMPC may be between about 0.001%-40% of the substances that are not RNA substances. In one embodiment the concentration of PMPC may be between about 0.001%-30% of the substances that are not RNA substances. In one embodiment the concentration of PMPC may be between about 0.001%-25% of the substances that are not RNA substances. In one embodiment the concentration of PMPC may be between about 0.001%-20% of the substances that are not RNA substances. In one embodiment the concentration of PMPC may be between about 0.001%-15% of the substances that are not RNA substances. In one embodiment the concentration of PMPC may be between about 0.001%-10% of the substances that are not RNA substances. In one embodiment the concentration of PMPC may be between about 0.001%-5% of the substances that are not RNA substances. In one embodiment the concentration of PMPC may be between about 0.001%-2.5% of the substances that are not RNA substances. In one embodiment the concentration of PMPC may be between about 0.001%-1% of the substances that are not RNA substances. In one embodiment the concentration of PMPC may be between about 0.001%-0.5% of the substances that are not RNA substances. In one embodiment the concentration of PMPC may be between about 0.001%-0.25% of the substances that are not RNA substances. In one embodiment the concentration of PMPC may be between about 0.001%-0.1% of the substances that are not RNA substances. In one embodiment the concentration of PMPC may be between about 0.001%-0.05% of the substances that are not RNA substances. In one embodiment the concentration of PMPC may be between about 0.001%-0.025% of the substances that are not RNA substances. In one embodiment the concentration of PMPC may be between about 0.01%-99.9% (all composition percentages herein are weight percent, unless stated otherwise). In one embodiment the concentration of PMPC may be between about 0.01%-95% of the substances that are not RNA substances. In one embodiment the concentration of PMPC may be between about 0.01%-90% of the substances that are not RNA substances. In one embodiment the concentration of PMPC may be between about 0.01%-80% of the substances that are not RNA substances. In one embodiment the concentration of PMPC may be between about 0.01%-70% of the substances that are not RNA substances. In one embodiment the concentration of PMPC may be between about 0.01%-60% of the substances that are not RNA substances. In one embodiment the concentration of PMPC may be between about 0.01%-50% of the substances that are not RNA substances. In one embodiment the concentration of PMPC may be between about 0.01%-40% of the substances that are not RNA substances. In one embodiment the concentration of PMPC may be between about 0.01%-30% of the substances that are not RNA substances. In one embodiment the concentration of PMPC may be between about 0.01%-25% of the substances that are not RNA substances. In one embodiment the concentration of PMPC may be between about 0.01%-20% of the substances that are not RNA substances. In one embodiment the concentration of PMPC may be between about 0.01%-15% of the substances that are not RNA substances. In one embodiment the concentration of PMPC may be between about 0.01%-10% of the substances that are not RNA substances. In one embodiment the concentration of PMPC may be between about 0.01%-5% of the substances that are not RNA substances. In one embodiment the concentration of PMPC may be between about 0.01%-2.5% of the substances that are not RNA substances. In one embodiment the concentration of PMPC may be between about 0.01%-1% of the substances that are not RNA substances. In one embodiment the concentration of PMPC may be between about 0.01%-0.5% of the substances that are not RNA substances. In one embodiment the concentration of PMPC may be between about 0.01%-0.25% of the substances that are not RNA substances. In one embodiment the concentration of PMPC may be between about 0.01%-0.1% of the substances that are not RNA substances. In one embodiment the concentration of PMPC may be between about 0.01%-0.05% of the substances that are not RNA substances. In one embodiment the concentration of PMPC may be between about 0.01%-0.025% of the substances that are not RNA substances. In one embodiment the concentration of PMPC may be between about 0.1%-99.9% (all composition percentages herein are weight percent, unless stated otherwise). In one embodiment the concentration of PMPC may be between about 0.1%-95% of the substances that are not RNA substances. In one embodiment the concentration of PMPC may be between about 0.1%-90% of the substances that are not RNA substances. In one embodiment the concentration of PMPC may be between about 0.1%-80% of the substances that are not RNA substances. In one embodiment the concentration of PMPC may be between about 0.1%-70% of the substances that are not RNA substances. In one embodiment the concentration of PMPC may be between about 0.1%-60% of the substances that are not RNA substances. In one embodiment the concentration of PMPC may be between about 0.1%-50% of the substances that are not RNA substances. In one embodiment the concentration of PMPC may be between about 0.1%-40% of the substances that are not RNA substances. In one embodiment the concentration of PMPC may be between about 0.1%-30% of the substances that are not RNA substances. In one embodiment the concentration of PMPC may be between about 0.1%-25% of the substances that are not RNA substances. In one embodiment the concentration of PMPC may be between about 0.1%-20% of the substances that are not RNA substances. In one embodiment the concentration of PMPC may be between about 0.1%-15% of the substances that are not RNA substances. In one embodiment the concentration of PMPC may be between about 0.1%-10% of the substances that are not RNA substances. In one embodiment the concentration of PMPC may be between about 0.1%-5% of the substances that are not RNA substances. In one embodiment the concentration of PMPC may be between about 0.1%-2.5% of the substances that are not RNA substances. In one embodiment the concentration of PMPC may be between about 0.1%-1% of the substances that are not RNA substances. In one embodiment the concentration of PMPC may be between about 0.1%-0.5% of the substances that are not RNA substances. In one embodiment the concentration of PMPC may be between about 0.1%-0.25% of the substances that are not RNA substances. In one embodiment the concentration of PMPC may be between about 1%-99.9% (all composition percentages herein are weight percent, unless stated otherwise). In one embodiment the concentration of PMPC may be between about 1%-95% of the substances that are not RNA substances. In one embodiment the concentration of PMPC may be between about 1%-90% of the substances that are not RNA substances. In one embodiment the concentration of PMPC may be between about 1%-80% of the substances that are not RNA substances. In one embodiment the concentration of PMPC may be between about 1%-70% of the substances that are not RNA substances. In one embodiment the concentration of PMPC may be between about 1%-60% of the substances that are not RNA substances. In one embodiment the concentration of PMPC may be between about 1%-50% of the substances that are not RNA substances. In one embodiment the concentration of PMPC may be between about 1%-40% of the substances that are not RNA substances. In one embodiment the concentration of PMPC may be between about 1%-30% of the substances that are not RNA substances. In one embodiment the concentration of PMPC may be between about 1%-25% of the substances that are not RNA substances. In one embodiment the concentration of PMPC may be between about 1%-20% of the substances that are not RNA substances. In one embodiment the concentration of PMPC may be between about 1%-15% of the substances that are not RNA substances. In one embodiment the concentration of PMPC may be between about 1%-10% of the substances that are not RNA substances. In one embodiment the concentration of PMPC may be between about 1%-5% of the substances that are not RNA substances. In one embodiment the concentration of PMPC may be between about 1%-2.5% of the substances that are not RNA substances. In one embodiment the concentration of PMPC may be between about 2%-99.9% (all composition percentages herein are weight percent, unless stated otherwise). In one embodiment the concentration of PMPC may be between about 2%-95% of the substances that are not RNA substances. In one embodiment the concentration of PMPC may be between about 2%-90% of the substances that are not RNA substances. In one embodiment the concentration of PMPC may be between about 2%-80% of the substances that are not RNA substances. In one embodiment the concentration of PMPC may be between about 2%-70% of the substances that are not RNA substances. In one embodiment the concentration of PMPC may be between about 2%-60% of the substances that are not RNA substances. In one embodiment the concentration of PMPC may be between about 2%-50% of the substances that are not RNA substances. In one embodiment the concentration of PMPC may be between about 2%-40% of the substances that are not RNA substances. In one embodiment the concentration of PMPC may be between about 2%-30% of the substances that are not RNA substances. In one embodiment the concentration of PMPC may be between about 2%-25% of the substances that are not RNA substances. In one embodiment the concentration of PMPC may be between about 2%-20% of the substances that are not RNA substances. In one embodiment the concentration of PMPC may be between about 2%-15% of the substances that are not RNA substances. In one embodiment the concentration of PMPC may be between about 2%-10% of the substances that are not RNA substances. In one embodiment the concentration of PMPC may be between about 2%-5% of the substances that are not RNA substances. In one embodiment the concentration of PMPC may be between about 5%-99.9% (all composition percentages herein are weight percent, unless stated otherwise). In one embodiment the concentration of PMPC may be between about 5%-95% of the substances that are not RNA substances. In one embodiment the concentration of PMPC may be between about 5%-90% of the substances that are not RNA substances. In one embodiment the concentration of PMPC may be between about 5%-80% of the substances that are not RNA substances. In one embodiment the concentration of PMPC may be between about 5%-70% of the substances that are not RNA substances. In one embodiment the concentration of PMPC may be between about 5%-60% of the substances that are not RNA substances. In one embodiment the concentration of PMPC may be between about 5%-50% of the substances that are not RNA substances. In one embodiment the concentration of PMPC may be between about 5%-40% of the substances that are not RNA substances. In one embodiment the concentration of PMPC may be between about 5%-30% of the substances that are not RNA substances. In one embodiment the concentration of PMPC may be between about 5%-25% of the substances that are not RNA substances. In one embodiment the concentration of PMPC may be between about 5%-20% of the substances that are not RNA substances. In one embodiment the concentration of PMPC may be between about 5%-15% of the substances that are not RNA substances. In one embodiment the concentration of PMPC may be between about 5%-10% of the substances that are not RNA substances. In one embodiment the concentration of PMPC may be between about 10%-99.9% (all composition percentages herein are weight percent, unless stated otherwise). In one embodiment the concentration of PMPC may be between about 10%-95% of the substances that are not RNA substances. In one embodiment the concentration of PMPC may be between about 10%-90% of the substances that are not RNA substances. In one embodiment the concentration of PMPC may be between about 10%-80% of the substances that are not RNA substances. In one embodiment the concentration of PMPC may be between about 10%-70% of the substances that are not RNA substances. In one embodiment the concentration of PMPC may be between about 10%-60% of the substances that are not RNA substances. In one embodiment the concentration of PMPC may be between about 10%-50% of the substances that are not RNA substances. In one embodiment the concentration of PMPC may be between about 10%-40% of the substances that are not RNA substances. In one embodiment the concentration of PMPC may be between about 10%-30% of the substances that are not RNA substances. In one embodiment the concentration of PMPC may be between about 10%-25% of the substances that are not RNA substances. In one embodiment the concentration of PMPC may be between about 10%-20% of the substances that are not RNA substances. In one embodiment the concentration of PMPC may be between about 20%-99.9% (all composition percentages herein are weight percent, unless stated otherwise). In one embodiment the concentration of PMPC may be between about 20%-95% of the substances that are not RNA substances. In one embodiment the concentration of PMPC may be between about 20%-90% of the substances that are not RNA substances. In one embodiment the concentration of PMPC may be between about 20%-80% of the substances that are not RNA substances. In one embodiment the concentration of PMPC may be between about 20%-70% of the substances that are not RNA substances. In one embodiment the concentration of PMPC may be between about 20%-60% of the substances that are not RNA substances. In one embodiment the concentration of PMPC may be between about 20%-50% of the substances that are not RNA substances.

Embodiments of compositions comprising one or more RNA stabilizing substance, such as a stabilizing polymer of which PMPC is a non-limiting example, may have different weight percent concentrations. For conciseness, the following list of weight percent concentrations, with PMPC as a non-limiting example stabilizing polymer, is herein referred to as the stabilizing polymer weight percent concentration list.

In one embodiment the concentration of PMPC may be at least about 0.0001% or more of the substances that are not RNA substances (all composition percentages herein are weight percent, unless stated otherwise). In one embodiment the concentration of PMPC may be at least about 0.001% or more of the substances that are not RNA substances. In one embodiment the concentration of PMPC may be at least about 0.01% or more of the substances that are not RNA substances. In one embodiment the concentration of PMPC may be at least about 0.025% or more of the substances that are not RNA substances. In one embodiment the concentration of PMPC may be at least about 0.05% or more of the substances that are not RNA substances. In one embodiment the concentration of PMPC may be at least about 0.1% or more of the substances that are not RNA substances. In one embodiment the concentration of PMPC may be at least about 0.25% or more of the substances that are not RNA substances. In one embodiment the concentration of PMPC may be at least about 0.5% or more of the substances that are not RNA substances. In one embodiment the concentration of PMPC may be at least about 1% or more of the substances that are not RNA substances. In one embodiment the concentration of PMPC may be at least about 2% or more of the substances that are not RNA substances. In one embodiment the concentration of PMPC may be at least about 5% or more of the substances that are not RNA substances. In one embodiment the concentration of PMPC may be at least about 10% or more of the substances that are not RNA substances. In one embodiment the concentration of PMPC may be at least about 15% or more of the substances that are not RNA substances. In one embodiment the concentration of PMPC may be at least about 20% or more of the substances that are not RNA substances. In one embodiment the concentration of PMPC may be at least about 25% or more of the substances that are not RNA substances. In one embodiment the concentration of PMPC may be at least about 30% or more of the substances that are not RNA substances. In one embodiment the concentration of PMPC may be at least about 40% or more of the substances that are not RNA substances. In one embodiment the concentration of PMPC may be at least about 50% or more of the substances that are not RNA substances. In one embodiment the concentration of PMPC may be at least about 60% or more of the substances that are not RNA substances. In one embodiment the concentration of PMPC may be at least about 70% or more of the substances that are not RNA substances. In one embodiment the concentration of PMPC may be at least about 80% or more of the substances that are not RNA substances. In one embodiment the concentration of PMPC may be at least about 90% or more of the substances that are not RNA substances. In one embodiment the concentration of PMPC may be at least about 95% or more of the substances that are not RNA substances. In one embodiment the concentration of PMPC may be at least about 99% or even substantially all of the substances that are not RNA substances.

Embodiments of compositions comprising one or more RNA stabilizing substance, such as a stabilizing polymer of which PMPC is a non-limiting example, may have different N/P ratios of the substance to RNA. For conciseness, the following list of N/P ratios, with PMPC as a non-limiting example stabilizing polymer, is herein referred to as the stabilizing polymer N/P ratio list.

In one embodiment the PMPC to RNA N/P ratio may be greater than about 100,000:1. In one embodiment the PMPC to RNA N/P ratio may be greater than about 10,000:1. In one embodiment the PMPC to RNA N/P ratio may be greater than about 1,000:1. In one embodiment the PMPC to RNA N/P ratio may be greater than about 500:1. In one embodiment the PMPC to RNA N/P ratio may be greater than about 200:1. In one embodiment the PMPC to RNA N/P ratio may be greater than about 100:1. In another embodiment the PMPC to RNA N/P ratio may be greater than about 90:1. In another embodiment the PMPC to RNA N/P ratio may be greater than about 80:1. In another embodiment the PMPC to RNA N/P ratio may be greater than about 70:1. In another embodiment the PMPC to RNA N/P ratio may be greater than about 60:1. In another embodiment the PMPC to RNA N/P ratio may be greater than about 50:1. In another embodiment the PMPC to RNA N/P ratio may be greater than about 40:1. In another embodiment the PMPC to RNA N/P ratio may be greater than about 30:1. In another embodiment the PMPC to RNA N/P ratio may be greater than about 25:1. In another embodiment the PMPC to RNA N/P ratio may be greater than about 20:1. In another embodiment the PMPC to RNA N/P ratio may be greater than about 15:1. In another embodiment the PMPC to RNA N/P ratio may be greater than about 12:1. In another embodiment the PMPC to RNA N/P ratio may be greater than about 10:1. In another embodiment the PMPC to RNA N/P ratio may be greater than about 9:1. In another embodiment the PMPC to RNA N/P ratio may be greater than about 8:1. In another embodiment the PMPC to RNA N/P ratio may be greater than about 7:1 In another embodiment the PMPC to RNA N/P ratio may be greater than about 6:1 In another embodiment the PMPC to RNA N/P ratio may be greater than about 5:1 In another embodiment the PMPC to RNA N/P ratio may be greater than about 4:1 In another embodiment the PMPC to RNA N/P ratio may be greater than about 3:1 In another embodiment the PMPC to RNA N/P ratio may be greater than about 2:1. In another embodiment the PMPC to RNA N/P ratio may be greater than about 1:1. In another embodiment the PMPC to RNA N/P ratio may be greater than about 1:2. In another embodiment the PMPC to RNA N/P ratio may be greater than about 1:3. In another embodiment the PMPC to RNA N/P ratio may be greater than about 1:4. In another embodiment the PMPC to RNA N/P ratio may be greater than about 1:5. In another embodiment the PMPC to RNA N/P ratio may be greater than about 1:6. In another embodiment the PMPC to RNA N/P ratio may be greater than about 1:7. In another embodiment the PMPC to RNA N/P ratio may be greater than about 1:8 In another embodiment the PMPC to RNA N/P ratio may be greater than about 1:9 In another embodiment the PMPC to RNA N/P ratio may be greater than about 1:10 In another embodiment the PMPC to RNA N/P ratio may be greater than about 1:12 In another embodiment the PMPC to RNA N/P ratio may be greater than about 1:15 In another embodiment the PMPC to RNA N/P ratio may be greater than about 1:20 In another embodiment the PMPC to RNA N/P ratio may be greater than about 1:25. In another embodiment the PMPC to RNA N/P ratio may be greater than about 1:30 In another embodiment the PMPC to RNA N/P ratio may be greater than about 1:40 In another embodiment the PMPC to RNA N/P ratio may be greater than about 1:50 In another embodiment the PMPC to RNA N/P ratio may be greater than about 1:60 In another embodiment the PMPC to RNA N/P ratio may be greater than about 1:70. In another embodiment the PMPC to RNA N/P ratio may be greater than about 1:80. In another embodiment the PMPC to RNA N/P ratio may be greater than about 1:90. In another embodiment the PMPC to RNA N/P ratio may be greater than about 1:100. In another embodiment the PMPC to RNA N/P ratio may be greater than about 1:200. In another embodiment the PMPC to RNA N/P ratio may be greater than about 1:500. In another embodiment the PMPC to RNA N/P ratio may be greater than about 1:1,000.

Embodiments of compositions comprising one or more RNA stabilizing substance, such as a stabilizing polymer of which PMPC is a non-limiting example, may have different N/P ratios of the substance to RNA. For conciseness, the following list of N/P ratios, with PMPC as a non-limiting example stabilizing polymer, is herein referred to as the stabilizing polymer N/P ratio range list.

In another embodiment the PMPC to RNA N/P ratio may be between about 1-1,000,000:1. In another embodiment the PMPC to RNA N/P ratio may be between about 1-100,000:1. In another embodiment the PMPC to RNA N/P ratio may be between about 1-10,000:1. In another embodiment the PMPC to RNA N/P ratio may be between about 1-1,000:1. In another embodiment the PMPC to RNA N/P ratio may be between about 1-500:1. In another embodiment the PMPC to RNA N/P ratio may be between about 1-200:1. In another embodiment the PMPC to RNA N/P ratio may be between about 1-100:1. In another embodiment the PMPC to RNA N/P ratio may be between about 1-50:1. In another embodiment the PMPC to RNA N/P ratio may be between about 1-30:1. In another embodiment the PMPC to RNA N/P ratio may be between about 1-25:1. In another embodiment the PMPC to RNA N/P ratio may be between about 1-20:1. In another embodiment the PMPC to RNA N/P ratio may be between about 1-15:1. In another embodiment the PMPC to RNA N/P ratio may be between about 1-10:1. In another embodiment the PMPC to RNA N/P ratio may be between about 1-5:1. In another embodiment the PMPC to RNA N/P ratio may be between about 1-4:1. In another embodiment the PMPC to RNA N/P ratio may be between about 1-2:1.

In another embodiment the PMPC to RNA N/P ratio may be between about 1:1-2. In another embodiment the PMPC to RNA N/P ratio may be between about 1:1-4. In another embodiment the PMPC to RNA N/P ratio may be between about 1:1-6. In another embodiment the PMPC to RNA N/P ratio may be between about 1:1-10. In another embodiment the PMPC to RNA N/P ratio may be between about 1:1-20. In another embodiment the PMPC to RNA N/P ratio may be between about 1:1-30. In another embodiment the PMPC to RNA N/P ratio may be between about 1:1-50. In another embodiment the PMPC to RNA N/P ratio may be between about 1:1-100. In another embodiment the PMPC to RNA N/P ratio may be between about 1:1-200. In another embodiment the PMPC to RNA N/P ratio may be between about 1:1-500. In another embodiment the PMPC to RNA N/P ratio may be between about 1:1-1,000.

As used herein, C/P ratio is the ratio of stabilizing polymer cations or cationic moieties to RNA phosphorus atoms. The C/P ratio is similar to the known art of N/P ratio but can also be used to compare cationic sulfonium moieties and cationic phosphonium moieties.

Embodiments of compositions comprising one or more RNA stabilizing substance, such as a stabilizing polymer of which PMPC is a non-limiting example, may have different C/P ratios of the substance to RNA. For conciseness, the following list of C/P ratios, with PMPC as a non-limiting example stabilizing polymer, is herein referred to as the stabilizing polymer C/P ratio list. In one embodiment the PMPC to RNA C/P ratio may be greater than about 100,000:1. In one embodiment the PMPC to RNA C/P ratio may be greater than about 10,000:1. In one embodiment the PMPC to RNA C/P ratio may be greater than about 1,000:1 In one embodiment the PMPC to RNA C/P ratio may be greater than about 500:1. In one embodiment the PMPC to RNA C/P ratio may be greater than about 200:1. In one embodiment the PMPC to RNA C/P ratio may be greater than about 100:1. In another embodiment the PMPC to RNA C/P ratio may be greater than about 90:1. In another embodiment the PMPC to RNA C/P ratio may be greater than about 80:1. In another embodiment the PMPC to RNA C/P ratio may be greater than about 70:1. In another embodiment the PMPC to RNA C/P ratio may be greater than about 60:1. In another embodiment the PMPC to RNA C/P ratio may be greater than about 50:1. In another embodiment the PMPC to RNA C/P ratio may be greater than about 40:1. In another embodiment the PMPC to RNA C/P ratio may be greater than about 30:1. In another embodiment the PMPC to RNA C/P ratio may be greater than about 25:1. In another embodiment the PMPC to RNA C/P ratio may be greater than about 20:1. In another embodiment the PMPC to RNA C/P ratio may be greater than about 15:1. In another embodiment the PMPC to RNA C/P ratio may be greater than about 12:1. In another embodiment the PMPC to RNA C/P ratio may be greater than about 10:1. In another embodiment the PMPC to RNA C/P ratio may be greater than about 9:1. In another embodiment the PMPC to RNA C/P ratio may be greater than about 8:1. In another embodiment the PMPC to RNA C/P ratio may be greater than about 7:1 In another embodiment the PMPC to RNA C/P ratio may be greater than about 6:1 In another embodiment the PMPC to RNA C/P ratio may be greater than about 5:1 In another embodiment the PMPC to RNA C/P ratio may be greater than about 4:1 In another embodiment the PMPC to RNA C/P ratio may be greater than about 3:1 In another embodiment the PMPC to RNA C/P ratio may be greater than about 2:1. In another embodiment the PMPC to RNA C/P ratio may be greater than about 1:1. In another embodiment the PMPC to RNA C/P ratio may be greater than about 1:2. In another embodiment the PMPC to RNA C/P ratio may be greater than about 1:3. In another embodiment the PMPC to RNA C/P ratio may be greater than about 1:4. In another embodiment the PMPC to RNA C/P ratio may be greater than about 1:5. In another embodiment the PMPC to RNA C/P ratio may be greater than about 1:6. In another embodiment the PMPC to RNA C/P ratio may be greater than about 1:7. In another embodiment the PMPC to RNA C/P ratio may be greater than about 1:8 In another embodiment the PMPC to RNA C/P ratio may be greater than about 1:9 In another embodiment the PMPC to RNA C/P ratio may be greater than about 1:10 In another embodiment the PMPC to RNA C/P ratio may be greater than about 1:12 In another embodiment the PMPC to RNA C/P ratio may be greater than about 1:15 In another embodiment the PMPC to RNA C/P ratio may be greater than about 1:20 In another embodiment the PMPC to RNA C/P ratio may be greater than about 1:25. In another embodiment the PMPC to RNA C/P ratio may be greater than about 1:30 In another embodiment the PMPC to RNA C/P ratio may be greater than about 1:40 In another embodiment the PMPC to RNA C/P ratio may be greater than about 1:50 In another embodiment the PMPC to RNA C/P ratio may be greater than about 1:60 In another embodiment the PMPC to RNA C/P ratio may be greater than about 1:70. In another embodiment the PMPC to RNA C/P ratio may be greater than about 1:80. In another embodiment the PMPC to RNA C/P ratio may be greater than about 1:90. In another embodiment the PMPC to RNA C/P ratio may be greater than about 1:100. In another embodiment the PMPC to RNA C/P ratio may be greater than about 1:200. In another embodiment the PMPC to RNA C/P ratio may be greater than about 1:500. In another embodiment the PMPC to RNA C/P ratio may be greater than about 1:1,000.

Embodiments of compositions comprising one or more RNA stabilizing substance, such as a stabilizing polymer of which PMPC is a non-limiting example, may have different C/P ratios of the substance to RNA. For conciseness, the following list of C/P ratios, with PMPC as a non-limiting example stabilizing polymer, is herein referred to as the stabilizing polymer C/P ratio range list.

In another embodiment the PMPC to RNA C/P ratio may be between about 1-1,000,000:1. In another embodiment the PMPC to RNA C/P ratio may be between about 1-100,000:1. In another embodiment the PMPC to RNA C/P ratio may be between about 1-10,000:1. In another embodiment the PMPC to RNA C/P ratio may be between about 1-1,000:1. In another embodiment the PMPC to RNA C/P ratio may be between about 1-500:1. In another embodiment the PMPC to RNA C/P ratio may be between about 1-200:1. In another embodiment the PMPC to RNA C/P ratio may be between about 1-100:1. In another embodiment the PMPC to RNA C/P ratio may be between about 1-50:1. In another embodiment the PMPC to RNA C/P ratio may be between about 1-30:1. In another embodiment the PMPC to RNA C/P ratio may be between about 1-25:1. In another embodiment the PMPC to RNA C/P ratio may be between about 1-20:1. In another embodiment the PMPC to RNA C/P ratio may be between about 1-15:1. In another embodiment the PMPC to RNA C/P ratio may be between about 1-10:1. In another embodiment the PMPC to RNA C/P ratio may be between about 1-5:1. In another embodiment the PMPC to RNA C/P ratio may be between about 1-4:1. In another embodiment the PMPC to RNA C/P ratio may be between about 1-2:1.

In another embodiment the PMPC to RNA C/P ratio may be between about 1:1-2. In another embodiment the PMPC to RNA C/P ratio may be between about 1:1-4. In another embodiment the PMPC to RNA C/P ratio may be between about 1:1-6. In another embodiment the PMPC to RNA C/P ratio may be between about 1:1-10. In another embodiment the PMPC to RNA C/P ratio may be between about 1:1-20. In another embodiment the PMPC to RNA C/P ratio may be between about 1:1-30. In another embodiment the PMPC to RNA C/P ratio may be between about 1:1-50. In another embodiment the PMPC to RNA C/P ratio may be between about 1:1-100. In another embodiment the PMPC to RNA C/P ratio may be between about 1:1-200. In another embodiment the PMPC to RNA C/P ratio may be between about 1:1-500. In another embodiment the PMPC to RNA C/P ratio may be between about 1:1-1,000.

In one embodiment a stabilizing polymer may comprise one or more quaternary ammonium. In one embodiment a stabilizing polymer may comprise one or more tertiary amine. In one embodiment a stabilizing polymer may comprise one or more tertiary sulfonium. In one embodiment a stabilizing polymer may comprise one or more quaternary phosphonium.

In one embodiment a stabilizing polymer may comprise one or more quaternary ammonium cation or cationic moiety. In one embodiment a stabilizing polymer may comprise one or more tertiary sulfonium cation or cationic moiety. In one embodiment a stabilizing polymer may comprise one or more quaternary phosphonium cation or cationic moiety.

In one embodiment a stabilizing polymer may comprise one or more aliphatic quaternary ammonium. In one embodiment a stabilizing polymer may comprise one or more aliphatic tertiary amine. In one embodiment a stabilizing polymer may comprise one or more quaternary ammonium nitrogen heterocycle. In one embodiment a stabilizing polymer may comprise one or more quaternary ammonium aromatic nitrogen heterocycle. In one embodiment a stabilizing polymer may comprise one or more five membered quaternary ammonium nitrogen heterocycle. In one embodiment a stabilizing polymer may comprise one or more six membered quaternary ammonium nitrogen heterocycle. In one embodiment a stabilizing polymer may comprise one or more five membered aromatic quaternary ammonium nitrogen heterocycle. In one embodiment a stabilizing polymer may comprise one or more six membered aromatic quaternary ammonium nitrogen heterocycle.

In one embodiment a stabilizing polymer may comprise one or more cation or cationic moiety. In one embodiment a stabilizing polymer may comprise one or more anion or anionic moiety. In one embodiment a stabilizing polymer may comprise one or more zwitterion or zwitterionic moiety. In one embodiment a stabilizing polymer may be zwitterionic.

In one embodiment a stabilizing polymer may comprise one or more betaine. In one embodiment a stabilizing polymer may comprise one or more carboxybetaine. In one embodiment a stabilizing polymer may comprise one or more sulfobetaine, such as a non-detergent sulfobetaine as a non-limiting example.

In one embodiment a stabilizing polymer may comprise one or more quaternary ammonium cation that may be at least part of one or more nitrogen heterocycle or aromatic nitrogen heterocycle, such as a pyridinium, pyrrolidinium, imidazolium, piperidinium, indolium, pyrimidinium, or purinium group as non-limiting examples.

In one embodiment a stabilizing polymer may comprise one or more nitrogen heterocycle or aromatic nitrogen heterocycle, such as a pyridinium, pyrrolidinium, imidazolium, piperidinium, indolium, pyrimidinium, or purinium group as non-limiting examples.

In one embodiment a stabilizing polymer may comprise one or more carboxylate group. In one embodiment a stabilizing polymer may comprise one or more carboxylate ester. In one embodiment a stabilizing polymer may comprise one or more sulfonate group. In one embodiment a stabilizing polymer may comprise one or more organophosphate group. In one embodiment a stabilizing polymer may comprise one or more organosulfate group.

In one embodiment a stabilizing polymer may comprise one or more carboxylate anion or anionic moiety. In one embodiment a stabilizing polymer may comprise one or more sulfonate anion or anionic moiety. In one embodiment a stabilizing polymer may comprise one or more organophosphate anion or anionic moiety. In one embodiment a stabilizing polymer may comprise one or more organosulfate anion or anionic moiety.

### Aprotic Stabilizing Polymer

In one embodiment a stabilizing polymer may comprise one or more aprotic substance. In one embodiment a stabilizing polymer may comprise one or more polar substance. In one embodiment a stabilizing polymer may comprise one or more polar aprotic substance. In one embodiment a stabilizing polymer may be aprotic or at least partially aprotic at about physiologic pH.

A non-limiting example of an aprotic or at least partially aprotic stabilizing polymer, comprised of one or more aprotic substance, that may be used is poly(vinylpyrrolidone) (PVP) (also known as PVP or povidone), such as poly(vinylpyrrolidone) (˜10 kDa) as a non-limiting example, wherein PVP may be substituted for or used in combination with one or more stabilizing polymer as described herein. One embodiment of the present invention may include combinations of substances comprising PVP and another embodiment may also comprise at least one RNA substance. Other embodiments may include combinations of substances comprising PVP wherein the concentration of PVP may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising PVP wherein the ratio of PVP to one or more RNA substance may include one or more ratio in the stabilizing polymer N/P ratio list, or in the stabilizing polymer N/P ratio range list, or in the stabilizing polymer C/P ratio list, or in the stabilizing polymer C/P ratio range list. Other embodiments may include combinations of substances comprising PVP wherein the concentration of PVP may be one or more concentration in the stabilizing substance weight percent concentration list or in the stabilizing substance molar concentration range list or in the stabilizing substance molar concentration list.

### Anionic Stabilizing Polymer

In one embodiment a stabilizing polymer may comprise one or more COPS stabilizing moiety. In one embodiment a stabilizing polymer may comprise one or more anionic stabilizing monomer.

In one embodiment a stabilizing polymer may comprise one or more anionic moiety. In one embodiment a stabilizing polymer may comprise one or more anionic moiety at about physiologic pH.

In one embodiment a stabilizing polymer may be anionic or polyanionic. In one embodiment a stabilizing polymer may be anionic or polyanionic at about physiologic pH. In one embodiment a stabilizing polymer may be at least partially anionic. In one embodiment a stabilizing polymer may be at least partially anionic at about physiologic pH.

In one embodiment a stabilizing polymer may comprise one or more of the following moieties: carboxylic acid, carboxylate, carboxylate ester, organophosphate, phosphonic acid, phosphonate, sulfonic acid, sulfonate, or organosulfate.

A non-limiting example of an anionic or polyanionic RNA stabilizing polymer, comprised of one or more carboxylic acid or carboxylate, that may be used is poly(acrylic acid) (also known as PAA), such as poly(acrylic acid) sodium salt (˜8 kDa) as a non-limiting example, wherein PAA may be substituted for or used in combination with one or more stabilizing polymer as described herein. One embodiment of the present invention may include combinations of substances comprising PAA and another embodiment may also comprise at least one RNA substance. Other embodiments may include combinations of substances comprising PAA wherein the concentration of PAA may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising PAA wherein the concentration of PAA may be one or more concentration in the stabilizing substance weight percent concentration list or in the stabilizing substance molar concentration range list or in the stabilizing substance molar concentration list.

### Cationic Stabilizing Polymer

In one embodiment a stabilizing polymer may comprise one or more NPS stabilizing moiety. In one embodiment a stabilizing polymer may comprise one or more cationic stabilizing monomer.

In one embodiment a stabilizing polymer may comprise one or more cationic moiety. In one embodiment a stabilizing polymer may comprise one or more cationic moiety at about physiologic pH.

In one embodiment a stabilizing polymer may be cationic or polycationic. In one embodiment a stabilizing polymer may be cationic or polycationic at about physiologic pH. In one embodiment a stabilizing polymer may be at least partially cationic. In one embodiment a stabilizing polymer may be at least partially cationic at about physiologic pH.

In one embodiment a stabilizing polymer may comprise one or more quaternary ammonium. In one embodiment a stabilizing polymer may comprise one or more tertiary sulfonium. In one embodiment a stabilizing polymer may comprise one or more quaternary phosphonium. In one embodiment a stabilizing polymer may comprise one or more tertiary amine.

In one embodiment a stabilizing polymer may comprise one or more ring structure. In one embodiment a stabilizing polymer may comprise one or more heterocyclic ring structure. In one embodiment a stabilizing polymer may comprise one or more five membered ring structure. In one embodiment a stabilizing polymer may comprise one or more six membered ring structure. In one embodiment a stabilizing polymer may comprise one or more heterocyclic six membered ring structure. In one embodiment a stabilizing polymer may comprise one or more heterocyclic five membered ring structure. In one embodiment a stabilizing polymer may comprise one or more nitrogen heterocycles.

In one embodiment a stabilizing polymer may comprise one or more aromatic ring structure. In one embodiment a stabilizing polymer may comprise one or more aromatic heterocyclic ring structure. In one embodiment a stabilizing polymer may comprise one or more aromatic five membered ring structure. In one embodiment a stabilizing polymer may comprise one or more aromatic six membered ring structure. In one embodiment a stabilizing polymer may comprise one or more aromatic heterocyclic six membered ring structure. In one embodiment a stabilizing polymer may comprise one or more aromatic heterocyclic five membered ring structure. In one embodiment a stabilizing polymer may comprise one or more aromatic nitrogen heterocycles.

In one embodiment a stabilizing polymer may comprise one or more pyridine or pyridinium group. In one embodiment a stabilizing polymer may comprise one or more pyrrolidine or pyrrolidinium group. In one embodiment a stabilizing polymer may comprise one or more imidazole or imidazolium group. In one embodiment a stabilizing polymer may comprise one or more piperidine or piperidinium group. In one embodiment a stabilizing polymer may comprise one or more pyrimidine or pyrimidinium group.

In one embodiment a stabilizing polymer may comprise one or more quaternary ammonium cation or tertiary amine that may be at least part of one or more nitrogen heterocycle, such as a pyridine, pyridinium, pyrrolidine, pyrrolidinium, pyrrole, imidazole, imidazolium, pyrazole, pyrimidine, pyrimidinium, piperidine, piperidinium as non-limiting examples.

In one embodiment a stabilizing polymer may not comprise a nucleic acid or nucleic base.

A non-limiting example of a cationic or polycationic stabilizing polymer, comprised of one or more quaternary ammonium, that may be used is poly(diallyldimethylammonium) (PDADMAC) (also known as, PDADMAC or poly(diallyldimethylammonium chloride)), such as poly(diallyldimethylammonium chloride) (˜8.5 kDa) as a non-limiting example, wherein PDADMAC may be substituted for or used in combination with one or more stabilizing polymer as described herein. One embodiment of the present invention may include combinations of substances comprising PDADMAC and another embodiment may also comprise at least one RNA substance. Other embodiments may include combinations of substances comprising PDADMAC wherein the concentration of PDADMAC may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising PDADMAC wherein the ratio of PDADMAC to one or more RNA substance may include one or more ratio in the stabilizing polymer N/P ratio list, or in the stabilizing polymer N/P ratio range list, or in the stabilizing polymer C/P ratio list, or in the stabilizing polymer C/P ratio range list. Other embodiments may include combinations of substances comprising PDADMAC wherein the concentration of PDADMAC may be one or more concentration in the stabilizing substance weight percent concentration list or in the stabilizing substance molar concentration range list or in the stabilizing substance molar concentration list

A non-limiting example of a cationic or polycationic stabilizing polymer, comprised of one or more quaternary ammonium, that may be used is poly(2-(trimethylamino)ethyl methacrylate) (PTMAEMA) (also known as, PTMAEMA, poly(2-dimethylamino)ethyl methacrylate) methyl chloride quaternary salt, poly(2-methacryloxyethyltrimethylammonium chloride), or MADQUAT), such as poly(2-(trimethylamino)ethyl methacrylate) chloride (˜8.5 kDa) as a non-limiting example, wherein PTMAEMA may be substituted for or used in combination with one or more stabilizing polymer as described herein. One embodiment of the present invention may include combinations of substances comprising PTMAEMA and another embodiment may also comprise at least one RNA substance. Other embodiments may include combinations of substances comprising PTMAEMA wherein the concentration of PTMAEMA may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising PTMAEMA wherein the ratio of PTMAEMA to one or more RNA substance may include one or more ratio in the stabilizing polymer N/P ratio list, or in the stabilizing polymer N/P ratio range list, or in the stabilizing polymer C/P ratio list, or in the stabilizing polymer C/P ratio range list. Other embodiments may include combinations of substances comprising PTMAEMA wherein the concentration of PTMAEMA may be one or more concentration in the stabilizing substance weight percent concentration list or in the stabilizing substance molar concentration range list or in the stabilizing substance molar concentration list.

A non-limiting example of a cationic or polycationic stabilizing polymer, comprised of one or more quaternary ammonium, that may be used is hexadimethrine (also known as Polybrene, hexadimethrine bromide or 1,5-dimethyl-1,5-diazaundecamethylene polymethobromide), such as hexadimethrine bromide (˜5 kDa) as a non-limiting example, wherein hexadimethrine may be substituted for or used in combination with one or more stabilizing polymer as described herein. One embodiment of the present invention may include combinations of substances comprising hexadimethrine and another embodiment may also comprise at least one RNA substance. Other embodiments may include combinations of substances comprising hexadimethrine wherein the concentration of hexadimethrine may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising hexadimethrine wherein the ratio of hexadimethrine to one or more RNA substance may include one or more ratio in the stabilizing polymer N/P ratio list, or in the stabilizing polymer N/P ratio range list, or in the stabilizing polymer C/P ratio list, or in the stabilizing polymer C/P ratio range list. Other embodiments may include combinations of substances comprising hexadimethrine wherein the concentration of hexadimethrine may be one or more concentration in the stabilizing substance weight percent concentration list or in the stabilizing substance molar concentration range list or in the stabilizing substance molar concentration list.

A non-limiting example of a stabilizing polymer comprised of a tertiary amine, comprised of one or more stabilizing monomer or NPS stabilizing moiety, that may be used is poly(2-(diethylamino)ethyl methacrylate) (PDEAEMA), such as poly(2-(diethylamino)ethyl methacrylate) (˜10 kDa) as a non-limiting example, wherein PDEAEMA may be substituted for or used in combination with one or more stabilizing polymer as described herein. One embodiment of the present invention may include combinations of substances comprising PDEAEMA and another embodiment may also comprise at least one RNA substance. Other embodiments may include combinations of substances comprising PDEAEMA wherein the concentration of PDEAEMA may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising PDEAEMA wherein the ratio of PDEAEMA to one or more RNA substance may include one or more ratio in the stabilizing polymer N/P ratio list, or in the stabilizing polymer N/P ratio range list, or in the stabilizing polymer C/P ratio list, or in the stabilizing polymer C/P ratio range list. Other embodiments may include combinations of substances comprising PDEAEMA wherein the concentration of PDEAEMA may be one or more concentration in the stabilizing substance weight percent concentration list or in the stabilizing substance molar concentration range list or in the stabilizing substance molar concentration list.

A non-limiting example of a cationic or polycationic stabilizing polymer, comprised of one or more stabilizing monomer, that may be used is poly((carboxybetaine methacrylate)ethyl ester)) (PCBMA-ethyl ester), such as PCBMA-ethyl ester (˜10 kDa) as a non-limiting example, wherein PCBMA-ethyl ester may be substituted for or used in combination with one or more stabilizing polymer as described herein. One embodiment of the present invention may include combinations of substances comprising PCBMA-ethyl ester and another embodiment may also comprise at least one RNA substance. Other embodiments may include combinations of substances comprising PCBMA-ethyl ester wherein the concentration of PCBMA-ethyl ester may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising PCBMA-ethyl ester wherein the ratio of PCBMA-ethyl ester to one or more RNA substance may include one or more ratio in the stabilizing polymer N/P ratio list, or in the stabilizing polymer N/P ratio range list, or in the stabilizing polymer C/P ratio list, or in the stabilizing polymer C/P ratio range list. Other embodiments may include combinations of substances comprising PCBMA-ethyl ester wherein the concentration of PCBMA-ethyl ester may be one or more concentration in the stabilizing substance weight percent concentration list or in the stabilizing substance molar concentration range list or in the stabilizing substance molar concentration list.

### Zwitterionic Stabilizing Polymer

In one embodiment a stabilizing polymer may comprise one or more stabilizing monomer. In one embodiment a stabilizing polymer may comprise one or more NPS stabilizing moiety and one or more COPS stabilizing moiety.

In one embodiment a stabilizing polymer may comprise one or more NPS stabilizing moiety. In one embodiment a stabilizing polymer may comprise one or more zwitterionic stabilizing monomer.

In one embodiment a stabilizing polymer may comprise one or more zwitterionic moiety. In one embodiment a stabilizing polymer may comprise one or more zwitterionic moiety at about physiologic pH.

In one embodiment a stabilizing polymer may be zwitterionic or polyzwitterionic. In one embodiment a stabilizing polymer may be zwitterionic or polyzwitterionic at about physiologic pH. In one embodiment a stabilizing polymer may be at least partially zwitterionic. In one embodiment a stabilizing polymer may be at least partially zwitterionic at about physiologic pH.

In one embodiment a stabilizing polymer may comprise one or more cationic moiety, such as a quaternary ammonium, tertiary sulfonium, or quaternary phosphonium, as non-limiting examples, and one or more anionic moiety, such as a carboxylic acid, carboxylate, organophosphate, phosphonic acid, phosphonate, sulfonic acid, sulfonate, or organosulfate, as non-limiting examples.

A non-limiting example of a zwitterionic or at least partially zwitterionic stabilizing polymer, comprised of one or more stabilizing monomer, that may be used is poly(2-(N-3-sulfopropyl-N,N-dimethyl ammonium)ethyl methacrylate) (PSBMA) (also known as PSBMA or poly(sulfobetaine methacrylate)), such as PSBMA (˜7.5 kDa) as a non-limiting example, wherein PSBMA may be substituted for or used in combination with one or more stabilizing polymer as described herein. One embodiment of the present invention may include combinations of substances comprising PSBMA and another embodiment may also comprise at least one RNA substance. Other embodiments may include combinations of substances comprising PSBMA wherein the concentration of PSBMA may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising PSBMA wherein the ratio of PSBMA to one or more RNA substance may include one or more ratio in the stabilizing polymer N/P ratio list, or in the stabilizing polymer N/P ratio range list, or in the stabilizing polymer C/P ratio list, or in the stabilizing polymer C/P ratio range list. Other embodiments may include combinations of substances comprising PSBMA wherein the concentration of PSBMA may be one or more concentration in the stabilizing substance weight percent concentration list or in the stabilizing substance molar concentration range list or in the stabilizing substance molar concentration list.

A non-limiting example of a zwitterionic or at least partially zwitterionic stabilizing polymer, comprised of one or more stabilizing monomer, that may be used is poly(carboxybetaine methacrylate) (also known as PCBMA), such as PCBMA (˜8 kDa) as a non-limiting example, wherein PCBMA may be substituted for or used in combination with one or more stabilizing polymer as described herein. One embodiment of the present invention may include combinations of substances comprising PCBMA and another embodiment may also comprise at least one RNA substance. Other embodiments may include combinations of substances comprising PCBMA wherein the concentration of PCBMA may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising PCBMA wherein the ratio of PCBMA to one or more RNA substance may include one or more ratio in the stabilizing polymer N/P ratio list, or in the stabilizing polymer N/P ratio range list, or in the stabilizing polymer C/P ratio list, or in the stabilizing polymer C/P ratio range list. Other embodiments may include combinations of substances comprising PCBMA wherein the concentration of PCBMA may be one or more concentration in the stabilizing substance weight percent concentration list or in the stabilizing substance molar concentration range list or in the stabilizing substance molar concentration list

A non-limiting example of a zwitterionic or at least partially zwitterionic stabilizing polymer, comprised of one or more stabilizing monomer, that may be used is poly(vinyl-pyridinio propanesulfonate) (PVPPS), such as PVPPS (˜8 kDa) as a non-limiting example, wherein PVPPS may be substituted for or used in combination with one or more stabilizing polymer as described herein. One embodiment of the present invention may include combinations of substances comprising PVPPS and another embodiment may also comprise at least one RNA substance. Other embodiments may include combinations of substances comprising PVPPS wherein the concentration of PVPPS may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising PVPPS wherein the ratio of PVPPS to one or more RNA substance may include one or more ratio in the stabilizing polymer N/P ratio list, or in the stabilizing polymer N/P ratio range list, or in the stabilizing polymer C/P ratio list, or in the stabilizing polymer C/P ratio range list. Other embodiments may include combinations of substances comprising PVPPS wherein the concentration of PVPPS may be one or more concentration in the stabilizing substance weight percent concentration list or in the stabilizing substance molar concentration range list or in the stabilizing substance molar concentration list

### Stabilizing Polymer Comprising a Copolymer or Block Copolymer

A non-limiting example of a stabilizing polymer, comprised of one or more copolymer or block copolymer, that may be used is poly(ethylene glycol)-block-poly(sulfobetaine methacrylate) (PEG-PSBMA) (also known as PEG-block-PSBMA), such as PEG-PSBMA (PEG Mₙ 5,000; PSBMA Mₙ 13,000) as a non-limiting example, wherein PEG-PSBMA may be substituted for or used in combination with one or more stabilizing polymer as described herein. One embodiment of the present invention may include combinations of substances comprising PEG-PSBMA and another embodiment may also comprise at least one RNA substance. Other embodiments may include combinations of substances comprising PEG-PSBMA wherein the concentration of PEG-PSBMA may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising PEG-PSBMA wherein the ratio of PEG-PSBMA to one or more RNA substance may include one or more ratio in the stabilizing polymer N/P ratio list, or in the stabilizing polymer N/P ratio range list, or in the stabilizing polymer C/P ratio list, or in the stabilizing polymer C/P ratio range list. Other embodiments may include combinations of substances comprising PEG-PSBMA wherein the concentration of PEG-PSBMA may be one or more concentration in the stabilizing substance weight percent concentration list or in the stabilizing substance molar concentration range list or in the stabilizing substance molar concentration list

A non-limiting example of a stabilizing polymer, comprised of one or more copolymer or block copolymer, that may be used is poly(ethylene glycol)-block-poly(2-methacryloyloxyethyl phosphorylcholine) (PEG-PMPC) (also known as PEG-block-PMPC), such as PEG-PSBMA (PEG Mₙ 5,000; PMPC Mₙ 21,000) as a non-limiting example, wherein PEG-PMPC may be substituted for or used in combination with one or more stabilizing polymer as described herein. One embodiment of the present invention may include combinations of substances comprising PEG-PMPC and another embodiment may also comprise at least one RNA substance. Other embodiments may include combinations of substances comprising PEG-PMPC wherein the concentration of PEG-PMPC may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising PEG-PMPC wherein the ratio of PEG-PMPC to one or more RNA substance may include one or more ratio in the stabilizing polymer N/P ratio list, or in the stabilizing polymer N/P ratio range list, or in the stabilizing polymer C/P ratio list, or in the stabilizing polymer C/P ratio range list. Other embodiments may include combinations of substances comprising PEG-PMPC wherein the concentration of PEG-PMPC may be one or more concentration in the stabilizing substance weight percent concentration list or in the stabilizing substance molar concentration range list or in the stabilizing substance molar concentration list

A non-limiting example of a stabilizing polymer, comprised of one or more copolymer or block copolymer, that may be used is poly[(2-ethyldimethylammonioethyl methacrylate ethyl sulfate)-co-(1-vinylpyrrolidone)] (Polyquat 11) (also known as poly(l-vinylpyrrolidone-co-2-dimethylaminoethyl methacrylate), or polyquaternium 11), wherein Polyquat 11 may be substituted for or used in combination with one or more stabilizing polymer as described herein. One embodiment of the present invention may include combinations of substances comprising Polyquat 11 and another embodiment may also comprise at least one RNA substance. Other embodiments may include combinations of substances comprising Polyquat 11 wherein the concentration of Polyquat 11 may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising Polyquat 11 wherein the ratio of Polyquat 11 to one or more RNA substance may include one or more ratio in the stabilizing polymer N/P ratio list, or in the stabilizing polymer N/P ratio range list, or in the stabilizing polymer C/P ratio list, or in the stabilizing polymer C/P ratio range list. Other embodiments may include combinations of substances comprising Polyquat 11 wherein the concentration of Polyquat 11 may be one or more concentration in the stabilizing substance weight percent concentration list or in the stabilizing substance molar concentration range list or in the stabilizing substance molar concentration list

For conciseness, the following list of stabilizing polymers, is herein referred to as the stabilizing polymer list wherein one or more of the following substances may be substituted for or used in combination with one or more stabilizing polymer as described herein.

In other embodiments, other stabilizing polymers that may be substituted for or used in combination with one or more stabilizing polymer as described herein may comprise one or more of the following, including but not limited to: poly(2-acryloyloxyethyl phosphorylcholine), poly(acryloyloxyethyl phosphorylcholine), quaternary ammonium polymethacrylate, quaternary phosphonium polymethacrylate, tertiary sulfonium methacrylate, quaternary ammonium polyacrylate, quaternary phosphonium polyacrylate, tertiary sulfonium polyacrylate, poly(2-methacryloxyethyldimethylsulfonium), poly(carboxybetaine), poly(sulfobetaine), poly(phosphocholine), poly(phosphorylcholine), poly(dimethylsulfoniopropionate), poly(2-methacryloyloxyethyl phosphorylcholine), poly(carboxybetaine acrylamide), poly(carboxybetaine methacrylate), poly(phosphobetaine methacrylate), poly(sulfobetaine methacrylate), poly(methacryloyloxyethyl phosphorylcholine), poly(vinyl-pyridinio propanesulfonate), poly(carboxybetaine vinylimidazole), poly(sulfobetaine vinylimidazole), poly(sulfobetaine vinylpyridine), carboxybetaine, sulfobetaine, phosphobetaine, phosphocholine, phosphorylcholine, dimethylsulfoniopropionate, poly(lactic acid sulfobetaine), poly(methacryloyloxyethyl phosphorylcholine)-poly(lactic acid), poly(4-vinylbenzyl)tributylphosphonium, triethylphosphonium poly(methacrylate), poly(triethylphosphonium methacrylate), triphenylphosphonium polyethylenimine, poly(triphenylphosphonium polyethylenimine), N-phosphonium chitosan, poly[(2-acryloxy)ethyltributylphosphonium], poly(2-methacryloxytriethylphosphonium), poly(2-methacryloxytributylphosphonium), poly(2-methacryloxytrimethylphosphonium), poly(2-acryloxytriethylphosphonium), poly(2-acryloxytributylphosphonium), poly(2-acryloxytrimethylphosphonium), poly(trimethyl-(4-vinylbenzyl)ammonium), poly(triethyl-(4-vinylbenzyl)ammonium), poly(tributyl-(4-vinylbenzyl)ammonium), poly(trimethyl-(4-vinylbenzyl)phosphonium), poly(triethyl-(4-vinylbenzyl)phosphonium), poly(tributyl-(4-vinylbenzyl)phosphonium), poly(dimethyl-(4-vinylbenzyl)sulfonium), poly(diethyl-(4-vinylbenzyl)sulfonium), and poly(dibutyl-(4-vinylbenzyl)sulfonium), poly(phosphorylcholine), poly(sulfobetaine), poly(carboxybetaine), poly(dimethylsulfonioproprionate), poly(dimethlysulfonium ethylmethacrylate), poly(carboxybetaine acrylamide), poly(carboxybetaine metacrylate), poly(sulfobetaine methacrylate), poly(methacyloyloxyethyl phosphorylcholine), poly(vinyl-pyridinio propanesulfonate), poly(carboxybetaine vinylimidazole), poly(sulfobetaine vinylpyridine), poly[(2-ethyldimethylammonioethyl methacrylate ethyl sulfate)-co-(1-vinylpyrrolidone)], poly(diallyldimethylammonium chloride), poly(2-methacryloxyethyltrimethylammonium chloride), poly[(2-ethyldimethylammonioethyl methacrylate ethyl sulfate)-co-(1-vinylpyrrolidone)], poly(vinylpyrrolidone), poly(acrylic acid), poly(styrenesulfonic acid), poly(methacrylate), hexadimethrine, poly(diallyldimethylammonium), poly(2-(trimethylamino)ethyl methacrylate), poly(2-(N-3- sulfopropyl-N,N-dimethyl ammonium)ethyl methacrylate), poly(carboxybetaine methacrylate), poly(2-methacryloyloxyethyl phosphorylcholine), poly(vinyl-pyridinio propanesulfonate), poly(2-(dimethylamino)ethyl methacrylate), poly(2-(diethylamino)ethyl methacrylate), poly(2-(diisopropylamino)ethyl methacrylate), or polyquaterniums (including polyquaterniums 1-3, or polyquaterniums 5-9, or polyquaterniums 11-23, or polyquaterniums 25-28, or polyquaterniums 30-47, as non-limiting examples), or salts, or derivatives, or combinations thereof.

Embodiments of the present invention may include combinations comprising one or more of the polymers from the stabilizing polymer list substituted for or used in combination with one or more stabilizing polymer or one or more RNA stabilizing substance, such as a hydrotrope containing substance as a non-limiting example, and other embodiments may also comprise at least one RNA substance.

Other embodiments may include combinations of substances comprising one or more stabilizing polymer selected from the stabilizing polymer list wherein the concentration of one or more polymers may be one or more concentration in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. Other embodiments may include combinations of substances comprising one or more stabilizing polymer selected from the stabilizing polymer list wherein the ratio of one or more polymer to one or more RNA substance may include one or more ratio in the stabilizing polymer N/P ratio list, or in the stabilizing polymer N/P ratio range list, or in the stabilizing polymer C/P ratio list, or in the stabilizing polymer C/P ratio range list. Other embodiments may include combinations of substances comprising one or more stabilizing polymer selected from the stabilizing polymer list wherein the concentration of one or more polymers may be one or more concentration in the stabilizing substance weight percent concentration list or in the stabilizing substance molar concentration range list or in the stabilizing substance molar concentration list.

As used herein, a stabilizing complex comprises one or more RNA stabilizing substance, such as a stabilizing polymer as a non-limiting example, wherein at least one or more RNA substance may be at least partially embedded in or at least partially bound to or at least partially complexed with one or more RNA stabilizing substance.

In other embodiments, one or more compositions may comprise a stabilizing complex, wherein at least one or more RNA substance may be at least partially embedded in or at least partially bound to or at least partially complexed with one or more RNA stabilizing substance, such as a stabilizing polymer as a non-limiting example.

In one embodiment one or more stabilizing complex may comprise at least one or more RNA stabilizing substance, such as a stabilizing polymer as a non-limiting example, and at least one or more RNA substance. In one embodiment one or more stabilizing complex may comprise two or more RNA stabilizing substances and at least one or more RNA substance. In one embodiment one or more stabilizing complex may comprise three or more RNA stabilizing substances and at least one or more RNA substance. In one embodiment one or more stabilizing complex may comprise four or more RNA stabilizing substances and at least one or more RNA substance. In one embodiment one or more stabilizing complex may comprise five or more RNA stabilizing substances and at least one or more RNA substance.

In other embodiments one or more stabilizing complex may comprise at least one or more RNA stabilizing substance, such as a stabilizing polymer as a non-limiting example, at least one or more RNA substance and one or more additional substance, such as a cellular uptake agent as a non-limiting example. Other embodiments may include compositions comprising one or more stabilizing complex wherein a stabilizing complex may comprise one or more RNA stabilizing substance, or two or more RNA stabilizing substances, or three or more RNA stabilizing substances, or four or more RNA stabilizing substances, or five or more RNA stabilizing substances, and at least one or more RNA substance and one or more additional substance, such as a cellular uptake agent as a non-limiting example.

Other embodiments of the present invention may include one or more composition described herein comprised of one or more RNA stabilizing substance and at least one or more RNA substance, wherein one or more composition may also comprise a stabilizing complex comprised of one or more RNA stabilizing substance and at least one or more RNA substance.

Other embodiments of the present invention may include one or more composition described herein comprised of one or more RNA stabilizing substance, at least one or more RNA substance, and one or more additional substance, such as one or more additional RNA stabilizing substance or one or more cellular uptake agent as non-limiting examples, wherein one or more composition may also comprise a stabilizing complex comprised of one or more RNA stabilizing substance, at least one or more RNA substance, and one or more additional substance, such as one or more additional RNA stabilizing substance or one or more cellular uptake agent as non-limiting examples.

For conciseness the following ranges of at least one stabilizing complex mean perimeter dimension, is herein referred to as the stabilizing complex mean perimeter dimension range list.

In some embodiments one or more stabilizing complex may have at least one dimension with a mean perimeter between about 10 nm-10 cm. In some embodiments one or more stabilizing complex may have at least one dimension with a mean perimeter between about 10 nm-1 cm. In some embodiments one or more stabilizing complex may have at least one dimension with a mean perimeter between about 10 nm-100 mm. In some embodiments one or more stabilizing complex may have at least one dimension with a mean perimeter between about 10 nm-10 mm. In some embodiments one or more stabilizing complex may have at least one dimension with a mean perimeter between about 10 nm-1 mm. In some embodiments one or more stabilizing complex may have at least one dimension with a mean perimeter between about 10 nm-100 m. In some embodiments one or more stabilizing complex may have at least one dimension with a mean perimeter between about 10 nm-10 μm. In some embodiments one or more stabilizing complex may have at least one dimension with a mean perimeter between about 10 nm-1 μm. In some embodiments one or more stabilizing complex may have at least one dimension with a mean perimeter between about 10 nm-500 nm. In some embodiments one or more stabilizing complex may have at least one dimension with a mean perimeter between about 10 nm-400 nm. In some embodiments one or more stabilizing complex may have at least one dimension with a mean perimeter between about 10 nm-300 nm. In some embodiments one or more stabilizing complex may have at least one dimension with a mean perimeter between about 10 nm-200 nm. In some embodiments one or more stabilizing complex may have at least one dimension with a mean perimeter between about 10 nm-100 nm.

In some embodiments one or more stabilizing complex may have at least one dimension with a mean perimeter between about 100 nm-10 cm. In some embodiments one or more stabilizing complex may have at least one dimension with a mean perimeter between about 100 nm-1 cm. In some embodiments one or more stabilizing complex may have at least one dimension with a mean perimeter between about 100 nm-100 mm. In some embodiments one or more stabilizing complex may have at least one dimension with a mean perimeter between about 100 nm-10 mm. In some embodiments one or more stabilizing complex may have at least one dimension with a mean perimeter between about 100 nm-1 mm. In some embodiments one or more stabilizing complex may have at least one dimension with a mean perimeter between about 100 nm-100 μm. In some embodiments one or more stabilizing complex may have at least one dimension with a mean perimeter between about 100 nm-10 μm. In some embodiments one or more stabilizing complex may have at least one dimension with a mean perimeter between about 100 nm-1 μm. In some embodiments one or more stabilizing complex may have at least one dimension with a mean perimeter between about 100 nm-500 nm. In some embodiments one or more stabilizing complex may have at least one dimension with a mean perimeter between about 100 nm-400 nm. In some embodiments one or more stabilizing complex may have at least one dimension with a mean perimeter between about 100 nm-300 nm. In some embodiments one or more stabilizing complex may have at least one dimension with a mean perimeter between about 100 nm-200 nm.

In some embodiments one or more stabilizing complex may have at least one dimension with a mean perimeter between about 200 nm-10 cm. In some embodiments one or more stabilizing complex may have at least one dimension with a mean perimeter between about 200 nm-1 cm. In some embodiments one or more stabilizing complex may have at least one dimension with a mean perimeter between about 200 nm-100 mm. In some embodiments one or more stabilizing complex may have at least one dimension with a mean perimeter between about 200 nm-10 mm. In some embodiments one or more stabilizing complex may have at least one dimension with a mean perimeter between about 200 nm-1 mm. In some embodiments one or more stabilizing complex may have at least one dimension with a mean perimeter between about 200 nm-100 μm. In some embodiments one or more stabilizing complex may have at least one dimension with a mean perimeter between about 200 nm-10 μm. In some embodiments one or more stabilizing complex may have at least one dimension with a mean perimeter between about 200 nm-1 μm. In some embodiments one or more stabilizing complex may have at least one dimension with a mean perimeter between about 200 nm-500 nm. In some embodiments one or more stabilizing complex may have at least one dimension with a mean perimeter between about 200 nm-400 nm. In some embodiments one or more stabilizing complex may have at least one dimension with a mean perimeter between about 200 nm-300 nm.

In some embodiments one or more stabilizing complex may have at least one dimension with a mean perimeter between about 300 nm-10 cm. In some embodiments one or more stabilizing complex may have at least one dimension with a mean perimeter between about 300 nm-1 cm. In some embodiments one or more stabilizing complex may have at least one dimension with a mean perimeter between about 300 nm-100 mm. In some embodiments one or more stabilizing complex may have at least one dimension with a mean perimeter between about 300 nm-10 mm. In some embodiments one or more stabilizing complex may have at least one dimension with a mean perimeter between about 300 nm-1 mm. In some embodiments one or more stabilizing complex may have at least one dimension with a mean perimeter between about 300 nm-100 μm. In some embodiments one or more stabilizing complex may have at least one dimension with a mean perimeter between about 300 nm-10 μm. In some embodiments one or more stabilizing complex may have at least one dimension with a mean perimeter between about 300 nm-1 μm. In some embodiments one or more stabilizing complex may have at least one dimension with a mean perimeter between about 300 nm-500 nm. In some embodiments one or more stabilizing complex may have at least one dimension with a mean perimeter between about 300 nm-400 nm.

In some embodiments one or more stabilizing complex may have at least one dimension with a mean perimeter between about 400 nm-10 cm. In some embodiments one or more stabilizing complex may have at least one dimension with a mean perimeter between about 400 nm-1 cm. In some embodiments one or more stabilizing complex may have at least one dimension with a mean perimeter between about 400 nm-100 mm. In some embodiments one or more stabilizing complex may have at least one dimension with a mean perimeter between about 400 nm-10 mm. In some embodiments one or more stabilizing complex may have at least one dimension with a mean perimeter between about 400 nm-1 mm. In some embodiments one or more stabilizing complex may have at least one dimension with a mean perimeter between about 400 nm-100 μm. In some embodiments one or more stabilizing complex may have at least one dimension with a mean perimeter between about 400 nm-10 μm. In some embodiments one or more stabilizing complex may have at least one dimension with a mean perimeter between about 400 nm-1 μm. In some embodiments one or more stabilizing complex may have at least one dimension with a mean perimeter between about 400 nm-500 nm.

In some embodiments one or more stabilizing complex may have at least one dimension with a mean perimeter between about 500 nm-10 cm. In some embodiments one or more stabilizing complex may have at least one dimension with a mean perimeter between about 500 nm-1 cm. In some embodiments one or more stabilizing complex may have at least one dimension with a mean perimeter between about 500 nm-100 mm. In some embodiments one or more stabilizing complex may have at least one dimension with a mean perimeter between about 500 nm-10 mm. In some embodiments one or more stabilizing complex may have at least one dimension with a mean perimeter between about 500 nm-1 mm. In some embodiments one or more stabilizing complex may have at least one dimension with a mean perimeter between about 500 nm-100 μm. In some embodiments one or more stabilizing complex may have at least one dimension with a mean perimeter between about 500 nm-10 μm. In some embodiments one or more stabilizing complex may have at least one dimension with a mean perimeter between about 500 nm-1 μm.

In some embodiments one or more stabilizing complex may have at least one dimension with a mean perimeter between about 1 μm-10 cm. In some embodiments one or more stabilizing complex may have at least one dimension with a mean perimeter between about 1 μm-1 cm. In some embodiments one or more stabilizing complex may have at least one dimension with a mean perimeter between about 1 μm-100 mm. In some embodiments one or more stabilizing complex may have at least one dimension with a mean perimeter between about 1 μm-10 mm. In some embodiments one or more stabilizing complex may have at least one dimension with a mean perimeter between about 1 μm-1 mm. In some embodiments one or more stabilizing complex may have at least one dimension with a mean perimeter between about 1 μm-100 μm. In some embodiments one or more stabilizing complex may have at least one dimension with a mean perimeter between about 1 μm-10 μm.

In some embodiments one or more stabilizing complex may have at least one dimension with a mean perimeter between about 10 μm-10 cm. In some embodiments one or more stabilizing complex may have at least one dimension with a mean perimeter between about 10 μm-1 cm. In some embodiments one or more stabilizing complex may have at least one dimension with a mean perimeter between about 10 μm-100 mm. In some embodiments one or more stabilizing complex may have at least one dimension with a mean perimeter between about 10 μm-10 mm. In some embodiments one or more stabilizing complex may have at least one dimension with a mean perimeter between about 10 μm-1 mm. In some embodiments one or more stabilizing complex may have at least one dimension with a mean perimeter between about 10 μm-100 μm.

In some embodiments one or more stabilizing complex may have at least one dimension with a mean perimeter between about 100 μm-10 cm. In some embodiments one or more stabilizing complex may have at least one dimension with a mean perimeter between about 100 μm-1 cm. In some embodiments one or more stabilizing complex may have at least one dimension with a mean perimeter between about 100 μm-100 mm. In some embodiments one or more stabilizing complex may have at least one dimension with a mean perimeter between about 100 μm-10 mm. In some embodiments one or more stabilizing complex may have at least one dimension with a mean perimeter between about 100 μm-1 mm.

In some embodiments one or more stabilizing complex may have at least one dimension with a mean perimeter between about 1 mm-10 cm. In some embodiments one or more stabilizing complex may have at least one dimension with a mean perimeter between about 1 mm-1 cm. In some embodiments one or more stabilizing complex may have at least one dimension with a mean perimeter between about 1 mm-100 mm. In some embodiments one or more stabilizing complex may have at least one dimension with a mean perimeter between about 1 mm-10 mm.

In some embodiments one or more stabilizing complex may have at least one dimension with a mean perimeter between about 100 nm-10 cm. In some embodiments one or more stabilizing complex may have at least one dimension with a mean perimeter between about 100 nm-1 cm. In some embodiments one or more stabilizing complex may have at least one dimension with a mean perimeter between about 100 nm-100 mm. In some embodiments one or more stabilizing complex may have at least one dimension with a mean perimeter between about 100 nm-10 mm. In some embodiments one or more stabilizing complex may have at least one dimension with a mean perimeter between about 100 nm-1 mm. In some embodiments one or more stabilizing complex may have at least one dimension with a mean perimeter between about 100 nm-100m. In some embodiments one or more stabilizing complex may have at least one dimension with a mean perimeter between about 100 nm-10 μm. In some embodiments one or more stabilizing complex may have at least one dimension with a mean perimeter between about 100 nm-1 μm. In some embodiments one or more stabilizing complex may have at least one dimension with a mean perimeter between about 100 nm-500 nm. In some embodiments one or more stabilizing complex may have at least one dimension with a mean perimeter between about 100 nm-400 nm. In some embodiments one or more stabilizing complex may have at least one dimension with a mean perimeter between about 100 nm-300 nm. In some embodiments one or more stabilizing complex may have at least one dimension with a mean perimeter between about 100 nm-200 nm.

For conciseness the following list of at least one stabilizing complex mean perimeter dimension, is herein referred to as the stabilizing complex mean perimeter dimension list.

In some embodiments one or more stabilizing complex may have at least one dimension with a mean perimeter greater than about 1 nm. In some embodiments one or more stabilizing complex may have at least one dimension with a mean perimeter greater than about 10 nm. In some embodiments one or more stabilizing complex may have at least one dimension with a mean perimeter greater than about 50 nm. In some embodiments one or more stabilizing complex may have at least one dimension with a mean perimeter greater than about 100 nm. In some embodiments one or more stabilizing complex may have at least one dimension with a mean perimeter greater than about 200 nm. In some embodiments one or more stabilizing complex may have at least one dimension with a mean perimeter greater than about 300 nm. In some embodiments one or more stabilizing complex may have at least one dimension with a mean perimeter greater than about 400 nm. In some embodiments one or more stabilizing complex may have at least one dimension with a mean perimeter greater than about 500 nm. In some embodiments one or more stabilizing complex may have at least one dimension with a mean perimeter greater than about 600 nm. In some embodiments one or more stabilizing complex may have at least one dimension with a mean perimeter greater than about 700 nm. In some embodiments one or more stabilizing complex may have at least one dimension with a mean perimeter greater than about 800 nm. In some embodiments one or more stabilizing complex may have at least one dimension with a mean perimeter greater than about 900 nm. In some embodiments one or more stabilizing complex may have at least one dimension with a mean perimeter greater than about 1 μm. In some embodiments one or more stabilizing complex may have at least one dimension with a mean perimeter greater than about 2 μm. In some embodiments one or more stabilizing complex may have at least one dimension with a mean perimeter greater than about 3 μm. In some embodiments one or more stabilizing complex may have at least one dimension with a mean perimeter greater than about 4 μm. In some embodiments one or more stabilizing complex may have at least one dimension with a mean perimeter greater than about 5 μm. In some embodiments one or more stabilizing complex may have at least one dimension with a mean perimeter greater than about 6 μm. In some embodiments one or more stabilizing complex may have at least one dimension with a mean perimeter greater than about 7 μm. In some embodiments one or more stabilizing complex may have at least one dimension with a mean perimeter greater than about 8 μm. In some embodiments one or more stabilizing complex may have at least one dimension with a mean perimeter greater than about 9 μm. In some embodiments one or more stabilizing complex may have at least one dimension with a mean perimeter greater than about 10 μm. In some embodiments one or more stabilizing complex may have at least one dimension with a mean perimeter greater than about 20 μm. In some embodiments one or more stabilizing complex may have at least one dimension with a mean perimeter greater than about 30 μm. In some embodiments one or more stabilizing complex may have at least one dimension with a mean perimeter greater than about 40 μm. In some embodiments one or more stabilizing complex may have at least one dimension with a mean perimeter greater than about 50 μm. In some embodiments one or more stabilizing complex may have at least one dimension with a mean perimeter greater than about 60 μm. In some embodiments one or more stabilizing complex may have at least one dimension with a mean perimeter greater than about 70 μm. In some embodiments one or more stabilizing complex may have at least one dimension with a mean perimeter greater than about 80 μm. In some embodiments one or more stabilizing complex may have at least one dimension with a mean perimeter greater than about 90 μm. In some embodiments one or more stabilizing complex may have at least one dimension with a mean perimeter greater than about 100 μm. In some embodiments one or more stabilizing complex may have at least one dimension with a mean perimeter greater than about 200 μm. In some embodiments one or more stabilizing complex may have at least one dimension with a mean perimeter greater than about 300 μm. In some embodiments one or more stabilizing complex may have at least one dimension with a mean perimeter greater than about 400 μm. In some embodiments one or more stabilizing complex may have at least one dimension with a mean perimeter greater than about 500 μm. In some embodiments one or more stabilizing complex may have at least one dimension with a mean perimeter greater than about 600 μm. In some embodiments one or more stabilizing complex may have at least one dimension with a mean perimeter greater than about 700 μm. In some embodiments one or more stabilizing complex may have at least one dimension with a mean perimeter greater than about 800 μm. In some embodiments one or more stabilizing complex may have at least one dimension with a mean perimeter greater than about 900 μm. In some embodiments one or more stabilizing complex may have at least one dimension with a mean perimeter greater than about 1 mm. In some embodiments one or more stabilizing complex may have at least one dimension with a mean perimeter greater than about 2 mm. In some embodiments one or more stabilizing complex may have at least one dimension with a mean perimeter greater than about 3 mm. In some embodiments one or more stabilizing complex may have at least one dimension with a mean perimeter greater than about 4 mm. In some embodiments one or more stabilizing complex may have at least one dimension with a mean perimeter greater than about 5 mm. In some embodiments one or more stabilizing complex may have at least one dimension with a mean perimeter greater than about 10 mm. In some embodiments one or more stabilizing complex may have at least one dimension with a mean perimeter greater than about 20 mm. In some embodiments one or more stabilizing complex may have at least one dimension with a mean perimeter greater than about 50 mm. In some embodiments one or more stabilizing complex may have at least one dimension with a mean perimeter greater than about 100 mm. In some embodiments one or more stabilizing complex may have at least one dimension with a mean perimeter greater than about 200 mm. In some embodiments one or more stabilizing complex may have at least one dimension with a mean perimeter greater than about 500 mm. In some embodiments one or more stabilizing complex may have at least one dimension with a mean perimeter greater than about 1 cm.

In some embodiments the mean perimeter of the stabilizing complex may be a hydrodynamic mean perimeter. In some embodiments the mean perimeter may be measured with transmission electron microscopy (TEM), or with dynamic light scattering (DLS), or other known methods in the art.

One embodiment of the present invention is the method whereby one or more stabilizing polymer may be combined, such as by mixing, with at least one or more RNA substance to produce a mixture comprising at least one or more stabilizing polymer and at least one or more RNA substance. As a non-limiting example one or more RNA substance may be mixed with one or more stabilizing polymer.

Another embodiment of the present invention is the method whereby one or more stabilizing polymer may be combined, such as by mixing, with at least one or more RNA substance to produce a mixture comprising at least one or more RNA substance and at least one or more stabilizing polymer at one or more of the RNA stabilizing substance concentrations within the stabilizing substance molar concentration range list, or in the stabilizing substance molar concentration list, or in the stabilizing substance weight percent concentration list, or in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. These same methods may be used to combine one or more stabilizing polymer with one or more RNA substance at one or more ratio in the stabilizing polymer C/P ratio list, or in the stabilizing polymer N/P ratio list, or in the stabilizing polymer C/P ratio range list, or in the stabilizing polymer N/P ratio range list. These same methods may be used to combine one or more other substances, including, but not limited to one or more cellular uptake agents or one or more additional RNA stabilizing substances, with one or more RNA substances and one or more stabilizing polymers to produce a mixture comprising one or more RNA substance, one or more stabilizing polymer, and one or more cellular uptake agent or one or more additional RNA stabilizing substance.

One embodiment of the present invention is the method whereby one or more stabilizing polymer may be combined, such as by mixing, with at least one or more RNA substance to produce a composition comprising at least one or more RNA substance and at least one or more stabilizing polymer. Another embodiment of the present invention is the method whereby one or more stabilizing polymer may be combined, such as by mixing, with one or more RNA substance to produce a composition comprising at least one or more RNA substance and at least one or more stabilizing polymer at one or more of the RNA stabilizing substance concentrations within the stabilizing substance molar concentration range list, or in the stabilizing substance molar concentration list, or in the or stabilizing substance weight percent concentration list, or in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. These same methods may be used to combine one or more stabilizing polymer with one or more RNA substance at one or more ratio in the stabilizing polymer C/P ratio list, or in the stabilizing polymer N/P ratio list, or in the stabilizing polymer C/P ratio range list, or in the stabilizing polymer N/P ratio range list. These same methods may be used to combine one or more other substances, including, but not limited to one or more cellular uptake agents or one or more additional RNA stabilizing substance, with one or more RNA substance and one or more stabilizing polymer to produce a composition comprising one or more RNA substance, one or more stabilizing polymer, and one or more cellular uptake agent or one or more additional RNA stabilizing substance.

In one embodiment a stabilizing polymer may be anionic. In one embodiment a stabilizing polymer may be cationic. In one embodiment a stabilizing may be zwitterionic. In one embodiment a stabilizing polymer may comprise one or more cationic moiety. In one embodiment a stabilizing polymer may comprise one or more anionic moiety. In one embodiment a stabilizing polymer may comprise one or more zwitterionic moiety.

In one embodiment a stabilizing polymer may comprise one or more PIF as described herein.

In one embodiment one or more composition comprising one or more RNA substance and one or more stabilizing polymer may comprise one or more water concentrations in the water concentration list or in the water concentration range list. In one embodiment one or more composition comprising one or more RNA substance, one or more stabilizing polymer, and one or more additional substance, such as one or more cellular uptake agent or one or more additional RNA stabilizing substance as non-limiting examples, may comprise one or more water concentrations in the water concentration list or in the water concentration range list. In one embodiment one or more composition comprising one or more RNA substance and one or more stabilizing polymer may have a dielectric constant of one or more dielectric constant in the dielectric constant list or in the dielectric constant range list. In one embodiment one or more composition comprising one or more RNA substance, one or more stabilizing polymer, and one or more additional substance, such as one or more cellular uptake agent or one or more additional RNA stabilizing substance as non-limiting examples, may have a dielectric constant of one or more dielectric constant in the dielectric constant list or in the dielectric constant range list.

Embodiments of the present invention may include compositions comprising one or more stabilizing polymer used in a composition comprising one or more RNA substance, one or more stabilizing polymer, and one or more of the following substances: water, cellular uptake agents, aprotic substances, low dielectric substances, cyclic phosphate or metaphosphate containing substances, non-carbohydrate organic osmolyte substances, tertiary sulfonium containing substances, quaternary ammonium containing substances, quaternary phosphonium containing substances, hydrotrope containing substances, surfactant containing substances, betaine containing substances, stabilizing monomers, or supplemental RNA stabilizing substances. Other embodiments may include one or more composition comprising one or more stabilizing polymer and one or more RNA substance and may also comprise one or more cellular uptake agent or one or more additional RNA stabilizing substances. Other embodiments may include compositions comprising one or more stabilizing polymer, one or more RNA substance, and one or more additional substances, such as: one or more cellular uptake agent, or one or more additional RNA stabilizing substance, or one or more buffering agent, or one or more chelating agent, or one or more inorganic salt, or water, as non-limiting examples.

Embodiments of the present invention may include one or more compositions comprising one or more RNA substance and one or more stabilizing polymer, wherein a composition is not lyophilized.

In one embodiment one or more composition comprising one or more RNA substance and one or more stabilizing polymer may not be lyophilized. In one embodiment one or more composition comprising one or more RNA substance, one or more stabilizing polymer, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more stabilizing polymer, wherein a composition may be lyophilized.

In one embodiment one or more composition comprising one or more RNA substance and one or more stabilizing polymer may be lyophilized. In one embodiment one or more composition comprising one or more RNA substance, one or more stabilizing polymer, and one or more additional substance, such as one or more cellular uptake agent or one or more additional RNA stabilizing substance as non-limiting examples, may be lyophilized.

In one embodiment a stabilizing polymer may not comprise a lipid. In one embodiment a stabilizing polymer may not comprise a cholesterol. In one embodiment a stabilizing polymer is not a saccharide, polysaccharide, or starch.

In one embodiment a stabilizing polymer may not comprise a detergent. In one embodiment a stabilizing polymer may not comprise a peptide or polypeptide. In one embodiment a stabilizing polymer may not be a polypeptide or protein. In one embodiment a stabilizing polymer may not comprise a nucleic acid base. In one embodiment a stabilizing polymer may not comprise a nucleic acid. In one embodiment a stabilizing polymer may not be a polynucleotide or poly nucleic acid.

Embodiments of the present invention may include one or more compositions comprising one or more stabilizing polymer and one or more RNA substance described herein, wherein one or more composition described herein may have a melting of one or more melting point in the stabilizing composition melting point list.

In one embodiment one or more composition comprising one or more RNA substance and one or more stabilizing polymer may have a melting point of one or more melting point in the stabilizing composition melting point list. In one embodiment one or more composition comprising one or more RNA substance, one or more stabilizing polymer, and one or more additional substance, such as one or more cellular uptake agent or one or more additional RNA stabilizing substance as a non-limiting example, may have a melting point of one or more melting point in the stabilizing composition melting point list.

### Supplemental RNA Stabilizing Substances:

The inventors have discovered that supplemental RNA stabilizing substances may stabilize RNA substances.

The inventors have discovered that RNA stabilizing substances may comprise supplemental RNA stabilizing substances. The inventors have also discovered that the stability of RNA substances may be enhanced in an RNA storage environment comprising a supplemental RNA stabilizing substance. The inventors have also discovered that the stability of RNA substances may be enhanced in an RNA storage environment comprising a supplemental RNA stabilizing substance and one or more additional RNA stabilizing substance, such as a stabilizing polymer.

The inventors have also discovered that the stability of RNA substances may be enhanced in compositions comprising an RNA substance and a supplemental RNA stabilizing substance. The inventors have also discovered that the stability of RNA substances may be enhanced in compositions comprising an RNA substance, a supplemental RNA stabilizing substance, and one or more additional RNA stabilizing substance, such as a stabilizing polymer.

In one embodiment of the present invention, one or more RNA stabilizing substance may comprise a supplemental RNA stabilizing substance.

One embodiment of the present invention may include a composition comprised of one or more RNA substance and one or more supplemental RNA stabilizing substance. Another embodiment of the present invention may include a composition comprised of one or more RNA substance, one or more supplemental RNA stabilizing substance, and one or more additional RNA stabilizing substance, such as a stabilizing polymer.

One embodiment of the present invention may include a combination or mixture comprising one or more RNA substance and one or more supplemental RNA stabilizing substance. Another embodiment of the present invention may include a combination or mixture comprising one or more RNA substance, one or more supplemental RNA stabilizing substance, and one or more additional RNA stabilizing substance, such as an stabilizing polymer.

Embodiments of the present invention that comprise one or more RNA substance and one or more supplemental RNA stabilizing substance may include combining, such as by mixing, one or more RNA substance with one or more substance that comprises at least one or more supplemental RNA stabilizing substance.

Embodiments of the present invention that comprise one or more RNA substance, one or more supplemental RNA stabilizing substance, and one or more additional RNA stabilizing substance, such as an stabilizing polymer, may include combining, such as by mixing, one or more RNA substance with one or more substance that comprises at least one or more supplemental RNA stabilizing substance and one or more substance that comprises at least one or more RNA stabilizing substance, such as an stabilizing polymer.

An embodiment of the present invention may include compositions of materials that comprise one or more RNA substance and at least one or more supplemental RNA stabilizing substance. The storage environment that improves the stability of RNA substances may comprise at least one or more vapor, liquid, powder, or solid supplemental RNA stabilizing substance.

An embodiment of the present invention may include compositions of materials that comprise one or more RNA substance, one or more supplemental RNA stabilizing substance, and one or more additional RNA stabilizing substance, such as an stabilizing polymer. The storage environment that improves the stability of RNA substances may comprise at least one or more vapor, liquid, powder, or solid supplemental RNA stabilizing substance and may comprise at least one or more vapor, liquid, powder, or solid RNA stabilizing substance, such as an stabilizing polymer.

An embodiment of the present invention may include combinations of materials that comprise one or more RNA substance and at least one or more supplemental RNA stabilizing substance. The storage environment that improves the stability of RNA substances may comprise at least one or more vapor, liquid, powder, or solid supplemental RNA stabilizing substance.

An embodiment of the present invention may include combinations of materials that comprise one or more RNA substance, one or more supplemental RNA stabilizing substance, and one or more additional RNA stabilizing substance, such as an stabilizing polymer. The storage environment that improves the stability of RNA substances may comprise at least one or more vapor, liquid, powder, or solid supplemental RNA stabilizing substance and may comprise at least one or more vapor, liquid, powder, or solid RNA stabilizing substance, such as a stabilizing polymer.

In one embodiment of the present invention a composition comprised of one or more RNA substance and one or more supplemental RNA stabilizing substance, produces a mixture with at least one or more RNA substance and at least one or more supplemental RNA stabilizing substance. In one embodiment of the present invention a composition comprised of one or more RNA substance, one or more supplemental RNA stabilizing substance, and one or more additional RNA stabilizing substance, such as an stabilizing polymer, produces a mixture with at least one or more RNA substance, at least one or more supplemental RNA stabilizing substance, and at least one or more additional RNA stabilizing substance, such as a stabilizing polymer.

In one embodiment of the present invention a combination comprising one or more RNA substance and one or more supplemental RNA stabilizing substance, produces a mixture with at least one or more RNA substance and at least one or more supplemental RNA stabilizing substance. In one embodiment of the present invention a combination comprising one or more RNA substance, one or more supplemental RNA stabilizing substance, and one or more additional RNA stabilizing substance, such as an stabilizing polymer, produces a mixture with at least one or more RNA substance, at least one or more supplemental RNA stabilizing substance, and at least one or more additional RNA stabilizing substance, such as a stabilizing polymer.

In one embodiment of the present invention a composition with improved RNA stability may comprise one or more RNA substance and one or more supplemental RNA stabilizing substance. In one embodiment of the present invention a composition with improved RNA stability may comprise one or more RNA substance, one or more supplemental RNA stabilizing substance, and one or more additional RNA stabilizing substance, such as a stabilizing polymer.

In one embodiment of the present invention a composition with improved RNA stability may comprise a mixture of one or more RNA substance and one or more supplemental RNA stabilizing substance. In one embodiment of the present invention a composition with improved RNA stability may comprise a mixture of one or more RNA substance, one or more supplemental RNA stabilizing substance, and one or more additional RNA stabilizing substance, such as a stabilizing polymer.

In one embodiment of the present invention, each component included in a composition comprising one or more RNA substance and one or more supplemental RNA stabilizing substance, may be stored separately, such as in a kit, or such as individual substances or as mixtures of one or more substance, and then combined later to produce a composition comprising one or more RNA substance and one or more supplemental RNA stabilizing substance.

In one embodiment of the present invention, each component included in a composition comprising one or more RNA substance, one or more supplemental RNA stabilizing substance, and one or more additional RNA stabilizing substance, such as an stabilizing polymer, may be stored separately, such as in a kit, or such as individually or as mixtures of one or more substance, and then combined later to produce a composition comprising one or more RNA substance, one or more supplemental RNA stabilizing substance, and one or more additional RNA stabilizing substance, such as a stabilizing polymer.

In one embodiment of the present invention a composition comprising one or more RNA substance and one or more supplemental RNA stabilizing substance may be at least partially biocompatible.

In one embodiment of the present invention a composition comprising one or more RNA substance, one or more supplemental RNA stabilizing substance, and one or more additional RNA stabilizing substance, such as a stabilizing polymer, may be at least partially biocompatible.

In one embodiment of the present invention a combination comprised of one or more RNA substance and one or more supplemental RNA stabilizing substance may be at least partially biocompatible.

In one embodiment of the present invention a combination comprised of one or more RNA substance, one or more supplemental RNA stabilizing substance, and one or more additional RNA stabilizing substance, such as a stabilizing polymer, may be at least partially biocompatible.

Embodiments of the present invention comprising at least one or more supplemental RNA stabilizing substance may include one or more forms of the substance. These forms may include, but are not limited to, one more counterions, ionic forms, conjugate bases, conjugate acids, protonated or deprotonated forms, isomers, structural isomers, stereo isomers, chiral forms, salts, or combinations thereof.

In one embodiment one or more supplemental RNA stabilizing substance may comprise a polyether, such as polyethylene glycol or polypropylene glycol as non-limiting examples.

In one embodiment a supplemental RNA stabilizing substance, such as a polyether as a non-limiting example, may comprise one or more ether bond. In one embodiment a supplemental RNA stabilizing substance, such as a polyether as a non-limiting example, may comprise 1 or more ether bonds, 2 or more ether bonds, 3 or more ether bonds, 4 or more ether bonds, 5 or more ether bonds, 10 or more ether bonds, or 50 or more ether bonds. In one embodiment a supplemental RNA stabilizing substance, such as a polyether as a non-limiting example, may comprise between about 1-1,000 ether bonds, or between about 1-500 ether bonds, or between about 1-100 ether bonds, or between about 1-50 ether bonds, or between about 1-20 ether bonds, or between about 1-10 ether bonds, or between about 2-1,000 ether bonds, or between about 2-500 ether bonds, or between about 2-100 ether bonds, or between about 2-50 ether bonds, or between about 2-20 ether bonds, or between about 2-10 ether bonds.

In one embodiment a supplemental RNA stabilizing substance, such as a polyether as a non-limiting example, may comprise one or more ether. In one embodiment a supplemental RNA stabilizing substance, such as a polyether as a non-limiting example, may comprise 1 or more ethers, 2 or more ethers, 3 or more ethers, 4 or more ethers, 5 or more ethers, 10 or more ethers, or 50 or more ethers. In one embodiment a supplemental RNA stabilizing substance, such as a polyether as a non-limiting example, may comprise between about 1-1,000 ethers, or between about 1-500 ethers, or between about 1-100 ethers, or between about 1-50 ethers, or between about 1-20 ethers, or between about 1-10 ethers, or between about 2-1,000 ethers, or between about 2-500 ethers, or between about 2-100 ethers, or between about 2-50 ethers, or between about 2-20 ethers, or between about 2-10 ethers.

A non-limiting example of a supplemental RNA stabilizing substance that may be used is polyethylene glycol (PEG), such as PEG (˜8 kDa) as a non-limiting example, wherein PEG may be substituted for or used in combination with one or more supplemental RNA stabilizing substances described herein. One embodiment of the present invention may be a combination of substances comprising PEG and another embodiment may also comprise at least one RNA substance. Other embodiments may be combinations of substances comprising PEG wherein the concentration of PEG may be one or more concentration in the stabilizing substance weight percent concentration list or in the stabilizing substance molar concentration range list or in the stabilizing substance molar concentration list.

A non-limiting example of a supplemental RNA stabilizing substance that may be used is polypropylene glycol (PPG), such as PPG (˜425 kDa) as a non-limiting example, wherein PPG may be substituted for or used in combination with one or more supplemental RNA stabilizing substance as described herein. One embodiment of the present invention may be a combination of substances comprising PPG and another embodiment may also comprise at least one RNA substance. Other embodiments may be combinations of substances comprising PPG wherein the concentration of PPG may be one or more concentration in the stabilizing substance weight percent concentration list or in the stabilizing substance molar concentration range list or in the stabilizing substance molar concentration list.

A non-limiting example of a supplemental RNA stabilizing substance that may be used is glycerol (also known as glycerin), wherein glycerol may be substituted for or used in combination with one or more supplemental RNA stabilizing substance as described herein. One embodiment of the present invention may be a combination of substances comprising glycerol another embodiment may also comprise at least one RNA substance. Other embodiments may be combinations of substances comprising glycerol wherein the concentration of glycerol may be one or more concentration in the stabilizing substance weight percent concentration list or in the stabilizing substance molar concentration range list or in the stabilizing substance molar concentration list.

A non-limiting example of a supplemental RNA stabilizing substance that may be used is glycerol phosphate, such as glycerol phosphate disodium salt as a non-limiting example, wherein glycerol phosphate may be substituted for or used in combination with one or more supplemental RNA stabilizing substance as described herein. One embodiment of the present invention may be a combination of substances comprising glycerol phosphate another embodiment may also comprise at least one RNA substance. Other embodiments may be combinations of substances comprising glycerol phosphate wherein the concentration of glycerol phosphate may be one or more concentration in the stabilizing substance weight percent concentration list or in the stabilizing substance molar concentration range list or in the stabilizing substance molar concentration list.

A non-limiting example of a supplemental RNA stabilizing substance that may be used is diglycerol phosphate, wherein diglycerol phosphate may be substituted for or used in combination with one or more supplemental RNA stabilizing substance as described herein. One embodiment of the present invention may be a combination of substances comprising diglycerol phosphate another embodiment may also comprise at least one RNA substance. Other embodiments may be combinations of substances comprising diglycerol phosphate wherein the concentration of diglycerol phosphate may be one or more concentration in the stabilizing substance weight percent concentration list or in the stabilizing substance molar concentration range list or in the stabilizing substance molar concentration list.

A non-limiting example of a supplemental RNA stabilizing substance that may be used is hexylene glycol (also known as 2-methylpentane-2,4-diol, MPD, or diolane), wherein hexylene glycol may be substituted for or used in combination with one or more supplemental RNA stabilizing substance as described herein. One embodiment of the present invention may be a combination of substances comprising hexylene glycol another embodiment may also comprise at least one RNA substance. Other embodiments may be combinations of substances comprising hexylene glycol wherein the concentration of hexylene glycol may be one or more concentration in the stabilizing substance weight percent concentration list or in the stabilizing substance molar concentration range list or in the stabilizing substance molar concentration list.

A non-limiting example of a supplemental RNA stabilizing substance that may be used is poly(2-hydroxyethyl methacrylate) (also known as poly(2-HEMA) or poly-HEMA), such as poly-HEMA (˜20 kDa) as a non-limiting example, wherein poly-HEMA may be substituted for or used in combination with one or more supplemental RNA stabilizing substance as described herein. One embodiment of the present invention may be a combination of substances comprising poly-HEMA another embodiment may also comprise at least one RNA substance. Other embodiments may be combinations of substances comprising poly-HEMA wherein the concentration of poly-HEMA may be one or more concentration in the stabilizing substance weight percent concentration list or in the stabilizing substance molar concentration range list or in the stabilizing substance molar concentration list.

In one embodiment a supplemental RNA stabilizing substance may comprise one or more methylene phosphonic acid or methylene phosphonate, such as diethylenetriamine penta(methylene phosphonic acid) (DTPMP) or aminotris(methylenephosphonic acid) (ATMP), as non-limiting examples.

In one embodiment a supplemental RNA stabilizing substance, such as a methylene phosphonic acid or methyl phosphonate as non-limiting examples, may comprise one or more ether. In one embodiment a supplemental RNA stabilizing substance, such as a methylene phosphonic acid or methyl phosphonate as non-limiting examples, may comprise 1 or more phosphonic acids or phosphonates, 2 or more phosphonic acids or phosphonates, 3 or more phosphonic acids or phosphonates, 4 or more phosphonic acids or phosphonates, 5 or more phosphonic acids or phosphonates, 10 or more phosphonic acids or phosphonates, or 50 or more phosphonic acids or phosphonates. In one embodiment a supplemental RNA stabilizing substance, such as a methylene phosphonic acid or methyl phosphonate as non-limiting examples, may comprise between about 1-100 phosphonic acids or phosphonates, or between about 1-50 phosphonic acids or phosphonates, or between about 1-20 phosphonic acids or phosphonates, or between about 1-10 phosphonic acids or phosphonates, or between about 1-5 phosphonic acids or phosphonates, or between about 1-3 phosphonic acids or phosphonates, or between about 2-100 phosphonic acids or phosphonates, or between about 2-50 phosphonic acids or phosphonates, or between about 2-20 phosphonic acids or phosphonates, or between about 2-10 phosphonic acids or phosphonates, or between about 2-5 phosphonic acids or phosphonates, or between about 2-3 phosphonic acids or phosphonates.

In one embodiment a supplemental RNA stabilizing substance, such as a methylene phosphonic acid or methyl phosphonate as non-limiting examples, may comprise one or more ether. In one embodiment a supplemental RNA stabilizing substance, such as a methylene phosphonic acid or methyl phosphonate as non-limiting examples, may comprise 1 or more methylene phosphonic acids or methylene phosphonates, 2 or more methylene phosphonic acids or methylene phosphonates, 3 or more methylene phosphonic acids or methylene phosphonates, 4 or more methylene phosphonic acids or methylene phosphonates, 5 or more methylene phosphonic acids or methylene phosphonates, 10 or more methylene phosphonic acids or methylene phosphonates, or 50 or more methylene phosphonic acids or methylene phosphonates. In one embodiment a supplemental RNA stabilizing substance, such as a methylene phosphonic acid or methyl phosphonate as non-limiting examples, may comprise between about 1-100 methylene phosphonic acids or methylene phosphonates, or between about 1-50 methylene phosphonic acids or methylene phosphonates, or between about 1-20 methylene phosphonic acids or methylene phosphonates, or between about 1-10 methylene phosphonic acids or methylene phosphonates, or between about 1-5 methylene phosphonic acids or methylene phosphonates, or between about 1-3 methylene phosphonic acids or methylene phosphonates, or between about 2-100 methylene phosphonic acids or methylene phosphonates, or between about 2-50 methylene phosphonic acids or methylene phosphonates, or between about 2-20 methylene phosphonic acids or methylene phosphonates, or between about 2-10 methylene phosphonic acids or methylene phosphonates, or between about 2-5 methylene phosphonic acids or methylene phosphonates, or between about 2-3 methylene phosphonic acids or methylene phosphonates.

A non-limiting example of a supplemental RNA stabilizing substance comprised of a methylene phosphonic acid or methylene phosphonate that may be used is DTPMP, wherein DTPMP may be substituted for or used in combination with one or more supplemental RNA stabilizing substance as described herein. One embodiment of the present invention may be a combination of substances comprising DTPMP another embodiment may also comprise at least one RNA substance. Other embodiments may be combinations of substances comprising DTPMP wherein the concentration of DTPMP may be one or more concentration in the stabilizing substance weight percent concentration list or in the stabilizing substance molar concentration range list or in the stabilizing substance molar concentration list.

A non-limiting example of a supplemental RNA stabilizing substance comprised of a methylene phosphonic acid or methylene phosphonate that may be used is ATMP, wherein ATMP may be substituted for or used in combination with one or more supplemental RNA stabilizing substance as described herein. One embodiment of the present invention may be a combination of substances comprising ATMP another embodiment may also comprise at least one RNA substance. Other embodiments may be combinations of substances comprising ATMP wherein the concentration of ATMP may be one or more concentration in the stabilizing substance weight percent concentration list or in the stabilizing substance molar concentration range list or in the stabilizing substance molar concentration list.

For conciseness, the following list of supplemental RNA stabilizing substances is herein referred to as the supplemental RNA stabilizing substance list wherein one or more of the following substances may be substituted for or used in combination with one or more supplemental RNA stabilizing substance as described herein.

In other embodiments, other supplemental RNA stabilizing substances that may be substituted for or used in combination with one or more supplemental RNA stabilizing substances as described herein may comprise one or more of the following, including but not limited to: glycerol, polyether, polyethylene oxide, polyethylene glycol (PEG), polypropylene glycol (PPG), glycerol phosphate, diglycerol phosphate, methylpentane-2,4-diol, hexylene glycol, poly-HEMA, methylene phosphonic acid, methylene phosphonate, DTPMP, or ATMP, or salts, or combinations, or derivatives thereof.

Embodiments of the present invention may include combinations comprising one or more of the substances from the supplemental RNA stabilizing substance list substituted for or used in combination with one or more supplemental RNA stabilizing substance or one or more RNA stabilizing substance, such as a stabilizing polymer as a non-limiting example, and other embodiments may also comprise at least one RNA substance.

Other embodiments may include combinations of substances comprising one or more supplemental RNA stabilizing substance selected from the supplemental RNA stabilizing substance list wherein the concentration of one or more substances may be one or more concentration in the stabilizing substance weight percent concentration list or in the stabilizing substance molar concentration range list or in the stabilizing substance molar concentration list.

In one embodiment, a supplemental RNA stabilizing substance may have a molecular weight between about 100-1,000,000,000 daltons. In one embodiment, a supplemental RNA stabilizing substance may have a molecular weight between about 100-100,000,000 daltons. In one embodiment, a supplemental RNA stabilizing substance may have a molecular weight between about 100-10,000,000 daltons. In one embodiment, a supplemental RNA stabilizing substance may have a molecular weight between about 100-1,000,000 daltons. In one embodiment, a supplemental RNA stabilizing substance may have a molecular weight between about 100-500,000 daltons. In one embodiment, a supplemental RNA stabilizing substance may have a molecular weight between about 100-200,000 daltons. In one embodiment, a supplemental RNA stabilizing substance may have a molecular weight between about 100-100,000 daltons. In one embodiment, a supplemental RNA stabilizing substance may have a molecular weight between about 100-50,000 daltons. In one embodiment, a supplemental RNA stabilizing substance may have a molecular weight between about 100-20,000 daltons. In one embodiment, a supplemental RNA stabilizing substance may have a molecular weight between about 100-10,000 daltons. In one embodiment, a supplemental RNA stabilizing substance may have a molecular weight between about 100-5,000 daltons. In one embodiment, a supplemental RNA stabilizing substance may have a molecular weight between about 100-5,000 daltons. In one embodiment, a supplemental RNA stabilizing substance may have a molecular weight between about 100-2,000 daltons. In one embodiment, a supplemental RNA stabilizing substance may have a molecular weight between about 100-1,000 daltons. In one embodiment, a supplemental RNA stabilizing substance may have a molecular weight between about 100-900 daltons. In one embodiment, a supplemental RNA stabilizing substance may have a molecular weight between about 100-800 daltons. In one embodiment, a supplemental RNA stabilizing substance may have a molecular weight between about 100-700 daltons. In one embodiment, a supplemental RNA stabilizing substance may have a molecular weight between about 100-600 daltons. In one embodiment, a supplemental RNA stabilizing substance may have a molecular weight between about 100-500 daltons.

In one embodiment, a supplemental RNA stabilizing substance may have a molecular weight between about 200-1,000,000,000 daltons. In one embodiment, a supplemental RNA stabilizing substance may have a molecular weight between about 200-100,000,000 daltons. In one embodiment, a supplemental RNA stabilizing substance may have a molecular weight between about 200-10,000,000 daltons. In one embodiment, a supplemental RNA stabilizing substance may have a molecular weight between about 200-1,000,000 daltons. In one embodiment, a supplemental RNA stabilizing substance may have a molecular weight between about 200-500,000 daltons. In one embodiment, a supplemental RNA stabilizing substance may have a molecular weight between about 200-200,000 daltons. In one embodiment, a supplemental RNA stabilizing substance may have a molecular weight between about 200-100,000 daltons. In one embodiment, a supplemental RNA stabilizing substance may have a molecular weight between about 200-50,000 daltons. In one embodiment, a supplemental RNA stabilizing substance may have a molecular weight between about 200-20,000 daltons. In one embodiment, a supplemental RNA stabilizing substance may have a molecular weight between about 200-10,000 daltons. In one embodiment, a supplemental RNA stabilizing substance may have a molecular weight between about 200-5,000 daltons. In one embodiment, a supplemental RNA stabilizing substance may have a molecular weight between about 200-2,000 daltons. In one embodiment, a supplemental RNA stabilizing substance may have a molecular weight between about 200-1,000 daltons. In one embodiment, a supplemental RNA stabilizing substance may have a molecular weight between about 200-900 daltons. In one embodiment, a supplemental RNA stabilizing substance may have a molecular weight between about 200-800 daltons. In one embodiment, a supplemental RNA stabilizing substance may have a molecular weight between about 200-700 daltons. In one embodiment, a supplemental RNA stabilizing substance may have a molecular weight between about 200-600 daltons. In one embodiment, a supplemental RNA stabilizing substance may have a molecular weight between about 200-500 daltons.

One embodiment of the present invention is the method whereby one or more supplemental RNA stabilizing substance may be combined, such as by mixing, with at least one or more RNA substance to produce a mixture comprising at least one or more supplemental RNA stabilizing substance and at least one or more RNA substance. As a non-limiting example one or more RNA substance may be mixed with one or more supplemental RNA stabilizing substance.

Another embodiment of the present invention is the method whereby one or more supplemental RNA stabilizing substance may be combined, such as by mixing, with at least one or more RNA substance to produce a mixture comprising at least one or more RNA substance and at least one or more supplemental RNA stabilizing substance at one or more of the RNA stabilizing substance concentrations within the stabilizing substance molar concentration range list or in the stabilizing substance molar concentration list or the stabilizing substance weight percent concentration list as described herein. These same methods may be used to combine one or more other substances, including, but not limited to one or more cellular uptake agents or one or more additional RNA stabilizing substances, with one or more RNA substances and one or more supplemental RNA stabilizing substances to produce a mixture comprising one or more RNA substance, one or more supplemental RNA stabilizing substance, and one or more cellular uptake agent or one or more additional RNA stabilizing substance.

One embodiment of the present invention is the method whereby one or more supplemental RNA stabilizing substance may be combined, such as by mixing, with at least one or more RNA substance to produce a composition comprising at least one or more RNA substance and at least one or more supplemental RNA stabilizing substance. Another embodiment of the present invention is the method whereby one or more supplemental RNA stabilizing substance may be combined, such as by mixing, with one or more RNA substance to produce a composition comprising at least one or more RNA substance and at least one or more supplemental RNA stabilizing substance at one or more of the RNA stabilizing substance concentrations within the stabilizing substance molar concentration range list or in the stabilizing substance molar concentration list or stabilizing substance weight percent concentration list as described herein. These same methods may be used to combine one or more other substances, including, but not limited to one or more cellular uptake agents or one or more additional RNA stabilizing substance, with one or more RNA substance and one or more supplemental RNA stabilizing substance to produce a composition comprising one or more RNA substance, one or more supplemental RNA stabilizing substance, and one or more cellular uptake agent or one or more additional RNA stabilizing substance.

In one embodiment a supplemental RNA stabilizing substance may be polymer.

In one embodiment a supplemental RNA stabilizing substance may comprise one or more anionic moiety at about physiologic pH. In one embodiment a supplemental RNA stabilizing substance may be anionic at about physiologic pH. In one embodiment a supplemental RNA stabilizing substance may have a neutral charge at about physiologic pH.

In one embodiment a supplemental RNA stabilizing substance may comprise one or more PIF as described herein.

In one embodiment, one or more supplemental RNA stabilizing substance may be at least part of a polymer comprised of at least one or more supplemental RNA stabilizing substances.

In one embodiment one or more composition comprising one or more RNA substance and one or more supplemental RNA stabilizing substance may comprise one or more water concentrations in the water concentration list or in the water concentration range list. In one embodiment one or more composition comprising one or more RNA substance, one or more supplemental RNA stabilizing substance, and one or more additional substance, such as one or more cellular uptake agent or one or more additional RNA stabilizing substance as non-limiting examples, may comprise one or more water concentrations in the water concentration list or in the water concentration range list. In one embodiment one or more composition comprising one or more RNA substance and one or more supplemental RNA stabilizing substance may have a dielectric constant of one or more dielectric constant in the dielectric constant list or in the dielectric constant range list. In one embodiment one or more composition comprising one or more RNA substance, one or more supplemental RNA stabilizing substance, and one or more additional substance, such as one or more cellular uptake agent or one or more additional RNA stabilizing substance as non-limiting examples, may have a dielectric constant of one or more dielectric constant in the dielectric constant list or in the dielectric constant range list.

Embodiments of the present invention may include compositions comprising one or more supplemental RNA stabilizing substance used in a composition comprising one or more RNA substance, one or more supplemental RNA stabilizing substance, and one or more of the following substances: water, cellular uptake agents, aprotic substances, low dielectric substances, cyclic phosphate or metaphosphate containing substances, non-carbohydrate organic osmolyte substances, tertiary sulfonium containing substances, quaternary ammonium containing substances, quaternary phosphonium containing substances, hydrotrope containing substances, surfactant containing substances, betaine containing substances, stabilizing monomers, or stabilizing polymers. Other embodiments may include one or more composition comprising one or more supplemental RNA stabilizing substance and one or more RNA substance and may also comprise one or more cellular uptake agent or one or more additional RNA stabilizing substances. Other embodiments may include compositions comprising one or more supplemental RNA stabilizing substance, one or more RNA substance, and one or more additional substances, such as: one or more cellular uptake agent, or one or more additional RNA stabilizing substance, or one or more buffering agent, or one or more chelating agent, or one or more inorganic salt, or water, as non-limiting examples.

Embodiments of the present invention may include one or more compositions comprising one or more RNA substance and one or more supplemental RNA stabilizing substance, wherein a composition is not lyophilized.

In one embodiment one or more composition comprising one or more RNA substance and one or more supplemental RNA stabilizing substance may not be lyophilized. In one embodiment one or more composition comprising one or more RNA substance, one or more supplemental RNA stabilizing substance, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more supplemental RNA stabilizing substance, wherein a composition may be lyophilized.

In one embodiment one or more composition comprising one or more RNA substance and one or more supplemental RNA stabilizing substance may be lyophilized. In one embodiment one or more composition comprising one or more RNA substance, one or more supplemental RNA stabilizing substance, and one or more additional substance, such as one or more cellular uptake agent or one or more additional RNA stabilizing substance as non-limiting examples, may be lyophilized.

In one embodiment a supplemental RNA stabilizing substance may not be a lipid. In one embodiment a supplemental RNA stabilizing substance may not be a cholesterol. In one embodiment a supplemental RNA stabilizing substance is not a saccharide, polysaccharide, or starch.

In one embodiment a supplemental RNA stabilizing substance may not be a detergent. In one embodiment a supplemental RNA stabilizing substance may not be a peptide or polypeptide. In one embodiment a supplemental RNA stabilizing substance may not be protein. In one embodiment a supplemental RNA stabilizing substance may not be a nucleic acid base. In one embodiment a supplemental RNA stabilizing substance may not be a nucleic acid.

Embodiments of the present invention may include one or more compositions comprising one or more supplemental RNA stabilizing substance and one or more RNA substance described herein, wherein one or more composition described herein may have a melting of one or more melting point in the stabilizing composition melting point list.

In one embodiment one or more composition comprising one or more RNA substance and one or more supplemental RNA stabilizing substance may have a melting point of one or more melting point in the stabilizing composition melting point list. In one embodiment one or more composition comprising one or more RNA substance, one or more supplemental RNA stabilizing substance, and one or more additional substance, such as one or more cellular uptake agent or one or more additional RNA stabilizing substance as a non-limiting example, may have a melting point of one or more melting point in the stabilizing composition melting point list.

### RNA Stabilizing Substances:

The inventors have discovered that RNA stabilizing substances may stabilize RNA substances.

RNA stabilizing substances include one or more substances from one or more of the following categories: aprotic substances, low dielectric substances, cyclic phosphate or metaphosphate containing substances, non-carbohydrate organic osmolyte substances, tertiary sulfonium containing substances, quaternary ammonium containing substances, quaternary phosphonium containing substances, hydrotrope containing substances, surfactant containing substances, betaine containing substances, stabilizing monomers, stabilizing polymers, and supplemental RNA stabilizing substances.

The inventors have also discovered that the stability of RNA substances may be enhanced in an RNA storage environment comprising an RNA stabilizing substance. The inventors have also discovered that the stability of RNA substances may be enhanced in an RNA storage environment comprising an RNA stabilizing substance and one or more additional RNA stabilizing substance, such as an aprotic substance.

The inventors have also discovered that the stability of RNA substances may be enhanced in compositions comprising an RNA substance and an RNA stabilizing substance. The inventors have also discovered that the stability of RNA substances may be enhanced in compositions comprising an RNA substance, an RNA stabilizing substance, and one or more additional RNA stabilizing substance, such as an aprotic substance.

One embodiment of the present invention may include a composition comprised of one or more RNA substance and one or more RNA stabilizing substance. Another embodiment of the present invention may include a composition comprised of one or more RNA substance, one or more RNA stabilizing substance, and one or more additional RNA stabilizing substance, such as an aprotic substance.

One embodiment of the present invention may include a combination or mixture comprising one or more RNA substance and one or more RNA stabilizing substance. Another embodiment of the present invention may include a combination or mixture comprising one or more RNA substance, one or more RNA stabilizing substance, and one or more additional RNA stabilizing substance, such as an aprotic substance.

Embodiments of the present invention that comprise one or more RNA substance and one or more RNA stabilizing substance may include combining, such as by mixing, one or more RNA substance with one or more substance that comprises at least one or more RNA stabilizing substance.

Embodiments of the present invention that comprise one or more RNA substance, one or more RNA stabilizing substance, and one or more additional RNA stabilizing substance, such as an aprotic substance, may include combining, such as by mixing, one or more RNA substance with one or more substance that comprises at least one or more RNA stabilizing substance and one or more substance that comprises at least one or more RNA stabilizing substance, such as an aprotic substance.

An embodiment of the present invention may include compositions of materials that comprise one or more RNA substance and at least one or more RNA stabilizing substance. The storage environment that improves the stability of RNA substances may comprise at least one or more vapor, liquid, powder, or solid RNA stabilizing substance.

An embodiment of the present invention may include compositions of materials that comprise one or more RNA substance, one or more RNA stabilizing substance, and one or more additional RNA stabilizing substance, such as an aprotic substance. The storage environment that improves the stability of RNA substances may comprise at least one or more vapor, liquid, powder, or solid RNA stabilizing substance and may comprise at least one or more vapor, liquid, powder, or solid RNA stabilizing substance, such as an aprotic substance.

An embodiment of the present invention may include combinations of materials that comprise one or more RNA substance and at least one or more RNA stabilizing substance. The storage environment that improves the stability of RNA substances may comprise at least one or more vapor, liquid, powder, or solid RNA stabilizing substance.

An embodiment of the present invention may include combinations of materials that comprise one or more RNA substance, one or more RNA stabilizing substance, and one or more additional RNA stabilizing substance, such as an aprotic substance. The storage environment that improves the stability of RNA substances may comprise at least one or more vapor, liquid, powder, or solid RNA stabilizing substance and may comprise at least one or more vapor, liquid, powder, or solid RNA stabilizing substance, such as an aprotic substance.

In one embodiment of the present invention a composition comprised of one or more RNA substance and one or more RNA stabilizing substance, produces a mixture with at least one or more RNA substance and at least one or more RNA stabilizing substance. In one embodiment of the present invention a composition comprised of one or more RNA substance, one or more RNA stabilizing substance, and one or more additional RNA stabilizing substance, such as an aprotic substance, produces a mixture with at least one or more RNA substance, at least one or more RNA stabilizing substance, and at least one or more additional RNA stabilizing substance, such as an aprotic substance.

In one embodiment of the present invention a combination comprising one or more RNA substance and one or more RNA stabilizing substance, produces a mixture with at least one or more RNA substance and at least one or more RNA stabilizing substance. In one embodiment of the present invention a combination comprising one or more RNA substance, one or more RNA stabilizing substance, and one or more additional RNA stabilizing substance, such as an aprotic substance, produces a mixture with at least one or more RNA substance, at least one or more RNA stabilizing substance, and at least one or more additional RNA stabilizing substance, such as an aprotic substance.

In one embodiment of the present invention a composition with improved RNA stability may comprise one or more RNA substance and one or more RNA stabilizing substance. In one embodiment of the present invention a composition with improved RNA stability may comprise one or more RNA substance, one or more RNA stabilizing substance, and one or more additional RNA stabilizing substance, such as an aprotic substance.

In one embodiment of the present invention a composition with improved RNA stability may comprise a mixture of one or more RNA substance and one or more RNA stabilizing substance. In one embodiment of the present invention a composition with improved RNA stability may comprise a mixture of one or more RNA substance, one or more RNA stabilizing substance, and one or more additional RNA stabilizing substance, such as an aprotic substance.

In one embodiment of the present invention, each component included in a composition comprising one or more RNA substance and one or more RNA stabilizing substance, may be stored separately, such as in a kit, or such as individual substances or as mixtures of one or more substance, and then combined later to produce a composition comprising one or more RNA substance and one or more RNA stabilizing substance.

In one embodiment of the present invention, each component included in a composition comprising one or more RNA substance, one or more RNA stabilizing substance, and one or more additional RNA stabilizing substance, such as an aprotic substance, may be stored separately, such as in a kit, or such as individually or as mixtures of one or more substance, and then combined later to produce a composition comprising one or more RNA substance, one or more RNA stabilizing substance, and one or more additional RNA stabilizing substance, such as an aprotic substance.

In one embodiment of the present invention a composition comprising one or more RNA substance and one or more RNA stabilizing substance may be at least partially biocompatible.

In one embodiment of the present invention a composition comprising one or more RNA substance, one or more RNA stabilizing substance, and one or more additional RNA stabilizing substance, such as an aprotic substance, may be at least partially biocompatible.

In one embodiment of the present invention a combination comprised of one or more RNA substance and one or more RNA stabilizing substance may be at least partially biocompatible.

In one embodiment of the present invention a combination comprised of one or more RNA substance, one or more RNA stabilizing substance, and one or more additional RNA stabilizing substance, such as an aprotic substance, may be at least partially biocompatible.

Embodiments of the present invention comprising at least one or more RNA stabilizing substances may include one or more forms of the substance. These forms may include, but are not limited to, one more counterions, ionic forms, conjugate bases, conjugate acids, protonated or deprotonated forms, hydrated or dehydrated forms, isomers, structural isomers, stereo isomers, chiral forms, salts, or combinations thereof.

The inventors have discovered that the following categories of substances, described in detail elsewhere herein, may be RNA stabilizing substances: aprotic substances, low dielectric substances, cyclic phosphate or metaphosphate containing substances, non-carbohydrate organic osmolyte substances, tertiary sulfonium containing substances, quaternary ammonium containing substances, quaternary phosphonium containing substances, hydrotrope containing substances, surfactant containing substances, betaine containing substances, stabilizing monomers, stabilizing polymers, or supplemental RNA stabilizing substances.

The inventors have discovered that mixtures comprising at least one or more RNA substance and at least one or more RNA stabilizing substance may improve RNA stability. The inventors have also discovered that RNA stability may be improved with mixtures comprising at least one or more RNA substance and two or more RNA stabilizing substances. The inventors have also discovered that RNA stability may be improved with mixtures comprising at least one or more RNA substance and three or more RNA stabilizing substances. The inventors have also discovered that RNA stability may be improved with mixtures comprising at least one or more RNA substance and four or more RNA stabilizing substances. The inventors have also discovered that RNA stability may be improved with mixtures comprising at least one or more RNA substance and five or more RNA stabilizing substances.

Non-limiting examples of embodiments may include compositions comprising one or more RNA stabilizing substance and one or more RNA substance wherein a composition may comprise one or more of the following compositions:
1. DMSO, DMSP, Ectoine and at least one RNA substance.
2. HMP, choline, TMG and at least one RNA substance.
3. DMSO, NDSB-195, and at least one RNA substance.
4. Choline, carnitine, nicotinic acid and at least one RNA substance.
5. HMP, PTMAEMA and at least one RNA substance.
6. PTMAEMA, HMP TMG, and at least one RNA substance.
7. PDEAEMA, quinolinic acid, carnitine and at least one RNA substance.
8. Acetylcholine, benzoate, TMG, and at least one RNA substance.
9. Acetylcholine, DMSO, NDSB-195 and at least one RNA substance.
10. PTMAEMA, PMPC, ectoine, stachydrine and at least one RNA substance.
11. HMP, PMPC, choline, TMG, benzoate, and at least one RNA substance.
12. Benzoate, HMP, pipecolic acid betaine, and at least one RNA substance.
13. DMSO, NDSB-211, carnitine and at least one RNA substance.
14. Poly(carboxybetaine methacrylate-ethyl ester), alpha-GPC, HMP, and at least one RNA substance.
15. DMSO, benzoate, DMSP, poly(carboxybetaine methacrylate), and at least one RNA substance.
16. Valine betaine, benzoate, proline, choline, DMSP, and at least one RNA substance.
17. Dimethyl sulfone, acetylcholine, benzoate, proline betaine, and at least one RNA substance.
18. PDADMAC, PEG, glycerol phosphate, pipecolic acid betaine and at least one RNA substance.
19. Benzoate, TMG, trimethyloctylammonium, TMP and at least one RNA substance.
20. Taurine, 1-Methylnicotinamide, PVP, PTMAEMA, and at least one RNA substance.

Embodiments of the present invention may include combinations of substances comprising one or more RNA stabilizing substance wherein the concentration of one or more RNA stabilizing substance may be one or more concentration in the stabilizing substance molar concentration range list, or in the stabilizing substance molar concentration list, or in the stabilizing substance weight percent concentration list, or in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list, as described herein.

Other embodiments may include combinations of substances comprising one or more RNA stabilizing substance, one or more RNA substance, or one or more additional substance, such as one or more cellular uptake agent or one or more additional RNA stabilizing substance as non-limiting examples, wherein the concentration of one or more RNA stabilizing substance may be one or more concentration in the stabilizing substance molar concentration range list, or in the stabilizing substance molar concentration list, or in the stabilizing substance weight percent concentration list, or in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list, as described herein.

One embodiment of the present invention is the method whereby one or more RNA stabilizing substance may be combined, such as by mixing, with at least one or more RNA substance to produce a mixture comprising at least one or more RNA stabilizing substance and at least one or more RNA substance. As a non-limiting example one or more RNA substance may be mixed with one or more RNA stabilizing substance.

Another embodiment of the present invention is the method whereby one or more RNA stabilizing substance may be combined, such as by mixing, with at least one or more RNA substance to produce a mixture comprising at least one or more RNA substance and at least one or more RNA stabilizing substance at one or more of the RNA stabilizing substance concentrations within the stabilizing substance molar concentration range list, or in the stabilizing substance molar concentration list, or in the stabilizing substance weight percent concentration list, or in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. These same methods may be used to combine one or more RNA stabilizing substance with one or more RNA substance at one or more ratio in the stabilizing polymer C/P ratio list, or in the stabilizing polymer N/P ratio list, or in the stabilizing polymer C/P ratio range list, or in the stabilizing polymer N/P ratio range list. These same methods may be used to combine one or more other substances, including, but not limited to one or more cellular uptake agents or one or more additional RNA stabilizing substances, with one or more RNA substances and one or more RNA stabilizing substances to produce a mixture comprising one or more RNA substance, one or more RNA stabilizing substance, and one or more cellular uptake agent or one or more additional RNA stabilizing substance.

One embodiment of the present invention is the method whereby one or more RNA stabilizing substance may be combined, such as by mixing, with at least one or more RNA substance to produce a composition comprising at least one or more RNA substance and at least one or more RNA stabilizing substance. Another embodiment of the present invention is the method whereby one or more RNA stabilizing substance may be combined, such as by mixing, with one or more RNA substance to produce a composition comprising at least one or more RNA substance and at least one or more RNA stabilizing substance at one or more of the RNA stabilizing substance concentrations within the stabilizing substance molar concentration range list, or in the stabilizing substance molar concentration list, or in the stabilizing substance weight percent concentration list, or in the stabilizing polymer molar concentration range list, or in the stabilizing polymer molar concentration list, or in the stabilizing polymer milligram concentration range list, or in the stabilizing polymer milligram concentration list, or in the stabilizing polymer weight percent concentration range list, or in the stabilizing polymer weight percent concentration range list. These same methods may be used to combine one or more RNA stabilizing substance with one or more RNA substance at one or more ratio in the stabilizing polymer C/P ratio list, or in the stabilizing polymer N/P ratio list, or in the stabilizing polymer C/P ratio range list, or in the stabilizing polymer N/P ratio range list. These same methods may be used to combine one or more other substances, including, but not limited to one or more cellular uptake agents or one or more additional RNA stabilizing substance, with one or more RNA substance and one or more RNA stabilizing substance to produce a composition comprising one or more RNA substance, one or more RNA stabilizing substance, and one or more cellular uptake agent or one or more additional RNA stabilizing substance.

One embodiment of the present invention is the method whereby one or more RNA stabilizing substance may be combined, such as by mixing, with one or more RNA substance to produce a composition comprising one or more RNA stabilizing substance and one or more RNA substance, wherein a composition may not be lyophilized. As a non-limiting example one or more RNA substance may be mixed with one or more RNA stabilizing substance and a composition is not lyophilized. These same methods may be used to combine other substances including, but not limited to, cellular uptake agents or additional RNA stabilizing substances as non-limiting examples, with one or more RNA substances and one or more RNA stabilizing substances to produce a composition comprising one or more RNA substance, one or more RNA stabilizing substance, and one or more cellular uptake agent, wherein a composition may not be lyophilized.

One embodiment of the present invention is the method whereby one or more RNA stabilizing substance may be combined, such as by mixing, with one or more RNA substance to produce a composition comprising one or more RNA stabilizing substance and one or more RNA substance, wherein a composition may be lyophilized. As a non-limiting example one or more RNA substance may be mixed with one or more RNA stabilizing substance and a composition may be lyophilized. These same methods may be used to combine other substances including, but not limited to, cellular uptake agents or additional RNA stabilizing substances as non-limiting examples, with one or more RNA substances and one or more RNA stabilizing substances to produce a composition comprising one or more RNA substance, one or more RNA stabilizing substance, and one or more cellular uptake agent, wherein a composition may be lyophilized.

Embodiments of the present invention may be compositions that comprise at least one RNA stabilizing substance, at least one RNA substance, and water. These embodiments comprising water may be one or more composition as described herein that may also comprise water.

Embodiments of the present invention may comprise one or more composition described herein and one or more substances that are not RNA stabilizing substances or RNA substances of which water may be one substance that is not an RNA stabilizing substance or an RNA substance.

In one embodiment one or more composition comprising one or more RNA substance and one or more RNA stabilizing substance may comprise one or more water concentrations in the water concentration list or in the water concentration range list. In one embodiment one or more composition comprising one or more RNA substance, one or more RNA stabilizing substance, and one or more additional substance, such as one or more cellular uptake agent as a non-limiting example, may comprise one or more water concentrations in the water concentration list or in the water concentration range list. In one embodiment one or more composition comprising one or more RNA substance and one or more RNA stabilizing substance may have a dielectric constant of one or more dielectric constant in the dielectric constant list or in the dielectric constant range list. In one embodiment one or more composition comprising one or more RNA substance, one or more RNA stabilizing substance, and one or more additional substance, such as one or more cellular uptake agent as a non-limiting example, may have a dielectric constant of one or more dielectric constant in the dielectric constant list or in the dielectric constant range list.

In one embodiment one or more composition comprising one or more RNA substance and one or more RNA stabilizing substance may comprise a stabilizing complex, wherein a stabilizing complex may have at least one dimension with a mean perimeter of one or more dimension in the stabilizing complex mean perimeter dimension range list or stabilizing complex mean perimeter dimension list. In one embodiment one or more composition comprising one or more RNA substance, one or more RNA stabilizing substance, and one or more additional substance, such as one or more cellular uptake agent as a non-limiting example, may comprise a stabilizing complex, wherein a stabilizing complex may have at least one dimension with a mean perimeter of one or more dimension in the stabilizing complex mean perimeter dimension range list or stabilizing complex mean perimeter dimension list.

Embodiments of the present invention may include compositions comprising one or more RNA stabilizing substance used in a composition comprising one or more RNA substance, one or more RNA stabilizing substance, and one or more of the following substances: water, cellular uptake agents, aprotic substances, low dielectric substances, cyclic phosphate or metaphosphate containing substances, non-carbohydrate organic osmolyte substances, tertiary sulfonium containing substances, quaternary ammonium containing substances, quaternary phosphonium containing substances, hydrotrope containing substances, surfactant containing substances, betaine containing substances, stabilizing monomers, stabilizing polymers, or supplemental RNA stabilizing substances. Other embodiments may include one or more composition comprising one or more RNA stabilizing substance and one or more RNA substance and may also comprise one or more cellular uptake agent or one or more additional RNA stabilizing substances. Other embodiments may include compositions comprising one or more RNA stabilizing substance, one or more RNA substance, and one or more additional substances, such as: one or more cellular uptake agent, or one or more additional RNA stabilizing substance, or one or more buffering agent, or one or more chelating agent, or one or more inorganic salt, or water, as non-limiting examples.

Embodiments of the present invention may include one or more compositions comprising one or more RNA substance and one or more RNA stabilizing substance, wherein a composition is not lyophilized.

In one embodiment one or more composition comprising one or more RNA substance and one or more RNA stabilizing substance may not be lyophilized. In one embodiment one or more composition comprising one or more RNA substance, one or more RNA stabilizing substance, and one or more additional substance, such as one or more cellular uptake agent or one or more additional RNA stabilizing substance as non-limiting examples, may not be lyophilized.

Embodiments of the present invention may include one or more compositions comprising one or more RNA substance and one or more RNA stabilizing substance, wherein a composition may be lyophilized.

In one embodiment one or more composition comprising one or more RNA substance and one or more RNA stabilizing substance may be lyophilized. In one embodiment one or more composition comprising one or more RNA substance, one or more RNA stabilizing substance, and one or more additional substance, such as one or more cellular uptake agent or one or more additional RNA stabilizing substance as non-limiting examples, may be lyophilized.

Embodiments of compositions comprising one or more RNA stabilizing substance and one or more RNA substance may have different melting points. For conciseness, the following list of composition melting points is herein referred to as the stabilizing composition melting point list.

Embodiments of the present invention may include one or more compositions comprising one or more RNA substance and one or more RNA stabilizing substance, such as a stabilizing monomer or stabilizing polymer as non-limiting examples, wherein the composition may have a melting point greater than about 0° C. at standard atmospheric pressure. In one embodiment one or more composition comprising one or more RNA substance and one or more RNA stabilizing substance may have a melting point greater than about 2° C. at standard atmospheric pressure. In one embodiment one or more composition comprising one or more RNA substance and one or more RNA stabilizing substance may have a melting point greater than about 4° C. at standard atmospheric pressure. In one embodiment one or more composition comprising one or more RNA substance and one or more RNA stabilizing substance may have a melting point greater than about 6° C. at standard atmospheric pressure. In one embodiment one or more composition comprising one or more RNA substance and one or more RNA stabilizing substance may have a melting point greater than about 8° C. at standard atmospheric pressure. In one embodiment one or more composition comprising one or more RNA substance and one or more RNA stabilizing substance may have a melting point greater than about 10° C. at standard atmospheric pressure. In one embodiment one or more composition comprising one or more RNA substance and one or more RNA stabilizing substance may have a melting point greater than about 15° C. at standard atmospheric pressure. In one embodiment one or more composition comprising one or more RNA substance and one or more RNA stabilizing substance may have a melting point greater than about 20° C. at standard atmospheric pressure. In one embodiment one or more composition comprising one or more RNA substance and one or more RNA stabilizing substance may have a melting point greater than about 25° C. at standard atmospheric pressure. In one embodiment one or more composition comprising one or more RNA substance and one or more RNA stabilizing substance may have a melting point greater than about 30° C. at standard atmospheric pressure. In one embodiment one or more composition comprising one or more RNA substance and one or more RNA stabilizing substance may have a melting point greater than about 35° C. at standard atmospheric pressure. In one embodiment one or more composition comprising one or more RNA substance and one or more RNA stabilizing substance may have a melting point greater than about 40° C. at standard atmospheric pressure. In one embodiment one or more composition comprising one or more RNA substance and one or more RNA stabilizing substance may have a melting point greater than about 45° C. at standard atmospheric pressure. In one embodiment one or more composition comprising one or more RNA substance and one or more RNA stabilizing substance may have a melting point greater than about 50° C. at standard atmospheric pressure. In one embodiment one or more composition comprising one or more RNA substance and one or more RNA stabilizing substance may have a melting point greater than about 60° C. at standard atmospheric pressure. In one embodiment one or more composition comprising one or more RNA substance and one or more RNA stabilizing substance may have a melting point greater than about 70° C. at standard atmospheric pressure. In one embodiment one or more composition comprising one or more RNA substance and one or more RNA stabilizing substance may have a melting point greater than about 80° C. at standard atmospheric pressure. In one embodiment one or more composition comprising one or more RNA substance and one or more RNA stabilizing substance may have a melting point greater than about 90° C. at standard atmospheric pressure. In one embodiment one or more composition comprising one or more RNA substance and one or more RNA stabilizing substance may have a melting point greater than about 100° C. at standard atmospheric pressure. In one embodiment one or more composition comprising one or more RNA substance and one or more RNA stabilizing substance may have a melting point greater than about 120° C. at standard atmospheric pressure. In one embodiment one or more composition comprising one or more RNA substance and one or more RNA stabilizing substance may have a melting point greater than about 140° C. at standard atmospheric pressure. In one embodiment one or more composition comprising one or more RNA substance and one or more RNA stabilizing substance may have a melting point greater than about 160° C. at standard atmospheric pressure. In one embodiment one or more composition comprising one or more RNA substance and one or more RNA stabilizing substance may have a melting point greater than about 180° C. at standard atmospheric pressure. In one embodiment one or more composition comprising one or more RNA substance and one or more RNA stabilizing substance may have a melting point greater than about 200° C. at standard atmospheric pressure.

Embodiments of the present invention may include one or more compositions comprising one or more RNA stabilizing substance and one or more RNA substance described herein, wherein one or more composition described herein may have a melting of one or more melting point in the stabilizing composition melting point list.

In one embodiment one or more composition comprising one or more RNA substance and one or more RNA stabilizing substance may have a melting point of one or more melting point in the stabilizing composition melting point list. In one embodiment one or more composition comprising one or more RNA substance, one or more RNA stabilizing substance, and one or more additional substance, such as one or more cellular uptake agent as a non-limiting example, may have a melting point of one or more melting point in the stabilizing composition melting point list.

In one embodiment an RNA stabilizing substance may not comprise a lipid. In one embodiment an RNA stabilizing substance may not comprise a cholesterol. In one embodiment an RNA stabilizing substance may not comprise a saccharide, polysaccharide, or starch.

In one embodiment a RNA stabilizing substance may not comprise a detergent. In one embodiment a RNA stabilizing substance may not comprise a peptide or polypeptide. In one embodiment a RNA stabilizing substance may not be a polypeptide or protein. In one embodiment a RNA stabilizing substance may not comprise a nucleic acid base. In one embodiment a RNA stabilizing substance may not comprise a nucleic acid. In one embodiment a RNA stabilizing substance may not be a polynucleotide or poly nucleic acid.

### Cellular Uptake Agents:

In one embodiment of the present invention a combination that comprises one or more RNA stabilizing substance and one or more RNA substance also comprises one or more substance to promote the RNA's ability to enter cells (herein referred to as cellular uptake agents), example substances being, including but not limited to, lipids, polymers, polymeric materials, zwitterionic polymers, zwitterionic lipids, ionizable polymers, ionizable lipids, cationic polymers, cationic lipids, amino-lipids, cholesterols, cationic detergents, zwitterionic detergents, ionizable detergents, non-ionic detergents, detergents, polyethylenimine (PEI), polyplexes, polyamines, lipid nanoparticles, detergent micelles, micelles, liposomes, nanoliposomes, lipoparticles, nanolipoparticles, dendrimers, particles, nanoparticles, lipid membranes, lipid micelles, lipid bilayers, or membrane vesicles. Examples of cell entry may include, but are not limited to, fusion with the cellular membrane, endocytosis, pinocytosis, phagocytosis, passive diffusion, active diffusion, osmotic diffusion, facilitated diffusion, diffusion, hole formation, direct microinjection, electroporation, ultrasound, energy induced, electricity induced, electric field induced, or similar mechanisms to deliver the RNA substance to, including but not limited to, a cell, eukaryotic cell, prokaryotic cell, plant cell, fungal cell, plant, bacteria, fungus, insect, organ, tissue, animal, or vertebrate animal, including but not limited to a human, by entering cells.

As used herein, cellular uptake agents means substances that promote RNA's ability to enter cells, such as eukaryotic cells, prokaryotic cells, fungal cells, mammalian cells, animal cells, human cells, plant cells, bacterial cells, mycoplasma, or insect cells as non-limiting examples.

Cellular uptake agents are known art when used with RNA and may also be referred to as gene delivery agents, transfection agents, cellular delivery agents, intracellular delivery agents, or complexation agents. The present invention uses cellular uptake agents in the novel configuration of one or more cellular uptake agent with one or more RNA stabilizing substance and one or more RNA substance. At least one or more cellular uptake agent may be combined with at least one or more RNA stabilizing substance and at least one or more RNA substance either in advance and stored together or stored separately, such as in a two-chamber container, and combined close to the time of administration.

One embodiment of the present invention may include a composition comprised of one or more RNA substance, one or more RNA stabilizing substance, and one or more cellular uptake agent.

One embodiment of the present invention may include a combination or mixture comprising one or more RNA substance, one or more RNA stabilizing substance, and one or more cellular uptake agent.

Embodiments of the present invention that comprise one or more RNA substance, one or more RNA stabilizing substance, and one or more cellular uptake agent may include combining, such as by mixing, one or more RNA substance with one or more RNA stabilizing substance and one or more cellular uptake agent.

In one embodiment of the present invention a composition comprised of one or more RNA substance, one or more RNA stabilizing substance, and one or more cellular uptake agent produces a mixture with at least one or more RNA substance, at least one or more RNA stabilizing substance, and at least one or more cellular uptake agent.

In one embodiment of the present invention a combination comprising one or more RNA substance, one or more RNA stabilizing substance, and one or more cellular uptake agent produces a mixture with at least one or more RNA substance, at least one or more RNA stabilizing substance, and at least one or more cellular uptake agent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about −80° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about −80° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about −80° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about −80° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about −80° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about −60° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about −60° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about −60° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about −60° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about −60° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about −40° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about −40° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about −40° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about −40° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about −40° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about −30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about −30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about −30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about −30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about −30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about −20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about −20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about −20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about −20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about −20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about −10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about −10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about −10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about −10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about −10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about 0° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about 0° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about 0° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about 0° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about 0° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about 2° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about 2° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about 2° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about 2° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about 2° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about 4° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about 4° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about 4° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about 4° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about 4° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about 6° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about 6° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about 6° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about 6° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about 6° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about 8° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about 8° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about 8° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about 8° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about 8° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about 10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about 10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about 10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about 10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about 10° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about 20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about 20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about 20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about 20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about 20° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 50% of RNA molecules in an environment with temperatures exceeding a defined temperature of about 30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 40% of RNA molecules in an environment with temperatures exceeding a defined temperature of about 30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 30% of RNA molecules in an environment with temperatures exceeding a defined temperature of about 30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 20% of RNA molecules in an environment with temperatures exceeding a defined temperature of about 30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As a non-limiting example, a composition comprising at least one RNA substance and at least one RNA stabilizing substance may stabilize the RNA substance to degrade no more than about 10% of RNA molecules in an environment with temperatures exceeding a defined temperature of about 30° C. for at least one of about 1 hour, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples the exposure to temperatures of at least the defined temperature may be continuous or the exposure may be intermittent.

As used herein, chamber means a compartment capable of containing at least one of a solid, powder, liquid, aerosol, or gas that may be sealed and later allow at least some of its contents to be at least one of partially delivered, removed, emptied, dispensed, opened, accessed, or penetrated. As a non-limiting example, a chamber may be at least one of, including but not limited to, bottles, containers, vials, tubes, jars, syringes (including prefilled syringes), blisters, capsules, tablets, cartridges, inhalers, packets, pods, bags, boxes, or other packages that may hold a solid, powder, liquid, aerosol, or gas. As a non-limiting example, chambers may contain up to 100,000 kg of stabilized RNA composition, or may contain up to 10,000 kg of stabilized RNA composition, or may contain up to 1,000 kg of stabilized RNA composition, or may contain up to 100 kg of stabilized RNA composition or may contain up to 10 kg of stabilized RNA composition, or may contain up to 1 kg of stabilized RNA composition, or may contain up to 100 g of stabilized RNA composition, or may contain up to 10 g of stabilized RNA composition, or may contain up to 1 g of stabilized RNA composition, or may contain up to 100 mg of stabilized RNA composition, or may contain up to 10 mg of stabilized RNA composition, or may contain up to 1 mg of stabilized RNA composition, or may contain up to 100 μg of stabilized RNA composition, or may contain up to 10 μg of stabilized RNA composition.

In one embodiment of the present invention a chamber may comprise one or more RNA substance with one or more RNA stabilizing substance. In one embodiment the chamber may be a vial. In one embodiment the chamber may be a prefilled syringe. In one embodiment the chamber may be a capsule or tablet.

In one embodiment of the present invention a chamber may comprise one or more RNA substance with one or more RNA stabilizing substance and one or more cellular uptake agent. In one embodiment the chamber may be a vial. In one embodiment the chamber may be a prefilled syringe.

In one embodiment of the present invention a chamber may comprise one or more RNA substance with one or more RNA stabilizing substance, one or more cellular uptake agent and one or more buffering agent. In one embodiment the chamber may be a vial. In one embodiment the chamber may be a prefilled syringe.

In one embodiment of the present invention a chamber may comprise one or more RNA substance with one or more RNA stabilizing substance, one or more cellular uptake agent, one or more buffering agent and one or more inorganic salt. In one embodiment the chamber may be a vial. In one embodiment the chamber may be a prefilled syringe.

In one embodiment of the present invention a chamber may comprise one or more RNA substance with one or more RNA stabilizing substance, one or more cellular uptake agent, one or more buffering agent, one or more inorganic salt, and one or more chelating agent. In one embodiment the chamber may be a vial. In one embodiment the chamber may be a prefilled syringe.

In one embodiment of the present invention, each component included in a composition comprising one or more RNA substance, one or more RNA stabilizing substance, and one or more cellular uptake agent, may be stored separately, such as in a kit, or such as individually or as mixtures of one or more substance, and then combined later to produce a composition comprising one or more RNA substance, one or more RNA stabilizing substance, and one or more cellular uptake agent.

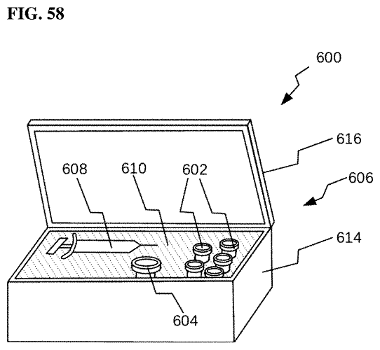

An example of such a kit 600 is shown in FIG. 58. As shown, the kit 600 includes one or more component vials 602 and one or more mixing/dispensing vials 604 contained in a package 606, such as a hinged box container. Each of the component vials 602 may contain one or more components of the composition as described herein. For example, each component may be provided in a separate vial 602 or certain compatible components may be combined in a single vial 602 with other components provided individually or in combination in other vials 602.

The components can then be mixed in mixing/dispensing vial 604, for example, immediately prior to use, in order to minimize RNA degradation. In this regard, the components from the vials 602 may be transferred into the vial 604, e.g., poured or injected into the vial 604 using a syringe 608, and then shaken or otherwise mixed. The components in the vials 602 may be premeasured and may provide a single unit or dose of the composition or multiple units/doses. Alternatively, the components from the vials 602 may be measured and combined by skilled workers. Optionally, e.g., in the case of vaccines, one or more syringes 608 may be provided in the kit 600 for drawing the composition from the vial 604 and administering to subjects. The vials 602 and 604 and syringes 608 may be secured by packing material 610 such as a foam material.

As described herein, many compositions, components, or combinations of components in accordance with the present invention can be stored without requiring extremely low temperatures. In cases where cold storage is required, the kit 600 can be transported in a cold storage unit or cold storage vehicles. The packaging 606 may be formed from materials suitable to withstand such cold storage such as various plastics or metals. In such cases, the vials 602, 604 and syringes 608 (if provided in the kit 600) may be formed from materials selected to withstand cold storage.

Although the kit is shown as including vials 602, 604 and syringes 608 for purposes of illustration, it will be appreciated that the components may be provided in other forms, e.g., non-liquid forms, and the composition may be provided for purposes other than vaccination. Accordingly, while a kit including some or all of the components of a composition in accordance with the present invention is useful and convenient, the nature of the kit can vary from the kit shown.

As a non-limiting example, vials 602 may comprise a concentrated composition comprising at least one RNA substance and at least one RNA stabilizing substance that after being mixed with at least one diluent is suitable for use, such as suitable for injection. In this embodiment the diluent may or may not be part of kit 600.

In another embodiment packaging 606 may comprise vials 602 that may be ready for use and packaging 606 provides a package for uses comprising at least one of as storage and transport and maintaining a desired environment for vials 602. Packaging 606 may comprise a cooling pack (not shown) that at least partly offsets thermal energy transferred from the storage and transport environment to the inside of package 606 where at least some of vials 602 are desired to experience a maximum target temperature.

In one embodiment kit 600 may comprise a cooling substance (not shown) that maintains the temperature of at least one of vials 602 at no more than a maximum target temperature. As a non-limiting example, the maximum target temperature may be about 4° C. or less. As a non-limiting example, the maximum target temperature may be about 20° C. or less. As a non-limiting example, a cooling substance that maintains the maximum target temperature or less may undergo a phase change to maintain the maximum target temperature. Non-limiting examples of cooling substances that may be used to maintain the maximum target temperature are solid phase water that may change to liquid phase water, solid phase carbon dioxide that may change phase to vapor phase carbon dioxide, or liquid phase nitrogen that may change from liquid phase to vapor phase nitrogen. Other non-limiting examples of cooling substances that may be used to maintain the maximum target temperature are refrigerants circulated through heat exchangers used in compression refrigeration (for example, haloalkane refrigerants or hydrocarbons), adsorption refrigeration, or absorption refrigeration. As a non-limiting example, the maximum target temperature or less may be maintained using at least one substance that does not undergo phase change. As non-limiting examples of cooling substances that may be used to maintain the maximum target temperature or less that do not undergo phase changes are thermoelectric coolers of which Peltier devices that may be incorporated into one or more walls of package 606 are non-limiting examples.

As non-limiting examples the cooling substance used to maintain the maximum target temperature or less may be cooling packs comprising a package containing one or more substances that maintain the target temperature or less. A non-limiting example of a cooling pack is a plastic package containing water that may be cooled to be at or less than the target temperature including being cooled to produce at least some water ice in the package. As non-limiting examples the cooling substance may comprise water and at least one substance that when mixed with water increases the viscosity. As non-limiting examples, the substance mixed with water to increase viscosity may be hydroxyethyl cellulose, sodium polyacrylate, or vinyl-coated silica gel. As non-limiting examples, the cooling substance used to maintain the maximum target temperature or less may comprise a package containing water and a viscosity increasing material and the viscosity increasing material may comprise at least one of hydroxyethyl cellulose, sodium polyacrylate, or vinyl-coated silica gel. As non-limiting examples, the cooling substance used to maintain the maximum target temperature or less (as a non-limiting example, comprising a package, water, and a viscosity increasing substance) the package may be placed in an environment that is colder than the maximum target temperature before use to maintain the target temperature. As a non-limiting example, the substance may comprise a flexible plastic package, water, and a viscosity increasing substance and the package with water and a viscosity increasing material may be placed in a freezer to cool the package before it is used to maintain the target temperature or less. As non-limiting examples, frozen gel-packs may be used to maintain the maximum target temperature or less.

As a non-limiting example, kit 600 may comprise at least one substance comprised of two or more materials that when mixed cause an endothermic chemical reaction that leads to absorbing energy from the surroundings to maintain the temperature of at least one vial 602 at a maximum target temperature or less. As a non-limiting example, the substance maintaining the temperature at the maximum target temperature or less may comprise a flexible plastic package containing at least one endothermic producing compound and also contain a sealed package holding water such that the inner bag of water can be broken by a user to cause the water to mix with at least one endothermic producing compound. As a non-limiting example, a user may break the inner bag of water by squeezing the package, allowing the water to mix with at least one endothermic producing compound to produce endothermic reactions, followed by the user placing the package in kit 600 to maintain the temperature of at least on vial 602 at the maximum target temperature. As non-limiting examples, the endothermic producing compound may be at least one of ammonium nitrate, calcium ammonium nitrate, or urea. As a non-limiting example, kit 600 may comprise a substance that maintains the temperature of at least one vial 602 at a maximum target temperature or less in which the temperature maintaining substance comprises water and an endothermic producing compound in which the water and endothermic producing compound may be separated until use and be available for mixing by having the water and endothermic producing compound in a package that comprises an inner package that holds water separated from endothermic producing compounds until the materials are mixed.

As a non-limiting example, one embodiment of the present invention is a kit comprising at least one RNA substance and at least one RNA stabilizing substance in which such kit has a package 606 with inside volume that allows maintaining a maximum target temperature or less by placing at least one cooling pack. Non-limiting examples of cooling packs include endothermic cooling packs or gel-packs that have been cooled such as by having been in a freezer or been in an environment with a cold substance of which non-limiting examples include dry ice or water ice or water ice mixed with brine.

A non-limiting example embodiment of kit 600 comprises component vials 602 that may contain one or more components of the composition as described herein in which at least one component is an RNA substance and at least one component is an RNA stabilizing substance (which may be an aprotic substance) and a substance that maintains the maximum target temperature of at least one of the vials 602 to about 4° C. or less for at least one hour. In a non-limiting embodiment the kit may maintain the temperature of at least one vial at about 20° C. or less for at least 24 hours. As another non-limiting embodiment, kit 600 may maintain the temperature of at least one vial at about 20° C. or less for at least 60 hours.

Non-limiting embodiments of kits comprise at least one RNA substance, at least one RNA stabilizing substance, a package, and at least one of a temperature recording device and a location tracking device. A non-limiting example of temperature recording devices may comprise a temperature sensitive substance that irreversibly changes to indicate when a specified temperature has been exceeded, such as by causing a chemical of physical change visible to a user. As non-limiting example of temperature recording devices are devices comprising temperature measuring and logging devices that comprise one or more electronic component that may be thermocouples, thermistors, or other temperature sensing element. Non-limiting examples of temperature recording devices are devices with at least one electronic communication component comprising a visual display or a radio-frequency transmitter and may comprise wi-fi or cellular communication capability. Non-limiting examples of location tracking devices may comprise GPS or inertial location devices with at least one electronic communication component comprising a visual display or a radio-frequency transmitter and may comprise wi-fi or cellular communication capability.

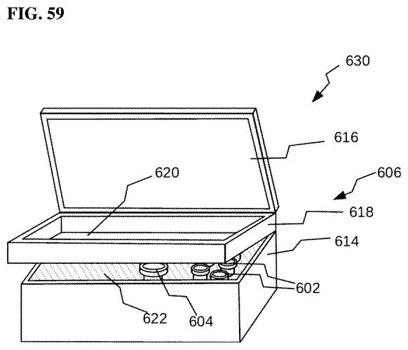

FIG. 59 illustrates a non-limiting example embodiment of the present invention as kit 630 that comprises component vials 602 that may contain one or more components of the composition as described herein in which at least one component is an RNA substance and at least one component is an RNA stabilizing substance in package 606 that may have cooling substance compartment 618 that has cooling substance chamber 620 that may contain a cooling substance with cooling substance chamber 620 at least partly separated from component compartment 622 containing at least one of vials 602. Package 606 comprises container box base 614, container box cover 616, and cooling substance compartment 618. Cooling substance compartment 618 may form at least part of the cover of container box base 614 holding one or more vials 602. Container box cover 616 may form at least part of a cover for cooling substance compartment 618. Cooling substance compartment 618 may have cooling substance chamber 620 into which may be placed a cooling substance of which a non-limiting example is a cooling pack. The non-limiting example kit illustrated in FIG. 59 has cooling substance chamber 620 separate from component compartment 622 containing vials 602, a configuration that allows replacing cooling substance without opening the compartment containing other components of the kit, such as vials 602, providing secure storage of those components when cooling substances are added or replenished. FIG. 59 illustrates separation of at least some vials 602 from cooling substances using cooling substance compartment 618. As a non-limiting alternative example, the separation of cooling substances from a compartment containing at least of vials 602 may be one or more packages such as bags or boxes used with cooling substances which, as non-limiting examples, may be packs containing ice, cooling gel packs, or endothermic cooling packs.

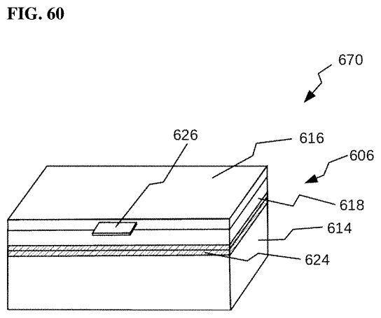

FIG. 60 illustrates that a kit comprising at least one RNA substance and at least one RNA stabilizing substance may comprise a security measure 624 that indicates whether the kit has been opened. As a non-limiting example, security measure 624 may be an adhesive sealing strip that changes appearance when removed or tampered with, such as by having at least one of a color or pattern change. Security measure 624 may also help fix one part of a kit comprising an RNA substance and an RNA stabilizing substance to another part of the kit. A non-limiting example is two parts of kit package 606 where security measure 624 may be a band with adhesive that helps fix container box base 614 to cooling substance compartment 618. As non-limiting examples, security measure 624 may comprise an adhesive strip that attaches in part to container box base 614 and in part to cooling substance compartment 618. As another non-limiting example, security measure 624 may be used with kits to help secure a container box cover to a container base or cooling substance compartment. In other embodiments security measure 624 may comprise a mechanical lock.

FIG. 60 illustrates that a kit comprising at least one RNA substance and at least one RNA stabilizing substance may comprise a latch 626 that reversibly fixes at least one component of the kit to another component. As non-limiting examples, latch 626 may reversibly fix container box cover 616 to cooling substance compartment 618 as shown in FIG. 60. Other non-limiting latching configurations may also be used, such as non-limiting examples where latch 626 reversibly fixes container box cover 616 to container box base 614 and where a latch reversibly fixes cooling substance compartment 618 to container box base 614. More than one latch may be used to reversibly fix components of the kit together. Non-limiting examples of latches are over-center toggle latches, magnetic latches, or hasps that may be locked or fixed with tamper-evident indicators such as wires.

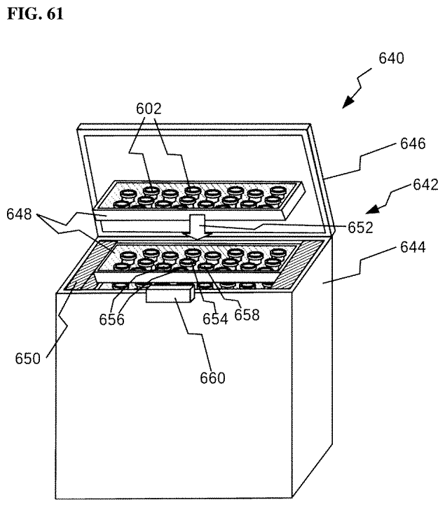

FIG. 61 illustrates container 640 as a non-limiting example of a shipping and storage container that may be used, for example, to package chambers with compositions comprising at least one RNA substance and at least one RNA stabilizing substance. As a non-limiting example, the chambers may comprise vials or pre-filled syringes that may comprise an RNA substance, an RNA stabilizing substance and a cellular uptake agent. As non-limiting examples, FIG. 61 illustrates the chambers as vials 602 and for consistency in this description chambers are referred to as vials 602. Container 640 may comprise component vials 602 that may contain one or more components of the composition as described herein in which at least one component is an RNA substance and at least one component is an RNA stabilizing substance in package 642 that may have component vials 602 in at least one carrier 648 which, as a non-limiting example, may be a tray. Non-limiting alternative embodiments may comprise vials 602 containing ready to use mixtures comprising at least one RNA substance and at least one RNA stabilizing substance or concentrated mixtures comprising at least one RNA substance and at least one RNA stabilizing substance that when diluted are suitable for use. As a non-limiting example, a diluent may be a substance comprising water or other biocompatible substance.

Package 642 comprises container box 644 holding one or more carriers 648. Package 642 may comprise at least one opening at least partly reversibly closable with at least one cover 646. Cover 646 and the associated opening may be located at or near the top of package 642 or cover 646 and the associated opening may be located on one or more sides or the bottom of package 642.

In one non-limiting embodiment at least one of container box 644 and cover 646 may comprise at least one material having thermal conductivity less than about 0.2 W/m ° K. In one non-limiting embodiment, at least one of container box 644 and cover 646 may comprise at least one material having thermal conductivity less than about 0.1 W/m ° K. In one non-limiting embodiment at least one of container box 644 and cover 646 may comprise at least one material having thermal conductivity less than about 0.05 W/m ° K. In one non-limiting embodiment at least part of at least one of container box 644 and cover 646 may have an overall heat transfer coefficient less than about 20 W/m²° K. In one non-limiting embodiment at least part of at least one of container box 644 and cover 646 may have an overall heat transfer coefficient less than about 10 W/m²° K. In one non-limiting embodiment at least part of at least one of container box 644 and cover 646 may have an overall heat transfer coefficient less than about 5 W/m²° K. In one non-limiting embodiment at least part of at least one of container box 644 and cover 646 may have an overall heat transfer coefficient less than about 2 W/m²° K. In one embodiment package 642 has at least one coolant chamber 650 that may contain a cooling substance with coolant chamber 650 at least partly separated from at least one of carriers 648 containing at least one of vials 602. As non-limiting examples, one or more coolant chamber 650 may be at least partly on or near one or more sides of container box 644 as illustrated in FIG. 61 or one or more cooling chamber 650 may be at least partly on or near the bottom of container box 644 or one or more cooling chamber 650 may be at least partly on or near the top of container box 644 one or more cooling chamber 650 may be at least partly part of or attached to cover 646. Package 642 may comprise container box 644, container box cover 646, and cooling chamber 650. More than one carrier 648 may be placed in container box 644. In a non-limiting example, carriers 648 may be placed to form a vertical stack of trays by placing trays one on top of the other, possibly with at least one separator (not shown) between carriers 648, as illustrated by arrow 652 indicating a vertical insertion of carriers 648 into container box 644.

Carriers 648 may be made from polymeric substances using known methods with example methods being injection molding, blow-molding, pressure forming, casting, machining, or vacuum forming. In one non-limiting embodiment carriers 648 may be formed with individual compartments (not shown) for vials 602. As a non-limiting example, such compartments may be sized to at least partly separate vials or to at least partly secure vials 602. Such at least partial separation or at least partial securing may reduce impact, vibrations, or contacting vials to reduce the possibility of breakage or other damage during shipping, transport, storage or other movement of vials 602.

Carriers 648 may be any size convenient for shipping and use. As non-limiting examples, carriers 648 may have a length or width of between about 10 cm and 100 cm and carriers may have a length or width between about 30 cm and 40 cm. As non-limiting examples, carriers 648 may have a length or width between about 10 cm and about 20 cm.

In one embodiment, carriers 648 may nest with each other or may nest with vials 602 to facilitate securing vials in the carriers. For example, the bottoms of carriers 648 may have recesses that mate with the tops of vials 602 such that when a carrier is removed the absence of the bottom recess in the removed carrier reveals a section of the vial beneath the carrier that may be grasped but when the carrier is in place the recess helps maintain the positions of vials 602 during shipping and storage. It will be appreciated that although carriers 648 are illustrated and described as holding vials 602 vertically that other orientations may also be employed, such as carriers being inserted vertically and holding vials 602 vertically or holding vials 602 in a horizontal orientation. As a non-limiting example, the compartments of carriers 648 may be configured to have interference fits with vials 602. As a non-limiting example of an interference fit, at least a portion of the openings of compartments may be smaller than a dimension of vials 602 so that vials 602 press into the openings to be removably secured in compartments of carriers 648. As a non-limiting example, trays may have compartments that hold vials 602 with the axis of the vials parallel to the plane of carrier 648 such that when carrier 648 is inserted into container box 644 with carriers 648 at least partially vertically oriented the vials 602 are positioned with the axis of the vials being at least partially vertically oriented. As a non-limiting example, when carriers 648 are oriented at least partially vertically the opposing face of the adjacent carrier may be shaped to allow a single carrier to be withdrawn from container box 644 without adjusting the positions of other carriers 648. Alternatively, carriers may be positioned horizontally with the opening associated with cover 646 positioned on the side of container box 644. It will be appreciated that chambers other than vials may be used to contain RNA substances and RNA stabilizing substances and other materials as described herein to have mixtures of RNA substances and RNA stabilizing substances and such other chambers may be substituted for at least some of vials 602. When chambers other than vials are present in carriers 648 the carriers may be configured to both provide access to chambers when carriers are removed and to help maintain positions of chambers during shipping and storage when carriers are in place, similar to as described above for vials.

As a non-limiting example, at least one carrier 648 may have vials arranged to efficiently pack vials 602 to save space compared to less efficient packing arrangements. A non-limiting example of efficiently packing vials 602 is a hexagonal cell surrounding a central vial. FIG. 61 illustrates a hexagonal cell with central vial 654 surrounded by six other approximately equally spaced vials with examples of surrounding vials being vials 656 and 658. In one non-limiting embodiment, at least one carrier 648 may have compartments into which vials 602 may be placed to efficiently pack them with one non-limiting example being at least one carrier 648 having compartments that may hold at least seven vials in an approximately hexagonal pattern with at least one vial surrounded by six other vials that are approximately equally spaced from the central vial and from each other with the spacing between centers being between about 5 percent and 50 percent greater than the diameter of vials 602.

In one non-limiting embodiment, package 642 may have a latch 660 that reversibly holds cover 646 in a closed position on box 644, with FIG. 61 illustrating as a non-limiting example latch 658 attached to box 644 and capable of reversibly securing cover 646 to close the opening of box 644.

As a non-limiting example, container 640 may comprise least one of a temperature recording device and a location tracking device as described earlier. As a non-limiting example, container 640 may comprise a security measure as described earlier.

In non-limiting embodiments of the present invention package 642 may be cooled using phase change substances of which non-limiting examples are the types described earlier. As non-limiting examples, vials 602 may comprise at least on RNA substance and at least RNA stabilizing substance and one or more substances other than dry ice may be used as the cooling substance in one or more compartment 650 to maintain a temperature substantially warmer than the −78° C. for which dry ice is used.

Non-limiting embodiments of the present invention may comprise RNA substances and RNA stabilizing substances and water ice as a cooling substance to simplify shipping and storage of RNA substances by not using dry ice. When dry ice is used as the cooling substance the dry ice sublimates to offset the energy entering by thermal conduction into the chamber containing RNA substances. The thermal energy transfer rate (heat transfer rate) may be approximated by the expression Q=U×A×ΔT where Q is the heat transfer rate, U is the overall heat transfer coefficient, and ΔT is the temperature difference between the inside of the package containing the RNA substance and the environment. The heat transfer rate is proportional to that temperature difference. ΔT=Tₑ−Tᵥ where Tₑ=the temperature of the outside environment, such as 20° C. and Tᵥ=the target temperature at which vials of RNA substance may be maintained. Dry ice may be used when low Tᵥ is required, such as −78° C., leading to ΔT=20−(−78), equal to 98° C. As a non-limiting example, using RNA stabilizing substances and Tᵥ=4° C. leads to ΔT=20-4, equal to 16° C. For this example ΔT with dry ice is over six times greater than when using RNA stabilizing substances. The result is that the thermal energy transfer rate is over six times greater when RNA stabilizing substances are not used and dry ice is required. The cooling capacity of dry ice comes from it sublimating at −78° C. at which 199 kJ/kg of energy is absorbed. The density of dry ice is about 1,562 kg/m³ so each liter of dry ice will absorb about 311 kJ. The cooling capacity of water ice comes from it melting at 0° C. at which 334 kJ/kg of energy is absorbed. Accounting for the density of water ice (917 kg/m³) each liter of water ice will absorb about 306 kJ. These results demonstrate that a shipping container designed to hold a specific volume of cooling substance will have about the same amount of cooling capacity for both dry ice and water ice. However, with the RNA stabilizing substance and using Tv=4° C. the rate of heat gain with water ice is about one-sixth that which occurs when using dry ice so the shipping container with water ice will maintain the target vial temperature much longer. Alternatively, a shipping package comprising an RNA substance and an RNA stabilizing substance may be smaller when cooled with water ice than a shipping package that uses dry ice and no RNA stabilizing substance.

The preferred thickness of insulating walls of package 642 is approximately equal to the thickness of cooling chamber 650. The time that a cooling substance can cool a package is

$$τ = \frac{C}{Q}$$

where C=cooling capacity of cooling substance (Joules), Q=thermal energy transfer rate (J/hr).

C=t_{c}AH

where t_{c}=thickness of the cooling substance, A=area of face of cooling material against, for example, a wall of the package. H=energy absorbing capacity per unit volume of cooling substance.

$$Q = \begin{matrix}kA Δ T \\ {t}_{i}\end{matrix}$$

where k=thermal conductivity of side wall, tᵢ=thickness of the insulated side wall, A=area of side wall (assumed to be the same as area of the cooling substance against that wall), ΔT=temperature difference between the cooling substance and the environment.

After substitution obtain

$$τ = \frac{{t}_{c} {Ht}_{i}}{k Δ T}$$

know that that the total thickness is the thickness of the insulation thickness plus the thickness of the cooling substance, therefore the thickness of the cooling substance is the difference between the total thickness and insulation thickness.

t_{c}=tₜ−tᵢ

After substitution obtain

$$τ = \frac{( {t}_{i} {t}_{t} - {t}_{i}^{2} ) H}{Δ T k}$$

Take derivative and set to 0 to find optimum and solve for optimum insulation thickness

$$\frac{d τ}{{dt}_{i}} = \frac{( {t}_{t} - 2 {t}_{i} ) H}{Δ Tk}$$

$${t}_{i} = \frac{1}{2} {t}_{t}$$

Optimum insulation thickness is about one-half of total thickness of the insulation and the cooling substance, i.e., the optimum insulation thickness is about the same thickness as the cooling substance thickness, and is independent of cooling substance, temperature difference, and insulation thermal conductivity.

In one embodiment of the present invention a composition comprising one or more RNA substance, one or more RNA stabilizing substance, and one or more cellular uptake agent may be at least partially biocompatible.

In one embodiment of the present invention a combination comprised of one or more RNA substance, one or more RNA stabilizing substance, and one or more cellular uptake agent, may be at least partially biocompatible.

In another embodiment at least one or more cellular uptake agent may be combined with at least one or more RNA stabilizing substance, as described herein, and at least one or more RNA substance either in advance and stored together or stored separately, such as in a two-chamber container, and combined close to the time of administration.

### Examples of Cellular Uptake Agents:

In one embodiment a cellular uptake agent may comprise, but is not limited to, at least one or more of the following: a lipid, polymer, polymeric material, zwitterionic polymer, zwitterionic lipid, ionizable polymer, ionizable lipid, cationic polymer, cationic lipid, amino-lipid, cholesterol, cationic detergent, zwitterionic detergent, ionizable detergent, non-ionic detergent, detergent, polyethylenimine (PEI), polyplexes, polyamines, lipid nanoparticles, detergent micelles, micelles, liposomes, nanoliposomes, lipoparticles, nanolipoparticles, dendrimers, particles, nanoparticles, lipid membrane, lipid micelle, lipid bilayer, or membrane vesicles, or derivatives, mixtures, or combinations thereof.

In another embodiment a cellular uptake agent may comprise, but is not limited to, at least one or more of the following surrounding an aqueous core or a hydrophobic core: a lipid, polymer, polymeric material, zwitterionic polymer, zwitterionic lipid, ionizable polymer, ionizable lipid, cationic polymer, cationic lipid, amino-lipid, cholesterol, cationic detergent, zwitterionic detergent, ionizable detergent, non-ionic detergent, detergent, polyethylenimine (PEI), polyplexes, polyamines, lipid nanoparticles, detergent micelles, micelles, liposomes, nanoliposomes, lipoparticles, nanolipoparticles, dendrimers, particles, nanoparticles, lipid membrane, lipid micelle, lipid bilayer, or membrane vesicles, or derivatives, mixtures, or combinations thereof.

As used herein, PEG is polyethylene glycol.

As used herein, a PEG lipid is a lipid modified with or conjugated to polyethylene glycol.

In another embodiment a cellular uptake agent may comprise, but is not limited to one or more the following: phospholipids, sterols, cholesterol, phospholipid-free lipid particle, non-cationic lipid, cholesterol-free lipid particle, noncyclic phosphate containing lipids, lipid conjugates, PEG-conjugated lipids, PEG-lipid conjugates, ATTA-lipid conjugates, cationic-polymer-lipid conjugates, PEG coupled to dialkyloxypropyls, PEG coupled to diacylglycerol, PEG coupled to phospholipids such as phosphatidylethanolamine, PEG conjugated to ceramides, or PEG conjugated to cholesterol, or derivatives, mixtures, or combinations thereof.

In some embodiments, a cellular uptake agent may comprise a polyethylene glycol-lipid, PEG or PEG-modified lipids (also known as PEGylated lipids), including but not limited to, at least one or more of the following: PEG-modified phosphatidylethanolamines, PEG-modified phosphatidic acids, PEG-modified ceramides, PEG-modified dialkylamines, PEG-modified diacylglycerols, or PEG-modified dialkylglycerols, or derivatives, mixtures, or combinations thereof.

In another embodiment a cellular uptake agent may comprise, including but not limited to, at least one or more of the following hydrophilic polymers substituted for or used in combination with PEG as described herein: polyvinylpyrrolidone, polymethyloxazoline, polyethyloxazoline, polyhydroxypropyl methacrylamide, polymethacrylamide or polydimethylacrylamide, polylactic acid, polyglycolic acid, or derivatized celluloses such as hydroxymethylcellulose or hydroxyethylcellulose, or derivatives, combinations, or mixtures thereof.

In another embodiment a cellular uptake agent may comprise, including but not limited to, at least one or more of the following: cholesterol, fecosterol, sitosterol, ergosterol, campesterol, stigmasterol, brassicasterol, tomatidine, tomatine, ursolic acid, alpha-tocopherol, corticosteroids, prednisolone, dexamethasone, prednisone, or hydrocortisone, or derivatives, combinations, or mixtures thereof.

In some embodiments, a cellular uptake agent may comprise a polymer, cationic polymer, cationic or polycationic compounds, or cationic polysaccharides, for example chitosan, polybrene, or polyethyleneimine (PEI), or derivatives, combinations, or mixtures thereof.

In some embodiments, a cellular uptake agent may comprise, including but not limited to, one or more of cationic peptides or proteins, cell penetrating peptides, basic polypeptides, basic amino acids or their derivatives, cationic dendrimers, polyamines, polyamine sugars, amino polysaccharides, oligofectamine, modified polyaminoacids, β-aminoacid-polymers, reversed polyamides, modified polyethylenes, modified acrylates, modified amidoamines, modified polybetaaminoester, dendrimers, polypropylamine dendrimers, poly(amidoamine) PAMAM based dendrimers, polyimines, poly(ethyleneimine), poly(propyleneimine), polyallylamine, polylysine, polyornithine, poly/lysine/ornithine, poly(propylene imine), poly(vinyl amine), poly(2-aminoethyl methacrylate), sugar backbone based polymers, cyclodextrin based polymers, dextran based polymers, chitosan, or silane backbone based polymers, or derivatives, combinations, or mixtures thereof.

In some embodiments, a cellular uptake agent may be linear, branched, or dendrimeric in structure.

In another embodiment a cellular uptake agent may comprise, including but not limited to, at least one or more of the following: polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, or polyarylates, or derivatives, combinations, or mixtures thereof.

In a non-limiting example, a cellular uptake agent comprised of a polymer may comprise, including but not limited to, one or more of the following: poly(caprolactone), ethylene vinyl acetate polymer, poly(lactic acid), poly(L-lactic acid), poly(glycolic acid), poly(lactic acid-co-glycolic acid), poly(L-lactic acid-co-glycolic acid), poly(D,L-lactide), poly(L-lactide), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine, hydroxypropyl methacrylate, polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene or polypropylene, polyalkylene glycols such as poly(ethylene glycol), polyalkylene oxides, polyalkylene terephthalates such as poly(ethylene terephthalate), polyvinyl alcohols, polyvinyl ethers, polyvinyl esters such as poly(vinyl acetate), polyvinyl halides such as poly(vinyl chloride), polyvinylpyrrolidone, polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) or copolymers or mixtures thereof, polydioxanone or its copolymers, polyhydroxyalkanoates, polypropylene fumarate, polyoxymethylene, poloxamers, polyoxamines, poly(ortho)esters, poly(butyric acid), poly(valeric acid), poly(lactide-co-caprolactone), trimethylene carbonate, poly(N-acryloylmorpholine), poly(2-methyl-2-oxazoline), poly(2-ethyl-2-oxazoline), or polyglycerol, or derivatives, combinations, or mixtures thereof.

Non-limiting examples of cellular uptake agents that may be suitable for use with the RNA stabilizing substances of the present invention are described in US Patent Application Pub. No. US 2020/0383922 A1, incorporated herein by reference, as cationic or polycationic compounds that may be used as transfection or complexation agents.

Non-limiting examples of cellular uptake agents that may be suitable for use with the RNA stabilizing substances of the present invention are described in U.S. Pat. Nos. 10,702,600 and 10,933,127, incorporated herein by reference, as nanoparticle formulations, cationic lipid nanoparticles, nanoparticles, liposomes, lipoplexes, lipid nanoparticles (LNPs), lipids, cationic lipids, ionizable lipids, PEG lipids, structural lipids, neutral lipids, non-cationic lipids, therapeutic nanoparticles, polymeric material, polymer-vitamin conjugate, block co-polymer or tri-block co-polymer, surface altering agents, or cationic or polycationic compounds.

Non-limiting examples of cellular uptake agents that may be suitable for use with the RNA stabilizing substances of the present invention are described in U.S. Pat. Nos. 8,058,069 and 9,364,435, incorporated herein by reference, as lipid particles, stable nucleic acid-lipid particle (SNALP), lipids, lipid conjugates, amphipathic lipids, neutral lipids, non-cationic lipids, anionic lipids, cationic lipids, hydrophobic lipids, or sterols.

Non-limiting examples of cellular uptake agents that may be suitable for use with the RNA stabilizing substances of the present invention are described in WO Patent Application Pub. No. WO 2021/156267 A1, incorporated herein by reference, as polymeric carriers, lipidoids or cationic lipidoids, lipid nanoparticles (LNPs), liposomes, lipoplexes, nanoliposomes, lipids, cationic or polycationic lipids, neutral lipids, ionizable lipids, polymer conjugated lipids, cationic or polycationic compounds, cationic or polycationic polymers, cationic or polycationic polysaccharides, cationic or polycationic proteins, or cationic or polycationic peptides.

Non-limiting examples of cellular uptake agents that may be suitable for use with the RNA stabilizing substances of the present invention are described in U.S. Pat. No. 8,367,628, incorporated herein by reference, as lipids, amphoteric lipids, amphoteric liposomes, amphoteric liposomal mixtures, liposomal mixtures, sterols, cationic lipids, chargeable cationic lipids, chargeable anionic lipids, stable anionic lipids, neutral lipids, or mixtures of lipid components with amphoteric properties.

Non-limiting examples of cellular uptake agents that may be suitable for use with the RNA stabilizing substances of the present invention are described in US Patent Application Pub. No. US 2021/0260097 A1, incorporated herein by reference, as nanoparticles, lipid nanoparticles (LNPs), lipids, cationic or ionizable lipids, anionic lipids, neutral lipids, amphipathic lipids, PEGylated lipids, or structural lipids.

Non-limiting examples of cellular uptake agents that may be suitable for use with the RNA stabilizing substances of the present invention are described in US Patent Application Pub. No. US 2021/0261627 A1, incorporated herein by reference, as cationic or polycationic compounds, polymeric carriers, cationic polysaccharides, cationic lipids, polymers, cationic or polycationic polymers, copolymers, blockpolymers, or cationic or polycationic proteins or peptides, which may be used as transfection or complexation agents.

### Containers and Mixing

Chambers may include containers, such as syringes or vials, which may hold a combination of at least one or more RNA stabilizing substance and at least one or more RNA substance and the containers may also contain one or more additional substance, such as at least one or more cellular uptake agents, or one or more buffering agent, or one or more additional RNA stabilizing substances as non-limiting examples.

Furthermore, chambers may include containers, such as syringes or vials, which may hold a combination of at least one or more RNA stabilizing substance and at least one or more RNA substance and the containers may also contain one or more additional substance, such as at least one or more cellular uptake agents or other substances, that are kept separate from the RNA stabilizing substance and the RNA substance until close to the time of use.

As a non-limiting example, a container may hold a combination of materials comprising at least one or more RNA substance and at least one or more RNA stabilizing substance that is ready for injection after removal from the container such as by withdrawal using a syringe and needle. As a non-limiting example, a container, which may be a vial as a non-limiting example, may hold a combination of materials that is a concentrate for injection comprising at least one or more RNA substance and at least one or more RNA stabilizing substance that after dilution is ready for injection upon removal from the container such as by withdrawal using a syringe and needle. As a non-limiting example, the dilution may occur by adding a solution comprising water to a container, which may be a vial, that is partially filled to leave volume for adding and mixing diluting solution. As a non-limiting example, the diluting solution may be 0.9% sodium chloride (normal saline, preservative-free). As a non-liming example, the container may be a multi-dose container, which may be a multi-dose vial, containing at least two doses or containing at least 5 doses or containing at least 10 doses or containing at least 15 doses. As a non-liming example, the container may be a multi-dose vial that is at least 2 ml size or that is at least 5 ml size or that is at least 10 ml size or that is at least 20 ml size or that is at least 30 ml size.

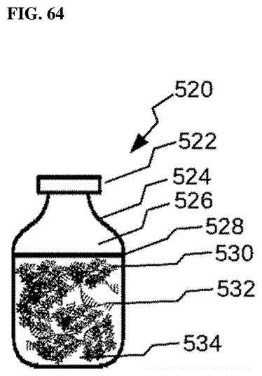

As a non-limiting example, a vial 520, as shown in FIG. 64, may have a seal 522 affixed to a container 524 forming an enclosed space 526 that is at least partially filled to a predetermined quantity or level 528 with liquid material 534 comprising at least one RNA stabilizing substance 530 (depicted as dots in FIG. 64) at least one RNA substance 532 (depicted as the wave lines and straight lines in FIG. 64). As a non-limiting example, seal 522 may comprise an elastomeric reseal that may be penetrated by a needle (not shown) affixed to syringe (not shown), enabling liquid material 534 to be at least partially withdrawn from vial 520 and transferred to the syringe using techniques known to those skilled in the art. A non-limiting alternative example use of vial 520 is to have the syringe at least partially contain a material, such as normal saline as a non-limiting example, suitable for diluting liquid material 534 comprising at least RNA stabilizing substance 530 and RNA substance 532 by injecting the material from the syringe (not shown) into vail 520 prior to withdrawing at least part of the combined, and as a non-limiting example, mixed, materials from vial 520 into the syringe. One or more syringes may be used to withdraw materials from vial 520. Other devices besides syringes may be used to withdraw materials, non-limiting examples include pumping devices and devices that pressurize enclosed space 526 of vial 520.

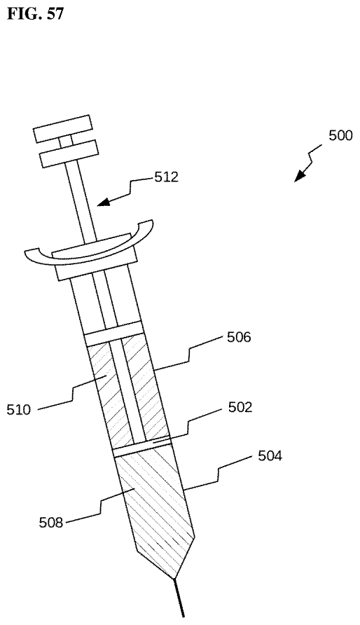

As a non-limiting example, a multi-chamber syringe 500, as shown in FIG. 57, with a breakable seal 502 between chambers may have a first mixture 508 including at least one or more RNA stabilizing substance, at least one or more RNA substance, and at least one or more cellular uptake agents, such as lipids, in one chamber 504 and an aqueous solution 510, such as water, in a second chamber 506. At time of use the seal 502 between the chambers 504 and 506 is broken and the contents of both chambers 504 and 506 are mixed to induce at least one or more cellular uptake agent, such as lipids, to combine with one or more RNA substance prior to use. For example, the seal 502 may be broken by advancing or turning a plunger assembly 512 or portion thereof to puncture a membrane or otherwise enable mixing. In another non-limiting example, at least one or more RNA substance and at least one or more RNA stabilizing substance are in one chamber and both an aqueous solution, such as water, and at least one or more cellular uptake agent, such as a polymer, are in a second chamber with a breakable seal between the chambers. At time of use the seal is broken and the contents of the two chambers are mixed to induce at least one or more cellular uptake agent, such as a polymer, to combine with one or more RNA substance prior to use.

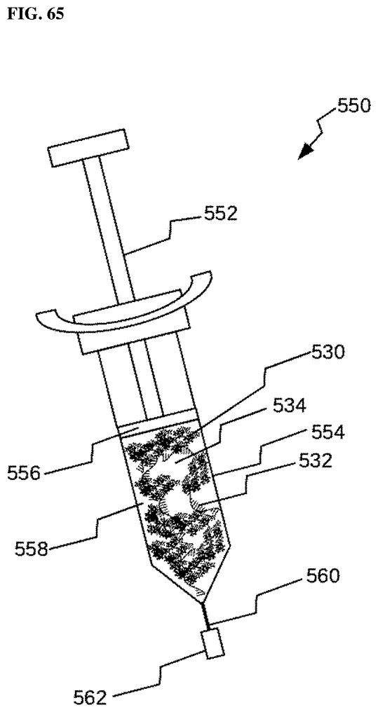

As a non-limiting example, single chamber syringe 550, is shown in FIG. 65, As a non-limiting example, a syringe 550, may have plunger assembly 552 slidably moving relative to syringe container 554 with sliding seal element 556 as part of plunger assembly 552 forming an enclosed space 558 that is at least partially filled with liquid material 534 comprising at one least RNA stabilizing substance 530 (depicted as dots in FIG. 65) and at least one RNA substance 532 (depicted as the wave lines and straight lines in FIG. 65). Delivery port 560 fluidically communicates with enclosed space 558 and may, as non-limiting examples, be a needle or an opening (not shown) syringe 550 to which a needle or other delivery component attaches. Port sealing element 562 substantially retains liquid material 534 inside enclosed container space 558 after liquid material 534 is loaded into enclosed container space such as during prefilling syringe 550 during manufacturing or other preparation. Port sealing element 562 may, as non-limiting examples, seal port 560 by surrounding the outside of the port or by plugging the inside of one or more fluid channels in port 560. Liquid material 534 is transferred from the syringe using techniques known to those skilled in the art by operating plunger assembly 552.

In other embodiments of the present invention, containers may be embedded complexes comprising at least one RNA stabilizing substance and at least one RNA substance that remain substantially non-communicative across at least one boundary until interaction with a biologic material, as a non-limiting example the biologic material may be fluid or tissue in a living organism, alters the embedded complex so that at least one RNA substance moves from the embedded complex. As a non-limiting example, the embedded complex may be implanted in a living organism and upon being altered by biologic material of the living organism at least one RNA substance at least partially moves from the embedded complex into at least one tissue of the living organism. As a non-limiting example, one or more other substances besides RNA substance may at least partially move from the embedded complex into at least one tissue. As non-limiting examples, the substances besides RNA substances may comprise at least one of RNA stabilizing substances, cellular uptake agents, materials antagonistic to cellular activity either alone or in combination with metabolic processes influenced by one or more RNA substance, and substances benefiting cellular activity either alone or in combination with metabolic process influenced by one or more RNA substance. As a non-limiting example, the embedded complex may deliver one or more RNA substances that produce a response in neoplasms that alter, inhibit, or terminate metabolic activity of at least one neoplastic cell. As non-limiting example the embedded complex may be implanted in at least the vicinity or at least partially in a neoplasm or where communication between at least one RNA substance and at least one neoplasm may occur through biologic transport processes such through fluid transfer, which may include transfer through the blood system or through the lymph system. As a non-limiting example, the embedded complex may comprise at least one RNA substance that affects the survival or growth of at least one non-malignant neoplasm. As a non-limiting example, the embedded complex may comprise at least one RNA substance that affects the survival or growth of at least one malignant neoplasm.

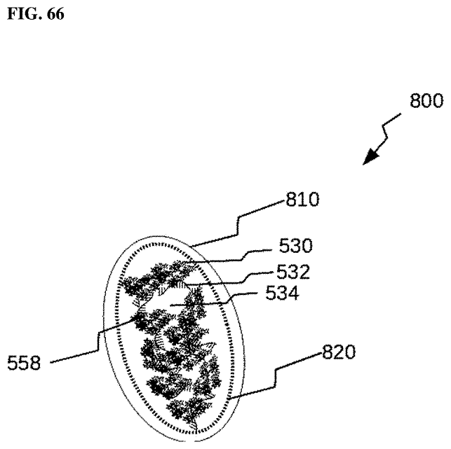

As a non-limiting example, embedded complex 800, is shown in FIG. 66, As a non-limiting example, embedded complex 800, may have a packaging layer 810 that that protects the implantable material during shipping, storage, and provides a sterile barrier. Polymeric material 820 form an enclosed space 558 that at least partially contains internal material 534 comprising at least one RNA substance 532 (depicted as the wave lines and straight lines in FIG. 66). Polymeric material 820 may comprise an RNA stabilizing substance, as a non-limiting example polymeric material 820 may comprise one or more RNA stabilizing polymer. Polymeric material 820 may degrade when exposed to biologic materials, such biologic fluids and such degradation may allow internal material 534 to enter one or more tissues in which embedded complex 800 is embedded, such by implanted in one or more tissues. The tissues in which embedded complex 800 is embedded may be from implanting into animal tissue and as non-limiting examples, the animal tissues may be one or more mammalian tissues. As a non-limiting example the mammalian tissues and may be one or more human tissues.

Embedded complex 800 may have diffusion controlling barrier in addition to or instead of polymeric material that degrades when exposed to biologic materials.

As another non-limiting example, embedded complex 800 may be a container such as a tablet, lozenge, capsule, gel capsule, or other container that contacts biologic fluids without being implanted. As non-limiting examples, embedded complex 800 may be ingested orally, be a suppository, or be at least part of an inhalable material such as a mist or droplets produced by a nebulizer.

The containers that may hold a combination comprising at least one or more RNA stabilizing substance and at least one or more RNA substance may be made of any materials suitable for storing and shipping at least one RNA stabilizing substance and at least one RNA substance including, but not limited to, glass, metal, ceramic, plastic or other polymeric material that does not degrade or modify the container's contents or be degraded or modified by the container's contents. The container may have an access port that is penetrated or removed to access the interior of the container including accessing at least part of the contents of the container. The containers may have an access port, such as a screw lid, removable tab, resealable plug or cap or closure that may be penetrated, such as by a hollow tube, such as a hollow needle, to access the interior of the container either to add one or more materials to the contents of the container or to remove at least part of the contents from the interior of the container.

The containers may be used to add materials to the at least one or more RNA stabilizing substance and at least one or more RNA substance. As a non-limiting example, a syringe containing a substance comprising at least one or more lipid may have at least part of its contents transferred to the interior of the container through the access port such as by using a hollow needle penetrating a resealing closure. As another non-limiting example, the syringe may contain an aqueous solution, such as water, that is at least partially transferred to a container having contents comprising at least one RNA stabilizing substance and at least one RNA substance.

The containers, described above, may also hold a combination comprising at least one or more RNA stabilizing substance, at least one or more RNA substance, and at least one or more cellular uptake agent. The containers, described above, may be made of any materials suitable for storing and shipping at least one RNA stabilizing substance, at least one RNA substance and at least one cellular uptake agent including, but not limited to, glass, metal, ceramic, plastic or other polymeric material that does not degrade or modify the container's contents or be degraded or modified by the container's contents.

The containers, described above, may be used to add materials to the at least one or more RNA stabilizing substance, at least one or more RNA substance, and at least one or more cellular uptake agent. As another non-limiting example, the syringe may contain an aqueous solution, such as water, that is at least partially transferred to a container having contents comprising at least one RNA stabilizing substance and at least one RNA substance and at least one cellular uptake agent.

### Uses and Applications:

In one method of use a combination comprising at least one or more RNA stabilizing substance and at least one or more RNA substance is a combination stored in a chamber at a temperature greater than or equal to about −60° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about −40° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about −20° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about −10° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about −5° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about 0° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about 2° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about 4° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about 6° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about 8° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about 10° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about 15° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about 20° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about 25° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

In another method of use a combination comprising at least one or more RNA stabilizing substance and at least one or more RNA substance is a combination stored in a chamber at a temperature greater than or equal to about 30° C. for a duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months and 24 months and the maximum time is greater than the minimum time and may be at least one of 6 hours, 24 hours, 48 hours, 72 hours, 100 hours 7 days, 14 days, 30 days, 60 days, 3 months, 6 months, 12 months, 18 months, 24 months, 48 months, 5 years, 10 years, and 20 years.

Embodiments of the foregoing methods of use include using one or more RNA stabilizing substance mixed with one or more RNA substance.

In one method of use a combination comprising at least one or more RNA stabilizing substance and at least one or more RNA substance is a combination stored at a temperature less than the melting point of the combination of substances.

In one method of use a combination comprising at least one or more RNA stabilizing substance and at least one or more RNA substance is a combination stored at a temperature less than the melting point of the combination of substances.

In one method of use a combination comprising at least one or more RNA stabilizing substance and at least one or more RNA substance and at least one or more cellular uptake agent is a combination stored at a temperature less than the melting point of the combination of substances.

In one method of use a combination comprising at least one or more RNA stabilizing substance and at least one or more RNA substance and at least one or more cellular uptake agent is a combination stored at a temperature less than the melting point of the combination of substances.

### Applications and Methods of Use

One embodiment of the present invention is the method whereby one or more RNA stabilizing substance may be combined, such as by mixing, with at least one or more RNA substance to produce a mixture comprising at least one or more RNA stabilizing substance and at least one or more RNA substance. As a non-limiting example one or more RNA substance may be mixed with one or more RNA stabilizing substance.

Another embodiment of the present invention is the method whereby one or more RNA stabilizing substance may be combined, such as by mixing, with at least one or more RNA substance to produce a mixture comprising at least one or more RNA substance and at least one or more RNA stabilizing substance at one or more of the RNA stabilizing substance concentrations within the stabilizing substance molar concentration range list or in the stabilizing substance molar concentration list or in the stabilizing substance weight percent concentration list as described herein. These same methods may be used to combine other substances, including, but not limited to, cellular uptake agents, water, buffering agents or additional RNA stabilizing substances, with one or more RNA substances and one or more RNA stabilizing substances to produce a mixture comprising one or more RNA substance, one or more RNA stabilizing substance, and one or more cellular uptake agent or additional other substances.

One embodiment of the present invention is the method whereby one or more RNA stabilizing substance may be combined, such as by mixing, with at least one or more RNA substance to produce a composition comprising at least one or more RNA substance and at least one or more RNA stabilizing substance. Another embodiment of the present invention is the method whereby one or more RNA stabilizing substance may be combined, such as by mixing, with one or more RNA substance to produce a composition comprising at least one or more RNA substance and at least one or more RNA stabilizing substance at one or more of the RNA stabilizing substance concentrations within the stabilizing substance molar concentration range list or in the stabilizing substance molar concentration list or in the stabilizing substance weight percent concentration list as described herein. These same methods may be used to combine other substances, including, but not limited to cellular uptake agents, water, buffering agents or additional RNA stabilizing substances, with one or more RNA substances and one or more RNA stabilizing substances to produce a composition comprising one or more RNA substance, one or more RNA stabilizing substance, and one or more cellular uptake agent or additional other substances.

One embodiment of the present invention is the method whereby one or more RNA stabilizing substance may be combined with one or more RNA substance such as by, including but not limited to, mixing, pipetting, blending, stirring, inverting, submerging, vortexing, shaking, lyophilizing, vaporizing, or sublimating such that at least one or more RNA stabilizing substance is at least intimately associated with or at least partially contacting or at least partially encapsulating at least one or more RNA substance.

One embodiment of the present invention is the method whereby one or more RNA stabilizing substance may be combined with one or more RNA substance by mixing the substances using known methods. These methods include, but are not limited to, stirring, fluid flow agitation, vortexing, inverting, pipetting, blending, multiple channel fluidics, low shear blending, microfluidic mixing, or using static mixers. These same methods may be used to combine other substances, including, but not limited to cellular uptake agents, water, buffering agents or additional RNA stabilizing substances, with one or more RNA substances and one or more RNA stabilizing substances.

Embodiments of methods described herein may be independent of the order in which each substance may be combined or mixed together. As a non-limiting example one or more RNA stabilizing substance may be combined with one or more RNA substance or one or more RNA substance may be combined with one or more RNA stabilizing substance by the same method.

One embodiment of the present invention is the method whereby one or more RNA stabilizing substances may be combined with at least one or more RNA substance in a container comprising glass, plastic, ceramic, an elastomer, a polymer, or metal.

One embodiment of the present invention is the method whereby one or more RNA stabilizing substances may be combined with at least one or more RNA substance and introduced into a container comprising glass, plastic, ceramic, an elastomer, a polymer, or metal.

In one embodiment, single doses of a stabilized RNA composition may be packaged and sealed. In one embodiment, multiple doses of a stabilized RNA composition may be packaged and sealed in one packaging unit. As a non-limiting example, single doses or multiple doses may be packaged in chambers.

In another embodiment of the present invention one or more RNA stabilizing substance may also be used in conjunction with lyophilization of at least one or more RNA substance. In one embodiment of the present invention one or more RNA stabilizing substance may not be used in conjunction with lyophilization of at least one or more RNA substance.

As used herein, stabilized RNA compositions or RNA stabilizing compositions, may be one or more composition, combination, or mixture comprising at least one or more RNA stabilizing substance and at least one or more RNA substance as described herein. Stabilized RNA compositions or RNA stabilizing compositions may also comprise one or more or more additional substances, including but not limited to, cellular uptake agents, water, buffering agents, inorganic salts, chelating agents, or additional RNA stabilizing substances as non-limiting examples.

In a further aspect, the present invention further provides the use of the inventive method in the manufacture of a pharmaceutical composition.

In one embodiment of the present invention, one or more RNA stabilizing compositions may be used in the manufacture of a pharmaceutical composition.

According to yet another aspect of the present invention, a pharmaceutical composition may be provided, wherein a pharmaceutical composition may comprise one or more RNA stabilizing compositions as described herein.

In one embodiment a pharmaceutical composition may be used to treat, prevent, cure, or diagnose one or more disease or improve or prolong the health of humans, plants, or animals, including non-human primates, vertebrate animals, and non-vertebrate animals.

In one embodiment a pharmaceutical composition may comprise one or more composition, combination, or mixture comprising at least one or more RNA stabilizing substance and at least one or more RNA substance as described herein. In another embodiment a pharmaceutical composition may comprise one or more composition, combination, or mixture comprising at least one or more RNA stabilizing substance, at least one or more RNA substance, and at least one or more cellular uptake agent as described herein.

In another embodiment, a pharmaceutical composition may comprise one or more additional pharmaceutically acceptable ingredient, such as a pharmaceutically acceptable carrier or vehicle.

In another embodiment one or more RNA substance within a pharmaceutical composition may comprise at least one or more pharmaceutically active ingredients.

In one embodiment a pharmaceutical composition may comprise at least one or more pharmaceutically active RNA component. In one embodiment a pharmaceutical composition may comprise at least one or more biologically active RNA component.

In another embodiment a pharmaceutical composition may comprise one or more non-RNA pharmaceutically active component. Wherein a non-RNA pharmaceutically active component may be a compound that has a therapeutic effect against a particular medical indication, such as, but not limited to, cancer diseases, autoimmune disease, allergies, infectious diseases or a further disease, as non-limiting examples. Non-limiting examples of such compounds may include, but are not limited to: peptides or proteins, (therapeutically active) low molecular weight organic or inorganic compounds (molecular weight less than 5,000), sugars, antigens or antibodies, therapeutic agents already known in the art, antigenic cells, antigenic cellular fragments, cellular fractions, modified, attenuated or de-activated pathogens (e.g. virus, bacteria, fungus, protozoa, plasmodium, or mycobacterium), wherein a pathogen may be attenuated or deactivated chemically, by irradiation, mutation, serial passage, or other known method.

In one embodiment one or more pharmaceutical compositions may be administered orally, sublingually, transdermally, ophthalmically, parenterally, subcutaneous, intravenous, intramuscular, by inhalation, topically, rectally, nasally, buccally, vaginally, or via an implant. The term parenteral or parenterally as used herein includes, but is not limited to, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

In one method of use one or more pharmaceutical compositions may be administered orally, sublingually, transdermally, ophthalmically, parenterally, subcutaneous, intravenous, intramuscular, by inhalation, topically, rectally, nasally, buccally, vaginally, or via an implant. The term parenteral or parenterally as used herein includes, but is not limited to, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

In one method of use one or more RNA stabilizing composition as described herein may be used to produce a pharmaceutical composition comprising at least one or more RNA substance and at least one or more RNA stabilizing substance.

In one method of use one or more RNA stabilizing composition as described herein may be used to produce a pharmaceutical composition comprising at least one or more RNA substance, at least one or more RNA stabilizing substance, and at least one or more cellular uptake agent.

In one method of use one or more RNA stabilizing composition as described herein may be combined with one or more pharmaceutical composition comprising at least one or more RNA substance and at least one or more RNA stabilizing substance.

In one method of use one or more RNA stabilizing composition as described herein may be combined with one or more pharmaceutical composition comprising at least one or more RNA substance, at least one or more RNA stabilizing substance, and at least one or more cellular uptake agent.

In one embodiment one or more RNA stabilizing composition as described herein may be combined with one or more pharmaceutical composition comprising at least one or more RNA substance and at least one or more RNA stabilizing substance.

In one embodiment one or more RNA stabilizing composition as described herein may be combined with one or more pharmaceutical composition comprising at least one or more RNA substance, at least one or more RNA stabilizing substance, and at least one or more cellular uptake agent.

In one embodiment one or more pharmaceutical composition may comprise one or more medicament, vaccine, therapeutic agent, or biostimulant.

In a further aspect, the present invention further provides the use of the inventive method in the manufacture of a medicament, vaccine, or therapeutic agent.

In one embodiment of the present invention, one or more RNA stabilizing compositions may be used in the manufacture of a medicament, vaccine, or therapeutic agent.

According to yet another aspect of the present invention, a medicament, vaccine, or therapeutic agent may be provided, wherein a medicament, vaccine, or therapeutic agent may comprise one or more RNA stabilizing compositions as described herein.

In one embodiment a medicament, vaccine, or therapeutic agent may comprise one or more composition, combination, or mixture comprising at least one or more RNA stabilizing substance and at least one or more RNA substance as described herein. In another embodiment a medicament, vaccine, or therapeutic agent may comprise one or more composition, combination, or mixture comprising at least one or more RNA stabilizing substance, at least one or more RNA substance, and at least one or more cellular uptake agent as described herein.

In another embodiment, a medicament, vaccine, or therapeutic agent may comprise one or more additional pharmaceutically acceptable ingredient, such as a pharmaceutically acceptable carrier or vehicle.

In another embodiment one or more RNA substance within a medicament, vaccine, or therapeutic agent may comprise at least one or more pharmaceutically active ingredients.

In one embodiment a medicament, vaccine, or therapeutic agent may comprise one or more pharmaceutically active RNA component. In one embodiment a medicament, vaccine, or therapeutic agent may comprise one or more biologically active RNA component.

In another embodiment a medicament, vaccine, or therapeutic agent may comprise one or more non-RNA pharmaceutically active component. Wherein a non-RNA pharmaceutically active component may be a compound that has a therapeutic effect against a particular medical indication, such as, but not limited to, cancer diseases, autoimmune disease, allergies, infectious diseases or a further disease, as non-limiting examples. Non-limiting examples of such compounds may include, but are not limited to: peptides or proteins, (therapeutically active) low molecular weight organic or inorganic compounds (molecular weight less than 5,000), sugars, antigens or antibodies, therapeutic agents already known in the art, antigenic cells, antigenic cellular fragments, cellular fractions, modified, attenuated or de-activated pathogens (e.g. virus, bacteria, fungus, protozoa, plasmodium, or mycobacterium), wherein a pathogen may be attenuated or deactivated chemically, by irradiation, mutation, serial passage, or other known method.

In one embodiment one or more medicament, vaccine, or therapeutic agent, may be administered orally, sublingually, transdermally, ophthalmically, parenterally, subcutaneous, intravenous, intramuscular, by inhalation, topically, rectally, nasally, buccally, vaginally, or via an implant. The term parenteral or parenterally as used herein includes, but is not limited to, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

In one method of use one or more medicament, vaccine, or therapeutic agent, may be administered orally, sublingually, transdermally, ophthalmically, parenterally, subcutaneous, intravenous, intramuscular, by inhalation, topically, rectally, nasally, buccally, vaginally, or via an implant. The term parenteral or parenterally as used herein includes, but is not limited to, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

In one method of use one or more RNA stabilizing composition as described herein may be used to produce a medicament, vaccine, or therapeutic agent comprising at least one or more RNA substance and at least one or more RNA stabilizing substance.

In one method of use one or more RNA stabilizing composition as described herein may be used to produce a medicament, vaccine, or therapeutic agent comprising at least one or more RNA substance, at least one or more RNA stabilizing substance, and at least one or more cellular uptake agent.

In one method of use one or more RNA stabilizing composition as described herein may be combined with a medicament, vaccine, or therapeutic agent comprising at least one or more RNA substance and at least one or more RNA stabilizing substance.

In one method of use one or more RNA stabilizing composition as described herein may be combined with a medicament, vaccine, or therapeutic agent comprising at least one or more RNA substance, at least one or more RNA stabilizing substance, and at least one or more cellular uptake agent.

In one embodiment one or more RNA stabilizing composition as described herein may be combined with one or more medicament, vaccine, or therapeutic agent comprising at least one or more RNA substance and at least one or more RNA stabilizing substance.

In one embodiment one or more RNA stabilizing composition as described herein may be combined with one or more medicament, vaccine, or therapeutic agent comprising at least one or more RNA substance, at least one or more RNA stabilizing substance, and at least one or more cellular uptake agent.

In a further aspect, the present invention further provides the use of the inventive method in the manufacture of one or more biostimulant composition, including, but not limited to one or more of the following: plant biostimulant, crop biostimulant, agricultural biostimulant, agricultural treatment, soil treatment, horticultural biostimulant, horticultural treatment, microbial biostimulant, bacterial biostimulant, fungal biostimulant, antifungal, antibiotic, antiviral, viricide, microbial treatment, microbial disinfectant, biocide, microbicide, pesticide, insecticide, or insect biostimulant.

As used herein a biostimulant is one or more substance delivered to an organism (such as plants, algae, fungi, or bacteria as non-limiting examples) with the aim to alter or enhance the characteristics, nutrition efficiency, stress tolerance (including biotic or abiotic stress tolerance), or organism quality traits.

As used herein a biostimulant composition is a composition, combination, or mixture comprised of at least one or more biostimulants.

In one embodiment of the present invention, one or more RNA stabilizing compositions may be used in the manufacture of one or more biostimulant composition, including, but not limited to one or more of the following: plant biostimulant, crop biostimulant, agricultural biostimulant, agricultural treatment, soil treatment, horticultural biostimulant, horticultural treatment, microbial biostimulant, bacterial biostimulant, fungal biostimulant, antifungal, antibiotic, antiviral, viricide, microbial treatment, microbial disinfectant, biocide, microbicide, pesticide, insecticide, or insect biostimulant.

According to yet another aspect of the present invention, a biostimulant composition may be provided, wherein a biostimulant composition may comprise one or more RNA stabilizing compositions as described herein.

In one embodiment a biostimulant composition may comprise one or more composition, combination, or mixture comprising at least one or more RNA stabilizing substance and at least one or more RNA substance as described herein. In another embodiment a biostimulant composition may comprise one or more composition, combination, or mixture comprising at least one or more RNA stabilizing substance, at least one or more RNA substance, and at least one or more cellular uptake agent as described herein.

In another embodiment, a biostimulant composition may comprise one or more additional biologically, environmentally, or agriculturally acceptable ingredient, such as a biologically, environmentally, or agriculturally acceptable carrier or vehicle.

In another embodiment one or more RNA substance within a biostimulant composition may comprise at least one or more biologically active ingredients.

In one embodiment a biostimulant composition may comprise one or more biologically active RNA component.

In another embodiment a biostimulant composition may comprise one or more non-RNA biologically active component.

In one embodiment one or more biostimulant compositions may be administered via spray, powder, aerosol, mist, solution, water additive, soil additive, fertilizer additive, liquid, tablet, or other known methods.

In one method of use one or more biostimulant compositions may be administered via spray, powder, aerosol, mist, solution, water additive, soil additive, fertilizer additive, liquid, tablet, or other known methods.

In one method of use one or more RNA stabilizing composition as described herein may be used to produce a biostimulant composition comprising at least one or more RNA substance and at least one or more RNA stabilizing substance.

In one method of use one or more RNA stabilizing composition as described herein may be used to produce a biostimulant composition comprising at least one or more RNA substance, at least one or more RNA stabilizing substance, and at least one or more cellular uptake agent.

In one method of use one or more RNA stabilizing composition as described herein may be combined with a biostimulant composition comprising at least one or more RNA substance and at least one or more RNA stabilizing substance.

In one method of use one or more RNA stabilizing composition as described herein may be combined with a biostimulant composition comprising at least one or more RNA substance, at least one or more RNA stabilizing substance, and at least one or more cellular uptake agent.

In one embodiment one or more RNA stabilizing composition as described herein may be combined with one or more biostimulant composition comprising at least one or more RNA substance and at least one or more RNA stabilizing substance.

In one embodiment one or more RNA stabilizing composition as described herein may be combined with one or more biostimulant composition comprising at least one or more RNA substance, at least one or more RNA stabilizing substance, and at least one or more cellular uptake agent.

In a further aspect, the present invention further provides the use of the inventive method in the manufacture of one or more implants, wherein an implant may be implanted, attached or otherwise contacting tissues, skin, blood, fluid, or cells of a living organism, such as humans, plants, or animals as non-limiting examples.

In one embodiment of the present invention, one or more RNA stabilizing compositions may be used in the manufacture of one or more implant, wherein an implant may be implanted, attached or otherwise contacting tissues, skin, blood, fluid, or cells of a living organism, such as humans, plants, or animals as non-limiting examples.

According to yet another aspect of the present invention, an implant may be provided, wherein an implant may comprise one or more RNA stabilizing compositions as described herein.

In one embodiment an implant may be used to treat, prevent, cure, or diagnose one or more disease or improve or prolong the health of humans, plants, or animals, including non-human primates, vertebrate animals, and non-vertebrate animals.

In one embodiment an implant may comprise one or more composition, combination, or mixture comprising at least one or more RNA stabilizing substance and at least one or more RNA substance as described herein. In another embodiment an implant may comprise one or more composition, combination, or mixture comprising at least one or more RNA stabilizing substance, at least one or more RNA substance, and at least one or more cellular uptake agent as described herein.

In another embodiment, an implant may comprise one or more additional pharmaceutically, biologically, environmentally, or agriculturally acceptable ingredient, such as a pharmaceutically, biologically, environmentally, or agriculturally acceptable carrier or vehicle.

In another embodiment one or more RNA substance within an implant may comprise at least one or more pharmaceutically active ingredients. In another embodiment one or more RNA substance within an implant may comprise at least one or more biologically active ingredients.

In one embodiment an implant may comprise one or more pharmaceutically active RNA component. In one embodiment an implant may comprise one or more biologically active RNA component.

In another embodiment an implant may comprise one or more non-RNA pharmaceutically or biologically active component. Wherein a non-RNA pharmaceutically active component may be a compound that has a therapeutic effect against a particular medical indication, such as, but not limited to, cancer diseases, autoimmune disease, allergies, infectious diseases or a further disease, as non-limiting examples. Non-limiting examples of such compounds may include, but are not limited to: peptides or proteins, (therapeutically active) low molecular weight organic or inorganic compounds (molecular weight less than 5,000), sugars, antigens or antibodies, therapeutic agents already known in the art, antigenic cells, antigenic cellular fragments, cellular fractions, modified, attenuated or de-activated pathogens (e.g. virus, bacteria, fungus, protozoa, plasmodium, or mycobacterium), wherein a pathogen may be attenuated or deactivated chemically, by irradiation, mutation, serial passage, or other known method.

In one embodiment one or more implants may be implanted or administered orally, sublingually, transdermally, ophthalmically, parenterally, subcutaneous, intravenous, intramuscular, by inhalation, topically, rectally, nasally, buccally, vaginally, or via implant. The term parenteral or parenterally as used herein includes, but is not limited to, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

In one method of use one or more implants may be implanted or administered orally, sublingually, transdermally, ophthalmically, parenterally, subcutaneous, intravenous, intramuscular, by inhalation, topically, rectally, nasally, buccally, vaginally, or via implant. The term parenteral or parenterally as used herein includes, but is not limited to, subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

In one embodiment an implant may be one or more device (such as a pump, implanted pump, reservoir, or port) or one or more material or matrix (such as a dissolvable material or matrix, biodegradable material or matrix, gel, or other material or matrix capable of mass transport such as by diffusion) located internally or externally comprising one or more substance (such as a solid, liquid, fluid, biostimulant, medicament, vaccine, or therapeutic agent) that enters into an organism, and is thereby implanted, attached or otherwise contacting tissues, skin, blood, fluid, or cells of a living organism, such as humans, plants, or animals as non-limiting examples.

In one embodiment one or more implant, may be implanted or administered via patch, transdermal patch, suppository, orally, swallowed, injected, sprayed, inhaled, aerosol, powder, mist, water additive, soil additive, fertilizer additive, liquid, tablet, or other known methods.

In one method of use one or more implant may be implanted or administered via patch, transdermal patch, suppository, orally, swallowed, injected, sprayed, inhaled, aerosol, powder, mist, water additive, soil additive, fertilizer additive, liquid, tablet, or other known methods.

In one method of use one or more RNA stabilizing composition as described herein may be used to produce an implant comprising at least one or more RNA substance and at least one or more RNA stabilizing substance.

In one method of use one or more RNA stabilizing composition as described herein may be used to produce an implant comprising at least one or more RNA substance, at least one or more RNA stabilizing substance, and at least one or more cellular uptake agent.

In one method of use one or more RNA stabilizing composition as described herein may be combined with one or more implant comprising at least one or more RNA substance and at least one or more RNA stabilizing substance.

In one method of use one or more RNA stabilizing composition as described herein may be combined with one or more implant comprising at least one or more RNA substance, at least one or more RNA stabilizing substance, and at least one or more cellular uptake agent.

In one embodiment one or more RNA stabilizing composition as described herein may be combined with one or more implant comprising at least one or more RNA substance and at least one or more RNA stabilizing substance.

In one embodiment one or more RNA stabilizing composition as described herein may be combined with one or more implant comprising at least one or more RNA substance, at least one or more RNA stabilizing substance, and at least one or more cellular uptake agent.

In one embodiment one or more implant may comprise one or more pharmaceutical composition or may be used in conjunction with one or more pharmaceutical composition. In one embodiment one or more implant may comprise one or more medicament, vaccine, or therapeutic agent or may be used in conjunction with one or more medicament, vaccine, or therapeutic agent. In one embodiment one or more implant may comprise one or more biostimulant or biostimulant composition or may be used in conjunction with one or more biostimulant or biostimulant composition.

In one embodiment an embedded complex may be an implant.

In another method of use one or more RNA stabilizing composition as described herein may be used for one or more of the following applications, including, but not limited to, treating a disease, preventing a disease, or producing a cellular response in one or more of the following organisms or cells, which may include but are not limited to: humans, primates, animals, vertebrate animals, eukaryotic cells, eukaryotes, protozoa, prokaryotic cells, plant cells, plants, fungal cells, fungi, insect cells, insects, bacterial cells, bacteria, mycoplasma, protozoa, plasmodium, or mammalian cells, including but not limited to the cells of primates, animals, vertebrate animals, and the cells of humans.

In one method of use one or more RNA stabilizing composition as described herein may be used for in vivo, in vitro, in situ, or ex vivo applications. Such applications may include, but are not limited to, one or more of the following: agricultural applications, agricultural treatment, fertilizer, biostimulant, veterinary applications, animal treatment, pharmaceutical applications, human treatment, therapeutic applications, soil treatment, pesticide, herbicide, bacterial treatment, fungal treatment, antiviral, antibiotic, antifungal, antimicrobial, plant applications, plant treatment, antibody production, protein expression applications, insect treatment, insecticide, vaccine production, therapeutic agent production, implant production, embedded complex production, drug production, or medicament production, or combinations thereof.

In another method of use one or more RNA stabilizing composition as described herein may be used for one or more of the following applications, including but not limited: syringe, prefilled syringe, injection, nasal spray, transdermal patch, eye drop, oral spray, aerosol, inhaler, nebulizer, oral tablet, pill, sublingual tablet, sublingual drop, suppository, mucosal spray, cream, lozenge, lotion, balm, syrup, ointment, fertilizer, biostimulant, implant, solution, powder, water additive, mist, spray, powder, or tablet.

In another method of use one or more RNA stabilizing composition as described herein may be used in composition that also comprises a cellular uptake agent and used as a mucosal spray. Wherein, a mucosal spray may be any container that can be squeezed, pressurized, or applied in such a manner to aerosolize, spray, mist, aspirate, drop, squirt, apply, administer, or direct a combination comprising at least one RNA stabilizing substance and at least one or more RNA substance combined in a mixture comprising at least one cellular uptake agent into or onto a mucosal surface such as a nasal passage, airway, throat, lung, eye, or other mucosal surface within a human, primate, animal, or vertebrate animal.

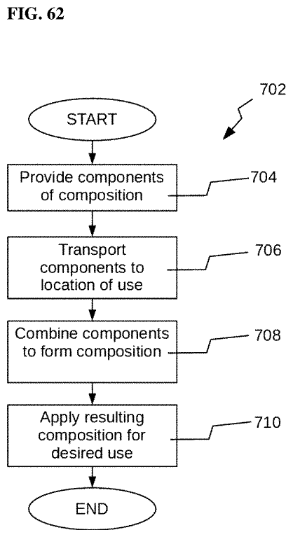

FIG. 62 is a flowchart that summarizes a process 702 for producing and using an RNA product in accordance with the present invention. The process 702 is initiated by providing (704) components of the RNA product or composition. The components may be obtained separately or may be provided as part of a kit or in a preloaded, multi-chamber syringe as described above, among other possibilities. Moreover, the composition, components, or combinations of components, may be provided in bottles, containers, vials, tubes, syringes, blisters, capsules, cartridges, or other packaging methods. The components may then be stored at the location of use or transported (706) to the location of use. Depending on the specific implementation, the components or some of the components may be refrigerated, frozen, or otherwise maintained in a temperature-controlled environment during transportation and storage.

When ready for use, the components can be combined (708) to yield the desired composition. For example, the individual components may be combined in a bottle, vial, or other container by adding the individual components to the container, e.g., by pouring, using a pipette, syringe, or the like, by adding lyophilized pellets, by measuring powders, or by any other suitable method. In certain implementations, the components may be mixed by breaking a breakable seal of a multi-compartment container such as a multi-chamber syringe. Finally, the resulting composition may be applied (710) for the desired use. As otherwise noted herein, the stabilized RNA products of the present invention may be utilized in a variety of fields such as therapeutics, diagnostics, or agriculture. In addition, the product may be packaged and distributed as a medicament, a therapeutic, vaccine, biostimulant, embedded complex, or implant.

Moreover, in the case of pharmaceutical compositions, the product may be packaged and distributed for administration orally, sublingually, transdermally, ophthalmically, parenterally, subcutaneously, intravenously, intramuscularly, by inhalation, topically, rectally, nasally, buccally, vaginally, or via an implant as otherwise described herein. The RNA product may be used for a variety of applications including treating a disease, preventing a disease, or producing a cellular response as otherwise described herein. The product may be used in in vivo, in vitro, in situ, or ex vivo applications. Such applications may include agricultural applications, agricultural treatment, fertilizer, biostimulant, veterinary applications, animal treatment, pharmaceutical applications, human treatment, therapeutic applications, soil treatment, pesticide, herbicide, bacterial treatment, fungal treatment, antiviral, antibiotic, antifungal, antimicrobial, plant applications, plant treatment, antibody production, protein expression applications, insect treatment, insecticide, vaccine production, therapeutic agent production, implant production, embedded complex production, drug production, medicament production, or combinations thereof. Accordingly, applying the resulting composition for the desired use will vary depending on the nature of the composition and the intended use among other things.

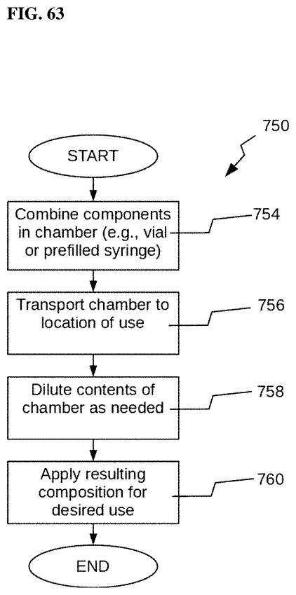

FIG. 63 is a flowchart that summarizes a process 750 for producing and using an RNA product in accordance with the present invention. The process 750 is initiated by providing (754) components of the RNA product or composition and combining them in a chamber or combining them and adding the combination to a chamber. The chamber may be any suitable chamber and may be, as non-limiting examples, a single use or multiuse vial. Moreover, the chamber may be, as non-limiting examples, bottles, containers, vials, tubes, syringes (including prefilled or single use syringes), blisters, capsules, cartridges, or other packaging. The chamber with components may then be stored at the location of use or transported (756) to the location of use. Depending on the specific implementation, the chambers, holding components, may be refrigerated, frozen, or otherwise maintained in a temperature-controlled environment during transportation and storage.

When ready for use, the components in the chamber may be combined with one or more diluents (758) to yield the desired concentration for final use. For example, the chambers produced at step (754) may, for example, contain concentrated mixture needing dilution or solids needing to be dissolved, for example in a bottle, vial, syringe, or other container. Alternatively, when ready for use, if the components introduced into the chamber are such that no dilution is needed then the contents are not diluted. Alternatively, part or all of the contents of the chamber may be withdrawn and added to another container, a non-limiting example being a bag containing an IV solution. At this stage other materials may be added.

Finally, the resulting composition may be applied (760) for the desired use. As otherwise noted herein, the stabilized RNA products of the present invention may be utilized in a variety of fields such as therapeutics, diagnostics, or agriculture. In addition, the product may be packaged and distributed as a medicament, a therapeutic, vaccine, biostimulant, embedded complex, or implant.

Moreover, in the case of pharmaceutical compositions, the product may be packaged and distributed for administration orally, sublingually, transdermally, ophthalmically, parenterally, subcutaneously, intravenously, intramuscularly, by inhalation, topically, rectally, nasally, buccally, vaginally, or via an implant as otherwise described herein. The RNA product may be used for a variety of applications including treating a disease, preventing a disease, or producing a cellular response as otherwise described herein. The product may be used in in vivo, in vitro, in situ, or ex vivo applications. Such applications may include agricultural applications, agricultural treatment, fertilizer, biostimulant, veterinary applications, animal treatment, pharmaceutical applications, human treatment, therapeutic applications, soil treatment, pesticide, herbicide, bacterial treatment, fungal treatment, antiviral, antibiotic, antifungal, antimicrobial, plant applications, plant treatment, antibody production, protein expression applications, insect treatment, insecticide, vaccine production, therapeutic agent production, implant production, embedded complex production, drug production, medicament production, or combinations thereof. Accordingly, applying the resulting composition for the desired use will vary depending on the nature of the composition and the intended use among other things.

As a non-limiting example, a chamber may be at least partially filled with components comprising one or more RNA substance and one or more RNA stabilizing substance followed by the chamber being prepared for shipping and storage (as a non-limiting example by undergoing steps comprising being packaged or placed in a shipping and storage container) followed by the chamber being transported to the location use, then removed from packaging and prepared for use by adding one or more diluents to the chamber and mixing, and with the appropriate amount of diluted mixture withdrawn from the chamber and administered to a patient as a therapeutic agent.

### EXAMPLES

The following non-limiting examples describe examples of the invention in more detail and in no way are to be construed as limiting the scope thereof.

### Example 1

In Vitro Transcription and Denaturing Agarose Gel Electrophoresis

The RNA was synthesized by in vitro transcription from a linear DNA construct with an upstream T7 RNA Polymerase promoter followed by the coding sequence for gene of interest. In vitro transcription was performed using the HiScribe T7 Quick High Yield RNA Synthesis Kit (New England Biolabs, Ipswich, Mass.; Product #E2050) according to the manufacturer's directions. Briefly, 2.5 μg template DNA was mixed with 25 μL NTP buffer mix and 5 μL T7 RNA polymerase mix. The entire reaction volume was brought to 50 μL with molecular biology grade H₂O and incubated in a thermal cycler at 37° C. for 2 hrs.

Following in vitro transcription, the RNA was purified using a Monarch RNA Cleanup Kit (New England Biolabs, Ipswich, Mass.; Product #T2050) according to the manufacturer's directions. Briefly, 1 spin column was used for each 50 μL reaction. Following binding of the RNA to the spin column, 2 washes of 500 μL were performed and the RNA was eluted with 100-150 μL of molecular biology grade H₂O. The purified RNA was then stored at −80° C.

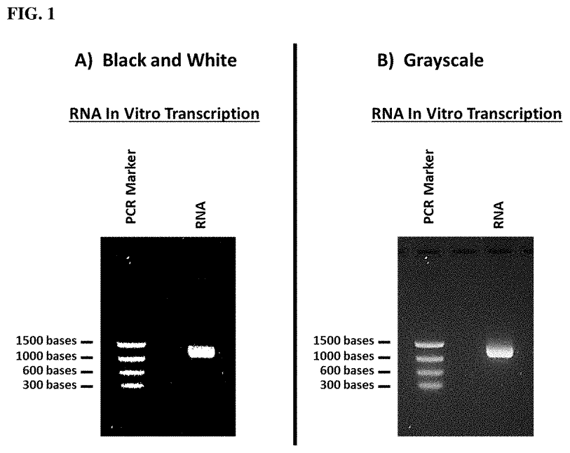

The in vitro transcribed and purified RNA was analyzed by denaturing agarose gel electrophoresis. Briefly, about 5 μg RNA was diluted 1:2 with 2×RNA loading dye (New England Biolabs, Ipswich, Mass.; Product #B0363) and heated to about 70° C. for about 2 minutes to denature the RNA. The final concentration of RNA and loading dye was about: 5 μg RNA, 47.5% formamide, 0.01% SDS, 0.01% bromophenol blue, 0.005% xylene cyanol and 0.5 mM EDTA. The RNA was run on a 1.5% agarose gel in 1×Tris Acetate EDTA (TAE) (Tris 40 mM, Acetic acid 20 mM, EDTA 1 mM, pH 8.0) supplemented with 0.06% sodium hypochlorite (NaClO) to prevent renaturing and degradation of the RNA during electrophoresis. The running buffer also contained 1×TAE supplemented with 0.06% sodium hypochlorite. RNA was visualized using SmartGlow fluorescent nucleic acid prestain (Accuris Instruments, Edison, N.J.; Product #E4500-PS) according to the manufacturer's directions. A double stranded DNA PCR Marker (New England Biolabs, Ipswich, Mass.; Product #N3234) was used to estimate the apparent molecular weight of the RNA during electrophoresis. Denaturing agarose gel electrophoresis was carried out for about 1 hr at 80 v. The in vitro transcribed and purified RNA analyzed by denaturing agarose gel electrophoresis is shown in FIG. 1.

RNA concentration was measured by absorbance of the purified RNA at 260 nm using a Nanodrop ND-1000 (Thermo Fisher Scientific, Waltham, Mass.). Typical RNA concentration following in vitro transcription and purification was about 2-5 mg/mL.

### Example 2

Stability of RNA at Various Temperatures

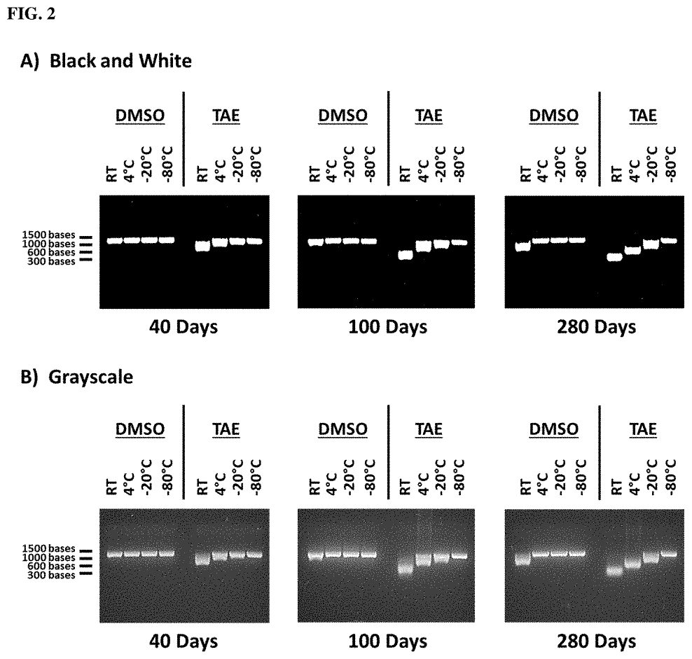

In vitro transcribed RNA was diluted at a ratio of about 1:10 (about 400-500 μg/mL) in either DMSO (Sigma-Aldrich, St. Louis, Mo.; Product #D8418) or 1×Tris Acetate EDTA (TAE) (pH 8) (Bioland Scientific LLC, Paramount, Calif.; Product #TAE01). The final concentration of DMSO was about 90% DMSO. The final concentration of TAE was about Tris 40 mM, Acetic acid 20 mM, EDTA 1 mM, pH 8.0. Following dilution of the RNA in either DMSO or TAE, the samples were then stored at 4 different temperatures: room temperature (RT) (about 20-25° C.), about 4° C., about −20° C., and about −80° C. Samples were then analyzed by denaturing agarose gel electrophoresis as described above at selected timepoints to measure RNA degradation and the stability of the RNA samples stored in either DMSO or TAE. During storage, 10 μL of each sample was analyzed at selected timepoints by agarose gel electrophoresis to measure RNA degradation and the stability of the RNA samples stored at each temperature in either DMSO or TAE. FIG. 2 shows the agarose gel electrophoresis of each RNA sample following storage of the RNA for about 40 days to about 280 days at various temperatures.

The RNA sample stored in DMSO displays increased stability and reduced rate of degradation of the RNA sample as shown by agarose gel electrophoresis. The RNA sample stored in TAE begins to show notable degradation at room temperature after about 40 days as indicated by the smearing of the RNA band, decreased fluorescence intensity, and lower apparent molecular weight compared to the −80° C. RNA sample. While the RNA sample stored in DMSO does not show notable signs of degradation until about 100 days at room temperature. Furthermore, the RNA sample stored in TAE begins to show notable signs of degradation following about 40 days at 4° C. While the RNA sample stored in DMSO does not show notable signs of degradation up to about 280 days at 4° C. In addition, the RNA sample stored in TAE begins to show notable signs of degradation following about 100 days at −20° C. While the RNA sample stored in DMSO does not show notable signs of degradation up to about 280 days at −20° C. Following 100 days, it becomes apparent that the RNA sample stored in DMSO shows comparable and/or better stability at room temperature when compared to the RNA sample stored in TAE at 4° C. or −20° C. Furthermore, RNA stored in DMSO at 4° C. still displays a measurable band of comparable size and fluorescence intensity compared to the −80° C. RNA sample following 280 days, while the RNA stored in TAE shows little to no band of comparable size and fluorescence intensity compared to the −80° C. RNA sample following 280 days at 4° C. or −20° C.

### Example 3

Accelerated RNA Stability Testing at 60° C.

In vitro transcribed RNA was diluted at a ratio of about 1:10 (about 200-300 ug/mL) in different compositions containing either 50 mM sodium acetate (pH 5.2) (Sigma-Aldrich, St. Louis, Mo.; Product #S7899), or a mixture of DMSO and 50 mM sodium acetate (pH 5.2) as follows:
1. 50 mM Na-Acetate (pH 5.2)
2. 70% DMSO+50 mM Na-Acetate (pH 5.2)
3. 60% DMSO+50 mM Na-Acetate (pH 5.2)
4. 50% DMSO+50 mM Na-Acetate (pH 5.2)
5. −80° C.

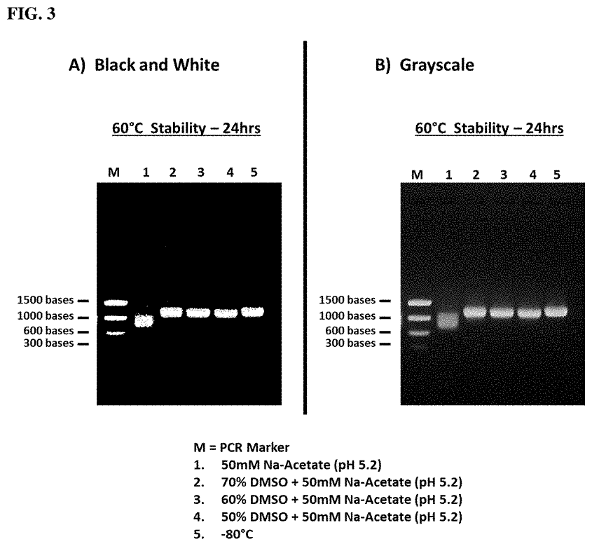

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. FIG. 3 shows the agarose gel following storage of the RNA for about 24 hours at 60° C. Following about 24 hours at 60° C., samples 2-4 comprising varying concentrations of DMSO display significantly less RNA degradation as compared to sample 1 with only 50 mM sodium acetate (pH 5.2) as indicated by the bands of relatively comparable size and fluorescence intensity as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 4

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or a mixture of N-Methyl-2-pyrrolidone (NMP) (Sigma-Aldrich, St. Louis, Mo.; Product #270458) and 50 mM sodium acetate (pH 5.2) as follows:
1. 50 mM Na-Acetate (pH 5.2)
2. 50% NMP+50 mM Na-Acetate (pH 5.2)
3. 40% NMP+50 mM Na-Acetate (pH 5.2)
4. 30% NMP+50 mM Na-Acetate (pH 5.2)
5. −80° C.

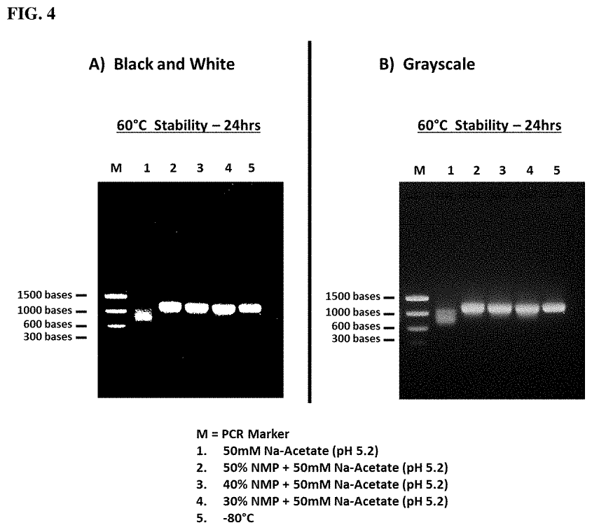

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. FIG. 4 shows the agarose gel following storage of the RNA for about 24 hours at 60° C. Following about 24 hours at 60° C., samples 2-4 comprising varying concentrations of NMP display significantly less RNA degradation as compared to sample 1 with only 50 mM sodium acetate (pH 5.2) as indicated by the bands of relatively comparable size and fluorescence intensity as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 5

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or a mixture of sodium trimetaphosphate (TMP) (Sigma-Aldrich, St. Louis, Mo.; Product #PHR2204) and 50 mM sodium acetate (pH 5.2) as follows:
1. 50 mM Na-Acetate (pH 5.2)
2. 100 mM Na-Trimetaphosphate+50 mM Na-Acetate (pH 5.2)
3. 90 mM Na-Trimetaphosphate+50 mM Na-Acetate (pH 5.2)
4. 80 mM Na-Trimetaphosphate+50 mM Na-Acetate (pH 5.2)
5. −80° C.

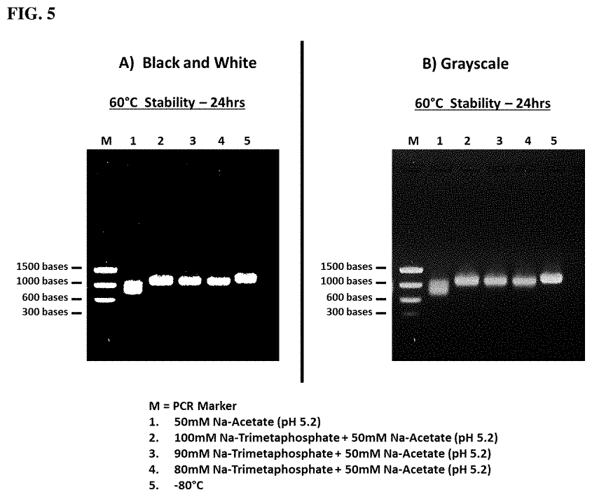

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. FIG. 5 shows the agarose gel following storage of the RNA for about 24 hours at 60° C. Following about 24 hours at 60° C., samples 2-4 comprising varying concentrations of TMP display significantly less RNA degradation as compared to sample 1 with only 50 mM sodium acetate (pH 5.2) as indicated by the bands of relatively comparable size and fluorescence intensity as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 6

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or a mixture of sodium hexametaphosphate (HMP) (Sigma-Aldrich, St. Louis, Mo.; Product #305553) and 50 mM sodium acetate (pH 5.2) as follows:
1. 50 mM Na-Acetate (pH 5.2)
2. 40 mM Na-Hexametaphosphate+50 mM Na-Acetate (pH 5.2)
3. 30 mM Na-Hexametaphosphate+50 mM Na-Acetate (pH 5.2)
4. 20 mM Na-Hexametaphosphate+50 mM Na-Acetate (pH 5.2)
5. −80° C.

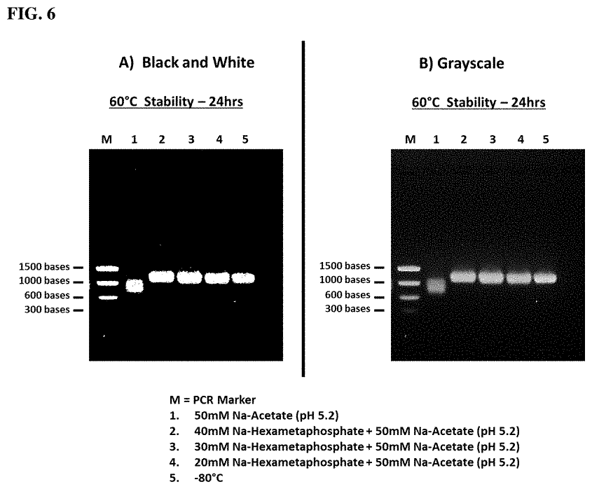

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. FIG. 6 shows the agarose gel following storage of the RNA for about 24 hours at 60° C. Following about 24 hours at 60° C., samples 2-4 comprising varying concentrations of HMP display significantly less RNA degradation as compared to sample 1 with only 50 mM sodium acetate (pH 5.2) as indicated by the bands of relatively comparable size and fluorescence intensity as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 7

Accelerated RNA Stability Testing at 60° C.

In vitro transcribed RNA was diluted at a ratio of about 1:10 (about 200-300 ug/mL) in different compositions containing either 50 mM Tris-HCl (pH 7) (Bioland Scientific LLC, Paramount, Calif.; Product #Tris70), or a mixture of sodium hexametaphosphate (HMP) and 50 mM Tris-HCl (pH 7) as follows:
1. 50 mM Tris-HCl (pH 7)
2. 40 mM Na-Hexametaphosphate+50 mM Tris-HCl (pH 7)
3. 30 mM Na-Hexametaphosphate+50 mM Tris-HCl (pH 7)
4. 20 mM Na-Hexametaphosphate+50 mM Tris-HCl (pH 7)
5. −80° C.

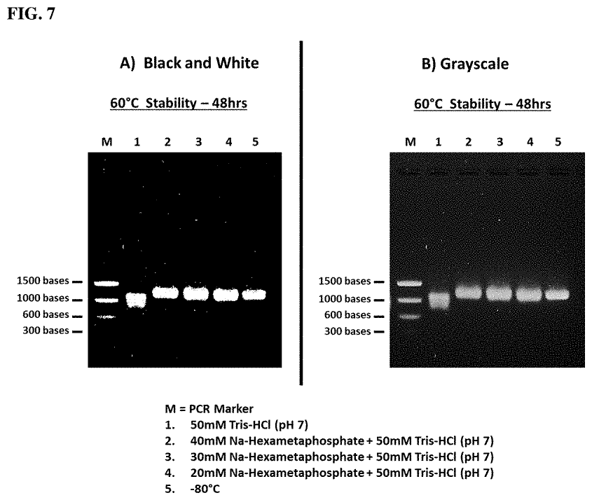

Following dilution of each sample in each respective RNA storage environment, samples were stored in a thermal cycler at 60° C. for about 48 hours. During storage at 60° C. samples were analyzed at selected timepoints by denaturing agarose gel electrophoresis to measure RNA degradation and the stability of the RNA samples in each RNA storage environment. FIG. 7 shows the agarose gel following storage of the RNA for about 48 hours at 60° C. Following about 48 hours at 60° C., samples 2-4 comprising varying concentrations of HMP display significantly less RNA degradation as compared to sample 1 with only 50 mM Tris-HCl (pH 7) as indicated by the bands of relatively comparable size and fluorescence intensity as compared to the −80° C. reference sample. Meanwhile, sample 1 with only 50 mM Tris-HCl (pH 7) displays significant RNA degradation as indicated by the broadening of the RNA band, decreased fluorescence intensity, and lower apparent molecular weight.

### Example 8

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or a mixture of hexylene glycol (Sigma-Aldrich, St. Louis, Mo.; Product #M9671) and 50 mM sodium acetate (pH 5.2) as follows:
1. 50 mM Na-Acetate (pH 5.2)
2. 30% Hexylene Glycol+50 mM Na-Acetate (pH 5.2)
3. 27.5% Hexylene Glycol+50 mM Na-Acetate (pH 5.2)
4. 25% Hexylene Glycol+50 mM Na-Acetate (pH 5.2)
5. 22.5% Hexylene Glycol+50 mM Na-Acetate (pH 5.2)
6. −80° C.

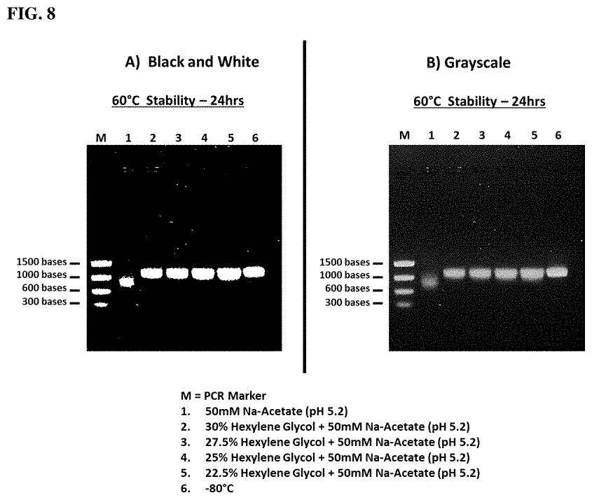

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. Prior to performing gel electrophoresis, the hexylene glycol samples were diluted 1.5× with molecular biology H₂O (10 μL H₂O added to a 20 μL sample) and then incubated with 2.5 mM ˜8 kDa poly(acrylic acid, sodium salt) (PAA) (Sigma-Aldrich, St. Louis, Mo.; Product #416029) for 1 hour at room temperature to improve analysis by gel electrophoresis. FIG. 8 shows the agarose gel following storage of the RNA for about 24 hours at 60° C. Following about 24 hours at 60° C., samples 2-5 comprising varying concentrations of hexylene glycol display significantly less RNA degradation as compared to sample 1 with only 50 mM sodium acetate (pH 5.2) as indicated by the bands of relatively comparable size and fluorescence intensity as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 9

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or a mixture of glycerol phosphate disodium salt (Sigma-Aldrich, St. Louis, Mo.; Product #G6501) and 50 mM sodium acetate (pH 5.2) as follows:
1. 50 mM Na-Acetate (pH 5.2)
2. 200 mM Na-Glycerol Phosphate+50 mM Na-Acetate (pH 5.2)
3. 150 mM Na-Glycerol Phosphate+50 mM Na-Acetate (pH 5.2)
4. 100 mM Na-Glycerol Phosphate+50 mM Na-Acetate (pH 5.2)
5. 50 mM Na-Glycerol Phosphate+50 mM Na-Acetate (pH 5.2)
6. −80° C.

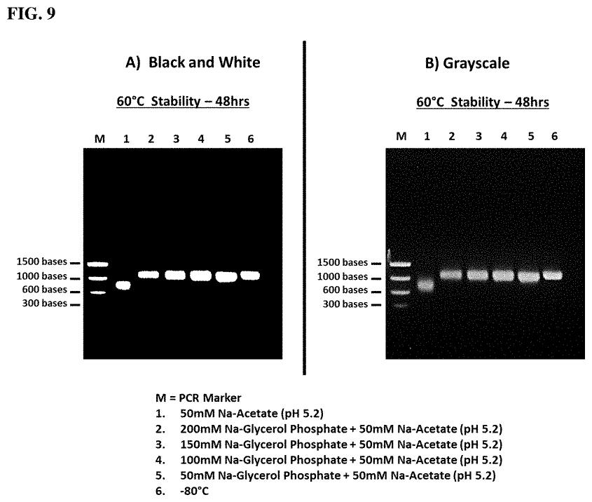

Following dilution of each sample in each respective RNA storage environment, samples were stored in a thermal cycler at 60° C. for about 48 hours. During storage at 60° C. samples were analyzed at selected timepoints by denaturing agarose gel electrophoresis to measure RNA degradation and the stability of the RNA samples in each RNA storage environment. FIG. 9 shows the agarose gel following storage of the RNA for about 48 hours at 60° C. Following about 48 hours at 60° C., samples 2-5 comprising varying concentrations of glycerol phosphate display significantly less RNA degradation as compared to sample 1 with only 50 mM sodium acetate (pH 5.2) as indicated by the bands of relatively comparable size and fluorescence intensity as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 10

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or a mixture of 1-butyl-1-methylpyrrolidinium bromide (Sigma-Aldrich, St. Louis, Mo.; Product #04275), benzyltriethylammonium chloride (Sigma-Aldrich, St. Louis, Mo.; Product #146552), or N,N-dimethylphenethylamine (Sigma-Aldrich, St. Louis, Mo.; Product #523801) and 50 mM sodium acetate (pH 5.2) as follows:
1. 50 mM Na-Acetate (pH 5.2)
2. 750 mM 1-Buty-1-Methylpyrrolidinium-Br+50 mM Na-Acetate (pH 5.2)
3. 500 mM Benzyltriethylammonium-Cl+50 mM Na-Acetate (pH 5.2)
4. 100 mM N,N-Dimethylphenethylamine+50 mM Na-Acetate (pH 5.2)
5. −80° C.

The N,N-dimethylphenethylamine sample dilution was made fresh from the stock solution immediately prior to mixing with RNA. Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. Prior to performing gel electrophoresis, the 1-butyl-1-methylpyrrolidinium and benzyltriethylammonium samples were incubated with 2.5 mM ˜8 kDa PAA sodium salt for about 1 hour at room temperature (about 20-25° C.) to improve analysis by gel electrophoresis. FIG. shows the agarose gel following storage of the RNA for about 24 hours at 60° C. Following about 24 hours at 60° C., samples 2-4 comprising either 1-butyl-1-methylpyrrolidinium, benzyltriethylammonium, or N,N-dimethylphenethylamine display significantly less RNA degradation as compared to sample 1 with only 50 mM sodium acetate (pH 5.2) as indicated by the bands of relatively comparable size and fluorescence intensity of samples 2-4 as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 11

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or a mixture of sodium benzoate (Sigma-Aldrich, St. Louis, Mo.; Product #109169) and 50 mM sodium acetate (pH 5.2) as follows:
1. 50 mM Na-Acetate (pH 5.2)
2. 200 mM Na-Benzoate+50 mM Na-Acetate (pH 5.2)
3. 150 mM Na-Benzoate+50 mM Na-Acetate (pH 5.2)
4. 100 mM Na-Benzoate+50 mM Na-Acetate (pH 5.2)
5. −80° C.

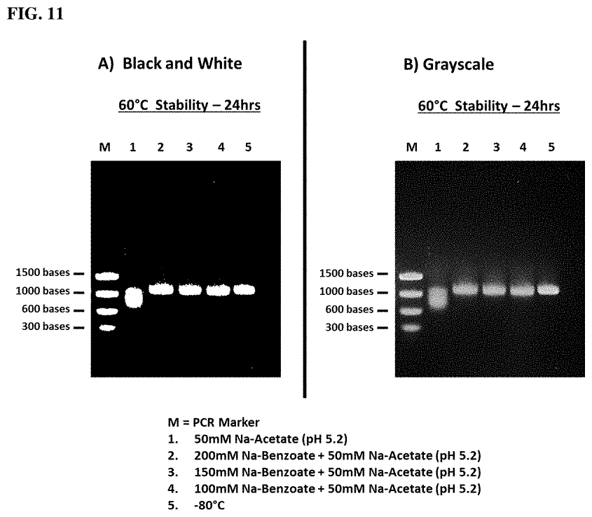

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. FIG. 11 shows the agarose gel following storage of the RNA for about 24 hours at 60° C. Following about 24 hours at 60° C., samples 2-4 comprising varying concentrations of benzoate display significantly less RNA degradation as compared to sample 1 with only 50 mM sodium acetate (pH 5.2) as indicated by the bands of relatively comparable size and fluorescence intensity as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 12

Accelerated RNA Stability Testing at 60° C.

In vitro transcribed RNA was diluted at a ratio of about 1:10 (about 200-300 ug/mL) in different compositions containing either 50 mM Tris-HCl (pH 7), or a mixture of sodium benzoate with 1M trimethylglycine (TMG) (Sigma-Aldrich, St. Louis, Mo.; Product #B0300) and 50 mM Tris-HCl (pH 7) as follows:
1. 50 mM Tris-HCl (pH 7)
2. 300 mM Na-Benzoate+1M TMG+50 mM Tris-HCl (pH 7)
3. 250 mM Na-Benzoate+1M TMG+50 mM Tris-HCl (pH 7)
4. 200 mM Na-Benzoate+1M TMG+50 mM Tris-HCl (pH 7)
5. −80° C.

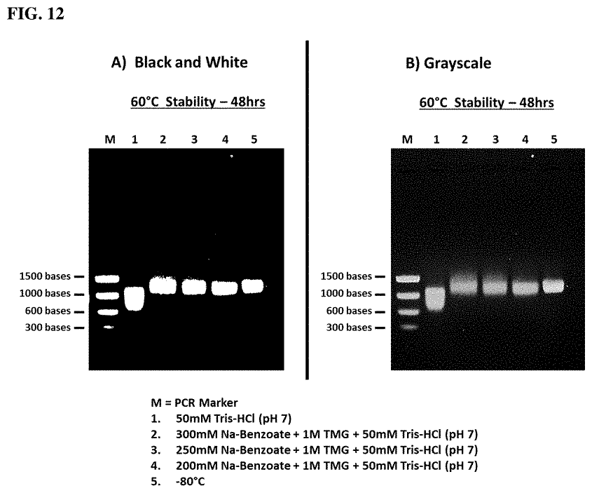

Following dilution of each sample in each respective RNA storage environment, samples were stored in a thermal cycler at 60° C. for about 48 hours. During storage at 60° C. samples were analyzed at selected timepoints by denaturing agarose gel electrophoresis to measure RNA degradation and the stability of the RNA samples in each RNA storage environment. FIG. 12 shows the agarose gel following storage of the RNA for about 48 hours at 60° C. Following about 48 hours at 60° C., samples 2-4 comprising varying concentrations of benzoate with 1M TMG display significantly less RNA degradation as compared to sample 1 with only 50 mM Tris-HCl (pH 7) as indicated by the bands of relatively comparable size and fluorescence intensity of samples 2-4 as compared to the −80° C. reference sample. Meanwhile, sample 1 with only 50 mM Tris-HCl (pH 7) displays significant RNA degradation as indicated by the broadening of the RNA band, decreased fluorescence intensity, and lower apparent molecular weight.

### Example 13

Accelerated RNA Stability Testing at 60° C.

In vitro transcribed RNA was diluted at a ratio of about 1:10 (about 200-300 ug/mL) in different compositions containing either 50% DMSO with 50 mM Tris-HCl (pH 7), or a mixture of sodium benzoate with 50% DMSO and 50 mM Tris-HCl (pH 7) as follows:
1. 50% DMSO+50 mM Tris-HCl (pH 7)
2. 250 mM Na-Benzoate+50% DMSO+50 mM Tris-HCl (pH 7)
3. 200 mM Na-Benzoate+50% DMSO+50 mM Tris-HCl (pH 7)
4. 150 mM Na-Benzoate+50% DMSO+50 mM Tris-HCl (pH 7)
5. 100 mM Na-Benzoate+50% DMSO+50 mM Tris-HCl (pH 7)
6. −80° C.

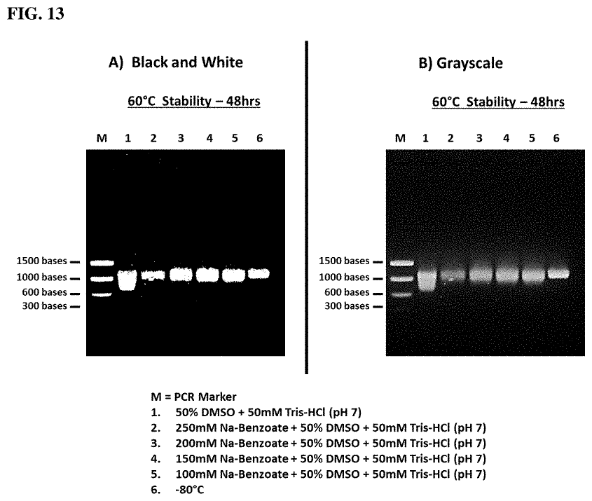

Following dilution of each sample in each respective RNA storage environment, samples were stored in a thermal cycler at 60° C. for about 48 hours. During storage at 60° C. samples were analyzed at selected timepoints by denaturing agarose gel electrophoresis to measure RNA degradation and the stability of the RNA samples in each RNA storage environment. FIG. 13 shows the agarose gel following storage of the RNA for about 48 hours at 60° C. Following about 48 hours at 60° C., samples 2-5 comprising varying concentrations of benzoate with 50% DMSO display significantly less RNA degradation as compared to sample 1 with only 50% DMSO and 50 mM Tris-HCl (pH 7) as indicated by the bands of relatively comparable size and fluorescence intensity of samples 2-5 as compared to the −80° C. reference sample. Meanwhile, sample 1 with only 50% DMSO and 50 mM Tris-HCl (pH 7) displays significant RNA degradation as indicated by the broadening of the RNA band, decreased fluorescence intensity, and lower apparent molecular weight.

### Example 14

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or varying concentrations of trimethyloctylammonium bromide (Sigma-Aldrich, St. Louis, Mo.; Product #75091) and 50 mM sodium acetate (pH 5.2) as follows:
1. 50 mM Na-Acetate (pH 5.2)
2. 70 mM Trimethyloctylammonium-Br+50 mM Na-Acetate (pH 5.2)
3. 60 mM Trimethyloctylammonium-Br+50 mM Na-Acetate (pH 5.2)
4. 50 mM Trimethyloctylammonium-Br+50 mM Na-Acetate (pH 5.2)
5. 40 mM Trimethyloctylammonium-Br+50 mM Na-Acetate (pH 5.2)
6. −80° C.

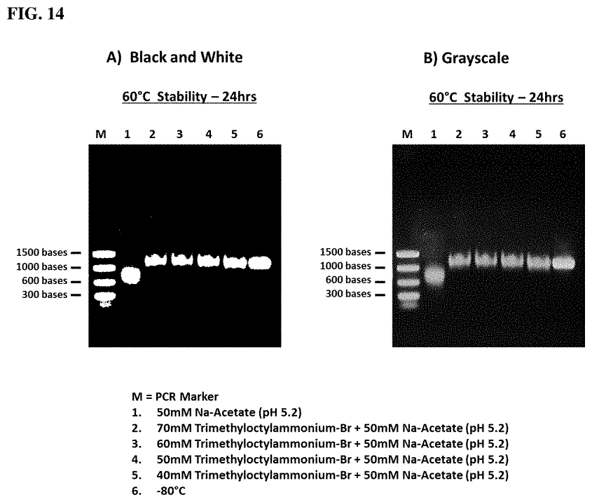

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. Prior to performing gel electrophoresis, the trimethyloctylammonium samples were diluted 1.5× with molecular biology H₂O (10 μL H₂O added to a 20 μL sample) and then incubated with 2.5 mM ˜8 kDa PAA sodium salt for about 1 hour at room temperature (about 20-25° C.) to improve analysis by gel electrophoresis. FIG. 14 shows the agarose gel following storage of the RNA for about 24 hours at 60° C. Following about 24 hours at 60° C., samples 2-5 comprising varying concentrations of trimethyloctylammonium display significantly less RNA degradation as compared to sample 1 with only 50 mM sodium acetate (pH 5.2) as indicated by the bands of relatively comparable size and fluorescence intensity of samples 2-5 as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 15

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or a mixture of quinolinic acid (Cayman Chemical, Ann Arbor, Mich.; Product #14941), nicotinamide N-oxide (Cayman Chemical, Ann Arbor, Mich.; Product #28441), nicotinic acid (Sigma-Aldrich, St. Louis, Mo.; Product #N4126), or 1-methylnicotinamide chloride (Cayman Chemical, Ann Arbor, Mich.; Product #16604) and 50 mM sodium acetate (pH 5.2) as follows:
1. 50 mM Na-Acetate (pH 5.2)
2. 100 mM Na-Quinolinic Acid (pH 7)+50 mM Na-Acetate (pH 5.2)
3. 100 mM Nicotinamide N-Oxide+50 mM Na-Acetate (pH 5.2)
4. 100 mM Na-Nicotinic Acid (pH 7)+50 mM Na-Acetate (pH 5.2)
5. 1M 1-Methylnicotinamide-Cl+50 mM Na-Acetate (pH 5.2)
6. −80° C.

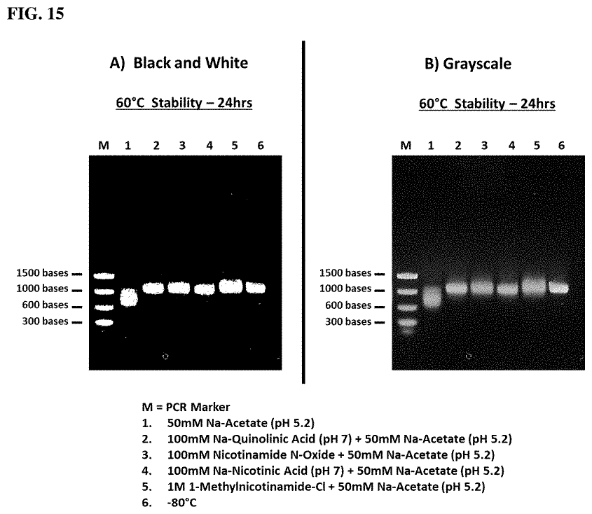

The quinolinic acid and nicotinic acid stocks were adjusted to a pH of about 7 using NaOH. The nicotinamide N-oxide stock pH was adjusted using NaOH to facilitate dissolving in H₂O and then subsequently adjusted to a pH of about 7 using HCl once dissolved. Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. Prior to performing gel electrophoresis, the nicotinamide N-oxide and 1-methylnicotinamide samples were incubated with 1 mM ˜8 kDa PAA sodium salt about 1 hour at room temperature (about 20-25° C.) to improve analysis by gel electrophoresis. FIG. 15 shows the agarose gel following storage of the RNA for about 24 hours at 60° C. Following about 24 hours at 60° C., samples 2-5 comprising either quinolinic acid, nicotinamide N-oxide, nicotinic acid, or 1-methylnicotinamide display significantly less RNA degradation as compared to sample 1 with only 50 mM sodium acetate (pH 5.2) as indicated by the bands of relatively comparable size and fluorescence intensity of samples 2-5 as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 16

Accelerated RNA Stability Testing at 60° C.

In vitro transcribed RNA was diluted at a ratio of about 1:10 (about 200-300 ug/mL) in different compositions containing either 5 mM sodium phosphate (pH 8) (Sigma-Aldrich, St. Louis, Mo.; Product #94046 & 74092) and 10 mM sodium acetate (pH 7) (Sigma-Aldrich, St. Louis, Mo.; Product #S2404), or a mixture of ectoine (Sigma-Aldrich, St. Louis, Mo.; Product #81619), L-proline (Sigma-Aldrich, St. Louis, Mo.; Product #81709), glycine (Sigma-Aldrich, St. Louis, Mo.; Product #50046), or taurine (Sigma-Aldrich, St. Louis, Mo.; Product #T0625), and 5 mM sodium phosphate (pH 8) and 10 mM sodium acetate (pH 7) as follows:
1. 5 mM Na-Phosphate (pH 8)+10 mM Na-Acetate (pH 7)
2. 2M Ectoine+5 mM Na-Phosphate (pH 8)+10 mM Na-Acetate (pH 7)
3. 500 mM Proline+5 mM Na-Phosphate (pH 8)+10 mM Na-Acetate (pH 7)
4. 500 mM Glycine+5 mM Na-Phosphate (pH 8)+10 mM Na-Acetate (pH 7)
5. 250 mM Taurine+5 mM Na-Phosphate (pH 8)+10 mM Na-Acetate (pH 7)
6. −80° C.

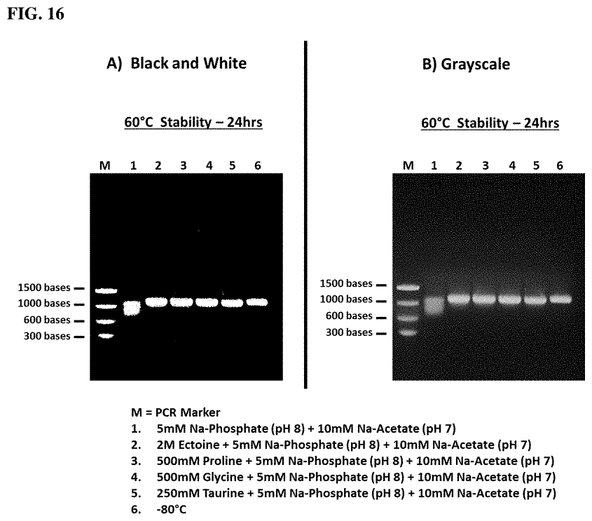

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. FIG. 16 shows the agarose gel following storage of the RNA for about 24 hours at 60° C. Following about 24 hours at 60° C., samples 2-5 comprising either ectoine, proline, glycine, or taurine display significantly less RNA degradation as compared to sample 1 with only 5 mM sodium phosphate (pH 8) and 10 mM sodium acetate (pH 7) as indicated by the bands of relatively comparable size and fluorescence intensity of samples 2-5 as compared to the −80° C. reference sample. Meanwhile, sample 1 with only 5 mM sodium phosphate (pH 8) and 10 mM sodium acetate (pH 7) displays significant RNA degradation as indicated by the broadening of the RNA band, decreased fluorescence intensity, and lower apparent molecular weight.

### Example 17

Accelerated RNA Stability Testing at 60° C.

In vitro transcribed RNA was diluted at a ratio of about 1:10 (about 200-300 ug/mL) in different compositions containing either 60% DMSO with 50 mM sodium acetate (pH 7), or a mixture of ectoine, L-proline, glycine, or taurine, and 60% DMSO with 50 mM sodium acetate (pH 7) as follows:
1. 60% DMSO+50 mM Na-Acetate (pH 7)
2. 1M Ectoine+60% DMSO+50 mM Na-Acetate (pH 7)
3. 300 mM Proline+60% DMSO+50 mM Na-Acetate (pH 7)
4. 300 mM Glycine+60% DMSO+50 mM Na-Acetate (pH 7)
5. 125 mM Taurine+60% DMSO+50 mM Na-Acetate (pH 7)
6. −80° C.

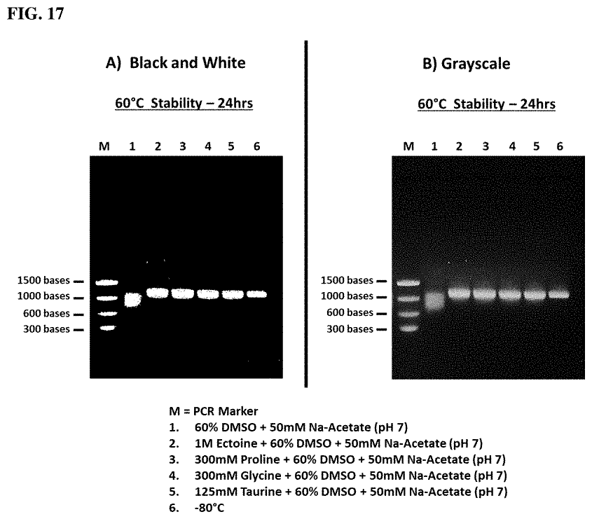

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. FIG. 17 shows the agarose gel following storage of the RNA for about 24 hours at 60° C. Following about 24 hours at 60° C., samples 2-5 comprising either ectoine, proline, glycine, or taurine display significantly less RNA degradation as compared to sample 1 with only 60% DMSO and 50 mM sodium acetate (pH 7) as indicated by the bands of relatively comparable size and fluorescence intensity of samples 2-5 as compared to the −80° C. reference sample. Meanwhile, sample 1 with only 60% DMSO and 50 mM sodium acetate (pH 7) displays significant RNA degradation as indicated by the broadening of the RNA band, decreased fluorescence intensity, and lower apparent molecular weight.

### Example 18

Accelerated RNA Stability Testing at 60° C.

In vitro transcribed RNA was diluted at a ratio of about 1:10 (about 200-300 ug/mL) in different compositions containing either TAE (Tris 40 mM, Acetic acid 20 mM, EDTA 1 mM) (pH 8), or a mixture of dimethylsulfoniopropionate (DMSP) (Sigma-Aldrich, St. Louis, Mo.; Product #80828) with TAE (pH 8) as follows:
1. TAE (pH 8)
2. 700 mM DMSP+TAE (pH 8)
3. 600 mM DMSP+TAE (pH 8)
4. 500 mM DMSP+TAE (pH 8)
5. 400 mM DMSP+TAE (pH 8)
6. −80° C.

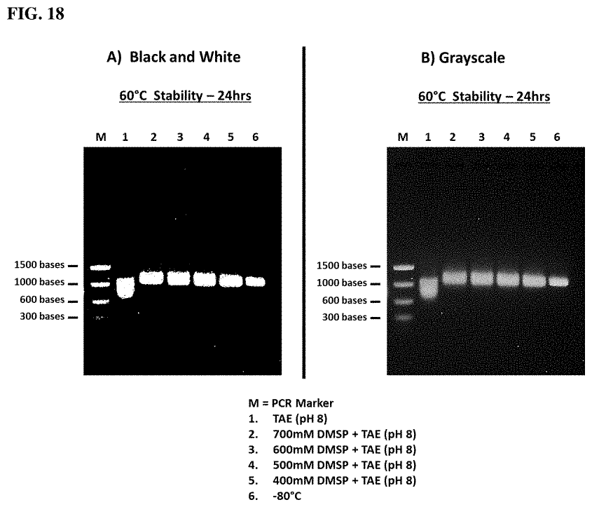

The DMSP stock was adjusted to a pH of about 7 using NaOH. Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. Prior to performing gel electrophoresis, the DMSP samples were incubated with 1 mM ˜8 kDa PAA sodium salt about 1 hour at room temperature (about 20-25° C.) to improve analysis by gel electrophoresis. FIG. 18 shows the agarose gel following storage of the RNA for about 24 hours at 60° C. Following about 24 hours at 60° C., samples 2-5 comprising varying concentrations of DMSP display significantly less RNA degradation as compared to sample 1 with only TAE (pH 8) as indicated by the bands of relatively comparable size and fluorescence intensity of samples 2-5 as compared to the −80° C. reference sample. Meanwhile, sample 1 with only TAE (pH 8) displays significant RNA degradation as indicated by the broadening of the RNA band, decreased fluorescence intensity, and lower apparent molecular weight.

### Example 19

Accelerated RNA Stability Testing at 60° C.

In vitro transcribed RNA was diluted at a ratio of about 1:10 (about 200-300 ug/mL) in different compositions containing either 50% DMSO with 50 mM sodium acetate (pH 7), or a mixture of DMSP and 50% DMSO with 50 mM sodium acetate (pH 7) as follows:
1. 50% DMSO+50 mM Na-Acetate (pH 7)
2. 250 mM DMSP+50% DMSO+50 mM Na-Acetate (pH 7)
3. 200 mM DMSP+50% DMSO+50 mM Na-Acetate (pH 7)
4. 150 mM DMSP+50% DMSO+50 mM Na-Acetate (pH 7)
5. 100 mM DMSP+50% DMSO+50 mM Na-Acetate (pH 7)
6. −80° C.

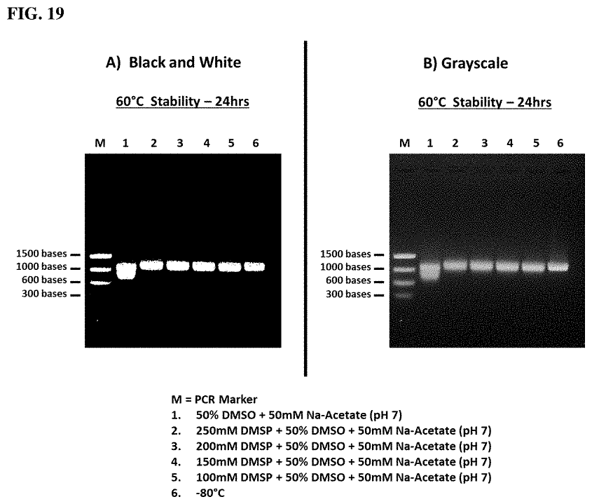

The DMSP stock was adjusted to a pH of about 7 using NaOH. Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. Prior to performing gel electrophoresis, the DMSP samples were incubated with 1 mM ˜8 kDa PAA sodium salt for about 1 hour at room temperature (about 20-25° C.) to improve analysis by gel electrophoresis. FIG. 19 shows the agarose gel following storage of the RNA for about 24 hours at 60° C. Following about 24 hours at 60° C., samples 2-5 comprising varying concentrations of DMSP display significantly less RNA degradation as compared to sample 1 with only 50% DMSO and 50 mM sodium acetate (pH 7) as indicated by the bands of relatively comparable size and fluorescence intensity of samples 2-5 as compared to the −80° C. reference sample. Meanwhile, sample 1 with only 50% DMSO and 50 mM sodium acetate (pH 7) displays significant RNA degradation as indicated by the broadening of the RNA band, decreased fluorescence intensity, and lower apparent molecular weight.

### Example 20

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or a mixture of choline chloride (Sigma-Aldrich, St. Louis, Mo.; Product #26978) and 50 mM sodium acetate (pH 5.2) as follows:
1. 50 mM Na-Acetate (pH 5.2)
2. 1M Choline-Cl+50 mM Na-Acetate (pH 5.2)
3. 900 mM Choline-Cl+50 mM Na-Acetate (pH 5.2)
4. 8000 mM Choline-Cl+50 mM Na-Acetate (pH 5.2)
5. 700 mM Choline-Cl+50 mM Na-Acetate (pH 5.2)
6. −80° C.

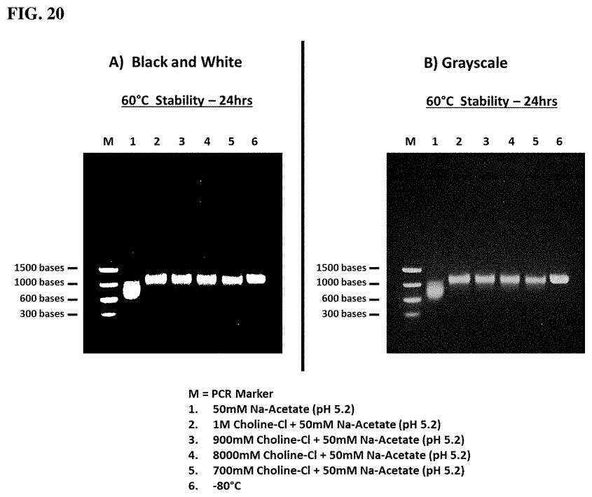

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. FIG. 20 shows the agarose gel following storage of the RNA for about 24 hours at 60° C. Following about 24 hours at 60° C., samples 2-5 comprising varying concentrations of choline display significantly less RNA degradation as compared to sample 1 with only 50 mM sodium acetate (pH 5.2) as indicated by the bands of relatively comparable size and fluorescence intensity of samples 2-5 as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 21

Accelerated RNA Stability Testing at 70° C.

In vitro transcribed RNA was diluted at a ratio of about 1:10 (about 200-300 ug/mL) in different compositions containing either 50 mM Tris-HCl (pH 7), or a mixture of choline chloride and 50 mM Tris-HCl (pH 7) as follows:
1. 50 mM Tris-HCl (pH 7)
2. 1M Choline-Cl+50 mM Tris-HCl (pH 7)
3. 900 mM Choline-Cl+50 mM Tris-HCl (pH 7)
4. 800 mM Choline-Cl+50 mM Tris-HCl (pH 7)
5. 700 mM Choline-Cl+50 mM Tris-HCl (pH 7)
6. −80° C.

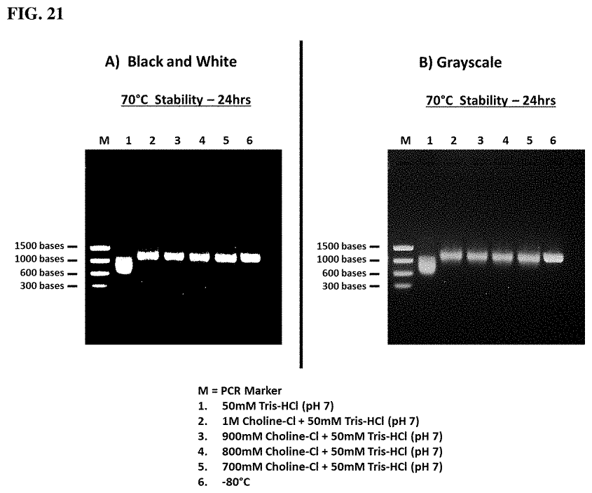

Following dilution of each sample in each respective RNA storage environment, samples were stored in a thermal cycler at 70° C. for about 24 hours. During storage at 70° C. samples were analyzed at selected timepoints by denaturing agarose gel electrophoresis to measure RNA degradation and the stability of the RNA samples in each RNA storage environment. FIG. 21 shows the agarose gel following storage of the RNA for about 24 hours at 70° C. Following about 24 hours at 70° C., samples 2-5 comprising varying concentrations of choline display significantly less RNA degradation as compared to sample 1 with only 50 mM Tris-HCl (pH 7) as indicated by the bands of relatively comparable size and fluorescence intensity of samples 2-5 as compared to the −80° C. reference sample. Meanwhile, sample 1 with only 50 mM Tris-HCl (pH 7) displays significant RNA degradation as indicated by the broadening of the RNA band, decreased fluorescence intensity, and lower apparent molecular weight.

### Example 22

Accelerated RNA Stability Testing at 60° C.

In vitro transcribed RNA was diluted at a ratio of about 1:10 (about 200-300 ug/mL) in different compositions containing either 50% DMSO with 50 mM Tris-HCl (pH 7), or a mixture of choline chloride and 50% DMSO with 50 mM Tris-HCl (pH 7) as follows:
1. 50% DMSO+50 mM Tris-HCl (pH 7)
2. 700 mM Choline-Cl+50% DMSO+50 mM Tris-HCl (pH 7)
3. 600 mM Choline-Cl+50% DMSO+50 mM Tris-HCl (pH 7)
4. 500 mM Choline-Cl+50% DMSO+50 mM Tris-HCl (pH 7)
5. 400 mM Choline-Cl+50% DMSO+50 mM Tris-HCl (pH 7)
6. −80° C.

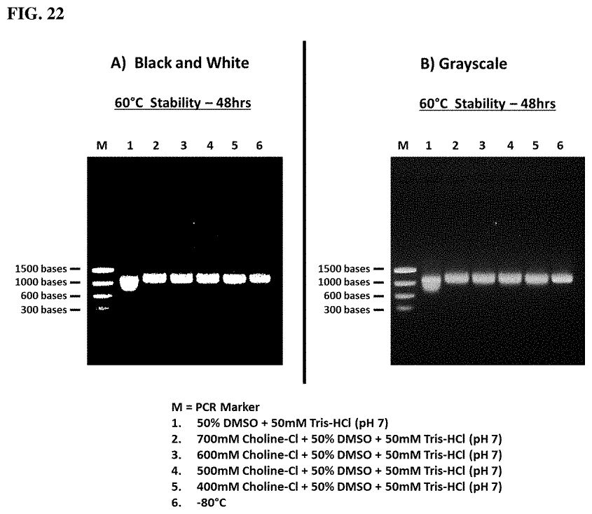

Following dilution of each sample in each respective RNA storage environment, samples were stored in a thermal cycler at 60° C. for about 48 hours. During storage at 60° C. samples were analyzed at selected timepoints by denaturing agarose gel electrophoresis to measure RNA degradation and the stability of the RNA samples in each RNA storage environment. FIG. 22 shows the agarose gel following storage of the RNA for about 48 hours at 60° C. Following about 48 hours at 60° C., samples 2-5 comprising varying concentrations of choline display significantly less RNA degradation as compared to sample 1 with only 50% DMSO and 50 mM Tris-HCl (pH 7) as indicated by the bands of relatively comparable size and fluorescence intensity of samples 2-5 as compared to the −80° C. reference sample. Meanwhile, sample 1 with only 50% DMSO and 50 mM Tris-HCl (pH 7) displays significant RNA degradation as indicated by the broadening of the RNA band, decreased fluorescence intensity, and lower apparent molecular weight.

### Example 23

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or a mixture of acetylcholine chloride (Sigma-Aldrich, St. Louis, Mo.; Product #A2661) and 50 mM sodium acetate (pH 5.2) as follows:
1. 50 mM Na-Acetate (pH 5.2)
2. 1M Acetylcholine-Cl+50 mM Na-Acetate (pH 5.2)
3. 900 mM Acetylcholine-Cl+50 mM Na-Acetate (pH 5.2)
4. 800 mM Acetylcholine-Cl+50 mM Na-Acetate (pH 5.2)
5. 700 mM Acetylcholine-Cl+50 mM Na-Acetate (pH 5.2)
6. −80° C.

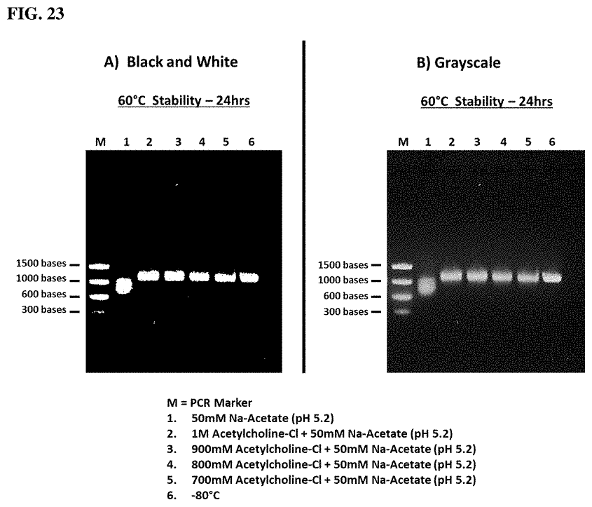

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. FIG. 23 shows the agarose gel following storage of the RNA for about 24 hours at 60° C. Following about 24 hours at 60° C., samples 2-5 comprising varying concentrations of acetylcholine display significantly less RNA degradation as compared to sample 1 with only 50 mM sodium acetate (pH 5.2) as indicated by the bands of relatively comparable size and fluorescence intensity of samples 2-5 as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 24

Accelerated RNA Stability Testing at 60° C.

In vitro transcribed RNA was diluted at a ratio of about 1:10 (about 200-300 ug/mL) in different compositions containing either 5 mM sodium phosphate (pH 8) and 10 mM sodium acetate (pH 7), or a mixture of acetylcholine chloride and 5 mM sodium phosphate (pH 8) and 10 mM sodium acetate (pH 7) as follows:
1. 5 mM Na-Phosphate (pH 8)+10 mM Na-Acetate (pH 7)
2. 1M Acetylcholine-Cl+5 mM Na-Phosphate (pH 8)+10 mM Na-Acetate (pH 7)
3. 500 mM Acetylcholine-Cl+5 mM Na-Phosphate (pH 8)+10 mM Na-Acetate (pH 7)
4. 250 mM Acetylcholine-Cl+5 mM Na-Phosphate (pH 8)+10 mM Na-Acetate (pH 7)
5. 100 mM Acetylcholine-Cl+5 mM Na-Phosphate (pH 8)+10 mM Na-Acetate (pH 7)
6. −80° C.

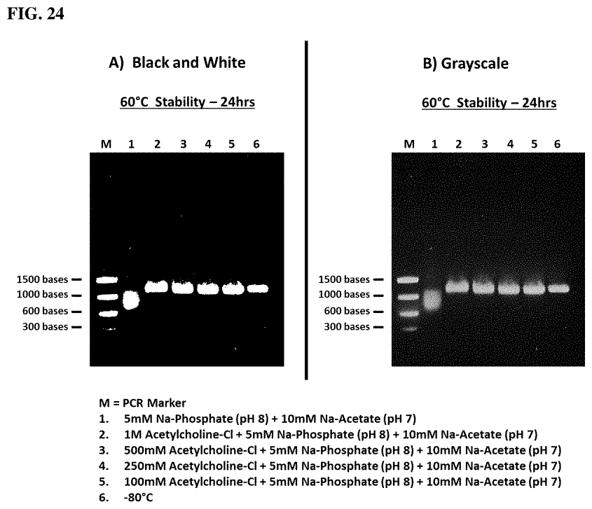

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. FIG. 24 shows the agarose gel following storage of the RNA for about 24 hours at 60° C. Following about 24 hours at 60° C., samples 2-5 comprising varying concentrations of acetylcholine display significantly less RNA degradation as compared to sample 1 with only 5 mM sodium phosphate (pH 8) and 10 mM sodium acetate (pH 7) as indicated by the bands of relatively comparable size and fluorescence intensity of samples 2-5 as compared to the −80° C. reference sample. Meanwhile, sample 1 with only 5 mM sodium phosphate (pH 8) and 10 mM sodium acetate (pH 7) displays significant RNA degradation as indicated by the broadening of the RNA band, decreased fluorescence intensity, and lower apparent molecular weight.

### Example 25

Accelerated RNA Stability Testing at 60° C.

In vitro transcribed RNA was diluted at a ratio of about 1:10 (about 200-300 ug/mL) in different compositions containing either 50% DMSO with 50 mM sodium acetate (pH 7), or a mixture of acetylcholine chloride and 50% DMSO with 50 mM sodium acetate (pH 7) as follows:
1. 50% DMSO+50 mM Na-Acetate (pH 7)
2. 700 mM Acetylcholine-Cl+50% DMSO+50 mM Na-Acetate (pH 7)
3. 600 mM Acetylcholine-Cl+50% DMSO+50 mM Na-Acetate (pH 7)
4. 500 mM Acetylcholine-Cl+50% DMSO+50 mM Na-Acetate (pH 7)
5. 400 mM Acetylcholine-Cl+50% DMSO+50 mM Na-Acetate (pH 7)
6. −80° C.

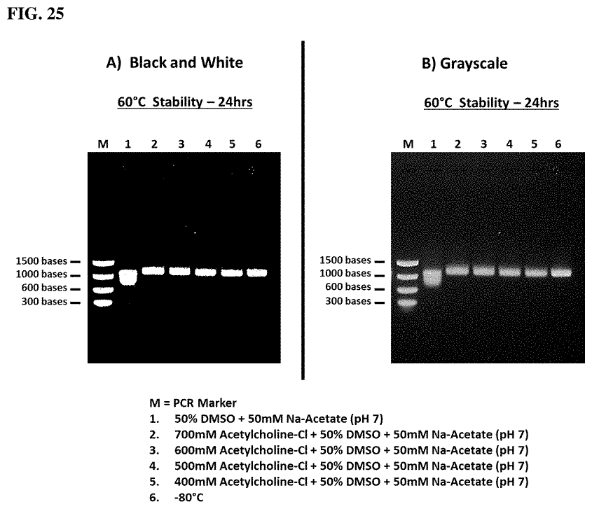

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. FIG. 25 shows the agarose gel following storage of the RNA for about 24 hours at 60° C. Following about 24 hours at 60° C., samples 2-5 comprising varying concentrations of acetylcholine display significantly less RNA degradation as compared to sample 1 with only 50% DMSO and 50 mM sodium acetate (pH 7) as indicated by the bands of relatively comparable size and fluorescence intensity of samples 2-5 as compared to the −80° C. reference sample. Meanwhile, sample 1 with only 50% DMSO and 50 mM sodium acetate (pH 7) displays significant RNA degradation as indicated by the broadening of the RNA band, decreased fluorescence intensity, and lower apparent molecular weight.

### Example 26

Accelerated RNA Stability Testing at 60° C.

In vitro transcribed RNA was diluted at a ratio of about 1:10 (about 200-300 ug/mL) in different compositions containing either 5 mM sodium phosphate (pH 8) and 10 mM sodium acetate (pH 7), or a mixture of TMG (Sigma-Aldrich, St. Louis, Mo.; Product #B0300), NDSB-195 (Sigma-Aldrich, St. Louis, Mo.; Product #D0195), NDSB-221 (Hampton Research, Aliso Viejo, Calif.; Product #HR2-791), or L-carnitine (Cayman Chemical, Ann Arbor, Mich.; Product #21489), and 5 mM sodium phosphate (pH 8) and 10 mM sodium acetate (pH 7) as follows:
1. 5 mM Na-Phosphate (pH 8)+10 mM Na-Acetate (pH 7)
2. 3TMG+5 mM Na-Phosphate (pH 8)+10 mM Na-Acetate (pH 7)
3. 1.5M NDSB-195+5 mM Na-Phosphate (pH 8)+10 mM Na-Acetate (pH 7)
4. 2M NDSB-221+5 mM Na-Phosphate (pH 8)+10 mM Na-Acetate (pH 7)
5. 2M Carnitine+5 mM Na-Phosphate (pH 8)+10 mM Na-Acetate (pH 7)
6. −80° C.

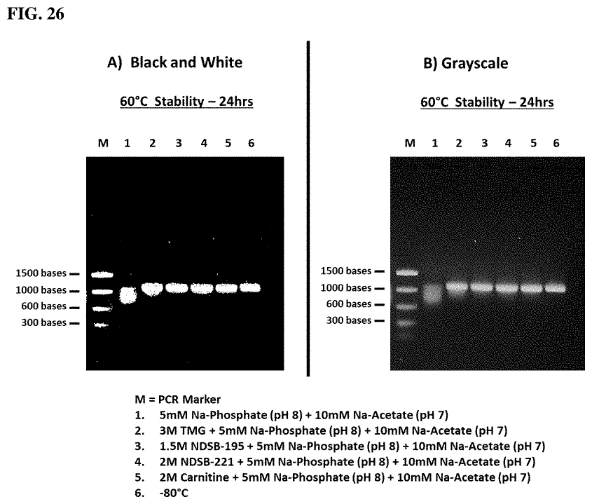

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. FIG. 26 shows the agarose gel following storage of the RNA for about 24 hours at 60° C. Following about 24 hours at 60° C., samples 2-5 comprising either TMG, NDSB-195, NDSB-221, or carnitine display significantly less RNA degradation as compared to sample 1 with only 5 mM sodium phosphate (pH 8) and 10 mM sodium acetate (pH 7) as indicated by the bands of relatively comparable size and fluorescence intensity of samples 2-5 as compared to the −80° C. reference sample. Meanwhile, sample 1 with only 5 mM sodium phosphate (pH 8) and 10 mM sodium acetate (pH 7) displays significant RNA degradation as indicated by the broadening of the RNA band, decreased fluorescence intensity, and lower apparent molecular weight.

### Example 27

Accelerated RNA Stability Testing at 60° C.

In vitro transcribed RNA was diluted at a ratio of about 1:10 (about 200-300 ug/mL) in different compositions containing either 60% DMSO with 50 mM sodium acetate (pH 7), or a mixture of TMG, NDSB-195, NDSB-201 (Sigma-Aldrich, St. Louis, Mo.; Product #82804), NDSB-221, L-carnitine, stachydrine (Cayman Chemical, Ann Arbor, Mich.; Product #20506), or L-alpha-glycerylphosphorylcholine (alpha-GPC) (Botany Bio, San Luis Obispo, Calif.; Product #alphagpc-99-powder) and 60% DMSO with 50 mM sodium acetate (pH 7) as follows:
1. 60% DMSO+50 mM Na-Acetate (pH 7)
2. 1.25M TMG+60% DMSO+50 mM Na-Acetate (pH 7)
3. 1M NDSB-195+60% DMSO+50 mM Na-Acetate (pH 7)
4. 1M NDSB-201+60% DMSO+50 mM Na-Acetate (pH 7)
5. 1M NDSB-221+60% DMSO+50 mM Na-Acetate (pH 7)
6. 1M Carnitine+60% DMSO+50 mM Na-Acetate (pH 7)
7. 750 mM Stachydrine+60% DMSO+50 mM Na-Acetate (pH 7)
8. 1M alpha-GPC+60% DMSO+50 mM Na-Acetate (pH 7)
9. −80° C.

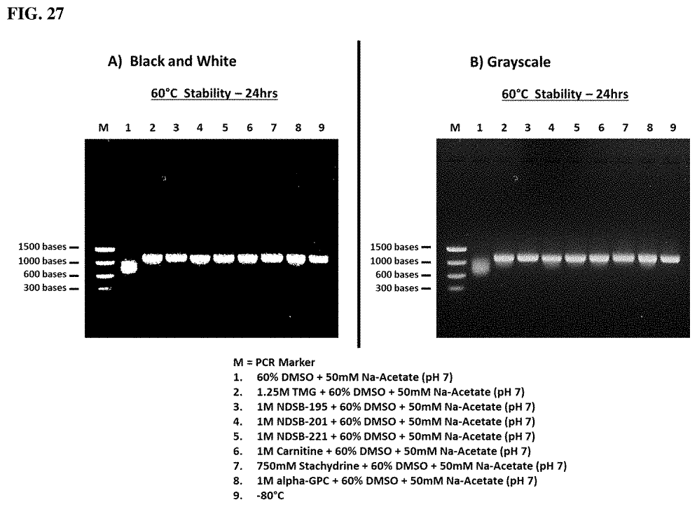

The stachydrine stock was adjusted to a pH of about 7 using NaOH. Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. FIG. 27 shows the agarose gel following storage of the RNA for about 24 hours at 60° C. Following about 24 hours at 60° C., samples 2-8 comprising either TMG, NDSB-195, NDSB-201, NDSB-221, carnitine, stachydrine, or alpha-GPC display significantly less RNA degradation as compared to sample 1 with only 60% DMSO and 50 mM sodium acetate (pH 7) as indicated by the bands of relatively comparable size and fluorescence intensity of samples 2-8 as compared to the −80° C. reference sample. Meanwhile, sample 1 with only 60% DMSO and 50 mM sodium acetate (pH 7) displays significant RNA degradation as indicated by the broadening of the RNA band, decreased fluorescence intensity, and lower apparent molecular weight.

### Example 28

Accelerated RNA Stability Testing at 70° C.

In vitro transcribed RNA was diluted at a ratio of about 1:10 (about 200-300 ug/mL) in different compositions containing either 50 mM Tris-HCl (pH 7), or a mixture of ˜8.5 kDa poly(2-(trimethylamino)ethyl methacrylate) chloride (PTMAEMA) (Sigma-Aldrich, St. Louis, Mo.; Product #657670) and 50 mM Tris-HCl (pH 7) as follows:
1. 50 mM Tris-HCl (pH 7)
2. 50 μM PTMAEMA+50 mM Tris-HCl (pH 7)
3. 40 μM PTMAEMA+50 mM Tris-HCl (pH 7)
4. 30 μM PTMAEMA+50 mM Tris-HCl (pH 7)
5. 20 μM PTMAEMA+50 mM Tris-HCl (pH 7)
6. −80° C.

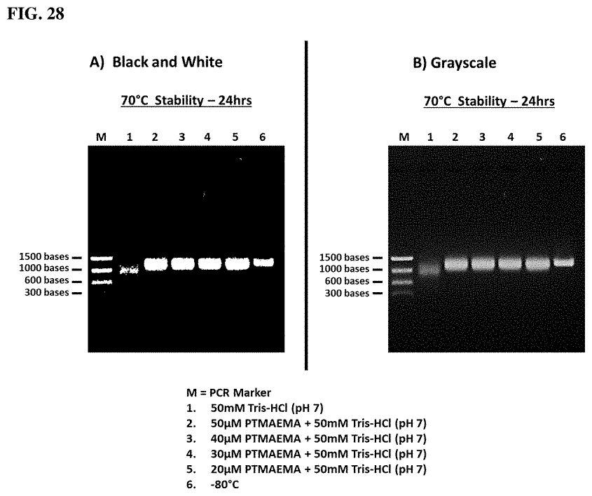

Following dilution of each sample in each respective RNA storage environment, samples were stored in a thermal cycler at 70° C. for about 24 hours. During storage at 70° C. samples were analyzed at selected timepoints by denaturing agarose gel electrophoresis to measure RNA degradation and the stability of the RNA samples in each RNA storage environment. Prior to performing gel electrophoresis, the PTMAEMA samples were incubated with 1 mM ˜8 kDa PAA sodium salt for 30 minutes at 70° C. and then incubated overnight at room temperature (about 20-25° C.) to improve analysis by gel electrophoresis. FIG. 28 shows the agarose gel following storage of the RNA for about 24 hours at 70° C. Following about 24 hours at 70° C., samples 2-5 comprising varying concentrations of ˜8.5 kDa PTMAEMA display significantly less RNA degradation as compared to sample 1 with only 50 mM Tris-HCl (pH 7) as indicated by the bands of relatively comparable size and fluorescence intensity of samples 2-5 as compared to the −80° C. reference sample. Meanwhile, sample 1 with only 50 mM Tris-HCl (pH 7) displays significant RNA degradation as indicated by the broadening of the RNA band, decreased fluorescence intensity, and lower apparent molecular weight.

### Example 29

Accelerated RNA Stability Testing at 70° C.

In vitro transcribed RNA was diluted at a ratio of about 1:10 (about 200-300 ug/mL) in different compositions containing either 50 mM Tris-HCl (pH 7), or a mixture of ˜8.5 kDa poly(diallyldimethylammonium chloride) (PDADMAC) (Polysciences Inc., Warrington, Pa.; Product #24828-100) and 50 mM Tris-HCl (pH 7) as follows:
1. 50 mM Tris-HCl (pH 7)
2. 100 μM PDADMAC+50 mM Tris-HCl (pH 7)
3. 90 μM PDADMAC+50 mM Tris-HCl (pH 7)
4. 80 μM PDADMAC+50 mM Tris-HCl (pH 7)
5. 70 μM PDADMAC+50 mM Tris-HCl (pH 7)
6. −80° C.

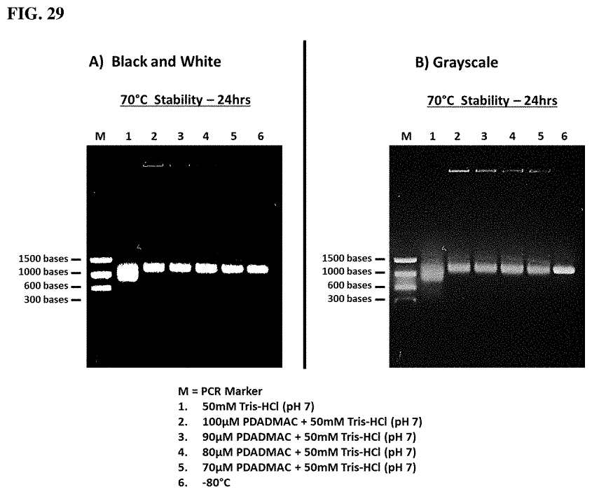

Following dilution of each sample in each respective RNA storage environment, samples were stored in a thermal cycler at 70° C. for about 24 hours. During storage at 70° C. samples were analyzed at selected timepoints by denaturing agarose gel electrophoresis to measure RNA degradation and the stability of the RNA samples in each RNA storage environment. Prior to performing gel electrophoresis, the PDADMAC samples were incubated with 1 mM ˜8 kDa PAA sodium salt overnight at room temperature (about 20-25° C.) to improve analysis by gel electrophoresis. FIG. 29 shows the agarose gel following storage of the RNA for about 24 hours at 70° C. Following about 24 hours at 70° C., samples 2-5 comprising varying concentrations of ˜8.5 kDa PDADMAC display significantly less RNA degradation as compared to sample 1 with only 50 mM Tris-HCl (pH 7) as indicated by the bands of relatively comparable size and fluorescence intensity of samples 2-5 as compared to the −80° C. reference sample. Meanwhile, sample 1 with only 50 mM Tris-HCl (pH 7) displays significant RNA degradation as indicated by the broadening of the RNA band, decreased fluorescence intensity, and lower apparent molecular weight.

### Example 30

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or a mixture of ˜10 kDa poly(2-(diethylamino)ethyl methacrylate) (PDEAEMA) (Sigma-Aldrich, St. Louis, Mo.; Product #910104) and 50 mM sodium acetate (pH 5.2) as follows:
1. 50 mM Na-Acetate (pH 5.2)
2. 35 μM PDEAEMA+50 mM Na-Acetate (pH 5.2)
3. 30 μM PDEAEMA+50 mM Na-Acetate (pH 5.2)
4. 25 μM PDEAEMA+50 mM Na-Acetate (pH 5.2)
5. 20 μM PDEAEMA+50 mM Na-Acetate (pH 5.2)
6. −80° C.

The PDEAEMA stock solution was made in 50 mM sodium acetate (pH 5.2). Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. Prior to performing gel electrophoresis, the PDEAEMA samples were incubated with 2.5 mM ˜8 kDa PAA sodium salt for about 1 hour at room temperature (about 20-25° C.) to improve analysis by gel electrophoresis. FIG. shows the agarose gel following storage of the RNA for about 24 hours at 60° C. Following about 24 hours at 60° C., samples 2-5 comprising varying concentrations of ˜10 kDa PDEAEMA display significantly less RNA degradation as compared to sample 1 with only 50 mM sodium acetate (pH 5.2) as indicated by the bands of relatively comparable size and fluorescence intensity of samples 2-5 as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 31

Accelerated RNA Stability Testing at 60° C.

In vitro transcribed RNA was diluted at a ratio of about 1:10 (about 200-300 ug/mL) in different compositions containing either 100 mM sodium benzoate with 5 mM sodium phosphate (pH 8) and 10 mM sodium acetate (pH 7), or a mixture of ˜7.5 kDa poly(2-(N-3-sulfopropyl-N,N-dimethyl ammonium)ethyl methacrylate) (PSBMA) (Sigma-Aldrich, St. Louis, Mo.; Product #922390) and 100 mM sodium benzoate with 5 mM sodium phosphate (pH 8) and 10 mM sodium acetate (pH 7) as follows:
1. 100 mM Na-Benzoate+5 mM Na-Phosphate (pH 8)+10 mM Na-Acetate (pH 7)
2. 5 mM PSBMA+100 mM Na-Benzoate+5 mM Na-Phosphate (pH 8)+10 mM Na-Acetate (pH 7)
3. 4 mM PSBMA+100 mM Na-Benzoate+5 mM Na-Phosphate (pH 8)+10 mM Na-Acetate (pH 7)
4. 3 mM PSBMA+100 mM Na-Benzoate+5 mM Na-Phosphate (pH 8)+10 mM Na-Acetate (pH 7)
5. 2 mM PSBMA+100 mM Na-Benzoate+5 mM Na-Phosphate (pH 8)+10 mM Na-Acetate (pH 7)
6. −80° C.

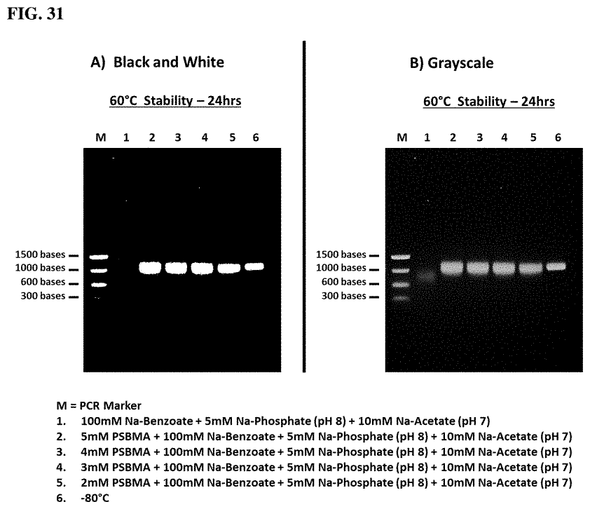

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. Prior to performing gel electrophoresis, the PSBMA samples were incubated with 1 mM ˜8 kDa PAA sodium salt for about 1 hour at room temperature (about 20-25° C.) to improve analysis by gel electrophoresis. FIG. 31 shows the agarose gel following storage of the RNA for about 24 hours at 60° C. Following about 24 hours at 60° C., samples 2-5 comprising varying concentrations of ˜7.5 kDa PSBMA display significantly less RNA degradation as compared to sample 1 with only 100 mM sodium benzoate with 5 mM sodium phosphate (pH 8) and 10 mM sodium acetate (pH 7) as indicated by the bands of relatively comparable size and fluorescence intensity of samples 2-5 as compared to the −80° C. reference sample. Meanwhile, sample 1 with only 100 mM sodium benzoate with 5 mM sodium phosphate (pH 8) and 10 mM sodium acetate (pH 7) displays significant RNA degradation as indicated by the broadening of the RNA band, decreased fluorescence intensity, and lower apparent molecular weight.

### Example 32

Accelerated RNA Stability Testing at 60° C.

In vitro transcribed RNA was diluted at a ratio of about 1:10 (about 200-300 ug/mL) in different compositions containing either 100 mM sodium benzoate with 5 mM sodium phosphate (pH 8) and 10 mM sodium acetate (pH 7), or a mixture of ˜9 kDa poly(2-methacryloyloxyethyl phosphorylcholine) (PMPC) (Sigma-Aldrich, St. Louis, Mo.; Product #922749) and 100 mM sodium benzoate with 5 mM sodium phosphate (pH 8) and 10 mM sodium acetate (pH 7) as follows:
1. 100 mM Na-Benzoate+5 mM Na-Phosphate (pH 8)+10 mM Na-Acetate (pH 7)
2. 5 mM PMPC+100 mM Na-Benzoate+5 mM Na-Phosphate (pH 8)+10 mM Na-Acetate (pH 7)
3. 4 mM PMPC+100 mM Na-Benzoate+5 mM Na-Phosphate (pH 8)+10 mM Na-Acetate (pH 7)
4. 3 mM PMPC+100 mM Na-Benzoate+5 mM Na-Phosphate (pH 8)+10 mM Na-Acetate (pH 7)
5. 2 mM PMPC+100 mM Na-Benzoate+5 mM Na-Phosphate (pH 8)+10 mM Na-Acetate (pH 7)
6. −80° C.

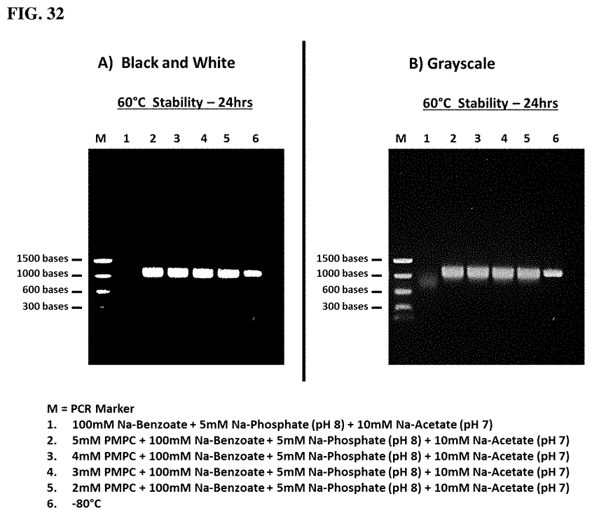

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. Prior to performing gel electrophoresis, the PMPC samples were incubated with 1 mM ˜8 kDa PAA sodium salt for about 1 hour at room temperature (about 20-25° C.) to improve analysis by gel electrophoresis. FIG. 32 shows the agarose gel following storage of the RNA for about 24 hours at 60° C. Following about 24 hours at 60° C., samples 2-5 comprising varying concentrations of ˜9 kDa PMPC display significantly less RNA degradation as compared to sample 1 with only 100 mM sodium benzoate with 5 mM sodium phosphate (pH 8) and 10 mM sodium acetate (pH 7) as indicated by the bands of relatively comparable size and fluorescence intensity of samples 2-5 as compared to the −80° C. reference sample. Meanwhile, sample 1 with only 100 mM sodium benzoate with 5 mM sodium phosphate (pH 8) and 10 mM sodium acetate (pH 7) displays significant RNA degradation as indicated by the broadening of the RNA band, decreased fluorescence intensity, and lower apparent molecular weight.

### Example 33

Accelerated RNA Stability Testing at 60° C.

In vitro transcribed RNA was diluted at a ratio of about 1:10 (about 200-300 ug/mL) in different compositions containing either 100 mM sodium benzoate with 5 mM sodium phosphate (pH 8) and 10 mM sodium acetate (pH 7), or a combination of both ˜7.5 kDa PSBMA and ˜9 kDa PMPC and 100 mM sodium benzoate with 5 mM sodium phosphate (pH 8) and 10 mM sodium acetate (pH 7) as follows:
1. 100 mM Na-Benzoate+5 mM Na-Phosphate (pH 8)+10 mM Na-Acetate (pH 7)
2. 2.5 mM PSBMA+2.5 mM PMPC+100 mM Na-Benzoate+5 mM Na-Phosphate (pH 8)+10 mM Na-Acetate (pH 7)
3. 2 mM PSBMA+2 mM PMPC+100 mM Na-Benzoate+5 mM Na-Phosphate (pH 8)+10 mM Na-Acetate (pH 7)
4. 1.5 mM PSBMA+1.5 mM PMPC+100 mM Na-Benzoate+5 mM Na-Phosphate (pH 8)+10 mM Na-Acetate (pH 7)
5. 1 mM PSBMA+1 mM PMPC+100 mM Na-Benzoate+5 mM Na-Phosphate (pH 8)+10 mM Na-Acetate (pH 7)
6. −80° C.

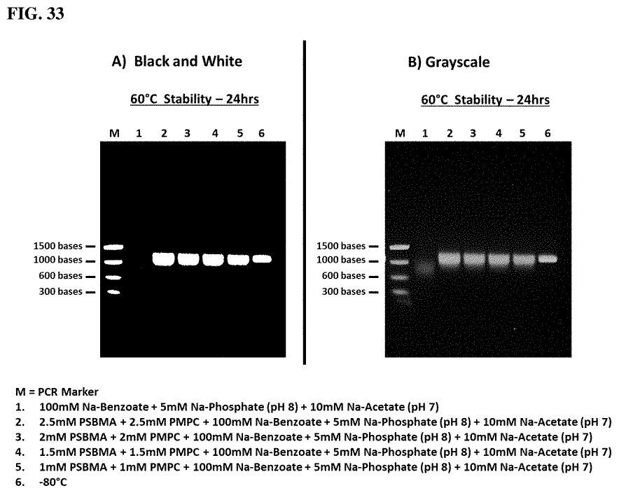

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. Prior to performing gel electrophoresis, the PSBMA+PMPC samples were incubated with 1 mM ˜8 kDa PAA sodium salt for about 1 hour at room temperature (about 20-25° C.) to improve analysis by gel electrophoresis. FIG. 33 shows the agarose gel following storage of the RNA for about 24 hours at 60° C. Following about 24 hours at 60° C., samples 2-5 comprising varying concentrations of combinations of both ˜7.5 kDa PSBMA and ˜9 kDa PMPC display significantly less RNA degradation as compared to sample 1 with only 100 mM sodium benzoate with 5 mM sodium phosphate (pH 8) and 10 mM sodium acetate (pH 7) as indicated by the bands of relatively comparable size and fluorescence intensity of samples 2-5 as compared to the −80° C. reference sample. Meanwhile, sample 1 with only 100 mM sodium benzoate with 5 mM sodium phosphate (pH 8) and 10 mM sodium acetate (pH 7) displays significant RNA degradation as indicated by the broadening of the RNA band, decreased fluorescence intensity, and lower apparent molecular weight.

### Example 34

Accelerated RNA Stability Testing at 60° C.

In vitro transcribed RNA was diluted at a ratio of about 1:10 (about 200-300 ug/mL) in different compositions containing either 100 mM sodium benzoate with 5 mM sodium phosphate (pH 8) and 10 mM sodium acetate (pH 7), or ˜7.5 kDa PSBMA, ˜9 kDa PMPC, a combination of both ˜7.5 kDa PSBMA and ˜9 kDa PMPC, PEG-block-PSBMA block copolymer (PEG-PSBMA) (PEG Mₙ 5,000; PSBMA Mₙ 13,000) (Sigma-Aldrich, St. Louis, Mo.; Product #925640), PEG-block-PMPC block copolymer (PEG-PMPC) (PEG Mₙ 5,000; PMPC Mₙ 21,000) (Sigma-Aldrich, St. Louis, Mo.; Product #925632), or ˜10 kDa polyvinylpyrrolidone (PVP) (Sigma-Aldrich, St. Louis, Mo.; Product #P2307) and 100 mM sodium benzoate with 5 mM sodium phosphate (pH 8) and 10 mM sodium acetate (pH 7) as follows:
1. 100 mM Na-Benzoate+5 mM Na-Phosphate (pH 8)+10 mM Na-Acetate (pH 7)
2. 5 mM PSBMA+100 mM Na-Benzoate+5 mM Na-Phosphate (pH 8)+10 mM Na-Acetate (pH 7)
3. 5 mM PMPC+100 mM Na-Benzoate+5 mM Na-Phosphate (pH 8)+10 mM Na-Acetate (pH 7)
4. 2.5 mM PSBMA+2.5 mM PMPC+100 mM Na-Benzoate+5 mM Na-Phosphate (pH 8)+10 mM Na-Acetate (pH 7)
5. 20 mg/mL PEG-PSBMA+100 mM Na-Benzoate+5 mM Na-Phosphate (pH 8)+10 mM Na-Acetate (pH 7)
6. 20 mg/mL PEG-PMPC+100 mM Na-Benzoate+5 mM Na-Phosphate (pH 8)+10 mM Na-Acetate (pH 7)
7. 2.5 mM PVP+100 mM Na-Benzoate+5 mM Na-Phosphate (pH 8)+10 mM Na-Acetate (pH 7)
8. −80° C.

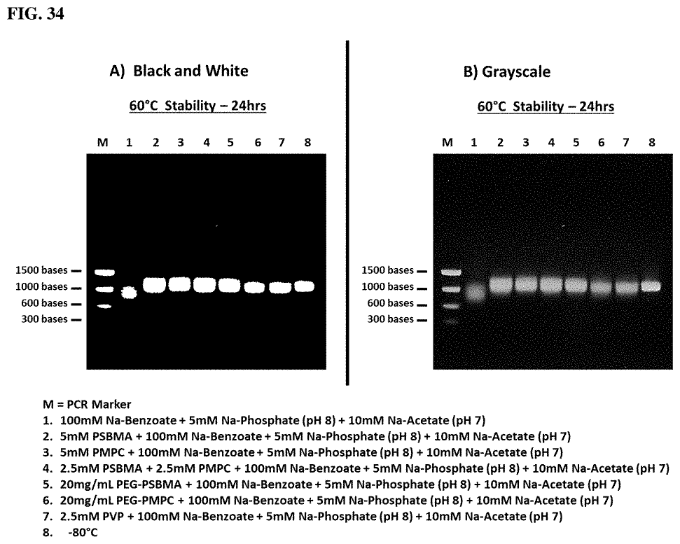

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. Prior to performing gel electrophoresis, the samples were incubated with 1 mM ˜8 kDa PAA sodium salt for about 1 hour at room temperature (about 20-25° C.) to improve analysis by gel electrophoresis. FIG. 34 shows the agarose gel following storage of the RNA for about 24 hours at 60° C. Following about 24 hours at 60° C., samples 2-7 comprising ˜7.5 kDa PSBMA, ˜9 kDa PMPC, a combination of both ˜7.5 kDa PSBMA and ˜9 kDa PMPC, PEG-PSBMA (PEG Mₙ 5,000; PSBMA Mₙ 13,000), PEG-PMPC (PEG Mₙ 5,000; PMPC Mₙ 21,000), or ˜10 kDa PVP display significantly less RNA degradation as compared to sample 1 with only 100 mM sodium benzoate with 5 mM sodium phosphate (pH 8) and 10 mM sodium acetate (pH 7) as indicated by the bands of relatively comparable size and fluorescence intensity of samples 2-7 as compared to the −80° C. reference sample. Meanwhile, sample 1 with only 100 mM sodium benzoate with 5 mM sodium phosphate (pH 8) and 10 mM sodium acetate (pH 7) displays significant RNA degradation as indicated by the broadening of the RNA band, decreased fluorescence intensity, and lower apparent molecular weight.

### Example 35

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or a mixture of ˜8 kDa poly(acrylic acid, sodium salt) (PAA) (Sigma-Aldrich, St. Louis, Mo.; Product #416029) and 50 mM sodium acetate (pH 5.2) as follows:
1. 50 mM Na-Acetate (pH 5.2)
2. 5 mM PAA sodium salt+50 mM Na-Acetate (pH 5.2)
3. 2.5 mM PAA sodium salt+50 mM Na-Acetate (pH 5.2)
4. 1 mM PAA sodium salt+50 mM Na-Acetate (pH 5.2)
5. 500 μM PAA sodium salt+50 mM Na-Acetate (pH 5.2)
6. −80° C.

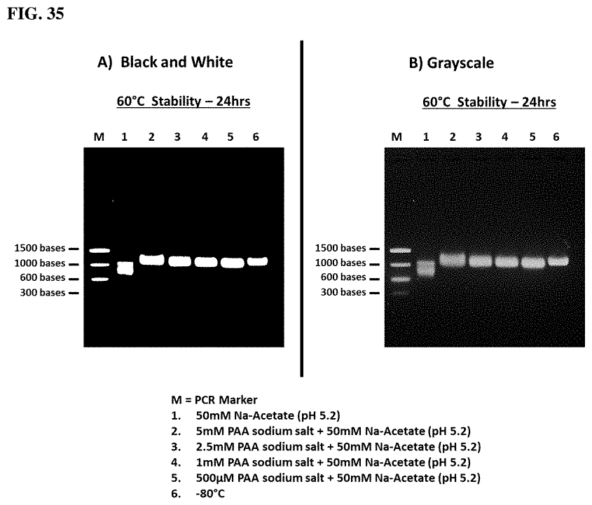

Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. FIG. 35 shows the agarose gel following storage of the RNA for about 24 hours at 60° C. Following about 24 hours at 60° C., samples 2-5 comprising varying concentrations of ˜8 kDa PAA display significantly less RNA degradation as compared to sample 1 with only 50 mM sodium acetate (pH 5.2) as indicated by the bands of relatively comparable size and fluorescence intensity of samples 2-5 as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 36

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or 50 μM-8.5 kDa PTMAEMA in combination with ˜7.5 kDa PSBMA, ˜9 kDa PMPC, a combination of both ˜7.5 kDa PSBMA and ˜9 kDa PMPC, PEG-PSBMA (PEG Mₙ 5,000; PSBMA Mₙ 13,000), PEG-PMPC (PEG Mₙ 5,000; PMPC Mₙ 21,000), poly(ethylene glycol) 8,000 (PEG) (Sigma-Aldrich, St. Louis, Mo.; Product #89510), poly(propylene glycol) 425 (PPG) (Mₙ ˜425) (Sigma-Aldrich, St. Louis, Mo.; Product #202304), or ˜10 kDa PVP, and 50 mM sodium acetate (pH 5.2) as follows:
1. 50 mM Na-Acetate (pH 5.2)
2. 100 μM PSBMA+50 μM PTMAEMA+50 mM Na-Acetate (pH 5.2)
3. 100 μM PMPC+50 μM PTMAEMA+50 mM Na-Acetate (pH 5.2)
4. 100 μM PSBMA+100 μM PMPC+50 μM PTMAEMA+50 mM Na-Acetate (pH 5.2)
5. 4 mg/mL PEG-PSBMA+50 μM PTMAEMA+50 mM Na-Acetate (pH 5.2)
6. 2 mg/mL PEG-PMPC+50 μM PTMAEMA+50 mM Na-Acetate (pH 5.2)
7. 1 mM PEG 8,000+50 μM PTMAEMA+50 mM Na-Acetate (pH 5.2)
8. 4 mg/mL PPG 425+50 μM PTMAEMA+50 mM Na-Acetate (pH 5.2)
9. 200 μM PVP+50 μM PTMAEMA+50 mM Na-Acetate (pH 5.2)
10. −80° C.

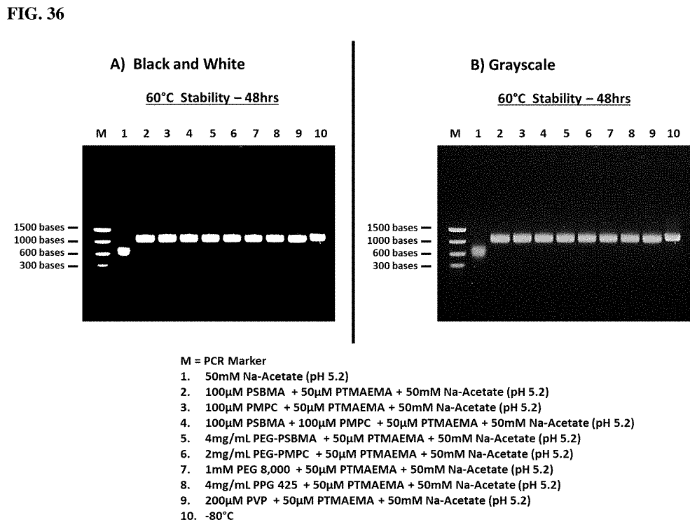

Following dilution of each sample in each respective RNA storage environment, samples were stored in a thermal cycler at 60° C. for about 48 hours. During storage at 60° C. samples were analyzed at selected timepoints by denaturing agarose gel electrophoresis to measure RNA degradation and the stability of the RNA samples in each RNA storage environment. Prior to performing gel electrophoresis, the samples were incubated with 2.5 mM ˜8 kDa PAA sodium salt for 30 minutes at 70° C. to improve analysis by gel electrophoresis. FIG. 36 shows the agarose gel following storage of the RNA for about 48 hours at 60° C. Following about 48 hours at 60° C., samples 2-9 comprising ˜8.5 kDa PTMAEMA and ˜7.5 kDa PSBMA, ˜9 kDa PMPC, a combination of both ˜7.5 kDa PSBMA and ˜9 kDa PMPC, PEG-PSBMA (PEG Mₙ 5,000; PSBMA Mₙ 13,000), PEG-PMPC (PEG Mₙ 5,000; PMPC Mₙ 21,000), PEG 8,000, PPG 425, or ˜10 kDa PVP display significantly less RNA degradation as compared to sample 1 with only 50 mM sodium acetate (pH 5.2) as indicated by the bands of relatively comparable size and fluorescence intensity of samples 2-9 as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 37

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or 100 μM-8.5 kDa PDADMAC in combination with ˜7.5 kDa PSBMA, ˜9 kDa PMPC, a combination of both ˜7.5 kDa PSBMA and ˜9 kDa PMPC, PEG-PSBMA (PEG Mₙ 5,000; PSBMA Mₙ 13,000), PEG-PMPC (PEG Mₙ 5,000; PMPC Mₙ 21,000), PEG 8,000, PPG 425, or ˜10 kDa PVP, and 50 mM sodium acetate (pH 5.2) as follows:
1. 50 mM Na-Acetate (pH 5.2)
2. 100 μM PSBMA+100 μM PDADMAC+50 mM Na-Acetate (pH 5.2)
3. 100 μM PMPC+100 μM PDADMAC+50 mM Na-Acetate (pH 5.2)
4. 100 μM PSBMA+100 μM PMPC+100 μM PDADMAC+50 mM Na-Acetate (pH 5.2)
5. 4 mg/mL PEG-PSBMA+100 μM PDADMAC+50 mM Na-Acetate (pH 5.2)
6. 2 mg/mL PEG-PMPC+100 μM PDADMAC+50 mM Na-Acetate (pH 5.2)
7. 1 mM PEG 8,000+100 μM PDADMAC+50 mM Na-Acetate (pH 5.2)
8. 4 mg/mL PPG 425+100 μM PDADMAC+50 mM Na-Acetate (pH 5.2)
9. 200 μM PVP+100 μM PDADMAC+50 mM Na-Acetate (pH 5.2)
10. −80° C.

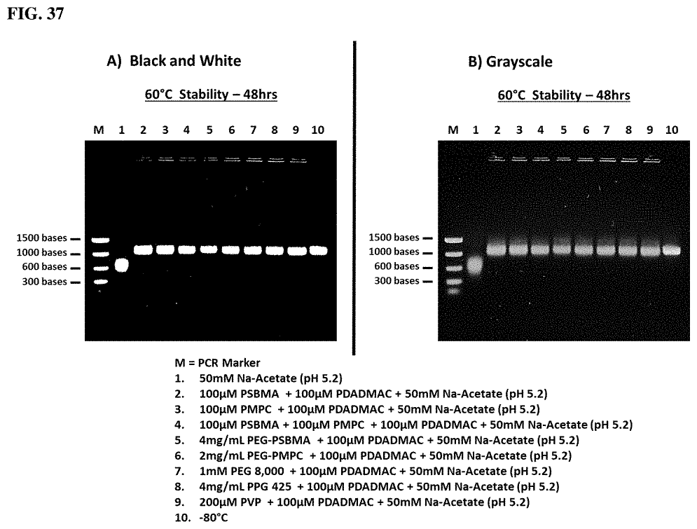

Following dilution of each sample in each respective RNA storage environment, samples were stored in a thermal cycler at 60° C. for about 48 hours. During storage at 60° C. samples were analyzed at selected timepoints by denaturing agarose gel electrophoresis to measure RNA degradation and the stability of the RNA samples in each RNA storage environment. Prior to performing gel electrophoresis, the samples were incubated with 2.5 mM ˜8 kDa PAA sodium salt for about 1 hour at room temperature (about 20-25° C.) to improve analysis by gel electrophoresis. FIG. 37 shows the agarose gel following storage of the RNA for about 48 hours at 60° C. Following about 48 hours at 60° C., samples 2-9 comprising ˜8.5 kDa PDADMAC and ˜7.5 kDa PSBMA, ˜9 kDa PMPC, a combination of both ˜7.5 kDa PSBMA and ˜9 kDa PMPC, PEG-PSBMA (PEG Mₙ 5,000; PSBMA Mₙ 13,000), PEG-PMPC (PEG Mₙ 5,000; PMPC Mₙ 21,000), PEG 8,000, PPG 425, or ˜10 kDa PVP display significantly less RNA degradation as compared to sample 1 with only 50 mM sodium acetate (pH 5.2) as indicated by the bands of relatively comparable size and fluorescence intensity of samples 2-9 as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 38

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or various combinations of more than one RNA stabilizing substance and 50 mM sodium acetate (pH 5.2) as follows:
1. 50 mM Na-Acetate (pH 5.2)
2. 50% DMSO+1M TMG+100 mM Na-Benzoate+50 mM Na-Acetate (pH 5.2)
3. 1M Ectoine+1M NDSB-195+1 mM PAA ˜8 kDa sodium salt+50 mM Na-Acetate (pH 5.2)
4. 100 mM Na-Quinolinic Acid (pH 7)+500 mM L-Proline+500 mM Choline-Cl+50 mM Na-Acetate (pH 5.2)
5. 100 mM DMSP+100 mM Na-Nicotinic Acid (pH 7)+400 mM NDSB-221+50% DMSO+50 mM Na-Acetate (pH 5.2)
6. 2 mM PMPC ˜9 kDa+400 mM Acetylcholine-Cl+25 mM Na-HMP+1M TMG+100 mM Na-Quinolinic Acid (pH 7)+400 mM Ectoine+50 mM Na-Acetate (pH 5.2)
7. −80° C.

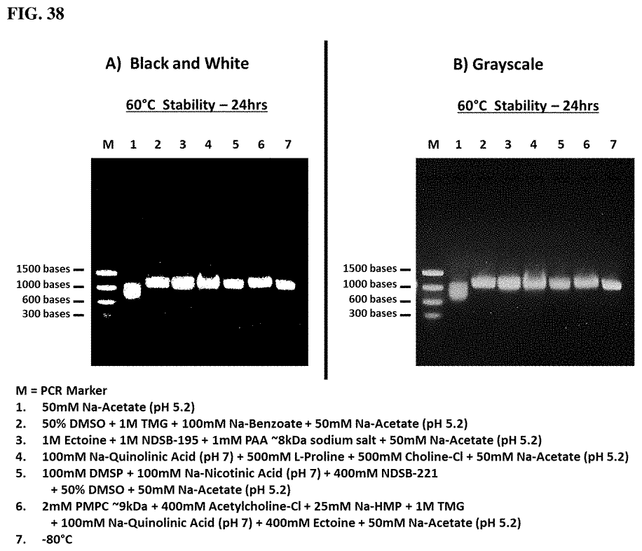

The quinolinic acid, nicotinic acid, and DMSP stocks were adjusted to a pH of about 7 using NaOH. Following dilution of each sample in each respective RNA storage environment, samples were stored in a thermal cycler at 60° C. for about 24 hours. During storage at 60° C. samples were analyzed at selected timepoints by denaturing agarose gel electrophoresis to measure RNA degradation and the stability of the RNA samples in each RNA storage environment. Prior to performing gel electrophoresis, sample 5 comprising DMSP was incubated with 1 mM ˜8 kDa PAA sodium salt for about 1 hour at room temperature (about 20-25° C.) to improve analysis by gel electrophoresis. FIG. 38 shows the agarose gel following storage of the RNA for about 24 hours at 60° C. Following about 24 hours at 60° C., samples 2-6 comprising various combinations of more than one RNA stabilizing substance display significantly less RNA degradation as compared to sample 1 with only 50 mM sodium acetate (pH 5.2) as indicated by the bands of relatively comparable size and fluorescence intensity of samples 2-6 as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 39

Accelerated RNA Stability Testing at 60° C.

In vitro transcribed RNA was diluted at a ratio of about 1:10 (about 200-300 ug/mL) in different compositions containing either 50 mM sodium acetate (pH 5.2), or various combinations of more than one RNA stabilizing substance and 50 mM sodium acetate (pH 5.2) as follows:
1. 50 mM Na-Acetate (pH 5.2)
2. 50% DMSO+1M TMG+100 mM Na-Benzoate+50 mM Na-Acetate (pH 5.2)
3. 100 mM Na-TMP+500 mM Choline-Cl+1 mM PAA ˜8 kDa sodium salt +50 mM Na-Acetate (pH 5.2)
4. 100 mM DMSP+100 mM Na-Quinolinic Acid (pH 7)+600 mM NDSB-195 +50% DMSO+50 mM Na-Acetate (pH 5.2)
5. 1 mM PSBMA ˜7.5 kDa+100 mM Acetylcholine-Cl+40 mM Na-HMP +1M TMG+100 mM Na-Benzoate+400 mM Ectoine+50 mM Na-Acetate (pH 5.2)
6. −80° C.

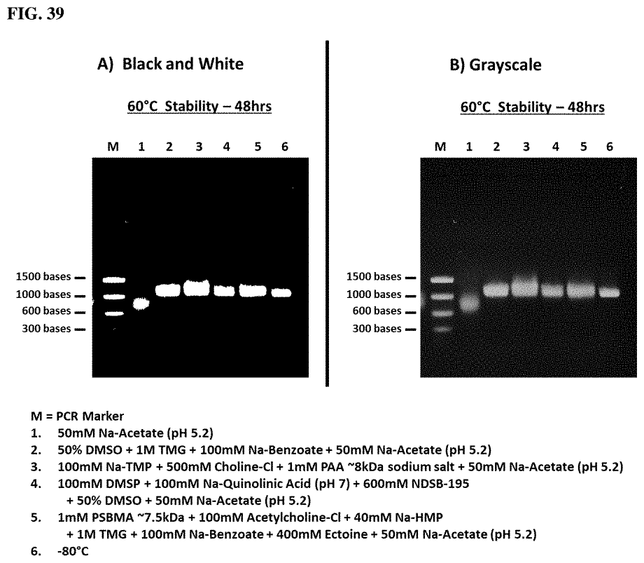

The quinolinic acid and DMSP stocks were adjusted to a pH of about 7 using NaOH. Following dilution of each sample in each respective RNA storage environment, samples were stored in a thermal cycler at 60° C. for about 48 hours. During storage at 60° C. samples were analyzed at selected timepoints by denaturing agarose gel electrophoresis to measure RNA degradation and the stability of the RNA samples in each RNA storage environment. Prior to performing gel electrophoresis, sample 4 comprising DMSP was incubated with 1 mM ˜8 kDa PAA sodium salt for about 1 hour at room temperature (about 20-25° C.) to improve analysis by gel electrophoresis. FIG. 39 shows the agarose gel following storage of the RNA for about 48 hours at 60° C. Following about 48 hours at 60° C., samples 2-5 comprising various combinations of more than one RNA stabilizing substance display significantly less RNA degradation as compared to sample 1 with only 50 mM sodium acetate (pH 5.2) as indicated by the bands of relatively comparable size and fluorescence intensity of samples 2-5 as compared to the −80° C. reference sample. Meanwhile, sample 1 with only 50 mM sodium acetate (pH 5.2) displays significant RNA degradation as indicated by the broadening of the RNA band, decreased fluorescence intensity, and lower apparent molecular weight.

### Example 40

Accelerated RNA Stability Testing at 60° C.

In vitro transcribed RNA was diluted at a ratio of about 1:10 (about 200-300 ug/mL) in different compositions containing either 50 mM Tris-HCl (pH 7), or various combinations of more than one RNA stabilizing substance and 50 mM Tris-HCl (pH 7) as follows:
1. 50 mM Tris-HCl (pH 7)
2. 25 mM Na-HMP+500 mM Choline-Cl+1M alpha-GPC+50 mM Tris-HCl (pH 7)
3. 200 mM Glycine+1 mM PSBMA ˜7.5 kDa+25 mM Na-HMP+1M L-Carnitine +200 mM Na-Benzoate+50 mM Tris-HCl (pH 7)
4. 50 μM PTMAEMA ˜8.5 kDa+1M TMG+400 mM Choline-Cl+400 mM L-Proline+2 mg/mL PEG-PSBMA (PEG Mn 5,000; PSBMA Mn 13,000)+50 mM Tris-HCl (pH 7)
5. −80° C.

Following dilution of each sample in each respective RNA storage environment, samples were stored in a thermal cycler at 60° C. for about 48 hours. During storage at 60° C. samples were analyzed at selected timepoints by denaturing agarose gel electrophoresis to measure RNA degradation and the stability of the RNA samples in each RNA storage environment. Prior to performing gel electrophoresis, sample 4 comprising PTMAEMA was incubated with 2.5 mM ˜8 kDa PAA sodium salt for 30 minutes at 70° C. to improve analysis by gel electrophoresis. FIG. shows the agarose gel following storage of the RNA for about 48 hours at 60° C. Following about 48 hours at 60° C., samples 2-4 comprising various combinations of more than one RNA stabilizing substance display significantly less RNA degradation as compared to sample 1 with only 50 mM Tris-HCl (pH 7) as indicated by the bands of relatively comparable size and fluorescence intensity of samples 2-4 as compared to the −80° C. reference sample. Meanwhile, sample 1 with only 50 mM Tris-HCl (pH 7) displays significant RNA degradation as indicated by the broadening of the RNA band, decreased fluorescence intensity, and lower apparent molecular weight.

### Example 41

Accelerated RNA Stability Testing at 60° C.

In vitro transcribed RNA was diluted at a ratio of about 1:10 (about 200-300 ug/mL) in different compositions containing either 50 mM Tris-HCl (pH 7), or various combinations of more than one RNA stabilizing substance and 50 mM Tris-HCl (pH 7) as follows:
1. 50 mM Tris-HCl (pH 7)
2. 40 mM Na-HMP+500 mM Choline-Cl+1M alpha-GPC+50 mM Tris-HCl (pH 7)
3. 500 mM Choline-Cl+50% DMSO+1 mM PMPC ˜9 kDa+500 mM NDSB-195 +50 mM Tris-HCl (pH 7)
4. 50 μM PTMAEMA ˜8.5 kDa+100 mM TMG+100 mM Ectoine+400 mM alpha-GPC+100 μM PSBMA ˜7.5 kDa+50 mM Tris-HCl (pH 7)
5. −80° C.

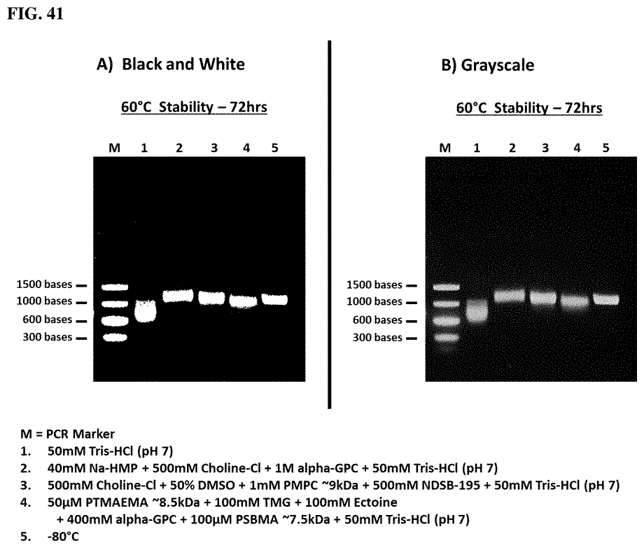

Following dilution of each sample in each respective RNA storage environment, samples were stored in a thermal cycler at 60° C. for about 72 hours. During storage at 60° C. samples were analyzed at selected timepoints by denaturing agarose gel electrophoresis to measure RNA degradation and the stability of the RNA samples in each RNA storage environment. Prior to performing gel electrophoresis, sample 4 comprising PTMAEMA was incubated with 2.5 mM ˜8 kDa PAA sodium salt for 30 minutes at 70° C. to improve analysis by gel electrophoresis. FIG. 41 shows the agarose gel following storage of the RNA for about 72 hours at 60° C. Following about 72 hours at 60° C., samples 2-4 comprising various combinations of more than one RNA stabilizing substance display significantly less RNA degradation as compared to sample 1 with only 50 mM Tris-HCl (pH 7) as indicated by the bands of relatively comparable size and fluorescence intensity of samples 2-4 as compared to the −80° C. reference sample. Meanwhile, sample 1 with only 50 mM Tris-HCl (pH 7) displays significant RNA degradation as indicated by the broadening of the RNA band, decreased fluorescence intensity, and lower apparent molecular weight.

1.-110. (canceled)

111.-292. (canceled)

293. A composition comprising at least one RNA substance and at least one RNA stabilizing substance, wherein said at least one RNA stabilizing substance comprises at least one of: a cyclic phosphate containing substance, a metaphosphate containing substance, a non-carbohydrate organic osmolyte substance, a quaternary ammonium containing substance, a quaternary phosphonium containing substance, a tertiary sulfonium containing substance, a hydrotrope containing substance, a betaine containing substance, a stabilizing monomer, and a stabilizing polymer.

294. The composition of claim 293, wherein said at least one RNA stabilizing substance is effective to maintain a defined level of stability of RNA molecules in said composition when subjected to a defined environment for a defined time period, wherein said defined environment includes temperatures in excess of −10° C., said time period is at least 72 hours, and said defined level of stability is defined by degradation of no more than about 50% of said RNA molecules.

295. The composition of claim 294, wherein said defined environment includes temperatures in excess of 0° C.

296. The composition of claim 294, wherein said defined environment includes temperatures in excess of 6° C.

297. The composition of claim 294, wherein said defined time period is at least 7 days.

298. The composition of claim 294, wherein said defined level of stability is defined by degradation of no more than 30% of said RNA molecules.

299. The composition of claim 293, further comprising at least one second RNA stabilizing substance comprising at least one of: an aprotic substance, a surfactant containing substance, glycerol, a polyether, polyethylene oxide, polyethylene glycol (PEG), polypropylene glycol (PPG), glycerol phosphate, diglycerol phosphate, methylpentane-2,4-diol, hexylene glycol, poly-HEMA, methylene phosphonic acid, methylene phosphonate, DTPMP, and ATMP.

300. The composition of claim 299, wherein said second RNA stabilizing substance is effective to maintain a defined level of stability of RNA molecules in said composition when subjected to a defined environment for a defined time period, wherein said defined environment includes temperatures in excess of −10° C., said time period is at least 72 hours, and said defined level of stability is defined by degradation of no more than about 50% of said RNA molecules.

301. The composition of claim 300, wherein said defined environment includes temperatures in excess of 0° C.

302. The composition of claim 300, wherein said defined environment includes temperatures in excess of 6° C.

303. The composition of claim 300, wherein said defined time period is at least 7 days.

304. The composition of claim 299, wherein said aprotic substance comprises at least one of: DMSO, diethyl sulfoxide, dimethyl sulfone, NMP, a carboxylate ester, a carbonate ester, a cyclic carbonate ester, a glycerin acetate, and a diester.

305. The composition of claim 293, wherein said cyclic phosphate containing substance or said metaphosphate containing substance comprises a substance with at least one of the formulas: [PO₃]ₘ, [HPO₃]ₘ, [NaPO₃]ₘ, and [KPO₃]ₘ, wherein m is an integer between about 2-100,000.

306. The composition of claim 305, wherein one or more counterions are substituted for one or more of H, Na, and K in said formulas.

307. The composition of claim 293, wherein said cyclic phosphate containing substance or said metaphosphate containing substance comprises at least one of: a metaphosphate, trimetaphosphate, tetrametaphosphate, hexametaphosphate, and cyclic-2,3-diphosphoglycerate.

308. The composition of claim 293, wherein said non-carbohydrate organic osmolyte substance comprises at least one of: an amino acid, an amino acid derivative, a beta-amino acid, a non-proteinogenic amino acid, a cyclic amino acid, and a low molecular weight aliphatic polyamine.

309. The composition of claim 308, wherein said low molecular weight aliphatic polyamine comprises at least one of: urea, cadaverine, putrescine, diaminopropane,1-3-diaminopropane, spermidine, spermine, thermospermine, norspermidine, norspermine, homocaldopentamine, hypusine, (aminopropyl)-cadaverine, N¹-(aminopropyl)-cadaverine, N¹,N′-bis(aminopropyl)-cadaverine, caldopentamine, caldohexamine, tris(3-aminopropyl)amine, N-acetylputrescine, N-acetylspermine, N¹-acetylspermidine, N¹-acetylspermine, and dimethylaminopropylamine.

310. The composition of claim 308, wherein said amino acid derivative comprises at least one of: ectoine, taurine, hydroxyectoine, N-acetylcysteine, N-acetylglutamate, aspartame, methionine sulfoxide, cystine, hydroxy proline, oxoproline, acetyl hydroxyproline, alanyl-glutamine, glutathione, carboxyglutamic acid, methyllysine, allysine, hydroxylysine, pyroglutamic acid, cystathionine, lanthionine, djenkolic acid, diaminopimelic acid, and pantothenic acid.

311. The composition of claim 308, wherein said non-proteinogenic amino acid comprises at least one of: pipecolic acid, gamma-aminobutyric acid, citrulline, aminolevulinic acid, homocysteine, aminobutyric acid, methylalanine, methylvaline, aminoisobutyric acid, norvaline, norleucine, and ornithine.

312. The composition of claim 293, wherein said non-carbohydrate organic osmolyte substance comprises at least one of: N-methylpipecolic acid, homoserine, statine, pyrrolysine, selenocysteine, gamma-carboxybutric acid, spermidic acid, aceglutamide, N-acetylaspartic acid, acetylcysteine, acetylleucine, sarcosine, N-methylglycine, N,N-dimethylglycine, dimethylglycine, N6-acetyl-lysine, CPHPC, dimethylalanine, glutaurine, 4-(gamma-glutamylamino)butanoic acid, beta-hydroxy beta-methylbutyric acid, beta-hydroxy beta-methylbutyrate, 3-hydroxyasparagine, lanthionine ketimine, N-methyl-L-glutamic acid, S-methylcysteine, 3-methylhistidine, N-methylornithine, milacemide, opine, nopaline, octopine, and agropine.

313. The composition of claim 293, wherein said tertiary sulfonium containing substance comprises at least one of: a hydroxy group, an ether bond, an ester bond, an amide bond, a carboxylate group, a carboxylic acid, an ester, a carboxylate ester, a carbonate ester, an amide group, an organosulfate group, a sulfonate group, and an organophosphate group.

314. The composition of claim 293, wherein said tertiary sulfonium containing substance comprises at least one of: a cation, an anion, and a zwitterion at about physiologic pH.

315. The composition of claim 293, wherein said tertiary sulfonium containing substance comprises at least one of: a ring structure and a heterocyclic ring structure.

316. The composition of claim 293, wherein said tertiary sulfonium containing substance comprises at least one polymer.

317. The composition of claim 293, wherein said tertiary sulfonium containing substance comprises at least one of: dimethylsulfoniopropionate, ethylmethylsulfoniopropionate, diethylsulfoniopropionate, isopropylmethylsulfoniopropionate, methylpropylsulfoniopropionate, tetramethylenesulfoniopropionate, dimethylsulfonioacetate, 2-dimethylsulfonioethanol, S-methylmethionine, and 4-dimethylsulfonio-2-hydroxybutyrate.

318. The composition of claim 293, wherein said quaternary ammonium containing substance comprises at least one of: a hydroxy group, an ether bond, an ester bond, an amide bond, a carboxylate group, a carboxylic acid, an ester, a carboxylate ester, a carbonate ester, an amide group, an organosulfate group, a sulfonate group, and an organophosphate group.

319. The composition of claim 293, wherein said quaternary ammonium containing substance comprises at least one of: a cation, an anion, and a zwitterion at about physiologic pH.

320. The composition of claim 293, wherein said quaternary ammonium containing substance comprises at least one of: a ring structure and a heterocyclic ring structure.

321. The composition of claim 293, wherein said quaternary ammonium containing substance comprises at least one of: an aliphatic quaternary ammonium and a quaternary ammonium nitrogen heterocycle.

322. The composition of claim 293, wherein said quaternary ammonium containing substance comprises at least one polymer.

323. The composition of claim 293, wherein said quaternary ammonium containing substance comprises at least one of: choline, cholamine, acetylcholine, methacholine, butyrylcholine, succinylmonocholine, a choline-based ester, bethanechol, carbachol, trimethyllysine, an imidazolium, a piperidinium, a pyridinium, a pyrrolidinium, tetramethonium, pentamethonium, tetraethylammonium, tetrapropylammonium, tetrabutylammonium, decamethonium, and hexamethonium.

324. The composition of claim 293, wherein said betaine containing substance comprises at least one of: a hydroxy group, an ether bond, an ester bond, an amide bond, a carboxylate group, a carboxylic acid, an ester, a carboxylate ester, a carbonate ester, an amide group, an organosulfate group, a sulfonate group, and an organophosphate group.

325. The composition of claim 293, wherein said betaine containing substance comprises at least one of: a ring structure and a heterocyclic ring structure.

326. The composition of claim 293, wherein said betaine containing substance comprises at least one of: a tertiary sulfonium, an aliphatic quaternary ammonium, a quaternary ammonium nitrogen heterocycle, and a non-detergent sulfobetaine.

327. The composition of claim 293, wherein said betaine containing substance comprises at least one polymer.

328. The composition of claim 293, wherein said betaine containing substance comprises at least one of: stachydrine, trimethylglycine, NDSB-195, NDSB-201, NDSB-211, NDSB-221, NDSB-223, NDSB-225, NDSB-256, NDSB-256-4T, carnitine, alpha-GPC, choline-O-sulfate, valine betaine, beta-alanine betaine, hydroxyproline betaine, gamma-butyrobetaine, trigonelline, isotrigonelline, homarine, a pyridinium, a pyrrolidinium, an imidazolium, a piperidinium, phenylalanine betaine, pipecolic acid betaine, betonicine, ergothioneine, hercynine, propionobetaine, pyridinium betaine, and phosphocholine.

329. The composition of claim 293, wherein said hydrotrope containing substance comprises at least one of: a phenyl, a phenol, an aryl, a pyridine, a pyridinium, a pyrrolidine, a pyrrolidinium, a pyrrole, an imidazole, an imidazolium, a pyrazole, a xanthine, a piperidine, a piperidinium, an indole, an indolium, and a methylxanthine.

330. The composition of claim 293, wherein said hydrotrope containing substance comprises at least one of: a polyphenol, a phenylalanine derivative, a tyrosine derivative, a tryptophan derivative, a phenylpropanoid, and a phenylpropanoid derivative.

331. The composition of claim 293, wherein said hydrotrope containing substance comprises at least one of: a hydroxy group, a carboxylate group, a carboxylic acid, an amide group, an organophosphate group, a phosphonate group, an organosulfate group, and a sulfonate group.

332. The composition of claim 293, wherein said hydrotrope containing substance comprises at least one of: a primary amine, a secondary amine, a tertiary amine, and a quaternary ammonium.

333. The composition of claim 293, wherein said hydrotrope containing substance comprises at least one of: benzoate, shikimate, caffeine, 1-methylnicotinamide, cafaminol, theacrine, thiamine, theobromine, gallic acid, a gallate, phenylalanine, tyrosine, a depside, an ellagitannin, a urolithin, a stilbenoid, a monolignol, a coumarin, a flavonoid, a cinnamic acid, a hydroxycinnamic acid, a cinnamic aldehyde, nicotinamide N-oxide, nicotinic acid N-oxide, dipicolinic acid, isonicotinamide, picolinic acid, urolithin A, casuarictin, luteic acid, gyrophoric acid, hexahydroxydiphenic acid, ellagic acid, nicotinic acid, quinolinic acid, trimesic acid, and coumaric acid.

334. The composition of claim 293, wherein said stabilizing monomer comprises at least one of: a cation, an anion, and a zwitterion at about physiologic pH.

335. The composition of claim 293, wherein said stabilizing monomer comprises at least one of: a carboxylic acid, a carboxylate group, a carboxylate ester, an organophosphate group, a phosphonic acid, a phosphonate, a sulfonic acid, a sulfonate group, and an organosulfate group.

336. The composition of claim 293, wherein said stabilizing monomer comprises at least one of: a quaternary ammonium, a tertiary sulfonium, a tertiary amine, and a quaternary phosphonium.

337. The composition of claim 293, wherein said stabilizing monomer comprises at least one of: a ring structure and a heterocyclic ring structure.

338. The composition of claim 293, wherein said stabilizing monomer comprises at least one of: an aliphatic quaternary ammonium, an aliphatic tertiary amine, a quaternary ammonium nitrogen heterocycle, a carboxybetaine, and a sulfobetaine.

339. The composition of claim 293, wherein said stabilizing monomer comprises at least one of: an aliphatic quaternary ammonium, an aliphatic tertiary amine, a nitrogen heterocycle, a pyrrolidine, a pyrrolidinium, a pyridine, a pyridinium, a piperidine, a piperidinium, an imidazole, and an imidazolium.

340. The composition of claim 293, wherein said stabilizing polymer comprises at least one of: a cation, an anion, and a zwitterion at about physiologic pH.

341. The composition of claim 293, wherein said stabilizing polymer comprises at least one of: a carboxylic acid, a carboxylate group, an ester, an ether, a carboxylate ester, a carbonate ester, an organophosphate group, a phosphonic acid, a phosphonate, a sulfonic acid, a sulfonate group, and an organosulfate group.

342. The composition of claim 293, wherein said stabilizing polymer comprises at least one of: a quaternary ammonium, a tertiary sulfonium, a tertiary amine, and a quaternary phosphonium.

343. The composition of claim 293, wherein said stabilizing polymer comprises at least two stabilizing monomers.

344. The composition of claim 343, wherein said at least two stabilizing monomers comprise at least one hydrolyzable bond.

345. The composition of claim 293, wherein said stabilizing polymer comprises at least one side group, wherein said side group comprises at least one of: a sidechain and a pendant group.

346. The composition of claim 345, wherein said at least one side group comprises at least one stabilizing monomer.

347. The composition of claim 346, wherein said at least one stabilizing monomer comprises at least one hydrolyzable bond.

348. The composition of claim 293, wherein said stabilizing polymer comprises at least one of: a ring structure and a heterocyclic ring structure.

349. The composition of claim 293, wherein said stabilizing polymer comprises at least one of: an aliphatic quaternary ammonium, an aliphatic tertiary amine, a quaternary ammonium nitrogen heterocycle, a betaine, a carboxybetaine, a phosphobetaine, and a sulfobetaine.

350. The composition of claim 293, wherein said stabilizing polymer comprises at least one of: an aliphatic quaternary ammonium, an aliphatic tertiary amine, a nitrogen heterocycle, a pyrrolidine, a pyrrolidinium, a pyridine, a pyridinium, a piperidine, a piperidinium, an imidazole, and an imidazolium.

351. The composition of claim 293, wherein said stabilizing polymer comprises at least one of: an aliphatic quaternary ammonium, a quaternary ammonium nitrogen heterocycle, a pyrrolidinium, a pyridinium, a piperidinium, and an imidazolium.

352. The composition of claim 293, wherein said stabilizing polymer comprises at least one of: an acrylate, a polyacrylate, an ester, a polyester, an ether, a polyether, an amide, a polyamide, a vinyl, a polyvinyl, oxazoline, a poly(oxazoline), acrylamide, a polyacrylamide, diallyldimethylammonium, polydiallyldimethylammonium, a cyclic amide, poly(vinylpyrrolidone), a phosphoester, a polyphosphoester, a phosphazene, a poly(phosphazene), an organophosphazene, a poly(organophosphazene), a siloxane, a polysiloxane, an imide, a polyimide, urea, polyurea, an amine, a polyamine, a quaternary ammonium amine, a quaternary ammonium polyamine, a tertiary amine, a tertiary amine polyamine, isocyanate, or polyurethane.

353. The composition of claim 293, wherein said stabilizing polymer comprises at least one of: an acrylate, polyacrylate, an ester, a polyester, an ether, a polyether, an amide, a polyamide, a vinyl, a polyvinyl, a poly(oxazoline), acrylamide, a polyacrylamide, diallyldimethylammonium, polydiallyldimethylammonium, poly(vinylpyrrolidone), a phosphazene, a poly(phosphazene), an organophosphazene, a poly(organophosphazene), a siloxane, a polysiloxane, an imide, a polyimide, urea, polyurea, isocyanate, and polyurethane.

354. The composition of claim 293, wherein said stabilizing polymer comprises at least one backbone chain comprising at least one of: an ester, a polyester, an ether, a polyether, an acrylate, a polyacrylate, polymethacrylate, polyacrylamide, polycarbonate, polylactic acid, polyglycolide, poly(lactic-co-glycolic-acid), polydioxanone, polyvinyl, poly(trimethylene carbonate), and polycaprolactone.

355. The composition of claim 293, wherein said stabilizing polymer comprises at least one of: poly(carboxybetaine acrylamide), poly(vinyl-pyridinio propanesulfonate), poly(vinylpyridine sulfobetaine), poly(vinylimidazole sulfobetaine), poly(vinylimidazole carboxybetaine), poly(2-methacryloxyethyldimethylsulfonium), poly(carboxybetaine methacrylate-ethyl ester), poly(2-(diethylamino)ethyl methacrylate), poly(sulfobetaine vinylpyridine), poly(sulfobetaine vinylimidazole), poly(carboxybetaine vinylimidazole), a poly(carboxybetaine methacrylate) ester, poly((carboxybetaine methacrylate)ethyl ester)), a poly(carboxybetaine methacrylate), a polyquaternium, hexadimethrine, poly(diallyldimethylammonium), a poly(carboxybetaine), a poly(sulfobetaine), a poly(phosphocholine), a poly(quaternary ammonium), a poly(dimethylsulfoniopropionate), poly(2-(trimethylamino)ethyl methacrylate), poly[(2-ethyldimethylammonioethyl methacrylate ethyl sulfate)-co-(1-vinylpyrrolidone)], poly(dimethlysulfonium ethylmethacrylate), poly(vinylpyrrolidone), poly(acrylic acid), PMPC, PSBMA, PEG-PSBMA, PEG-PMPC, Polyquat 11, poly(2-(dimethylamino)ethyl methacrylate), poly(2-(diisopropylamino)ethyl methacrylate), poly(vinyl-pyridinio propanesulfonate), and a poly(sulfobetaine methacrylate).

356. The composition of claim 293, wherein said stabilizing polymer comprises a copolymer.

357. The composition of claim 293, wherein said stabilizing polymer comprises at least one hydrolyzable bond.

358. The composition of claim 293, wherein said stabilizing polymer comprises at least one backbone chain comprising at least one hydrolyzable bond.

359. The composition of claim 293, wherein said stabilizing polymer comprises at least one a backbone repeat unit comprising at least one hydrolyzable bond.

360. The composition of claim 299, wherein said surfactant containing substance comprises at least one of: a cation, an anion, and a zwitterion at about physiologic pH.

361. The composition of claim 299, wherein said surfactant containing substance comprises at least one of: an organosulfate group, a sulfonate group, a carboxylate group, an imidazolium, a piperidinium, a pyridinium, a pyrrolidinium, and an aliphatic quaternary ammonium.

362. The composition of claim 293, wherein the total weight percentage of all said RNA stabilizing substances in said composition is at least 0.1 percent or at least one (1) nanomolar.

363. The composition of claim 299, wherein the total weight percentage of all said RNA stabilizing substances in said composition is at least 0.1 percent or at least one (1) nanomolar.

364. The composition of claim 293, further comprising at least one of: a cellular uptake agent, a chelating agent, a buffering agent, an inorganic salt, and a solvent.

365. The composition of claim 299, further comprising at least one of: a cellular uptake agent, a chelating agent, a buffering agent, an inorganic salt, and a solvent.

366. The composition of claim 293, wherein said at least one RNA substance comprises at least one coding RNA.

367. The composition of claim 366, wherein said at least one coding RNA comprises at least one of: mRNA and self-amplifying RNA.

368. The composition of claim 293, wherein said at least one RNA substance comprises at least one non-coding RNA.

369. The composition of claim 368, wherein said at least one non-coding RNA comprises at least one of: small interfering RNA (siRNA), microRNA, CRISPR RNA, antisense RNA (asRNA), small activating RNA, and RNA enzyme.

370. The composition of claim 293, wherein said composition is at least part of a pharmaceutical composition.

371. The composition of claim 370, wherein said pharmaceutical composition comprises at least one of: a medicament, a vaccine, a therapeutic agent, and a biostimulant.

372. The composition claim 293, wherein said composition is at least part of a bio stimulant composition.

373. The composition of claim 293, wherein said composition is at least part of an implant.

374. A method for use in producing an RNA product, comprising:
providing at least one RNA substance;
providing at least one RNA stabilizing substance, said RNA stabilizing substance being effective to stabilize RNA molecules for a defined use; and
combining said at least one RNA substance and said at least one RNA stabilizing substance.

375. The method of claim 374, wherein said RNA stabilizing substance is effective to maintain a defined level of stability of RNA molecules in said RNA product when subjected to a defined environment for a defined time period, wherein said defined environment includes temperatures in excess of −10° C., said time period is at least 72 hours, and said defined level of stability is defined by degradation of no more than about 50% of said RNA molecules.

376. The method of claim 375, wherein said defined environment includes temperatures in excess of 0° C.

377. The method of claim 375, wherein said defined environment includes temperatures in excess of 6° C.

378. The method of claim 375, wherein said defined time period is at least 7 days.

379. The method of claim 375, wherein said defined level of stability is defined by degradation of no more than 30% of said RNA molecules.

380. The method of claim 374, wherein said RNA product is stored in at least one of: a chamber and a container.

381. The method of claim 380, wherein said chamber comprises at least one of: a bottle, a container, an embedded complex, a vial, a tube, a jar, a syringe, a pre-filled syringe, a multi-chamber syringe, a prefilled multi-chamber syringe, a blister, a capsule, a tablet, a cartridge, an inhaler, a packet, a pod, a bag, and a box.

382. The method of claim 380, wherein said container is an embedded complex.

383. The method of claim 382, wherein said embedded complex is an implant.

384. The method of claim 374, wherein said defined use of said RNA product comprises use as an implant that is at least partly capable of delivering at least part of said at least one RNA substance to at least one tissue of at least one organism.

385. The method of claim 384, wherein said implant is at least partly capable of delivering said at least one RNA substance to at least one tissue of at least one animal.

386. The method of claim 384, wherein said implant is at least partly capable of delivering at least one RNA substance to at least one tissue of at least one human.

387. The method of claim 374, further comprising delivering said RNA product to at least one organism, wherein said at least one RNA substance is delivered to at least one tissue of said at least one organism.

388. The method of claim 387, wherein said at least one organism is an animal.

389. The method of claim 387, wherein said at least one organism is a mammal.

390. The method of claim 387, wherein said at least one organism is a human.

391. The method of claim 387, wherein said at least one organism is a plant.

392. The method of claim 374, wherein said defined use of said RNA product comprises use as at least one of: a medicament, a vaccine, a therapeutic agent, and a biostimulant.

393. The method of claim 374, wherein said defined use of said RNA product comprises use as a biostimulant.
